(19) United States
(12) Patent Application Publication
(10) Pub. No.: US 2021/0214459 A1
(43) Pub. Date: Jul. 15, 2021

(54) ANTIBODY MOLECULES TO CD73 AND USES THEREOF

(71) Applicants: Novartis AG, Basel (CH); SURFACE ONCOLOGY, INC., Cambridge, MA (US)

(72) Inventors: Ansgar Brock, San Diego, CA (US); Viviana Cremasco, Arlington, MA (US); Catherine Anne Sabatos-Peyton, Cambridge, MA (US); Glenn Dranoff, Sudbury, MA (US); Kulandayan Kasi Subramanian, Belmont, MA (US); Marie-Louise Fjaellskog, Lexington, MA (US); John Delmas Venable, III, San Diego, CA (US); Bianka Prinz, Lebanon, NH (US); Jerry M. Thomas, Lebanon, NH (US); Andrew Lake, Cambridge, MA (US); Scott Chappel, Cambridge, MA (US); Pamela Holland, Cambridge, MA (US); Michael Warren, Cambridge, MA (US); Alison Paterson, Cambridge, MA (US); Rachel W. O'Connor, Cambridge, MA (US); Michael Gladstone, Cambridge, MA (US); Jonathan Hill, Cambridge, MA (US); Christine Miller, Cambridge, MA (US)

(21) Appl. No.: 17/059,056

(22) PCT Filed: May 30, 2019
(86) PCT No.: PCT/US2019/034706

§ 371 Date: Nov. 25, 2020

Related U.S. Application Data
(60) Provisional application No. 62/688,119, filed on Jun. 21, 2018.
(60) Provisional application No. 62/678,708, filed on May 31, 2018.

(51) Int. Cl. C07K 16/28; G01N 33/573

(52) U.S. Cl. CPC C07K 16/2896; G01N 33/573; G01N 2333/916; A61K 2039/505; C07K 2317/92; C07K 2317/55; G01N 2800/52

(57) ABSTRACT

Antibody molecules that bind to CD73 are disclosed. The anti-CD73 antibody molecules can be used to treat, prevent and/or diagnose cancer.

### RELATED APPLICATIONS

This application claims priority to U.S. Ser. No. 62/678,708 filed on May 31, 2018, and U.S. Ser. No. 62/688,119 filed on Jun. 21, 2018, the contents of each of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on May 30, 2019, is named N2067-7154WO_SL.txt and is 480,336 bytes in size.

### BACKGROUND

Cluster of Differentiation 73 (CD73), also known as ecto-5′-nucleotidase (ecto-5′NT), is a glycosyl-phosphatidylinositol (GPI)-linked cell surface enzyme found in most tissues, and particularly expressed in endothelial cells and subsets of hematopoietic cells (Resta et al., Immunol Rev 161:95-109 (1998) and Colgan et al., Prinergic Signal 2:351-60 (2006)). CD73 catalyzes the conversion of adenosine monophosphate (AMP) to adenosine. Adenosine is a signaling molecule which mediates its biological effects through several receptors, including the Adenosine A1, A2A, A2B, and A3 receptors. The A2A receptor has received particular attention due to its broad expression on immune cells. Adenosine has pleiotropic effects in the tumor microenvironment, including expansion of regulatory T cells (Tregs), inhibition of effector T cell (Teff) responses mediated by interferon (IFN)-γ, and expansion of myeloid derived suppressor cells (MDSCs). See, e.g., Allard B, et al., Curr Opin Pharmacol 29:7-16 (2016) and Allard D, et al., Immunotherapy 8:145-163 (2016).

CD73 is also expressed on cancer cells, including colon, lung, pancreas, ovary, bladder, leukemia, glioma, glioblastoma, melanoma, thyroid, esophageal, prostate, and breast (Jin et al., Cancer Res 70:2245-55 (2010) and Stagg et al., PNAS 107: 1547-52 (2010); Zhang et al., Cancer Res 70:6407-11 (2010)). High CD73 expression has been reported to correlate with poor outcome across various cancer indications, such as lung, melanoma, triple-negative breast, squamous head and neck and colorectal cancers. See, e.g., Allard B, et al., Expert Opin Ther Targets 18:863-881 (2014); Leclerc B G, et al., Clin Cancer Res 22:158-166 (2016); Ren Z H, et al., Oncotarget 7:61690-61702 (2016); Ren Z H, et al., Oncol Lett 12:556-562 (2016); and Turcotte M, et al., Cancer Res 75:4494-4503 (2015).

Given the ongoing need for improved strategies for targeting diseases such as cancer, new compositions and methods for regulating CD73 activity and related therapeutic agents are highly desirable.

### SUMMARY

Disclosed herein are antibody molecules that bind to CD73 (Cluster of Differentiation 73) with high affinity and specificity. Nucleic acid molecules encoding the antibody molecules, expression vectors, host cells and methods for making the antibody molecules are also provided Immunoconjugates, multi- or bispecific antibody molecules and pharmaceutical compositions comprising the antibody molecules are also provided. The anti-CD73 antibody molecules disclosed herein can be used (alone or in combination with other agents or therapeutic modalities) to treat, prevent and/or diagnose disorders, including immune disorders and cancer. Thus, compositions and methods for treating and/or diagnosing various disorders, including cancer and immune disorders, using the anti-CD73 antibody molecules are disclosed herein.

Accordingly, in one aspect, this disclosure provides an antibody molecule (e.g., an isolated or recombinant antibody molecule) that binds to human CD73, comprising a heavy chain variable region (VH) comprising a heavy chain complementarity determining region 1 (VHCDR1) amino acid sequence of SEQ ID NO: 213, a VHCDR2 amino acid sequence of SEQ ID NO: 211; and/or a VHCDR3 amino acid sequence of 212, and/or a light chain variable region (VL) comprising a light chain complementarity determining region 1 (VLCDR1) amino acid sequence of SEQ ID NO: 221, a VLCDR2 amino acid sequence of SEQ ID NO: 222, and/or a VLCDR3 amino acid sequence of SEQ ID NO: 223. In certain embodiments, the antibody molecule comprises a VH comprising a VHCDR1 amino acid sequence of SEQ ID NO: 213, a VHCDR2 amino acid sequence of SEQ ID NO: 211, and a VHCDR3 amino acid sequence of SEQ ID NO: 212; and a VL comprising a VLCDR1 amino acid sequence of SEQ ID NO: 221, a VLCDR2 amino acid sequence of SEQ ID NO: 222, and a VLCDR3 amino acid sequence of SEQ ID NO: 223.

In one aspect, this disclosure provides an antibody molecule (e.g., an isolated or recombinant antibody molecule) that binds to human CD73, comprising a VH comprising a VHCDR1 amino acid sequence of SEQ ID NO: 210, a VHCDR2 amino acid sequence of SEQ ID NO: 211, and/or a VHCDR3 amino acid sequence of SEQ ID NO: 212; and/or a VL comprising a VLCDR1 amino acid sequence of SEQ ID NO: 221, a VLCDR2 amino acid sequence of SEQ ID NO: 222, and/or a VLCDR3 amino acid sequence of SEQ ID NO: 223. In some embodiments, the antibody molecule comprises a VH comprising a VHCDR1 amino acid sequence of SEQ ID NO: 210, a VHCDR2 amino acid sequence of SEQ ID NO: 211, and a VHCDR3 amino acid sequence of SEQ ID NO: 212; and a VL comprising a VLCDR1 amino acid sequence of SEQ ID NO: 221, a VLCDR2 amino acid sequence of SEQ ID NO: 222, and a VLCDR3 amino acid sequence of SEQ ID NO: 223.

In one aspect, this disclosure provides an antibody molecule (e.g., an isolated or recombinant antibody molecule) that binds to human CD73, comprising a VH comprising a VHCDR1 amino acid sequence of SEQ ID NO: 214, a VHCDR2 amino acid sequence of SEQ ID NO: 215, and/or a VHCDR3 amino acid sequence of SEQ ID NO: 212; and/or a VL comprising a VLCDR1 amino acid sequence of SEQ ID NO: 224, a VLCDR2 amino acid sequence of SEQ ID NO: 52, and/or a VLCDR3 amino acid sequence of SEQ ID NO: 225. In some embodiments, the antibody molecule comprises a VH comprising a VHCDR1 amino acid sequence of SEQ ID NO: 214, a VHCDR2 amino acid sequence of SEQ ID NO: 215, and a VHCDR3 amino acid sequence of SEQ ID NO: 212; and a VL comprising a VLCDR1 amino acid sequence of SEQ ID NO: 224, a VLCDR2 amino acid sequence of SEQ ID NO: 52, and a VLCDR3 amino acid sequence of SEQ ID NO: 225.

In one aspect, this disclosure provides an antibody molecule (e.g., an isolated or recombinant antibody molecule) that binds to human CD73, comprising a VH comprising a VHCDR1 amino acid sequence of SEQ ID NO: 216, a VHCDR2 amino acid sequence of SEQ ID NO: 217, and/or a VHCDR3 amino acid sequence of SEQ ID NO: 218; and/or a VL comprising a VLCDR1 amino acid sequence of SEQ ID NO: 226, a VLCDR2 amino acid sequence of SEQ ID NO: 52, and/or a VLCDR3 amino acid sequence of SEQ ID NO: 223. In some embodiments, the antibody molecule comprises a VH comprising a VHCDR1 amino acid sequence of SEQ ID NO: 216, a VHCDR2 amino acid sequence of SEQ ID NO: 217, and a VHCDR3 amino acid sequence of SEQ ID NO: 218; and a VL comprising a VLCDR1 amino acid sequence of SEQ ID NO: 226, a VLCDR2 amino acid sequence of SEQ ID NO: 52, and a VLCDR3 amino acid sequence of SEQ ID NO: 223.

In one aspect, this disclosure provides an antibody molecule (e.g., an isolated or recombinant antibody molecule) that binds to human CD73, comprising a VH comprising a VHCDR1 amino acid sequence of SEQ ID NO: 219, a VHCDR2 amino acid sequence of SEQ ID NO: 211, and/or a VHCDR3 amino acid sequence of SEQ ID NO: 218; and/or a VL comprising a VLCDR1 amino acid sequence of SEQ ID NO: 221, a VLCDR2 amino acid sequence of SEQ ID NO: 222, and/or a VLCDR3 amino acid sequence of SEQ ID NO: 223. In some embodiments, the antibody molecule comprises a VH comprising a VHCDR1 amino acid sequence of SEQ ID NO: 219, a VHCDR2 amino acid sequence of SEQ ID NO: 211, and a VHCDR3 amino acid sequence of SEQ ID NO: 218; and a VL comprising a VLCDR1 amino acid sequence of SEQ ID NO: 221, a VLCDR2 amino acid sequence of SEQ ID NO: 222, and a VLCDR3 amino acid sequence of SEQ ID NO: 223.

In certain embodiments, the antibody molecule comprises a heavy chain variable region comprising the amino acid sequence of SEQ ID NO: 220, or an amino acid sequence having at least about 85%, 90%, 95%, or 99% sequence identity to SEQ ID NO: 220. In certain embodiments, the antibody molecule comprises a light chain variable region comprising the amino acid sequence of SEQ ID NO: 227, or an amino acid sequence having at least about 85%, 90%, 95%, or 99% sequence identity to SEQ ID NO: 227. In certain embodiments, the antibody molecule comprises a heavy chain variable region comprising the amino acid sequence of SEQ ID NO: 220 (or a sequence having at least about 85%, 90%, 95%, or 99% sequence identity thereto) and a light chain variable region comprising the amino acid sequence of SEQ ID NO: 227 (or a sequence having at least about 85%, 90%, 95%, or 99% sequence identity thereto). In certain embodiments, the antibody molecule comprises a heavy chain variable region comprising the amino acid sequence of SEQ ID NO: 220 and a light chain variable region comprising the amino acid sequence of SEQ ID NO: 227. In certain embodiments, the antibody molecule comprises a heavy chain variable region having an amino acid sequence derived from a human VH4-34 germline sequence. In certain embodiments, the antibody molecule comprises a light chain variable region having an amino acid sequence derived from a human VK1-39 germline sequence. In certain embodiments, the antibody molecule comprises a heavy chain variable region having an amino acid sequence derived from a human VH4-34 germline sequence and a light chain variable region having an amino acid sequence derived from a human VK1-39 germline sequence.

In one aspect, this invention provides an antibody molecule that binds to human CD73, comprising a light chain variable region (VL) comprising a light chain complementarity determining region 1 (VLCDR1), a VLCDR2, and a VLCDR3 comprising the amino acid sequences of SEQ ID NOs: 344, 345, and 346, respectively. In one embodiment, the VLCDR1, VLCDR2, and VLCDR3 comprise the amino acid sequences of: (i) SEQ ID NOs: 221, 222, and 223, respectively; (ii) SEQ ID NOs: 221, 222, and 341, respectively; or (iii) SEQ ID NOs: 281, 15, and 282, respectively. In one embodiment, the antibody molecule comprises a heavy chain variable region (VH) comprising a heavy chain complementarity determining region 1 (VHCDR1), a VHCDR2, and a VHCDR3 comprising the amino acid sequences of: (i) SEQ ID NOs: 213, 211, and 212, respectively; (ii) SEQ ID NOs: 335, 288, and 334, respectively; or (iii) SEQ ID NOs: 273, 271, and 272, respectively. In one embodiment, the VHCDR1, VHCDR2, VHCDR3, VLCDR1, VLCDR2, and VLCDR3 comprise the amino acid sequences of: (i) SEQ ID NOs: 213, 211, 212, 221, 222, and 223, respectively; (ii) SEQ ID NOs: 335, 288, 334, 221, 222, and 341, respectively; or (iii) SEQ ID NOs: 273, 271, 272, 281, 15, and 282, respectively.

In one embodiment, the VHCDR1, VHCDR2, VHCDR3, VLCDR1, VLCDR2, and VLCDR3 comprise the amino acid sequences of SEQ ID NOs: 210, 211, 212, 221, 222, and 223, respectively. In one embodiment, the VHCDR1, VHCDR2, VHCDR3, VLCDR1, VLCDR2, and VLCDR3 comprise the amino acid sequences of SEQ ID NOs: 213, 211, 212, 221, 222, and 223, respectively. In one embodiment, the VHCDR1, VHCDR2, VHCDR3, VLCDR1, VLCDR2, and VLCDR3 comprise the amino acid sequences of SEQ ID NOs: 214, 215, 212, 224, 52, and 225, respectively. In one embodiment, the VHCDR1, VHCDR2, VHCDR3, VLCDR1, VLCDR2, and VLCDR3 comprise the amino acid sequences of SEQ ID NOs: 216, 217, 218, 226, 52, and 223, respectively. In one embodiment, the VHCDR1, VHCDR2, VHCDR3, VLCDR1, VLCDR2, and VLCDR3 comprise the amino acid sequences of SEQ ID NOs: 219, 211, 218, 221, 222, and 223, respectively.

In one embodiment, the VHCDR1, VHCDR2, VHCDR3, VLCDR1, VLCDR2, and VLCDR3 comprise the amino acid sequences of SEQ ID NOs: 333, 288, 334, 221, 222, and 341, respectively. In one embodiment, the VHCDR1, VHCDR2, VHCDR3, VLCDR1, VLCDR2, and VLCDR3 comprise the amino acid sequences of SEQ ID NOs: 335, 288, 334, 221, 222, and 341, respectively. In one embodiment, the VHCDR1, VHCDR2, VHCDR3, VLCDR1, VLCDR2, and VLCDR3 comprise the amino acid sequences of SEQ ID NOs: 336, 292, 334, 224, 52, and 342, respectively. In one embodiment, the VHCDR1, VHCDR2, VHCDR3, VLCDR1, VLCDR2, and VLCDR3 comprise the amino acid sequences of SEQ ID NOs: 337, 294, 338, 226, 52, and 341, respectively. In one embodiment, the VHCDR1, VHCDR2, VHCDR3, VLCDR1, VLCDR2, and VLCDR3 comprise the amino acid sequences of SEQ ID NOs: 339, 288, 338, 221, 222, and 341, respectively.

In one embodiment, the VHCDR1, VHCDR2, VHCDR3, VLCDR1, VLCDR2, and VLCDR3 comprise the amino acid sequences of SEQ ID NOs: 270, 271, 272, 281, 15, and 282, respectively. In one embodiment, the VHCDR1, VHCDR2, VHCDR3, VLCDR1, VLCDR2, and VLCDR3 comprise the amino acid sequences of SEQ ID NOs: 273, 271, 272, 281, 15, and 282, respectively. In one embodiment, the VHCDR1, VHCDR2, VHCDR3, VLCDR1, VLCDR2, and VLCDR3 comprise the amino acid sequences of SEQ ID NOs: 274, 275, 272, 283, 18, and 284, respectively. In one embodiment, the VHCDR1, VHCDR2, VHCDR3, VLCDR1, VLCDR2, and VLCDR3 comprise the amino acid sequences of SEQ ID NOs: 276, 277, 278, 285, 18, and 282, respectively. In one embodiment, the VHCDR1, VHCDR2, VHCDR3, VLCDR1, VLCDR2, and VLCDR3 comprise the amino acid sequences of SEQ ID NOs: 279, 271, 278, 281, 15, and 282, respectively.

In one embodiment, the antibody molecule comprises a VH comprising the amino acid sequence of SEQ ID NO: 220, 340, or 280, or an amino acid sequence having at least about 85%, 90%, 95%, or 99% sequence identity thereof. In one embodiment, the antibody molecule comprises a VL comprising the amino acid sequence of SEQ ID NO: 227, 343, or 286, or an amino acid sequence having at least about 85%, 90%, 95%, or 99% sequence identity thereof. In one embodiment, the antibody molecule comprises a VH and a VL comprising the amino acid sequences of: (i) SEQ ID NOs: 220 and 227, respectively; (ii) SEQ ID NOs: 340 and 343, respectively; or (iii) SEQ ID NOs: 280 and 286, respectively. In one embodiment, the antibody molecule comprises a VH and a VL comprising the amino acid sequences of SEQ ID NOs: 220 and 227, respectively. In one embodiment, the antibody molecule comprises a VH and a VL comprising the amino acid sequences of SEQ ID NOs: 340 and 343, respectively. In one embodiment, the antibody molecule comprises a VH and a VL comprising the amino acid sequences of SEQ ID NOs: 280 and 286, respectively. In one embodiment, the antibody molecule comprises a VH having an amino acid sequence derived from a human VH4-34, VH1-69, or VH4-39 germline sequence. In one embodiment, the antibody molecule comprises a VL having an amino acid sequence derived from a human VK1-39 germline sequence. In one embodiment, the antibody molecule comprises a VH having an amino acid sequence derived from a human VH4-34 germline sequence and a VL having an amino acid sequence derived from a human VK1-39 germline sequence. In one embodiment, the antibody molecule comprises a VH having an amino acid sequence derived from a human VH1-69 germline sequence and a VL having an amino acid sequence derived from a human VK1-39 germline sequence. In one embodiment, the antibody molecule comprises a VH having an amino acid sequence derived from a human VH4-39 germline sequence and a VL having an amino acid sequence derived from a human VK1-39 germline sequence.

In one aspect, provided herein is an antibody molecule that binds to human CD73, comprising a VH comprising a VHCDR1, a VHCDR2, and a VHCDR3, wherein: (i) the VHCDR1 comprises the amino acid sequence of SEQ ID NO: 347; (ii) the VHCDR2 comprises the amino acid sequence of SEQ ID NO: 288; and/or (iii) the VHCDR3 comprises the amino acid sequence of SEQ ID NO: 334 or 289. In one embodiment, the VHCDR1, VHCDR2, and VHCDR3 comprise the amino acid sequences of: (i) SEQ ID NOs: 335, 288, and 334, respectively; or (ii) SEQ ID NOs: 290, 288, and 289, respectively. In one embodiment, the antibody molecule comprises a VL comprising a VLCDR1, a VLCDR2, and a VLCDR3 comprising the amino acid sequences of: (i) SEQ ID NOs: 221, 222, and 341, respectively; or (ii) SEQ ID NOs: 298, 49, and 299, respectively. In one embodiment, the VHCDR1, VHCDR2, VHCDR3, VLCDR1, VLCDR2, and VLCDR3 comprise the amino acid sequences of: (i) SEQ ID NOs: 335, 288, 334, 221, 222, and 341, respectively; or (ii) SEQ ID NOs: 290, 288, 289, 298, 49, and 299, respectively.

In one embodiment, the VHCDR1, VHCDR2, VHCDR3, VLCDR1, VLCDR2, and VLCDR3 comprise the amino acid sequences of SEQ ID NOs: 287, 288, 289, 298, 49, and 299, respectively. In one embodiment, the VHCDR1, VHCDR2, VHCDR3, VLCDR1, VLCDR2, and VLCDR3 comprise the amino acid sequences of SEQ ID NOs: 290, 288, 289, 298, 49, and 299, respectively. In one embodiment, the VHCDR1, VHCDR2, VHCDR3, VLCDR1, VLCDR2, and VLCDR3 comprise the amino acid sequences of SEQ ID NOs: 291, 292, 289, 300, 52, and 301, respectively. In one embodiment, the VHCDR1, VHCDR2, VHCDR3, VLCDR1, VLCDR2, and VLCDR3 comprise the amino acid sequences of SEQ ID NOs: 293, 294, 295, 302, 52, and 299, respectively. In one embodiment, the VHCDR1, VHCDR2, VHCDR3, VLCDR1, VLCDR2, and VLCDR3 comprise the amino acid sequences of SEQ ID NOs: 296, 288, 295, 298, 49, and 299, respectively.

In one embodiment, the antibody molecule comprises a VH comprising the amino acid sequence of SEQ ID NO: 340 or 297, or an amino acid sequence having at least about 85%, 90%, 95%, or 99% sequence identity thereof. In one embodiment, the antibody molecule comprises a VL comprising the amino acid sequence of SEQ ID NO: 343 or 303, or an amino acid sequence having at least about 85%, 90%, 95%, or 99% sequence identity thereof. In one embodiment, the antibody molecule comprises a VH and a VL comprising the amino acid sequences of: (i) SEQ ID NOs: 340 and 343, respectively; or (ii) SEQ ID NOs: 297 and 303, respectively. In one embodiment, the antibody molecule comprises a VH and a VL comprising the amino acid sequences of SEQ ID NOs: 340 and 343, respectively. In one embodiment, the antibody molecule comprises a VH and a VL comprising the amino acid sequences of SEQ ID NOs: 297 and 303, respectively. In one embodiment, the antibody molecule comprises a VH having an amino acid sequence derived from a human VH1-69 germline sequence. In one embodiment, the antibody molecule comprises a VL having an amino acid sequence derived from a human VK1-39 or VK3-15 germline sequence. In one embodiment, the antibody molecule comprises a VH having an amino acid sequence derived from a human VH1-69 germline sequence and a VL having an amino acid sequence derived from a human VK1-39 germline sequence. In one embodiment, the antibody molecule comprises a VH having an amino acid sequence derived from a human VH1-69 germline sequence and a VL having an amino acid sequence derived from a human VK3-15 germline sequence.

In certain embodiments, the antibody molecule is a human antibody, a full length antibody, a bispecific antibody, Fab, F(ab′)2, Fv, or a single chain Fv fragment (scFv).

In certain embodiments, the antibody molecule comprises a heavy chain constant region selected from IgG1, IgG2, IgG3, and IgG4, and a light chain constant region chosen from the light chain constant regions of kappa or lambda. In certain embodiments, the antibody molecule comprises a heavy chain constant region comprising an amino acid sequence selected from the group consisting of SEQ ID NOs: 92-103, 119, and 120, and/or a light chain constant region comprising the amino acid sequence of SEQ ID NO: 104.

In one aspect, the disclosure provides an antibody molecule that competes for binding to human CD73 with an anti-CD73 antibody molecule disclosed herein, e.g., an anti-CD73 antibody molecule comprising a VH comprising the amino acid sequence of SEQ ID NO: 220 and a VL comprising the amino acid sequence of SEQ ID NO: 227. In one aspect, the disclosure provides an antibody molecule that binds to the same epitope as, substantially the same epitope as, an epitope that overlaps with, or an epitope that substantially overlaps with, the epitope of an anti-CD73 antibody molecule disclosed herein, e.g., an anti-CD73 antibody molecule comprising a VH comprising the amino acid sequence of SEQ ID NO: 220 and a VL comprising the amino acid sequence of SEQ ID NO: 227.

In one aspect, the disclosure provides a pharmaceutical composition comprising an anti-CD73 antibody molecule disclosed herein (e.g., an anti-CD73 antibody molecule comprising a VH comprising the amino acid sequence of SEQ ID NO: 220 and a VL comprising the amino acid sequence of SEQ ID NO: 227) and a pharmaceutically acceptable carrier, excipient or stabilizer.

In one aspect, the disclosure provides a nucleic acid encoding the antibody heavy or light chain variable region of an anti-CD73 antibody molecule disclosed herein (e.g., an anti-CD73 antibody molecule comprising a VH comprising the amino acid sequence of SEQ ID NO: 220 and a VL comprising the amino acid sequence of SEQ ID NO: 227). In one aspect, the disclosure provides an expression vector comprising a nucleic acid disclosed herein. In one aspect, the disclosure provides a host cell comprising a nucleic acid disclosed herein or an expression vector disclosed herein. In one aspect, the disclosure provides a method of producing an antibody molecule disclosed herein, the method comprising culturing a host cell disclosed herein under conditions suitable for gene expression.

In one aspect, the disclosure provides a method of detecting CD73 in a biological sample or in a subject, comprising (i) contacting the sample or the subject (and optionally, a reference sample or subject) with an anti-CD73 antibody molecule disclosed herein (e.g., an anti-CD73 antibody molecule comprising a VH comprising the amino acid sequence of SEQ ID NO: 220 and a VL comprising the amino acid sequence of SEQ ID NO: 227) under conditions that allow interaction of the antibody molecule and CD73 to occur, and (ii) detecting formation of a complex between the antibody molecule and CD73.

In one aspect, disclosed herein is a method of detecting soluble CD73 in a biological sample (e.g., serum or plasma) from a subject, comprising (i) contacting the biological sample (e.g., serum or plasma) with an anti-CD73 antibody molecule disclosed herein (e.g., an anti-CD73 antibody molecule comprising a VH comprising the amino acid sequence of SEQ ID NO: 220 and a VL comprising the amino acid sequence of SEQ ID NO: 227) under conditions that allow interaction of the antibody molecule and soluble CD73 to occur, and (ii) detecting formation of a complex between the antibody molecule and soluble CD73. In some embodiments, the subject has received or is going to receive administration of a therapeutic agent, e.g., one, two or all of a therapeutic anti-CD73 antibody molecule, a PD-1 inhibitor and an adenosine A2AR antagonist.

In one aspect, disclosed herein is a method of evaluating responsive of a subject to a therapeutic agent, wherein the subject has received or is going to receive administration of the therapeutic agent (e.g., one, two or all of a therapeutic anti-CD73 antibody molecule, a PD-1 inhibitor and an adenosine A2AR antagonist), comprising acquiring the level of soluble CD73 in the subject, e.g., at one or more time points prior to, during, or after the administration of the therapeutic agent, wherein acquiring the level of soluble CD73 comprises, e.g., contacting the subject, e.g., a biological sample (e.g., serum or plasma) from the subject, with an anti-CD73 antibody molecule disclosed herein (e.g., an anti-CD73 antibody molecule comprising a VH comprising the amino acid sequence of SEQ ID NO: 220 and a VL comprising the amino acid sequence of SEQ ID NO: 227) under conditions that allow interaction of the antibody molecule and soluble CD73 to occur, and detecting formation of a complex between the antibody molecule and soluble CD73, wherein the level of soluble CD73 is indicative of the responsiveness of the subject to the therapeutic agent.

In one aspect, disclosed herein is a method of evaluating effectiveness of a therapeutic agent in a subject, wherein the subject has received or is going to receive administration of the therapeutic agent (e.g., one, two or all of a therapeutic anti-CD73 antibody molecule, a PD-1 inhibitor and an adenosine A2AR antagonist), comprising acquiring the level of soluble CD73 in the subject, e.g., at one or more time points prior to, during, or after the administration of the therapeutic agent, wherein acquiring the level of soluble CD73 comprises, e.g., contacting the subject, e.g., a biological sample (e.g., serum or plasma) from the subject, with an anti-CD73 antibody molecule disclosed herein (e.g., an anti-CD73 antibody molecule comprising a VH comprising the amino acid sequence of SEQ ID NO: 220 and a VL comprising the amino acid sequence of SEQ ID NO: 227) under conditions that allow interaction of the antibody molecule and soluble CD73 to occur, and detecting formation of a complex between the antibody molecule and soluble CD73, wherein the level of soluble CD73 is indicative of the effectiveness of the therapeutic agent in the subject.

In some embodiments of the aforementioned aspects or embodiments, the therapeutic anti-CD73 antibody molecule is an anti-CD73 antibody molecule disclosed herein. In some embodiments, the therapeutic anti-CD73 antibody molecule comprises: (i) a VH comprising a VHCDR1 amino acid sequence of SEQ ID NO: 38, a VHCDR2 amino acid sequence of SEQ ID NO: 36, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; (ii) a VH comprising a VHCDR1 amino acid sequence of SEQ ID NO: 72, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; (iii) a VH comprising a VHCDR1 amino acid sequence of SEQ ID NO: 38, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; (iv) a VH comprising a VHCDR1 amino acid sequence of SEQ ID NO: 137, a VHCDR2 amino acid sequence of SEQ ID NO: 136, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; (v) a VH comprising a VHCDR1 amino acid sequence of SEQ ID NO: 137, a VHCDR2 amino acid sequence of SEQ ID NO: 146, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; (vi) a VH comprising a VHCDR1 amino acid sequence of SEQ ID NO: 137, a VHCDR2 amino acid sequence of SEQ ID NO: 154, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; (vii) a VH comprising a VHCDR1 amino acid sequence of SEQ ID NO: 61, a VHCDR2 amino acid sequence of SEQ ID NO: 60, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16; (viii) a VH comprising a VHCDR1 amino acid sequence of SEQ ID NO: 4, a VHCDR2 amino acid sequence of SEQ ID NO: 26, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16; (ix) a VH comprising a VHCDR1 amino acid sequence of SEQ ID NO: 4, a VHCDR2 amino acid sequence of SEQ ID NO: 2, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16, or (x) a VH comprising a VHCDR1 amino acid sequence of SEQ ID NO: 163, a VHCDR2 amino acid sequence of SEQ ID NO: 162, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16. In some embodiments, the therapeutic anti-CD73 antibody molecule comprises: (i) a heavy chain variable region comprising the amino acid sequence of SEQ ID NO: 44 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto); (ii) a heavy chain variable region comprising the amino acid sequence of SEQ ID NO: 77 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto); (iii) a heavy chain variable region comprising the amino acid sequence of SEQ ID NO: 84 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto); (iv) a heavy chain variable region comprising the amino acid sequence of SEQ ID NO: 142 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto); (v) a heavy chain variable region comprising the amino acid sequence of SEQ ID NO: 151 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto); (vi) a heavy chain variable region comprising the amino acid sequence of SEQ ID NO: 159 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto); (vii) a heavy chain variable region comprising the amino acid sequence of SEQ ID NO: 66 (or a sequence having at least about 85%, 90%, 95%, or 99% sequence identity thereto) and a light chain variable region comprising the amino acid sequence of SEQ ID NO: 21 (or a sequence having at least about 85%, 90%, 95%, or 99% sequence identity thereto); (viii) a heavy chain variable region comprising the amino acid sequence of SEQ ID NO: 31 (or a sequence having at least about 85%, 90%, 95%, or 99% sequence identity thereto) and a light chain variable region comprising the amino acid sequence of SEQ ID NO: 21 (or a sequence having at least about 85%, 90%, 95%, or 99% sequence identity thereto); (ix) a heavy chain variable region comprising the amino acid sequence of SEQ ID NO: 10 (or a sequence having at least about 85%, 90%, 95%, or 99% sequence identity thereto) and a light chain variable region comprising the amino acid sequence of SEQ ID NO: 21 (or a sequence having at least about 85%, 90%, 95%, or 99% sequence identity thereto); or (x) a heavy chain variable region comprising the amino acid sequence of SEQ ID NO: 168 (or a sequence having at least about 85%, 90%, 95%, or 99% sequence identity thereto) and a light chain variable region comprising the amino acid sequence of SEQ ID NO: 21 (or a sequence having at least about 85%, 90%, 95%, or 99% sequence identity thereto). In some embodiments, the therapeutic anti-CD73 antibody molecule comprises: (i) a heavy chain comprising the amino acid sequence of SEQ ID NO: 46 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto); (ii) a heavy chain comprising the amino acid sequence of SEQ ID NO: 114 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto); (iii) a heavy chain comprising the amino acid sequence of SEQ ID NO: 79 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto); (iv) a heavy chain comprising the amino acid sequence of SEQ ID NO: 116 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto); (v) a heavy chain comprising the amino acid sequence of SEQ ID NO: 86 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto); (vi) a heavy chain comprising the amino acid sequence of SEQ ID NO: 117 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto); (vii) a heavy chain comprising the amino acid sequence of SEQ ID NO: 68 (or a sequence having at least about 85%, 90%, 95%, or 99% sequence identity thereto) and a light chain comprising the amino acid sequence of SEQ ID NO: 23 (or a sequence having at least about 85%, 90%, 95%, or 99% sequence identity thereto); (viii) a heavy chain comprising the amino acid sequence of SEQ ID NO: 115 (or a sequence having at least about 85%, 90%, 95%, or 99% sequence identity thereto) and a light chain comprising the amino acid sequence of SEQ ID NO: 23 (or a sequence having at least about 85%, 90%, 95%, or 99% sequence identity thereto); (ix) a heavy chain comprising the amino acid sequence of SEQ ID NO: 33 (or a sequence having at least about 85%, 90%, 95%, or 99% sequence identity thereto) and a light chain comprising the amino acid sequence of SEQ ID NO: 23 (or a sequence having at least about 85%, 90%, 95%, or 99% sequence identity thereto); (x) a heavy chain comprising the amino acid sequence of SEQ ID NO: 113 (or a sequence having at least about 85%, 90%, 95%, or 99% sequence identity thereto) and a light chain comprising the amino acid sequence of SEQ ID NO: 23 (or a sequence having at least about 85%, 90%, 95%, or 99% sequence identity thereto); (xi) a heavy chain comprising the amino acid sequence of SEQ ID NO: 12 (or a sequence having at least about 85%, 90%, 95%, or 99% sequence identity thereto) and a light chain comprising the amino acid sequence of SEQ ID NO: 23 (or a sequence having at least about 85%, 90%, 95%, or 99% sequence identity thereto); or (xii) a heavy chain comprising the amino acid sequence of SEQ ID NO: 112 (or a sequence having at least about 85%, 90%, 95%, or 99% sequence identity thereto) and a light chain comprising the amino acid sequence of SEQ ID NO: 23 (or a sequence having at least about 85%, 90%, 95%, or 99% sequence identity thereto).

In some embodiments of the aforementioned aspects or embodiments, the PD-1 inhibitor is a PD-1 inhibitor described herein.

In some embodiments of the aforementioned aspects or embodiments, the adenosine A2AR antagonist is an adenosine A2AR antagonist described herein.

In one aspect, disclosed herein is a system for detecting soluble CD73 in a biological sample (e.g., serum or plasma) from a subject, comprising:

(i) an anti-CD73 antibody molecule described herein, e g, immobilized on a solid support, for contacting the biological sample to form a complex between the antibody molecule and soluble CD73 in the biological sample, and

(ii) a detecting reagent, e.g., a labeled anti-CD73 antibody molecule, for contacting the complex thereby detecting the soluble CD73 in the biological sample.

In some embodiments of the aforementioned aspects or embodiments, in addition to administration of said therapeutic agent, (e.g., one, two or all of a therapeutic anti-CD73 antibody molecule, a PD-1 inhibitor and an adenosine A2AR antagonist), one or more of the following are administered: (i) a CTLA-4 inhibitor, optionally wherein the CTLA-4 inhibitor is Ipilimumab or Tremelimumab; (ii) a TIM-3 inhibitor, optionally wherein the TIM-3 inhibitor is selected from the group consisting of MGB453, TSR-022, and LY3321367; (iii) a LAG-3 inhibitor, optionally wherein the LAG-3 inhibitor is selected from the group consisting of LAG525, BMS-986016, TSR-033, MK-4280 and REGN3767; (iv) a GITR agonist, optionally wherein the GITR agonist is selected from the group consisting of GWN323, BMS-986156, MK-4166, MK-1248, TRX518, INCAGN1876, AMG 228, and INBRX-110; (v) an anti-CD3 multispecific antibody molecule, optionally wherein the anti-CD3 multispecific antibody molecule is an anti-CD3×anti-CD123 bispecific antibody molecule (e.g., XENP14045), or an anti-CD3×anti-CD20 bispecific antibody molecule (e.g., XENP13676); (vi) a cytokine molecule, optionally wherein the cytokine molecule is IL-15 complexed with a soluble form of IL-15 receptor alpha (IL-15Ra); (vii) a STING agonist; (viii) a macrophage colony-stimulating factor (M-CSF) inhibitor, optionally wherein the M-CSF inhibitor is MCS110; (ix) a CSF-1R inhibitor, optionally wherein the CSF-1R inhibitor is BLZ945; (x) an inhibitor of indoleamine 2,3-dioxygenase (IDO) and/or tryptophan 2,3-dioxygenase (TDO); (xi) a TGF-β inhibitor; (xii) an oncolytic vaccine; (xiii) a chimeric antigen receptor (CAR) T-cell therapy, optionally wherein the CAR T-cell therapy is CTL019; or (xiv) a compound disclosed in Table 18.

In some embodiments of the aforementioned aspects or embodiments, the therapeutic anti-CD73 antibody molecule is administered at a dose of about 100 mg to 1600 mg, about 100 mg to 1400 mg, about 100 mg to 1200 mg, about 100 mg to 1000 mg, about 100 mg to 800 mg, about 100 mg to 600 mg, about 100 mg to 400 mg, about 100 mg to 200 mg, or about 100 mg, about 180 mg, or about 200 mg, once every two weeks (Q2W), optionally wherein the antibody molecule is administered at a dose of at least about 180 mg Q2W. In some embodiments, the therapeutic anti-CD73 antibody molecule is administered, e.g., intravenously, at a dose of about 5 mg to 100 mg, about 100 mg to 500 mg, about 500 mg to 1000 mg, about 1000 mg to 1500 mg, about 1500 mg to 2000 mg, about 2000 mg to 2500 mg, about 2500 mg to 3000 mg, about 3000 mg to 3500 mg, or about 3500 mg to 4000 mg, e.g., at a dose of about 6 mg, about 20 mg, about 60 mg, about 200 mg, about 600 mg, about 1200 mg, about 2400 mg, about 3000 mg, or about 3600 mg, e.g., once every week (QW), once every two weeks (Q2W), or once every four weeks (Q4W), e.g., Q2W.

In some embodiments, the therapeutic anti-CD73 antibody molecule is administered in combination with a PD-1 inhibitor, optionally wherein the PD-1 inhibitor is selected from the group consisting of PDR001, Nivolumab, Pembrolizumab, Pidilizumab, MEDI0680, REGN2810, TSR-042, PF-06801591, and AMP-224, optionally wherein: the PD-1 inhibitor is an anti-PD-1 antibody molecule, wherein the anti-PD-1 antibody molecule is administered, e.g., intravenously, at a dose of about 250 mg to 350 mg, about 350 mg to 450 mg, or about 450 mg to 550 mg, e.g., at a dose of about 300 mg or about 400 mg, e.g., once every three weeks (Q3W) or once every four weeks (Q4W), e.g., at a dose of about 300 mg Q3W, or at a dose of about 400 mg Q4W, optionally wherein: the anti-CD73 antibody molecule is administered, e.g., intravenously, at a dose of about 5 mg to 100 mg, e.g., 20 mg, Q2W, and the anti-PD-1 antibody molecule is administered, e.g., intravenously, at a dose of about 350 mg to 450 mg, e.g., 400 mg, Q4W; the anti-CD73 antibody molecule is administered, e.g., intravenously, at a dose of about 5 mg to 100 mg, e.g., 60 mg, Q2W, and the anti-PD-1 antibody molecule is administered, e.g., intravenously, at a dose of about 350 mg to 450 mg, e.g., 400 mg, Q4W; the anti-CD73 antibody molecule is administered, e.g., intravenously, at a dose of about 100 mg to 500 mg, e.g., 200 mg, Q2W, and the anti-PD-1 antibody molecule is administered, e.g., intravenously, at a dose of about 350 mg to 450 mg, e.g., 400 mg, Q4W; the anti-CD73 antibody molecule is administered, e.g., intravenously, at a dose of about 500 mg to 1000 mg, e.g., 600 mg, Q2W, and the anti-PD-1 antibody molecule is administered, e.g., intravenously, at a dose of about 350 mg to 450 mg, e.g., 400 mg, Q4W; the anti-CD73 antibody molecule is administered, e.g., intravenously, at a dose of about 1000 mg to 1500 mg, e.g., 1200 mg, Q2W, and the anti-PD-1 antibody molecule is administered, e.g., intravenously, at a dose of about 350 mg to 450 mg, e.g., 400 mg, Q4W; the anti-CD73 antibody molecule is administered, e.g., intravenously, at a dose of about 2000 mg to 2500 mg, e.g., 2400 mg, Q2W, and the anti-PD-1 antibody molecule is administered, e.g., intravenously, at a dose of about 350 mg to 450 mg, e.g., 400 mg, Q4W; the anti-CD73 antibody molecule is administered, e.g., intravenously, at a dose of about 3000 mg to 3500 mg, e.g., 3000 mg, Q2W, and the anti-PD-1 antibody molecule is administered, e.g., intravenously, at a dose of about 350 mg to 450 mg, e.g., 400 mg, Q4W; or the anti-CD73 antibody molecule is administered, e.g., intravenously, at a dose of about 3500 mg to 4000 mg, e.g., 3600 mg, Q2W, and the anti-PD-1 antibody molecule is administered, e.g., intravenously, at a dose of about 350 mg to 450 mg, e.g., 400 mg, Q4W.

In some embodiments, the therapeutic anti-CD73 antibody molecule is administered in combination with an adenosine A2AR antagonist, optionally wherein: (i) the adenosine A2AR antagonist is selected from the group consisting of PBF509, CPI444, AZD4635, Vipadenant, GBV-2034, and AB928; or (ii) the adenosine A2AR antagonist is selected from the group consisting of 5-bromo-2,6-di-(1H-pyrazol-1-yl)pyrimidine-4-amine; (S)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; (R)-7-(5-methylfuran-2-yl)-3-((6-(((tetrahydrofuran-3-yl)oxy)methyl)pyridin-2-yl)methyl)-3H-[1,2,3]triazolo[4,5-c]pyrimidin-5-amine, or racemate thereof; 7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; and 6-(2-chloro-6-methylpyridin-4-yl)-5-(4-fluorophenyl)-1,2,4-triazin-3-amine, optionally wherein: the adenosine A2AR antagonist is administered, e.g., orally, at a dose of about 20 mg to 60 mg, about 60 mg to 100 mg, about 100 mg to 140 mg, about 140 mg to 180 mg, about 180 mg to 220 mg, about 220 mg to 260 mg, about 260 mg to 300 mg, about 300 mg to 340 mg, about 340 mg to 380 mg, about 380 mg to 480 mg, about 480 mg to 580 mg, or about 580 mg to 680 mg, e.g., at a dose of about 40 mg, about 80 mg, about 160 mg, about 320 mg, about 480 mg, or about 620 mg, e.g., once a day (QD), twice a day (BID), or three times a day (TID), e.g., BID, optionally wherein: the anti-CD73 antibody molecule is administered, e.g., intravenously, at a dose of about 5 mg to 100 mg, e.g., 20 mg, Q2W, and the adenosine A2AR antagonist is administered, e.g., orally, at a dose of about 20 mg to 60 mg, e.g., 40 mg, BID; the anti-CD73 antibody molecule is administered, e.g., intravenously, at a dose of about 5 mg to 100 mg, e.g., 60 mg, Q2W, and the adenosine A2AR antagonist is administered, e.g., orally, at a dose of about 20 mg to 60 mg, e.g., 40 mg, BID; the anti-CD73 antibody molecule is administered, e.g., intravenously, at a dose of about 100 mg to 500 mg, e.g., 200 mg, Q2W, and the adenosine A2AR antagonist is administered, e.g., orally, at a dose of about 60 mg to 100 mg, e.g., 80 mg, BID; the anti-CD73 antibody molecule is administered, e.g., intravenously, at a dose of about 100 mg to 500 mg, e.g., 200 mg, Q2W, and the adenosine A2AR antagonist is administered, e.g., orally, at a dose of about 140 mg to 180 mg, e.g., 160 mg, BID; the anti-CD73 antibody molecule is administered, e.g., intravenously, at a dose of about 500 mg to 1000 mg, e.g., 600 mg, Q2W, and the adenosine A2AR antagonist is administered, e.g., orally, at a dose of about 140 mg to 180 mg, e.g., 160 mg, BID; the anti-CD73 antibody molecule is administered, e.g., intravenously, at a dose of about 1000 mg to 1500 mg, e.g., 1200 mg, Q2W, and the adenosine A2AR antagonist is administered, e.g., orally, at a dose of about 140 mg to 180 mg, e.g., 160 mg, BID; the anti-CD73 antibody molecule is administered, e.g., intravenously, at a dose of about 2000 mg to 2500 mg, e.g., 2400 mg, Q2W, and the adenosine A2AR antagonist is administered, e.g., orally, at a dose of about 140 mg to 180 mg, e.g., 160 mg, BID; the anti-CD73 antibody molecule is administered, e.g., intravenously, at a dose of about 2000 mg to 2500 mg, e.g., 2400 mg, Q2W, and the adenosine A2AR antagonist is administered, e.g., orally, at a dose of about 300 mg to 340 mg, e.g., 320 mg, BID; the anti-CD73 antibody molecule is administered, e.g., intravenously, at a dose of about 2000 mg to 2500 mg, e.g., 2400 mg, Q2W, and the adenosine A2AR antagonist is administered, e.g., orally, at a dose of about 480 mg to 580 mg, e.g., 480 mg, BID; the anti-CD73 antibody molecule is administered, e.g., intravenously, at a dose of about 2000 mg to 2500 mg, e.g., 2400 mg, Q2W, and the adenosine A2AR antagonist is administered, e.g., orally, at a dose of about 580 mg to 680 mg, e.g., 620 mg, BID; the anti-CD73 antibody molecule is administered, e.g., intravenously, at a dose of about 3000 mg to 3500 mg, e.g., 3000 mg, Q2W, and the adenosine A2AR antagonist is administered, e.g., orally, at a dose of about 300 mg to 340 mg, e.g., 320 mg, BID; the anti-CD73 antibody molecule is administered, e.g., intravenously, at a dose of about 3000 mg to 3500 mg, e.g., 3000 mg, Q2W, and the adenosine A2AR antagonist is administered, e.g., orally, at a dose of about 480 mg to 580 mg, e.g., 480 mg, BID; the anti-CD73 antibody molecule is administered, e.g., intravenously, at a dose of about 3000 mg to 3500 mg, e.g., 3000 mg, Q2W, and the adenosine A2AR antagonist is administered, e.g., orally, at a dose of about 580 mg to 680 mg, e.g., 620 mg, BID; the anti-CD73 antibody molecule is administered, e.g., intravenously, at a dose of about 3500 mg to 4000 mg, e.g., 3600 mg, Q2W, and the adenosine A2AR antagonist is administered, e.g., orally, at a dose of about 300 mg to 340 mg, e.g., 320 mg, BID; the anti-CD73 antibody molecule is administered, e.g., intravenously, at a dose of about 3500 mg to 4000 mg, e.g., 3600 mg, Q2W, and the adenosine A2AR antagonist is administered, e.g., orally, at a dose of about 480 mg to 580 mg, e.g., 480 mg, BID; or the anti-CD73 antibody molecule is administered, e.g., intravenously, at a dose of about 3500 mg to 4000 mg, e.g., 3600 mg, Q2W, and the adenosine A2AR antagonist is administered, e.g., orally, at a dose of about 580 mg to 680 mg, e.g., 620 mg, BID.

In some embodiments, the therapeutic anti-CD73 antibody molecule is administered in combination with a PD-1 inhibitor and an adenosine A2AR antagonist, optionally wherein: the PD-1 inhibitor is an anti-PD-1 antibody molecule, wherein the anti-PD-1 antibody molecule is administered, e.g., intravenously, at a dose of about 250 mg to 350 mg, about 350 mg to 450 mg, or about 450 mg to 550 mg, e.g., at a dose of about 300 mg or about 400 mg, e.g., once every three weeks (Q3W) or once every four weeks (Q4W), e.g., at a dose of about 300 mg Q3W, or at a dose of about 400 mg Q4W, optionally wherein: the adenosine A2AR antagonist is administered, e.g., orally, at a dose of about 20 mg to 60 mg, about 60 mg to 100 mg, about 100 mg to 140 mg, about 140 mg to 180 mg, about 180 mg to 220 mg, about 220 mg to 260 mg, about 260 mg to 300 mg, about 300 mg to 340 mg, or about 340 mg to 380 mg, e.g., at a dose of about 40 mg, about 80 mg, about 160 mg, or about 320 mg, e.g., once a day (QD), twice a day (BID), or three times a day (TID), e.g., BID, optionally wherein: the anti-CD73 antibody molecule is administered, e.g., intravenously, at a dose of about 5 mg to 100 mg, e.g., 20 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 20 mg to 60 mg, e.g., 40 mg, BID; the anti-CD73 antibody molecule is administered, e.g., intravenously, at a dose of about 5 mg to 100 mg, e.g., 60 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 20 mg to 60 mg, e.g., 40 mg, BID; the anti-CD73 antibody molecule is administered, e.g., intravenously, at a dose of about 100 mg to 500 mg, e.g., 200 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 60 mg to 100 mg, e.g., 80 mg, BID; the anti-CD73 antibody molecule is administered, e.g., intravenously, at a dose of about 100 mg to 500 mg, e.g., 200 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 140 mg to 180 mg, e.g., 160 mg, BID; the anti-CD73 antibody molecule is administered, e.g., intravenously, at a dose of about 500 mg to 1000 mg, e.g., 600 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 140 mg to 180 mg, e.g., 160 mg, BID; the anti-CD73 antibody molecule is administered, e.g., intravenously, at a dose of about 1000 mg to 1500 mg, e.g., 1200 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 140 mg to 180 mg, e.g., 160 mg, BID; the anti-CD73 antibody molecule is administered, e.g., intravenously, at a dose of about 2000 mg to 2500 mg, e.g., 2400 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 140 mg to 180 mg, e.g., 160 mg, BID; the anti-CD73 antibody molecule is administered, e.g., intravenously, at a dose of about 2000 mg to 2500 mg, e.g., 2400 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 300 mg to 340 mg, e.g., 320 mg, BID; the anti-CD73 antibody molecule is administered, e.g., intravenously, at a dose of about 2000 mg to 2500 mg, e.g., 2400 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 480 mg to 580 mg, e.g., 480 mg, BID; the anti-CD73 antibody molecule is administered, e.g., intravenously, at a dose of about 2000 mg to 2500 mg, e.g., 2400 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 580 mg to 680 mg, e.g., 620 mg, BID; the anti-CD73 antibody molecule is administered, e.g., intravenously, at a dose of about 3000 mg to 3500 mg, e.g., 3000 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 300 mg to 340 mg, e.g., 320 mg, BID; the anti-CD73 antibody molecule is administered, e.g., intravenously, at a dose of about 3000 mg to 3500 mg, e.g., 3000 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 480 mg to 580 mg, e.g., 480 mg, BID; the anti-CD73 antibody molecule is administered, e.g., intravenously, at a dose of about 3000 mg to 3500 mg, e.g., 3000 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 580 mg to 680 mg, e.g., 620 mg, BID; the anti-CD73 antibody molecule is administered, e.g., intravenously, at a dose of about 3500 mg to 4000 mg, e.g., 3600 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 300 mg to 340 mg, e.g., 320 mg, BID; the anti-CD73 antibody molecule is administered, e.g., intravenously, at a dose of about 3500 mg to 4000 mg, e.g., 3600 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 480 mg to 580 mg, e.g., 480 mg, BID; or the anti-CD73 antibody molecule is administered, e.g., intravenously, at a dose of about 3500 mg to 4000 mg, e.g., 3600 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 580 mg to 680 mg, e.g., 620 mg, BID.

In one aspect, this disclosure provides an antibody molecule (e.g., an isolated or recombinant antibody molecule) having one or more (e.g., 2, 3, 4, 5, 6, 7, 8, 9, 10, 11, 12, 13, 14, 15, 16, 17, 18, 19, 20, 21, 22, 23, 24, 25, or more, e.g., all) of the following properties:

(i) binds to CD73, e.g., human CD73, with high affinity, e.g., with a dissociation constant (K_{D}) of less than about 100 nM, e.g., less than about 10 nM, 1 nM, 0.1 nM, or 0.01 nM, e.g., when the antibody molecule is tested as a bivalent antibody molecule using Octet;

(ii) binds to soluble human CD73 or membrane-bound human CD73, or both;

(iii) binds substantially to a non-human primate CD73, e.g., cynomolgus CD73, with a dissociation constant (K_{D}) of less than about 100 nM, e.g., less than about 10 nM, 1 nM, 0.1 nM, or 0.01 nM, e.g., when the antibody molecule is tested as a bivalent antibody molecule using Octet;

(iv) does not bind to murine CD73, e.g., as determined using Octet, e.g., as described in Example 1;

(v) inhibits or reduces the enzymatic activity of CD73 (e.g., soluble human CD73 or membrane-bound human CD73), e.g., inhibits or reduces human CD73 mediated conversion of adenosine monophosphate (AMP) to adenosine, e.g., as measured by a method described herein, e.g., when the antibody molecule is tested as a bivalent antibody in a malachite green (MG) phosphate assay or a modified Cell Titer Glo (CTG) assay, e.g., as described in Example 1;

(vi) increases proliferation of anti-CD3/anti-CD28 stimulated T cells, e.g., CD4+ T cells, in the presence of adenosine monophosphate (AMP), e.g., as measured by a method described herein, e.g., when the antibody molecule is tested as a bivalent antibody in a CellTrace Violet (CTV) cell proliferation assay, e.g., as described in Example 1;

(vii) increases internalization of human CD73 into a cell when bound to human CD73 expressed on the cell surface, e.g., increases internalization of human CD73 into a cell by at least 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold.

(viii) binds to an epitope on CD73, e.g., the same or similar epitope as the epitope recognized by an antibody molecule described herein, e.g., a human anti-CD73 antibody molecule as described herein, e.g., an antibody molecule of Table 1;

(ix) binds the same (or substantially the same) or an overlapping (or substantially overlapping) epitope with a second antibody molecule to CD73, wherein the second antibody molecule is an antibody molecule described herein, e.g., an antibody molecule shown in Table 1;

(x) inhibits, e.g., competitively inhibits, the binding of a second antibody molecule to CD73 wherein the second antibody molecule is an antibody molecule described herein, e.g., an antibody molecule shown in Table 1;

(xi) shows the same or similar binding affinity or specificity, or both, as an antibody molecule described in Table 1, e.g., an antibody molecule comprising a heavy chain variable region and/or a light chain variable region shown in Table 1;

(xii) binds to the N-terminal domain of human CD73;

(xiii) binds to the A-loop and/or B-loop of human CD73;

(xiv) reduces hydrogen-deuterium exchange at one or more regions of a protein comprising the amino acid sequence of residues 27-547 of SEQ ID NO: 105 (e.g., a protein consisting of the amino acid sequence of SEQ ID NO: 171) when bound thereto, wherein the one or more regions are selected from the group consisting of residues 158-172, residues 206-215, residues 368-387, and residues 87-104 of SEQ ID NO: 105, e.g., when the antibody molecule is tested as a bivalent antibody molecule using hydrogen deuterium-exchange mass spectrometry;

(xv) when bound to a protein comprising the amino acid sequence of residues 27-547 of SEQ ID NO: 105 (e.g., a protein consisting of the amino acid sequence of SEQ ID NO: 171), induces a conformational change in residues 368-387 of SEQ ID NO: 105;

(xvi) contacts, e.g., directly or indirectly, at least one, two, three, or four residues within residues 158-172 of SEQ ID NO: 105;

(xvii) contacts, e.g., directly or indirectly, at least one, two, three, four or five residues within residues 206-215 of SEQ ID NO: 105;

(xviii) contacts, e.g., directly or indirectly, at least one, two, three, four or five residues within residues 368-387 of SEQ ID NO: 105 or 106;

(xix) contacts, e.g., directly or indirectly, at least one, two, three, four or five residues within residues 87-104 of SEQ ID NO: 105;

(xx) binds to a human CD73 dimer, said dimer consisting of a first CD73 monomer and a second CD73 monomer, wherein when the antibody molecule comprises a first antigen binding domain and a second antigen binding domain, the first antigen binding domain binds to the first CD73 monomer and the second antigen binding domain binds to the second CD73 monomer, e.g., when tested using size exclusion chromatography;

(xxi) preferentially binds to an open conformation, e.g., a catalytically inactive conformation, of CD73 over a closed conformation, e.g., a catalytically active conformation, of CD73, e.g., does not bind to or binds to the closed conformation, e.g., the catalytically active conformation, of CD73 with lower affinity, e.g., 50%, 60%, 70%, 80%, 90%, 95%, or 99% lower affinity than when the antibody molecule binds to the open conformation, e.g., the catalytically inactive conformation, of CD73;

(xxii) locks human CD73 in a catalytically inactive open conformation;

(xxiii) prevents or reduces the conversion of human CD73 from a catalytically inactive open conformation to a catalytically active closed conformation, e.g., reduces the conversion by at least 1.5-fold, 2-fold, 5-fold, 10-fold, 20-fold, 30-fold, 40-fold, 50-fold, 60-fold, 70-fold, 80-fold, 90-fold, or 100-fold;

(xxiv) has one or more biological properties of an antibody molecule described herein, e.g., an antibody molecule shown in Table 1;

(xxv) has one or more pharmacokinetic properties of an antibody molecule described herein, e.g., an antibody molecule shown in Table 1; or

(xxvi) modulates (e.g., inhibits) one or more activities of CD73, e.g., results in one or more of: inhibiting or reducing the enzymatic activity of CD73; inhibiting or reducing the conversion of adenosine monophosphate (AMP) to adenosine; increasing proliferation of anti-CD3/anti-CD28 stimulated T cells, e.g., CD4+ T cells, in the presence of adenosine monophosphate (AMP); inhibiting proliferation of regulatory T cells; increasing effector T cell responses; and/or inhibiting migration, infiltration, or expansion of myeloid derived suppressor cells.

In one aspect, disclosed herein is an antibody molecule that binds to a human CD73 dimer, said dimer consisting of a first CD73 monomer and a second CD73 monomer, wherein when the antibody molecule comprises a first antigen binding domain and a second antigen binding domain, the first antigen binding domain binds to the first CD73 monomer and the second antigen binding domain binds to the second CD73 monomer, e.g., when tested using size exclusion chromatography. In one embodiment, the first or second CD73 monomer comprises the amino acid sequence of residues 27-547 of SEQ ID NO: 105. In one embodiment, the first or second CD73 monomer consists of the amino acid sequence of SEQ ID NO: 171.

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein when the antibody molecules in the plurality each comprises the same first antigen binding domain and the same second antigen binding domain, at least 30%, 35%, or 40% of the antibody molecules in said composition bind to the CD73 dimer to form a complex, wherein each of said complex consists of one antibody molecule and one CD73 dimer, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2. In one embodiment, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein when the antibody molecules in the plurality each comprises the same first antigen binding domain and the same second antigen binding domain, at least 30%, 35%, or 40% of the antibody molecules in said composition bind to the CD73 dimer to form a complex, wherein each of said complex consists of one antibody molecule and one CD73 dimer, e.g., when measured using a size exclusion chromatography (SEC) assay comprising the following steps:

(i) incubating a sample containing an equimolar amount of the bivalent antibody molecule and the CD73 monomer overnight at 4° C.;

(ii) running the sample through a SEC column (e.g., a Shodex Protein KW-803 column (8×300 mm ID)) at room temperature in a buffer containing 90% 2×PBS and 10% isopropanol by volume; and

(iii) analyzing SEC peaks to obtain the relative percentage values of distinguishable antibody CD73 species by determining the amount of the antibody molecules in the complex relative to the total amount of the antibody molecules binding to CD73 (excluding unbound antibody molecules).

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein when the antibody molecules in the plurality each comprises the same first antigen binding domain and the same second antigen binding domain, at least 70%, 75%, or 80% of the antibody molecules in said composition bind to the CD73 dimer to form a complex, wherein each of said complex consists of one antibody molecule and one CD73 dimer, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2. In one embodiment, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein when the antibody molecules in the plurality each comprises the same first antigen binding domain and the same second antigen binding domain, at least 70%, 75%, or 80% of the antibody molecules in said composition bind to the CD73 dimer to form a complex, wherein each of said complex consists of one antibody molecule and one CD73 dimer, e.g., when measured using a size exclusion chromatography (SEC) assay comprising the following steps:

(i) incubating a sample containing an equimolar amount of the bivalent antibody molecule and the CD73 monomer overnight at 4° C.;

(ii) running the sample through a SEC column (e.g., a Shodex Protein KW-803 column (8×300 mm ID)) at room temperature in a buffer containing 90% 2×PBS and 10% isopropanol by volume; and

(iii) analyzing SEC peaks to obtain the relative percentage values of distinguishable antibody CD73 species by determining the amount of the antibody molecules in the complex relative to the total amount of the antibody molecules binding to CD73 (excluding unbound antibody molecules).

In one aspect, disclosed herein is a plurality of an antibody molecule that binds to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein when the antibody molecules in the plurality each comprises the same first antigen binding domain and the same second antigen binding domain, at most 60%, 65%, or 70% of the antibody molecules in said composition bind to the CD73 dimer to form a complex, wherein each of said complex comprises two or more antibody molecules and two or more CD73 dimers, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2. In one embodiment, disclosed herein is a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein when the antibody molecules in the plurality each comprises the same first antigen binding domain and the same second antigen binding domain, at most 60%, 65%, or 70% of the antibody molecules in said composition bind to the CD73 dimer to form a complex, wherein each of said complex comprises two or more antibody molecules and two or more CD73 dimers, e.g., when measured using a size exclusion chromatography (SEC) assay comprising the following steps:

(i) incubating a sample containing an equimolar amount of the bivalent antibody molecule and the CD73 monomer overnight at 4° C.;

(ii) running the sample through a SEC column (e.g., a Shodex Protein KW-803 column (8×300 mm ID)) at room temperature in a buffer containing 90% 2×PBS and 10% isopropanol by volume; and

(iii) analyzing SEC peaks to obtain the relative percentage values of distinguishable antibody CD73 species by determining the amount of the antibody molecules in the complex relative to the total amount of the antibody molecules binding to CD73 (excluding unbound antibody molecules).

In one aspect, disclosed herein is a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein when the antibody molecules in the plurality each comprises the same first antigen binding domain and the same second antigen binding domain, at most 20%, 25%, or 30% of the antibody molecules in said composition bind to the CD73 dimer to form a complex, wherein each of said complex comprises two or more antibody molecules and two or more CD73 dimers, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2. In one embodiment, disclosed herein is a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein when the antibody molecules in the plurality each comprises the same first antigen binding domain and the same second antigen binding domain, at most 20%, 25%, or 30% of the antibody molecules in said composition bind to the CD73 dimer to form a complex, wherein each of said complex comprises two or more antibody molecules and two or more CD73 dimers, e.g., when measured using a size exclusion chromatography (SEC) assay comprising the following steps:

(i) incubating a sample containing an equimolar amount of the bivalent antibody molecule and the CD73 monomer overnight at 4° C.;

(ii) running the sample through a SEC column (e.g., a Shodex Protein KW-803 column (8×300 mm ID)) at room temperature in a buffer containing 90% 2×PBS and 10% isopropanol by volume; and

(iii) analyzing SEC peaks to obtain the relative percentage values of distinguishable antibody CD73 species by determining the amount of the antibody molecules in the complex relative to the total amount of the antibody molecules binding to CD73 (excluding unbound antibody molecules).

In one aspect, disclosed herein is an antibody molecule that binds to human CD73, the antibody molecule preferentially binds to an open conformation, e.g., a catalytically inactive conformation, of CD73 over a closed conformation, e.g., a catalytically active conformation, of CD73, e.g., does not bind to or binds to the closed conformation, e.g., the catalytically active conformation, of CD73 with lower affinity, e.g., 50%, 60%, 70%, 80%, 90%, 95%, or 99% lower affinity than when the antibody molecule binds to the open conformation, e.g., the catalytically inactive conformation, of CD73.

In one aspect, disclosed herein is an antibody molecule that binds to human CD73, wherein the antibody molecule prevents or reduces the conversion of human CD73 from a catalytically inactive open conformation to a catalytically active closed conformation, e.g., reduces the conversion by at least 1.5-fold, 2-fold, 5-fold, 10-fold, 20-fold, 30-fold, 40-fold, 50-fold, 60-fold, 70-fold, 80-fold, 90-fold, or 100-fold, compared to the conversion in the absence of the antibody molecule.

In one aspect, disclosed herein is an antibody molecule that binds to human CD73, wherein the antibody molecule reduces hydrogen-deuterium exchange at one or more regions of a protein comprising the amino acid sequence of residues 27-547 of SEQ ID NO: 105 (e.g., a protein consisting of the amino acid sequence of SEQ ID NO: 171) when bound thereto, wherein the one or more regions are selected from the group consisting of residues 158-172, residues 206-215, residues 368-387, and residues 87-104 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature.

In one aspect, disclosed herein is an antibody molecule that binds to human CD73, wherein the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at X_{C} residue(s) selected from core region C (residues 368-387 of SEQ ID NO: 105) to a greater extent than at X_{A} residue(s) selected from core region A (residues 158-172 of SEQ ID NO: 105), X_{B} residue(s) selected from core region B (residues 206-215 of SEQ ID NO: 105), or X_{D} residue(s) selected from core region D (residues 297-309 of SEQ ID NO: 105), e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature, wherein:

X_{C} is equal to 1, 2, 3, 4, 5, 6, 7, 8, 9, 10, 15, or 20,
X_{A} is equal to 1, 2, 3, 4, 5, 6, 7, 8, 9, 10, 11, 12, 13, or 14,
X_{B} is equal to 1, 2, 3, 4, 5, 6, 7, 8, 9, or 10, and
X_{D} is equal to 1, 2, 3, 4, 5, 6, 7, 8, 9, 10, 11, 12, or 13.

In one embodiment, the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at 20 residues selected from core region C to a greater extent than at:

(i) 14 residues selected from core region A,
(ii) 10 residues selected from core region B, or
(iii) 13 residues selected from core region D,
e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature. In one embodiment, antibody binding reduces the average hydrogen-deuterium exchange at core region C to a greater extent than at core region A. In one embodiment, antibody binding reduces the average hydrogen-deuterium exchange at core region C to a greater extent than at core region B. In one embodiment, antibody binding reduces the average hydrogen-deuterium exchange at core region C to a greater extent than at core region D. In one embodiment, antibody binding reduces the average hydrogen-deuterium exchange at core region C to a greater extent than at core region A and core region B. In one embodiment, antibody binding reduces the average hydrogen-deuterium exchange at core region C to a greater extent than at core region A and core region D. In one embodiment, antibody binding reduces the average hydrogen-deuterium exchange at core region C to a greater extent than at core region B and core region D. In one embodiment, antibody binding reduces the average hydrogen-deuterium exchange at core region C to a greater extent than at core region A, core region B, and core region D.

In one embodiment, the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at 15 residues selected from core region C to a greater extent than at:

(i) 10 residues selected from core region A,
(ii) 8 residues selected from core region B, or
(iii) 10 residues selected from core region D,
e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature. In one embodiment, antibody binding reduces the average hydrogen-deuterium exchange at core region C to a greater extent than at core region A. In one embodiment, antibody binding reduces the average hydrogen-deuterium exchange at core region C to a greater extent than at core region B. In one embodiment, antibody binding reduces the average hydrogen-deuterium exchange at core region C to a greater extent than at core region D. In one embodiment, antibody binding reduces the average hydrogen-deuterium exchange at core region C to a greater extent than at core region A and core region B. In one embodiment, antibody binding reduces the average hydrogen-deuterium exchange at core region C to a greater extent than at core region A and core region D. In one embodiment, antibody binding reduces the average hydrogen-deuterium exchange at core region C to a greater extent than at core region B and core region D. In one embodiment, antibody binding reduces the average hydrogen-deuterium exchange at core region C to a greater extent than at core region A, core region B, and core region D.

In one embodiment, the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at 10 residues selected from core region C to a greater extent than at:

(i) 7 residues selected from core region A,
(ii) 5 residues selected from core region B, or
(iii) 7 residues selected from core region D,
e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature. In one embodiment, antibody binding reduces the average hydrogen-deuterium exchange at core region C to a greater extent than at core region A. In one embodiment, antibody binding reduces the average hydrogen-deuterium exchange at core region C to a greater extent than at core region B. In one embodiment, antibody binding reduces the average hydrogen-deuterium exchange at core region C to a greater extent than at core region D. In one embodiment, antibody binding reduces the average hydrogen-deuterium exchange at core region C to a greater extent than at core region A and core region B. In one embodiment, antibody binding reduces the average hydrogen-deuterium exchange at core region C to a greater extent than at core region A and core region D. In one embodiment, antibody binding reduces the average hydrogen-deuterium exchange at core region C to a greater extent than at core region B and core region D. In one embodiment, antibody binding reduces the average hydrogen-deuterium exchange at core region C to a greater extent than at core region A, core region B, and core region D.

In one aspect, disclosed herein is an antibody molecule that binds to human CD73, wherein the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 368-387 of SEQ ID NO: 105 to a greater extent than at residues 158-172, residues 206-215, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature. In one embodiment, antibody binding reduces the average hydrogen-deuterium exchange at residues 368-387 of SEQ ID NO: 105 to a greater extent than at residues 158-172 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105. In one embodiment, antibody binding reduces the average hydrogen-deuterium exchange at residues 368-387 of SEQ ID NO: 105 to a greater extent than at residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 206-215 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105, residues 206-215 of SEQ ID NO: 105, and residues 297-309 of SEQ ID NO: 105.

In one aspect, disclosed herein is an antibody molecule that binds to human CD73, wherein the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at X_{A} residue(s) selected from core region A (residues 158-172 of SEQ ID NO: 105) to a greater extent than at X_{B} residue(s) selected from core region B (residues 206-215 of SEQ ID NO: 105), X_{C} residue(s) selected from core region C (residues 368-387 of SEQ ID NO: 105), or X_{D} residue(s) selected from core region D (residues 297-309 of SEQ ID NO: 105), e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature, wherein:

X_{A} is equal to 1, 2, 3, 4, 5, 6, 7, 8, 9, 10, 11, 12, 13, or 14,
X_{B} is equal to 1, 2, 3, 4, 5, 6, 7, 8, 9, or 10,
X_{C} is equal to 1, 2, 3, 4, 5, 6, 7, 8, 9, 10, 15, or 20, and
X_{D} is equal to 1, 2, 3, 4, 5, 6, 7, 8, 9, 10, 11, 12, or 13.

In one embodiment, the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at 14 residues selected from core region A to a greater extent than at:

(i) 10 residues selected from core region B,
(ii) 20 residues selected from core region C, or
(iii) 13 residues selected from core region D,
e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature. In one embodiment, antibody binding reduces the average hydrogen-deuterium exchange at core region A to a greater extent than at core region B. In one embodiment, antibody binding reduces the average hydrogen-deuterium exchange at core region A to a greater extent than at core region C. In one embodiment, antibody binding reduces the average hydrogen-deuterium exchange at core region A to a greater extent than at core region D. In one embodiment, antibody binding reduces the average hydrogen-deuterium exchange at core region A to a greater extent than at core region B and core region C. In one embodiment, antibody binding reduces the average hydrogen-deuterium exchange at core region A to a greater extent than at core region B and core region D. In one embodiment, antibody binding reduces the average hydrogen-deuterium exchange at core region A to a greater extent than at core region C and core region D. In one embodiment, antibody binding reduces the average hydrogen-deuterium exchange at core region A to a greater extent than at core region B, core region C, and core region D.

In one embodiment, the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at 10 residues selected from core region A to a greater extent than at:

(i) 8 residues selected from core region B,
(ii) 15 residues selected from core region C, or
(iii) 10 residues selected from core region D, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature. In one embodiment, antibody binding reduces the average hydrogen-deuterium exchange at core region A to a greater extent than at core region B. In one embodiment, antibody binding reduces the average hydrogen-deuterium exchange at core region A to a greater extent than at core region C. In one embodiment, antibody binding reduces the average hydrogen-deuterium exchange at core region A to a greater extent than at core region D. In one embodiment, antibody binding reduces the average hydrogen-deuterium exchange at core region A to a greater extent than at core region B and core region C. In one embodiment, antibody binding reduces the average hydrogen-deuterium exchange at core region A to a greater extent than at core region B and core region D. In one embodiment, antibody binding reduces the average hydrogen-deuterium exchange at core region A to a greater extent than at core region C and core region D. In one embodiment, antibody binding reduces the average hydrogen-deuterium exchange at core region A to a greater extent than at core region B, core region C, and core region D.

In one embodiment, the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at 7 residues selected from core region A to a greater extent than at:

(i) 5 residues selected from core region B,
(ii) 10 residues selected from core region C, or
(iii) 7 residues selected from core region D,
e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature. In one embodiment, antibody binding reduces the average hydrogen-deuterium exchange at core region A to a greater extent than at core region B. In one embodiment, antibody binding reduces the average hydrogen-deuterium exchange at core region A to a greater extent than at core region C. In one embodiment, antibody binding reduces the average hydrogen-deuterium exchange at core region A to a greater extent than at core region D. In one embodiment, antibody binding reduces the average hydrogen-deuterium exchange at core region A to a greater extent than at core region B and core region C. In one embodiment, antibody binding reduces the average hydrogen-deuterium exchange at core region A to a greater extent than at core region B and core region D. In one embodiment, antibody binding reduces the average hydrogen-deuterium exchange at core region A to a greater extent than at core region C and core region D. In one embodiment, antibody binding reduces the average hydrogen-deuterium exchange at core region A to a greater extent than at core region B, core region C, and core region D.

In one aspect, disclosed herein is an antibody molecule that binds to human CD73, wherein the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 158-172 of SEQ ID NO: 105 to a greater extent than at residues 206-215 of SEQ ID NO: 105, residues 368-387 of SEQ ID NO: 105, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature. In one embodiment, antibody binding reduces the average hydrogen-deuterium exchange at residues 158-172 of SEQ ID NO: 105 to a greater extent than at residues 206-215 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105. In one embodiment, antibody binding reduces the average hydrogen-deuterium exchange at residues 158-172 of SEQ ID NO: 105 to a greater extent than at residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 368-387 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105, residues 368-387 of SEQ ID NO: 105, and residues 297-309 of SEQ ID NO: 105.

In one aspect, disclosed herein is an antibody molecule that binds to human CD73, wherein the antibody molecule reduces hydrogen-deuterium exchange at one or more regions of a protein comprising the amino acid sequence of residues 27-547 of SEQ ID NO: 105 (e.g., a protein consisting of the amino acid sequence of SEQ ID NO: 171) when bound thereto, wherein the one or more regions are selected from the group consisting of residues 158-172, residues 206-215, residues 368-387, and residues 87-104 of SEQ ID NO: 105, wherein the region having the greatest reduction in the average hydrogen-deuterium exchange among the one or more regions is not residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature.

In one aspect, disclosed herein is an antibody molecule that binds to human CD73, wherein the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of more than, e.g., 0.02, 0.03, 0.04, 0.05, or 0.06 Da per residue at residues 368-387 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature.

In one aspect, disclosed herein is an antibody molecule that binds to human CD73, wherein the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of less than, e.g., 0.05, 0.04, 0.03, or 0.02 Da per residue at residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature.

In one aspect, disclosed herein is an antibody molecule that binds to human CD73, wherein the antibody molecule contacts, e.g., directly or indirectly, at least one, two, three, or four residues within residues 158-172 of SEQ ID NO: 105. In one aspect, disclosed herein is an antibody molecule that binds to human CD73, wherein the antibody molecule contacts, e.g., directly or indirectly, at least one, two, three, four or five residues within residues 206-215 of SEQ ID NO: 105. In one aspect, disclosed herein is an antibody molecule that binds to human CD73, wherein the antibody molecule contacts, e.g., directly or indirectly, at least one, two, three, four or five residues within residues 368-387 of SEQ ID NO: 105 or 106. In one aspect, disclosed herein is an antibody molecule that binds to human CD73, wherein the antibody molecule contacts, e.g., directly or indirectly, at least one, two, three, four or five residues within residues 87-104 of SEQ ID NO: 105.

In one embodiment, the antibody molecule binds to at least one, two, three, or four residues within residues 158-172 of SEQ ID NO: 105. In one embodiment, the antibody molecule binds to at least one, two, three, four or five residues within residues 206-215 of SEQ ID NO: 105.

In one aspect, disclosed herein is an antibody molecule that binds to human CD73, wherein the antibody molecule comprises at least one, two, three, four, five or six complementarity determining regions (CDRs) (or collectively all of the CDRs) from a heavy variable region and/or a light chain variable region comprising an amino acid sequence shown in Table 1 (e.g., from the heavy and light chain variable region sequences of an antibody disclosed in Table 1, e.g., the antibody 918, 350, 356, 358, 930, 373, 374, 376, 377, or 379), or encoded by a nucleotide sequence shown in Table 1. In some embodiments, the CDRs are according to the Kabat definition (e.g., as set out in Table 1). In some embodiments, the CDRs are according to the Chothia definition (e.g., as set out in Table 1). In some embodiments, the CDRs are according to the combined definition based on the Kabat definition and the Chothia definition (e.g., as set out in Table 1). In some embodiments, the CDRs are according to the IMGT definition (e.g., as set out in Table 1). In one embodiment, one or more of the CDRs (or collectively all of the CDRs) have one, two, three, four, five, six or more changes, e g, amino acid substitutions (e.g., conservative amino acid substitutions) or deletions, relative to an amino acid sequence shown in Table 1, or encoded by a nucleotide sequence shown in Table 1.

In one aspect, disclosed herein is an antibody molecule that binds to human CD73, comprising a heavy chain variable region (VH) comprising a heavy chain complementarity determining region 3 (VHCDR3) amino acid sequence of GGLYGSGSYLSDFDL (SEQ ID NO: 37).

In one aspect, disclosed herein is an antibody molecule that binds to human CD73, comprising:

(i) a heavy chain variable region (VH) comprising one, two, or three of: a heavy chain complementarity determining region 1 (VHCDR1) amino acid sequence of X₁X₂AMS (SEQ ID NO: 88), wherein X₁ is R, Y, or S, and X₂ is Y or N; a VHCDR2 amino acid sequence of X₁IX₂GX₃GX₄X₅TYYADSVKG (SEQ ID NO: 89), wherein X₁ is A or S, X₂ is S or T, X₃ is S or T, X₄ is M, G, or S, and X₅ is N, S, L, or Y; and a VHCDR3 amino acid sequence of GGLYGSGSYLSDFDL (SEQ ID NO: 37); and/or

(ii) a light chain variable region (VL) comprising one, two, or three of: a light chain complementarity determining region 1 (VLCDR1) amino acid sequence of RASQSVGSNLA (SEQ ID NO: 48); a VLCDR2 amino acid sequence of GASTRAT (SEQ ID NO: 49); and a VLCDR3 amino acid sequence of QQHNAFPYT (SEQ ID NO: 50).

In some embodiments, the anti-CD73 antibody molecule comprises:

(i) a VH comprising one, two, or three of: a VHCDR1 amino acid sequence of SEQ ID NO: 38, a VHCDR2 amino acid sequence of SEQ ID NO: 36, and a VHCDR3 amino acid sequence of SEQ ID NO: 37 (or a sequence having at least about 85%, 90%, 95%, or 99% sequence identity thereto, and/or having one, two, three or more substitutions, insertions or deletions, e.g., conserved substitutions); and a VL comprising one, two, or three of: a VLCDR1 amino acid sequence of SEQ ID NO: 48, a VLCDR2 amino acid sequence of SEQ ID NO: 49, and a VLCDR3 amino acid sequence of SEQ ID NO: 50 (or a sequence having at least about 85%, 90%, 95%, or 99% sequence identity thereto, and/or having one, two, three or more substitutions, insertions or deletions, e.g., conserved substitutions);

(ii) a VH comprising one, two, or three of: a VHCDR1 amino acid sequence of SEQ ID NO: 72, a VHCDR2 amino acid sequence of SEQ ID NO: 71, and a VHCDR3 amino acid sequence of SEQ ID NO: 37 (or a sequence having at least about 85%, 90%, 95%, or 99% sequence identity thereto, and/or having one, two, three or more substitutions, insertions or deletions, e.g., conserved substitutions); and a VL comprising one, two, or three of: a VLCDR1 amino acid sequence of SEQ ID NO: 48, a VLCDR2 amino acid sequence of SEQ ID NO: 49, and a VLCDR3 amino acid sequence of SEQ ID NO: 50 (or a sequence having at least about 85%, 90%, 95%, or 99% sequence identity thereto, and/or having one, two, three or more substitutions, insertions or deletions, e.g., conserved substitutions);

(iii) a VH comprising one, two, or three of: a VHCDR1 amino acid sequence of SEQ ID NO: 38, a VHCDR2 amino acid sequence of SEQ ID NO: 71, and a VHCDR3 amino acid sequence of SEQ ID NO: 37 (or a sequence having at least about 85%, 90%, 95%, or 99% sequence identity thereto, and/or having one, two, three or more substitutions, insertions or deletions, e.g., conserved substitutions); and a VL comprising one, two, or three of: a VLCDR1 amino acid sequence of SEQ ID NO: 48, a VLCDR2 amino acid sequence of SEQ ID NO: 49, and a VLCDR3 amino acid sequence of SEQ ID NO: 50 (or a sequence having at least about 85%, 90%, 95%, or 99% sequence identity thereto, and/or having one, two, three or more substitutions, insertions or deletions, e.g., conserved substitutions);

(iv) a VH comprising one, two, or three of: a VHCDR1 amino acid sequence of SEQ ID NO: 137, a VHCDR2 amino acid sequence of SEQ ID NO: 136, and a VHCDR3 amino acid sequence of SEQ ID NO: 37 (or a sequence having at least about 85%, 90%, 95%, or 99% sequence identity thereto, and/or having one, two, three or more substitutions, insertions or deletions, e.g., conserved substitutions); and a VL comprising one, two, or three of: a VLCDR1 amino acid sequence of SEQ ID NO: 48, a VLCDR2 amino acid sequence of SEQ ID NO: 49, and a VLCDR3 amino acid sequence of SEQ ID NO: 50 (or a sequence having at least about 85%, 90%, 95%, or 99% sequence identity thereto, and/or having one, two, three or more substitutions, insertions or deletions, e.g., conserved substitutions);

(v) a VH comprising one, two, or three of: a VHCDR1 amino acid sequence of SEQ ID NO: 137, a VHCDR2 amino acid sequence of SEQ ID NO: 146, and a VHCDR3 amino acid sequence of SEQ ID NO: 37 (or a sequence having at least about 85%, 90%, 95%, or 99% sequence identity thereto, and/or having one, two, three or more substitutions, insertions or deletions, e.g., conserved substitutions); and a VL comprising one, two, or three of: a VLCDR1 amino acid sequence of SEQ ID NO: 48, a VLCDR2 amino acid sequence of SEQ ID NO: 49, and a VLCDR3 amino acid sequence of SEQ ID NO: 50 (or a sequence having at least about 85%, 90%, 95%, or 99% sequence identity thereto, and/or having one, two, three or more substitutions, insertions or deletions, e.g., conserved substitutions); or

(vi) a VH comprising one, two, or three of: a VHCDR1 amino acid sequence of SEQ ID NO: 137, a VHCDR2 amino acid sequence of SEQ ID NO: 154, and a VHCDR3 amino acid sequence of SEQ ID NO: 37 (or a sequence having at least about 85%, 90%, 95%, or 99% sequence identity thereto, and/or having one, two, three or more substitutions, insertions or deletions, e.g., conserved substitutions); and a VL comprising one, two, or three of: a VLCDR1 amino acid sequence of SEQ ID NO: 48, a VLCDR2 amino acid sequence of SEQ ID NO: 49, and a VLCDR3 amino acid sequence of SEQ ID NO: 50 (or a sequence having at least about 85%, 90%, 95%, or 99% sequence identity thereto, and/or having one, two, three or more substitutions, insertions or deletions, e.g., conserved substitutions).

In some embodiments, the anti-CD73 antibody molecule comprises a heavy chain variable region comprising the amino acid sequence of SEQ ID NO: 44, 77, 84, 142, 151, or 159, or an amino acid sequence having at least about 85%, 90%, 95%, or 99% sequence identity to SEQ ID NO: 44, 77, 84, 142, 151, or 159.

In some embodiments, the anti-CD73 antibody molecule comprises a light chain variable region comprising the amino acid sequence of SEQ ID NO: 55, or an amino acid sequence having at least about 85%, 90%, 95%, or 99% sequence identity to SEQ ID NO: 55.

In some embodiments, the anti-CD73 antibody molecule comprises a heavy chain comprising the amino acid sequence of SEQ ID NO: 46, 79, 86, 114, 116, or 117, or an amino acid sequence having at least about 85%, 90%, 95%, or 99% sequence identity to SEQ ID NO: 46, 79, 86, 114, 116, or 117.

In some embodiments, the anti-CD73 antibody molecule comprises a light chain comprising the amino acid sequence of SEQ ID NO: 57, or an amino acid sequence having at least about 85%, 90%, 95%, or 99% sequence identity to SEQ ID NO: 57.

In some embodiments, the anti-CD73 antibody molecule comprises:

(i) a heavy chain variable region comprising the amino acid sequence of SEQ ID NO: 44 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto);

(ii) a heavy chain variable region comprising the amino acid sequence of SEQ ID NO: 77 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto);

(iii) a heavy chain variable region comprising the amino acid sequence of SEQ ID NO: 84 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto);

(iv) a heavy chain variable region comprising the amino acid sequence of SEQ ID NO: 142 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto);

(v) a heavy chain variable region comprising the amino acid sequence of SEQ ID NO: 151 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto); or

(vi) a heavy chain variable region comprising the amino acid sequence of SEQ ID NO: 159 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto).

In some embodiments, the anti-CD73 antibody molecule comprises:

(i) a heavy chain comprising the amino acid sequence of SEQ ID NO: 46 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto);

(ii) a heavy chain comprising the amino acid sequence of SEQ ID NO: 114 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto);

(iii) a heavy chain comprising the amino acid sequence of SEQ ID NO: 79 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto);

(iv) a heavy chain comprising the amino acid sequence of SEQ ID NO: 116 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto);

(v) a heavy chain comprising the amino acid sequence of SEQ ID NO: 86 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto); or

(vi) a heavy chain comprising the amino acid sequence of SEQ ID NO: 117 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto).

In one aspect, disclosed herein is an antibody molecule that binds to human CD73, comprising a heavy chain variable region (VH) comprising a VHCDR3 amino acid sequence of ESQESPYNNWFDP (SEQ ID NO: 3).

In one aspect, disclosed herein is an antibody molecule that binds to human CD73, comprising: (i) a heavy chain variable region (VH) comprising a heavy chain complementarity determining region 1 (VHCDR1) amino acid sequence of X₁X₂YWS (SEQ ID NO: 90), wherein X₁ is R, G, or S, and X₂ is Y or R; a VHCDR2 amino acid sequence of YIYX₁X₂GSTX₃YNPSLKS (SEQ ID NO: 91), wherein X₁ is G or S, X₂ is R, S, or T, and X₃ is N or K; and a VHCDR3 amino acid sequence of ESQESPYNNWFDP (SEQ ID NO: 3); and/or

(ii) a light chain variable region (VL) comprising a light chain complementarity determining region 1 (VLCDR1) amino acid sequence of RASQGISSWLA (SEQ ID NO: 14); a VLCDR2 amino acid sequence of AASSLQS (SEQ ID NO: 15); and a VLCDR3 amino acid sequence of QQGNSFPRT (SEQ ID NO: 16).

In some embodiments, the anti-CD73 antibody molecule comprises:

(i) a VH comprising one, two, or three of: a VHCDR1 amino acid sequence of SEQ ID NO: 61, a VHCDR2 amino acid sequence of SEQ ID NO: 60, and a VHCDR3 amino acid sequence of SEQ ID NO: 3 (or a sequence having at least about 85%, 90%, 95%, or 99% sequence identity thereto, and/or having one, two, three or more substitutions, insertions or deletions, e.g., conserved substitutions); and a VL comprising one, two, or three of: a VLCDR1 amino acid sequence of SEQ ID NO: 14, a VLCDR2 amino acid sequence of SEQ ID NO: 15, and a VLCDR3 amino acid sequence of SEQ ID NO: 16 (or a sequence having at least about 85%, 90%, 95%, or 99% sequence identity thereto, and/or having one, two, three or more substitutions, insertions or deletions, e.g., conserved substitutions);

(ii) a VH comprising one, two, or three of: a VHCDR1 amino acid sequence of SEQ ID NO: 4, a VHCDR2 amino acid sequence of SEQ ID NO: 26, and a VHCDR3 amino acid sequence of SEQ ID NO: 3 (or a sequence having at least about 85%, 90%, 95%, or 99% sequence identity thereto, and/or having one, two, three or more substitutions, insertions or deletions, e.g., conserved substitutions); and a VL comprising one, two, or three of: a VLCDR1 amino acid sequence of SEQ ID NO: 14, a VLCDR2 amino acid sequence of SEQ ID NO: 15, and a VLCDR3 amino acid sequence of SEQ ID NO: 16 (or a sequence having at least about 85%, 90%, 95%, or 99% sequence identity thereto, and/or having one, two, three or more substitutions, insertions or deletions, e.g., conserved substitutions);

(iii) a VH comprising one, two, or three of: a VHCDR1 amino acid sequence of SEQ ID NO: 4, a VHCDR2 amino acid sequence of SEQ ID NO: 2, and a VHCDR3 amino acid sequence of SEQ ID NO: 3 (or a sequence having at least about 85%, 90%, 95%, or 99% sequence identity thereto, and/or having one, two, three or more substitutions, insertions or deletions, e.g., conserved substitutions); and a VL comprising one, two, or three of: a VLCDR1 amino acid sequence of SEQ ID NO: 14, a VLCDR2 amino acid sequence of SEQ ID NO: 15, and a VLCDR3 amino acid sequence of SEQ ID NO: 16 (or a sequence having at least about 85%, 90%, 95%, or 99% sequence identity thereto, and/or having one, two, three or more substitutions, insertions or deletions, e.g., conserved substitutions); or

(iv) a VH comprising one, two, or three of: a VHCDR1 amino acid sequence of SEQ ID NO: 163, a VHCDR2 amino acid sequence of SEQ ID NO: 162, and a VHCDR3 amino acid sequence of SEQ ID NO: 3 (or a sequence having at least about 85%, 90%, 95%, or 99% sequence identity thereto, and/or having one, two, three or more substitutions, insertions or deletions, e.g., conserved substitutions); and a VL comprising one, two, or three of: a VLCDR1 amino acid sequence of SEQ ID NO: 14, a VLCDR2 amino acid sequence of SEQ ID NO: 15, and a VLCDR3 amino acid sequence of SEQ ID NO: 16 (or a sequence having at least about 85%, 90%, 95%, or 99% sequence identity thereto, and/or having one, two, three or more substitutions, insertions or deletions, e.g., conserved substitutions).

In some embodiments, the anti-CD73 antibody molecule comprises a heavy chain variable region comprising the amino acid sequence of SEQ ID NO: 66, 31, 10, or 168, or an amino acid sequence having at least about 85%, 90%, 95%, or 99% sequence identity to SEQ ID NO: 66, 31, 10, or 168.

In some embodiments, the anti-CD73 antibody molecule comprises a light chain variable region comprising the amino acid sequence of SEQ ID NO: 21, or an amino acid sequence having at least about 85%, 90%, 95%, or 99% sequence identity to SEQ ID NO: 21.

In some embodiments, the anti-CD73 antibody molecule comprises a heavy chain comprising the amino acid sequence of SEQ ID NO: 68, 33, 12, 115, 113, or 112, or an amino acid sequence having at least about 85%, 90%, 95%, or 99% sequence identity to SEQ ID NO: 68, 33, 12, 115, 113, or 112.

In some embodiments, the anti-CD73 antibody molecule comprises a light chain comprising the amino acid sequence of SEQ ID NO: 23, or an amino acid sequence having at least about 85%, 90%, 95%, or 99% sequence identity to SEQ ID NO: 23.

In some embodiments, the anti-CD73 antibody molecule comprises:

(i) a heavy chain variable region comprising the amino acid sequence of SEQ ID NO: 66 (or a sequence having at least about 85%, 90%, 95%, or 99% sequence identity thereto) and a light chain variable region comprising the amino acid sequence of SEQ ID NO: 21 (or a sequence having at least about 85%, 90%, 95%, or 99% sequence identity thereto);

(ii) a heavy chain variable region comprising the amino acid sequence of SEQ ID NO: 31 (or a sequence having at least about 85%, 90%, 95%, or 99% sequence identity thereto) and a light chain variable region comprising the amino acid sequence of SEQ ID NO: 21 (or a sequence having at least about 85%, 90%, 95%, or 99% sequence identity thereto);

(iii) a heavy chain variable region comprising the amino acid sequence of SEQ ID NO: 10 (or a sequence having at least about 85%, 90%, 95%, or 99% sequence identity thereto) and a light chain variable region comprising the amino acid sequence of SEQ ID NO: 21 (or a sequence having at least about 85%, 90%, 95%, or 99% sequence identity thereto); or

(iv) a heavy chain variable region comprising the amino acid sequence of SEQ ID NO: 168 (or a sequence having at least about 85%, 90%, 95%, or 99% sequence identity thereto) and a light chain variable region comprising the amino acid sequence of SEQ ID NO: 21 (or a sequence having at least about 85%, 90%, 95%, or 99% sequence identity thereto).

In some embodiments, the anti-CD73 antibody molecule comprises:

(i) a heavy chain comprising the amino acid sequence of SEQ ID NO: 68 (or a sequence having at least about 85%, 90%, 95%, or 99% sequence identity thereto) and a light chain comprising the amino acid sequence of SEQ ID NO: 23 (or a sequence having at least about 85%, 90%, 95%, or 99% sequence identity thereto);

(ii) a heavy chain comprising the amino acid sequence of SEQ ID NO: 115 (or a sequence having at least about 85%, 90%, 95%, or 99% sequence identity thereto) and a light chain comprising the amino acid sequence of SEQ ID NO: 23 (or a sequence having at least about 85%, 90%, 95%, or 99% sequence identity thereto);

(iii) a heavy chain comprising the amino acid sequence of SEQ ID NO: 33 (or a sequence having at least about 85%, 90%, 95%, or 99% sequence identity thereto) and a light chain comprising the amino acid sequence of SEQ ID NO: 23 (or a sequence having at least about 85%, 90%, 95%, or 99% sequence identity thereto);

(iv) a heavy chain comprising the amino acid sequence of SEQ ID NO: 113 (or a sequence having at least about 85%, 90%, 95%, or 99% sequence identity thereto) and a light chain comprising the amino acid sequence of SEQ ID NO: 23 (or a sequence having at least about 85%, 90%, 95%, or 99% sequence identity thereto);

(v) a heavy chain comprising the amino acid sequence of SEQ ID NO: 12 (or a sequence having at least about 85%, 90%, 95%, or 99% sequence identity thereto) and a light chain comprising the amino acid sequence of SEQ ID NO: 23 (or a sequence having at least about 85%, 90%, 95%, or 99% sequence identity thereto); or

(vi) a heavy chain comprising the amino acid sequence of SEQ ID NO: 112 (or a sequence having at least about 85%, 90%, 95%, or 99% sequence identity thereto) and a light chain comprising the amino acid sequence of SEQ ID NO: 23 (or a sequence having at least about 85%, 90%, 95%, or 99% sequence identity thereto).

In certain embodiments, the anti-CD73 antibody molecule comprises a heavy chain variable region having an amino acid sequence derived from a human VH3-23 germline sequence. In certain embodiments, the anti-CD73 antibody molecule comprises a light chain variable region having an amino acid sequence derived from a human VK3-15 germline sequence. In certain embodiments, the anti-CD73 antibody molecule comprises a heavy chain variable region having an amino acid sequence derived from a human VH3-23 germline sequence, and a light chain variable region having an amino acid sequence derived from a human VK3-15 germline sequence.

In certain embodiments, the anti-CD73 antibody molecule comprises a heavy chain variable region having an amino acid sequence derived from a human VH4-59 germline sequence. In certain embodiments, the anti-CD73 antibody molecule comprises a light chain variable region having an amino acid sequence derived from a human VK1-12 germline sequence. In certain embodiments, the anti-CD73 antibody molecule comprises a heavy chain variable region having an amino acid sequence derived from a human VH4-59 germline sequence, and a light chain variable region having an amino acid sequence derived from a human VK1-12 germline sequence.

In certain embodiments, the anti-CD73 antibody molecule is a monoclonal antibody or an antibody with single specificity. In certain embodiments, the anti-CD73 antibody molecule is a bispecific or multispecific antibody. The heavy and light chains of the anti-CD73 antibody molecule can be full-length (e.g., an antibody can include at least one or at least two complete heavy chains, and at least one or at least two complete light chains) or can include an antigen-binding fragment (e.g., a Fab, F(ab′)2, Fv, a single chain Fv fragment, a single domain antibody, a diabody (dAb), a bivalent or bispecific antibody or fragment thereof, a single domain variant thereof, or a camelid antibody).

In certain embodiments, the anti-CD73 antibody molecule has a heavy chain constant region (Fc) chosen from, e.g., the heavy chain constant regions of IgG1, IgG2, IgG3, IgG4, IgM, IgA1, IgA2, IgD, and IgE; particularly, chosen from, e.g., the heavy chain constant regions of IgG1, IgG2, IgG3, and IgG4, more particularly, the heavy chain constant region of IgG4. In some embodiments, the heavy chain constant region is human IgG4. In some embodiments, the anti-CD73 antibody molecule has a light chain constant region chosen from, e.g., the light chain constant regions of kappa or lambda. In some embodiments, the light chain constant region is kappa (e.g., human kappa). In some embodiments, the constant region is altered, e.g., mutated, to modify the properties of the anti-CD73 antibody molecule (e.g., to increase or decrease one or more of: Fc receptor binding, antibody glycosylation, the number of cysteine residues, effector cell function, or complement function). In some embodiments, the heavy chain constant region of an IgG4, e.g., a human IgG4, is mutated at position 228 according to Eu numbering (e.g., S to P), e.g., as shown in Table 3. In certain embodiments, the anti-CD73 antibody molecules comprises a human IgG4 mutated at position 228 according to Eu numbering (e.g., S to P), e.g., as shown in Table 3; and a kappa light chain constant region, e.g., as shown in Table 3. In some embodiments, the heavy chain constant region of an IgG4, e.g., a human IgG4, is mutated at position 228 (e.g., S to P) and position 235 (e.g., L to E) according to Eu numbering, e.g., as shown in Table 3. In certain embodiments, the anti-CD73 antibody molecules comprises a human IgG4 mutated at position 228 (e.g., S to P) and position 235 (e.g., L to E) according to Eu numbering, e.g., as shown in Table 3; and a kappa light chain constant region, e.g., as shown in Table 3. In still another embodiment, the heavy chain constant region of an IgG1, e.g., a human IgG1, is mutated at one or more of position 297 (e.g., N to A), position 265 (e.g., D to A), position 329 (e.g., P to A), position 234 (e.g., L to A), or position 235 (e.g., L to A), all according to Eu numbering, e.g., as shown in Table 3. In certain embodiments, the anti-CD73 antibody molecules comprises a human IgG1 mutated at one or more of the aforesaid positions, e.g., as shown in Table 3; and a kappa light chain constant region, e.g., as shown in Table 3.

In some embodiments, the anti-CD73 antibody molecule comprises a heavy chain constant region selected from IgG1, IgG2, IgG3, and IgG4, and a light chain constant region chosen from the light chain constant regions of kappa or lambda. In some embodiments, the anti-CD73 antibody molecule comprises a heavy chain constant region comprising an amino acid sequence selected from the group consisting of SEQ ID NOs: 92-103, 119, and 120, and a light chain constant region comprising the amino acid sequence of SEQ ID NO: 104.

In one aspect, the invention features an antibody molecule that competes with a monoclonal antibody, e.g., an antibody molecule described herein, for binding to human CD73. The invention also features an antibody molecule that binds to the same (or substantially the same) or an overlapping (or substantially overlapping) epitope as a monoclonal antibody, e.g., an antibody molecule described herein, to human CD73.

In one embodiment, the monoclonal antibody comprises a heavy chain variable region (VH) comprising a heavy chain complementarity determining region 3 (VHCDR3) amino acid sequence of GGLYGSGSYLSDFDL (SEQ ID NO: 37).

In certain embodiments, the monoclonal antibody comprises:

(i) a heavy chain variable region (VH) comprising a heavy chain complementarity determining region 1 (VHCDR1) amino acid sequence of X₁X₂AMS (SEQ ID NO: 88), wherein X₁ is R, Y, or S, and X₂ is Y or N; a VHCDR2 amino acid sequence of X₁IX₂GX₃GX₄X₅TYYADSVKG (SEQ ID NO: 89), wherein X₁ is A or S, X₂ is S or T, X₃ is S or T, X₄ is M, G, or S, and X₅ is N, S, L, or Y; and a VHCDR3 amino acid sequence of GGLYGSGSYLSDFDL (SEQ ID NO: 37); and/or

(ii) a light chain variable region (VL) comprising a light chain complementarity determining region 1 (VLCDR1) amino acid sequence of RASQSVGSNLA (SEQ ID NO: 48); a VLCDR2 amino acid sequence of GASTRAT (SEQ ID NO: 49); and a VLCDR3 amino acid sequence of QQHNAFPYT (SEQ ID NO: 50).

In certain embodiments, the monoclonal antibody comprises:

(i) a VH comprising a VHCDR1 amino acid sequence of SEQ ID NO: 38, a VHCDR2 amino acid sequence of SEQ ID NO: 36, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(ii) a VH comprising a VHCDR1 amino acid sequence of SEQ ID NO: 72, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(iii) a VH comprising a VHCDR1 amino acid sequence of SEQ ID NO: 38, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(iv) a VH comprising a VHCDR1 amino acid sequence of SEQ ID NO: 137, a VHCDR2 amino acid sequence of SEQ ID NO: 136, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(v) a VH comprising a VHCDR1 amino acid sequence of SEQ ID NO: 137, a VHCDR2 amino acid sequence of SEQ ID NO: 146, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; or

(vi) a VH comprising a VHCDR1 amino acid sequence of SEQ ID NO: 137, a VHCDR2 amino acid sequence of SEQ ID NO: 154, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50.

In one embodiment, the monoclonal antibody comprises a heavy chain variable region (VH) comprising a heavy chain complementarity determining region 3 (VHCDR3) amino acid sequence of ESQESPYNNWFDP (SEQ ID NO: 3).

In certain embodiments, the monoclonal antibody comprises:

(i) a heavy chain variable region (VH) comprising a heavy chain complementarity determining region 1 (VHCDR1) amino acid sequence of X₁X₂YWS (SEQ ID NO: 90), wherein X₁ is R, G, or S, and X₂ is Y or R; a VHCDR2 amino acid sequence of YIYX₁X₂GSTX₃YNPSLKS (SEQ ID NO: 91), wherein X₁ is G or S, X₂ is R, S, or T, and X₃ is N or K; and a VHCDR3 amino acid sequence of ESQESPYNNWFDP (SEQ ID NO: 3); and/or

(ii) a light chain variable region (VL) comprising a light chain complementarity determining region 1 (VLCDR1) amino acid sequence of RASQGISSWLA (SEQ ID NO: 14); a VLCDR2 amino acid sequence of AASSLQS (SEQ ID NO: 15); and a VLCDR3 amino acid sequence of QQGNSFPRT (SEQ ID NO: 16).

In certain embodiments, the monoclonal antibody comprises:

(i) a VH comprising a VHCDR1 amino acid sequence of SEQ ID NO: 61, a VHCDR2 amino acid sequence of SEQ ID NO: 60, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(ii) a VH comprising a VHCDR1 amino acid sequence of SEQ ID NO: 4, a VHCDR2 amino acid sequence of SEQ ID NO: 26, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(iii) a VH comprising a VHCDR1 amino acid sequence of SEQ ID NO: 4, a VHCDR2 amino acid sequence of SEQ ID NO: 2, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16; or

(iv) a VH comprising a VHCDR1 amino acid sequence of SEQ ID NO: 163, a VHCDR2 amino acid sequence of SEQ ID NO: 162, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16.

In one aspect, the invention features a nucleic acid molecule that comprise one or both nucleotide sequences that encode heavy and light chain variable regions, CDRs, hypervariable loops, framework regions of the anti-CD73 antibody molecules, as described herein. In certain embodiments, the nucleotide sequence that encodes the anti-CD73 antibody molecule is codon optimized. For example, the invention features a first and second nucleic acid encoding heavy and light chain variable regions, respectively, of an anti-CD73 antibody molecule chosen from, e.g., any of 918, 350, 356, 358, 930, 373, 374, 376, 377, or 379, as summarized in Table 1, or a sequence substantially identical thereto. For example, the nucleic acid can comprise a nucleotide sequence as set forth in Table 1, or a sequence substantially identical thereto (e.g., a sequence having at least about 85%, 90%, 95%, or 99% sequence identity thereto, or which differs by no more than 3, 6, 15, 30, or 45 nucleotides from the sequences shown in Table 1).

In one aspect, nucleic acids comprising nucleotide sequences that encode heavy and light chain variable regions and CDRs of the anti-CD73 antibody molecules, as described herein, are disclosed. For example, the disclosure provides a first and second nucleic acid encoding heavy and light chain variable regions, respectively, of an anti-CD73 antibody molecule according to Table 1 or a sequence substantially identical thereto. For example, the nucleic acid can comprise a nucleotide sequence encoding an anti-CD73 antibody molecule according to Table 1, or a sequence substantially identical to that nucleotide sequence (e.g., a sequence having at least about 85%, 90%, 95%, or 99% sequence identity thereto, or which differs by no more than 3, 6, 15, 30, or 45 nucleotides from the aforementioned nucleotide sequence).

In certain embodiments, the nucleic acid can comprise a nucleotide sequence encoding at least one, two, or three CDRs, or hypervariable loops, from a heavy chain variable region having an amino acid sequence as set forth in Table 1, or a sequence substantially homologous thereto (e.g., a sequence having at least about 85%, 90%, 95%, or 99% sequence identity thereto, and/or having one, two, three or more substitutions, insertions or deletions, e.g., conserved substitutions).

In certain embodiments, the nucleic acid can comprise a nucleotide sequence encoding at least one, two, or three CDRs, or hypervariable loops, from a light chain variable region having an amino acid sequence as set forth in Table 1, or a sequence substantially homologous thereto (e.g., a sequence having at least about 85%, 90%, 95%, or 99% sequence identity thereto, and/or having one, two, three or more substitutions, insertions or deletions, e.g., conserved substitutions).

In certain embodiments, the nucleic acid can comprise a nucleotide sequence encoding at least one, two, three, four, five, or six CDRs, or hypervariable loops, from heavy and light chain variable regions having an amino acid sequence as set forth in Table 1, or a sequence substantially homologous thereto (e.g., a sequence having at least about 85%, 90%, 95%, or 99% sequence identity thereto, and/or having one, two, three or more substitutions, insertions or deletions, e.g., conserved substitutions).

In certain embodiments, the nucleic acid encodes a heavy chain variable region, wherein the nucleic acid comprises the nucleotide sequence of SEQ ID NO: 45, 78, 85, 143, 152, 160, 67, 32, 11, or 169, or a nucleotide sequence having at least about 85%, 90%, 95%, or 99% sequence identity to SEQ ID NO: 45, 78, 85, 143, 152, 160, 67, 32, 11, or 169.

In certain embodiments, the nucleic acid encodes a heavy chain, wherein the nucleic acid comprises the nucleotide sequence of SEQ ID NO: 47, 80, 87, 69, 34, or 13, or a nucleotide sequence having at least about 85%, 90%, 95%, or 99% sequence identity to SEQ ID NO: 47, 80, 87, 69, 34, or 13.

In certain embodiments, the nucleic acid encodes a light chain variable region, wherein the nucleic acid comprises the nucleotide sequence of SEQ ID NO: 56, 144, 22, or 170, or a nucleotide sequence having at least about 85%, 90%, 95%, or 99% sequence identity to SEQ ID NO: 56, 144, 22, or 170.

In certain embodiments, the nucleic acid encodes a light chain, wherein the nucleic acid comprises the nucleotide sequence of SEQ ID NO: 58 or 24, or a nucleotide sequence having at least about 85%, 90%, 95%, or 99% sequence identity to SEQ ID NO: 58 or 24.

In one aspect, this disclosure features host cells and vectors containing the nucleic acids described herein. The nucleic acids may be present in a single vector or separate vectors present in the same host cell or separate host cell. Also provided herein is a method of producing an anti-C73 antibody molecule, the method comprising culturing a host cell disclosed herein under conditions suitable for gene expression.

In one aspect, the present disclosure provides a method of providing an antibody molecule described herein. The method may include: providing a CD73 antigen (e.g., an antigen comprising at least a portion of a CD73 epitope, e.g., the N-terminal domain of a CD73 antigen); obtaining an antibody molecule that binds to the CD73 antigen; and evaluating if the antibody molecule binds to the CD73 antigen, or evaluating efficacy of the antibody molecule in modulating, e.g., stimulating or inhibiting, the activity of CD73. The method can further include administering the antibody molecule to a subject, e.g., a human or non-human animal

In one aspect, the disclosure provides, compositions, e.g., pharmaceutical compositions, which include a pharmaceutically acceptable carrier, excipient or stabilizer, and at least one of the anti-CD73 antibody molecules described herein. In one embodiment, the composition, e.g., the pharmaceutical composition, includes a combination of the antibody molecule and one or more agents, e.g., a therapeutic agent or other antibody molecule, as described herein. In some embodiments, the antibody molecule is conjugated to a label or a therapeutic agent. In some embodiments, the compositions, e.g., the pharmaceutical compositions, comprise a combination of the antibody molecule and a second agent, e.g., a therapeutic agent, or two or more of the aforesaid antibody molecules, as further described herein.

The anti-CD73 antibody molecules disclosed herein can inhibit, reduce or neutralize one or more activities of CD73, e.g., inhibiting or reducing the enzymatic activity of CD73; inhibiting or reducing the conversion of adenosine monophosphate (AMP) to adenosine; increasing proliferation of anti-CD3/anti-CD28 stimulated T cells, e.g., CD4+ T cells, in the presence of adenosine monophosphate (AMP); inhibiting proliferation of regulatory T cells; increasing effector T cell responses; and/or inhibiting migration, infiltration, or expansion of myeloid derived suppressor cells. Thus, such antibody molecules can be used to treat or prevent disorders where enhancing an immune response in a subject is desired.

In one aspect, disclosed herein is an antibody molecule that binds to human CD73, wherein:

(i) the antibody molecule binds to a human CD73 dimer, said dimer consisting of a first CD73 monomer and a second CD73 monomer (e.g., each monomer comprising the amino acid sequence of residues 27-547 of SEQ ID NO: 105, e.g., each monomer consists of the amino acid sequence of SEQ ID NO: 171), wherein when the antibody molecule comprises a first antigen binding domain and a second antigen binding domain, the first antigen binding domain binds to the first CD73 monomer and the second antigen binding domain binds to the second CD73 monomer, e.g., when tested using size exclusion chromatography; and

(ii) the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 368-387 of SEQ ID NO: 105 to a greater extent than at residues 158-172, residues 206-215, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature.

In one embodiment, antibody binding reduces the average hydrogen-deuterium exchange at residues 368-387 of SEQ ID NO: 105 to a greater extent than at residues 158-172 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105. In one embodiment, antibody binding reduces the average hydrogen-deuterium exchange at residues 368-387 of SEQ ID NO: 105 to a greater extent than at residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 206-215 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105, residues 206-215 of SEQ ID NO: 105, and residues 297-309 of SEQ ID NO: 105.

In one aspect, disclosed herein is an antibody molecule that binds to human CD73, wherein:

(i) the antibody molecule binds to a human CD73 dimer, said dimer consisting of a first CD73 monomer and a second CD73 monomer (e.g., each monomer comprising the amino acid sequence of residues 27-547 of SEQ ID NO: 105, e.g., each monomer consists of the amino acid sequence of SEQ ID NO: 171), wherein when the antibody molecule comprises a first antigen binding domain and a second antigen binding domain, the first antigen binding domain binds to the first CD73 monomer and the second antigen binding domain binds to the second CD73 monomer, e.g., when tested using size exclusion chromatography; and

(ii) the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 158-172 of SEQ ID NO: 105 to a greater extent than at residues 206-215 of SEQ ID NO: 105, residues 368-387 of SEQ ID NO: 105, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature.

In one embodiment, antibody binding reduces the average hydrogen-deuterium exchange at residues 158-172 of SEQ ID NO: 105 to a greater extent than at residues 206-215 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105. In one embodiment, antibody binding reduces the average hydrogen-deuterium exchange at residues 158-172 of SEQ ID NO: 105 to a greater extent than at residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 368-387 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105, residues 368-387 of SEQ ID NO: 105, and residues 297-309 of SEQ ID NO: 105.

In one aspect, disclosed herein is an antibody molecule that binds to human CD73, wherein:

(i) the antibody molecule binds to a human CD73 dimer, said dimer consisting of a first CD73 monomer and a second CD73 monomer (e.g., each monomer comprising the amino acid sequence of residues 27-547 of SEQ ID NO: 105, e.g., each monomer consists of the amino acid sequence of SEQ ID NO: 171), wherein when the antibody molecule comprises a first antigen binding domain and a second antigen binding domain, the first antigen binding domain binds to the first CD73 monomer and the second antigen binding domain binds to the second CD73 monomer, e.g., when tested using size exclusion chromatography; and

(ii) the antibody molecule reduces hydrogen-deuterium exchange at one or more regions of a protein comprising the amino acid sequence of residues 27-547 of SEQ ID NO: 105 (e.g., a protein consisting of the amino acid sequence of SEQ ID NO: 171) when bound thereto, wherein the one or more regions are selected from the group consisting of residues 158-172, residues 206-215, residues 368-387, and residues 87-104 of SEQ ID NO: 105, wherein the region having the greatest reduction in the average hydrogen-deuterium exchange among the one or more regions is not residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature.

In one aspect, disclosed herein is an antibody molecule that binds to human CD73, wherein:

(i) the antibody molecule binds to a human CD73 dimer, said dimer consisting of a first CD73 monomer and a second CD73 monomer (e.g., each monomer comprising the amino acid sequence of residues 27-547 of SEQ ID NO: 105, e.g., each monomer consists of the amino acid sequence of SEQ ID NO: 171), wherein when the antibody molecule comprises a first antigen binding domain and a second antigen binding domain, the first antigen binding domain binds to the first CD73 monomer and the second antigen binding domain binds to the second CD73 monomer, e.g., when tested using size exclusion chromatography; and

(ii) the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of more than, e.g., 0.02, 0.03, 0.04, 0.05, or 0.06 Da per residue at residues 368-387 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature.

In one aspect, disclosed herein is an antibody molecule that binds to human CD73, wherein:

(i) the antibody molecule binds to a human CD73 dimer, said dimer consisting of a first CD73 monomer and a second CD73 monomer (e.g., each monomer comprising the amino acid sequence of residues 27-547 of SEQ ID NO: 105, e.g., each monomer consists of the amino acid sequence of SEQ ID NO: 171), wherein when the antibody molecule comprises a first antigen binding domain and a second antigen binding domain, the first antigen binding domain binds to the first CD73 monomer and the second antigen binding domain binds to the second CD73 monomer, e.g., when tested using size exclusion chromatography; and

(ii) the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of less than, e.g., 0.05, 0.04, 0.03, or 0.02 Da per residue at residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature.

In one aspect, disclosed herein is an antibody molecule that binds to human CD73, wherein:

(i) the antibody molecule binds to a human CD73 dimer, said dimer consisting of a first CD73 monomer and a second CD73 monomer (e.g., each monomer comprising the amino acid sequence of residues 27-547 of SEQ ID NO: 105, e.g., each monomer consists of the amino acid sequence of SEQ ID NO: 171), wherein when the antibody molecule comprises a first antigen binding domain and a second antigen binding domain, the first antigen binding domain binds to the first CD73 monomer and the second antigen binding domain binds to the second CD73 monomer, e.g., when tested using size exclusion chromatography; and

(ii) the antibody molecule binds to at least one, two, three, or four residues within residues 158-172 of SEQ ID NO: 105, and/or at least one, two, three, four or five residues within residues 206-215 of SEQ ID NO: 105.

In one aspect, disclosed herein is an antibody molecule that binds to human CD73, wherein:

(i) the antibody molecule binds to a human CD73 dimer, said dimer consisting of a first CD73 monomer and a second CD73 monomer (e.g., each monomer comprising the amino acid sequence of residues 27-547 of SEQ ID NO: 105, e.g., each monomer consists of the amino acid sequence of SEQ ID NO: 171), wherein when the antibody molecule comprises a first antigen binding domain and a second antigen binding domain, the first antigen binding domain binds to the first CD73 monomer and the second antigen binding domain binds to the second CD73 monomer, e.g., when tested using size exclusion chromatography; and

(ii) the antibody molecule inhibits at least about 60%, 70%, 80%, or 90% of the enzymatic activity of membrane-bound human CD73, e.g., when the antibody molecule is tested as a bivalent antibody molecule using a modified Cell Titer Glo (CTG) assay, e.g., as described in Example 1.

In one aspect, disclosed herein is an antibody molecule that binds to human CD73, wherein:

(i) the antibody molecule binds to a human CD73 dimer, said dimer consisting of a first CD73 monomer and a second CD73 monomer (e.g., each monomer comprising the amino acid sequence of residues 27-547 of SEQ ID NO: 105, e.g., each monomer consists of the amino acid sequence of SEQ ID NO: 171), wherein when the antibody molecule comprises a first antigen binding domain and a second antigen binding domain, the first antigen binding domain binds to the first CD73 monomer and the second antigen binding domain binds to the second CD73 monomer, e.g., when tested using size exclusion chromatography; and

(ii) the antibody molecule comprises a heavy chain variable region (VH) comprising a heavy chain complementarity determining region 3 (VHCDR3) amino acid sequence of GGLYGSGSYLSDFDL (SEQ ID NO: 37).

In one aspect, disclosed herein is an antibody molecule that binds to human CD73, wherein:

(i) the antibody molecule binds to a human CD73 dimer, said dimer consisting of a first CD73 monomer and a second CD73 monomer (e.g., each monomer comprising the amino acid sequence of residues 27-547 of SEQ ID NO: 105, e.g., each monomer consists of the amino acid sequence of SEQ ID NO: 171), wherein when the antibody molecule comprises a first antigen binding domain and a second antigen binding domain, the first antigen binding domain binds to the first CD73 monomer and the second antigen binding domain binds to the second CD73 monomer, e.g., when tested using size exclusion chromatography; and

(ii) the antibody molecule comprises:

(a) a heavy chain variable region (VH) comprising a heavy chain complementarity determining region 1 (VHCDR1) amino acid sequence of X₁X₂AMS (SEQ ID NO: 88), wherein X₁ is R, Y, or S, and X₂ is Y or N; a VHCDR2 amino acid sequence of X₁IX₂GX₃GX₄X₅TYYADSVKG (SEQ ID NO: 89), wherein X₁ is A or S, X₂ is S or T, X₃ is S or T, X₄ is M, G, or S, and X₅ is N, S, L, or Y; and a VHCDR3 amino acid sequence of GGLYGSGSYLSDFDL (SEQ ID NO: 37); and

(b) a light chain variable region (VL) comprising a light chain complementarity determining region 1 (VLCDR1) amino acid sequence of RASQSVGSNLA (SEQ ID NO: 48); a VLCDR2 amino acid sequence of GASTRAT (SEQ ID NO: 49); and a VLCDR3 amino acid sequence of QQHNAFPYT (SEQ ID NO: 50).

In one aspect, disclosed herein is an antibody molecule that binds to human CD73, wherein:

(i) the antibody molecule binds to a human CD73 dimer, said dimer consisting of a first CD73 monomer and a second CD73 monomer (e.g., each monomer comprising the amino acid sequence of residues 27-547 of SEQ ID NO: 105, e.g., each monomer consists of the amino acid sequence of SEQ ID NO: 171), wherein when the antibody molecule comprises a first antigen binding domain and a second antigen binding domain, the first antigen binding domain binds to the first CD73 monomer and the second antigen binding domain binds to the second CD73 monomer, e.g., when tested using size exclusion chromatography; and

(ii) the antibody molecule comprises:

(a) a VH comprising a VHCDR1 amino acid sequence of SEQ ID NO: 38, a VHCDR2 amino acid sequence of SEQ ID NO: 36, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(b) a VH comprising a VHCDR1 amino acid sequence of SEQ ID NO: 72, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(c) a VH comprising a VHCDR1 amino acid sequence of SEQ ID NO: 38, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(d) a VH comprising a VHCDR1 amino acid sequence of SEQ ID NO: 137, a VHCDR2 amino acid sequence of SEQ ID NO: 136, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(e) a VH comprising a VHCDR1 amino acid sequence of SEQ ID NO: 137, a VHCDR2 amino acid sequence of SEQ ID NO: 146, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; or

(f) a VH comprising a VHCDR1 amino acid sequence of SEQ ID NO: 137, a VHCDR2 amino acid sequence of SEQ ID NO: 154, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50.

In one aspect, disclosed herein is an antibody molecule that binds to human CD73, wherein:

(i) the antibody molecule binds to a human CD73 dimer, said dimer consisting of a first CD73 monomer and a second CD73 monomer (e.g., each monomer comprising the amino acid sequence of residues 27-547 of SEQ ID NO: 105, e.g., each monomer consists of the amino acid sequence of SEQ ID NO: 171), wherein when the antibody molecule comprises a first antigen binding domain and a second antigen binding domain, the first antigen binding domain binds to the first CD73 monomer and the second antigen binding domain binds to the second CD73 monomer, e.g., when tested using size exclusion chromatography; and

(ii) the antibody molecule comprises a heavy chain variable region (VH) comprising a heavy chain complementarity determining region 3 (VHCDR3) amino acid sequence of ESQESPYNNWFDP (SEQ ID NO: 3).

In one aspect, disclosed herein is an antibody molecule that binds to human CD73, wherein:

(i) the antibody molecule binds to a human CD73 dimer, said dimer consisting of a first CD73 monomer and a second CD73 monomer (e.g., each monomer comprising the amino acid sequence of residues 27-547 of SEQ ID NO: 105, e.g., each monomer consists of the amino acid sequence of SEQ ID NO: 171), wherein when the antibody molecule comprises a first antigen binding domain and a second antigen binding domain, the first antigen binding domain binds to the first CD73 monomer and the second antigen binding domain binds to the second CD73 monomer, e.g., when tested using size exclusion chromatography; and

(ii) the antibody molecule comprises:

(a) a heavy chain variable region (VH) comprising a heavy chain complementarity determining region 1 (VHCDR1) amino acid sequence of X₁X₂YWS (SEQ ID NO: 90), wherein X₁ is R, G, or S, and X₂ is Y or R; a VHCDR2 amino acid sequence of YIYX₁X₂GSTX₃YNPSLKS (SEQ ID NO: 91), wherein X₁ is G or S, X₂ is R, S, or T, and X₃ is N or K; and a VHCDR3 amino acid sequence of ESQESPYNNWFDP (SEQ ID NO: 3); and

(b) a light chain variable region (VL) comprising a light chain complementarity determining region 1 (VLCDR1) amino acid sequence of RASQGISSWLA (SEQ ID NO: 14); a VLCDR2 amino acid sequence of AASSLQS (SEQ ID NO: 15); and a VLCDR3 amino acid sequence of QQGNSFPRT (SEQ ID NO: 16).

In one aspect, disclosed herein is an antibody molecule that binds to human CD73, wherein:

(i) the antibody molecule binds to a human CD73 dimer, said dimer consisting of a first CD73 monomer and a second CD73 monomer (e.g., each monomer comprising the amino acid sequence of residues 27-547 of SEQ ID NO: 105, e.g., each monomer consists of the amino acid sequence of SEQ ID NO: 171), wherein when the antibody molecule comprises a first antigen binding domain and a second antigen binding domain, the first antigen binding domain binds to the first CD73 monomer and the second antigen binding domain binds to the second CD73 monomer, e.g., when tested using size exclusion chromatography; and

(ii) the antibody molecule comprises:

(a) a VH comprising a VHCDR1 amino acid sequence of SEQ ID NO: 61, a VHCDR2 amino acid sequence of SEQ ID NO: 60, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(b) a VH comprising a VHCDR1 amino acid sequence of SEQ ID NO: 4, a VHCDR2 amino acid sequence of SEQ ID NO: 26, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(c) a VH comprising a VHCDR1 amino acid sequence of SEQ ID NO: 4, a VHCDR2 amino acid sequence of SEQ ID NO: 2, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16; or

(d) a VH comprising a VHCDR1 amino acid sequence of SEQ ID NO: 163, a VHCDR2 amino acid sequence of SEQ ID NO: 162, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16.

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at least 30%, 35%, 40%, 45%, 50%, 55%, 60%, 65%, 70%, 75%, or 80% of the antibody molecules in said composition bind to the CD73 dimer to form a complex, wherein each of said complex consists of one antibody molecule and one CD73 dimer, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 368-387 of SEQ ID NO: 105 to a greater extent than at residues 158-172, residues 206-215, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature.

In one embodiment, antibody binding reduces the average hydrogen-deuterium exchange at residues 368-387 of SEQ ID NO: 105 to a greater extent than at residues 158-172 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105. In one embodiment, antibody binding reduces the average hydrogen-deuterium exchange at residues 368-387 of SEQ ID NO: 105 to a greater extent than at residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 206-215 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105, residues 206-215 of SEQ ID NO: 105, and residues 297-309 of SEQ ID NO: 105.

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at least 30%, 35%, 40%, 45%, 50%, 55%, 60%, 65%, 70%, 75%, or 80% of the antibody molecules in said composition bind to the CD73 dimer to form a complex, wherein each of said complex consists of one antibody molecule and one CD73 dimer, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 158-172 of SEQ ID NO: 105 to a greater extent than at residues 206-215 of SEQ ID NO: 105, residues 368-387 of SEQ ID NO: 105, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature.

In one embodiment, antibody binding reduces the average hydrogen-deuterium exchange at residues 158-172 of SEQ ID NO: 105 to a greater extent than at residues 206-215 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105. In one embodiment, antibody binding reduces the average hydrogen-deuterium exchange at residues 158-172 of SEQ ID NO: 105 to a greater extent than at residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 368-387 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105, residues 368-387 of SEQ ID NO: 105, and residues 297-309 of SEQ ID NO: 105.

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at least 30%, 35%, 40%, 45%, 50%, 55%, 60%, 65%, 70%, 75%, or 80% of the antibody molecules in said composition bind to the CD73 dimer to form a complex, wherein each of said complex consists of one antibody molecule and one CD73 dimer, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule reduces hydrogen-deuterium exchange at one or more regions of a protein comprising the amino acid sequence of residues 27-547 of SEQ ID NO: 105 (e.g., a protein consisting of the amino acid sequence of SEQ ID NO: 171) when bound thereto, wherein the one or more regions are selected from the group consisting of residues 158-172, residues 206-215, residues 368-387, and residues 87-104 of SEQ ID NO: 105, wherein the region having the greatest reduction in the average hydrogen-deuterium exchange among the one or more regions is not residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature.

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at least 30%, 35%, 40%, 45%, 50%, 55%, 60%, 65%, 70%, 75%, or 80% of the antibody molecules in said composition bind to the CD73 dimer to form a complex, wherein each of said complex consists of one antibody molecule and one CD73 dimer, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of more than, e.g., 0.02, 0.03, 0.04, 0.05, or 0.06 Da per residue at residues 368-387 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature.

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at least 30%, 35%, 40%, 45%, 50%, 55%, 60%, 65%, 70%, 75%, or 80% of the antibody molecules in said composition bind to the CD73 dimer to form a complex, wherein each of said complex consists of one antibody molecule and one CD73 dimer, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of less than, e.g., 0.05, 0.04, 0.03, or 0.02 Da per residue at residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature.

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at least 30%, 35%, 40%, 45%, 50%, 55%, 60%, 65%, 70%, 75%, or 80% of the antibody molecules in said composition bind to the CD73 dimer to form a complex, wherein each of said complex consists of one antibody molecule and one CD73 dimer, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule binds to at least one, two, three, or four residues within residues 158-172 of SEQ ID NO: 105, and/or at least one, two, three, four or five residues within residues 206-215 of SEQ ID NO: 105.

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at least 30%, 35%, 40%, 45%, 50%, 55%, 60%, 65%, 70%, 75%, or 80% of the antibody molecules in said composition bind to the CD73 dimer to form a complex, wherein each of said complex consists of one antibody molecule and one CD73 dimer, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule inhibits at least about 60%, 70%, 80%, or 90% of the enzymatic activity of membrane-bound human CD73, e.g., when the antibody molecule is tested as a bivalent antibody molecule using a modified Cell Titer Glo (CTG) assay, e.g., as described in Example 1.

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at least 30%, 35%, 40%, 45%, 50%, 55%, 60%, 65%, 70%, 75%, or 80% of the antibody molecules in said composition bind to the CD73 dimer to form a complex, wherein each of said complex consists of one antibody molecule and one CD73 dimer, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule comprises a heavy chain variable region (VH) comprising a heavy chain complementarity determining region 3 (VHCDR3) amino acid sequence of GGLYGSGSYLSDFDL (SEQ ID NO: 37).

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) the antibody molecules in the plurality each comprises the same first antigen binding domain and the same second antigen binding domain, at least 30%, 35%, 40%, 45%, 50%, 55%, 60%, 65%, 70%, 75%, or 80% of the antibody molecules in said composition bind to the CD73 dimer to form a complex, wherein each of said complex consists of one antibody molecule and one CD73 dimer, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule comprises:

(a) a heavy chain variable region (VH) comprising a heavy chain complementarity determining region 1 (VHCDR1) amino acid sequence of X₁X₂AMS (SEQ ID NO: 88), wherein X₁ is R, Y, or S, and X₂ is Y or N; a VHCDR2 amino acid sequence of X₁IX₂GX₃GX₄X₅TYYADSVKG (SEQ ID NO: 89), wherein X₁ is A or S, X₂ is S or T, X₃ is S or T, X₄ is M, G, or S, and X₅ is N, S, L, or Y; and a VHCDR3 amino acid sequence of GGLYGSGSYLSDFDL (SEQ ID NO: 37); and

(b) a light chain variable region (VL) comprising a light chain complementarity determining region 1 (VLCDR1) amino acid sequence of RASQSVGSNLA (SEQ ID NO: 48); a VLCDR2 amino acid sequence of GASTRAT (SEQ ID NO: 49); and a VLCDR3 amino acid sequence of QQHNAFPYT (SEQ ID NO: 50).

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at least 30%, 35%, 40%, 45%, 50%, 55%, 60%, 65%, 70%, 75%, or 80% of the antibody molecules in said composition bind to the CD73 dimer to form a complex, wherein each of said complex consists of one antibody molecule and one CD73 dimer, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule comprises:

(a) a VH comprising a VHCDR1 amino acid sequence of SEQ ID NO: 38, a VHCDR2 amino acid sequence of SEQ ID NO: 36, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(b) a VH comprising a VHCDR1 amino acid sequence of SEQ ID NO: 72, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(c) a VH comprising a VHCDR1 amino acid sequence of SEQ ID NO: 38, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(d) a VH comprising a VHCDR1 amino acid sequence of SEQ ID NO: 137, a VHCDR2 amino acid sequence of SEQ ID NO: 136, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(e) a VH comprising a VHCDR1 amino acid sequence of SEQ ID NO: 137, a VHCDR2 amino acid sequence of SEQ ID NO: 146, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; or

(f) a VH comprising a VHCDR1 amino acid sequence of SEQ ID NO: 137, a VHCDR2 amino acid sequence of SEQ ID NO: 154, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50.

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at least 30%, 35%, 40%, 45%, 50%, 55%, 60%, 65%, 70%, 75%, or 80% of the antibody molecules in said composition bind to the CD73 dimer to form a complex, wherein each of said complex consists of one antibody molecule and one CD73 dimer, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule comprises a heavy chain variable region (VH) comprising a heavy chain complementarity determining region 3 (VHCDR3) amino acid sequence of ESQESPYNNWFDP (SEQ ID NO: 3).

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at least 30%, 35%, 40%, 45%, 50%, 55%, 60%, 65%, 70%, 75%, or 80% of the antibody molecules in said composition bind to the CD73 dimer to form a complex, wherein each of said complex consists of one antibody molecule and one CD73 dimer, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule comprises:

(a) a heavy chain variable region (VH) comprising a heavy chain complementarity determining region 1 (VHCDR1) amino acid sequence of X₁X₂YWS (SEQ ID NO: 90), wherein X₁ is R, G, or S, and X₂ is Y or R; a VHCDR2 amino acid sequence of YIYX₁X₂GSTX₃YNPSLKS (SEQ ID NO: 91), wherein X₁ is G or S, X₂ is R, S, or T, and X₃ is N or K; and a VHCDR3 amino acid sequence of ESQESPYNNWFDP (SEQ ID NO: 3); and

(b) a light chain variable region (VL) comprising a light chain complementarity determining region 1 (VLCDR1) amino acid sequence of RASQGISSWLA (SEQ ID NO: 14); a VLCDR2 amino acid sequence of AASSLQS (SEQ ID NO: 15); and a VLCDR3 amino acid sequence of QQGNSFPRT (SEQ ID NO: 16).

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at least 30%, 35%, 40%, 45%, 50%, 55%, 60%, 65%, 70%, 75%, or 80% of the antibody molecules in said composition bind to the CD73 dimer to form a complex, wherein each of said complex consists of one antibody molecule and one CD73 dimer, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule comprises:

(a) a VH comprising a VHCDR1 amino acid sequence of SEQ ID NO: 61, a VHCDR2 amino acid sequence of SEQ ID NO: 60, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(b) a VH comprising a VHCDR1 amino acid sequence of SEQ ID NO: 4, a VHCDR2 amino acid sequence of SEQ ID NO: 26, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(c) a VH comprising a VHCDR1 amino acid sequence of SEQ ID NO: 4, a VHCDR2 amino acid sequence of SEQ ID NO: 2, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16; or

(d) a VH comprising a VHCDR1 amino acid sequence of SEQ ID NO: 163, a VHCDR2 amino acid sequence of SEQ ID NO: 162, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16.

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at most 20%, 25%, 30%, 35%, 40%, 45%, 50%, 55%, 60%, 65%, or 70% of the antibody molecules in said composition bind to the CD73 dimer to form a complex, wherein each of said complex comprises two or more antibody molecules and two or more CD73 dimers, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 368-387 of SEQ ID NO: 105 to a greater extent than at residues 158-172, residues 206-215, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature.

In one embodiment, antibody binding reduces the average hydrogen-deuterium exchange at residues 368-387 of SEQ ID NO: 105 to a greater extent than at residues 158-172 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105. In one embodiment, antibody binding reduces the average hydrogen-deuterium exchange at residues 368-387 of SEQ ID NO: 105 to a greater extent than at residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 206-215 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105, residues 206-215 of SEQ ID NO: 105, and residues 297-309 of SEQ ID NO: 105.

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at most 20%, 25%, 30%, 35%, 40%, 45%, 50%, 55%, 60%, 65%, or 70% of the antibody molecules in said composition bind to the CD73 dimer to form a complex, wherein each of said complex comprises two or more antibody molecules and two or more CD73 dimers, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 158-172 of SEQ ID NO: 105 to a greater extent than at residues 206-215 of SEQ ID NO: 105, residues 368-387 of SEQ ID NO: 105, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature.

In one embodiment, antibody binding reduces the average hydrogen-deuterium exchange at residues 158-172 of SEQ ID NO: 105 to a greater extent than at residues 206-215 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105. In one embodiment, antibody binding reduces the average hydrogen-deuterium exchange at residues 158-172 of SEQ ID NO: 105 to a greater extent than at residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 368-387 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105, residues 368-387 of SEQ ID NO: 105, and residues 297-309 of SEQ ID NO: 105.

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at most 20%, 25%, 30%, 35%, 40%, 45%, 50%, 55%, 60%, 65%, or 70% of the antibody molecules in said composition bind to the CD73 dimer to form a complex, wherein each of said complex comprises two or more antibody molecules and two or more CD73 dimers, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule reduces hydrogen-deuterium exchange at one or more regions of a protein comprising the amino acid sequence of residues 27-547 of SEQ ID NO: 105 (e.g., a protein consisting of the amino acid sequence of SEQ ID NO: 171) when bound thereto, wherein the one or more regions are selected from the group consisting of residues 158-172, residues 206-215, residues 368-387, and residues 87-104 of SEQ ID NO: 105, wherein the region having the greatest reduction in the average hydrogen-deuterium exchange among the one or more regions is not residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature.

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at most 20%, 25%, 30%, 35%, 40%, 45%, 50%, 55%, 60%, 65%, or 70% of the antibody molecules in said composition bind to the CD73 dimer to form a complex, wherein each of said complex comprises two or more antibody molecules and two or more CD73 dimers, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of more than, e.g., 0.02, 0.03, 0.04, 0.05, or 0.06 Da per residue at residues 368-387 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature.

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at most 20%, 25%, 30%, 35%, 40%, 45%, 50%, 55%, 60%, 65%, or 70% of the antibody molecules in said composition bind to the CD73 dimer to form a complex, wherein each of said complex comprises two or more antibody molecules and two or more CD73 dimers, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of less than, e.g., 0.05, 0.04, 0.03, or 0.02 Da per residue at residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature.

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at most 20%, 25%, 30%, 35%, 40%, 45%, 50%, 55%, 60%, 65%, or 70% of the antibody molecules in said composition bind to the CD73 dimer to form a complex, wherein each of said complex comprises two or more antibody molecules and two or more CD73 dimers, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule binds to at least one, two, three, or four residues within residues 158-172 of SEQ ID NO: 105, and/or at least one, two, three, four or five residues within residues 206-215 of SEQ ID NO: 105.

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at most 20%, 25%, 30%, 35%, 40%, 45%, 50%, 55%, 60%, 65%, or 70% of the antibody molecules in said composition bind to the CD73 dimer to form a complex, wherein each of said complex comprises two or more antibody molecules and two or more CD73 dimers, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule inhibits at least about 60%, 70%, 80%, or 90% of the enzymatic activity of membrane-bound human CD73, e.g., when the antibody molecule is tested as a bivalent antibody molecule using a modified Cell Titer Glo (CTG) assay, e.g., as described in Example 1.

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at most 20%, 25%, 30%, 35%, 40%, 45%, 50%, 55%, 60%, 65%, or 70% of the antibody molecules in said composition bind to the CD73 dimer to form a complex, wherein each of said complex comprises two or more antibody molecules and two or more CD73 dimers, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule comprises a heavy chain variable region (VH) comprising a heavy chain complementarity determining region 3 (VHCDR3) amino acid sequence of GGLYGSGSYLSDFDL (SEQ ID NO: 37).

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at most 20%, 25%, 30%, 35%, 40%, 45%, 50%, 55%, 60%, 65%, or 70% of the antibody molecules in said composition bind to the CD73 dimer to form a complex, wherein each of said complex comprises two or more antibody molecules and two or more CD73 dimers, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule comprises:

(a) a heavy chain variable region (VH) comprising a heavy chain complementarity determining region 1 (VHCDR1) amino acid sequence of X₁X₂AMS (SEQ ID NO: 88), wherein X₁ is R, Y, or S, and X₂ is Y or N; a VHCDR2 amino acid sequence of X₁IX₂GX₃GX₄X₅TYYADSVKG (SEQ ID NO: 89), wherein X₁ is A or S, X₂ is S or T, X₃ is S or T, X₄ is M, G, or S, and X₅ is N, S, L, or Y; and a VHCDR3 amino acid sequence of GGLYGSGSYLSDFDL (SEQ ID NO: 37); and

(b) a light chain variable region (VL) comprising a light chain complementarity determining region 1 (VLCDR1) amino acid sequence of RASQSVGSNLA (SEQ ID NO: 48); a VLCDR2 amino acid sequence of GASTRAT (SEQ ID NO: 49); and a VLCDR3 amino acid sequence of QQHNAFPYT (SEQ ID NO: 50).

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at most 20%, 25%, 30%, 35%, 40%, 45%, 50%, 55%, 60%, 65%, or 70% of the antibody molecules in said composition bind to the CD73 dimer to form a complex, wherein each of said complex comprises two or more antibody molecules and two or more CD73 dimers, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule comprises:

(a) a VH comprising a VHCDR1 amino acid sequence of SEQ ID NO: 38, a VHCDR2 amino acid sequence of SEQ ID NO: 36, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(b) a VH comprising a VHCDR1 amino acid sequence of SEQ ID NO: 72, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(c) a VH comprising a VHCDR1 amino acid sequence of SEQ ID NO: 38, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(d) a VH comprising a VHCDR1 amino acid sequence of SEQ ID NO: 137, a VHCDR2 amino acid sequence of SEQ ID NO: 136, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(e) a VH comprising a VHCDR1 amino acid sequence of SEQ ID NO: 137, a VHCDR2 amino acid sequence of SEQ ID NO: 146, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; or
(f) a VH comprising a VHCDR1 amino acid sequence of SEQ ID NO: 137, a VHCDR2 amino acid sequence of SEQ ID NO: 154, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50.

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at most 20%, 25%, 30%, 35%, 40%, 45%, 50%, 55%, 60%, 65%, or 70% of the antibody molecules in said composition bind to the CD73 dimer to form a complex, wherein each of said complex comprises two or more antibody molecules and two or more CD73 dimers, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule comprises a heavy chain variable region (VH) comprising a heavy chain complementarity determining region 3 (VHCDR3) amino acid sequence of ESQESPYNNWFDP (SEQ ID NO: 3).

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at most 20%, 25%, 30%, 35%, 40%, 45%, 50%, 55%, 60%, 65%, or 70% of the antibody molecules in said composition bind to the CD73 dimer to form a complex, wherein each of said complex comprises two or more antibody molecules and two or more CD73 dimers, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule comprises:

(a) a heavy chain variable region (VH) comprising a heavy chain complementarity determining region 1 (VHCDR1) amino acid sequence of X₁X₂YWS (SEQ ID NO: 90), wherein X₁ is R, G, or S, and X₂ is Y or R; a VHCDR2 amino acid sequence of YIYX₁X₂GSTX₃YNPSLKS (SEQ ID NO: 91), wherein X₁ is G or S, X₂ is R, S, or T, and X₃ is N or K; and a VHCDR3 amino acid sequence of ESQESPYNNWFDP (SEQ ID NO: 3); and

(b) a light chain variable region (VL) comprising a light chain complementarity determining region 1 (VLCDR1) amino acid sequence of RASQGISSWLA (SEQ ID NO: 14); a VLCDR2 amino acid sequence of AASSLQS (SEQ ID NO: 15); and a VLCDR3 amino acid sequence of QQGNSFPRT (SEQ ID NO: 16).

In one aspect, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein:

(i) when the antibody molecules in the plurality each comprises the same first antigen binding domain and the same second antigen binding domain, at most 20%, 25%, 30%, 35%, 40%, 45%, 50%, 55%, 60%, 65%, or 70% of the antibody molecules in said composition bind to the CD73 dimer to form a complex, wherein each of said complex comprises two or more antibody molecules and two or more CD73 dimers, e.g., when measured using size exclusion chromatography and the percentage value is obtained by determining the amount of the antibody molecules in the complex relative to the total amount of the antibody molecules binding to CD73 (excluding unbound antibody molecules), e.g., as described in Example 2; and

(ii) the antibody molecule comprises:

(a) a VH comprising a VHCDR1 amino acid sequence of SEQ ID NO: 61, a VHCDR2 amino acid sequence of SEQ ID NO: 60, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(b) a VH comprising a VHCDR1 amino acid sequence of SEQ ID NO: 4, a VHCDR2 amino acid sequence of SEQ ID NO: 26, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(c) a VH comprising a VHCDR1 amino acid sequence of SEQ ID NO: 4, a VHCDR2 amino acid sequence of SEQ ID NO: 2, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16; or

(d) a VH comprising a VHCDR1 amino acid sequence of SEQ ID NO: 163, a VHCDR2 amino acid sequence of SEQ ID NO: 162, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16.

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 368-387 of SEQ ID NO: 105 to a greater extent than at residues 158-172, residues 206-215, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule inhibits at least about 60%, 70%, 80%, or 90% of the enzymatic activity of membrane-bound human CD73, e.g., when the antibody molecule is tested as a bivalent antibody molecule using a modified Cell Titer Glo (CTG) assay, e.g., as described in Example 1.

In one embodiment, antibody binding reduces the average hydrogen-deuterium exchange at residues 368-387 of SEQ ID NO: 105 to a greater extent than at residues 158-172 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105. In one embodiment, antibody binding reduces the average hydrogen-deuterium exchange at residues 368-387 of SEQ ID NO: 105 to a greater extent than at residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 206-215 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105, residues 206-215 of SEQ ID NO: 105, and residues 297-309 of SEQ ID NO: 105.

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 158-172 of SEQ ID NO: 105 to a greater extent than at residues 206-215 of SEQ ID NO: 105, residues 368-387 of SEQ ID NO: 105, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule inhibits at least about 60%, 70%, 80%, or 90% of the enzymatic activity of membrane-bound human CD73, e.g., when the antibody molecule is tested as a bivalent antibody molecule using a modified Cell Titer Glo (CTG) assay, e.g., as described in Example 1.

In one embodiment, antibody binding reduces the average hydrogen-deuterium exchange at residues 158-172 of SEQ ID NO: 105 to a greater extent than at residues 206-215 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105. In one embodiment, antibody binding reduces the average hydrogen-deuterium exchange at residues 158-172 of SEQ ID NO: 105 to a greater extent than at residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 368-387 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105, residues 368-387 of SEQ ID NO: 105, and residues 297-309 of SEQ ID NO: 105.

In one aspect, disclosed herein is an antibody molecule that binds to human CD73, wherein:

(i) the antibody molecule reduces hydrogen-deuterium exchange at one or more regions of a protein comprising the amino acid sequence of residues 27-547 of SEQ ID NO: 105 (e.g., a protein consisting of the amino acid sequence of SEQ ID NO: 171) when bound thereto, wherein the one or more regions are selected from the group consisting of residues 158-172, residues 206-215, residues 368-387, and residues 87-104 of SEQ ID NO: 105, wherein the region having the greatest reduction in the average hydrogen-deuterium exchange among the one or more regions is not residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule inhibits at least about 60%, 70%, 80%, or 90% of the enzymatic activity of membrane-bound human CD73, e.g., when the antibody molecule is tested as a bivalent antibody molecule using a modified Cell Titer Glo (CTG) assay, e.g., as described in Example 1.

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of more than, e.g., 0.02, 0.03, 0.04, 0.05, or 0.06 Da per residue at residues 368-387 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule inhibits at least about 60%, 70%, 80%, or 90% of the enzymatic activity of membrane-bound human CD73, e.g., when the antibody molecule is tested as a bivalent antibody molecule using a modified Cell Titer Glo (CTG) assay, e.g., as described in Example 1.

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of less than, e.g., 0.05, 0.04, 0.03, or 0.02 Da per residue at residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule inhibits at least about 60%, 70%, 80%, or 90% of the enzymatic activity of membrane-bound human CD73, e.g., when the antibody molecule is tested as a bivalent antibody molecule using a modified Cell Titer Glo (CTG) assay, e.g., as described in Example 1.

In one aspect, disclosed herein is an antibody molecule that binds to human CD73, wherein:

(i) the antibody molecule binds to at least one, two, three, or four residues within residues 158-172 of SEQ ID NO: 105, and/or at least one, two, three, four or five residues within residues 206-215 of SEQ ID NO: 105; and

(ii) the antibody molecule inhibits at least about 60%, 70%, 80%, or 90% of the enzymatic activity of membrane-bound human CD73, e.g., when the antibody molecule is tested as a bivalent antibody molecule using a modified Cell Titer Glo (CTG) assay, e.g., as described in Example 1.

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 368-387 of SEQ ID NO: 105 to a greater extent than at residues 158-172, residues 206-215, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and (ii) the antibody molecule comprises a heavy chain variable region (VH) comprising a heavy chain complementarity determining region 3 (VHCDR3) amino acid sequence of GGLYGSGSYLSDFDL (SEQ ID NO: 37).

In one embodiment, antibody binding reduces the average hydrogen-deuterium exchange at residues 368-387 of SEQ ID NO: 105 to a greater extent than at residues 158-172 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105. In one embodiment, antibody binding reduces the average hydrogen-deuterium exchange at residues 368-387 of SEQ ID NO: 105 to a greater extent than at residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 206-215 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105, residues 206-215 of SEQ ID NO: 105, and residues 297-309 of SEQ ID NO: 105.

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 158-172 of SEQ ID NO: 105 to a greater extent than at residues 206-215 of SEQ ID NO: 105, residues 368-387 of SEQ ID NO: 105, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises a heavy chain variable region (VH) comprising a heavy chain complementarity determining region 3 (VHCDR3) amino acid sequence of GGLYGSGSYLSDFDL (SEQ ID NO: 37).

In one embodiment, antibody binding reduces the average hydrogen-deuterium exchange at residues 158-172 of SEQ ID NO: 105 to a greater extent than at residues 206-215 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105. In one embodiment, antibody binding reduces the average hydrogen-deuterium exchange at residues 158-172 of SEQ ID NO: 105 to a greater extent than at residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 368-387 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105, residues 368-387 of SEQ ID NO: 105, and residues 297-309 of SEQ ID NO: 105.

In one aspect, disclosed herein is an antibody molecule that binds to human CD73, wherein:

(i) the antibody molecule reduces hydrogen-deuterium exchange at one or more regions of a protein comprising the amino acid sequence of residues 27-547 of SEQ ID NO: 105 (e.g., a protein consisting of the amino acid sequence of SEQ ID NO: 171) when bound thereto, wherein the one or more regions are selected from the group consisting of residues 158-172, residues 206-215, residues 368-387, and residues 87-104 of SEQ ID NO: 105, wherein the region having the greatest reduction in the average hydrogen-deuterium exchange among the one or more regions is not residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises a heavy chain variable region (VH) comprising a heavy chain complementarity determining region 3 (VHCDR3) amino acid sequence of GGLYGSGSYLSDFDL (SEQ ID NO: 37).

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of more than, e.g., 0.02, 0.03, 0.04, 0.05, or 0.06 Da per residue at residues 368-387 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises a heavy chain variable region (VH) comprising a heavy chain complementarity determining region 3 (VHCDR3) amino acid sequence of GGLYGSGSYLSDFDL (SEQ ID NO: 37).

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of less than, e.g., 0.05, 0.04, 0.03, or 0.02 Da per residue at residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises a heavy chain variable region (VH) comprising a heavy chain complementarity determining region 3 (VHCDR3) amino acid sequence of GGLYGSGSYLSDFDL (SEQ ID NO: 37).

In one aspect, disclosed herein is an antibody molecule that binds to human CD73, wherein:

(i) the antibody molecule binds to at least one, two, three, or four residues within residues 158-172 of SEQ ID NO: 105, and/or at least one, two, three, four or five residues within residues 206-215 of SEQ ID NO: 105; and

(ii) the antibody molecule comprises a heavy chain variable region (VH) comprising a heavy chain complementarity determining region 3 (VHCDR3) amino acid sequence of GGLYGSGSYLSDFDL (SEQ ID NO: 37).

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 368-387 of SEQ ID NO: 105 to a greater extent than at residues 158-172, residues 206-215, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and (ii) the antibody molecule comprises:

(a) a heavy chain variable region (VH) comprising a heavy chain complementarity determining region 1 (VHCDR1) amino acid sequence of X₁X₂AMS (SEQ ID NO: 88), wherein X₁ is R, Y, or S, and X₂ is Y or N; a VHCDR2 amino acid sequence of X₁IX₂GX₃GX₄X₅TYYADSVKG (SEQ ID NO: 89), wherein X₁ is A or S, X₂ is S or T, X₃ is S or T, X₄ is M, G, or S, and X₅ is N, S, L, or Y; and a VHCDR3 amino acid sequence of GGLYGSGSYLSDFDL (SEQ ID NO: 37); and

(b) a light chain variable region (VL) comprising a light chain complementarity determining region 1 (VLCDR1) amino acid sequence of RASQSVGSNLA (SEQ ID NO: 48); a VLCDR2 amino acid sequence of GASTRAT (SEQ ID NO: 49); and a VLCDR3 amino acid sequence of QQHNAFPYT (SEQ ID NO: 50).

In one embodiment, antibody binding reduces the average hydrogen-deuterium exchange at residues 368-387 of SEQ ID NO: 105 to a greater extent than at residues 158-172 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105. In one embodiment, antibody binding reduces the average hydrogen-deuterium exchange at residues 368-387 of SEQ ID NO: 105 to a greater extent than at residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 206-215 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105, residues 206-215 of SEQ ID NO: 105, and residues 297-309 of SEQ ID NO: 105.

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 158-172 of SEQ ID NO: 105 to a greater extent than at residues 206-215 of SEQ ID NO: 105, residues 368-387 of SEQ ID NO: 105, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises:

(a) a heavy chain variable region (VH) comprising a heavy chain complementarity determining region 1 (VHCDR1) amino acid sequence of X₁X₂AMS (SEQ ID NO: 88), wherein X₁ is R, Y, or S, and X₂ is Y or N; a VHCDR2 amino acid sequence of X₁IX₂GX₃GX₄X₅TYYADSVKG (SEQ ID NO: 89), wherein X₁ is A or S, X₂ is S or T, X₃ is S or T, X₄ is M, G, or S, and X₅ is N, S, L, or Y; and a VHCDR3 amino acid sequence of GGLYGSGSYLSDFDL (SEQ ID NO: 37); and

(b) a light chain variable region (VL) comprising a light chain complementarity determining region 1 (VLCDR1) amino acid sequence of RASQSVGSNLA (SEQ ID NO: 48); a VLCDR2 amino acid sequence of GASTRAT (SEQ ID NO: 49); and a VLCDR3 amino acid sequence of QQHNAFPYT (SEQ ID NO: 50).

In one embodiment, antibody binding reduces the average hydrogen-deuterium exchange at residues 158-172 of SEQ ID NO: 105 to a greater extent than at residues 206-215 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105. In one embodiment, antibody binding reduces the average hydrogen-deuterium exchange at residues 158-172 of SEQ ID NO: 105 to a greater extent than at residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 368-387 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105, residues 368-387 of SEQ ID NO: 105, and residues 297-309 of SEQ ID NO: 105.

In one aspect, disclosed herein is an antibody molecule that binds to human CD73, wherein:

(i) the antibody molecule reduces hydrogen-deuterium exchange at one or more regions of a protein comprising the amino acid sequence of residues 27-547 of SEQ ID NO: 105 (e.g., a protein consisting of the amino acid sequence of SEQ ID NO: 171) when bound thereto, wherein the one or more regions are selected from the group consisting of residues 158-172, residues 206-215, residues 368-387, and residues 87-104 of SEQ ID NO: 105, wherein the region having the greatest reduction in the average hydrogen-deuterium exchange among the one or more regions is not residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises:

(a) a heavy chain variable region (VH) comprising a heavy chain complementarity determining region 1 (VHCDR1) amino acid sequence of X₁X₂AMS (SEQ ID NO: 88), wherein X₁ is R, Y, or S, and X₂ is Y or N; a VHCDR2 amino acid sequence of X₁IX₂GX₃GX₄X₅TYYADSVKG (SEQ ID NO: 89), wherein X₁ is A or S, X₂ is S or T, X₃ is S or T, X₄ is M, G, or S, and X₅ is N, S, L, or Y; and a VHCDR3 amino acid sequence of GGLYGSGSYLSDFDL (SEQ ID NO: 37); and

(b) a light chain variable region (VL) comprising a light chain complementarity determining region 1 (VLCDR1) amino acid sequence of RASQSVGSNLA (SEQ ID NO: 48); a VLCDR2 amino acid sequence of GASTRAT (SEQ ID NO: 49); and a VLCDR3 amino acid sequence of QQHNAFPYT (SEQ ID NO: 50).

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of more than, e.g., 0.02, 0.03, 0.04, 0.05, or 0.06 Da per residue at residues 368-387 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises:

(a) a heavy chain variable region (VH) comprising a heavy chain complementarity determining region 1 (VHCDR1) amino acid sequence of X₁X₂AMS (SEQ ID NO: 88), wherein X₁ is R, Y, or S, and X₂ is Y or N; a VHCDR2 amino acid sequence of X₁IX₂GX₃GX₄X₅TYYADSVKG (SEQ ID NO: 89), wherein X₁ is A or S, X₂ is S or T, X₃ is S or T, X₄ is M, G, or S, and X₅ is N, S, L, or Y; and a VHCDR3 amino acid sequence of GGLYGSGSYLSDFDL (SEQ ID NO: 37); and

(b) a light chain variable region (VL) comprising a light chain complementarity determining region 1 (VLCDR1) amino acid sequence of RASQSVGSNLA (SEQ ID NO: 48); a VLCDR2 amino acid sequence of GASTRAT (SEQ ID NO: 49); and a VLCDR3 amino acid sequence of QQHNAFPYT (SEQ ID NO: 50).

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of less than, e.g., 0.05, 0.04, 0.03, or 0.02 Da per residue at residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises:

(a) a heavy chain variable region (VH) comprising a heavy chain complementarity determining region 1 (VHCDR1) amino acid sequence of X₁X₂AMS (SEQ ID NO: 88), wherein X₁ is R, Y, or S, and X₂ is Y or N; a VHCDR2 amino acid sequence of X₁IX₂GX₃GX₄X₅TYYADSVKG (SEQ ID NO: 89), wherein X₁ is A or S, X₂ is S or T, X₃ is S or T, X₄ is M, G, or S, and X₅ is N, S, L, or Y; and a VHCDR3 amino acid sequence of GGLYGSGSYLSDFDL (SEQ ID NO: 37); and

(b) a light chain variable region (VL) comprising a light chain complementarity determining region 1 (VLCDR1) amino acid sequence of RASQSVGSNLA (SEQ ID NO: 48); a VLCDR2 amino acid sequence of GASTRAT (SEQ ID NO: 49); and a VLCDR3 amino acid sequence of QQHNAFPYT (SEQ ID NO: 50).

In one aspect, disclosed herein is an antibody molecule that binds to human CD73, wherein:

(i) the antibody molecule binds to at least one, two, three, or four residues within residues 158-172 of SEQ ID NO: 105, and/or at least one, two, three, four or five residues within residues 206-215 of SEQ ID NO: 105; and

(ii) the antibody molecule comprises:

(a) a heavy chain variable region (VH) comprising a heavy chain complementarity determining region 1 (VHCDR1) amino acid sequence of X₁X₂AMS (SEQ ID NO: 88), wherein X₁ is R, Y, or S, and X₂ is Y or N; a VHCDR2 amino acid sequence of X₁IX₂GX₃GX₄X₅TYYADSVKG (SEQ ID NO: 89), wherein X₁ is A or S, X₂ is S or T, X₃ is S or T, X₄ is M, G, or S, and X₅ is N, S, L, or Y; and a VHCDR3 amino acid sequence of GGLYGSGSYLSDFDL (SEQ ID NO: 37); and

(b) a light chain variable region (VL) comprising a light chain complementarity determining region 1 (VLCDR1) amino acid sequence of RASQSVGSNLA (SEQ ID NO: 48); a VLCDR2 amino acid sequence of GASTRAT (SEQ ID NO: 49); and a VLCDR3 amino acid sequence of QQHNAFPYT (SEQ ID NO: 50).

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 368-387 of SEQ ID NO: 105 to a greater extent than at residues 158-172, residues 206-215, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises:

(a) a VH comprising a VHCDR1 amino acid sequence of SEQ ID NO: 38, a VHCDR2 amino acid sequence of SEQ ID NO: 36, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(b) a VH comprising a VHCDR1 amino acid sequence of SEQ ID NO: 72, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(c) a VH comprising a VHCDR1 amino acid sequence of SEQ ID NO: 38, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(d) a VH comprising a VHCDR1 amino acid sequence of SEQ ID NO: 137, a VHCDR2 amino acid sequence of SEQ ID NO: 136, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(e) a VH comprising a VHCDR1 amino acid sequence of SEQ ID NO: 137, a VHCDR2 amino acid sequence of SEQ ID NO: 146, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; or

(f) a VH comprising a VHCDR1 amino acid sequence of SEQ ID NO: 137, a VHCDR2 amino acid sequence of SEQ ID NO: 154, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50.

In one embodiment, antibody binding reduces the average hydrogen-deuterium exchange at residues 368-387 of SEQ ID NO: 105 to a greater extent than at residues 158-172 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105. In one embodiment, antibody binding reduces the average hydrogen-deuterium exchange at residues 368-387 of SEQ ID NO: 105 to a greater extent than at residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 206-215 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105, residues 206-215 of SEQ ID NO: 105, and residues 297-309 of SEQ ID NO: 105.

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 158-172 of SEQ ID NO: 105 to a greater extent than at residues 206-215 of SEQ ID NO: 105, residues 368-387 of SEQ ID NO: 105, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises:

(a) a VH comprising a VHCDR1 amino acid sequence of SEQ ID NO: 38, a VHCDR2 amino acid sequence of SEQ ID NO: 36, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(b) a VH comprising a VHCDR1 amino acid sequence of SEQ ID NO: 72, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(c) a VH comprising a VHCDR1 amino acid sequence of SEQ ID NO: 38, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(d) a VH comprising a VHCDR1 amino acid sequence of SEQ ID NO: 137, a VHCDR2 amino acid sequence of SEQ ID NO: 136, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(e) a VH comprising a VHCDR1 amino acid sequence of SEQ ID NO: 137, a VHCDR2 amino acid sequence of SEQ ID NO: 146, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; or

(f) a VH comprising a VHCDR1 amino acid sequence of SEQ ID NO: 137, a VHCDR2 amino acid sequence of SEQ ID NO: 154, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50.

In one embodiment, antibody binding reduces the average hydrogen-deuterium exchange at residues 158-172 of SEQ ID NO: 105 to a greater extent than at residues 206-215 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105. In one embodiment, antibody binding reduces the average hydrogen-deuterium exchange at residues 158-172 of SEQ ID NO: 105 to a greater extent than at residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 368-387 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105, residues 368-387 of SEQ ID NO: 105, and residues 297-309 of SEQ ID NO: 105.

In one aspect, disclosed herein is an antibody molecule that binds to human CD73, wherein:

(i) the antibody molecule reduces hydrogen-deuterium exchange at one or more regions of a protein comprising the amino acid sequence of residues 27-547 of SEQ ID NO: 105 (e.g., a protein consisting of the amino acid sequence of SEQ ID NO: 171) when bound thereto, wherein the one or more regions are selected from the group consisting of residues 158-172, residues 206-215, residues 368-387, and residues 87-104 of SEQ ID NO: 105, wherein the region having the greatest reduction in the average hydrogen-deuterium exchange among the one or more regions is not residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises:

(a) a VH comprising a VHCDR1 amino acid sequence of SEQ ID NO: 38, a VHCDR2 amino acid sequence of SEQ ID NO: 36, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(b) a VH comprising a VHCDR1 amino acid sequence of SEQ ID NO: 72, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(c) a VH comprising a VHCDR1 amino acid sequence of SEQ ID NO: 38, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(d) a VH comprising a VHCDR1 amino acid sequence of SEQ ID NO: 137, a VHCDR2 amino acid sequence of SEQ ID NO: 136, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(e) a VH comprising a VHCDR1 amino acid sequence of SEQ ID NO: 137, a VHCDR2 amino acid sequence of SEQ ID NO: 146, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; or

(f) a VH comprising a VHCDR1 amino acid sequence of SEQ ID NO: 137, a VHCDR2 amino acid sequence of SEQ ID NO: 154, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50.

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of more than, e.g., 0.02, 0.03, 0.04, 0.05, or 0.06 Da per residue at residues 368-387 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises:

(a) a VH comprising a VHCDR1 amino acid sequence of SEQ ID NO: 38, a VHCDR2 amino acid sequence of SEQ ID NO: 36, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(b) a VH comprising a VHCDR1 amino acid sequence of SEQ ID NO: 72, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(c) a VH comprising a VHCDR1 amino acid sequence of SEQ ID NO: 38, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(d) a VH comprising a VHCDR1 amino acid sequence of SEQ ID NO: 137, a VHCDR2 amino acid sequence of SEQ ID NO: 136, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(e) a VH comprising a VHCDR1 amino acid sequence of SEQ ID NO: 137, a VHCDR2 amino acid sequence of SEQ ID NO: 146, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; or

(f) a VH comprising a VHCDR1 amino acid sequence of SEQ ID NO: 137, a VHCDR2 amino acid sequence of SEQ ID NO: 154, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50.

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of less than, e.g., 0.05, 0.04, 0.03, or 0.02 Da per residue at residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises a heavy chain variable region (VH) comprising a heavy chain complementarity determining region 3 (VHCDR3) amino acid sequence of GGLYGSGSYLSDFDL (SEQ ID NO: 37).

In one aspect, disclosed herein is an antibody molecule that binds to human CD73, wherein:

(i) the antibody molecule binds to at least one, two, three, or four residues within residues 158-172 of SEQ ID NO: 105, and/or at least one, two, three, four or five residues within residues 206-215 of SEQ ID NO: 105; and

(ii) the antibody molecule comprises:

(a) a VH comprising a VHCDR1 amino acid sequence of SEQ ID NO: 38, a VHCDR2 amino acid sequence of SEQ ID NO: 36, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(b) a VH comprising a VHCDR1 amino acid sequence of SEQ ID NO: 72, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(c) a VH comprising a VHCDR1 amino acid sequence of SEQ ID NO: 38, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(d) a VH comprising a VHCDR1 amino acid sequence of SEQ ID NO: 137, a VHCDR2 amino acid sequence of SEQ ID NO: 136, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(e) a VH comprising a VHCDR1 amino acid sequence of SEQ ID NO: 137, a VHCDR2 amino acid sequence of SEQ ID NO: 146, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; or

(f) a VH comprising a VHCDR1 amino acid sequence of SEQ ID NO: 137, a VHCDR2 amino acid sequence of SEQ ID NO: 154, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50.

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 368-387 of SEQ ID NO: 105 to a greater extent than at residues 158-172, residues 206-215, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises a heavy chain variable region (VH) comprising a heavy chain complementarity determining region 3 (VHCDR3) amino acid sequence of ESQESPYNNWFDP (SEQ ID NO: 3).

In one embodiment, antibody binding reduces the average hydrogen-deuterium exchange at residues 368-387 of SEQ ID NO: 105 to a greater extent than at residues 158-172 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105. In one embodiment, antibody binding reduces the average hydrogen-deuterium exchange at residues 368-387 of SEQ ID NO: 105 to a greater extent than at residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 206-215 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105, residues 206-215 of SEQ ID NO: 105, and residues 297-309 of SEQ ID NO: 105.

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 158-172 of SEQ ID NO: 105 to a greater extent than at residues 206-215 of SEQ ID NO: 105, residues 368-387 of SEQ ID NO: 105, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises a heavy chain variable region (VH) comprising a heavy chain complementarity determining region 3 (VHCDR3) amino acid sequence of ESQESPYNNWFDP (SEQ ID NO: 3).

In one embodiment, antibody binding reduces the average hydrogen-deuterium exchange at residues 158-172 of SEQ ID NO: 105 to a greater extent than at residues 206-215 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105. In one embodiment, antibody binding reduces the average hydrogen-deuterium exchange at residues 158-172 of SEQ ID NO: 105 to a greater extent than at residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 368-387 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105, residues 368-387 of SEQ ID NO: 105, and residues 297-309 of SEQ ID NO: 105.

In one aspect, disclosed herein is an antibody molecule that binds to human CD73, wherein:

(i) the antibody molecule reduces hydrogen-deuterium exchange at one or more regions of a protein comprising the amino acid sequence of residues 27-547 of SEQ ID NO: 105 (e.g., a protein consisting of the amino acid sequence of SEQ ID NO: 171) when bound thereto, wherein the one or more regions are selected from the group consisting of residues 158-172, residues 206-215, residues 368-387, and residues 87-104 of SEQ ID NO: 105, wherein the region having the greatest reduction in the average hydrogen-deuterium exchange among the one or more regions is not residues 206-215 of SEQ ID NO:

105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises a heavy chain variable region (VH) comprising a heavy chain complementarity determining region 3 (VHCDR3) amino acid sequence of ESQESPYNNWFDP (SEQ ID NO: 3).

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of more than, e.g., 0.02, 0.03, 0.04, 0.05, or 0.06 Da per residue at residues 368-387 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises a heavy chain variable region (VH) comprising a heavy chain complementarity determining region 3 (VHCDR3) amino acid sequence of ESQESPYNNWFDP (SEQ ID NO: 3).

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of less than, e.g., 0.05, 0.04, 0.03, or 0.02 Da per residue at residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises a heavy chain variable region (VH) comprising a heavy chain complementarity determining region 3 (VHCDR3) amino acid sequence of ESQESPYNNWFDP (SEQ ID NO: 3).

In one aspect, disclosed herein is an antibody molecule that binds to human CD73, wherein:

(i) the antibody molecule binds to at least one, two, three, or four residues within residues 158-172 of SEQ ID NO: 105, and/or at least one, two, three, four or five residues within residues 206-215 of SEQ ID NO: 105; and

(ii) the antibody molecule comprises a heavy chain variable region (VH) comprising a heavy chain complementarity determining region 3 (VHCDR3) amino acid sequence of ESQESPYNNWFDP (SEQ ID NO: 3).

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 368-387 of SEQ ID NO: 105 to a greater extent than at residues 158-172, residues 206-215, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and (ii) the antibody molecule comprises:

(a) a heavy chain variable region (VH) comprising a heavy chain complementarity determining region 1 (VHCDR1) amino acid sequence of X₁X₂YWS (SEQ ID NO: 90), wherein X₁ is R, G, or S, and X₂ is Y or R; a VHCDR2 amino acid sequence of YIYX₁X₂GSTX₃YNPSLKS (SEQ ID NO: 91), wherein X₁ is G or S, X₂ is R, S, or T, and X₃ is N or K; and a VHCDR3 amino acid sequence of ESQESPYNNWFDP (SEQ ID NO: 3); and

(b) a light chain variable region (VL) comprising a light chain complementarity determining region 1 (VLCDR1) amino acid sequence of RASQGISSWLA (SEQ ID NO: 14); a VLCDR2 amino acid sequence of AASSLQS (SEQ ID NO: 15); and a VLCDR3 amino acid sequence of QQGNSFPRT (SEQ ID NO: 16).

In one embodiment, antibody binding reduces the average hydrogen-deuterium exchange at residues 368-387 of SEQ ID NO: 105 to a greater extent than at residues 158-172 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105. In one embodiment, antibody binding reduces the average hydrogen-deuterium exchange at residues 368-387 of SEQ ID NO: 105 to a greater extent than at residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 206-215 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105, residues 206-215 of SEQ ID NO: 105, and residues 297-309 of SEQ ID NO: 105.

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 158-172 of SEQ ID NO: 105 to a greater extent than at residues 206-215 of SEQ ID NO: 105, residues 368-387 of SEQ ID NO: 105, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises:

(a) a heavy chain variable region (VH) comprising a heavy chain complementarity determining region 1 (VHCDR1) amino acid sequence of X₁X₂YWS (SEQ ID NO: 90), wherein X₁ is R, G, or S, and X₂ is Y or R; a VHCDR2 amino acid sequence of YIYX₁X₂GSTX₃YNPSLKS (SEQ ID NO: 91), wherein X₁ is G or S, X₂ is R, S, or T, and X₃ is N or K; and a VHCDR3 amino acid sequence of ESQESPYNNWFDP (SEQ ID NO: 3); and

(b) a light chain variable region (VL) comprising a light chain complementarity determining region 1 (VLCDR1) amino acid sequence of RASQGISSWLA (SEQ ID NO: 14); a VLCDR2 amino acid sequence of AASSLQS (SEQ ID NO: 15); and a VLCDR3 amino acid sequence of QQGNSFPRT (SEQ ID NO: 16).

In one embodiment, antibody binding reduces the average hydrogen-deuterium exchange at residues 158-172 of SEQ ID NO: 105 to a greater extent than at residues 206-215 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105. In one embodiment, antibody binding reduces the average hydrogen-deuterium exchange at residues 158-172 of SEQ ID NO: 105 to a greater extent than at residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 368-387 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105, residues 368-387 of SEQ ID NO: 105, and residues 297-309 of SEQ ID NO: 105.

In one aspect, disclosed herein is an antibody molecule that binds to human CD73, wherein:

(i) the antibody molecule reduces hydrogen-deuterium exchange at one or more regions of a protein comprising the amino acid sequence of residues 27-547 of SEQ ID NO: 105 (e.g., a protein consisting of the amino acid sequence of SEQ ID NO: 171) when bound thereto, wherein the one or more regions are selected from the group consisting of residues 158-172, residues 206-215, residues 368-387, and residues 87-104 of SEQ ID NO: 105, wherein the region having the greatest reduction in the average hydrogen-deuterium exchange among the one or more regions is not residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises:

(a) a heavy chain variable region (VH) comprising a heavy chain complementarity determining region 1 (VHCDR1) amino acid sequence of X₁X₂YWS (SEQ ID NO: 90), wherein X₁ is R, G, or S, and

X₂ is Y or R; a VHCDR2 amino acid sequence of YIYX₁X₂GSTX₃YNPSLKS (SEQ ID NO: 91), wherein X₁ is G or S, X₂ is R, S, or T, and X₃ is N or K; and a VHCDR3 amino acid sequence of ESQESPYNNWFDP (SEQ ID NO: 3); and

(b) a light chain variable region (VL) comprising a light chain complementarity determining region 1 (VLCDR1) amino acid sequence of RASQGISSWLA (SEQ ID NO: 14); a VLCDR2 amino acid sequence of AASSLQS (SEQ ID NO: 15); and a VLCDR3 amino acid sequence of QQGNSFPRT (SEQ ID NO: 16).

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of more than, e.g., 0.02, 0.03, 0.04, 0.05, or 0.06 Da per residue at residues 368-387 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises:

(a) a heavy chain variable region (VH) comprising a heavy chain complementarity determining region 1 (VHCDR1) amino acid sequence of X₁X₂YWS (SEQ ID NO: 90), wherein X₁ is R, G, or S, and X₂ is Y or R; a VHCDR2 amino acid sequence of YIYX₁X₂GSTX₃YNPSLKS (SEQ ID NO: 91), wherein X₁ is G or S, X₂ is R, S, or T, and X₃ is N or K; and a VHCDR3 amino acid sequence of ESQESPYNNWFDP (SEQ ID NO: 3); and

(b) a light chain variable region (VL) comprising a light chain complementarity determining region 1 (VLCDR1) amino acid sequence of RASQGISSWLA (SEQ ID NO: 14); a VLCDR2 amino acid sequence of AASSLQS (SEQ ID NO: 15); and a VLCDR3 amino acid sequence of QQGNSFPRT (SEQ ID NO: 16).

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of less than, e.g., 0.05, 0.04, 0.03, or 0.02 Da per residue at residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises:

(a) a heavy chain variable region (VH) comprising a heavy chain complementarity determining region 1 (VHCDR1) amino acid sequence of X₁X₂YWS (SEQ ID NO: 90), wherein X₁ is R, G, or S, and X₂ is Y or R; a VHCDR2 amino acid sequence of YIYX₁X₂GSTX₃YNPSLKS (SEQ ID NO: 91), wherein X₁ is G or S, X₂ is R, S, or T, and X₃ is N or K; and a VHCDR3 amino acid sequence of ESQESPYNNWFDP (SEQ ID NO: 3); and

(b) a light chain variable region (VL) comprising a light chain complementarity determining region 1 (VLCDR1) amino acid sequence of RASQGISSWLA (SEQ ID NO: 14); a VLCDR2 amino acid sequence of AASSLQS (SEQ ID NO: 15); and a VLCDR3 amino acid sequence of QQGNSFPRT (SEQ ID NO: 16).

In one aspect, disclosed herein is an antibody molecule that binds to human CD73, wherein:

(i) the antibody molecule binds to at least one, two, three, or four residues within residues 158-172 of SEQ ID NO: 105, and/or at least one, two, three, four or five residues within residues 206-215 of SEQ ID NO: 105; and

(ii) the antibody molecule comprises:

(a) a heavy chain variable region (VH) comprising a heavy chain complementarity determining region 1 (VHCDR1) amino acid sequence of X₁X₂YWS (SEQ ID NO: 90), wherein X₁ is R, G, or S, and X₂ is Y or R; a VHCDR2 amino acid sequence of YIYX₁X₂GSTX₃YNPSLKS (SEQ ID NO: 91), wherein X₁ is G or S, X₂ is R, S, or T, and X₃ is N or K; and a VHCDR3 amino acid sequence of ESQESPYNNWFDP (SEQ ID NO: 3); and

(b) a light chain variable region (VL) comprising a light chain complementarity determining region 1 (VLCDR1) amino acid sequence of RASQGISSWLA (SEQ ID NO: 14); a VLCDR2 amino acid sequence of AASSLQS (SEQ ID NO: 15); and a VLCDR3 amino acid sequence of QQGNSFPRT (SEQ ID NO: 16).

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 368-387 of SEQ ID NO: 105 to a greater extent than at residues 158-172, residues 206-215, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises:

(a) a VH comprising a VHCDR1 amino acid sequence of SEQ ID NO: 61, a VHCDR2 amino acid sequence of SEQ ID NO: 60, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(b) a VH comprising a VHCDR1 amino acid sequence of SEQ ID NO: 4, a VHCDR2 amino acid sequence of SEQ ID NO: 26, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(c) a VH comprising a VHCDR1 amino acid sequence of SEQ ID NO: 4, a VHCDR2 amino acid sequence of SEQ ID NO: 2, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16; or

(d) a VH comprising a VHCDR1 amino acid sequence of SEQ ID NO: 163, a VHCDR2 amino acid sequence of SEQ ID NO: 162, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16.

In one embodiment, antibody binding reduces the average hydrogen-deuterium exchange at residues 368-387 of SEQ ID NO: 105 to a greater extent than at residues 158-172 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105. In one embodiment, antibody binding reduces the average hydrogen-deuterium exchange at residues 368-387 of SEQ ID NO: 105 to a greater extent than at residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 206-215 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 368-387 of SEQ ID NO: 105 to a greater extent than at residues 158-172 of SEQ ID NO: 105, residues 206-215 of SEQ ID NO: 105, and residues 297-309 of SEQ ID NO: 105.

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), reduces the average hydrogen-deuterium exchange at residues 158-172 of SEQ ID NO: 105 to a greater extent than at residues 206-215 of SEQ ID NO: 105, residues 368-387 of SEQ ID NO: 105, or residues 297-309 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises:

(a) a VH comprising a VHCDR1 amino acid sequence of SEQ ID NO: 61, a VHCDR2 amino acid sequence of SEQ ID NO: 60, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(b) a VH comprising a VHCDR1 amino acid sequence of SEQ ID NO: 4, a VHCDR2 amino acid sequence of SEQ ID NO: 26, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(c) a VH comprising a VHCDR1 amino acid sequence of SEQ ID NO: 4, a VHCDR2 amino acid sequence of SEQ ID NO: 2, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16; or

(d) a VH comprising a VHCDR1 amino acid sequence of SEQ ID NO: 163, a VHCDR2 amino acid sequence of SEQ ID NO: 162, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16.

In one embodiment, antibody binding reduces the average hydrogen-deuterium exchange at residues 158-172 of SEQ ID NO: 105 to a greater extent than at residues 206-215 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105. In one embodiment, antibody binding reduces the average hydrogen-deuterium exchange at residues 158-172 of SEQ ID NO: 105 to a greater extent than at residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 368-387 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 368-387 of SEQ ID NO: 105 and residues 297-309 of SEQ ID NO: 105. In one embodiment, antibody binding reduces the average hydrogen-deuterium exchange at residues 158-172 of SEQ ID NO: 105 to a greater extent than at residues 206-215 of SEQ ID NO: 105, residues 368-387 of SEQ ID NO: 105, and residues 297-309 of SEQ ID NO: 105.

In one aspect, disclosed herein is an antibody molecule that binds to human CD73, wherein:

(i) the antibody molecule reduces hydrogen-deuterium exchange at one or more regions of a protein comprising the amino acid sequence of residues 27-547 of SEQ ID NO: 105 (e.g., a protein consisting of the amino acid sequence of SEQ ID NO: 171) when bound thereto, wherein the one or more regions are selected from the group consisting of residues 158-172, residues 206-215, residues 368-387, and residues 87-104 of SEQ ID NO: 105, wherein the region having the greatest reduction in the average hydrogen-deuterium exchange among the one or more regions is not residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises:

(a) a VH comprising a VHCDR1 amino acid sequence of SEQ ID NO: 61, a VHCDR2 amino acid sequence of SEQ ID NO: 60, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(b) a VH comprising a VHCDR1 amino acid sequence of SEQ ID NO: 4, a VHCDR2 amino acid sequence of SEQ ID NO: 26, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(c) a VH comprising a VHCDR1 amino acid sequence of SEQ ID NO: 4, a VHCDR2 amino acid sequence of SEQ ID NO: 2, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16; or

(d) a VH comprising a VHCDR1 amino acid sequence of SEQ ID NO: 163, a VHCDR2 amino acid sequence of SEQ ID NO: 162, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16.

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of more than, e.g., 0.02, 0.03, 0.04, 0.05, or 0.06 Da per residue at residues 368-387 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises:

(a) a VH comprising a VHCDR1 amino acid sequence of SEQ ID NO: 61, a VHCDR2 amino acid sequence of SEQ ID NO: 60, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(b) a VH comprising a VHCDR1 amino acid sequence of SEQ ID NO: 4, a VHCDR2 amino acid sequence of SEQ ID NO: 26, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(c) a VH comprising a VHCDR1 amino acid sequence of SEQ ID NO: 4, a VHCDR2 amino acid sequence of SEQ ID NO: 2, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16; or

(d) a VH comprising a VHCDR1 amino acid sequence of SEQ ID NO: 163, a VHCDR2 amino acid sequence of SEQ ID NO: 162, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16.

In one aspect, disclosed herein is an antibody molecule that binds to human CD73, wherein:

(i) the antibody molecule, when bound to a protein comprising the amino acid sequence of residues 27-547 of SEQ ID NO: 105 (e.g., a protein consisting of the amino acid sequence of SEQ ID NO: 171), leads to a reduction in the average hydrogen-deuterium exchange of less than, e.g., 0.05, 0.04, 0.03, or 0.02 Da per residue at residues 206-215 of SEQ ID NO: 105, e.g., when the antibody molecule is tested as a bivalent antibody molecule using hydrogen deuterium-exchange mass spectrometry, e.g., as described in Example 2, e.g., hydrogen deuterium-exchange mass spectrometry conducted for 1-minute in-exchange at pH 7-8 (e.g., pH 7.5) and room temperature; and

(ii) the antibody molecule comprises:

(a) a VH comprising a VHCDR1 amino acid sequence of SEQ ID NO: 61, a VHCDR2 amino acid sequence of SEQ ID NO: 60, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(b) a VH comprising a VHCDR1 amino acid sequence of SEQ ID NO: 4, a VHCDR2 amino acid sequence of SEQ ID NO: 26, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(c) a VH comprising a VHCDR1 amino acid sequence of SEQ ID NO: 4, a VHCDR2 amino acid sequence of SEQ ID NO: 2, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16; or

(d) a VH comprising a VHCDR1 amino acid sequence of SEQ ID NO: 163, a VHCDR2 amino acid sequence of SEQ ID NO: 162, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16.

In one aspect, disclosed herein is an antibody molecule that binds to human CD73, wherein:

(i) the antibody molecule binds to at least one, two, three, or four residues within residues 158-172 of SEQ ID NO: 105, and/or at least one, two, three, four or five residues within residues 206-215 of SEQ ID NO: 105; and

(ii) the antibody molecule comprises:

(a) a VH comprising a VHCDR1 amino acid sequence of SEQ ID NO: 61, a VHCDR2 amino acid sequence of SEQ ID NO: 60, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(b) a VH comprising a VHCDR1 amino acid sequence of SEQ ID NO: 4, a VHCDR2 amino acid sequence of SEQ ID NO: 26, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(c) a VH comprising a VHCDR1 amino acid sequence of SEQ ID NO: 4, a VHCDR2 amino acid sequence of SEQ ID NO: 2, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16; or

(d) a VH comprising a VHCDR1 amino acid sequence of SEQ ID NO: 163, a VHCDR2 amino acid sequence of SEQ ID NO: 162, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16.

In one aspect, disclosed herein is an antibody molecule that binds to human CD73, wherein:

(i) the antibody molecule inhibits at least about 60%, 70%, 80%, or 90% of the enzymatic activity of membrane-bound human CD73, e.g., when the antibody molecule is tested as a bivalent antibody molecule using a modified Cell Titer Glo (CTG) assay, e.g., as described in Example 1; and (ii) the antibody molecule comprises a heavy chain variable region (VH) comprising a heavy chain complementarity determining region 3 (VHCDR3) amino acid sequence of GGLYGSGSYLSDFDL (SEQ ID NO: 37).

In one aspect, disclosed herein is an antibody molecule that binds to human CD73, wherein:

(i) the antibody molecule inhibits at least about 60%, 70%, 80%, or 90% of the enzymatic activity of membrane-bound human CD73, e.g., when the antibody molecule is tested as a bivalent antibody molecule using a modified Cell Titer Glo (CTG) assay, e.g., as described in Example 1; and

(ii) the antibody molecule comprises:

(a) a heavy chain variable region (VH) comprising a heavy chain complementarity determining region 1 (VHCDR1) amino acid sequence of X₁X₂AMS (SEQ ID NO: 88), wherein X₁ is R, Y, or S, and X₂ is Y or N; a VHCDR2 amino acid sequence of X₁IX₂GX₃GX₄X₅TYYADSVKG (SEQ ID NO: 89), wherein X₁ is A or S, X₂ is S or T, X₃ is S or T, X₄ is M, G, or S, and X₅ is N, S, L, or Y; and a VHCDR3 amino acid sequence of GGLYGSGSYLSDFDL (SEQ ID NO: 37); and

(b) a light chain variable region (VL) comprising a light chain complementarity determining region 1 (VLCDR1) amino acid sequence of RASQSVGSNLA (SEQ ID NO: 48); a VLCDR2 amino acid sequence of GASTRAT (SEQ ID NO: 49); and a VLCDR3 amino acid sequence of QQHNAFPYT (SEQ ID NO: 50).

In one aspect, disclosed herein is an antibody molecule that binds to human CD73, wherein:

(i) the antibody molecule inhibits at least about 60%, 70%, 80%, or 90% of the enzymatic activity of membrane-bound human CD73, e.g., when the antibody molecule is tested as a bivalent antibody molecule using a modified Cell Titer Glo (CTG) assay, e.g., as described in Example 1; and

(ii) the antibody molecule comprises:

(a) a VH comprising a VHCDR1 amino acid sequence of SEQ ID NO: 38, a VHCDR2 amino acid sequence of SEQ ID NO: 36, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(b) a VH comprising a VHCDR1 amino acid sequence of SEQ ID NO: 72, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(c) a VH comprising a VHCDR1 amino acid sequence of SEQ ID NO: 38, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(d) a VH comprising a VHCDR1 amino acid sequence of SEQ ID NO: 137, a VHCDR2 amino acid sequence of SEQ ID NO: 136, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;

(e) a VH comprising a VHCDR1 amino acid sequence of SEQ ID NO: 137, a VHCDR2 amino acid sequence of SEQ ID NO: 146, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; or

(f) a VH comprising a VHCDR1 amino acid sequence of SEQ ID NO: 137, a VHCDR2 amino acid sequence of SEQ ID NO: 154, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50.

In one aspect, disclosed herein is an antibody molecule that binds to human CD73, wherein:

(i) the antibody molecule inhibits at least about 60%, 70%, 80%, or 90% of the enzymatic activity of membrane-bound human CD73, e.g., when the antibody molecule is tested as a bivalent antibody molecule using a modified Cell Titer Glo (CTG) assay, e.g., as described in Example 1; and;

(ii) the antibody molecule comprises a heavy chain variable region (VH) comprising a heavy chain complementarity determining region 3 (VHCDR3) amino acid sequence of ESQESPYNNWFDP (SEQ ID NO: 3).

In one aspect, disclosed herein is an antibody molecule that binds to human CD73, wherein:

(i) the antibody molecule inhibits at least about 60%, 70%, 80%, or 90% of the enzymatic activity of membrane-bound human CD73, e.g., when the antibody molecule is tested as a bivalent antibody molecule using a modified Cell Titer Glo (CTG) assay, e.g., as described in Example 1; and

(ii) the antibody molecule comprises:

(a) a heavy chain variable region (VH) comprising a heavy chain complementarity determining region 1 (VHCDR1) amino acid sequence of X₁X₂YWS (SEQ ID NO: 90), wherein X₁ is R, G, or S, and X₂ is Y or R; a VHCDR2 amino acid sequence of YIYX₁X₂GSTX₃YNPSLKS (SEQ ID NO: 91), wherein X₁ is G or S, X₂ is R, S, or T, and X₃ is N or K; and a VHCDR3 amino acid sequence of ESQESPYNNWFDP (SEQ ID NO: 3); and/or

(b) a light chain variable region (VL) comprising a light chain complementarity determining region 1 (VLCDR1) amino acid sequence of RASQGISSWLA (SEQ ID NO: 14); a VLCDR2 amino acid sequence of AASSLQS (SEQ ID NO: 15); and a VLCDR3 amino acid sequence of QQGNSFPRT (SEQ ID NO: 16).

In one aspect, disclosed herein is an antibody molecule that binds to human CD73, wherein:

(i) the antibody molecule inhibits at least about 60%, 70%, 80%, or 90% of the enzymatic activity of membrane-bound human CD73, e.g., when the antibody molecule is tested as a bivalent antibody molecule using a modified Cell Titer Glo (CTG) assay, e.g., as described in Example 1; and;

(ii) the antibody molecule comprises:

(a) a VH comprising a VHCDR1 amino acid sequence of SEQ ID NO: 61, a VHCDR2 amino acid sequence of SEQ ID NO: 60, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(b) a VH comprising a VHCDR1 amino acid sequence of SEQ ID NO: 4, a VHCDR2 amino acid sequence of SEQ ID NO: 26, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;

(c) a VH comprising a VHCDR1 amino acid sequence of SEQ ID NO: 4, a VHCDR2 amino acid sequence of SEQ ID NO: 2, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16; or

(d) a VH comprising a VHCDR1 amino acid sequence of SEQ ID NO: 163, a VHCDR2 amino acid sequence of SEQ ID NO: 162, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16.

### Uses of the Anti-CD73 Antibody Molecules

The antibody molecules disclosed herein can modulate (e.g., enhance, stimulate, increase, inhibit, reduce or neutralize) one or more activities of CD73. In some embodiments, the antibody molecule results in one or more of: inhibiting or reducing the enzymatic activity of CD73; inhibiting or reducing the conversion of adenosine monophosphate (AMP) to adenosine; and increasing proliferation of anti-CD3/anti-CD28 stimulated T cells, e.g., CD4+ T cells, in the presence of adenosine monophosphate (AMP).

In some embodiments, the antibody molecule inhibits or reduces the enzymatic activity of CD73 (e.g., soluble human CD73 or membrane-bound human CD73), e.g., human CD73 mediated conversion of adenosine monophosphate (AMP) to adenosine, e.g., as measured by a method described herein, e.g., a malachite green (MG) phosphate assay or a modified Cell Titer Glo (CTG) assay, e.g., as described in Example 1.

In some embodiments, the antibody molecule inhibits at least about 60%, 70%, 80%, or 90% of the enzymatic activity of membrane-bound human CD73, e.g., when the antibody molecule is tested as a bivalent antibody molecule using a modified Cell Titer Glo (CTG) assay, e.g., as described in Example 1. In one embodiment, the antibody molecule inhibits at least about 60%, 70%, 80%, or 90% of the enzymatic activity of membrane-bound human CD73, e.g., when the antibody molecule is tested as a bivalent antibody molecule using a modified Cell Titer Glo (CTG) assay comprising the following steps:

(i) incubating a dose titration of the antibody molecule (e.g., 1000 ng/ml) with 20,000 cells/ml of a human cancer cell line expressing human CD73 (e.g., the human breast cancer cell line MDA-MB-231 or the human ovarian cancer cell line SKOV3) for 240 minutes at 37° C. in the presence of 100 μM AMP;

(ii) measuring disappearance of AMP using a modified Cell Titer Glo (CTG) assay as described in Example 1; and

(iii) calculating the percentage of inhibition mediated by the antibody molecule using time zero control as 100% inhibition and no antibody control as 0% inhibition.

In some embodiments, the antibody molecule increases proliferation of anti-CD3/anti-CD28 stimulated T cells, e.g., CD4+ T cells, in the presence of adenosine monophosphate (AMP), e.g., as measured by a method described herein, e.g., a CellTrace Violet (CTV) cell proliferation assay, e.g., an assay of Example 1.

In certain aspects, a method of modulating (e.g., stimulating or inhibiting) an immune response in a subject is provided. The method comprises administering to the subject an anti-CD73 antibody molecule disclosed herein, (e.g., a therapeutically effective amount of an anti-CD73 antibody molecule), alone or in combination with one or more agents or procedures (e.g., in combination with anti-tumor therapies, e.g., chemotherapies, radiation therapies, and/or other immunomodulatory agents), such that the immune response in the subject is modulated. In some embodiments, the antibody molecule inhibits, reduces, or neutralizes an immune response in a subject.

The subject can be a mammal, e.g., a monkey, a primate, preferably a higher primate, e.g., a human (e.g., a patient having, or at risk of having, a disorder described herein). In some embodiments, the subject is in need of enhancing an immune response, and in some embodiments, the subject is in need of inhibiting an immune response. In one embodiment, the subject has, or is at risk of, having a disorder described herein, e.g., a cancer as described herein. In certain embodiments, the subject is, or is at risk of being, immunocompromised. For example, the subject is undergoing or has undergone a chemotherapeutic treatment and/or radiation therapy.

In one aspect, a method of stimulating an immune response in a subject is provided. The method comprises administering to the subject an anti-CD73 antibody molecule described herein, e.g., a therapeutically effective amount of an anti-CD73 antibody molecule, alone or in combination with one or more agents or procedures.

In one aspect, a method of treating (e.g., one or more of reducing, inhibiting, or delaying progression) a cancer or a tumor in a subject is provided. The method comprises administering to the subject an anti-CD73 antibody molecule described herein, e.g., a therapeutically effective amount of an anti-CD73 antibody molecule, alone or in combination with one or more agents or procedures. In some embodiments, the antibody molecule is administered in combination with a second therapeutic agent or procedure. In some embodiments, the second therapeutic agent or procedure is chosen from one or more of chemotherapy, a targeted anti-cancer therapy, an oncolytic drug, a cytotoxic agent, an immune-based therapy, a cytokine, surgical procedure, a radiation procedure, an activator of a costimulatory molecule, an inhibitor of an inhibitory molecule (e.g., an inhibitor of a checkpoint inhibitor), a vaccine, or a cell therapy. In some embodiments, the second therapeutic agent is chosen from one or more of: 1) a protein kinase C (PKC) inhibitor; 2) a heat shock protein 90 (HSP90) inhibitor; 3) an inhibitor of a phosphoinositide 3-kinase (PI3K) and/or target of rapamycin (mTOR); 4) an inhibitor of cytochrome P450 (e.g., a CYP17 inhibitor or a 17alpha-Hydroxylase/C17-20 Lyase inhibitor); 5) an iron chelating agent; 6) an aromatase inhibitor; 7) an inhibitor of p53, e.g., an inhibitor of a p53/Mdm2 interaction; 8) an apoptosis inducer; 9) an angiogenesis inhibitor; 10) an aldosterone synthase inhibitor; 11) a smoothened (SMO) receptor inhibitor; 12) a prolactin receptor (PRLR) inhibitor; 13) a Wnt signaling inhibitor; 14) a CDK4/6 inhibitor; 15) a fibroblast growth factor receptor 2 (FGFR2)/fibroblast growth factor receptor 4 (FGFR4) inhibitor; 16) an inhibitor of macrophage colony-stimulating factor (M-CSF); 17) an inhibitor of one or more of c-KIT, histamine release, Flt3 (e.g., FLK2/STK1) or PKC; 18) an inhibitor of one or more of VEGFR-2 (e.g., FLK-1/KDR), PDGFRbeta, c-KIT or Raf kinase C; 19) a somatostatin agonist and/or a growth hormone release inhibitor; 20) an anaplastic lymphoma kinase (ALK) inhibitor; 21) an insulin-like growth factor 1 receptor (IGF-1R) inhibitor; 22) a P-Glycoprotein 1 inhibitor; 23) a vascular endothelial growth factor receptor (VEGFR) inhibitor; 24) a BCR-ABL kinase inhibitor; 25) an FGFR inhibitor; 26) an inhibitor of CYP11B2; 27) a HDM2 inhibitor, e.g., an inhibitor of the HDM2-p53 interaction; 28) an inhibitor of a tyrosine kinase; 29) an inhibitor of c-MET; 30) an inhibitor of JAK; 31) an inhibitor of DAC; 32) an inhibitor of 11β-hydroxylase; 33) an inhibitor of IAP; 34) an inhibitor of PIM kinase; 35) an inhibitor of Porcupine; 36) an inhibitor of BRAF, e.g., BRAF V600E or wild-type BRAF; 37) an inhibitor of HER3; 38) an inhibitor of MEK; or 39) an inhibitor of a lipid kinase.

In some embodiments, the anti-CD73 antibody molecule is administered in combination with a PD-1 inhibitor. In some embodiments, the PD-1 inhibitor is selected from the group consisting of PDR001, Nivolumab, Pembrolizumab, Pidilizumab, MEDI0680, REGN2810, TSR-042, PF-06801591, and AMP-224. In some embodiments, the PD-1 inhibitor is an anti-PD-1 antibody molecule. In some embodiments, the anti-PD-1 antibody molecule is administered, e.g., intravenously, at a dose of about 250 mg to 350 mg, about 350 mg to 450 mg, or about 450 mg to 550 mg, e.g., at a dose of about 300 mg or about 400 mg, e.g., once every three weeks (Q3W) or once every four weeks (Q4W), e.g., at a dose of about 300 mg Q3W, or at a dose of about 400 mg Q4W. In one embodiment, the anti-PD-1 antibody molecule is administered, e.g., by infusion, over a period of 30 minutes, or a period of up to 2 hours. In some embodiments, the anti-CD73 antibody molecule is administered, e.g., intravenously, at a dose of about 5 mg to 100 mg, about 100 mg to 500 mg, about 500 mg to 1000 mg, about 1000 mg to 1500 mg, about 1500 mg to 2000 mg, about 2000 mg to 2500 mg, about 2500 mg to 3000 mg, about 3000 mg to 3500 mg, or about 3500 mg to 4000 mg, e.g., at a dose of about 6 mg, about 20 mg, about 60 mg, about 200 mg, about 600 mg, about 1200 mg, about 2400 mg, about 3000 mg, or about 3600 mg, e.g., once every week (QW), once every two weeks (Q2W), or once every four weeks (Q4W), e.g., Q2W. In one embodiment, the anti-CD73 antibody molecule is administered, e.g., by infusion, over a period of 30 minutes, a period of 1 hour, or a period of up to 2 hours. In some embodiments, the anti-CD73 antibody molecule is administered, e.g., intravenously, at a dose of about 5 mg to 100 mg, e.g., 20 mg, Q2W, and the anti-PD-1 antibody molecule is administered, e.g., intravenously, at a dose of about 350 mg to 450 mg, e.g., 400 mg, Q4W. In some embodiments, the anti-CD73 antibody molecule is administered, e.g., intravenously, at a dose of about 5 mg to 100 mg, e.g., 60 mg, Q2W, and the anti-PD-1 antibody molecule is administered, e.g., intravenously, at a dose of about 350 mg to 450 mg, e.g., 400 mg, Q4W. In some embodiments, the anti-CD73 antibody molecule is administered, e.g., intravenously, at a dose of about 100 mg to 500 mg, e.g., 200 mg, Q2W, and the anti-PD-1 antibody molecule is administered, e.g., intravenously, at a dose of about 350 mg to 450 mg, e.g., 400 mg, Q4W. In some embodiments, the anti-CD73 antibody molecule is administered, e.g., intravenously, at a dose of about 500 mg to 1000 mg, e.g., 600 mg, Q2W, and the anti-PD-1 antibody molecule is administered, e.g., intravenously, at a dose of about 350 mg to 450 mg, e.g., 400 mg, Q4W. In some embodiments, the anti-CD73 antibody molecule is administered, e.g., intravenously, at a dose of about 1000 mg to 1500 mg, e.g., 1200 mg, Q2W, and the anti-PD-1 antibody molecule is administered, e.g., intravenously, at a dose of about 350 mg to 450 mg, e.g., 400 mg, Q4W. In some embodiments, the anti-CD73 antibody molecule is administered, e.g., intravenously, at a dose of about 2000 mg to 2500 mg, e.g., 2400 mg, Q2W, and the anti-PD-1 antibody molecule is administered, e.g., intravenously, at a dose of about 350 mg to 450 mg, e.g., 400 mg, Q4W. In some embodiments, the anti-CD73 antibody molecule is administered, e.g., intravenously, at a dose of about 3000 mg to 3500 mg, e.g., 3000 mg, Q2W, and the anti-PD-1 antibody molecule is administered, e.g., intravenously, at a dose of about 350 mg to 450 mg, e.g., 400 mg, Q4W. In some embodiments, the anti-CD73 antibody molecule is administered, e.g., intravenously, at a dose of about 3500 mg to 4000 mg, e.g., 3600 mg, Q2W, and the anti-PD-1 antibody molecule is administered, e.g., intravenously, at a dose of about 350 mg to 450 mg, e.g., 400 mg, Q4W.

In some embodiments, the anti-CD73 antibody molecule is administered in combination with a PD-L1 inhibitor. In some embodiments, the PD-L1 inhibitor is selected from the group consisting of FAZ053, Atezolizumab, Avelumab, Durvalumab, and BMS-936559.

In some embodiments, the anti-CD73 antibody molecule is administered in combination with a CTLA-4 inhibitor. In some embodiments, the CTLA-4 inhibitor is Ipilimumab or Tremelimumab.

In some embodiments, the anti-CD73 antibody molecule is administered in combination with a TIM-3 inhibitor. In some embodiments, the TIM-3 inhibitor is chosen from MGB453, TSR-022, or LY3321367.

In some embodiments, the anti-CD73 antibody molecule is administered in combination with a LAG-3 inhibitor. In some embodiments, the LAG-3 inhibitor is selected from the group consisting of LAG525, BMS-986016, TSR-033, MK-4280, and REGN3767.

In some embodiments, the anti-CD73 antibody molecule is administered in combination with a GITR agonist. In some embodiments, the GITR agonist is selected from the group consisting of GWN323 (Novartis), BMS-986156 (BMS), MK-4166 or MK-1248 (Merck), TRX518 (Leap Therapeutics), INCAGN1876 (Incyte/Agenus), AMG 228 (Amgen), and INBRX-110 (Inhibrx).

In some embodiments, the anti-CD73 antibody molecule is administered in combination with an anti-CD3 multispecific antibody molecule. In some embodiments, the anti-CD3 multispecific antibody molecule is an anti-CD3×anti-CD123 bispecific antibody molecule (e.g., XENP14045), or an anti-CD3×anti-CD20 bispecific antibody molecule (e.g., XENP13676).

In some embodiments, the anti-CD73 antibody molecule is administered in combination with a cytokine molecule. In some embodiments, the cytokine molecule is IL-15 complexed with a soluble form of IL-15 receptor alpha (IL-15Ra).

In some embodiments, the anti-CD73 antibody molecule is administered in combination with a STING agonist.

In some embodiments, the anti-CD73 antibody molecule is administered in combination with a macrophage colony-stimulating factor (M-CSF) inhibitor, optionally wherein the M-CSF inhibitor is MCS110.

In some embodiments, the anti-CD73 antibody molecule is administered in combination with a CSF-1R inhibitor, optionally wherein the CSF-1R inhibitor is BLZ945.

In some embodiments, the anti-CD73 antibody molecule is administered in combination with an inhibitor of indoleamine 2,3-dioxygenase (IDO) and/or tryptophan 2,3-dioxygenase (TDO).

In some embodiments, the anti-CD73 antibody molecule is administered in combination with a TGF-β inhibitor.

In some embodiments, the anti-CD73 antibody molecule is administered in combination with an adenosine A2AR antagonist. In some embodiments, the adenosine A2AR antagonist is selected from the group consisting of PBF509, CPI444, AZD4635, Vipadenant, GBV-2034, and AB928. In some embodiments, the adenosine A2AR antagonist is selected from the group consisting of 5-bromo-2,6-di-(1H-pyrazol-1-yl)pyrimidine-4-amine; (S)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; (R)-7-(5-methylfuran-2-yl)-3-((6-(((tetrahydrofuran-3-yl)oxy)methyl)pyridin-2-yl)methyl)-3H-[1,2,3]triazolo[4,5-c]pyrimidin-5-amine, or racemate thereof; 7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; and 6-(2-chloro-6-methylpyridin-4-yl)-5-(4-fluorophenyl)-1,2,4-triazin-3-amine In some embodiments, the adenosine A2AR antagonist is administered, e.g., orally, at a dose of about 20 mg to 60 mg, about 60 mg to 100 mg, about 100 mg to 140 mg, about 140 mg to 180 mg, about 180 mg to 220 mg, about 220 mg to 260 mg, about 260 mg to 300 mg, about 300 mg to 340 mg, about 340 mg to 380 mg, about 380 mg to 480 mg, about 480 mg to 580 mg, or about 580 mg to 680 mg, e.g., at a dose of about 40 mg, about 80 mg, about 160 mg, about 320 mg, about 480 mg, or about 620 mg, e.g., once a day (QD), twice a day (BID), or three times a day (TID), e.g., BID. In some embodiments, the anti-CD73 antibody molecule is administered, e.g., intravenously, at a dose of about 5 mg to 100 mg, about 100 mg to 500 mg, about 500 mg to 1000 mg, about 1000 mg to 1500 mg, about 1500 mg to 2000 mg, about 2000 mg to 2500 mg, about 2500 mg to 3000 mg, about 3000 mg to 3500 mg, or about 3500 mg to 4000 mg, e.g., at a dose of about 6 mg, about 20 mg, about 60 mg, about 200 mg, about 600 mg, about 1200 mg, about 2400 mg, about 3000 mg, or about 3600 mg, e.g., once every week (QW), once every two weeks (Q2W), or once every four weeks (Q4W), e.g., Q2W. In some embodiments, the anti-CD73 antibody molecule is administered, e.g., intravenously, at a dose of about 5 mg to 100 mg, e.g., 20 mg, Q2W, and the adenosine A2AR antagonist is administered, e.g., orally, at a dose of about 20 mg to 60 mg, e.g., 40 mg, BID. In some embodiments, the anti-CD73 antibody molecule is administered, e.g., intravenously, at a dose of about 5 mg to 100 mg, e.g., 20 mg, Q2W, and the adenosine A2AR antagonist is administered, e.g., orally, at a dose of about 60 mg to 100 mg, e.g., 80 mg, BID. In some embodiments, the anti-CD73 antibody molecule is administered, e.g., intravenously, at a dose of about 5 mg to 100 mg, e.g., 20 mg, Q2W, and the adenosine A2AR antagonist is administered, e.g., orally, at a dose of about 140 mg to 180 mg, e.g., 160 mg, BID. In some embodiments, the anti-CD73 antibody molecule is administered, e.g., intravenously, at a dose of about 5 mg to 100 mg, e.g., 20 mg, Q2W, and the adenosine A2AR antagonist is administered, e.g., orally, at a dose of about 300 mg to 340 mg, e.g., 320 mg, BID. In some embodiments, the anti-CD73 antibody molecule is administered, e.g., intravenously, at a dose of about 5 mg to 100 mg, e.g., 60 mg, Q2W, and the adenosine A2AR antagonist is administered, e.g., orally, at a dose of about 20 mg to 60 mg, e.g., 40 mg, BID. In some embodiments, the anti-CD73 antibody molecule is administered, e.g., intravenously, at a dose of about 5 mg to 100 mg, e.g., 60 mg, Q2W, and the adenosine A2AR antagonist is administered, e.g., orally, at a dose of about 60 mg to 100 mg, e.g., 80 mg, BID. In some embodiments, the anti-CD73 antibody molecule is administered, e.g., intravenously, at a dose of about 5 mg to 100 mg, e.g., 60 mg, Q2W, and the adenosine A2AR antagonist is administered, e.g., orally, at a dose of about 140 mg to 180 mg, e.g., 160 mg, BID. In some embodiments, the anti-CD73 antibody molecule is administered, e.g., intravenously, at a dose of about 5 mg to 100 mg, e.g., 60 mg, Q2W, and the adenosine A2AR antagonist is administered, e.g., orally, at a dose of about 300 mg to 340 mg, e.g., 320 mg, BID. In some embodiments, the anti-CD73 antibody molecule is administered, e.g., intravenously, at a dose of about 100 mg to 500 mg, e.g., 200 mg, Q2W, and the adenosine A2AR antagonist is administered, e.g., orally, at a dose of about 20 mg to 60 mg, e.g., 40 mg, BID. In some embodiments, the anti-CD73 antibody molecule is administered, e.g., intravenously, at a dose of about 100 mg to 500 mg, e.g., 200 mg, Q2W, and the adenosine A2AR antagonist is administered, e.g., orally, at a dose of about 60 mg to 100 mg, e.g., 80 mg, BID. In some embodiments, the anti-CD73 antibody molecule is administered, e.g., intravenously, at a dose of about 100 mg to 500 mg, e.g., 200 mg, Q2W, and the adenosine A2AR antagonist is administered, e.g., orally, at a dose of about 140 mg to 180 mg, e.g., 160 mg, BID. In some embodiments, the anti-CD73 antibody molecule is administered, e.g., intravenously, at a dose of about 100 mg to 500 mg, e.g., 200 mg, Q2W, and the adenosine A2AR antagonist is administered, e.g., orally, at a dose of about 300 mg to 340 mg, e.g., 320 mg, BID. In some embodiments, the anti-CD73 antibody molecule is administered, e.g., intravenously, at a dose of about 500 mg to 1000 mg, e.g., 600 mg, Q2W, and the adenosine A2AR antagonist is administered, e.g., orally, at a dose of about 20 mg to 60 mg, e.g., 40 mg, BID. In some embodiments, the anti-CD73 antibody molecule is administered, e.g., intravenously, at a dose of about 500 mg to 1000 mg, e.g., 600 mg, Q2W, and the adenosine A2AR antagonist is administered, e.g., orally, at a dose of about 60 mg to 100 mg, e.g., 80 mg, BID. In some embodiments, the anti-CD73 antibody molecule is administered, e.g., intravenously, at a dose of about 500 mg to 1000 mg, e.g., 600 mg, Q2W, and the adenosine A2AR antagonist is administered, e.g., orally, at a dose of about 140 mg to 180 mg, e.g., 160 mg, BID. In some embodiments, the anti-CD73 antibody molecule is administered, e.g., intravenously, at a dose of about 500 mg to 1000 mg, e.g., 600 mg, Q2W, and the adenosine A2AR antagonist is administered, e.g., orally, at a dose of about 300 mg to 340 mg, e.g., 320 mg, BID. In some embodiments, the anti-CD73 antibody molecule is administered, e.g., intravenously, at a dose of about 1000 mg to 1500 mg, e.g., 1200 mg, Q2W, and the adenosine A2AR antagonist is administered, e.g., orally, at a dose of about 20 mg to 60 mg, e.g., 40 mg, BID. In some embodiments, the anti-CD73 antibody molecule is administered, e.g., intravenously, at a dose of about 1000 mg to 1500 mg, e.g., 1200 mg, Q2W, and the adenosine A2AR antagonist is administered, e.g., orally, at a dose of about 60 mg to 100 mg, e.g., 80 mg, BID. In some embodiments, the anti-CD73 antibody molecule is administered, e.g., intravenously, at a dose of about 1000 mg to 1500 mg, e.g., 1200 mg, Q2W, and the adenosine A2AR antagonist is administered, e.g., orally, at a dose of about 140 mg to 180 mg, e.g., 160 mg, BID. In some embodiments, the anti-CD73 antibody molecule is administered, e.g., intravenously, at a dose of about 1000 mg to 1500 mg, e.g., 1200 mg, Q2W, and the adenosine A2AR antagonist is administered, e.g., orally, at a dose of about 300 mg to 340 mg, e.g., 320 mg, BID. In some embodiments, the anti-CD73 antibody molecule is administered, e.g., intravenously, at a dose of about 2000 mg to 2500 mg, e.g., 2400 mg, Q2W, and the adenosine A2AR antagonist is administered, e.g., orally, at a dose of about 20 mg to 60 mg, e.g., 40 mg, BID. In some embodiments, the anti-CD73 antibody molecule is administered, e.g., intravenously, at a dose of about 2000 mg to 2500 mg, e.g., 2400 mg, Q2W, and the adenosine A2AR antagonist is administered, e.g., orally, at a dose of about 60 mg to 100 mg, e.g., 80 mg, BID. In some embodiments, the anti-CD73 antibody molecule is administered, e.g., intravenously, at a dose of about 2000 mg to 2500 mg, e.g., 2400 mg, Q2W, and the adenosine A2AR antagonist is administered, e.g., orally, at a dose of about 140 mg to 180 mg, e.g., 160 mg, BID. In some embodiments, the anti-CD73 antibody molecule is administered, e.g., intravenously, at a dose of about 2000 mg to 2500 mg, e.g., 2400 mg, Q2W, and the adenosine A2AR antagonist is administered, e.g., orally, at a dose of about 300 mg to 340 mg, e.g., 320 mg, BID. In some embodiments, the anti-CD73 antibody molecule is administered, e.g., intravenously, at a dose of about 2000 mg to 2500 mg, e.g., 2400 mg, Q2W, and the adenosine A2AR antagonist is administered, e.g., orally, at a dose of about 480 mg to 580 mg, e.g., 480 mg, BID. In some embodiments, the anti-CD73 antibody molecule is administered, e.g., intravenously, at a dose of about 2000 mg to 2500 mg, e.g., 2400 mg, Q2W, and the adenosine A2AR antagonist is administered, e.g., orally, at a dose of about 580 mg to 680 mg, e.g., 620 mg, BID. In some embodiments, the anti-CD73 antibody molecule is administered, e.g., intravenously, at a dose of about 3000 mg to 3500 mg, e.g., 3000 mg, Q2W, and the adenosine A2AR antagonist is administered, e.g., orally, at a dose of about 300 mg to 340 mg, e.g., 320 mg, BID. In some embodiments, the anti-CD73 antibody molecule is administered, e.g., intravenously, at a dose of about 3000 mg to 3500 mg, e.g., 3000 mg, Q2W, and the adenosine A2AR antagonist is administered, e.g., orally, at a dose of about 480 mg to 580 mg, e.g., 480 mg, BID. In some embodiments, the anti-CD73 antibody molecule is administered, e.g., intravenously, at a dose of about 3000 mg to 3500 mg, e.g., 3000 mg, Q2W, and the adenosine A2AR antagonist is administered, e.g., orally, at a dose of about 580 mg to 680 mg, e.g., 620 mg, BID. In some embodiments, the anti-CD73 antibody molecule is administered, e.g., intravenously, at a dose of about 3500 mg to 4000 mg, e.g., 3600 mg, Q2W, and the adenosine A2AR antagonist is administered, e.g., orally, at a dose of about 300 mg to 340 mg, e.g., 320 mg, BID. In some embodiments, the anti-CD73 antibody molecule is administered, e.g., intravenously, at a dose of about 3500 mg to 4000 mg, e.g., 3600 mg, Q2W, and the adenosine A2AR antagonist is administered, e.g., orally, at a dose of about 480 mg to 580 mg, e.g., 480 mg, BID. In some embodiments, the anti-CD73 antibody molecule is administered, e.g., intravenously, at a dose of about 3500 mg to 4000 mg, e.g., 3600 mg, Q2W, and the adenosine A2AR antagonist is administered, e.g., orally, at a dose of about 580 mg to 680 mg, e.g., 620 mg, BID.

In some embodiments, the anti-CD73 antibody molecule is administered in combination with a PD-1 inhibitor and an adenosine A2AR antagonist. In some embodiments, the anti-CD73 antibody molecule is administered, e.g., intravenously, at a dose of about 5 mg to 100 mg, about 100 mg to 500 mg, about 500 mg to 1000 mg, about 1000 mg to 1500 mg, about 1500 mg to 2000 mg, about 2000 mg to 2500 mg, about 2500 mg to 3000 mg, about 3000 mg to 3500 mg, or about 3500 mg to 4000 mg, e.g., at a dose of about 6 mg, about 20 mg, about 60 mg, about 200 mg, about 600 mg, about 1200 mg, about 2400 mg, about 3000 mg, or about 3600 mg, e.g., once every week (QW), once every two weeks (Q2W), or once every four weeks (Q4W), e.g., Q2W. In some embodiments, the PD-1 inhibitor is an anti-PD-1 antibody molecule. In some embodiments, the anti-PD-1 antibody molecule is administered, e.g., intravenously, at a dose of about 250 mg to 350 mg, about 350 mg to 450 mg, or about 450 mg to 550 mg, e.g., at a dose of about 300 mg or about 400 mg, e.g., once every three weeks (Q3W) or once every four weeks (Q4W), e.g., at a dose of about 300 mg Q3W, or at a dose of about 400 mg Q4W. In some embodiments, the adenosine A2AR antagonist is administered, e.g., orally, at a dose of about 20 mg to 60 mg, about 60 mg to 100 mg, about 100 mg to 140 mg, about 140 mg to 180 mg, about 180 mg to 220 mg, about 220 mg to 260 mg, about 260 mg to 300 mg, about 300 mg to 340 mg, about 340 mg to 380 mg, about 380 mg to 480 mg, about 480 mg to 580 mg, or about 580 mg to 680 mg, e.g., at a dose of about 40 mg, about 80 mg, about 160 mg, about 320 mg, about 480 mg, or about 620 mg, e.g., once a day (QD), twice a day (BID), or three times a day (TID), e.g., BID. In some embodiments, the anti-CD73 antibody molecule is administered, e.g., intravenously, at a dose of about 5 mg to 100 mg, e.g., 20 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 20 mg to 60 mg, e.g., 40 mg, BID. In some embodiments, the anti-CD73 antibody molecule is administered, e.g., intravenously, at a dose of about 5 mg to 100 mg, e.g., 20 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 60 mg to 100 mg, e.g., 80 mg, BID. In some embodiments, the anti-CD73 antibody molecule is administered, e.g., intravenously, at a dose of about 5 mg to 100 mg, e.g., 20 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 140 mg to 180 mg, e.g., 160 mg, BID. In some embodiments, the anti-CD73 antibody molecule is administered, e.g., intravenously, at a dose of about 5 mg to 100 mg, e.g., 20 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 300 mg to 340 mg, e.g., 320 mg, BID. In some embodiments, the anti-CD73 antibody molecule is administered, e.g., intravenously, at a dose of about 5 mg to 100 mg, e.g., 60 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 20 mg to 60 mg, e.g., 40 mg, BID. In some embodiments, the anti-CD73 antibody molecule is administered, e.g., intravenously, at a dose of about 5 mg to 100 mg, e.g., 60 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 60 mg to 100 mg, e.g., 80 mg, BID. In some embodiments, the anti-CD73 antibody molecule is administered, e.g., intravenously, at a dose of about 5 mg to 100 mg, e.g., 60 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 140 mg to 180 mg, e.g., 160 mg, BID. In some embodiments, the anti-CD73 antibody molecule is administered, e.g., intravenously, at a dose of about 5 mg to 100 mg, e.g., 60 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 300 mg to 340 mg, e.g., 320 mg, BID. In some embodiments, the anti-CD73 antibody molecule is administered, e.g., intravenously, at a dose of about 100 mg to 500 mg, e.g., 200 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 20 mg to 60 mg, e.g., 40 mg, BID. In some embodiments, the anti-CD73 antibody molecule is administered, e.g., intravenously, at a dose of about 100 mg to 500 mg, e.g., 200 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 60 mg to 100 mg, e.g., 80 mg, BID. In some embodiments, the anti-CD73 antibody molecule is administered, e.g., intravenously, at a dose of about 100 mg to 500 mg, e.g., 200 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 140 mg to 180 mg, e.g., 160 mg, BID. In some embodiments, the anti-CD73 antibody molecule is administered, e.g., intravenously, at a dose of about 100 mg to 500 mg, e.g., 200 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 300 mg to 340 mg, e.g., 320 mg, BID. In some embodiments, the anti-CD73 antibody molecule is administered, e.g., intravenously, at a dose of about 500 mg to 1000 mg, e.g., 600 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 20 mg to 60 mg, e.g., 40 mg, BID. In some embodiments, the anti-CD73 antibody molecule is administered, e.g., intravenously, at a dose of about 500 mg to 1000 mg, e.g., 600 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 60 mg to 100 mg, e.g., 80 mg, BID. In some embodiments, the anti-CD73 antibody molecule is administered, e.g., intravenously, at a dose of about 500 mg to 1000 mg, e.g., 600 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 140 mg to 180 mg, e.g., 160 mg, BID. In some embodiments, the anti-CD73 antibody molecule is administered, e.g., intravenously, at a dose of about 500 mg to 1000 mg, e.g., 600 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 300 mg to 340 mg, e.g., 320 mg, BID. In some embodiments, the anti-CD73 antibody molecule is administered, e.g., intravenously, at a dose of about 1000 mg to 1500 mg, e.g., 1200 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 20 mg to 60 mg, e.g., 40 mg, BID. In some embodiments, the anti-CD73 antibody molecule is administered, e.g., intravenously, at a dose of about 1000 mg to 1500 mg, e.g., 1200 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 60 mg to 100 mg, e.g., 80 mg, BID. In some embodiments, the anti-CD73 antibody molecule is administered, e.g., intravenously, at a dose of about 1000 mg to 1500 mg, e.g., 1200 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 140 mg to 180 mg, e.g., 160 mg, BID. In some embodiments, the anti-CD73 antibody molecule is administered, e.g., intravenously, at a dose of about 1000 mg to 1500 mg, e.g., 1200 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 300 mg to 340 mg, e.g., 320 mg, BID. In some embodiments, the anti-CD73 antibody molecule is administered, e.g., intravenously, at a dose of about 2000 mg to 2500 mg, e.g., 2400 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 20 mg to 60 mg, e.g., 40 mg, BID. In some embodiments, the anti-CD73 antibody molecule is administered, e.g., intravenously, at a dose of about 2000 mg to 2500 mg, e.g., 2400 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 60 mg to 100 mg, e.g., 80 mg, BID. In some embodiments, the anti-CD73 antibody molecule is administered, e.g., intravenously, at a dose of about 2000 mg to 2500 mg, e.g., 2400 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 140 mg to 180 mg, e.g., 160 mg, BID. In some embodiments, the anti-CD73 antibody molecule is administered, e.g., intravenously, at a dose of about 2000 mg to 2500 mg, e.g., 2400 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 300 mg to 340 mg, e.g., 320 mg, BID. In some embodiments, the anti-CD73 antibody molecule is administered, e.g., intravenously, at a dose of about 2000 mg to 2500 mg, e.g., 2400 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 480 mg to 580 mg, e.g., 480 mg, BID. In some embodiments, the anti-CD73 antibody molecule is administered, e.g., intravenously, at a dose of about 2000 mg to 2500 mg, e.g., 2400 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 580 mg to 680 mg, e.g., 620 mg, BID. In some embodiments, the anti-CD73 antibody molecule is administered, e.g., intravenously, at a dose of about 3000 mg to 3500 mg, e.g., 3000 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 300 mg to 340 mg, e.g., 320 mg, BID. In some embodiments, the anti-CD73 antibody molecule is administered, e.g., intravenously, at a dose of about 3000 mg to 3500 mg, e.g., 3000 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 480 mg to 580 mg, e.g., 480 mg, BID. In some embodiments, the anti-CD73 antibody molecule is administered, e.g., intravenously, at a dose of about 3000 mg to 3500 mg, e.g., 3000 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 580 mg to 680 mg, e.g., 620 mg, BID. In some embodiments, the anti-CD73 antibody molecule is administered, e.g., intravenously, at a dose of about 3500 mg to 4000 mg, e.g., 3600 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 300 mg to 340 mg, e.g., 320 mg, BID. In some embodiments, the anti-CD73 antibody molecule is administered, e.g., intravenously, at a dose of about 3500 mg to 4000 mg, e.g., 3600 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 480 mg to 580 mg, e.g., 480 mg, BID. In some embodiments, the anti-CD73 antibody molecule is administered, e.g., intravenously, at a dose of about 3500 mg to 4000 mg, e.g., 3600 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 580 mg to 680 mg, e.g., 620 mg, BID.

In some embodiments, the anti-CD73 antibody molecule is administered in combination with a PD-L1 inhibitor and an adenosine A2AR antagonist.

In some embodiments, the anti-CD73 antibody molecule is administered in combination with a chimeric antigen receptor (CAR) T-cell therapy. In some embodiments, the CAR T-cell therapy is CTL019.

In some embodiments, the anti-CD73 antibody molecule is administered in combination with one or more agents disclosed in Table 18, e.g., one or more of: 1) a protein kinase C (PKC) inhibitor; 2) a heat shock protein 90 (HSP90) inhibitor; 3) an inhibitor of a phosphoinositide 3-kinase (PI3K) and/or target of rapamycin (mTOR); 4) an inhibitor of cytochrome P450 (e.g., a CYP17 inhibitor or a 17alpha-Hydroxylase/C17-20 Lyase inhibitor); 5) an iron chelating agent; 6) an aromatase inhibitor; 7) an inhibitor of p53, e.g., an inhibitor of a p53/Mdm2 interaction; 8) an apoptosis inducer; 9) an angiogenesis inhibitor; 10) an aldosterone synthase inhibitor; 11) a smoothened (SMO) receptor inhibitor; 12) a prolactin receptor (PRLR) inhibitor; 13) a Wnt signaling inhibitor; 14) a CDK4/6 inhibitor; 15) a fibroblast growth factor receptor 2 (FGFR2)/fibroblast growth factor receptor 4 (FGFR4) inhibitor; 16) an inhibitor of macrophage colony-stimulating factor (M-CSF); 17) an inhibitor of one or more of c-KIT, histamine release, Flt3 (e.g., FLK2/STK1) or PKC; 18) an inhibitor of one or more of VEGFR-2 (e.g., FLK-1/KDR), PDGFRbeta, c-KIT or Raf kinase C; 19) a somatostatin agonist and/or a growth hormone release inhibitor; 20) an anaplastic lymphoma kinase (ALK) inhibitor; 21) an insulin-like growth factor 1 receptor (IGF-1R) inhibitor; 22) a P-Glycoprotein 1 inhibitor; 23) a vascular endothelial growth factor receptor (VEGFR) inhibitor; 24) a BCR-ABL kinase inhibitor; 25) an FGFR inhibitor; 26) an inhibitor of CYP11B2; 27) a HDM2 inhibitor, e.g., an inhibitor of the HDM2-p53 interaction; 28) an inhibitor of a tyrosine kinase; 29) an inhibitor of c-MET; 30) an inhibitor of JAK; 31) an inhibitor of DAC; 32) an inhibitor of 11β-hydroxylase; 33) an inhibitor of IAP; 34) an inhibitor of PIM kinase; 35) an inhibitor of Porcupine; 36) an inhibitor of BRAF, e.g., BRAF V600E or wild-type BRAF; 37) an inhibitor of HER3; 38) an inhibitor of MEK; or 39) an inhibitor of a lipid kinase, e.g., as described herein and in Table 18.

In certain embodiments, the cancer treated with the anti-CD73 antibody molecule, alone or in combination with a second therapeutic agent or procedure, includes but is not limited to, a solid tumor, a hematological cancer (e.g., leukemia, lymphoma, myeloma, e.g., multiple myeloma), and a metastatic lesion. In some embodiments, the cancer is chosen from lung cancer (e.g., non-small cell lung cancer), pancreas cancer (e.g., pancreatic ductal adenocarcinoma), breast cancer (e.g., triple-negative breast cancer), melanoma, head and neck cancer (e.g., squamous head and neck cancer), colorectal cancer (e.g., microsatellite stable (MSS) colorectal cancer), ovarian cancer, or renal cancer (e.g., renal cell carcinoma). In certain embodiments, the anti-CD73 antibody molecule, used alone or in combination with a second therapeutic agent or procedure, controls tumor growth, reduces metastasis, and/or improves survival.

In certain embodiments, the antibody molecule is administered at a dose of about 100 mg to 1600 mg, about 100 mg to 1400 mg, about 100 mg to 1200 mg, about 100 mg to 1000 mg, about 100 mg to 800 mg, about 100 mg to 600 mg, about 100 mg to 400 mg, about 100 mg to 200 mg, or about 100 mg, about 180 mg, or about 200 mg, e.g., once every two weeks. In one embodiment, the antibody molecule is administered at a dose of at least about 180 mg once every two weeks.

In certain embodiments, the antibody molecule is administered, e.g., intravenously, at a dose of about 5 mg to 100 mg, about 100 mg to 500 mg, about 500 mg to 1000 mg, about 1000 mg to 1500 mg, about 1500 mg to 2000 mg, about 2000 mg to 2500 mg, about 2500 mg to 3000 mg, about 3000 mg to 3500 mg, or about 3500 mg to 4000 mg, e.g., once every week (QW), once every two weeks (Q2W), or once every four weeks (Q4W). In certain embodiments, the antibody molecule is administered, e.g., intravenously, at a dose of about 6 mg, about 20 mg, about 60 mg, about 200 mg, about 600 mg, about 1200 mg, about 2400 mg, about 3000 mg, or about 3600 mg, e.g., QW, Q2W, or Q4W. In certain embodiments, the antibody molecule is administered, e.g., intravenously, at a dose of about 60 mg Q2W. In certain embodiments, the antibody molecule is administered, e.g., intravenously, at a dose of about 200 mg Q2W. In certain embodiments, the antibody molecule is administered, e.g., intravenously, at a dose of about 600 mg Q2W. In certain embodiments, the antibody molecule is administered, e.g., intravenously, at a dose of about 1200 mg Q2W. In certain embodiments, the antibody molecule is administered, e.g., intravenously, at a dose of about 2400 mg Q2W. In certain embodiments, the antibody molecule is administered, e.g., intravenously, at a dose of about 3000 mg Q2W. In certain embodiments, the antibody molecule is administered, e.g., intravenously, at a dose of about 3600 mg Q2W.

Still further, this disclosure provides methods of enhancing an immune response to an antigen in a subject, comprising administering to the subject: (i) the antigen; and (ii) an anti-CD73 antibody molecule disclosed herein, such that an immune response to the antigen in the subject is enhanced. The antigen can be, for example, a tumor antigen, a viral antigen, a bacterial antigen or an antigen from a pathogen.

The anti-CD73 antibody molecule can be administered to the subject systemically (e.g., orally, parenterally, subcutaneously, intravenously, rectally, intramuscularly, intraperitoneally, intranasally, transdermally, or by inhalation or intracavitary installation), or locally. In one embodiment, the anti-CD73 antibody molecule is administered intravenously.

The anti-CD73 antibody molecule can be used alone in unconjugated form, or can be bound to a substance, e.g., a cytotoxic agent or moiety (e.g., a therapeutic drug; a compound emitting radiation; molecules of plant, fungal, or bacterial origin; or a biological protein (e.g., a protein toxin) or particle (e.g., a recombinant viral particle, e.g., via a viral coat protein). For example, the anti-CD73 antibody can be coupled to a radioactive isotope such as an α-, β-, or γ-emitter, or a β- and γ-emitter.

Dosages and therapeutic regimens of the anti-CD73 antibody molecule can be determined by a skilled artisan.

In another aspect, a method of detecting CD73 in a biological sample or in a subject is provided. In one embodiment, the method comprises (i) contacting the sample or the subject (and optionally, a reference sample or subject) with an anti-CD73 antibody molecule described herein under conditions that allow interaction of the antibody molecule and CD73 to occur, and (ii) detecting formation of a complex between the antibody molecule and the sample or the subject (and optionally, the reference sample or subject).

The antibody molecules described herein are preferred for use in the methods described herein, although other anti-CD73 antibodies can be used instead, or in combination with an anti-CD73 antibody molecule of the invention.

### BRIEF DESCRIPTION OF THE DRAWINGS

FIG. 1 is a graph showing the binding of anti-CD73 antibodies or an isotype control antibody to CD8+ T cells, measured by flow cytometry. MFI values are plotted against antibody concentrations. The antibodies tested are the anti-CD73 antibodies 350, 356, 358, 373, 374, 377, and 379, as well as an isotype control antibody, all expressed in the .B format.

FIGS. 2A and 2B are graphs showing results from a malachite green inorganic phosphate assay testing the ability of anti-CD73 antibody to inhibit human CD73-medicated conversion of adenosine monophosphate (AMP) to adenosine. Rate of inorganic phosphate (Pi) release is plotted against AMP concentrations tested. In FIG. 2A, recombinant human CD73 was incubated with the substrate AMP with buffer alone (“hCD73 Km”) or in the presence of an IgG1 isotype control antibody (“ISO.C”). In FIG. 2B, recombinant CD73 was incubated with AMP with buffer alone (“hCD73 Km”) or in the presence of anti-CD73 antibody 350.0 at indicated concentrations (“1 μg/ml 350.C,” “0.3 μg/ml 350.C,” or “0.1 μg/ml 350.C”).

FIGS. 3A, 3B, and 3C are graphs showing results from a malachite green (MG) phosphate assay testing the ability of anti-CD73 antibody to inhibit the enzymatic activity of recombinant soluble human or cynomolgus CD73. % INH phosphate formation is plotted against anti-CD73 antibody concentrations for the studies using recombinant human CD73 (FIGS. 3A and 3B) or cynomolgus CD73 (FIG. 3C). The antibodies tested are the anti-CD73 antibodies 350, 356, 373, and 374, expressed in either the .A or .B format.

FIG. 4 is a graph showing results from a modified Cell Titer Glo (CTG) assay examining the enzyme inhibition activity of anti-CD73 antibodies against CD73 shed from a breast cancer cell line MDA-MB-231. % INH is plotted against a range of anti-CD73 antibody concentrations. The antibodies tested are the anti-CD73 antibodies 350 and 373, expressed in either the .A or .B format, an IgG4 S228P isotype control antibody (“ISO.A”), and an IgG4 S228P/L235E isotype control antibody (“ISO.B”).

FIG. 5 is a graph showing results from a modified Cell Titer Glo (CTG) assay examining the ability of anti-CD73 antibodies to inhibit the enzymatic activity of CD73 in the serum from a pancreatic cancer patient. % INH AMP conversion is plotted against anti-CD73 antibody concentrations. The anti-CD73 antibodies tested are 350, 356, 358, 373, 374, 377, and 379, all expressed in the .B format.

FIG. 6 is a graph showing inhibition of CD73 expressed on the surface of a breast cancer cell line MDA-MB-231, measured using a malachite green phosphate assay. % INH AMP conversion is plotted over a range of anti-CD73 antibody concentrations. The anti-CD73 antibodies tested are 350, 356, 358, 373, 374, 377, and 379, all in the .B format.

FIGS. 7A, 7B, 7C, and 7D are bar graphs showing results from a modified Cell Titer Glo (CTG) assay testing the ability of anti-CD73 antibodies to inhibit the enzymatic activity of CD73 expressed on the surface of a human breast cancer cell line MDA-MB-231 (FIGS. 7A and 7C) or a murine breast cancer cell line 4T1 (FIGS. 7B and 7D). The y-axis shows percentage of inhibition relative to the no antibody (full conversion) control and the time zero (no conversion) control. For each antibody, the bars from left to right represent 10, 3, 1, and 0.3 μg/ml doses. The antibodies tested are the Lineage 1 antibodies 918, 350, 356, and 358 (FIGS. 7A and 7B) and the Lineage 3 antibodies 930, 373, 374, 376, 377, and 379 (FIGS. 7C and 7D).

FIGS. 8A and 8B are graphs showing the enzyme inhibition activity of anti-CD73 antibodies against surface CD73 expressed on a human breast cancer cell line MDA-MB-231 or a human ovarian cancer cell line SKOV3, measured by a modified Cell Titer Glo (CTG) assay. % INH is plotted against anti-CD73 antibody concentrations. The anti-CD73 antibodies tested are 350 and 373, expressed in either the .A or .B format.

FIGS. 9A and 9B are graphs similar to FIGS. 8A and 8B. The antibodies tested are the anti-CD73 antibodies 350, 356, 373, and 374, in the .A or .B format.

FIG. 10 is a graph showing inhibition of human CD73 over-expressed on HEK 293 cells by anti-CD73 antibodies, measured by a modified Cell Titer Glo (CTG) assay. % INH AMP conversion is plotted against a range of anti-CD73 antibody concentrations. The antibodies tested are the anti-CD73 antibodies 350, 356, 373, and 374, in the .A or .B format.

FIGS. 11A and 11B are graphs showing inhibition of CD73 expressed on primary human PBMCs isolated from two separate donors by anti-CD73 antibodies, measured by a modified Cell Titer Glo (CTG) assay. % INH is plotted against a range of anti-CD73 antibody concentrations. The anti-CD73 antibodies tested are 350, 356, and 358, all in the .B format.

FIGS. 12A and 12B are graphs showing the proliferation of anti-CD3/28 stimulated CD4+ T cells in the presence of AMP and an anti-CD73 antibody. Proliferation index, a measure of T cell division, is plotted against a range of antibody concentrations. FIG. 12A shows results from a study testing the anti-CD73 antibodies 350, 356, 358, 374, 377, and 379, all in the .B format. FIG. 12B shows results from a study using the antibodies 350 and 372, expressed in either the .A or .B format.

FIG. 13 is a bar graph showing the results of a xenograft study testing the ability of anti-CD73 antibody to inhibit the enzymatic activity of CD73 in vivo. The y-axis shows adenosine and inosine levels in the serum of immunocompromised mice implanted with a high-CD73 expressing breast cancer cell line (MDA-MB-231) as measured by mass spectrometry. The anti-CD73 antibodies 350, 356, 373, and 374, expressed in either the .A or .B format, were administered intraperitoneally at 20 or 200 μg/mouse. The control polyclonal human IgG antibody was administered at 200 μg/mouse.

FIG. 14 is a graph showing comparison of protection profiles of 373.A and 373.B, as measured by fragmentation hydrogen deuterium-exchange mass spectrometry (HDx-MS). FIG. 14 shows results for 1-min in-exchange at pH 7.5 and room temperature.

FIG. 15 is a graph showing comparison of protection profiles of 350.A2 and 350.B, as measured by fragmentation hydrogen deuterium-exchange mass spectrometry (HDx-MS). FIG. 15 shows results for 1-min in-exchange at pH 7.5 and room temperature.

FIG. 16A is a graph showing conformational change of CD73 between closed/active (Protein data bank (PDB) 4H21) and open/inactive conformation (PDB 4H2F). FIG. 16B is a graph showing open/open conformation of CD73 dimer constructed from two units of PDB 4H2F after alignment of c-terminal domains with PDB 4H1S.

FIGS. 17A and 17B are graphs showing SEC profiles of CD73-373.A and CD73-373.B complexes, respectively.

FIGS. 18A and 18B are graphs showing SEC profiles of CD73-350.A2 and CD73-350.B complexes, respectively.

FIG. 19 is a graph showing the relative percentages of CD73/mAb species calculated using the integration method.

FIG. 20 is a schematic graph showing an oligomerization model for unifying HDx and SEC interpretations.

FIGS. 21A and 21B are graphs showing expression of CD73 in stromal cell subsets analyzed by flow cytometry in 4T1 tumors from mice treated with a pan anti-TGFβ antibody or hIgG2 isotype control. In FIG. 21A, CD73 expression is displayed as fluorescence intensity, as compared to samples stained with a matched isotype control. One representative sample is shown. In FIG. 21B, quantification of CD73 expression, measured as mean fluorescence intensity, is depicted. Each symbol represents a sample. P-values are indicated where significant. CAF=cancer associated fibroblasts.

FIGS. 22A, 22B, and 22C are graphs showing results from an assay assessing the impact of an anti-CD73 antibody on class switch recombination in B cells. Naive (CD19⁺CD27⁻IgM⁺IgD⁺) CD73⁺ and CD73⁻ B cells were isolated from peripheral blood of healthy donors, labelled with CFSE and stimulated in vitro with CpG 2006, anti-CD40, IL-2, IL-21 and tranferrin. Cells were cultured in the presence of the anti-CD73 antibody 350.B or an isotype control antibody (IgG4) at three different concentrations: 1, 10 and 100 ng/mL. In FIG. 22A, IgM secretion was measured using an ELISA assay in the 7-day culture supernatant. In FIG. 22B, B cell proliferation was measured on day 7 and the numbers of divided cells counted after electronically gating on the CFSE diluted population are plotted for the three different concentrations tested. In FIG. 22C, IgG secreting cells (IgG SCs) were enumerated by an ELISPOT assay at the same time point. Data were collected from two independent experiments.

FIG. 23 is a panel of graphs showing individual tumor volumes for the indicated groups. Isotype control mice were euthanized on Day 25. In the anti-PD-1-treated group, mice received 300 μg/mouse for all doses. In the 350.B-treated group, mice received 600 μg/mouse for the first dose, followed by 400 μg/mouse for the four remaining doses. The same dosing schemes were administered for the combination group. All mice were treated on Days 2, 5, 9, 12 and 17.

FIG. 24A is a pair of graphs showing detection of biotinylated 373.A on unlabeled 373.A pretreated blood samples from two donors. FIG. 24B is a graph showing percent target occupancy on CD8+ T cells by the antibody 373.A.

FIGS. 25A and 25B are a panel of graphs showing that proliferation of CD4+ and CD8+ T cells was suppressed by AMP during TCR-mediated activation, and this suppression could be restored by the antibody 373.A.

### BRIEF DESCRIPTION OF THE TABLES

Table 1 provides amino acid and nucleotide sequences for exemplary anti-CD73 antibodies.

Tables 2, 26 and 27 provide consensus CDR sequences for exemplary anti-CD73 antibodies.

Table 3 provides amino acid sequences of human IgG heavy chains and human kappa light chain.

Table 4 provides exemplary sequences of CD73.

Tables 5 and 6 provide amino acid and/or nucleotide sequences of exemplary anti-PD-1 antibody molecules.

Tables 7 and 8 provide amino acid and/or nucleotide sequences of exemplary anti-PD-L1 antibody molecules.

Tables 9 and 10 provide amino acid and/or nucleotide sequences of exemplary anti-LAG-3 antibody molecules.

Tables 11 and 12 provide amino acid and/or nucleotide sequences of exemplary anti-TIM-3 antibody molecules.

Tables 13 and 14 provide amino acid and/or nucleotide sequences of exemplary anti-GITR antibody molecules.

Table 15 provides amino acid sequences of exemplary anti-CD3 bispecific antibody molecules.

Tables 16 and 17 provide amino acid sequences of exemplary IL15/IL-15Ra complexes.

Table 18 is a summary of selected therapeutic agents that can be administered in combination with the anti-CD73 antibody molecules described herein. Table 18 provides from left to right the following: the Compound Designation of the second therapeutic agent, the Compound structure, and Patent publication(s) disclosing the Compound.

Table 19 provides nomenclatures for two lineages of anti-CD73 antibodies.

Table 20 provides affinities of anti-CD73 antibodies.

Table 21 provides affinities of anti-CD73 Fabs.

Table 22 provides provisional dose levels for 373.A.

Table 23 provides provisional dose levels for 373.A in combination with PBF509.

Table 24 provides provisional dose levels for 373.A in combination with BAP049-Clone-E.

Table 25 provides provisional dose levels for PBF509 in combination with 373.A and BAP049-Clone-E.

Table 28 provides corresponding germline sequences of exemplary anti-CD73 antibodies.

### DETAILED DESCRIPTION

The term “CD73” as used herein refers to “Cluster of Differentiation 73,” also known as 5′-nucleotidase (5′-NT) or ecto-5′-nucleotidase. The term “CD73” includes mutants, fragments, variants, isoforms, and homologs of full-length wild-type CD73. In one embodiment, the protein CD73 is encoded by the NT5E gene. Exemplary CD73 sequences are available at the Uniprot database under accession numbers Q6NZX3 and P21589. Exemplary immature CD73 amino acid sequences are provided as SEQ ID NOs: 105-107. A “CD73 monomer” refers to a polypeptide comprising an extracellular domain of CD73. In one embodiment, a CD73 monomer is a full-length CD73. A “CD73 dimer” refers to two polypeptides (e.g., two non-covalently associated polypeptides) consisting of two CD73 monomers (e.g., two identical CD73 monomers) interacting with each other to form a stable dimer, e.g., a dimer formed via protein-protein interactions between the C-terminal domains of the CD73 monomers. In one embodiment, the CD73 dimer is a naturally-occurring CD73 dimer.

Without wishing to be bound by theory, human CD73 has two domains. A conserved N-terminal domain (corresponding to approximately residues 29-310 of SEQ ID NO: 105) and a conserved C-terminal domain (corresponding to approximately residues 343-513 of SEQ ID NO: 105), which are linked by a single a-helix (corresponding to approximately residues 318-336 of SEQ ID NO: 105). The active site is detected primarily in the closed conformation and is formed between C- and N-terminal domains. For enzyme catalysis, a domain motion of ˜100° of the N-terminal domain with respect to the C-terminal domain can enable substrate binding and release, which occurs in the open (catalytic inactive) conformation. Human CD73 forms a dimer through protein-protein interactions between C-terminal domains. The buried surface area as well as the molecular interactions at the dimer interface are significantly different between active and inactive conformations of the enzyme. See, e.g., Knapp K, et al., Structure 20:2161-73 (2012), incorporated herein by reference in its entirety.

Accordingly, the present invention provides, at least in part, antibody molecules that bind to CD73 with high affinity and specificity. In one embodiment, disclosed herein are human antibodies that bind to CD73. In one embodiment, disclosed herein are antibody molecules that are capable of inhibiting or reducing the enzymatic activity of CD73, e.g., human CD73, e.g., soluble human CD73 or membrane-bound human CD73. In one embodiment, disclosed herein are antibody molecules that are capable of inhibiting or reducing CD73-mediated conversion of adenosine monophosphate (AMP) to adenosine. Additional aspects of the invention include nucleic acid molecules encoding the antibody molecules, expression vectors, host cells and methods for making the antibody molecules. Immunoconjugates, multi- or bispecific molecules and pharmaceutical compositions comprising the antibody molecules are also provided. The anti-CD73 antibody molecules disclosed herein can be used to treat, prevent and/or diagnose cancerous or malignant disorders, e.g., solid and liquid tumors, e.g., lung cancer (e.g., non-small cell lung cancer), pancreas cancer (e.g., pancreatic ductal adenocarcinoma), breast cancer (e.g., triple-negative breast cancer), melanoma, head and neck cancer (e.g., squamous head and neck cancer), colorectal cancer (e.g., microsatellite stable (MSS) colorectal cancer), ovarian cancer, or renal cancer (e.g., renal cell carcinoma). The anti-CD73 antibody molecules disclosed herein can be used to treat, prevent and/or diagnose an infectious disease. Thus, methods for detecting CD73, as well as methods for treating various disorders, including cancer and infectious diseases, using the anti-CD73 antibody molecules are disclosed herein.

Additional terms are defined below and throughout the application.

As used herein, the articles “a” and “an” refer to one or to more than one (e.g., to at least one) of the grammatical object of the article. As used herein, “plurality” means two or more.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The compositions and methods disclosed herein encompass polypeptides and nucleic acids having the sequences specified, or sequences substantially identical or similar thereto, e.g., sequences having at least about 85%, 90%, or 95% sequence identity to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, e.g., at least 40%, 50%, 60%, e.g., at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a some embodiments, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In certain embodiments, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. One suitable set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid as described herein. BLAST protein searches can be performed with the XBLAST program, score=50, wordlength=3 to obtain amino acid sequences homologous to protein molecules described herein. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi nlm nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are suitable conditions and the ones that should be used unless otherwise specified.

It is understood that the molecules of the invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide,” “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a nonnatural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. In some embodiments, a CAR comprises at least an extracellular antigen binding domain, a transmembrane domain, and a cytoplasmic signaling domain (also referred to herein as “an intracellular signaling domain”) comprising a functional signaling domain derived from a stimulatory molecule and/or costimulatory molecule as defined below. In some aspects, the set of polypeptides are contiguous with each other. In some embodiments, the set of polypeptides include a dimerization switch that, upon the presence of a dimerization molecule, can couple the polypeptides to one another, e.g., can couple an antigen binding domain to an intracellular signaling domain In one aspect, the stimulatory molecule is the zeta chain associated with the T cell receptor complex. In one aspect, the cytoplasmic signaling domain further comprises one or more functional signaling domains derived from at least one costimulatory molecule as defined below. In one aspect, the costimulatory molecule is chosen from the costimulatory molecules described herein, e.g., 4-1BB (i.e., CD137), CD27 and/or CD28. In one aspect, the CAR comprises a chimeric fusion protein comprising an extracellular antigen binding domain, a transmembrane domain, and an intracellular signaling domain comprising a functional signaling domain derived from a stimulatory molecule. In one aspect, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In one aspect, the CAR comprises a chimeric fusion protein comprising an extracellular antigen binding domain, a transmembrane domain, and an intracellular signaling domain comprising two functional signaling domains derived from one or more costimulatory molecule(s) and a functional signaling domain derived from a stimulatory molecule. In one aspect, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

The term “signaling domain” refers to the functional portion of a protein which acts by transmitting information within the cell to regulate cellular activity via defined signaling pathways by generating second messengers or functioning as effectors by responding to such messengers.

An “intracellular signaling domain,” as the term is used herein, refers to an intracellular portion of a molecule. The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and DAP12.

The term “zeta” or alternatively “zeta chain”, “CD3-zeta” or “TCR-zeta” is defined as the protein provided as GenBank Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain, or functional derivatives thereof, that are sufficient to functionally transmit an initial signal necessary for T cell activation. In one aspect the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof.

The term a “costimulatory molecule” refers to a cognate binding partner on a T cell that binds to a costimulatory ligand, thereby mediating a costimulatory response by the T cell, such as, but not limited to, proliferation. Costimulatory molecules are cell surface molecules other than antigen receptors or their ligands that are contribute to an efficient immune response. Costimulatory molecules include, but are not limited to an MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that binds to CD83.

A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, ICAM-1, lymphocyte function-associated antigen-1 (LFA-1), CD2, CDS, CD7, CD287, LIGHT, NKG2C, NKG2D, SLAMF7, NKp80, NKp30, NKp44, NKp46, CD160, B7-H3, and a ligand that binds to CD83, and the like.

The intracellular signaling domain can comprise the entire intracellular portion, or the entire native intracellular signaling domain, of the molecule from which it is derived, or a functional fragment or derivative thereof.

The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a non-human species, e.g., mouse, rodent, monkey, ape and the like.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloid-derived phagocytes.

“Immune effector function” or “immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs of the present invention includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

Various aspects of the compositions and methods herein are described in further detail below. Additional definitions are set out throughout the specification.

### Antibody Molecules

In one embodiment, the antibody molecule binds to a mammalian, e.g., human, CD73. For example, the antibody molecule binds to an epitope, e.g., linear or conformational epitope, e.g., an epitope as described herein, on CD73.

As used herein, the term “antibody molecule” refers to a protein, e.g., an immunoglobulin chain or fragment thereof, comprising at least one immunoglobulin variable domain sequence. The term “antibody molecule” includes, for example, a monoclonal antibody (including a full length antibody which has an immunoglobulin Fc region). In an embodiment, an antibody molecule comprises a full length antibody, or a full length immunoglobulin chain. In an embodiment, an antibody molecule comprises an antigen binding or functional fragment of a full length antibody, or a full length immunoglobulin chain.

As used herein, an antibody molecule “binds to” an antigen as such binding is understood by one skilled in the art. In one embodiment, an antibody binds to an antigen with a dissociation constant (K_{D}) of about 1×10³ M or less, 1×10⁴M or less, or 1×10⁻⁵ M or less.

In an embodiment, an antibody molecule is a monospecific antibody molecule and binds a single epitope, e.g., a monospecific antibody molecule having a plurality of immunoglobulin variable domain sequences, each of which binds the same epitope.

In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein).

In an embodiment, the first and second epitopes overlap or substantially overlap. In an embodiment, the first and second epitopes do not overlap or do not substantially overlap. In an embodiment, the first and second epitopes are on different antigens, e.g., different proteins (or different subunits of a multimeric protein). In an embodiment, a multispecific antibody molecule comprises a third, fourth or fifth immunoglobulin variable domain In an embodiment, a multispecific antibody molecule is a bispecific antibody molecule, a trispecific antibody molecule, or tetraspecific antibody molecule.

In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., different proteins (or different subunits of a multimeric protein). In an embodiment, a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody molecule comprises a half antibody having binding specificity for a first epitope and a half antibody having binding specificity for a second epitope. In an embodiment, a bispecific antibody molecule comprises a half antibody, or fragment thereof, having binding specificity for a first epitope and a half antibody, or fragment thereof, having binding specificity for a second epitope. In an embodiment, a bispecific antibody molecule comprises a scFv, or fragment thereof, have binding specificity for a first epitope and a scFv, or fragment thereof, have binding specificity for a second epitope.

In an embodiment, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable domain sequence (abbreviated herein as VH), and a light (L) chain variable domain sequence (abbreviated herein as VL). In an embodiment, an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. A preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments may be obtained using any suitable method, including conventional techniques known to those with skill in the art, and the fragments can be screened for utility in the same manner as are intact antibodies.

The term “antibody” includes intact molecules as well as functional fragments thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

The antibodies disclosed herein can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region” and “CDR” as used herein refer to the sequences of amino acids within antibody variable regions which confer antigen specificity and binding affinity. In some embodiments, there are three CDRs in each heavy chain variable region (HCDR1, HCDR2, and HCDR3) and three CDRs in each light chain variable region (LCDR1, LCDR2, and LCDR3).

The precise amino acid sequence boundaries of a given CDR can be determined using any of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme). As used herein, the CDRs defined according to the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

Under all definitions, each VH and VL typically includes three CDRs and four FRs, arranged from amino-terminus to carboxy-terminus in the following order: FR1, CDR1, FR2, CDR2, FR3, CDR3, FR4.

Generally, unless specifically indicated, the anti-CD73 antibody molecules can include any combination of one or more Kabat CDRs, Chothia CDRs, combination of Kabat and Chothia CDRs, IMGT CDRs, and/or an alternative definition, e.g., described in Table 1.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an antibody molecule that comprises determinants that form an interface that binds to a CD73 polypeptide, or an epitope thereof. With respect to proteins (or protein mimetics), the antigen-binding site typically includes one or more loops (of at least, e.g., four amino acids or amino acid mimics) that form an interface that binds to a CD73 polypeptide. Typically, the antigen-binding site of an antibody molecule includes at least one or two CDRs and/or hypervariable loops, or more typically at least three, four, five or six CDRs and/or hypervariable loops.

As used herein, the term “Eu numbering” refers to the Eu numbering convention for the constant regions of an antibody, as described in Edelman, G. M. et al., Proc. Natl. Acad. USA, 63, 78-85 (1969) and Kabat et al., in “Sequences of Proteins of Immunological Interest”, U.S. Dept. Health and Human Services, 5th edition, 1991.

The terms “compete” or “cross-compete” are used interchangeably herein to refer to the ability of an antibody molecule to interfere with binding of an anti-CD73 antibody molecule, e.g., an anti-CD73 antibody molecule provided herein, to a target, e.g., human CD73. The interference with binding can be direct or indirect (e.g., through an allosteric modulation of the antibody molecule or the target). The extent to which an antibody molecule is able to interfere with the binding of another antibody molecule to the target, and therefore whether it can be said to compete, can be determined using a competition binding assay, for example, a flow cytometry assay, an ELISA or BIACORE assay. In some embodiments, a competition binding assay is a quantitative competition assay. In some embodiments, a first anti-CD73 antibody molecule is said to compete for binding to the target with a second anti-CD73 antibody molecule when the binding of the first antibody molecule to the target is reduced by 10% or more, e.g., 20% or more, 30% or more, 40% or more, 50% or more, 55% or more, 60% or more, 65% or more, 70% or more, 75% or more, 80% or more, 85% or more, 90% or more, 95% or more, 98% or more, 99% or more in a competition binding assay (e.g., a competition assay described herein).

As used herein, the term “epitope” refers to the moieties of an antigen (e.g., human CD73) that specifically interact with an antibody molecule. Such moieties, also referred to herein as epitopic determinants, typically comprise, or are part of, elements such as amino acid side chains or sugar side chains. An epitopic determinant can be defined by methods known in the art or disclosed herein, e.g., by crystallography or by hydrogen-deuterium exchange. At least one or some of the moieties on the antibody molecule that specifically interact with an epitopic determinant are typically located in a CDR(s). Typically, an epitope has a specific three dimensional structural characteristics. Typically, an epitope has specific charge characteristics. Some epitopes are linear epitopes while others are conformational epitopes.

In an embodiment, an epitopic determinant is a moiety on the antigen, e.g., such as amino acid side chain or sugar side chain, or part thereof, which, when the antigen and antibody molecule are co-crystallized, is within a predetermined distance, e.g., within 5 Angstroms, of a moiety on the antibody molecule, referred to herein as a “crystallographic epitopic determinant.” The crystallographic epitopic determinants of an epitope are collectively referred to as the “crystallographic epitope.”

A first antibody molecule binds the same epitope as a second antibody molecule (e.g., a reference antibody molecule, e.g., an antibody molecule disclosed herein) if the first antibody interacts with the same epitopic determinants on the antigen as does the second or reference antibody, e.g., when interaction is measured in the same way for both the antibody and the second or reference antibody. Epitopes that overlap share at least one epitopic determinant. A first antibody molecule binds an overlapping epitope with a second antibody molecule (e.g., a reference antibody molecule, e.g., an antibody disclosed herein) when both antibody molecules interact with a common epitopic determinant. A first and a second antibody molecule (e.g., a reference antibody molecule, e.g., an antibody molecule disclosed herein) bind substantially overlapping epitopes if at least half of the epitopic determinants of the second or reference antibody are found as epitopic determinants in the epitope of the first antibody. A first and a second antibody molecule (e.g., a reference antibody molecule, e.g., an antibody molecule disclosed herein) bind substantially the same epitope if the first antibody molecule binds at least half of the core epitopic determinants of the epitope of the second or reference antibody, wherein the core epitopic determinants are defined by, e.g., crystallography or hydrogen-deuterium exchange.

As used herein, an antibody molecule “reduces hydrogen-deuterium exchange” in an antigen fragment when the hydrogen-deuterium exchange in the antigen fragment in the presence of the antibody molecule is lower than the hydrogen-deuterium exchange in the antigen fragment in the absence of the antibody molecule, as measured in a hydrogen-deuterium exchange assay.

As used herein, a reduction in “the average hydrogen-deuterium exchange” is determined by the level of normalized hydrogen-deuterium exchange (Da per residue) in an antigen fragment in the absence of an antibody minus the level of normalized hydrogen-deuterium exchange (Da per residue) in the antigen fragment in the presence of the antibody.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

The antibody molecule can be a polyclonal or a monoclonal antibody. In other embodiments, the antibody can be recombinantly produced, e.g., produced by yeast display, phage display, or by combinatorial methods. Alternatively, such antibodies may be selected from synthetic yeast-based antibody presentation systems, such as those described in, e.g., Y. Xu et at, Addressing polyspecificity of antibodies selected from an in vitro yeast presentation system: a FACS-based, high-throughput selection and analytical tool. PEDS 26.10. 663-70 ((2013); WO2009036379; WO2010105256; and WO2012009568, herein incorporated by reference in their entireties.

In one embodiment, the antibody is a fully human antibody (e.g., an antibody produced by yeast display, an antibody produced by phage display, or an antibody made in a mouse which has been genetically engineered to produce an antibody from a human immunoglobulin sequence), or a non-human antibody, e.g., a rodent (mouse or rat), goat, primate (e.g., monkey), or camel antibody. Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Antibodies can be produced by any suitable recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immunoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to CD73. In some embodiments, the donor is a rodent antibody, e.g., a rat or mouse antibody, and the recipient is a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, e.g., 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference.

Also within the scope of the invention are humanized antibodies in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFV) may be engineered (see, for example, Colcher, D. et al. (1999) Ann NY Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In some embodiments the antibody has effector function and can fix complement. In other embodiments the antibody does not recruit effector cells or fix complement. In certain embodiments, the antibody has reduced or no ability to bind an Fc receptor. For example, it may be an isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference) Amino acid mutations which stabilize antibody structure, such as S228P (Eu numbering) in human IgG4, are also contemplated. Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody molecule of the invention may be derivatized (or labeled) to include fluorescent compounds, various enzymes, prosthetic groups, luminescent materials, bioluminescent materials, fluorescent emitting metal atoms, e.g., europium (Eu), and other anthanides, and radioactive materials (described below). Exemplary fluorescent detectable agents include fluorescein, fluorescein isothiocyanate, rhodamine, 5dimethylamine-1-napthalenesulfonyl chloride, phycoerythrin and the like. An antibody may also be derivatized with detectable enzymes, such as alkaline phosphatase, horseradish peroxidase, β-galactosidase, acetylcholinesterase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody molecule may also be derivatized with a prosthetic group (e.g., streptavidin/biotin and avidin/biotin). For example, an antibody may be derivatized with biotin, and detected through indirect measurement of avidin or streptavidin binding.

Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of bioluminescent materials include luciferase, luciferin, and aequorin.

Labeled antibody molecule can be used, for example, diagnostically and/or experimentally in a number of contexts, including (i) to isolate a predetermined antigen by standard techniques, such as affinity chromatography or immunoprecipitation; (ii) to detect a predetermined antigen (e.g., in a cellular lysate or cell supernatant) in order to evaluate the abundance and pattern of expression of the protein; (iii) to monitor protein levels in tissue as part of a clinical testing procedure, e.g., to determine the efficacy of a given treatment regimen.

An antibody molecule may be conjugated to another molecular entity, typically a label or a therapeutic (e.g., immunomodulatory, immunostimularoty, cytotoxic, or cytostatic) agent or moiety. Radioactive isotopes can be used in diagnostic or therapeutic applications. Radioactive isotopes that can be coupled to the anti-CD73 antibodies include, but are not limited to α-, β-, or γ-emitters, or β- and γ-emitters. Such radioactive isotopes include, but are not limited to iodine (¹³¹I or ¹²⁵I), yttrium (⁹⁰Y), lutetium (¹⁷⁷Lu), actinium (²²⁵Ac), praseodymium, astatine (²¹¹At) rhenium (¹⁸⁶Re), bismuth (²¹²B1 or ²¹³Bi), indium (¹¹¹In) technetium (⁹⁹ mTc), phosphorus (³²P), rhodium (¹⁸⁸Rh), sulfur (³⁵S), carbon (¹⁴C), tritium (³H), chromium (⁵¹Cr), chlorine (³⁶C1), cobalt (⁵⁷Co or ⁵⁸Co), iron (⁵⁹Fe), selenium (⁷⁵Se), or gallium (⁶⁷Ga). Radioisotopes useful as therapeutic agents include yttrium (⁹⁰Y), lutetium (¹⁷⁷Lu), actinium (₂₂₅Ac), praseodymium, astatine (²¹¹At), rhenium (¹⁸⁶Re), bismuth (²¹²Bi or ²¹³Bi), and rhodium (¹⁸⁸Rh), Radioisotopes useful as labels, e.g., for use in diagnostics, include iodine (¹³¹I or ¹²⁵I), indium (¹¹¹In), technetium (^{99m}Tc), phosphorus (³²P), carbon (¹⁴C), and tritium (³H), or one or more of the therapeutic isotopes listed above.

The invention provides radiolabeled antibody molecules and methods of labeling the same. In one embodiment, a method of labeling an antibody molecule is disclosed. The method includes contacting an antibody molecule, with a chelating agent, to thereby produce a conjugated antibody. The conjugated antibody is radiolabeled with a radioisotope, e.g., ¹¹¹Indium, ⁹⁰Yttrium and ¹⁷⁷Lutetium, to thereby produce a labeled antibody molecule.

As is discussed above, the antibody molecule can be conjugated to a therapeutic agent. Therapeutically active radioisotopes have already been mentioned. Examples of other therapeutic agents include taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846, 545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclinies (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

In one aspect, the invention features a method of providing a target binding molecule that binds to a CD73 receptor. For example, the target binding molecule is an antibody molecule. The method includes: providing a target protein that comprises at least a portion of non-human protein, the portion being homologous to (at least 70, 75, 80, 85, 87, 90, 92, 94, 95, 96, 97, 98, or 99% identical to) a corresponding portion of a human target protein, but differing by at least one amino acid (e.g., at least one, two, three, four, five, six, seven, eight, or nine amino acids); obtaining an antibody molecule that binds to the antigen; and evaluating efficacy of the binding agent in modulating activity of the target protein. The method can further include administering the binding agent (e.g., antibody molecule) or a derivative (e.g., a humanized antibody molecule) to a human subject.

In certain embodiments, the antibody molecule is a multi-specific (e.g., a bispecific or a trispecific) antibody molecule. Protocols for generating bispecific or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhdryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U55637481; multivalent and multispecific binding proteins, e.g., dimer of polypeptides having first domain with binding region of Ig heavy chain variable region, and second domain with binding region of Ig light chain variable region, generally termed diabodies (higher order structures are also disclosed creating bispecifc, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., US5869620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002004587A1, US2002076406A1, US2002103345A1, US2003207346A1, US2003211078A1, US2004219643A1, US2004220388A1, US2004242847A1, US2005003403A1, US2005004352A1, US2005069552A1, US2005079170A1, US2005100543A1, US2005136049A1, US2005136051A1, US2005163782A1, US2005266425A1, US2006083747A1, US2006120960A1, US2006204493A1, US2006263367A1, US2007004909A1, US2007087381A1, US2007128150A1, US2007141049A1, US2007154901A1, US2007274985A1, US2008050370A1, US2008069820A1, US2008152645A1, US2008171855A1, US2008241884A1, US2008254512A1, US2008260738A1, US2009130106A1, US2009148905A1, US2009155275A1, US2009162359A1, US2009162360A1, US2009175851A1, US2009175867A1, US2009232811A1, US2009234105A1, US2009263392A1, US2009274649A1, EP346087A2, WO0006605A2, WO02072635A2, WO04081051A1, WO06020258A2, WO2007044887A2, WO2007095338A2, WO2007137760A2, WO2008119353A1, WO2009021754A2, WO2009068630A1, WO9103493A1, WO9323537A1, WO9409131A1, WO9412625A2, WO9509917A1, WO9637621A2, WO9964460A1. The contents of the above-referenced applications are incorporated herein by reference in their entireties.

In other embodiments, the anti-CD73 antibody molecule (e.g., a monospecific, bispecific, or multispecific antibody molecule) is covalently linked, e.g., fused, to another partner e.g., a protein e.g., one, two or more cytokines, e.g., as a fusion molecule for example a fusion protein.

A “fusion protein” and a “fusion polypeptide” refer to a polypeptide having at least two portions covalently linked together, where each of the portions is a polypeptide having a different property. The property may be a biological property, such as activity in vitro or in vivo. The property can also be simple chemical or physical property, such as binding to a target molecule, catalysis of a reaction, etc. The two portions can be linked directly by a single peptide bond or through a peptide linker, but are in reading frame with each other.

This invention provides an isolated nucleic acid molecule encoding the above antibody molecule, vectors and host cells thereof. The nucleic acid molecule includes but is not limited to RNA, genomic DNA and cDNA.

### Exemplary Anti-CD73 Antibody Molecules

In some embodiments, the anti-CD73 antibody molecule comprises at least one antigen-binding region, e.g., a variable region or an antigen-binding fragment thereof, from an antibody described herein, e.g., an antibody chosen from 918, 350, 356, 358, 930, 373, 374, 376, 377, or 379; or as described in Table 1; or encoded by a nucleotide sequence in Table 1; or a sequence substantially identical (e.g., having at least about 80%, 85%, 90%, 92%, 95%, 97%, 98%, or 99% sequence identity) to any of the aforesaid sequences.

In certain embodiments, the anti-CD73 antibody molecule comprises at least one, two, three, or four variable regions from an antibody described herein, e.g., an antibody chosen from 918, 350, 356, 358, 930, 373, 374, 376, 377, or 379; or as described in Table 1; or encoded by a nucleotide sequence in Table 1; or a sequence substantially identical (e.g., having at least about 80%, 85%, 90%, 92%, 95%, 97%, 98%, or 99% sequence identity) to any of the aforesaid sequences.

In some embodiments, the anti-CD73 antibody molecule comprises at least one or two heavy chain variable regions from an antibody described herein, e.g., an antibody chosen from 918, 350, 356, 358, 930, 373, 374, 376, 377, or 379; or as described in Table 1; or encoded by a nucleotide sequence in Table 1; or a sequence substantially identical (e.g., having at least about 80%, 85%, 90%, 92%, 95%, 97%, 98%, or 99% sequence identity) to any of the aforesaid sequences.

In certain embodiments, the anti-CD73 antibody molecule comprises at least one or two light chain variable regions from an antibody described herein, e.g., an antibody chosen from 918, 350, 356, 358, 930, 373, 374, 376, 377, or 379; or as described in Table 1; or encoded by a nucleotide sequence in Table 1; or a sequence substantially identical (e.g., having at least about 80%, 85%, 90%, 92%, 95%, 97%, 98%, or 99% sequence identity) to any of the aforesaid sequences.

In one embodiment, the anti-CD73 antibody molecule includes a heavy chain constant region of an IgG4, e.g., a human IgG4. In another embodiment, the human IgG4 includes a substitution (e.g., a Ser to Pro substitution) at position 228 according to Eu numbering. In still another embodiment, the anti-CD73 antibody molecule includes a heavy chain constant region of an IgG1, e.g., a human IgG1. In one embodiment, the human IgG1 includes a substitution (e.g., an Asn to Ala substitution) at position 297 according to Eu numbering. In one embodiment, the human IgG1 includes a substitution (e.g., an Asp to Ala substitution) at position 265 according to Eu numbering, a substitution (e.g., a Pro to Ala substitution) at position 329 according to Eu numbering, or both. In one embodiment, the human IgG1 includes a substitution (e.g., a Leu to Ala substitution) at position 234 according to Eu numbering, a substitution (e.g., a Leu to Ala substitution) at position 235 according to Eu numbering, or both. In one embodiment, the heavy chain constant region comprises an amino acid sequence set forth in Table 3, or a sequence substantially identical (e.g., having at least about 80%, 85%, 90%, 92%, 95%, 97%, 98%, or 99% sequence identity) thereto.

In yet another embodiment, the anti-CD73 antibody molecule includes a kappa light chain constant region, e.g., a human kappa light chain constant region. In one embodiment, the light chain constant region comprises an amino acid sequence set forth in Table 3, or a sequence substantially identical (e.g., having at least about 80%, 85%, 90%, 92%, 95%, 97%, 98%, or 99% sequence identity) thereto.

In another embodiment, the anti-CD73 antibody molecule includes a heavy chain constant region of an IgG4, e.g., a human IgG4, and a kappa light chain constant region, e.g., a human kappa light chain constant region, e.g., a heavy and light chain constant region comprising an amino acid sequence set forth in Table 3, or a sequence substantially identical (e.g., having at least about 80%, 85%, 90%, 92%, 95%, 97%, 98%, or 99% sequence identity) thereto. In yet another embodiment, the anti-CD73 antibody molecule includes a heavy chain constant region of an IgG1, e.g., a human IgG1, and a kappa light chain constant region, e.g., a human kappa light chain constant region, e.g., a heavy and light chain constant region comprising an amino acid sequence set forth in Table 3, or a sequence substantially identical (e.g., having at least about 80%, 85%, 90%, 92%, 95%, 97%, 98%, or 99% sequence identity) thereto. In one embodiment, the human IgG1 includes a substitution at position 297 according to Eu numbering (e.g., an Asn to Ala substitution). In one embodiment, the human IgG1 includes a substitution at position 265 according to Eu numbering, a substitution at position 329 according to Eu numbering, or both (e.g., an Asp to Ala substitution at position 265 and/or a Pro to Ala substitution at position 329). In one embodiment, the human IgG1 includes a substitution at position 234 according to Eu numbering, a substitution at position 235 according to Eu numbering, or both (e.g., a Leu to Ala substitution at position 234 and/or a Leu to Ala substitution at position 235).

In another embodiment, the anti-CD73 antibody molecule includes a heavy chain variable region and a constant region, a light chain variable region and a constant region, or both, comprising the amino acid sequence of 918, 350, 356, 358, 930, 373, 374, 376, 377, or 379; or as described in Table 1; or encoded by a nucleotide sequence in Table 1; or a sequence substantially identical (e.g., having at least about 80%, 85%, 90%, 92%, 95%, 97%, 98%, or 99% sequence identity) to any of the aforesaid sequences.

In some embodiments, the anti-CD73 antibody molecule includes at least one, two, or three complementarity determining regions (CDRs) from a heavy chain variable region of an antibody described herein, e.g., an antibody chosen from 918, 350, 356, 358, 930, 373, 374, 376, 377, or 379; or as described in Table 1, or encoded by a nucleotide sequence in Table 1; or a sequence substantially identical (e.g., having at least about 80%, 85%, 90%, 92%, 95%, 97%, 98%, or 99% sequence identity) to any of the aforesaid sequences.

In some embodiments, the anti-CD73 antibody molecule comprises at least one, two, or three complementarity determining regions (CDRs) from a heavy chain variable region comprising an amino acid sequence shown in Table 1, or encoded by a nucleotide sequence in Table 1. In one embodiment, one or more of the CDRs (or collectively all of the CDRs) have one, two, three, four, five or more changes, e g., amino acid substitutions, insertions, or deletions, relative to the amino acid sequence shown in Table 1, or encoded by a nucleotide sequence shown in Table 1. In certain embodiments, the anti-CD73 antibody molecule includes a substitution in a heavy chain CDR, e.g., one or more substitutions in a CDR1, CDR2 and/or CDR3 of the heavy chain.

In some embodiments, the anti-CD73 antibody molecule includes at least one, two, or three complementarity determining regions (CDRs) from a light chain variable region of an antibody described herein, e.g., an antibody chosen from 918, 350, 356, 358, 930, 373, 374, 376, 377, or 379; or as described in Table 1, or encoded by a nucleotide sequence in Table 1; or a sequence substantially identical (e.g., having at least about 80%, 85%, 90%, 92%, 95%, 97%, 98%, or 99% sequence identity) to any of the aforesaid sequences.

In certain embodiments, the anti-CD73 antibody molecule includes at least one, two, or three CDRs (or collectively all of the CDRs) from a light chain variable region comprising an amino acid sequence shown in Table 1, or encoded by a nucleotide sequence shown in Table 1. In some embodiments, one or more of the CDRs (or collectively all of the CDRs) have one, two, three, four, five, six or more changes, e.g., amino acid substitutions, insertions, or deletions, relative to the CDRs shown in Table 1, or encoded by a nucleotide sequence shown in Table 1. In some embodiments, the anti-CD73 antibody molecule includes at least one, two, or three CDRs (or collectively all of the CDRs) from a light chain variable region comprising an amino acid sequence shown in Table 1, or encoded by a nucleotide sequence shown in Table 1. In some embodiments, one or more of the CDRs (or collectively all of the CDRs) have one, two, three, four, five, six or more changes, e g, amino acid substitutions, insertions, or deletions, relative to the CDRs shown in Table 1, or encoded by a nucleotide sequence shown in Table 1.

In some embodiments, the anti-CD73 antibody molecule includes at least one, two, three, four, five or six CDRs (or collectively all of the CDRs) from a heavy and light chain variable region comprising an amino acid sequence shown in Table 1, or encoded by a nucleotide sequence shown in Table 1. In some embodiments, one or more of the CDRs (or collectively all of the CDRs) have one, two, three, four, five, six or more changes, e.g., amino acid substitutions, insertions, or deletions, relative to the CDRs shown in Table 1, or encoded by a nucleotide sequence shown in Table 1.

In certain embodiments, the anti-CD73 antibody molecule includes all six CDRs from an antibody described herein, e.g., an antibody chosen from 918, 350, 356, 358, 930, 373, 374, 376, 377, or 379; or as described in Table 1; or encoded by a nucleotide sequence in Table 1, or closely related CDRs, e.g., CDRs which are identical or which have at least one amino acid alteration, but not more than two, three or four alterations (e.g., substitutions, deletions, or insertions, e.g., conservative substitutions). In certain embodiments, the anti-CD73 antibody molecule may include any CDR described herein. In certain embodiments, the anti-CD73 antibody molecule includes a substitution in a heavy chain CDR, e.g., one or more substitutions in a CDR1, CDR2 and/or CDR3 of the heavy chain.

In some embodiments, the anti-CD73 antibody molecule includes at least one, two, or three CDRs according to Kabat et al. (e.g., at least one, two, or three CDRs according to the Kabat definition as set out in Table 1) from a heavy chain variable region of an antibody described herein, e.g., an antibody chosen from 918, 350, 356, 358, 930, 373, 374, 376, 377, or 379; or as described in Table 1; or encoded by a nucleotide sequence in Table 1; or a sequence substantially identical (e.g., having at least about 80%, 85%, 90%, 92%, 95%, 97%, 98%, or 99% sequence identity) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 1.

In certain embodiments, the anti-CD73 antibody molecule includes at least one, two, or three CDRs according to Kabat et al. (e.g., at least one, two, or three CDRs according to the Kabat definition as set out in Table 1) from a light chain variable region of an antibody described herein, e.g., an antibody chosen from 918, 350, 356, 358, 930, 373, 374, 376, 377, or 379; or encoded by a nucleotide sequence in Table 1; or a sequence substantially identical (e.g., having at least about 80%, 85%, 90%, 92%, 95%, 97%, 98%, or 99% sequence identity) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 1.

In certain embodiments, the anti-CD73 antibody molecule includes at least one, two, three, four, five, or six CDRs according to Kabat et al. (e.g., at least one, two, three, four, five, or six CDRs according to the Kabat definition as set out in Table 1) from the heavy and light chain variable regions of an antibody described herein, e.g., an antibody chosen from 918, 350, 356, 358, 930, 373, 374, 376, 377, or 379; or as described in Table 1; or encoded by a nucleotide sequence in Table 1; or a sequence substantially identical (e.g., having at least about 80%, 85%, 90%, 92%, 95%, 97%, 98%, or 99% sequence identity) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 1.

In some embodiments, the anti-CD73 antibody molecule includes all six CDRs according to Kabat et al. (e.g., all six CDRs according to the Kabat definition as set out in Table 1) from the heavy and light chain variable regions of an antibody described herein, e.g., an antibody chosen from 918, 350, 356, 358, 930, 373, 374, 376, 377, or 379; or as described in Table 1; or encoded by a nucleotide sequence in Table 1; or a sequence substantially identical (e.g., having at least about 80%, 85%, 90%, 92%, 95%, 97%, 98%, or 99% sequence identity) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 1. In one embodiment, the anti-CD73 antibody molecule may include any CDR described herein.

In some embodiments, the anti-CD73 antibody molecule includes at least one, two, or three Chothia hypervariable loops (e.g., at least one, two, or three hypervariable loops according to the Chothia definition as set out in Table 1) from a heavy chain variable region of an antibody described herein, e.g., an antibody chosen from 918, 350, 356, 358, 930, 373, 374, 376, 377, or 379; or as described in Table 1; or encoded by a nucleotide sequence in Table 1; or at least the amino acids from those hypervariable loops that contact CD73; or which have at least one amino acid alteration, but not more than two, three or four alterations (e.g., substitutions, deletions, or insertions, e.g., conservative substitutions) relative to one, two, or three hypervariable loops according to Chothia et al. shown in Table 1.

In certain embodiments, the anti-CD73 antibody molecule includes at least one, two, or three Chothia hypervariable loops (e.g., at least one, two, or three hypervariable loops according to the Chothia definition as set out in Table 1) of a light chain variable region of an antibody described herein, e.g., an antibody chosen from 918, 350, 356, 358, 930, 373, 374, 376, 377, or 379; or as described in Table 1; or encoded by a nucleotide sequence in Table 1; or at least the amino acids from those hypervariable loops that contact CD73; or which have at least one amino acid alteration, but not more than two, three or four alterations (e.g., substitutions, deletions, or insertions, e.g., conservative substitutions) relative to one, two, or three hypervariable loops according to Chothia et al. shown in Table 1.

In certain embodiments, the anti-CD73 antibody molecule includes at least one, two, three, four, five, or six hypervariable loops (e.g., at least one, two, three, four, five, or six hypervariable loops according to the Chothia definition as set out in Table 1) from the heavy and light chain variable regions of an antibody described herein, e.g., an antibody chosen from 918, 350, 356, 358, 930, 373, 374, 376, 377, or 379; or as described in Table 1; or encoded by a nucleotide sequence in Table 1; or at least the amino acids from those hypervariable loops that contact CD73; or which have at least one amino acid alteration, but not more than two, three or four alterations (e.g., substitutions, deletions, or insertions, e.g., conservative substitutions) relative to one, two, three, four, five or six hypervariable loops according to Chothia et al. shown in Table 1.

In some embodiments, the anti-CD73 antibody molecule includes all six hypervariable loops (e.g., all six hypervariable loops according to the Chothia definition as set out in Table 1) of an antibody described herein, e.g., an antibody chosen from 918, 350, 356, 358, 930, 373, 374, 376, 377, or 379; or closely related hypervariable loops, e.g., hypervariable loops which are identical or which have at least one amino acid alteration, but not more than two, three or four alterations (e.g., substitutions, deletions, or insertions, e.g., conservative substitutions); or which have at least one amino acid alteration, but not more than two, three or four alterations (e.g., substitutions, deletions, or insertions, e.g., conservative substitutions) relative to all six hypervariable loops according to Chothia et al. shown in Table 1. In one embodiment, the anti-CD73 antibody molecule may include any hypervariable loop described herein. In still another embodiment, the anti-CD73 antibody molecule includes at least one, two, or three hypervariable loops that have the same canonical structures as the corresponding hypervariable loop of an antibody described herein, e.g., an antibody chosen from 918, 350, 356, 358, 930, 373, 374, 376, 377, or 379, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In certain embodiments, the anti-CD73 antibody molecule includes a combination of CDRs or hypervariable loops defined according to the Kabat et al. and Chothia et al.

In one embodiment, the anti-CD73 antibody molecule includes at least one, two or three CDRs or hypervariable loops from a heavy chain variable region of an antibody described herein, e.g., an antibody chosen from 918, 350, 356, 358, 930, 373, 374, 376, 377, or 379, according to the Kabat and Chothia definition (e.g., at least one, two, or three CDRs or hypervariable loops according to the Kabat and Chothia definition as set out in Table 1); or encoded by a nucleotide sequence in Table 1; or a sequence substantially identical (e.g., having at least about 80%, 85%, 90%, 92%, 95%, 97%, 98%, or 99% sequence identity) to any of the aforesaid sequences; or which have at least one amino acid alteration, but not more than two, three or four alterations (e.g., substitutions, deletions, or insertions, e.g., conservative substitutions) relative to one, two, or three CDRs or hypervariable loops according to Kabat and/or Chothia shown in Table 1.

In another embodiment, the anti-CD73 antibody molecule includes at least one, two or three CDRs or hypervariable loops from a light chain variable region of an antibody described herein, e.g., an antibody chosen from 918, 350, 356, 358, 930, 373, 374, 376, 377, or 379, according to the Kabat and Chothia definition (e.g., at least one, two, or three CDRs or hypervariable loops according to the Kabat and Chothia definition as set out in Table 1); or encoded by a nucleotide sequence in Table 1; or a sequence substantially identical (e.g., having at least about 80%, 85%, 90%, 92%, 95%, 97%, 98%, or 99% sequence identity) to any of the aforesaid sequences; or which have at least one amino acid alteration, but not more than two, three or four alterations (e.g., substitutions, deletions, or insertions, e.g., conservative substitutions) relative to one, two, or three CDRs or hypervariable loops according to Kabat and/or Chothia shown in Table 1.

In some embodiments, the anti-CD73 antibody molecule includes at least one, two, or three CDRs according to the IMGT definition (e.g., at least one, two, or three CDRs according to the IMGT definition as set out in Table 1) from a heavy chain variable region of an antibody described herein, e.g., an antibody chosen from 918, 350, 356, 358, 930, 373, 374, 376, 377, or 379; or as described in Table 1; or encoded by a nucleotide sequence in Table 1; or a sequence substantially identical (e.g., having at least about 80%, 85%, 90%, 92%, 95%, 97%, 98%, or 99% sequence identity) to any of the aforesaid sequences; or which have at least one amino acid alteration, but not more than two, three or four alterations (e.g., substitutions, deletions, or insertions, e.g., conservative substitutions) relative to one, two, or three CDRs according to the IMGT definition shown in Table 1.

In certain embodiments, the anti-CD73 antibody molecule includes at least one, two, or three CDRs according to the IMGT definition (e.g., at least one, two, or three CDRs according to the IMGT definition as set out in Table 1) from a light chain variable region of an antibody described herein, e.g., an antibody chosen from 918, 350, 356, 358, 930, 373, 374, 376, 377, or 379; or encoded by a nucleotide sequence in Table 1; or a sequence substantially identical (e.g., having at least about 80%, 85%, 90%, 92%, 95%, 97%, 98%, or 99% sequence identity) to any of the aforesaid sequences; or which have at least one amino acid alteration, but not more than two, three or four alterations (e.g., substitutions, deletions, or insertions, e.g., conservative substitutions) relative to one, two, or three CDRs according to the IMGT definition shown in Table 1.

In certain embodiments, the anti-CD73 antibody molecule includes at least one, two, three, four, five, or six CDRs according to the IMGT definition (e.g., at least one, two, three, four, five, or six CDRs according to the IMGT definition as set out in Table 1) from the heavy and light chain variable regions of an antibody described herein, e.g., an antibody chosen from 918, 350, 356, 358, 930, 373, 374, 376, 377, or 379; or as described in Table 1; or encoded by a nucleotide sequence in Table 1; or a sequence substantially identical (e.g., having at least about 80%, 85%, 90%, 92%, 95%, 97%, 98%, or 99% sequence identity) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to the IMGT definition shown in Table 1.

In some embodiments, the anti-CD73 antibody molecule includes all six CDRs according to the IMGT definition (e.g., all six CDRs according to the IMGT definition as set out in Table 1) from the heavy and light chain variable regions of an antibody described herein, e.g., an antibody chosen from 918, 350, 356, 358, 930, 373, 374, 376, 377, or 379; or as described in Table 1; or encoded by a nucleotide sequence in Table 1; or a sequence substantially identical (e.g., having at least about 80%, 85%, 90%, 92%, 95%, 97%, 98%, or 99% sequence identity) to any of the aforesaid sequences; or which have at least one amino acid alteration, but not more than two, three or four alterations (e.g., substitutions, deletions, or insertions, e.g., conservative substitutions) relative to all six CDRs according to the IMGT definition shown in Table 1. In one embodiment, the anti-CD73 antibody molecule may include any CDR described herein.

The anti-CD73 antibody molecule can contain any combination of CDRs or hypervariable loops according to the Kabat and Chothia definitions.

In some embodiments, the anti-CD73 antibody molecule includes at least one, two, or three Chothia hypervariable loops from a heavy chain variable region of an antibody described herein, e.g., an antibody of Table 1, or at least the amino acids from those hypervariable loops that contact CD73.

In some embodiments, the anti-CD73 antibody molecule includes at least one, two, or three Chothia hypervariable loops from a light chain variable region of an antibody described herein, e.g., an antibody of Table 1, or at least the amino acids from those hypervariable loops that contact CD73.

In some embodiments, the anti-CD73 antibody molecule includes at least one, two, or three Kabat hypervariable loops from a heavy chain variable region of an antibody described herein, e.g., an antibody of Table 1, or at least the amino acids from those hypervariable loops that contact CD73.

In some embodiments, the anti-CD73 antibody molecule includes at least one, two, or three Kabat hypervariable loops from a light chain variable region of an antibody described herein, e.g., an antibody of Table 1, or at least the amino acids from those hypervariable loops that contact CD73.

In some embodiments, the anti-CD73 antibody molecule includes at least one, two, or three IMGT hypervariable loops from a heavy chain variable region of an antibody described herein, e.g., an antibody of Table 1, or at least the amino acids from those hypervariable loops that contact CD73.

In some embodiments, the anti-CD73 antibody molecule includes at least one, two, or three IMGT hypervariable loops from a light chain variable region of an antibody described herein, e.g., an antibody of Table 1, or at least the amino acids from those hypervariable loops that contact CD73.

In certain embodiments, the anti-CD73 antibody molecule includes at least one, two, three, four, five, or six hypervariable loops from the heavy and light chain variable regions of an antibody described herein, e.g., an antibody of Table 1, or at least the amino acids from those hypervariable loops that contact CD73.

In certain embodiments, the anti-CD73 antibody molecule includes all six hypervariable loops from the heavy and light chain variable regions of an antibody described herein, e.g., an antibody of Table 1, or at least the amino acids from those hypervariable loops that contact CD73, or at least the amino acids from those hypervariable loops that contact CD73, or closely related hypervariable loops, e.g., hypervariable loops which are identical or which have at least one amino acid alteration, but not more than two, three or four alterations (e.g., substitutions, e.g., conservative substitutions, deletions, or insertions).

In some embodiments, the anti-CD73 antibody molecule includes at least one, two, or three hypervariable loops that have the same canonical structures as the corresponding hypervariable loop of an antibody described herein, e.g., an antibody of Table 1, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references. In an embodiment, e.g., an embodiment comprising a variable region, CDR (e.g., Chothia, Kabat, or IMGT CDR), or other sequence referred to herein, e.g., in Table 1, the antibody molecule is a monospecific antibody molecule, a bispecific antibody molecule, or is an antibody molecule that comprises an antigen binding fragment of an antibody, e.g., a half antibody or antigen binding fragment of a half antibody.

In some embodiments, the heavy or light chain variable domain, or both, of the anti-CD73 antibody molecule includes an amino acid sequence, which is substantially identical to an amino acid disclosed herein, e.g., having at least about 80%, 85%, 90%, 92%, 95%, 97%, 98%, or 99% sequence identity to a variable region of an antibody described herein, e.g., an antibody chosen from 918, 350, 356, 358, 930, 373, 374, 376, 377, or 379; or as described in Table 1; or encoded by a nucleotide sequence in Table 1; or which differs at least 1 or 5 residues, but less than 40, 30, 20, or 10 residues, from a variable region of an antibody described herein.

In certain embodiments, the heavy or light chain variable region, or both, of the anti-CD73 antibody molecule includes an amino acid sequence encoded by a nucleic acid sequence described herein or a nucleic acid that hybridizes to a nucleic acid sequence described herein (e.g., a nucleic acid sequence as shown in Table 1) or its complement, e.g., under low stringency, medium stringency, or high stringency, or other hybridization condition described herein.

In certain embodiments, the anti-CD73 antibody molecule comprises at least one, two, three, or four antigen-binding regions, e.g., variable regions, having an amino acid sequence as set forth in Table 1, or a sequence substantially identical thereto (e.g., a sequence having at least about 85%, 90%, 95%, or 99% sequence identity thereto, or which differs by no more than 1, 2, 5, 10, or 15 amino acid residues from the sequences shown in Table 1. In certain embodiments, the anti-CD73 antibody molecule includes a VH and/or VL domain encoded by a nucleic acid having a nucleotide sequence that encodes an antibody of Table 1, or a sequence substantially identical to any one of the nucleotide sequences (e.g., a sequence having at least about 85%, 90%, 95%, or 99% sequence identity thereto, or which differs by no more than 3, 6, 15, 30, or 45 nucleotides from the sequences shown in Table 1).

In certain embodiments, the anti-CD73 antibody molecule comprises at least one, two, or three (e.g., all) CDRs from a heavy chain variable region having an amino acid sequence as set forth in Table 1, or a sequence substantially homologous thereto (e.g., a sequence having at least about 85%, 90%, 95%, or 99% sequence identity thereto, and/or having one, two, three or more substitutions, insertions or deletions, e.g., conserved substitutions). In some embodiments, the anti-CD73 antibody molecule comprises at least one, two, or three (e.g., all) CDRs from a light chain variable region having an amino acid sequence as set forth in Table 1, or a sequence substantially homologous thereto (e.g., a sequence having at least about 85%, 90%, 95%, or 99% sequence identity thereto, and/or having one, two, three or more substitutions, insertions or deletions, e.g., conserved substitutions). In certain embodiments, the anti-CD73 antibody molecule comprises at least one, two, three, four, five or six (e.g., all) CDRs from heavy and light chain variable regions having an amino acid sequence as set forth in Table 1, or a sequence substantially homologous thereto (e.g., a sequence having at least about 85%, 90%, 95%, or 99% sequence identity thereto, and/or having one, two, three or more substitutions, insertions or deletions, e.g., conserved substitutions).

In some embodiments, the anti-CD73 antibody molecule comprises at least one, two, or three (e.g., all) CDRs and/or hypervariable loops from a heavy chain variable region having an amino acid sequence of an antibody described herein, e.g., an antibody chosen from 918, 350, 356, 358, 930, 373, 374, 376, 377, or 379, as summarized in Table 1, or a sequence substantially identical thereto (e.g., a sequence having at least about 85%, 90%, 95%, or 99% sequence identity thereto, and/or having one, two, three or more substitutions, insertions or deletions, e.g., conserved substitutions). In certain embodiments, the anti-CD73 antibody molecule comprises at least one, two, or three (e.g., all) CDRs and/or hypervariable loops from a light chain variable region having an amino acid sequence of an antibody described herein, e.g., an antibody chosen from 918, 350, 356, 358, 930, 373, 374, 376, 377, or 379, as summarized in Table 1, or a sequence substantially identical thereto (e.g., a sequence having at least about 85%, 90%, 95%, or 99% sequence identity thereto, and/or having one, two, three or more substitutions, insertions or deletions, e.g., conserved substitutions). In some embodiments, the anti-CD73 antibody molecule comprises all six CDRs and/or hypervariable loops described herein, e.g., described in Table 1.

In some embodiments, the antibody molecule has a variable region that is identical in sequence, or which differs by 1, 2, 3, or 4 amino acids from a variable region described herein (e.g., an FR region disclosed herein).

In one embodiment, the anti-CD73 antibody molecule comprises a heavy chain variable region (VH) comprising a VHCDR3 amino acid sequence of GGLYGSGSYLSDFDL (SEQ ID NO: 37). In one embodiment, the anti-CD73 antibody molecule comprises a heavy chain variable region (VH) comprising a VHCDR3 amino acid sequence of ESQESPYNNWFDP (SEQ ID NO: 3).

In one embodiment, the anti-CD73 antibody molecule comprises a heavy chain variable region (VH) comprising a VHCDR1 amino acid sequence of SEQ ID NO: 88, a VHCDR2 amino acid sequence of SEQ ID NO: 89, and a VHCDR3 amino acid sequence of SEQ ID NO: 37; and a light chain variable region (VL) comprising a VLCDR1 amino acid sequence of SEQ ID NO: 48, a VLCDR2 amino acid sequence of SEQ ID NO: 49, and a VLCDR3 amino acid sequence of SEQ ID NO: 50, each disclosed in

Table 2. In one embodiment, the anti-CD73 antibody molecule comprises a heavy chain variable region (VH) comprising a VHCDR1 amino acid sequence of SEQ ID NO: 90, a VHCDR2 amino acid sequence of SEQ ID NO: 91, and a VHCDR3 amino acid sequence of SEQ ID NO: 3; and a light chain variable region (VL) comprising a VLCDR1 amino acid sequence of SEQ ID NO: 14, a VLCDR2 amino acid sequence of SEQ ID NO: 15, and a VLCDR3 amino acid sequence of SEQ ID NO: 16, each disclosed in Table 2.

In one embodiment, the anti-CD73 antibody molecule comprises a heavy chain variable region (VH) comprising a VHCDR1 amino acid sequence of SEQ ID NO: 122, a VHCDR2 amino acid sequence of SEQ ID NO: 123, and a VHCDR3 amino acid sequence of SEQ ID NO: 37; and a light chain variable region (VL) comprising a VLCDR1 amino acid sequence of SEQ ID NO: 51, a VLCDR2 amino acid sequence of SEQ ID NO: 52, and a VLCDR3 amino acid sequence of SEQ ID NO: 53, each disclosed in Table 2. In one embodiment, the anti-CD73 antibody molecule comprises a heavy chain variable region (VH) comprising a VHCDR1 amino acid sequence of SEQ ID NO: 124, a VHCDR2 amino acid sequence of SEQ ID NO: 125, and a VHCDR3 amino acid sequence of SEQ ID NO: 3; and a light chain variable region (VL) comprising a VLCDR1 amino acid sequence of SEQ ID NO: 17, a VLCDR2 amino acid sequence of SEQ ID NO: 18, and a VLCDR3 amino acid sequence of SEQ ID NO: 19, each disclosed in Table 2.

In one embodiment, the anti-CD73 antibody molecule comprises a heavy chain variable region (VH) comprising a VHCDR1 amino acid sequence of SEQ ID NO: 126, a VHCDR2 amino acid sequence of SEQ ID NO: 89, and a VHCDR3 amino acid sequence of SEQ ID NO: 37; and a light chain variable region (VL) comprising a VLCDR1 amino acid sequence of SEQ ID NO: 48, a VLCDR2 amino acid sequence of SEQ ID NO: 49, and a VLCDR3 amino acid sequence of SEQ ID NO: 50, each disclosed in Table 2. In one embodiment, the anti-CD73 antibody molecule comprises a heavy chain variable region (VH) comprising a VHCDR1 amino acid sequence of SEQ ID NO: 127, a VHCDR2 amino acid sequence of SEQ ID NO: 91, and a VHCDR3 amino acid sequence of SEQ ID NO: 3; and a light chain variable region (VL) comprising a VLCDR1 amino acid sequence of SEQ ID NO: 14, a VLCDR2 amino acid sequence of SEQ ID NO: 15, and a VLCDR3 amino acid sequence of SEQ ID NO: 16, each disclosed in Table 2.

In one embodiment, the anti-CD73 antibody molecule comprises a heavy chain variable region (VH) comprising a VHCDR1 amino acid sequence of SEQ ID NO: 128, a VHCDR2 amino acid sequence of SEQ ID NO: 129, and a VHCDR3 amino acid sequence of SEQ ID NO: 43; and a light chain variable region (VL) comprising a VLCDR1 amino acid sequence of SEQ ID NO: 54, a VLCDR2 amino acid sequence of SEQ ID NO: 52, and a VLCDR3 amino acid sequence of SEQ ID NO: 50, each disclosed in Table 2. In one embodiment, the anti-CD73 antibody molecule comprises a heavy chain variable region (VH) comprising a VHCDR1 amino acid sequence of SEQ ID NO: 130, a VHCDR2 amino acid sequence of SEQ ID NO: 131, and a VHCDR3 amino acid sequence of SEQ ID NO: 9; and a light chain variable region (VL) comprising a VLCDR1 amino acid sequence of SEQ ID NO: 20, a VLCDR2 amino acid sequence of SEQ ID NO: 18, and a VLCDR3 amino acid sequence of SEQ ID NO: 16, each disclosed in Table 2.

In one embodiment, the anti-CD73 antibody molecule comprises a heavy chain variable region (VH) comprising a VHCDR1 amino acid sequence of SEQ ID NO: 189, a VHCDR2 amino acid sequence of SEQ ID NO: 89, and a VHCDR3 amino acid sequence of SEQ ID NO: 43; and a light chain variable region (VL) comprising a VLCDR1 amino acid sequence of SEQ ID NO: 48, a VLCDR2 amino acid sequence of SEQ ID NO: 49, and a VLCDR3 amino acid sequence of SEQ ID NO: 50, each disclosed in Table 2. In one embodiment, the anti-CD73 antibody molecule comprises a heavy chain variable region (VH) comprising a VHCDR1 amino acid sequence of SEQ ID NO: 196, a VHCDR2 amino acid sequence of SEQ ID NO: 91, and a VHCDR3 amino acid sequence of SEQ ID NO: 9; and a light chain variable region (VL) comprising a VLCDR1 amino acid sequence of SEQ ID NO: 14, a VLCDR2 amino acid sequence of SEQ ID NO: 15, and a VLCDR3 amino acid sequence of SEQ ID NO: 16, each disclosed in Table 2.

In one embodiment, the anti-CD73 antibody molecule comprises a VH comprising a VHCDR1 amino acid sequence of SEQ ID NO: 38, a VHCDR2 amino acid sequence of SEQ ID NO: 36, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 72, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 38, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 137, a VHCDR2 amino acid sequence of SEQ ID NO: 136, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 137, a VHCDR2 amino acid sequence of SEQ ID NO: 146, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 137, a VHCDR2 amino acid sequence of SEQ ID NO: 154, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 61, a VHCDR2 amino acid sequence of SEQ ID NO: 60, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16. In one embodiment, the anti-CD73 antibody molecule comprises a VH comprising a VHCDR1 amino acid sequence of SEQ ID NO: 4, a VHCDR2 amino acid sequence of SEQ ID NO: 26, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16. In one embodiment, the anti-CD73 antibody molecule comprises a VH comprising a VHCDR1 amino acid sequence of SEQ ID NO: 4, a VHCDR2 amino acid sequence of SEQ ID NO: 2, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16. In one embodiment, the anti-CD73 antibody molecule comprises a VH comprising a VHCDR1 amino acid sequence of SEQ ID NO: 163, a VHCDR2 amino acid sequence of SEQ ID NO: 162, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16.

In one embodiment, the anti-CD73 antibody molecule comprises a VH comprising a VHCDR1 amino acid sequence of SEQ ID NO: 39, a VHCDR2 amino acid sequence of SEQ ID NO: 40, and a VHCDR3 amino acid sequence of SEQ ID NO: 37; and a VL comprising a VLCDR1 amino acid sequence of SEQ ID NO: 51, a VLCDR2 amino acid sequence of SEQ ID NO: 52, and a VLCDR3 amino acid sequence of SEQ ID NO: 53. In one embodiment, the anti-CD73 antibody molecule comprises a VH comprising a VHCDR1 amino acid sequence of SEQ ID NO: 73, a VHCDR2 amino acid sequence of SEQ ID NO: 74, and a VHCDR3 amino acid sequence of SEQ ID NO: 37; and a VL comprising a VLCDR1 amino acid sequence of SEQ ID NO: 51, a VLCDR2 amino acid sequence of SEQ ID NO: 52, and a VLCDR3 amino acid sequence of SEQ ID NO: 53. In one embodiment, the anti-CD73 antibody molecule comprises a VH comprising a VHCDR1 amino acid sequence of SEQ ID NO: 82, a VHCDR2 amino acid sequence of SEQ ID NO: 74, and a VHCDR3 amino acid sequence of SEQ ID NO: 37; and a VL comprising a VLCDR1 amino acid sequence of SEQ ID NO: 51, a VLCDR2 amino acid sequence of SEQ ID NO: 52, and a VLCDR3 amino acid sequence of SEQ ID NO: 53. In one embodiment, the anti-CD73 antibody molecule comprises a VH comprising a VHCDR1 amino acid sequence of SEQ ID NO: 138, a VHCDR2 amino acid sequence of SEQ ID NO: 139, and a VHCDR3 amino acid sequence of SEQ ID NO: 37; and a VL comprising a VLCDR1 amino acid sequence of SEQ ID NO: 51, a VLCDR2 amino acid sequence of SEQ ID NO: 52, and a VLCDR3 amino acid sequence of SEQ ID NO: 53. In one embodiment, the anti-CD73 antibody molecule comprises a VH comprising a VHCDR1 amino acid sequence of SEQ ID NO: 147, a VHCDR2 amino acid sequence of SEQ ID NO: 148, and a VHCDR3 amino acid sequence of SEQ ID NO: 37; and a VL comprising a VLCDR1 amino acid sequence of SEQ ID NO: 51, a VLCDR2 amino acid sequence of SEQ ID NO: 52, and a VLCDR3 amino acid sequence of SEQ ID NO: 53. In one embodiment, the anti-CD73 antibody molecule comprises a VH comprising a VHCDR1 amino acid sequence of SEQ ID NO: 155, a VHCDR2 amino acid sequence of SEQ ID NO: 156, and a VHCDR3 amino acid sequence of SEQ ID NO: 37; and a VL comprising a VLCDR1 amino acid sequence of SEQ ID NO: 51, a VLCDR2 amino acid sequence of SEQ ID NO: 52, and a VLCDR3 amino acid sequence of SEQ ID NO: 53. In one embodiment, the anti-CD73 antibody molecule comprises a VH comprising a VHCDR1 amino acid sequence of SEQ ID NO: 62, a VHCDR2 amino acid sequence of SEQ ID NO: 63, and a VHCDR3 amino acid sequence of SEQ ID NO: 3; and a VL comprising a VLCDR1 amino acid sequence of SEQ ID NO: 17, a VLCDR2 amino acid sequence of SEQ ID NO: 18, and a VLCDR3 amino acid sequence of SEQ ID NO: 19. In one embodiment, the anti-CD73 antibody molecule comprises a VH comprising a VHCDR1 amino acid sequence of SEQ ID NO: 27, a VHCDR2 amino acid sequence of SEQ ID NO: 28, and a VHCDR3 amino acid sequence of SEQ ID NO: 3; and a VL comprising a VLCDR1 amino acid sequence of SEQ ID NO: 17, a VLCDR2 amino acid sequence of SEQ ID NO: 18, and a VLCDR3 amino acid sequence of SEQ ID NO: 19. In one embodiment, the anti-CD73 antibody molecule comprises a VH comprising a VHCDR1 amino acid sequence of SEQ ID NO: 5, a VHCDR2 amino acid sequence of SEQ ID NO: 6, and a VHCDR3 amino acid sequence of SEQ ID NO: 3; and a VL comprising a VLCDR1 amino acid sequence of SEQ ID NO: 17, a VLCDR2 amino acid sequence of SEQ ID NO: 18, and a VLCDR3 amino acid sequence of SEQ ID NO: 19. In one embodiment, the anti-CD73 antibody molecule comprises a VH comprising a VHCDR1 amino acid sequence of SEQ ID NO: 164, a VHCDR2 amino acid sequence of SEQ ID NO: 165, and a VHCDR3 amino acid sequence of SEQ ID NO: 3; and a VL comprising a VLCDR1 amino acid sequence of SEQ ID NO: 17, a VLCDR2 amino acid sequence of SEQ ID NO: 18, and a VLCDR3 amino acid sequence of SEQ ID NO: 19.

In one embodiment, the anti-CD73 antibody molecule comprises a VH comprising a VHCDR1 amino acid sequence of SEQ ID NO: 35, a VHCDR2 amino acid sequence of SEQ ID NO: 36, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 70, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 81, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 135, a VHCDR2 amino acid sequence of SEQ ID NO: 136, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 145, a VHCDR2 amino acid sequence of SEQ ID NO: 146, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 153, a VHCDR2 amino acid sequence of SEQ ID NO: 154, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 59, a VHCDR2 amino acid sequence of SEQ ID NO: 60, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16. In one embodiment, the anti-CD73 antibody molecule comprises a VH comprising a VHCDR1 amino acid sequence of SEQ ID NO: 25, a VHCDR2 amino acid sequence of SEQ ID NO: 26, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16. In one embodiment, the anti-CD73 antibody molecule comprises a VH comprising a VHCDR1 amino acid sequence of SEQ ID NO: 1, a VHCDR2 amino acid sequence of SEQ ID NO: 2, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16. In one embodiment, the anti-CD73 antibody molecule comprises a VH comprising a VHCDR1 amino acid sequence of SEQ ID NO: 161, a VHCDR2 amino acid sequence of SEQ ID NO: 162, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16.

In one embodiment, the anti-CD73 antibody molecule comprises a VH comprising a VHCDR1 amino acid sequence of SEQ ID NO: 41, a VHCDR2 amino acid sequence of SEQ ID NO: 42, and a VHCDR3 amino acid sequence of SEQ ID NO: 43; and a VL comprising a VLCDR1 amino acid sequence of SEQ ID NO: 54, a VLCDR2 amino acid sequence of SEQ ID NO: 52, and a VLCDR3 amino acid sequence of SEQ ID NO: 50. In one embodiment, the anti-CD73 antibody molecule comprises a VH comprising a VHCDR1 amino acid sequence of SEQ ID NO: 75, a VHCDR2 amino acid sequence of SEQ ID NO: 76, and a VHCDR3 amino acid sequence of SEQ ID NO: 43; and a VL comprising a VLCDR1 amino acid sequence of SEQ ID NO: 54, a VLCDR2 amino acid sequence of SEQ ID NO: 52, and a VLCDR3 amino acid sequence of SEQ ID NO: 50. In one embodiment, the anti-CD73 antibody molecule comprises a VH comprising a VHCDR1 amino acid sequence of SEQ ID NO: 83, a VHCDR2 amino acid sequence of SEQ ID NO: 76, and a VHCDR3 amino acid sequence of SEQ ID NO: 43; and a VL comprising a VLCDR1 amino acid sequence of SEQ ID NO: 54, a VLCDR2 amino acid sequence of SEQ ID NO: 52, and a VLCDR3 amino acid sequence of SEQ ID NO: 50. In one embodiment, the anti-CD73 antibody molecule comprises a VH comprising a VHCDR1 amino acid sequence of SEQ ID NO: 140, a VHCDR2 amino acid sequence of SEQ ID NO: 141, and a VHCDR3 amino acid sequence of SEQ ID NO: 43; and a VL comprising a VLCDR1 amino acid sequence of SEQ ID NO: 54, a VLCDR2 amino acid sequence of SEQ ID NO: 52, and a VLCDR3 amino acid sequence of SEQ ID NO: 50. In one embodiment, the anti-CD73 antibody molecule comprises a VH comprising a VHCDR1 amino acid sequence of SEQ ID NO: 149, a VHCDR2 amino acid sequence of SEQ ID NO: 150, and a VHCDR3 amino acid sequence of SEQ ID NO: 43; and a VL comprising a VLCDR1 amino acid sequence of SEQ ID NO: 54, a VLCDR2 amino acid sequence of SEQ ID NO: 52, and a VLCDR3 amino acid sequence of SEQ ID NO: 50. In one embodiment, the anti-CD73 antibody molecule comprises a VH comprising a VHCDR1 amino acid sequence of SEQ ID NO: 157, a VHCDR2 amino acid sequence of SEQ ID NO: 158, and a VHCDR3 amino acid sequence of SEQ ID NO: 43; and a VL comprising a VLCDR1 amino acid sequence of SEQ ID NO: 54, a VLCDR2 amino acid sequence of SEQ ID NO: 52, and a VLCDR3 amino acid sequence of SEQ ID NO: 50. In one embodiment, the anti-CD73 antibody molecule comprises a VH comprising a VHCDR1 amino acid sequence of SEQ ID NO: 64, a VHCDR2 amino acid sequence of SEQ ID NO: 65, and a VHCDR3 amino acid sequence of SEQ ID NO: 9; and a VL comprising a VLCDR1 amino acid sequence of SEQ ID NO: 20, a VLCDR2 amino acid sequence of SEQ ID NO: 18, and a VLCDR3 amino acid sequence of SEQ ID NO: 16. In one embodiment, the anti-CD73 antibody molecule comprises a VH comprising a VHCDR1 amino acid sequence of SEQ ID NO: 29, a VHCDR2 amino acid sequence of SEQ ID NO: 30, and a VHCDR3 amino acid sequence of SEQ ID NO: 9; and a VL comprising a VLCDR1 amino acid sequence of SEQ ID NO: 20, a VLCDR2 amino acid sequence of SEQ ID NO: 18, and a VLCDR3 amino acid sequence of SEQ ID NO: 16. In one embodiment, the anti-CD73 antibody molecule comprises a VH comprising a VHCDR1 amino acid sequence of SEQ ID NO: 7, a VHCDR2 amino acid sequence of SEQ ID NO: 8, and a VHCDR3 amino acid sequence of SEQ ID NO: 9; and a VL comprising a VLCDR1 amino acid sequence of SEQ ID NO: 20, a VLCDR2 amino acid sequence of SEQ ID NO: 18, and a VLCDR3 amino acid sequence of SEQ ID NO: 16. In one embodiment, the anti-CD73 antibody molecule comprises a VH comprising a VHCDR1 amino acid sequence of SEQ ID NO: 166, a VHCDR2 amino acid sequence of SEQ ID NO: 167, and a VHCDR3 amino acid sequence of SEQ ID NO: 9; and a VL comprising a VLCDR1 amino acid sequence of SEQ ID NO: 20, a VLCDR2 amino acid sequence of SEQ ID NO: 18, and a VLCDR3 amino acid sequence of SEQ ID NO: 16.

In one embodiment, the anti-CD73 antibody molecule comprises a VH comprising a VHCDR1 amino acid sequence of SEQ ID NO: 190, a VHCDR2 amino acid sequence of SEQ ID NO: 36, and a VHCDR3 amino acid sequence of SEQ ID NO: 43; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 191, a VHCDR2 amino acid sequence of SEQ ID NO: 71, and a VHCDR3 amino acid sequence of SEQ ID NO: 43; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 192, a VHCDR2 amino acid sequence of SEQ ID NO: 71, and a VHCDR3 amino acid sequence of SEQ ID NO: 43; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 193, a VHCDR2 amino acid sequence of SEQ ID NO: 136, and a VHCDR3 amino acid sequence of SEQ ID NO: 43; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 194, a VHCDR2 amino acid sequence of SEQ ID NO: 146, and a VHCDR3 amino acid sequence of SEQ ID NO: 43; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 195, a VHCDR2 amino acid sequence of SEQ ID NO: 154, and a VHCDR3 amino acid sequence of SEQ ID NO: 43; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 197, a VHCDR2 amino acid sequence of SEQ ID NO: 60, and a VHCDR3 amino acid sequence of SEQ ID NO: 9; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16. In one embodiment, the anti-CD73 antibody molecule comprises a VH comprising a VHCDR1 amino acid sequence of SEQ ID NO: 198, a VHCDR2 amino acid sequence of SEQ ID NO: 26, and a VHCDR3 amino acid sequence of SEQ ID NO: 9; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16. In one embodiment, the anti-CD73 antibody molecule comprises a VH comprising a VHCDR1 amino acid sequence of SEQ ID NO: 199, a VHCDR2 amino acid sequence of SEQ ID NO: 2, and a VHCDR3 amino acid sequence of SEQ ID NO: 9; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16. In one embodiment, the anti-CD73 antibody molecule comprises a VH comprising a VHCDR1 amino acid sequence of SEQ ID NO: 200, a VHCDR2 amino acid sequence of SEQ ID NO: 162, and a VHCDR3 amino acid sequence of SEQ ID NO: 9; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16.

In other embodiments, the aforesaid antibodies comprise a heavy chain variable region comprising an amino acid sequence having at least about 85% (e.g., at least 90%, 95%, 97%, 98%, 99%, or 100%) sequence identity to any of SEQ ID NOs: 44, 77, 84, 142, 151, or 159. In other embodiments, the aforesaid antibodies comprise a heavy chain variable region comprising an amino acid sequence having at least about 85% (e.g., at least 90%, 95%, 97%, 98%, 99%, or 100%) sequence identity to any of SEQ ID NOs: 66, 31, 10, or 168.

In other embodiments, the aforesaid antibodies comprise a light chain variable region comprising an amino acid sequence having at least about 85% (e.g., at least 90%, 95%, 97%, 98%, 99%, or 100%) sequence identity to any of SEQ ID NOs: 55 or 21.

In other embodiments, the aforesaid antibodies comprise a heavy chain comprising an amino acid sequence having at least about 85% (e.g., at least 90%, 95%, 97%, 98%, 99%, or 100%) sequence identity to any of SEQ ID NOs: 46, 79, 86, 114, 116, or 117. In other embodiments, the aforesaid antibodies comprise a heavy chain comprising an amino acid sequence having at least about 85% (e.g., at least 90%, 95%, 97%, 98%, 99%, or 100%) sequence identity to any of SEQ ID NOs: 68, 33, 12, 115, 113, or 112.

In other embodiments, the aforesaid antibodies comprise a light chain comprising an amino acid sequence having at least about 85% (e.g., at least 90%, 95%, 97%, 98%, 99%, or 100%) sequence identity to any of SEQ ID NOs: 57 or 23.

In other embodiments, the antibody molecule comprises a heavy chain variable region comprising an amino acid sequence having at least about 85% (e.g., at least 90%, 95%, 97%, 98%, 99%, or 100%) sequence identity to SEQ ID NO: 44; and a light chain variable region comprising an amino acid sequence having at least about 85% (e.g., at least 90%, 95%, 97%, 98%, 99%, or 100%) sequence identity to SEQ ID NO: 55. In other embodiments, the antibody molecule comprises a heavy chain variable region comprising an amino acid sequence having at least about 85% (e.g., at least 90%, 95%, 97%, 98%, 99%, or 100%) sequence identity to SEQ ID NO: 77; and a light chain variable region comprising an amino acid sequence having at least about 85% (e.g., at least 90%, 95%, 97%, 98%, 99%, or 100%) sequence identity to SEQ ID NO: 55. In other embodiments, the antibody molecule comprises a heavy chain variable region comprising an amino acid sequence having at least about 85% (e.g., at least 90%, 95%, 97%, 98%, 99%, or 100%) sequence identity to SEQ ID NO: 84; and a light chain variable region comprising an amino acid sequence having at least about 85% (e.g., at least 90%, 95%, 97%, 98%, 99%, or 100%) sequence identity to SEQ ID NO: 55. In other embodiments, the antibody molecule comprises a heavy chain variable region comprising an amino acid sequence having at least about 85% (e.g., at least 90%, 95%, 97%, 98%, 99%, or 100%) sequence identity to SEQ ID NO: 142; and a light chain variable region comprising an amino acid sequence having at least about 85% (e.g., at least 90%, 95%, 97%, 98%, 99%, or 100%) sequence identity to SEQ ID NO: 55. In other embodiments, the antibody molecule comprises a heavy chain variable region comprising an amino acid sequence having at least about 85% (e.g., at least 90%, 95%, 97%, 98%, 99%, or 100%) sequence identity to SEQ ID NO: 151; and a light chain variable region comprising an amino acid sequence having at least about 85% (e.g., at least 90%, 95%, 97%, 98%, 99%, or 100%) sequence identity to SEQ ID NO: 55. In other embodiments, the antibody molecule comprises a heavy chain variable region comprising an amino acid sequence having at least about 85% (e.g., at least 90%, 95%, 97%, 98%, 99%, or 100%) sequence identity to SEQ ID NO: 159; and a light chain variable region comprising an amino acid sequence having at least about 85% (e.g., at least 90%, 95%, 97%, 98%, 99%, or 100%) sequence identity to SEQ ID NO: 55. In other embodiments, the antibody molecule comprises a heavy chain variable region comprising an amino acid sequence having at least about 85% (e.g., at least 90%, 95%, 97%, 98%, 99%, or 100%) sequence identity to SEQ ID NO: 66; and a light chain variable region comprising an amino acid sequence having at least about 85% (e.g., at least 90%, 95%, 97%, 98%, 99%, or 100%) sequence identity to SEQ ID NO: 21. In other embodiments, the antibody molecule comprises a heavy chain variable region comprising an amino acid sequence having at least about 85% (e.g., at least 90%, 95%, 97%, 98%, 99%, or 100%) sequence identity to SEQ ID NO: 31; and a light chain variable region comprising an amino acid sequence having at least about 85% (e.g., at least 90%, 95%, 97%, 98%, 99%, or 100%) sequence identity to SEQ ID NO: 21. In other embodiments, the antibody molecule comprises a heavy chain variable region comprising an amino acid sequence having at least about 85% (e.g., at least 90%, 95%, 97%, 98%, 99%, or 100%) sequence identity to SEQ ID NO: 10; and a light chain variable region comprising an amino acid sequence having at least about 85% (e.g., at least 90%, 95%, 97%, 98%, 99%, or 100%) sequence identity to SEQ ID NO: 21. In other embodiments, the antibody molecule comprises a heavy chain variable region comprising an amino acid sequence having at least about 85% (e.g., at least 90%, 95%, 97%, 98%, 99%, or 100%) sequence identity to SEQ ID NO: 168; and a light chain variable region comprising an amino acid sequence having at least about 85% (e.g., at least 90%, 95%, 97%, 98%, 99%, or 100%) sequence identity to SEQ ID NO: 21.

In other embodiments, the antibody molecule comprises a heavy chain comprising an amino acid sequence having at least about 85% (e.g., at least 90%, 95%, 97%, 98%, 99%, or 100%) sequence identity to SEQ ID NO: 46; and a light chain comprising an amino acid sequence having at least about 85% (e.g., at least 90%, 95%, 97%, 98%, 99%, or 100%) sequence identity to SEQ ID NO: 57. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 79; and a light chain comprising an amino acid sequence having at least about 85% (e.g., at least 90%, 95%, 97%, 98%, 99%, or 100%) sequence identity to SEQ ID NO: 57. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 86; and a light chain comprising an amino acid sequence having at least about 85% (e.g., at least 90%, 95%, 97%, 98%, 99%, or 100%) sequence identity to SEQ ID NO: 57. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 114; and a light chain comprising an amino acid sequence having at least about 85% (e.g., at least 90%, 95%, 97%, 98%, 99%, or 100%) sequence identity to SEQ ID NO: 57. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 116; and a light chain comprising an amino acid sequence having at least about 85% (e.g., at least 90%, 95%, 97%, 98%, 99%, or 100%) sequence identity to SEQ ID NO: 57. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 117; and a light chain comprising an amino acid sequence having at least about 85% (e.g., at least 90%, 95%, 97%, 98%, 99%, or 100%) sequence identity to SEQ ID NO: 57. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 68; and a light chain comprising an amino acid sequence having at least about 85% (e.g., at least 90%, 95%, 97%, 98%, 99%, or 100%) sequence identity to SEQ ID NO: 23. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 33; and a light chain comprising an amino acid sequence having at least about 85% (e.g., at least 90%, 95%, 97%, 98%, 99%, or 100%) sequence identity to SEQ ID NO: 23. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 12; and a light chain comprising an amino acid sequence having at least about 85% (e.g., at least 90%, 95%, 97%, 98%, 99%, or 100%) sequence identity to SEQ ID NO: 23. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 115; and a light chain comprising an amino acid sequence having at least about 85% (e.g., at least 90%, 95%, 97%, 98%, 99%, or 100%) sequence identity to SEQ ID NO: 23. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 113; and a light chain comprising an amino acid sequence having at least about 85% (e.g., at least 90%, 95%, 97%, 98%, 99%, or 100%) sequence identity to SEQ ID NO: 23. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 112; and a light chain comprising an amino acid sequence having at least about 85% (e.g., at least 90%, 95%, 97%, 98%, 99%, or 100%) sequence identity to SEQ ID NO: 23.

In other embodiments, the aforesaid antibody molecules are chosen from a fully antibody, a bispecific antibody, Fab, F(ab′)2, Fv, or a single chain Fv fragment (scFv).

In other embodiments, the aforesaid antibody molecules comprise a heavy chain constant region selected from IgG1, IgG2, IgG3, and IgG4.

In other embodiments, the aforesaid antibody molecules comprise a light chain constant region chosen from the light chain constant regions of kappa or lambda.

In some embodiments, the anti-CD73 antibody molecule comprises a heavy chain variable region, a light chain variable region, a heavy chain constant region, and/or a light chain constant region disclosed in Table 1. In some embodiments, the anti-CD73 antibody molecule comprises a heavy chain constant region, and/or a light chain constant region disclosed in Table 3. In some embodiments, the anti-CD73 antibody molecule comprises a heavy chain constant region comprising an amino acid sequence selected from the group consisting of SEQ ID NOs: 92-103, 119, and 120. In some embodiments, the anti-CD73 antibody molecule comprises a light chain constant region comprising the amino acid sequence of SEQ ID NO: 104.

Exemplary sequences of anti-CD73 antibodies are described in Tables 1 and 2 below.

TABLE 1
Amino acid and nucleotide sequences for exemplary anti-CD73 antibodies

| | | |
|---|---|---|
| 358 | | |
| SEQ ID NO: 1 (Combined) | HCDR1 | GGSISGRYWS |
| SEQ ID NO: 2 (Combined) | HCDR2 | YIYGTGSTNYNPSLKS |
| SEQ ID NO: 3 (Combined) | HCDR3 | ESQESPYNNWFDP |
| SEQ ID NO: 4 (Kabat) | HCDR1 | GRYWS |
| SEQ ID NO: 2 (Kabat) | HCDR2 | YIYGTGSTNYNPSLKS |
| SEQ ID NO: 3 (Kabat) | HCDR3 | ESQESPYNNWFDP |
| SEQ ID NO: 5 (Chothia) | HCDR1 | GGSISGR |
| SEQ ID NO: 6 (Chothia) | HCDR2 | YGTGS |
| SEQ ID NO: 3 (Chothia) | HCDR3 | ESQESPYNNWFDP |
| SEQ ID NO: 7 (IMGT) | HCDR1 | GGSISGRY |
| SEQ ID NO: 8 (IMGT) | HCDR2 | IYGTGST |
| SEQ ID NO: 9 (IMGT) | HCDR3 | ARESQESPYNNWFDP |
| SEQ ID NO: 199 | HCDR1 | GSISGRYWS |
| (Alternative) | | |
| SEQ ID NO: 2 | HCDR2 | YIYGTGSTNYNPSLKS |
| (Alternative) | | |
| SEQ ID NO: 9 | HCDR3 | ARESQESPYNNWFDP |
| (Alternative) | | |
| SEQ ID NO: 10 | VH | QVQLQESGPGLVKPSETLSLTCTVSGGSISGRYWSWIRQ |
| | | PPGKGLEWIGYIYGTGSTNYNPSLKSRVTISVDTSKNQFS |
| | | LKLSSVTAADTAVYYCARESQESPYNNWFDPWGQGTL |
| | | VTVSS |
| SEQ ID NO: 11 | DNA VH | CAAGTGCAGCTGCAGGAATCTGGCCCTGGCCTGGTCA |
| | | AGCCCTCCGAGACACTGTCCCTGACCTGCACCGTGTC |
| | | CGGCGGCTCCATCTCCGGCCGGTACTGGTCTTGGATC |
| | | CGGCAGCCTCCCGGCAAGGGCCTGGAATGGATCGGC |
| | | TACATCTACGGCACCGGCTCCACCAACTACAACCCCA |
| | | GCCTGAAGTCCAGAGTGACCATCTCCGTGGACACCTC |
| | | CAAGAACCAGTTCTCCCTGAAGCTGTCCTCCGTGACC |
| | | GCCGCTGACACCGCCGTGTACTACTGCGCCAGAGAGT |
| | | CCCAGGAATCCCCTTACAACAATTGGTTCGACCCCTG |
| | | GGGCCAGGGCACCCTGGTCACCGTGTCCTCT |
| SEQ ID NO: 12 | 358.A | QVQLQESGPGLVKPSETLSLTCTVSGGSISGRYWSWIRQ |
| | Heavy | PPGKGLEWIGYIYGTGSTNYNPSLKSRVTISVDTSKNQFS |
| | Chain | LKLSSVTAADTAVYYCARESQESPYNNWFDPWGQGTL |
| | | VTVSSASTKGPSVFPLAPCSRSTSESTAALGCLVKDYFPE |
| | | PVTVSWNSGALTSGVHTFPAVLQSSGLYSLSSVVTVPSS |
| | | SLGTKTYTCNVDHKPSNTKVDKRVESKYGPPCPPCPAP |
| | | EFLGGPSVFLFPPKPKDTLMISRTPEVTCVVVDVSQEDPE |
| | | VQFNWYVDGVEVHNAKTKPREEQFNSTYRVVSVLTVL |
| | | HQDWLNGKEYKCKVSNKGLPSSIEKTISKAKGQPREPQ |
| | | VYTLPPSQEEMTKNQVSLTCLVKGFYPSDIAVEWESNG |
| | | QPENNYKTTPPVLDSDGSFFLYSRLTVDKSRWQEGNVF |
| | | SCSVMHEALHNHYTQKSLSLSLGX, |
| | | wherein X is K or absent. |
| SEQ ID NO: 13 | 358.A | CAAGTGCAGCTGCAGGAATCTGGCCCTGGCCTGGTCA |
| | DNA | AGCCCTCCGAGACACTGTCCCTGACCTGCACCGTGTC |
| | Heavy | CGGCGGCTCCATCTCCGGCCGGTACTGGTCTTGGATC |
| | Chain | CGGCAGCCTCCCGGCAAGGGCCTGGAATGGATCGGC |
| | | TACATCTACGGCACCGGCTCCACCAACTACAACCCCA |
| | | GCCTGAAGTCCAGAGTGACCATCTCCGTGGACACCTC |
| | | CAAGAACCAGTTCTCCCTGAAGCTGTCCTCCGTGACC |
| | | GCCGCTGACACCGCCGTGTACTACTGCGCCAGAGAGT |
| | | CCCAGGAATCCCCTTACAACAATTGGTTCGACCCCTG |
| | | GGGCCAGGGCACCCTGGTCACCGTGTCCTCTGCTTCC |
| | | ACCAAGGGCCCCTCCGTGTTCCCTCTGGCCCCTTGCTC |
| | | CCGGTCCACCTCCGAGTCTACCGCCGCTCTGGGCTGC |
| | | CTCGTGAAGGACTACTTCCCCGAGCCCGTGACCGTGT |
| | | CCTGGAACTCTGGCGCCCTGACCTCCGGCGTGCACAC |
| | | CTTCCCTGCCGTGCTGCAGTCCTCCGGCCTGTACTCCC |
| | | TGTCCAGCGTCGTGACCGTGCCCTCCTCCAGCCTGGG |
| | | CACCAAGACCTACACCTGTAACGTGGACCACAAGCCC |
| | | TCCAACACCAAAGTGGACAAGCGGGTGGAATCTAAG |
| | | TACGGCCCTCCCTGCCCTCCTTGCCCTGCCCCTGAGTT |
| | | CCTGGGCGGACCTTCCGTGTTCCTGTTCCCTCCAAAG |
| | | CCCAAGGACACCCTGATGATCTCCCGGACCCCTGAAG |
| | | TGACCTGCGTGGTGGTGGACGTGTCCCAGGAAGATCC |
| | | CGAAGTCCAGTTCAATTGGTACGTGGACGGCGTGGAA |
| | | GTGCACAACGCCAAGACCAAGCCCAGAGAGGAACAG |
| | | TTCAACTCCACCTACCGGGTGGTGTCCGTGCTGACCG |
| | | TGCTGCACCAGGACTGGCTGAACGGCAAAGAGTACA |
| | | AGTGCAAAGTGTCCAACAAGGGCCTGCCCTCCAGCAT |
| | | CGAAAAGACCATCTCCAAGGCCAAGGGCCAGCCCCG |
| | | CGAGCCCCAAGTGTACACCCTGCCTCCCAGCCAGGAA |
| | | GAGATGACCAAGAATCAAGTGTCCCTGACTTGTCTGG |
| | | TCAAGGGCTTCTACCCCTCCGATATCGCCGTGGAGTG |
| | | GGAGTCCAACGGCCAGCCCGAGAACAACTACAAGAC |
| | | CACCCCTCCCGTGCTGGACTCCGACGGCTCCTTCTTCC |
| | | TGTACTCTCGGCTGACCGTGGACAAGTCCCGGTGGCA |
| | | GGAAGGCAACGTCTTCTCCTGCTCCGTGATGCACGAG |
| | | GCCCTGCACAACCACTACACCCAGAAGTCCCTGTCCC |
| | | TGTCTCTGGGCX₁X₂X₃, |
| | | wherein X₁ is A, X₂ is A, and X₃ is G; or X₁ is |
| | | absent, X₂ is absent, and X₃ is absent. |
| SEQ ID NO: 112 | 358.B | QVQLQESGPGLVKPSETLSLTCTVSGGSISGRYWSWIRQ |
| | Heavy | PPGKGLEWIGYIYGTGSTNYNPSLKSRVTISVDTSKNQFS |
| | Chain | LKLSSVTAADTAVYYCARESQESPYNNWFDPWGQGTL |
| | | VTVSSASTKGPSVFPLAPCSRSTSESTAALGCLVKDYFPE |
| | | PVTVSWNSGALTSGVHTFPAVLQSSGLYSLSSVVTVPSS |
| | | SLGTKTYTCNVDHKPSNTKVDKRVESKYGPPCPPCPAP |
| | | EFEGGPSVFLFPPKPKDTLMISRTPEVTCVVVDVSQEDPE |
| | | VQFNWYVDGVEVHNAKTKPREEQFNSTYRVVSVLTVL |
| | | HQDWLNGKEYKCKVSNKGLPSSIEKTISKAKGQPREPQ |
| | | VYTLPPSQEEMTKNQVSLTCLVKGFYPSDIAVEWESNG |
| | | QPENNYKTTPPVLDSDGSFFLYSRLTVDKSRWQEGNVF |
| | | SCSVMHEALHNHYTQKSLSLSLGX, |
| | | wherein X is K or absent |
| SEQ ID NO: 14 | LCDR1 | RASQGISSWLA |
| (Combined) | | |
| SEQ ID NO: 15 | LCDR2 | AASSLQS |
| (Combined) | | |
| SEQ ID NO: 16 | LCDR3 | QQGNSFPRT |
| (Combined) | | |
| SEQ ID NO: 14 (Kabat) | LCDR1 | RASQGISSWLA |
| SEQ ID NO: 15 (Kabat) | LCDR2 | AASSLQS |
| SEQ ID NO: 16 (Kabat) | LCDR3 | QQGNSFPRT |
| SEQ ID NO: 17 (Chothia) | LCDR1 | SQGISSW |
| SEQ ID NO: 18 (Chothia) | LCDR2 | AAS |
| SEQ ID NO: 19 (Chothia) | LCDR3 | GNSFPR |
| SEQ ID NO: 20 (IMGT) | LCDR1 | QGISSW |
| SEQ ID NO: 18 (IMGT) | LCDR2 | AAS |
| SEQ ID NO: 16 (IMGT) | LCDR3 | QQGNSFPRT |
| SEQ ID NO: 14 | LCDR1 | RASQGISSWLA |
| (Alternative) | | |
| SEQ ID NO: 15 | LCDR2 | AASSLQS |
| (Alternative) | | |
| SEQ ID NO: 16 | LCDR3 | QQGNSFPRT |
| (Alternative) | | |
| SEQ ID NO: 21 | VL | DIQMTQSPSSVSASVGDRVTITCRASQGISSWLAWYQQ |
| | | KPGKAPKLLIYAASSLQSGVPSRFSGSGSGTDFTLTISSL |
| | | QPEDFATYYCQQGNSFPRTFGGGTKVEIK |
| SEQ ID NO: 22 | DNA VL | GACATCCAGATGACCCAGAGCCCCTCCTCCGTGTCCG |
| | | CCTCCGTGGGCGACAGAGTGACCATCACCTGTCGGGC |
| | | CTCCCAGGGCATCTCCAGCTGGCTGGCCTGGTATCAG |
| | | CAGAAGCCCGGCAAGGCCCCTAAGCTGCTGATCTACG |
| | | CCGCCTCCAGCCTGCAGTCCGGCGTGCCCTCCAGATT |
| | | CTCCGGCTCTGGCTCCGGCACCGACTTCACCCTGACC |
| | | ATCAGCTCCCTGCAGCCCGAGGACTTCGCCACCTACT |
| | | ACTGCCAGCAGGGCAACTCCTTCCCTCGGACCTTCGG |
| | | CGGAGGCACCAAAGTGGAAATCAAG |
| SEQ ID NO: 23 | Light | DIQMTQSPSSVSASVGDRVTITCRASQGISSWLAWYQQ |
| | Chain | KPGKAPKLLIYAASSLQSGVPSRFSGSGSGTDFTLTISSL |
| | | QPEDFATYYCQQGNSFPRTFGGGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYA |
| | | CEVTHQGLSSPVTKSFNRGEC |
| SEQ ID NO: 24 | DNA | GACATCCAGATGACCCAGAGCCCCTCCTCCGTGTCCG |
| | Light | CCTCCGTGGGCGACAGAGTGACCATCACCTGTCGGGC |
| | Chain | CTCCCAGGGCATCTCCAGCTGGCTGGCCTGGTATCAG |
| | | CAGAAGCCCGGCAAGGCCCCTAAGCTGCTGATCTACG |
| | | CCGCCTCCAGCCTGCAGTCCGGCGTGCCCTCCAGATT |
| | | CTCCGGCTCTGGCTCCGGCACCGACTTCACCCTGACC |
| | | ATCAGCTCCCTGCAGCCCGAGGACTTCGCCACCTACT |
| | | ACTGCCAGCAGGGCAACTCCTTCCCTCGGACCTTCGG |
| | | CGGAGGCACCAAAGTGGAAATCAAGCGTACGGTGGC |
| | | CGCTCCCTCCGTGTTCATCTTCCCACCCTCCGACGAGC |
| | | AGCTGAAGTCCGGCACCGCCTCCGTCGTGTGCCTGCT |
| | | GAACAACTTCTACCCTCGCGAGGCCAAAGTGCAGTGG |
| | | AAAGTGGACAACGCCCTGCAGTCCGGCAACTCCCAG |
| | | GAATCCGTCACCGAGCAGGACTCCAAGGACAGCACC |
| | | TACTCCCTGTCCTCCACCCTGACCCTGTCCAAGGCCG |
| | | ACTACGAGAAGCACAAAGTGTACGCCTGCGAAGTGA |
| | | CCCACCAGGGCCTGTCCAGCCCCGTGACCAAGTCCTT |
| | | CAACCGGGGCGAGTGC |
| 356 | | |
| SEQ ID NO: 25 | HCDR1 | GGSIEGRYWS |
| (Combined) | | |
| SEQ ID NO: 26 | HCDR2 | YIYGSGSTKYNPSLKS |
| (Combined) | | |
| SEQ ID NO: 3 (Combined) | HCDR3 | ESQESPYNNWFDP |
| SEQ ID NO: 4 (Kabat) | HCDR1 | GRYWS |
| SEQ ID NO: 26 (Kabat) | HCDR2 | YIYGSGSTKYNPSLKS |
| SEQ ID NO: 3 (Kabat) | HCDR3 | ESQESPYNNWFDP |
| SEQ ID NO: 27 (Chothia) | HCDR1 | GGSIEGR |
| SEQ ID NO: 28 (Chothia) | HCDR2 | YGSGS |
| SEQ ID NO: 3 (Chothia) | HCDR3 | ESQESPYNNWFDP |
| SEQ ID NO: 29 (IMGT) | HCDR1 | GGSIEGRY |
| SEQ ID NO: 30 (IMGT) | HCDR2 | IYGSGST |
| SEQ ID NO: 9 (IMGT) | HCDR3 | ARESQESPYNNWFDP |
| SEQ ID NO: 198 | HCDR1 | GSIEGRYWS |
| (Alternative) | | |
| SEQ ID NO: 26 | HCDR2 | YIYGSGSTKYNPSLKS |
| (Alternative) | | |
| SEQ ID NO: 9 | HCDR3 | ARESQESPYNNWFDP |
| (Alternative) | | |
| SEQ ID NO: 31 | VH | QVQLQESGPGLVKPSETLSLTCTVSGGSIEGRYWSWIRQ |
| | | PPGKGLEWIGYIYGSGSTKYNPSLKSRVTISVDTSKNQFS |
| | | LKLSSVTAADTAVYYCARESQESPYNNWFDPWGQGTL |
| | | VTVSS |
| SEQ ID NO: 32 | DNA VH | CAAGTGCAGCTGCAGGAATCTGGCCCTGGCCTGGTCA |
| | | AGCCCTCCGAGACACTGTCCCTGACCTGCACCGTGTC |
| | | CGGCGGCTCTATCGAGGGCCGGTACTGGTCCTGGATC |
| | | CGGCAGCCTCCTGGCAAGGGCCTGGAATGGATCGGCT |
| | | ACATCTACGGCTCCGGCTCCACCAAGTACAACCCCAG |
| | | CCTGAAGTCCAGAGTGACCATCTCCGTGGACACCTCC |
| | | AAGAACCAGTTCTCCCTGAAGCTGTCCTCCGTGACCG |
| | | CCGCTGACACCGCCGTGTACTACTGCGCCAGAGAGTC |
| | | CCAGGAATCCCCTTACAACAATTGGTTCGACCCCTGG |
| | | GGCCAGGGCACCCTGGTCACCGTGTCCTCT |
| SEQ ID NO: 33 | 356.A | QVQLQESGPGLVKPSETLSLTCTVSGGSIEGRYWSWIRQ |
| | Heavy | PPGKGLEWIGYIYGSGSTKYNPSLKSRVTISVDTSKNQFS |
| | Chain | LKLSSVTAADTAVYYCARESQESPYNNWFDPWGQGTL |
| | | VTVSSASTKGPSVFPLAPCSRSTSESTAALGCLVKDYFPE |
| | | PVTVSWNSGALTSGVHTFPAVLQSSGLYSLSSVVTVPSS |
| | | SLGTKTYTCNVDHKPSNTKVDKRVESKYGPPCPPCPAP |
| | | EFLGGPSVFLFPPKPKDTLMISRTPEVTCVVVDVSQEDPE |
| | | VQFNWYVDGVEVHNAKTKPREEQFNSTYRVVSVLTVL |
| | | HQDWLNGKEYKCKVSNKGLPSSIEKTISKAKGQPREPQ |
| | | VYTLPPSQEEMTKNQVSLTCLVKGFYPSDIAVEWESNG |
| | | QPENNYKTTPPVLDSDGSFFLYSRLTVDKSRWQEGNVF |
| | | SCSVMHEALHNHYTQKSLSLSLGX, |
| | | wherein X is K or absent |
| SEQ ID NO: 34 | 356.A | CAAGTGCAGCTGCAGGAATCTGGCCCTGGCCTGGTCA |
| | DNA | AGCCCTCCGAGACACTGTCCCTGACCTGCACCGTGTC |
| | Heavy | CGGCGGCTCTATCGAGGGCCGGTACTGGTCCTGGATC |
| | Chain | CGGCAGCCTCCTGGCAAGGGCCTGGAATGGATCGGCT |
| | | ACATCTACGGCTCCGGCTCCACCAAGTACAACCCCAG |
| | | CCTGAAGTCCAGAGTGACCATCTCCGTGGACACCTCC |
| | | AAGAACCAGTTCTCCCTGAAGCTGTCCTCCGTGACCG |
| | | CCGCTGACACCGCCGTGTACTACTGCGCCAGAGAGTC |
| | | CCAGGAATCCCCTTACAACAATTGGTTCGACCCCTGG |
| | | GGCCAGGGCACCCTGGTCACCGTGTCCTCTGCCTCCA |
| | | CCAAGGGCCCCTCCGTGTTCCCTCTGGCCCCTTGCTCC |
| | | CGGTCCACCTCCGAGTCTACCGCCGCTCTGGGCTGCC |
| | | TCGTGAAGGACTACTTCCCCGAGCCCGTGACCGTGTC |
| | | CTGGAACTCTGGCGCCCTGACCTCCGGCGTGCACACC |
| | | TTCCCTGCCGTGCTGCAGTCCTCCGGCCTGTACTCCCT |
| | | GTCCAGCGTCGTGACCGTGCCCTCCTCCAGCCTGGGC |
| | | ACCAAGACCTACACCTGTAACGTGGACCACAAGCCCT |
| | | CCAACACCAAAGTGGACAAGCGGGTGGAATCTAAGT |
| | | ACGGCCCTCCCTGCCCTCCTTGCCCTGCCCCTGAGTTC |
| | | CTGGGCGGACCTTCCGTGTTCCTGTTCCCTCCAAAGC |
| | | CCAAGGACACCCTGATGATCTCCCGGACCCCTGAAGT |
| | | GACCTGCGTGGTGGTGGACGTGTCCCAGGAAGATCCC |
| | | GAAGTCCAGTTCAATTGGTACGTGGACGGCGTGGAA |
| | | GTGCACAACGCCAAGACCAAGCCCAGAGAGGAACAG |
| | | TTCAACTCCACCTACCGGGTGGTGTCCGTGCTGACCG |
| | | TGCTGCACCAGGACTGGCTGAACGGCAAAGAGTACA |
| | | AGTGCAAAGTGTCCAACAAGGGCCTGCCCTCCAGCAT |
| | | CGAAAAGACCATCTCCAAGGCCAAGGGCCAGCCCCG |
| | | CGAGCCCCAAGTGTACACCCTGCCTCCCAGCCAGGAA |
| | | GAGATGACCAAGAATCAAGTGTCCCTGACTTGTCTGG |
| | | TCAAGGGCTTCTACCCCTCCGATATCGCCGTGGAGTG |
| | | GGAGTCCAACGGCCAGCCCGAGAACAACTACAAGAC |
| | | CACCCCTCCCGTGCTGGACTCCGACGGCTCCTTCTTCC |
| | | TGTACTCTCGGCTGACCGTGGACAAGTCCCGGTGGCA |
| | | GGAAGGCAACGTCTTCTCCTGCTCCGTGATGCACGAG |
| | | GCCCTGCACAACCACTACACCCAGAAGTCCCTGTCCC |
| | | TGTCTCTGGGCX₁X₂X₃, |
| | | wherein X₁ is A, X₂ is A, and X₃ is G; or X₁ is |
| | | absent, X₂ is absent, and X₃ is absent. |
| SEQ ID NO: 113 | 356.B | QVQLQESGPGLVKPSETLSLTCTVSGGSIEGRYWSWIRQ |
| | Heavy | PPGKGLEWIGYIYGSGSTKYNPSLKSRVTISVDTSKNQFS |
| | chain | LKLSSVTAADTAVYYCARESQESPYNNWFDPWGQGTL |
| | | VTVSSASTKGPSVFPLAPCSRSTSESTAALGCLVKDYFPE |
| | | PVTVSWNSGALTSGVHTFPAVLQSSGLYSLSSVVTVPSS |
| | | SLGTKTYTCNVDHKPSNTKVDKRVESKYGPPCPPCPAP |
| | | EFEGGPSVFLFPPKPKDTLMISRTPEVTCVVVDVSQEDPE |
| | | VQFNWYVDGVEVHNAKTKPREEQFNSTYRVVSVLTVL |
| | | HQDWLNGKEYKCKVSNKGLPSSIEKTISKAKGQPREPQ |
| | | VYTLPPSQEEMTKNQVSLTCLVKGFYPSDIAVEWESNG |
| | | QPENNYKTTPPVLDSDGSFFLYSRLTVDKSRWQEGNVF |
| | | SCSVMHEALHNHYTQKSLSLSLGX, |
| | | wherein X is K or absent |
| SEQ ID NO: 14 | LCDR1 | RASQGISSWLA |
| (Combined) | | |
| SEQ ID NO: 15 | LCDR2 | AASSLQS |
| (Combined) | | |
| SEQ ID NO: 16 | LCDR3 | QQGNSFPRT |
| (Combined) | | |
| SEQ ID NO: 14 (Kabat) | LCDR1 | RASQGISSWLA |
| SEQ ID NO: 15 (Kabat) | LCDR2 | AASSLQS |
| SEQ ID NO: 16 (Kabat) | LCDR3 | QQGNSFPRT |
| SEQ ID NO: 17 (Chothia) | LCDR1 | SQGISSW |
| SEQ ID NO: 18 (Chothia) | LCDR2 | AAS |
| SEQ ID NO: 19 (Chothia) | LCDR3 | GNSFPR |
| SEQ ID NO: 20 (IMGT) | LCDR1 | QGISSW |
| SEQ ID NO: 18 (IMGT) | LCDR2 | AAS |
| SEQ ID NO: 16 (IMGT) | LCDR3 | QQGNSFPRT |
| SEQ ID NO: 14 | LCDR1 | RASQGISSWLA |
| (Alternative) | | |
| SEQ ID NO: 15 | LCDR2 | AASSLQS |
| (Alternative) | | |
| SEQ ID NO: 16 | LCDR3 | QQGNSFPRT |
| (Alternative) | | |
| SEQ ID NO: 21 | VL | DIQMTQSPSSVSASVGDRVTITCRASQGISSWLAWYQQ |
| | | KPGKAPKLLIYAASSLQSGVPSRFSGSGSGTDFTLTISSL |
| | | QPEDFATYYCQQGNSFPRTFGGGTKVEIK |
| SEQ ID NO: 22 | DNA VL | GACATCCAGATGACCCAGAGCCCCTCCTCCGTGTCCG |
| | | CCTCCGTGGGCGACAGAGTGACCATCACCTGTCGGGC |
| | | CTCCCAGGGCATCTCCAGCTGGCTGGCCTGGTATCAG |
| | | CAGAAGCCCGGCAAGGCCCCTAAGCTGCTGATCTACG |
| | | CCGCCTCCAGCCTGCAGTCCGGCGTGCCCTCCAGATT |
| | | CTCCGGCTCTGGCTCCGGCACCGACTTCACCCTGACC |
| | | ATCAGCTCCCTGCAGCCCGAGGACTTCGCCACCTACT |
| | | ACTGCCAGCAGGGCAACTCCTTCCCTCGGACCTTCGG |
| | | CGGAGGCACCAAAGTGGAAATCAAG |
| SEQ ID NO: 23 | Light | DIQMTQSPSSVSASVGDRVTITCRASQGISSWLAWYQQ |
| | Chain | KPGKAPKLLIYAASSLQSGVPSRFSGSGSGTDFTLTISSL |
| | | QPEDFATYYCQQGNSFPRTFGGGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYA |
| | | CEVTHQGLSSPVTKSFNRGEC |
| SEQ ID NO: 24 | DNA | GACATCCAGATGACCCAGAGCCCCTCCTCCGTGTCCG |
| | Light | CCTCCGTGGGCGACAGAGTGACCATCACCTGTCGGGC |
| | Chain | CTCCCAGGGCATCTCCAGCTGGCTGGCCTGGTATCAG |
| | | CAGAAGCCCGGCAAGGCCCCTAAGCTGCTGATCTACG |
| | | CCGCCTCCAGCCTGCAGTCCGGCGTGCCCTCCAGATT |
| | | CTCCGGCTCTGGCTCCGGCACCGACTTCACCCTGACC |
| | | ATCAGCTCCCTGCAGCCCGAGGACTTCGCCACCTACT |
| | | ACTGCCAGCAGGGCAACTCCTTCCCTCGGACCTTCGG |
| | | CGGAGGCACCAAAGTGGAAATCAAGCGTACGGTGGC |
| | | CGCTCCCTCCGTGTTCATCTTCCCACCCTCCGACGAGC |
| | | AGCTGAAGTCCGGCACCGCCTCCGTCGTGTGCCTGCT |
| | | GAACAACTTCTACCCTCGCGAGGCCAAAGTGCAGTGG |
| | | AAAGTGGACAACGCCCTGCAGTCCGGCAACTCCCAG |
| | | GAATCCGTCACCGAGCAGGACTCCAAGGACAGCACC |
| | | TACTCCCTGTCCTCCACCCTGACCCTGTCCAAGGCCG |
| | | ACTACGAGAAGCACAAAGTGTACGCCTGCGAAGTGA |
| | | CCCACCAGGGCCTGTCCAGCCCCGTGACCAAGTCCTT |
| | | CAACCGGGGCGAGTGC |
| 373 | | |
| SEQ ID NO: 35 | HCDR1 | GFTFHRYAMS |
| (Combined) | | |
| SEQ ID NO: 36 | HCDR2 | AISGSGMNTYYADSVKG |
| (Combined) | | |
| SEQ ID NO: 37 | HCDR3 | GGLYGSGSYLSDFDL |
| (Combined) | | |
| SEQ ID NO: 38 (Kabat) | HCDR1 | RYAMS |
| SEQ ID NO: 36 (Kabat) | HCDR2 | AISGSGMNTYYADSVKG |
| SEQ ID NO: 37 (Kabat) | HCDR3 | GGLYGSGSYLSDFDL |
| SEQ ID NO: 39 (Chothia) | HCDR1 | GFTFHRY |
| SEQ ID NO: 40 (Chothia) | HCDR2 | SGSGMN |
| SEQ ID NO: 37 (Chothia) | HCDR3 | GGLYGSGSYLSDFDL |
| SEQ ID NO: 41 (IMGT) | HCDR1 | GFTFHRYA |
| SEQ ID NO: 42 (IMGT) | HCDR2 | ISGSGMNT |
| SEQ ID NO: 43 (IMGT) | HCDR3 | ARGGLYGSGSYLSDFDL |
| SEQ ID NO: 190 | HCDR1 | FTFHRYAMS |
| (Alternative) | | |
| SEQ ID NO: 36 | HCDR2 | AISGSGMNTYYADSVKG |
| (Alternative) | | |
| SEQ ID NO: 43 | HCDR3 | ARGGLYGSGSYLSDFDL |
| (Alternative) | | |
| SEQ ID NO: 44 | VH | EVQLLESGGGLVQPGGSLRLSCAASGFTFHRYAMSWVR |
| | | QAPGKGLEWVSAISGSGMNTYYADSVKGRFTISRDNSK |
| | | NTLYLQMNSLRAEDTAVYYCARGGLYGSGSYLSDFDL |
| | | WGRGTLVTVSS |
| SEQ ID NO: 45 | DNA VH | GAAGTGCAGCTGCTGGAATCTGGCGGCGGACTGGTG |
| | | CAGCCTGGCGGCTCCCTGAGACTGTCTTGCGCCGCCT |
| | | CCGGCTTCACCTTCCACAGATACGCCATGTCCTGGGT |
| | | CCGACAGGCCCCTGGCAAGGGCCTGGAGTGGGTGTC |
| | | CGCCATCTCCGGCTCCGGCATGAACACCTACTACGCC |
| | | GACTCCGTGAAGGGCCGGTTCACCATCTCCCGGGACA |
| | | ACTCCAAGAACACCCTGTACCTGCAGATGAACTCCCT |
| | | GCGGGCCGAGGACACCGCCGTGTACTACTGTGCCAG |
| | | AGGCGGCCTGTACGGCTCCGGCTCCTACCTGTCCGAC |
| | | TTCGACCTGTGGGGCAGAGGCACCCTGGTCACCGTGT |
| | | CCTCC |
| SEQ ID NO: 46 | 373.A | EVQLLESGGGLVQPGGSLRLSCAASGFTFHRYAMSWVR |
| | Heavy | QAPGKGLEWVSAISGSGMNTYYADSVKGRFTISRDNSK |
| | Chain | NTLYLQMNSLRAEDTAVYYCARGGLYGSGSYLSDFDL |
| | | WGRGTLVTVSSASTKGPSVFPLAPCSRSTSESTAALGCL |
| | | VKDYFPEPVTVSWNSGALTSGVHTFPAVLQSSGLYSLSS |
| | | VVTVPSSSLGTKTYTCNVDHKPSNTKVDKRVESKYGPP |
| | | CPPCPAPEFLGGPSVFLFPPKPKDTLMISRTPEVTCVVVD |
| | | VSQEDPEVQFNWYVDGVEVHNAKTKPREEQFNSTYRV |
| | | VSVLTVLHQDWLNGKEYKCKVSNKGLPSSIEKTISKAK |
| | | GQPREPQVYTLPPSQEEMTKNQVSLTCLVKGFYPSDIAV |
| | | EWESNGQPENNYKTTPPVLDSDGSFFLYSRLTVDKSRW |
| | | QEGNVFSCSVMHEALHNHYTQKSLSLSLGX, |
| | | wherein X is K or absent |
| SEQ ID NO: 47 | 373.A | GAAGTGCAGCTGCTGGAATCTGGCGGCGGACTGGTG |
| | DNA | CAGCCTGGCGGCTCCCTGAGACTGTCTTGCGCCGCCT |
| | Heavy | CCGGCTTCACCTTCCACAGATACGCCATGTCCTGGGT |
| | Chain | CCGACAGGCCCCTGGCAAGGGCCTGGAGTGGGTGTC |
| | | CGCCATCTCCGGCTCCGGCATGAACACCTACTACGCC |
| | | GACTCCGTGAAGGGCCGGTTCACCATCTCCCGGGACA |
| | | ACTCCAAGAACACCCTGTACCTGCAGATGAACTCCCT |
| | | GCGGGCCGAGGACACCGCCGTGTACTACTGTGCCAG |
| | | AGGCGGCCTGTACGGCTCCGGCTCCTACCTGTCCGAC |
| | | TTCGACCTGTGGGGCAGAGGCACCCTGGTCACCGTGT |
| | | CCTCCGCCTCCACAAAGGGCCCCTCCGTGTTCCCTCT |
| | | GGCCCCTTGCTCCCGGTCCACCTCCGAGTCTACCGCC |
| | | GCTCTGGGCTGCCTCGTGAAGGACTACTTCCCCGAGC |
| | | CCGTGACCGTGTCCTGGAACTCTGGCGCCCTGACCTC |
| | | CGGCGTGCACACCTTCCCTGCCGTGCTGCAGTCCTCC |
| | | GGCCTGTACTCCCTGTCCAGCGTCGTGACCGTGCCCT |
| | | CCTCCAGCCTGGGCACCAAGACCTACACCTGTAACGT |
| | | GGACCACAAGCCCTCCAACACCAAAGTGGACAAGCG |
| | | GGTGGAATCTAAGTACGGCCCTCCCTGCCCTCCTTGC |
| | | CCTGCCCCTGAGTTCCTGGGCGGACCTTCCGTGTTCCT |
| | | GTTCCCTCCAAAGCCCAAGGACACCCTGATGATCTCC |
| | | CGGACCCCTGAAGTGACCTGCGTGGTGGTGGACGTGT |
| | | CCCAGGAAGATCCCGAAGTCCAGTTCAATTGGTACGT |
| | | GGACGGCGTGGAAGTGCACAACGCCAAGACCAAGCC |
| | | CAGAGAGGAACAGTTCAACTCCACCTACCGGGTGGT |
| | | GTCCGTGCTGACCGTGCTGCACCAGGACTGGCTGAAC |
| | | GGCAAAGAGTACAAGTGCAAAGTGTCCAACAAGGGC |
| | | CTGCCCTCCAGCATCGAAAAGACCATCTCCAAGGCCA |
| | | AGGGCCAGCCCCGCGAGCCCCAAGTGTACACCCTGCC |
| | | TCCCAGCCAGGAAGAGATGACCAAGAATCAAGTGTC |
| | | CCTGACTTGTCTGGTCAAGGGCTTCTACCCCTCCGAT |
| | | ATCGCCGTGGAGTGGGAGTCCAACGGCCAGCCCGAG |
| | | AACAACTACAAGACCACCCCTCCCGTGCTGGACTCCG |
| | | ACGGCTCCTTCTTCCTGTACTCTCGGCTGACCGTGGAC |
| | | AAGTCCCGGTGGCAGGAAGGCAACGTCTTCTCCTGCT |
| | | CCGTGATGCACGAGGCCCTGCACAACCACTACACCCA |
| | | GAAGTCCCTGTCCCTGTCTCTGGGC X₁X₂X₃, |
| | | wherein X₁ is A, X₂ is A, and X₃ is G; or X₁ is |
| | | absent, X₂ is absent, and X₃ is absent. |
| SEQ ID NO: 114 | 373.B | EVQLLESGGGLVQPGGSLRLSCAASGFTFHRYAMSWVR |
| | Heavy | QAPGKGLEWVSAISGSGMNTYYADSVKGRFTISRDNSK |
| | chain | NTLYLQMNSLRAEDTAVYYCARGGLYGSGSYLSDFDL |
| | | WGRGTLVTVSSASTKGPSVFPLAPCSRSTSESTAALGCL |
| | | VKDYFPEPVTVSWNSGALTSGVHTFPAVLQSSGLYSLSS |
| | | VVTVPSSSLGTKTYTCNVDHKPSNTKVDKRVESKYGPP |
| | | CPPCPAPEFEGGPSVFLFPPKPKDTLMISRTPEVTCVVVD |
| | | VSQEDPEVQFNWYVDGVEVHNAKTKPREEQFNSTYRV |
| | | VSVLTVLHQDWLNGKEYKCKVSNKGLPSSIEKTISKAK |
| | | GQPREPQVYTLPPSQEEMTKNQVSLTCLVKGFYPSDIAV |
| | | EWESNGQPENNYKTTPPVLDSDGSFFLYSRLTVDKSRW |
| | | QEGNVFSCSVMHEALHNHYTQKSLSLSLGX, |
| | | wherein X is K or absent |
| SEQ ID NO: 48 | LCDR1 | RASQSVGSNLA |
| (Combined) | | |
| SEQ ID NO: 49 | LCDR2 | GASTRAT |
| (Combined) | | |
| SEQ ID NO: 50 | LCDR3 | QQHNAFPYT |
| (Combined) | | |
| SEQ ID NO: 48 (Kabat) | LCDR1 | RASQSVGSNLA |
| SEQ ID NO: 49 (Kabat) | LCDR2 | GASTRAT |
| SEQ ID NO: 50 (Kabat) | LCDR3 | QQHNAFPYT |
| SEQ ID NO: 51 (Chothia) | LCDR1 | SQSVGSN |
| SEQ ID NO: 52 (Chothia) | LCDR2 | GAS |
| SEQ ID NO: 53 (Chothia) | LCDR3 | HNAFPY |
| SEQ ID NO: 54 (IMGT) | LCDR1 | QSVGSN |
| SEQ ID NO: 52 (IMGT) | LCDR2 | GAS |
| SEQ ID NO: 50 (IMGT) | LCDR3 | QQHNAFPYT |
| SEQ ID NO: 48 | LCDR1 | RASQSVGSNLA |
| (Alternative) | | |
| SEQ ID NO: 49 | LCDR2 | GASTRAT |
| (Alternative) | | |
| SEQ ID NO: 50 | LCDR3 | QQHNAFPYT |
| (Alternative) | | |
| SEQ ID NO: 55 | VL | EIVLTQSPATLSVSPGERATLSCRASQSVGSNLAWYQQK |
| | | PGQAPRLLIYGASTRATGIPARFSGSGSGTEFTLTISSLQS |
| | | EDFAVYYCQQHNAFPYTFGGGTKVEIK |
| SEQ ID NO: 56 | DNA VL | GAGATCGTGCTGACACAGTCCCCTGCCACCCTGTCTG |
| | | TGTCTCCCGGCGAGAGAGCCACCCTGAGCTGCAGAGC |
| | | CTCCCAGTCCGTGGGCTCCAACCTGGCCTGGTATCAG |
| | | CAGAAGCCCGGCCAGGCCCCTCGGCTGCTGATCTACG |
| | | GCGCCTCTACCAGAGCCACCGGCATCCCTGCCAGATT |
| | | CTCCGGCTCTGGCTCCGGCACCGAGTTCACCCTGACC |
| | | ATCTCCAGCCTGCAGTCCGAGGACTTCGCCGTGTACT |
| | | ACTGCCAGCAGCACAACGCCTTCCCTTACACCTTCGG |
| | | CGGAGGCACCAAAGTGGAAATCAAG |
| SEQ ID NO: 57 | Light | EIVLTQSPATLSVSPGERATLSCRASQSVGSNLAWYQQK |
| | Chain | PGQAPRLLIYGASTRATGIPARFSGSGSGTEFTLTISSLQS |
| | | EDFAVYYCQQHNAFPYTFGGGTKVEIKRTVAAPSVFIFP |
| | | PSDEQLKSGTASVVCLLNNFYPREAKVQWKVDNALQS |
| | | GNSQESVTEQDSKDSTYSLSSTLTLSKADYEKHKVYAC |
| | | EVTHQGLSSPVTKSFNRGEC |
| SEQ ID NO: 58 | DNA | GAGATCGTGCTGACACAGTCCCCTGCCACCCTGTCTG |
| | Light | TGTCTCCCGGCGAGAGAGCCACCCTGAGCTGCAGAGC |
| | Chain | CTCCCAGTCCGTGGGCTCCAACCTGGCCTGGTATCAG |
| | | CAGAAGCCCGGCCAGGCCCCTCGGCTGCTGATCTACG |
| | | GCGCCTCTACCAGAGCCACCGGCATCCCTGCCAGATT |
| | | CTCCGGCTCTGGCTCCGGCACCGAGTTCACCCTGACC |
| | | ATCTCCAGCCTGCAGTCCGAGGACTTCGCCGTGTACT |
| | | ACTGCCAGCAGCACAACGCCTTCCCTTACACCTTCGG |
| | | CGGAGGCACCAAAGTGGAAATCAAGCGTACGGTGGC |
| | | CGCTCCCTCCGTGTTCATCTTCCCACCCTCCGACGAGC |
| | | AGCTGAAGTCCGGCACCGCCTCCGTCGTGTGCCTGCT |
| | | GAACAACTTCTACCCTCGCGAGGCCAAAGTGCAGTGG |
| | | AAAGTGGACAACGCCCTGCAGTCCGGCAACTCCCAG |
| | | GAATCCGTCACCGAGCAGGACTCCAAGGACAGCACC |
| | | TACTCCCTGTCCTCCACCCTGACCCTGTCCAAGGCCG |
| | | ACTACGAGAAGCACAAAGTGTACGCCTGCGAAGTGA |
| | | CCCACCAGGGCCTGTCCAGCCCCGTGACCAAGTCCTT |
| | | CAACCGGGGCGAGTGC |
| 350 | | |
| SEQ ID NO: 59 | HCDR1 | GGSIERYYWS |
| (Combined) | | |
| SEQ ID NO: 60 | HCDR2 | YIYGRGSTNYNPSLKS |
| (Combined) | | |
| SEQ ID NO: 3 (Combined) | HCDR3 | ESQESPYNNWFDP |
| SEQ ID NO: 61 (Kabat) | HCDR1 | RYYWS |
| SEQ ID NO: 60 (Kabat) | HCDR2 | YIYGRGSTNYNPSLKS |
| SEQ ID NO: 3 (Kabat) | HCDR3 | ESQESPYNNWFDP |
| SEQ ID NO: 62 (Chothia) | HCDR1 | GGSIERY |
| SEQ ID NO: 63 (Chothia) | HCDR2 | YGRGS |
| SEQ ID NO: 3 (Chothia) | HCDR3 | ESQESPYNNWFDP |
| SEQ ID NO: 64 (IMGT) | HCDR1 | GGSIERYY |
| SEQ ID NO: 65 (IMGT) | HCDR2 | IYGRGST |
| SEQ ID NO: 9 (IMGT) | HCDR3 | ARESQESPYNNWFDP |
| SEQ ID NO: 197 | HCDR1 | GSIERYYWS |
| (Alternative) | | |
| SEQ ID NO: 60 | HCDR2 | YIYGRGSTNYNPSLKS |
| (Alternative) | | |
| SEQ ID NO: 9 | HCDR3 | ARESQESPYNNWFDP |
| (Alternative) | | |
| SEQ ID NO: 66 | VH | QVQLQESGPGLVKPSETLSLTCTVSGGSIERYYWSWIRQ |
| | | PPGKGLEWIGYIYGRGSTNYNPSLKSRVTISVDTSKNQF |
| | | SLKLSSVTAADTAVYYCARESQESPYNNWFDPWGQGT |
| | | LVTVSS |
| SEQ ID NO: 67 | DNA VH | CAAGTGCAGCTGCAGGAATCTGGCCCTGGCCTGGTCA |
| | | AGCCCTCCGAGACACTGTCCCTGACCTGCACCGTGTC |
| | | CGGCGGCTCCATCGAGCGGTACTACTGGTCCTGGATC |
| | | CGGCAGCCTCCCGGCAAGGGCCTGGAATGGATCGGC |
| | | TACATCTACGGCAGAGGCTCCACCAACTACAACCCCA |
| | | GCCTGAAGTCCAGAGTGACCATCTCCGTGGACACCTC |
| | | CAAGAACCAGTTCTCCCTGAAGCTGTCCTCCGTGACC |
| | | GCCGCTGACACCGCCGTGTACTACTGCGCCAGAGAGT |
| | | CCCAGGAATCCCCTTACAACAATTGGTTCGACCCCTG |
| | | GGGCCAGGGCACCCTGGTCACCGTGTCCTCT |
| SEQ ID NO: 68 | 350.A | QVQLQESGPGLVKPSETLSLTCTVSGGSIERYYWSWIRQ |
| | Heavy | PPGKGLEWIGYIYGRGSTNYNPSLKSRVTISVDTSKNQF |
| | Chain | SLKLSSVTAADTAVYYCARESQESPYNNWFDPWGQGT |
| | | LVTVSSASTKGPSVFPLAPCSRSTSESTAALGCLVKDYFP |
| | | EPVTVSWNSGALTSGVHTFPAVLQSSGLYSLSSVVTVPS |
| | | SSLGTKTYTCNVDHKPSNTKVDKRVESKYGPPCPPCPAP |
| | | EFLGGPSVFLFPPKPKDTLMISRTPEVTCVVVDVSQEDPE |
| | | VQFNWYVDGVEVHNAKTKPREEQFNSTYRVVSVLTVL |
| | | HQDWLNGKEYKCKVSNKGLPSSIEKTISKAKGQPREPQ |
| | | VYTLPPSQEEMTKNQVSLTCLVKGFYPSDIAVEWESNG |
| | | QPENNYKTTPPVLDSDGSFFLYSRLTVDKSRWQEGNVF |
| | | SCSVMHEALHNHYTQKSLSLSLGX, |
| | | wherein X is K or absent |
| SEQ ID NO: 69 | 350.A | CAAGTGCAGCTGCAGGAATCTGGCCCTGGCCTGGTCA |
| | DNA | AGCCCTCCGAGACACTGTCCCTGACCTGCACCGTGTC |
| | Heavy | CGGCGGCTCCATCGAGCGGTACTACTGGTCCTGGATC |
| | Chain | CGGCAGCCTCCCGGCAAGGGCCTGGAATGGATCGGC |
| | | TACATCTACGGCAGAGGCTCCACCAACTACAACCCCA |
| | | GCCTGAAGTCCAGAGTGACCATCTCCGTGGACACCTC |
| | | CAAGAACCAGTTCTCCCTGAAGCTGTCCTCCGTGACC |
| | | GCCGCTGACACCGCCGTGTACTACTGCGCCAGAGAGT |
| | | CCCAGGAATCCCCTTACAACAATTGGTTCGACCCCTG |
| | | GGGCCAGGGCACCCTGGTCACCGTGTCCTCTGCCTCC |
| | | ACCAAGGGCCCCTCCGTGTTCCCTCTGGCCCCTTGCTC |
| | | CCGGTCCACCTCCGAGTCTACCGCCGCTCTGGGCTGC |
| | | CTCGTGAAGGACTACTTCCCCGAGCCCGTGACCGTGT |
| | | CCTGGAACTCTGGCGCCCTGACCTCCGGCGTGCACAC |
| | | CTTCCCTGCCGTGCTGCAGTCCTCCGGCCTGTACTCCC |
| | | TGTCCAGCGTCGTGACCGTGCCCTCCTCCAGCCTGGG |
| | | CACCAAGACCTACACCTGTAACGTGGACCACAAGCCC |
| | | TCCAACACCAAAGTGGACAAGCGGGTGGAATCTAAG |
| | | TACGGCCCTCCCTGCCCTCCTTGCCCTGCCCCTGAGTT |
| | | CCTGGGCGGACCTTCCGTGTTCCTGTTCCCTCCAAAG |
| | | CCCAAGGACACCCTGATGATCTCCCGGACCCCTGAAG |
| | | TGACCTGCGTGGTGGTGGACGTGTCCCAGGAAGATCC |
| | | CGAAGTCCAGTTCAATTGGTACGTGGACGGCGTGGAA |
| | | GTGCACAACGCCAAGACCAAGCCCAGAGAGGAACAG |
| | | TTCAACTCCACCTACCGGGTGGTGTCCGTGCTGACCG |
| | | TGCTGCACCAGGACTGGCTGAACGGCAAAGAGTACA |
| | | AGTGCAAAGTGTCCAACAAGGGCCTGCCCTCCAGCAT |
| | | CGAAAAGACCATCTCCAAGGCCAAGGGCCAGCCCCG |
| | | CGAGCCCCAAGTGTACACCCTGCCTCCCAGCCAGGAA |
| | | GAGATGACCAAGAATCAAGTGTCCCTGACTTGTCTGG |
| | | TCAAGGGCTTCTACCCCTCCGATATCGCCGTGGAGTG |
| | | GGAGTCCAACGGCCAGCCCGAGAACAACTACAAGAC |
| | | CACCCCTCCCGTGCTGGACTCCGACGGCTCCTTCTTCC |
| | | TGTACTCTCGGCTGACCGTGGACAAGTCCCGGTGGCA |
| | | GGAAGGCAACGTCTTCTCCTGCTCCGTGATGCACGAG |
| | | GCCCTGCACAACCACTACACCCAGAAGTCCCTGTCCC |
| | | TGTCTCTGGGC X₁X₂X₃, |
| | | wherein X₁ is A, X₂ is A, and X₃ is G; or X₁ is |
| | | absent, X₂ is absent, and X₃ is absent. |
| SEQ ID NO: 115 | 350.B | QVQLQESGPGLVKPSETLSLTCTVSGGSIERYYWSWIRQ |
| | Heavy | PPGKGLEWIGYIYGRGSTNYNPSLKSRVTISVDTSKNQF |
| | chain | SLKLSSVTAADTAVYYCARESQESPYNNWFDPWGQGT |
| | | LVTVSSASTKGPSVFPLAPCSRSTSESTAALGCLVKDYFP |
| | | EPVTVSWNSGALTSGVHTFPAVLQSSGLYSLSSVVTVPS |
| | | SSLGTKTYTCNVDHKPSNTKVDKRVESKYGPPCPPCPAP |
| | | EFEGGPSVFLFPPKPKDTLMISRTPEVTCVVVDVSQEDPE |
| | | VQFNWYVDGVEVHNAKTKPREEQFNSTYRVVSVLTVL |
| | | HQDWLNGKEYKCKVSNKGLPSSIEKTISKAKGQPREPQ |
| | | VYTLPPSQEEMTKNQVSLTCLVKGFYPSDIAVEWESNG |
| | | QPENNYKTTPPVLDSDGSFFLYSRLTVDKSRWQEGNVF |
| | | SCSVMHEALHNHYTQKSLSLSLGX, |
| | | wherein X is K or absent |
| SEQ ID NO: 14 | LCDR1 | RASQGISSWLA |
| (Combined) | | |
| SEQ ID NO: 15 | LCDR2 | AASSLQS |
| (Combined) | | |
| SEQ ID NO: 16 | LCDR3 | QQGNSFPRT |
| (Combined) | | |
| SEQ ID NO: 14 (Kabat) | LCDR1 | RASQGISSWLA |
| SEQ ID NO: 15 (Kabat) | LCDR2 | AASSLQS |
| SEQ ID NO: 16 (Kabat) | LCDR3 | QQGNSFPRT |
| SEQ ID NO: 17 (Chothia) | LCDR1 | SQGISSW |
| SEQ ID NO: 18 (Chothia) | LCDR2 | AAS |
| SEQ ID NO: 19 (Chothia) | LCDR3 | GNSFPR |
| SEQ ID NO: 20 (IMGT) | LCDR1 | QGISSW |
| SEQ ID NO: 18 (IMGT) | LCDR2 | AAS |
| SEQ ID NO: 16 (IMGT) | LCDR3 | QQGNSFPRT |
| SEQ ID NO: 14 | LCDR1 | RASQGISSWLA |
| (Alternative) | | |
| SEQ ID NO: 15 | LCDR2 | AASSLQS |
| (Alternative) | | |
| SEQ ID NO: 16 | LCDR3 | QQGNSFPRT |
| (Alternative) | | |
| SEQ ID NO: 21 | VL | DIQMTQSPSSVSASVGDRVTITCRASQGISSWLAWYQQ |
| | | KPGKAPKLLIYAASSLQSGVPSRFSGSGSGTDFTLTISSL |
| | | QPEDFATYYCQQGNSFPRTFGGGTKVEIK |
| SEQ ID NO: 22 | DNA VL | GACATCCAGATGACCCAGAGCCCCTCCTCCGTGTCCG |
| | | CCTCCGTGGGCGACAGAGTGACCATCACCTGTCGGGC |
| | | CTCCCAGGGCATCTCCAGCTGGCTGGCCTGGTATCAG |
| | | CAGAAGCCCGGCAAGGCCCCTAAGCTGCTGATCTACG |
| | | CCGCCTCCAGCCTGCAGTCCGGCGTGCCCTCCAGATT |
| | | CTCCGGCTCTGGCTCCGGCACCGACTTCACCCTGACC |
| | | ATCAGCTCCCTGCAGCCCGAGGACTTCGCCACCTACT |
| | | ACTGCCAGCAGGGCAACTCCTTCCCTCGGACCTTCGG |
| | | CGGAGGCACCAAAGTGGAAATCAAG |
| SEQ ID NO: 23 | Light | DIQMTQSPSSVSASVGDRVTITCRASQGISSWLAWYQQ |
| | Chain | KPGKAPKLLIYAASSLQSGVPSRFSGSGSGTDFTLTISSL |
| | | QPEDFATYYCQQGNSFPRTFGGGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYA |
| | | CEVTHQGLSSPVTKSFNRGEC |
| SEQ ID NO: 24 | DNA | GACATCCAGATGACCCAGAGCCCCTCCTCCGTGTCCG |
| | Light | CCTCCGTGGGCGACAGAGTGACCATCACCTGTCGGGC |
| | Chain | CTCCCAGGGCATCTCCAGCTGGCTGGCCTGGTATCAG |
| | | CAGAAGCCCGGCAAGGCCCCTAAGCTGCTGATCTACG |
| | | CCGCCTCCAGCCTGCAGTCCGGCGTGCCCTCCAGATT |
| | | CTCCGGCTCTGGCTCCGGCACCGACTTCACCCTGACC |
| | | ATCAGCTCCCTGCAGCCCGAGGACTTCGCCACCTACT |
| | | ACTGCCAGCAGGGCAACTCCTTCCCTCGGACCTTCGG |
| | | CGGAGGCACCAAAGTGGAAATCAAGCGTACGGTGGC |
| | | CGCTCCCTCCGTGTTCATCTTCCCACCCTCCGACGAGC |
| | | AGCTGAAGTCCGGCACCGCCTCCGTCGTGTGCCTGCT |
| | | GAACAACTTCTACCCTCGCGAGGCCAAAGTGCAGTGG |
| | | AAAGTGGACAACGCCCTGCAGTCCGGCAACTCCCAG |
| | | GAATCCGTCACCGAGCAGGACTCCAAGGACAGCACC |
| | | TACTCCCTGTCCTCCACCCTGACCCTGTCCAAGGCCG |
| | | ACTACGAGAAGCACAAAGTGTACGCCTGCGAAGTGA |
| | | CCCACCAGGGCCTGTCCAGCCCCGTGACCAAGTCCTT |
| | | CAACCGGGGCGAGTGC |
| 374 | | |
| SEQ ID NO: 70 | HCDR1 | GFTFSYNAMS |
| (Combined) | | |
| SEQ ID NO: 71 | HCDR2 | SISGTGGSTYYADSVKG |
| (Combined) | | |
| SEQ ID NO: 37 | HCDR3 | GGLYGSGSYLSDFDL |
| (Combined) | | |
| SEQ ID NO: 72 (Kabat) | HCDR1 | YNAMS |
| SEQ ID NO: 71 (Kabat) | HCDR2 | SISGTGGSTYYADSVKG |
| SEQ ID NO: 37 (Kabat) | HCDR3 | GGLYGSGSYLSDFDL |
| SEQ ID NO: 73 (Chothia) | HCDR1 | GFTFSYN |
| SEQ ID NO: 74 (Chothia) | HCDR2 | SGTGGS |
| SEQ ID NO: 37 (Chothia) | HCDR3 | GGLYGSGSYLSDFDL |
| SEQ ID NO: 75 (IMGT) | HCDR1 | GFTFSYNA |
| SEQ ID NO: 76 (IMGT) | HCDR2 | ISGTGGST |
| SEQ ID NO: 43 (IMGT) | HCDR3 | ARGGLYGSGSYLSDFDL |
| SEQ ID NO: 191 | HCDR1 | FTFSYNAMS |
| (Alternative) | | |
| SEQ ID NO: 71 | HCDR2 | SISGTGGSTYYADSVKG |
| (Alternative) | | |
| SEQ ID NO: 43 | HCDR3 | ARGGLYGSGSYLSDFDL |
| (Alternative) | | |
| SEQ ID NO: 77 | VH | EVQLLESGGGLVQPGGSLRLSCAASGFTFSYNAMSWVR |
| | | QAPGKGLEWVSSISGTGGSTYYADSVKGRFTISRDNSKN |
| | | TLYLQMNSLRAEDTAVYYCARGGLYGSGSYLSDFDLW |
| | | GRGTLVTVSS |
| SEQ ID NO: 78 | DNA VH | GAAGTGCAGCTGCTGGAATCTGGCGGCGGACTGGTG |
| | | CAGCCTGGCGGCTCCCTGAGACTGTCTTGCGCCGCCT |
| | | CCGGCTTCACCTTCTCCTACAACGCCATGTCCTGGGTC |
| | | CGACAGGCCCCTGGCAAGGGCCTGGAGTGGGTGTCCT |
| | | CCATCTCCGGCACCGGCGGCTCCACCTACTACGCCGA |
| | | CTCTGTGAAGGGCCGGTTCACCATCTCCCGGGACAAC |
| | | TCCAAGAACACCCTGTACCTGCAGATGAACTCCCTGC |
| | | GGGCCGAGGACACCGCCGTGTACTACTGTGCCAGAG |
| | | GCGGCCTGTACGGCTCCGGCTCCTACCTGTCCGACTT |
| | | CGACCTGTGGGGCAGAGGCACCCTGGTCACCGTGTCC |
| | | TCC |
| SEQ ID NO: 79 | 374.A | EVQLLESGGGLVQPGGSLRLSCAASGFTFSYNAMSWVR |
| | Heavy | QAPGKGLEWVSSISGTGGSTYYADSVKGRFTISRDNSKN |
| | Chain | TLYLQMNSLRAEDTAVYYCARGGLYGSGSYLSDFDLW |
| | | GRGTLVTVSSASTKGPSVFPLAPCSRSTSESTAALGCLV |
| | | KDYFPEPVTVSWNSGALTSGVHTFPAVLQSSGLYSLSSV |
| | | VTVPSSSLGTKTYTCNVDHKPSNTKVDKRVESKYGPPC |
| | | PPCPAPEFLGGPSVFLFPPKPKDTLMISRTPEVTCVVVDV |
| | | SQEDPEVQFNWYVDGVEVHNAKTKPREEQFNSTYRVV |
| | | SVLTVLHQDWLNGKEYKCKVSNKGLPSSIEKTISKAKG |
| | | QPREPQVYTLPPSQEEMTKNQVSLTCLVKGFYPSDIAVE |
| | | WESNGQPENNYKTTPPVLDSDGSFFLYSRLTVDKSRWQ |
| | | EGNVFSCSVMHEALHNHYTQKSLSLSLGX, |
| | | wherein X is K or absent |
| SEQ ID NO: 80 | 374.A | GAAGTGCAGCTGCTGGAATCTGGCGGCGGACTGGTG |
| | DNA | CAGCCTGGCGGCTCCCTGAGACTGTCTTGCGCCGCCT |
| | Heavy | CCGGCTTCACCTTCTCCTACAACGCCATGTCCTGGGTC |
| | Chain | CGACAGGCCCCTGGCAAGGGCCTGGAGTGGGTGTCCT |
| | | CCATCTCCGGCACCGGCGGCTCCACCTACTACGCCGA |
| | | CTCTGTGAAGGGCCGGTTCACCATCTCCCGGGACAAC |
| | | TCCAAGAACACCCTGTACCTGCAGATGAACTCCCTGC |
| | | GGGCCGAGGACACCGCCGTGTACTACTGTGCCAGAG |
| | | GCGGCCTGTACGGCTCCGGCTCCTACCTGTCCGACTT |
| | | CGACCTGTGGGGCAGAGGCACCCTGGTCACCGTGTCC |
| | | TCCGCCTCCACAAAGGGCCCCTCCGTGTTCCCTCTGG |
| | | CCCCTTGCTCCCGGTCCACCTCCGAGTCTACCGCCGCT |
| | | CTGGGCTGCCTCGTGAAGGACTACTTCCCCGAGCCCG |
| | | TGACCGTGTCCTGGAACTCTGGCGCCCTGACCTCCGG |
| | | CGTGCACACCTTCCCTGCCGTGCTGCAGTCCTCCGGC |
| | | CTGTACTCCCTGTCCAGCGTCGTGACCGTGCCCTCCTC |
| | | CAGCCTGGGCACCAAGACCTACACCTGTAACGTGGAC |
| | | CACAAGCCCTCCAACACCAAAGTGGACAAGCGGGTG |
| | | GAATCTAAGTACGGCCCTCCCTGCCCTCCTTGCCCTG |
| | | CCCCTGAGTTCCTGGGCGGACCTTCCGTGTTCCTGTTC |
| | | CCTCCAAAGCCCAAGGACACCCTGATGATCTCCCGGA |
| | | CCCCTGAAGTGACCTGCGTGGTGGTGGACGTGTCCCA |
| | | GGAAGATCCCGAAGTCCAGTTCAATTGGTACGTGGAC |
| | | GGCGTGGAAGTGCACAACGCCAAGACCAAGCCCAGA |
| | | GAGGAACAGTTCAACTCCACCTACCGGGTGGTGTCCG |
| | | TGCTGACCGTGCTGCACCAGGACTGGCTGAACGGCAA |
| | | AGAGTACAAGTGCAAAGTGTCCAACAAGGGCCTGCC |
| | | CTCCAGCATCGAAAAGACCATCTCCAAGGCCAAGGG |
| | | CCAGCCCCGCGAGCCCCAAGTGTACACCCTGCCTCCC |
| | | AGCCAGGAAGAGATGACCAAGAATCAAGTGTCCCTG |
| | | ACTTGTCTGGTCAAGGGCTTCTACCCCTCCGATATCG |
| | | CCGTGGAGTGGGAGTCCAACGGCCAGCCCGAGAACA |
| | | ACTACAAGACCACCCCTCCCGTGCTGGACTCCGACGG |
| | | CTCCTTCTTCCTGTACTCTCGGCTGACCGTGGACAAGT |
| | | CCCGGTGGCAGGAAGGCAACGTCTTCTCCTGCTCCGT |
| | | GATGCACGAGGCCCTGCACAACCACTACACCCAGAA |
| | | GTCCCTGTCCCTGTCTCTGGGC X₁X₂X₃, |
| | | wherein X₁ is A, X₂ is A, and X₃ is G; or X₁ is |
| | | absent, X₂ is absent, and X₃ is absent. |
| SEQ ID NO: 116 | 374.B | EVQLLESGGGLVQPGGSLRLSCAASGFTFSYNAMSWVR |
| | Heavy | QAPGKGLEWVSSISGTGGSTYYADSVKGRFTISRDNSKN |
| | chain | TLYLQMNSLRAEDTAVYYCARGGLYGSGSYLSDFDLW |
| | | GRGTLVTVSSASTKGPSVFPLAPCSRSTSESTAALGCLV |
| | | KDYFPEPVTVSWNSGALTSGVHTFPAVLQSSGLYSLSSV |
| | | VTVPSSSLGTKTYTCNVDHKPSNTKVDKRVESKYGPPC |
| | | PPCPAPEFEGGPSVFLFPPKPKDTLMISRTPEVTCVVVDV |
| | | SQEDPEVQFNWYVDGVEVHNAKTKPREEQFNSTYRVV |
| | | SVLTVLHQDWLNGKEYKCKVSNKGLPSSIEKTISKAKG |
| | | QPREPQVYTLPPSQEEMTKNQVSLTCLVKGFYPSDIAVE |
| | | WESNGQPENNYKTTPPVLDSDGSFFLYSRLTVDKSRWQ |
| | | EGNVFSCSVMHEALHNHYTQKSLSLSLGX, |
| | | wherein X is K or absent |
| SEQ ID NO: 48 | LCDR1 | RASQSVGSNLA |
| (Combined) | | |
| SEQ ID NO: 49 | LCDR2 | GASTRAT |
| (Combined) | | |
| SEQ ID NO: 50 | LCDR3 | QQHNAFPYT |
| (Combined) | | |
| SEQ ID NO: 48 (Kabat) | LCDR1 | RASQSVGSNLA |
| SEQ ID NO: 49 (Kabat) | LCDR2 | GASTRAT |
| SEQ ID NO: 50 (Kabat) | LCDR3 | QQHNAFPYT |
| SEQ ID NO: 51 (Chothia) | LCDR1 | SQSVGSN |
| SEQ ID NO: 52 (Chothia) | LCDR2 | GAS |
| SEQ ID NO: 53 (Chothia) | LCDR3 | HNAFPY |
| SEQ ID NO: 54 (IMGT) | LCDR1 | QSVGSN |
| SEQ ID NO: 52 (IMGT) | LCDR2 | GAS |
| SEQ ID NO: 50 (IMGT) | LCDR3 | QQHNAFPYT |
| SEQ ID NO: 48 | LCDR1 | RASQSVGSNLA |
| (Alternative) | | |
| SEQ ID NO: 49 | LCDR2 | GASTRAT |
| (Alternative) | | |
| SEQ ID NO: 50 | LCDR3 | QQHNAFPYT |
| (Alternative) | | |
| SEQ ID NO: 55 | VL | EIVLTQSPATLSVSPGERATLSCRASQSVGSNLAWYQQK |
| | | PGQAPRLLIYGASTRATGIPARFSGSGSGTEFTLTISSLQS |
| | | EDFAVYYCQQHNAFPYTFGGGTKVEIK |
| SEQ ID NO: 56 | DNA VL | GAGATCGTGCTGACACAGTCCCCTGCCACCCTGTCTG |
| | | TGTCTCCCGGCGAGAGAGCCACCCTGAGCTGCAGAGC |
| | | CTCCCAGTCCGTGGGCTCCAACCTGGCCTGGTATCAG |
| | | CAGAAGCCCGGCCAGGCCCCTCGGCTGCTGATCTACG |
| | | GCGCCTCTACCAGAGCCACCGGCATCCCTGCCAGATT |
| | | CTCCGGCTCTGGCTCCGGCACCGAGTTCACCCTGACC |
| | | ATCTCCAGCCTGCAGTCCGAGGACTTCGCCGTGTACT |
| | | ACTGCCAGCAGCACAACGCCTTCCCTTACACCTTCGG |
| | | CGGAGGCACCAAAGTGGAAATCAAG |
| SEQ ID NO: 57 | Light | EIVLTQSPATLSVSPGERATLSCRASQSVGSNLAWYQQK |
| | Chain | PGQAPRLLIYGASTRATGIPARFSGSGSGTEFTLTISSLQS |
| | | EDFAVYYCQQHNAFPYTFGGGTKVEIKRTVAAPSVFIFP |
| | | PSDEQLKSGTASVVCLLNNFYPREAKVQWKVDNALQS |
| | | GNSQESVTEQDSKDSTYSLSSTLTLSKADYEKHKVYAC |
| | | EVTHQGLSSPVTKSFNRGEC |
| SEQ ID NO: 58 | DNA | GAGATCGTGCTGACACAGTCCCCTGCCACCCTGTCTG |
| | Light | TGTCTCCCGGCGAGAGAGCCACCCTGAGCTGCAGAGC |
| | Chain | CTCCCAGTCCGTGGGCTCCAACCTGGCCTGGTATCAG |
| | | CAGAAGCCCGGCCAGGCCCCTCGGCTGCTGATCTACG |
| | | GCGCCTCTACCAGAGCCACCGGCATCCCTGCCAGATT |
| | | CTCCGGCTCTGGCTCCGGCACCGAGTTCACCCTGACC |
| | | ATCTCCAGCCTGCAGTCCGAGGACTTCGCCGTGTACT |
| | | ACTGCCAGCAGCACAACGCCTTCCCTTACACCTTCGG |
| | | CGGAGGCACCAAAGTGGAAATCAAGCGTACGGTGGC |
| | | CGCTCCCTCCGTGTTCATCTTCCCACCCTCCGACGAGC |
| | | AGCTGAAGTCCGGCACCGCCTCCGTCGTGTGCCTGCT |
| | | GAACAACTTCTACCCTCGCGAGGCCAAAGTGCAGTGG |
| | | AAAGTGGACAACGCCCTGCAGTCCGGCAACTCCCAG |
| | | GAATCCGTCACCGAGCAGGACTCCAAGGACAGCACC |
| | | TACTCCCTGTCCTCCACCCTGACCCTGTCCAAGGCCG |
| | | ACTACGAGAAGCACAAAGTGTACGCCTGCGAAGTGA |
| | | CCCACCAGGGCCTGTCCAGCCCCGTGACCAAGTCCTT |
| | | CAACCGGGGCGAGTGC |
| 379 | | |
| SEQ ID NO: 81 | HCDR1 | GFTFSRYAMS |
| (Combined) | | |
| SEQ ID NO: 71 | HCDR2 | SISGTGGSTYYADSVKG |
| (Combined) | | |
| SEQ ID NO: 37 | HCDR3 | GGLYGSGSYLSDFDL |
| (Combined) | | |
| SEQ ID NO: 38 (Kabat) | HCDR1 | RYAMS |
| SEQ ID NO: 71 (Kabat) | HCDR2 | SISGTGGSTYYADSVKG |
| SEQ ID NO: 37 (Kabat) | HCDR3 | GGLYGSGSYLSDFDL |
| SEQ ID NO: 82 (Chothia) | HCDR1 | GFTFSRY |
| SEQ ID NO: 74 (Chothia) | HCDR2 | SGTGGS |
| SEQ ID NO: 37 (Chothia) | HCDR3 | GGLYGSGSYLSDFDL |
| SEQ ID NO: 83 (IMGT) | HCDR1 | GFTFSRYA |
| SEQ ID NO: 76 (IMGT) | HCDR2 | ISGTGGST |
| SEQ ID NO: 43 (IMGT) | HCDR3 | ARGGLYGSGSYLSDFDL |
| SEQ ID NO: 192 | HCDR1 | FTFSRYAMS |
| (Alternative) | | |
| SEQ ID NO: 71 | HCDR2 | SISGTGGSTYYADSVKG |
| (Alternative) | | |
| SEQ ID NO: 43 | HCDR3 | ARGGLYGSGSYLSDFDL |
| (Alternative) | | |
| SEQ ID NO: 84 | VH | EVQLLESGGGLVQPGGSLRLSCAASGFTFSRYAMSWVR |
| | | QAPGKGLEWVSSISGTGGSTYYADSVKGRFTISRDNSKN |
| | | TLYLQMNSLRAEDTAVYYCARGGLYGSGSYLSDFDLW |
| | | GRGTLVTVSS |
| SEQ ID NO: 85 | DNA VH | GAAGTGCAGCTGCTGGAATCTGGCGGCGGACTGGTG |
| | | CAGCCTGGCGGCTCCCTGAGACTGTCTTGCGCCGCCT |
| | | CCGGCTTCACCTTCTCCAGATACGCCATGTCCTGGGT |
| | | CCGACAGGCCCCTGGCAAGGGCCTGGAGTGGGTGTC |
| | | CTCCATCTCCGGCACCGGCGGCTCCACCTACTACGCC |
| | | GACTCTGTGAAGGGCCGGTTCACCATCTCCCGGGACA |
| | | ACTCCAAGAACACCCTGTACCTGCAGATGAACTCCCT |
| | | GCGGGCCGAGGACACCGCCGTGTACTACTGTGCCAG |
| | | AGGCGGCCTGTACGGCTCCGGCTCCTACCTGTCCGAC |
| | | TTCGACCTGTGGGGCAGAGGCACCCTGGTCACCGTGT |
| | | CCTCC |
| SEQ ID NO: 86 | 379.A | EVQLLESGGGLVQPGGSLRLSCAASGFTFSRYAMSWVR |
| | Heavy | QAPGKGLEWVSSISGTGGSTYYADSVKGRFTISRDNSKN |
| | Chain | TLYLQMNSLRAEDTAVYYCARGGLYGSGSYLSDFDLW |
| | | GRGTLVTVSSASTKGPSVFPLAPCSRSTSESTAALGCLV |
| | | KDYFPEPVTVSWNSGALTSGVHTFPAVLQSSGLYSLSSV |
| | | VTVPSSSLGTKTYTCNVDHKPSNTKVDKRVESKYGPPC |
| | | PPCPAPEFLGGPSVFLFPPKPKDTLMISRTPEVTCVVVDV |
| | | SQEDPEVQFNWYVDGVEVHNAKTKPREEQFNSTYRVV |
| | | SVLTVLHQDWLNGKEYKCKVSNKGLPSSIEKTISKAKG |
| | | QPREPQVYTLPPSQEEMTKNQVSLTCLVKGFYPSDIAVE |
| | | WESNGQPENNYKTTPPVLDSDGSFFLYSRLTVDKSRWQ |
| | | EGNVFSCSVMHEALHNHYTQKSLSLSLGX, |
| | | wherein X is K or absent |
| SEQ ID NO: 87 | 379.A | GAAGTGCAGCTGCTGGAATCTGGCGGCGGACTGGTG |
| | DNA | CAGCCTGGCGGCTCCCTGAGACTGTCTTGCGCCGCCT |
| | Heavy | CCGGCTTCACCTTCTCCAGATACGCCATGTCCTGGGT |
| | Chain | CCGACAGGCCCCTGGCAAGGGCCTGGAGTGGGTGTC |
| | | CTCCATCTCCGGCACCGGCGGCTCCACCTACTACGCC |
| | | GACTCTGTGAAGGGCCGGTTCACCATCTCCCGGGACA |
| | | ACTCCAAGAACACCCTGTACCTGCAGATGAACTCCCT |
| | | GCGGGCCGAGGACACCGCCGTGTACTACTGTGCCAG |
| | | AGGCGGCCTGTACGGCTCCGGCTCCTACCTGTCCGAC |
| | | TTCGACCTGTGGGGCAGAGGCACCCTGGTCACCGTGT |
| | | CCTCCGCCTCCACAAAGGGCCCCTCCGTGTTCCCTCT |
| | | GGCCCCTTGCTCCCGGTCCACCTCCGAGTCTACCGCC |
| | | GCTCTGGGCTGCCTCGTGAAGGACTACTTCCCCGAGC |
| | | CCGTGACCGTGTCCTGGAACTCTGGCGCCCTGACCTC |
| | | CGGCGTGCACACCTTCCCTGCCGTGCTGCAGTCCTCC |
| | | GGCCTGTACTCCCTGTCCAGCGTCGTGACCGTGCCCT |
| | | CCTCCAGCCTGGGCACCAAGACCTACACCTGTAACGT |
| | | GGACCACAAGCCCTCCAACACCAAAGTGGACAAGCG |
| | | GGTGGAATCTAAGTACGGCCCTCCCTGCCCTCCTTGC |
| | | CCTGCCCCTGAGTTCCTGGGCGGACCTTCCGTGTTCCT |
| | | GTTCCCTCCAAAGCCCAAGGACACCCTGATGATCTCC |
| | | CGGACCCCTGAAGTGACCTGCGTGGTGGTGGACGTGT |
| | | CCCAGGAAGATCCCGAAGTCCAGTTCAATTGGTACGT |
| | | GGACGGCGTGGAAGTGCACAACGCCAAGACCAAGCC |
| | | CAGAGAGGAACAGTTCAACTCCACCTACCGGGTGGT |
| | | GTCCGTGCTGACCGTGCTGCACCAGGACTGGCTGAAC |
| | | GGCAAAGAGTACAAGTGCAAAGTGTCCAACAAGGGC |
| | | CTGCCCTCCAGCATCGAAAAGACCATCTCCAAGGCCA |
| | | AGGGCCAGCCCCGCGAGCCCCAAGTGTACACCCTGCC |
| | | TCCCAGCCAGGAAGAGATGACCAAGAATCAAGTGTC |
| | | CCTGACTTGTCTGGTCAAGGGCTTCTACCCCTCCGAT |
| | | ATCGCCGTGGAGTGGGAGTCCAACGGCCAGCCCGAG |
| | | AACAACTACAAGACCACCCCTCCCGTGCTGGACTCCG |
| | | ACGGCTCCTTCTTCCTGTACTCTCGGCTGACCGTGGAC |
| | | AAGTCCCGGTGGCAGGAAGGCAACGTCTTCTCCTGCT |
| | | CCGTGATGCACGAGGCCCTGCACAACCACTACACCCA |
| | | GAAGTCCCTGTCCCTGTCTCTGGGC X₁X₂X₃, |
| | | wherein X₁ is A, X₂ is A, and X₃ is G; or X₁ is |
| | | absent, X₂ is absent, and X₃ is absent. |
| SEQ ID NO: 117 | 379.B | EVQLLESGGGLVQPGGSLRLSCAASGFTFSRYAMSWVR |
| | Heavy | QAPGKGLEWVSSISGTGGSTYYADSVKGRFTISRDNSKN |
| | chain | TLYLQMNSLRAEDTAVYYCARGGLYGSGSYLSDFDLW |
| | | GRGTLVTVSSASTKGPSVFPLAPCSRSTSESTAALGCLV |
| | | KDYFPEPVTVSWNSGALTSGVHTFPAVLQSSGLYSLSSV |
| | | VTVPSSSLGTKTYTCNVDHKPSNTKVDKRVESKYGPPC |
| | | PPCPAPEFEGGPSVFLFPPKPKDTLMISRTPEVTCVVVDV |
| | | SQEDPEVQFNWYVDGVEVHNAKTKPREEQFNSTYRVV |
| | | SVLTVLHQDWLNGKEYKCKVSNKGLPSSIEKTISKAKG |
| | | QPREPQVYTLPPSQEEMTKNQVSLTCLVKGFYPSDIAVE |
| | | WESNGQPENNYKTTPPVLDSDGSFFLYSRLTVDKSRWQ |
| | | EGNVFSCSVMHEALHNHYTQKSLSLSLGX, |
| | | wherein X is K or absent |
| SEQ ID NO: 48 | LCDR1 | RASQSVGSNLA |
| (Combined) | | |
| SEQ ID NO: 49 | LCDR2 | GASTRAT |
| (Combined) | | |
| SEQ ID NO: 50 | LCDR3 | QQHNAFPYT |
| (Combined) | | |
| SEQ ID NO: 48 (Kabat) | LCDR1 | RASQSVGSNLA |
| SEQ ID NO: 49 (Kabat) | LCDR2 | GASTRAT |
| SEQ ID NO: 50 (Kabat) | LCDR3 | QQHNAFPYT |
| SEQ ID NO: 51 (Chothia) | LCDR1 | SQSVGSN |
| SEQ ID NO: 52 (Chothia) | LCDR2 | GAS |
| SEQ ID NO: 53 (Chothia) | LCDR3 | HNAFPY |
| SEQ ID NO: 54 (IMGT) | LCDR1 | QSVGSN |
| SEQ ID NO: 52 (IMGT) | LCDR2 | GAS |
| SEQ ID NO: 50 (IMGT) | LCDR3 | QQHNAFPYT |
| SEQ ID NO: 48 | LCDR1 | RASQSVGSNLA |
| (Alternative) | | |
| SEQ ID NO: 49 | LCDR2 | GASTRAT |
| (Alternative) | | |
| SEQ ID NO: 50 | LCDR3 | QQHNAFPYT |
| (Alternative) | | |
| SEQ ID NO: 55 | VL | EIVLTQSPATLSVSPGERATLSCRASQSVGSNLAWYQQK |
| | | PGQAPRLLIYGASTRATGIPARFSGSGSGTEFTLTISSLQS |
| | | EDFAVYYCQQHNAFPYTFGGGTKVEIK |
| SEQ ID NO: 56 | DNA VL | GAGATCGTGCTGACACAGTCCCCTGCCACCCTGTCTG |
| | | TGTCTCCCGGCGAGAGAGCCACCCTGAGCTGCAGAGC |
| | | CTCCCAGTCCGTGGGCTCCAACCTGGCCTGGTATCAG |
| | | CAGAAGCCCGGCCAGGCCCCTCGGCTGCTGATCTACG |
| | | GCGCCTCTACCAGAGCCACCGGCATCCCTGCCAGATT |
| | | CTCCGGCTCTGGCTCCGGCACCGAGTTCACCCTGACC |
| | | ATCTCCAGCCTGCAGTCCGAGGACTTCGCCGTGTACT |
| | | ACTGCCAGCAGCACAACGCCTTCCCTTACACCTTCGG |
| | | CGGAGGCACCAAAGTGGAAATCAAG |
| SEQ ID NO: 57 | Light | EIVLTQSPATLSVSPGERATLSCRASQSVGSNLAWYQQK |
| | Chain | PGQAPRLLIYGASTRATGIPARFSGSGSGTEFTLTISSLQS |
| | | EDFAVYYCQQHNAFPYTFGGGTKVEIKRTVAAPSVFIFP |
| | | PSDEQLKSGTASVVCLLNNFYPREAKVQWKVDNALQS |
| | | GNSQESVTEQDSKDSTYSLSSTLTLSKADYEKHKVYAC |
| | | EVTHQGLSSPVTKSFNRGEC |
| SEQ ID NO: 58 | DNA | GAGATCGTGCTGACACAGTCCCCTGCCACCCTGTCTG |
| | Light | TGTCTCCCGGCGAGAGAGCCACCCTGAGCTGCAGAGC |
| | Chain | CTCCCAGTCCGTGGGCTCCAACCTGGCCTGGTATCAG |
| | | CAGAAGCCCGGCCAGGCCCCTCGGCTGCTGATCTACG |
| | | GCGCCTCTACCAGAGCCACCGGCATCCCTGCCAGATT |
| | | CTCCGGCTCTGGCTCCGGCACCGAGTTCACCCTGACC |
| | | ATCTCCAGCCTGCAGTCCGAGGACTTCGCCGTGTACT |
| | | ACTGCCAGCAGCACAACGCCTTCCCTTACACCTTCGG |
| | | CGGAGGCACCAAAGTGGAAATCAAGCGTACGGTGGC |
| | | CGCTCCCTCCGTGTTCATCTTCCCACCCTCCGACGAGC |
| | | AGCTGAAGTCCGGCACCGCCTCCGTCGTGTGCCTGCT |
| | | GAACAACTTCTACCCTCGCGAGGCCAAAGTGCAGTGG |
| | | AAAGTGGACAACGCCCTGCAGTCCGGCAACTCCCAG |
| | | GAATCCGTCACCGAGCAGGACTCCAAGGACAGCACC |
| | | TACTCCCTGTCCTCCACCCTGACCCTGTCCAAGGCCG |
| | | ACTACGAGAAGCACAAAGTGTACGCCTGCGAAGTGA |
| | | CCCACCAGGGCCTGTCCAGCCCCGTGACCAAGTCCTT |
| | | CAACCGGGGCGAGTGC |
| 376 | | |
| SEQ ID NO: 135 | HCDR1 | GFTFRSYAMS |
| (Combined) | | |
| SEQ ID NO: 136 | HCDR2 | AITGSGGLTYYADSVKG |
| (Combined) | | |
| SEQ ID NO: 37 | HCDR3 | GGLYGSGSYLSDFDL |
| (Combined) | | |
| SEQ ID NO: 137 (Kabat) | HCDR1 | SYAMS |
| SEQ ID NO: 136 (Kabat) | HCDR2 | AITGSGGLTYYADSVKG |
| SEQ ID NO: 37 (Kabat) | HCDR3 | GGLYGSGSYLSDFDL |
| SEQ ID NO: 138 (Chothia) | HCDR1 | GFTFRSY |
| SEQ ID NO: 139 (Chothia) | HCDR2 | TGSGGL |
| SEQ ID NO: 37 (Chothia) | HCDR3 | GGLYGSGSYLSDFDL |
| SEQ ID NO: 140 (IMGT) | HCDR1 | GFTFRSYA |
| SEQ ID NO: 141 (IMGT) | HCDR2 | ITGSGGLT |
| SEQ ID NO: 43 (IMGT) | HCDR3 | ARGGLYGSGSYLSDFDL |
| SEQ ID NO: 193 | HCDR1 | FTFRSYAMS |
| (Alternative) | | |
| SEQ ID NO: 136 | HCDR2 | AITGSGGLTYYADSVKG |
| (Alternative) | | |
| SEQ ID NO: 43 | HCDR3 | ARGGLYGSGSYLSDFDL |
| (Alternative) | | |
| SEQ ID NO: 142 | VH | EVQLLESGGGLVQPGGSLRLSCAASGFTFRSYAMSWVR |
| | | QAPGKGLEWVSAITGSGGLTYYADSVKGRFTISRDNSK |
| | | NTLYLQMNSLRAEDTAVYYCARGGLYGSGSYLSDFDL |
| | | WGRGTLVTVSS |
| SEQ ID NO: 143 | DNA VH | GAGGTGCAGCTGTTGGAGTCTGGGGGAGGCTTGGTAC |
| | | AGCCTGGGGGGTCCCTGAGACTCTCCTGTGCAGCCTC |
| | | TGGATTCACCTTTCGTAGCTATGCCATGAGCTGGGTC |
| | | CGCCAGGCTCCAGGGAAGGGGCTGGAGTGGGTCTCA |
| | | GCTATTACGGGAAGTGGTGGTTTGACATACTACGCAG |
| | | ACTCCGTGAAGGGCCGGTTCACCATCTCCAGAGACAA |
| | | TTCCAAGAACACGCTGTATCTGCAAATGAACAGCCTG |
| | | AGAGCCGAGGACACGGCGGTGTACTACTGCGCCAGA |
| | | GGTGGATTGTACGGAAGCGGAAGCTACTTGAGTGACT |
| | | TCGACCTATGGGGGAGAGGTACCTTGGTCACCGTCTC |
| | | CTCA |
| SEQ ID NO: 48 | LCDR1 | RASQSVGSNLA |
| (Combined) | | |
| SEQ ID NO: 49 | LCDR2 | GASTRAT |
| (Combined) | | |
| SEQ ID NO: 50 | LCDR3 | QQHNAFPYT |
| (Combined) | | |
| SEQ ID NO: 48 (Kabat) | LCDR1 | RASQSVGSNLA |
| SEQ ID NO: 49 (Kabat) | LCDR2 | GASTRAT |
| SEQ ID NO: 50 (Kabat) | LCDR3 | QQHNAFPYT |
| SEQ ID NO: 51 (Chothia) | LCDR1 | SQSVGSN |
| SEQ ID NO: 52 (Chothia) | LCDR2 | GAS |
| SEQ ID NO: 53 (Chothia) | LCDR3 | HNAFPY |
| SEQ ID NO: 54 (IMGT) | LCDR1 | QSVGSN |
| SEQ ID NO: 52 (IMGT) | LCDR2 | GAS |
| SEQ ID NO: 50 (IMGT) | LCDR3 | QQHNAFPYT |
| SEQ ID NO: 48 | LCDR1 | RASQSVGSNLA |
| (Alternative) | | |
| SEQ ID NO: 49 | LCDR2 | GASTRAT |
| (Alternative) | | |
| SEQ ID NO: 50 | LCDR3 | QQHNAFPYT |
| (Alternative) | | |
| SEQ ID NO: 55 | VL | EIVLTQSPATLSVSPGERATLSCRASQSVGSNLAWYQQK |
| | | PGQAPRLLIYGASTRATGIPARFSGSGSGTEFTLTISSLQS |
| | | EDFAVYYCQQHNAFPYTFGGGTKVEIK |
| SEQ ID NO: 144 | DNA VL | GAAATAGTGTTGACGCAGTCTCCAGCCACCCTGTCTG |
| | | TGTCTCCAGGGGAAAGAGCCACCCTCTCCTGCAGGGC |
| | | CAGTCAGAGTGTTGGCAGCAACTTAGCCTGGTACCAG |
| | | CAGAAACCTGGCCAGGCTCCCAGGCTCCTCATCTATG |
| | | GTGCATCCACCAGGGCCACTGGTATCCCAGCCAGGTT |
| | | CAGTGGCAGTGGGTCTGGGACAGAGTTCACTCTCACC |
| | | ATCAGCAGCCTGCAGTCTGAAGATTTTGCAGTTTATT |
| | | ACTGTCAGCAGCACAATGCCTTCCCTTACACTTTTGG |
| | | CGGAGGGACCAAGGTTGAGATCAAA |
| 377 | | |
| SEQ ID NO: 145 | HCDR1 | GFTFKSYAMS |
| (Combined) | | |
| SEQ ID NO: 146 | HCDR2 | AISGSGSYTYYADSVKG |
| (Combined) | | |
| SEQ ID NO: 37 | HCDR3 | GGLYGSGSYLSDFDL |
| (Combined) | | |
| SEQ ID NO: 137 (Kabat) | HCDR1 | SYAMS |
| SEQ ID NO: 146 (Kabat) | HCDR2 | AISGSGSYTYYADSVKG |
| SEQ ID NO: 37 (Kabat) | HCDR3 | GGLYGSGSYLSDFDL |
| SEQ ID NO: 147 (Chothia) | HCDR1 | GFTFKSY |
| SEQ ID NO: 148 (Chothia) | HCDR2 | SGSGSY |
| SEQ ID NO: 37 (Chothia) | HCDR3 | GGLYGSGSYLSDFDL |
| SEQ ID NO: 149 (IMGT) | HCDR1 | GFTFKSYA |
| SEQ ID NO: 150 (IMGT) | HCDR2 | ISGSGSYT |
| SEQ ID NO: 43 (IMGT) | HCDR3 | ARGGLYGSGSYLSDFDL |
| SEQ ID NO: 194 | HCDR1 | FTFKSYAMS |
| (Alternative) | | |
| SEQ ID NO: 146 | HCDR2 | AISGSGSYTYYADSVKG |
| (Alternative) | | |
| SEQ ID NO: 43 | HCDR3 | ARGGLYGSGSYLSDFDL |
| (Alternative) | | |
| SEQ ID NO: 151 | VH | EVQLLESGGGLVQPGGSLRLSCAASGFTFKSYAMSWVR |
| | | QAPGKGLEWVSAISGSGSYTYYADSVKGRFTISRDNSK |
| | | NTLYLQMNSLRAEDTAVYYCARGGLYGSGSYLSDFDL |
| | | WGRGTLVTVSS |
| SEQ ID NO: 152 | DNA VH | GAGGTGCAGCTGTTGGAGTCTGGGGGAGGCTTGGTAC |
| | | AGCCTGGGGGGTCCCTGAGACTCTCCTGTGCAGCCTC |
| | | TGGATTCACCTTTAAGAGCTATGCCATGAGTTGGGTC |
| | | CGCCAGGCTCCAGGGAAGGGGCTGGAGTGGGTCTCA |
| | | GCTATTAGTGGAAGTGGTTCGTATACATACTACGCAG |
| | | ACTCCGTGAAGGGCCGGTTCACCATCTCCAGAGACAA |
| | | TTCCAAGAACACGCTGTATCTGCAAATGAACAGCCTG |
| | | AGAGCCGAGGACACGGCGGTGTACTACTGCGCCAGA |
| | | GGTGGATTGTACGGAAGCGGAAGCTACTTGAGTGACT |
| | | TCGACCTATGGGGGAGAGGTACCTTGGTCACCGTCTC |
| | | CTCA |
| SEQ ID NO: 48 | LCDR1 | RASQSVGSNLA |
| (Combined) | | |
| SEQ ID NO: 49 | LCDR2 | GASTRAT |
| (Combined) | | |
| SEQ ID NO: 50 | LCDR3 | QQHNAFPYT |
| (Combined) | | |
| SEQ ID NO: 48 (Kabat) | LCDR1 | RASQSVGSNLA |
| SEQ ID NO: 49 (Kabat) | LCDR2 | GASTRAT |
| SEQ ID NO: 50 (Kabat) | LCDR3 | QQHNAFPYT |
| SEQ ID NO: 51 (Chothia) | LCDR1 | SQSVGSN |
| SEQ ID NO: 52 (Chothia) | LCDR2 | GAS |
| SEQ ID NO: 53 (Chothia) | LCDR3 | HNAFPY |
| SEQ ID NO: 54 (IMGT) | LCDR1 | QSVGSN |
| SEQ ID NO: 52 (IMGT) | LCDR2 | GAS |
| SEQ ID NO: 50 (IMGT) | LCDR3 | QQHNAFPYT |
| SEQ ID NO: 48 | LCDR1 | RASQSVGSNLA |
| (Alternative) | | |
| SEQ ID NO: 49 | LCDR2 | GASTRAT |
| (Alternative) | | |
| SEQ ID NO: 50 | LCDR3 | QQHNAFPYT |
| (Alternative) | | |
| SEQ ID NO: 55 | VL | EIVLTQSPATLSVSPGERATLSCRASQSVGSNLAWYQQK |
| | | PGQAPRLLIYGASTRATGIPARFSGSGSGTEFTLTISSLQS |
| | | EDFAVYYCQQHNAFPYTFGGGTKVEIK |
| SEQ ID NO: 144 | DNA VL | GAAATAGTGTTGACGCAGTCTCCAGCCACCCTGTCTG |
| | | TGTCTCCAGGGGAAAGAGCCACCCTCTCCTGCAGGGC |
| | | CAGTCAGAGTGTTGGCAGCAACTTAGCCTGGTACCAG |
| | | CAGAAACCTGGCCAGGCTCCCAGGCTCCTCATCTATG |
| | | GTGCATCCACCAGGGCCACTGGTATCCCAGCCAGGTT |
| | | CAGTGGCAGTGGGTCTGGGACAGAGTTCACTCTCACC |
| | | ATCAGCAGCCTGCAGTCTGAAGATTTTGCAGTTTATT |
| | | ACTGTCAGCAGCACAATGCCTTCCCTTACACTTTTGG |
| | | CGGAGGGACCAAGGTTGAGATCAAA |
| 930 | | |
| SEQ ID NO: 153 | HCDR1 | GFTFSSYAMS |
| (Combined) | | |
| SEQ ID NO: 154 | HCDR2 | AISGSGGSTYYADSVKG |
| (Combined) | | |
| SEQ ID NO: 37 | HCDR3 | GGLYGSGSYLSDFDL |
| (Combined) | | |
| SEQ ID NO: 137 (Kabat) | HCDR1 | SYAMS |
| SEQ ID NO: 154 (Kabat) | HCDR2 | AISGSGGSTYYADSVKG |
| SEQ ID NO: 37 (Kabat) | HCDR3 | GGLYGSGSYLSDFDL |
| SEQ ID NO: 155 (Chothia) | HCDR1 | GFTFSSY |
| SEQ ID NO: 156 (Chothia) | HCDR2 | SGSGGS |
| SEQ ID NO: 37 (Chothia) | HCDR3 | GGLYGSGSYLSDFDL |
| SEQ ID NO: 157 (IMGT) | HCDR1 | GFTFSSYA |
| SEQ ID NO: 158 (IMGT) | HCDR2 | ISGSGGST |
| SEQ ID NO: 43 (IMGT) | HCDR3 | ARGGLYGSGSYLSDFDL |
| SEQ ID NO: 195 | HCDR1 | FTFSSYAMS |
| (Alternative) | | |
| SEQ ID NO: 154 | HCDR2 | AISGSGGSTYYADSVKG |
| (Alternative) | | |
| SEQ ID NO: 43 | HCDR3 | ARGGLYGSGSYLSDFDL |
| (Alternative) | | |
| SEQ ID NO: 159 | VH | EVQLLESGGGLVQPGGSLRLSCAASGFTFSSYAMSWVR |
| | | QAPGKGLEWVSAISGSGGSTYYADSVKGRFTISRDNSK |
| | | NTLYLQMNSLRAEDTAVYYCARGGLYGSGSYLSDFDL |
| | | WGRGTLVTVSS |
| SEQ ID NO: 160 | DNA VH | GAGGTGCAGCTGTTGGAGTCTGGGGGAGGCTTGGTAC |
| | | AGCCTGGGGGGTCCCTGAGACTCTCCTGTGCAGCCTC |
| | | TGGATTCACCTTTAGCAGCTATGCCATGAGCTGGGTC |
| | | CGCCAGGCTCCAGGGAAGGGGCTGGAGTGGGTCTCA |
| | | GCTATTAGTGGTAGTGGTGGTAGCACATACTACGCAG |
| | | ACTCCGTGAAGGGCCGGTTCACCATCTCCAGAGACAA |
| | | TTCCAAGAACACGCTGTATCTGCAAATGAACAGCCTG |
| | | AGAGCCGAGGACACGGCGGTGTACTACTGCGCCAGA |
| | | GGTGGATTGTACGGAAGCGGAAGCTACTTGAGTGACT |
| | | TCGACCTATGGGGGAGAGGTACCTTGGTCACCGTCTC |
| | | CTCA |
| SEQ ID NO: 48 | LCDR1 | RASQSVGSNLA |
| (Combined) | | |
| SEQ ID NO: 49 | LCDR2 | GASTRAT |
| (Combined) | | |
| SEQ ID NO: 50 | LCDR3 | QQHNAFPYT |
| (Combined) | | |
| SEQ ID NO: 48 (Kabat) | LCDR1 | RASQSVGSNLA |
| SEQ ID NO: 49 (Kabat) | LCDR2 | GASTRAT |
| SEQ ID NO: 50 (Kabat) | LCDR3 | QQHNAFPYT |
| SEQ ID NO: 51 (Chothia) | LCDR1 | SQSVGSN |
| SEQ ID NO: 52 (Chothia) | LCDR2 | GAS |
| SEQ ID NO: 53 (Chothia) | LCDR3 | HNAFPY |
| SEQ ID NO: 54 (IMGT) | LCDR1 | QSVGSN |
| SEQ ID NO: 52 (IMGT) | LCDR2 | GAS |
| SEQ ID NO: 50 (IMGT) | LCDR3 | QQHNAFPYT |
| SEQ ID NO: 48 | LCDR1 | RASQSVGSNLA |
| (Alternative) | | |
| SEQ ID NO: 49 | LCDR2 | GASTRAT |
| (Alternative) | | |
| SEQ ID NO: 50 | LCDR3 | QQHNAFPYT |
| (Alternative) | | |
| SEQ ID NO: 55 | VL | EIVLTQSPATLSVSPGERATLSCRASQSVGSNLAWYQQK |
| | | PGQAPRLLIYGASTRATGIPARFSGSGSGTEFTLTISSLQS |
| | | EDFAVYYCQQHNAFPYTFGGGTKVEIK |
| SEQ ID NO: 144 | DNA VL | GAAATAGTGTTGACGCAGTCTCCAGCCACCCTGTCTG |
| | | TGTCTCCAGGGGAAAGAGCCACCCTCTCCTGCAGGGC |
| | | CAGTCAGAGTGTTGGCAGCAACTTAGCCTGGTACCAG |
| | | CAGAAACCTGGCCAGGCTCCCAGGCTCCTCATCTATG |
| | | GTGCATCCACCAGGGCCACTGGTATCCCAGCCAGGTT |
| | | CAGTGGCAGTGGGTCTGGGACAGAGTTCACTCTCACC |
| | | ATCAGCAGCCTGCAGTCTGAAGATTTTGCAGTTTATT |
| | | ACTGTCAGCAGCACAATGCCTTCCCTTACACTTTTGG |
| | | CGGAGGGACCAAGGTTGAGATCAAA |
| 918 | | |
| SEQ ID NO: 161 | HCDR1 | GGSISSYYWS |
| (Combined) | | |
| SEQ ID NO: 162 | HCDR2 | YIYSSGSTNYNPSLKS |
| (Combined) | | |
| SEQ ID NO: 3 (Combined) | HCDR3 | ESQESPYNNWFDP |
| SEQ ID NO: 163 (Kabat) | HCDR1 | SYYWS |
| SEQ ID NO: 162 (Kabat) | HCDR2 | YIYSSGSTNYNPSLKS |
| SEQ ID NO: 3 (Kabat) | HCDR3 | ESQESPYNNWFDP |
| SEQ ID NO: 164 (Chothia) | HCDR1 | GGSISSY |
| SEQ ID NO: 165 (Chothia) | HCDR2 | YSSGS |
| SEQ ID NO: 3 (Chothia) | HCDR3 | ESQESPYNNWFDP |
| SEQ ID NO: 166 (IMGT) | HCDR1 | GGSISSYY |
| SEQ ID NO: 167 (IMGT) | HCDR2 | IYSSGST |
| SEQ ID NO: 9 (IMGT) | HCDR3 | ARESQESPYNNWFDP |
| SEQ ID NO: 200 | HCDR1 | GSISSYYWS |
| (Alternative) | | |
| SEQ ID NO: 162 | HCDR2 | YIYSSGSTNYNPSLKS |
| (Alternative) | | |
| SEQ ID NO: 9 | HCDR3 | ARESQESPYNNWFDP |
| (Alternative) | | |
| SEQ ID NO: 168 | VH | QVQLQESGPGLVKPSETLSLTCTVSGGSISSYYWSWIRQ |
| | | PPGKGLEWIGYIYSSGSTNYNPSLKSRVTISVDTSKNQFS |
| | | LKLSSVTAADTAVYYCARESQESPYNNWFDPWGQGTL |
| | | VTVSS |
| SEQ ID NO: 169 | DNA VH | CAGGTGCAGCTGCAGGAGTCGGGCCCAGGACTGGTG |
| | | AAGCCTTCGGAGACCCTGTCCCTCACCTGCACTGTCT |
| | | CTGGTGGCTCCATCAGTAGTTACTACTGGAGCTGGAT |
| | | CCGGCAGCCCCCAGGGAAGGGACTGGAGTGGATTGG |
| | | GTATATCTATAGTAGTGGGAGCACCAACTACAACCCC |
| | | TCCCTCAAGAGTCGAGTCACCATATCAGTAGACACGT |
| | | CCAAGAACCAGTTCTCCCTGAAGCTGAGTTCTGTGAC |
| | | CGCCGCAGACACGGCGGTGTACTACTGCGCCAGAGA |
| | | ATCTCAGGAGAGTCCATACAACAATTGGTTCGACCCA |
| | | TGGGGACAGGGTACATTGGTCACCGTCTCCTCA |
| SEQ ID NO: 14 | LCDR1 | RASQGISSWLA |
| (Combined) | | |
| SEQ ID NO: 15 | LCDR2 | AASSLQS |
| (Combined) | | |
| SEQ ID NO: 16 | LCDR3 | QQGNSFPRT |
| (Combined) | | |
| SEQ ID NO: 14 (Kabat) | LCDR1 | RASQGISSWLA |
| SEQ ID NO: 15 (Kabat) | LCDR2 | AASSLQS |
| SEQ ID NO: 16 (Kabat) | LCDR3 | QQGNSFPRT |
| SEQ ID NO: 17 (Chothia) | LCDR1 | SQGISSW |
| SEQ ID NO: 18 (Chothia) | LCDR2 | AAS |
| SEQ ID NO: 19 (Chothia) | LCDR3 | GNSFPR |
| SEQ ID NO: 20 (IMGT) | LCDR1 | QGISSW |
| SEQ ID NO: 18 (IMGT) | LCDR2 | AAS |
| SEQ ID NO: 16 (IMGT) | LCDR3 | QQGNSFPRT |
| SEQ ID NO: 14 | LCDR1 | RASQGISSWLA |
| (Alternative) | | |
| SEQ ID NO: 15 | LCDR2 | AASSLQS |
| (Alternative) | | |
| SEQ ID NO: 16 | LCDR3 | QQGNSFPRT |
| (Alternative) | | |
| SEQ ID NO: 21 | VL | DIQMTQSPSSVSASVGDRVTITCRASQGISSWLAWYQQ |
| | | KPGKAPKLLIYAASSLQSGVPSRFSGSGSGTDFTLTISSL |
| | | QPEDFATYYCQQGNSFPRTFGGGTKVEIK |
| SEQ ID NO: 170 | DNA VL | GACATCCAGATGACCCAGTCTCCATCTTCCGTGTCTG |
| | | CATCTGTAGGAGACAGAGTCACCATCACTTGTCGGGC |
| | | GAGTCAGGGTATTAGCAGCTGGTTAGCCTGGTATCAG |
| | | CAGAAACCAGGGAAAGCCCCTAAGCTCCTGATCTATG |
| | | CTGCATCCAGTTTGCAAAGTGGGGTCCCATCAAGGTT |
| | | CAGCGGCAGTGGATCTGGGACAGATTTCACTCTCACC |
| | | ATCAGCAGCCTGCAGCCTGAAGATTTTGCAACTTATT |
| | | ACTGTCAGCAGGGAAACAGTTTCCCTAGGACTTTTGG |
| | | CGGAGGGACCAAGGTTGAGATCAAA |
| 913 | | |
| SEQ ID NO: 210 | HCDR1 | GGSFSGYYWS |
| (Combined) | | |
| SEQ ID NO: 211 | HCDR2 | EIDHSGSTNYNPSLKS |
| (Combined) | | |
| SEQ ID NO: 212 | HCDR3 | EGGLGEDYYYYGMDV |
| (Combined) | | |
| SEQ ID NO: 213 (Kabat) | HCDR1 | GYYWS |
| SEQ ID NO: 211 (Kabat) | HCDR2 | EIDHSGSTNYNPSLKS |
| SEQ ID NO: 212 (Kabat) | HCDR3 | EGGLGEDYYYYGMDV |
| SEQ ID NO: 214 (Chothia) | HCDR1 | GGSFSGY |
| SEQ ID NO: 215 (Chothia) | HCDR2 | DHSGS |
| SEQ ID NO: 212 (Chothia) | HCDR3 | EGGLGEDYYYYGMDV |
| SEQ ID NO: 216 (IMGT) | HCDR1 | GGSFSGYY |
| SEQ ID NO: 217 (IMGT) | HCDR2 | IDHSGST |
| SEQ ID NO: 218 (IMGT) | HCDR3 | AREGGLGEDYYYYGMDV |
| SEQ ID NO: 219 | HCDR1 | GSFSGYYWS |
| (Alternative) | | |
| SEQ ID NO: 211 | HCDR2 | EIDHSGSTNYNPSLKS |
| (Alternative) | | |
| SEQ ID NO: 218 | HCDR3 | AREGGLGEDYYYYGMDV |
| (Alternative) | | |
| SEQ ID NO: 220 | VH | QVQLQQWGAGLLKPSETLSLTCAVYGGSFSGYYWSWI |
| | | RQPPGKGLEWIGEIDHSGSTNYNPSLKSRVTISVDTSKN |
| | | QFSLKLSSVTAADTAVYYCAREGGLGEDYYYYGMDV |
| | | WGQGTTVTVSS |
| SEQ ID NO: 221 | LCDR1 | RASQSISSYLN |
| (Combined) | | |
| SEQ ID NO: 222 | LCDR2 | GASSLQS |
| (Combined) | | |
| SEQ ID NO: 223 | LCDR3 | QQQYPLPPT |
| (Combined) | | |
| SEQ ID NO: 221 (Kabat) | LCDR1 | RASQSISSYLN |
| SEQ ID NO: 222 (Kabat) | LCDR2 | GASSLQS |
| SEQ ID NO: 223 (Kabat) | LCDR3 | QQQYPLPPT |
| SEQ ID NO: 224 (Chothia) | LCDR1 | SQSISSY |
| SEQ ID NO: 52 (Chothia) | LCDR2 | GAS |
| SEQ ID NO: 225 (Chothia) | LCDR3 | QYPLPP |
| SEQ ID NO: 226 (IMGT) | LCDR1 | QSISSY |
| SEQ ID NO: 52 (IMGT) | LCDR2 | GAS |
| SEQ ID NO: 223 (IMGT) | LCDR3 | QQQYPLPPT |
| SEQ ID NO: 221 | LCDR1 | RASQSISSYLN |
| (Alternative) | | |
| SEQ ID NO: 222 | LCDR2 | GASSLQS |
| (Alternative) | | |
| SEQ ID NO: 223 | LCDR3 | QQQYPLPPT |
| (Alternative) | | |
| SEQ ID NO: 227 | VL | DIQMTQSPSSLSASVGDRVTITCRASQSISSYLNWYQQK |
| | | PGKAPKLLIYGASSLQSGVPSRFSGSGSGTDFTLTISSLQP |
| | | EDFATYYCQQQYPLPPTFGGGTKVEIK |
| 908 | | |
| SEQ ID NO: 333 | HCDR1 | GGTFSSLAIS |
| (Combined) | | |
| SEQ ID NO: 288 | HCDR2 | GIIPIFGTANYAQKFQG |
| (Combined) | | |
| SEQ ID NO: 334 | HCDR3 | EGAGWYYAFDI |
| (Combined) | | |
| SEQ ID NO: 335 (Kabat) | HCDR1 | SLAIS |
| SEQ ID NO: 288 (Kabat) | HCDR2 | GIIPIFGTANYAQKFQG |
| SEQ ID NO: 334 (Kabat) | HCDR3 | EGAGWYYAFDI |
| SEQ ID NO: 336 (Chothia) | HCDR1 | GGTFSSL |
| SEQ ID NO: 292 (Chothia) | HCDR2 | IPIFGT |
| SEQ ID NO: 334 (Chothia) | HCDR3 | EGAGWYYAFDI |
| SEQ ID NO: 337 (IMGT) | HCDR1 | GGTFSSLA |
| SEQ ID NO: 294 (IMGT) | HCDR2 | IIPIFGTA |
| SEQ ID NO: 338 (IMGT) | HCDR3 | AREGAGWYYAFDI |
| SEQ ID NO: 339 | HCDR1 | GTFSSLAIS |
| (Alternative) | | |
| SEQ ID NO: 288 | HCDR2 | GIIPIFGTANYAQKFQG |
| (Alternative) | | |
| SEQ ID NO: 338 | HCDR3 | AREGAGWYYAFDI |
| (Alternative) | | |
| SEQ ID NO: 340 | VH | QVQLVQSGAEVKKPGSSVKVSCKASGGTFSSLAISWVR |
| | | QAPGQGLEWMGGIIPIFGTANYAQKFQGRVTITADESTS |
| | | TAYMELSSLRSEDTAVYYCAREGAGWYYAFDIWGQGT |
| | | MVTVSS |
| SEQ ID NO: 221 | LCDR1 | RASQSISSYLN |
| (Combined) | | |
| SEQ ID NO: 222 | LCDR2 | GASSLQS |
| (Combined) | | |
| SEQ ID NO: 341 | LCDR3 | QQAYVKPIT |
| (Combined) | | |
| SEQ ID NO: 221 (Kabat) | LCDR1 | RASQSISSYLN |
| SEQ ID NO: 222 (Kabat) | LCDR2 | GASSLQS |
| SEQ ID NO: 341 (Kabat) | LCDR3 | QQAYVKPIT |
| SEQ ID NO: 224 (Chothia) | LCDR1 | SQSISSY |
| SEQ ID NO: 52 (Chothia) | LCDR2 | GAS |
| SEQ ID NO: 342 (Chothia) | LCDR3 | AYVKPI |
| SEQ ID NO: 226 (IMGT) | LCDR1 | QSISSY |
| SEQ ID NO: 52 (IMGT) | LCDR2 | GAS |
| SEQ ID NO: 341 (IMGT) | LCDR3 | QQAYVKPIT |
| SEQ ID NO: 221 | LCDR1 | RASQSISSYLN |
| (Alternative) | | |
| SEQ ID NO: 222 | LCDR2 | GASSLQS |
| (Alternative) | | |
| SEQ ID NO: 341 | LCDR3 | QQAYVKPIT |
| (Alternative) | | |
| SEQ ID NO: 343 | VL | DIQMTQSPSSLSASVGDRVTITCRASQSISSYLNWYQQK |
| | | PGKAPKLLIYGASSLQSGVPSRFSGSGSGTDFTLTISSLQP |
| | | EDFATYYCQQAYVKPITFGGGTKVEIK |
| 421 | | |
| SEQ ID NO: 270 | HCDR1 | GGSILSSSYYWV |
| (Combined) | | |
| SEQ ID NO: 271 | HCDR2 | SIYYSGGTDYNPSLKS |
| (Combined) | | |
| SEQ ID NO: 272 | HCDR3 | VMSGWLDLDV |
| (Combined) | | |
| SEQ ID NO: 273 (Kabat) | HCDR1 | SSSYYWV |
| SEQ ID NO: 271 (Kabat) | HCDR2 | SIYYSGGTDYNPSLKS |
| SEQ ID NO: 272 (Kabat) | HCDR3 | VMSGWLDLDV |
| SEQ ID NO: 274 (Chothia) | HCDR1 | GGSILSSSY |
| SEQ ID NO: 275 (Chothia) | HCDR2 | YYSGG |
| SEQ ID NO: 272 (Chothia) | HCDR3 | VMSGWLDLDV |
| SEQ ID NO: 276 (IMGT) | HCDR1 | GGSILSSSYY |
| SEQ ID NO: 277 (IMGT) | HCDR2 | IYYSGGT |
| SEQ ID NO: 278 (IMGT) | HCDR3 | ARVMSGWLDLDV |
| SEQ ID NO: 279 | HCDR1 | GSILSSSYYWV |
| (Alternative) | | |
| SEQ ID NO: 271 | HCDR2 | SIYYSGGTDYNPSLKS |
| (Alternative) | | |
| SEQ ID NO: 278 | HCDR3 | ARVMSGWLDLDV |
| (Alternative) | | |
| SEQ ID NO: 280 | VH | QLQLQESGPGLVKPSETLSLTCTVSGGSILSSSYYWVWI |
| | | RQPPGKGLEWIGSIYYSGGTDYNPSLKSRVTISVDTSKN |
| | | QFSLKLSSVTAADTAVYYCARVMSGWLDLDVWGQGT |
| | | MVTVSS |
| SEQ ID NO: 281 | LCDR1 | RASQSINSYLN |
| (Combined) | | |
| SEQ ID NO: 15 | LCDR2 | AASSLQS |
| (Combined) | | |
| SEQ ID NO: 282 | LCDR3 | QQAYARPRT |
| (Combined) | | |
| SEQ ID NO: 281 (Kabat) | LCDR1 | RASQSINSYLN |
| SEQ ID NO: 15 (Kabat) | LCDR2 | AASSLQS |
| SEQ ID NO: 282 (Kabat) | LCDR3 | QQAYARPRT |
| SEQ ID NO: 283 (Chothia) | LCDR1 | SQSINSY |
| SEQ ID NO: 18 (Chothia) | LCDR2 | AAS |
| SEQ ID NO: 284 (Chothia) | LCDR3 | AYARPR |
| SEQ ID NO: 285 (IMGT) | LCDR1 | QSINSY |
| SEQ ID NO: 18 (IMGT) | LCDR2 | AAS |
| SEQ ID NO: 282 (IMGT) | LCDR3 | QQAYARPRT |
| SEQ ID NO: 281 | LCDR1 | RASQSINSYLN |
| (Alternative) | | |
| SEQ ID NO: 15 | LCDR2 | AASSLQS |
| (Alternative) | | |
| SEQ ID NO: 282 | LCDR3 | QQAYARPRT |
| (Alternative) | | |
| SEQ ID NO: 286 | VL | DIQMTQSPSSLSASVGDRVTITCRASQSINSYLNWYQQK |
| | | PGKAPKLLIYAASSLQSGVPSRFSGSGSGTDFTLTISSLQP |
| | | EDFATYYCQQAYARPRTFGGGTKVEIK |
| 939 | | |
| SEQ ID NO: 287 | HCDR1 | GGTFSSYAIS |
| (Combined) | | |
| SEQ ID NO: 288 | HCDR2 | GIIPIFGTANYAQKFQG |
| (Combined) | | |
| SEQ ID NO: 289 | HCDR3 | HYYDYWSGYYTNTGIY |
| (Combined) | | |
| SEQ ID NO: 290 (Kabat) | HCDR1 | SYAIS |
| SEQ ID NO: 288 (Kabat) | HCDR2 | GIIPIFGTANYAQKFQG |
| SEQ ID NO: 289 (Kabat) | HCDR3 | HYYDYWSGYYTNTGIY |
| SEQ ID NO: 291 (Chothia) | HCDR1 | GGTFSSY |
| SEQ ID NO: 292 (Chothia) | HCDR2 | IPIFGT |
| SEQ ID NO: 289 (Chothia) | HCDR3 | HYYDYWSGYYTNTGIY |
| SEQ ID NO: 293 (IMGT) | HCDR1 | GGTFSSYA |
| SEQ ID NO: 294 (IMGT) | HCDR2 | IIPIFGTA |
| SEQ ID NO: 295 (IMGT) | HCDR3 | ARHYYDYWSGYYTNTGIY |
| SEQ ID NO: 296 | HCDR1 | GTFSSYAIS |
| (Alternative) | | |
| SEQ ID NO: 288 | HCDR2 | GIIPIFGTANYAQKFQG |
| (Alternative) | | |
| SEQ ID NO: 295 | HCDR3 | ARHYYDYWSGYYTNTGIY |
| (Alternative) | | |
| SEQ ID NO: 297 | VH | QVQLVQSGAEVKKPGSSVKVSCKASGGTFSSYAISWVR |
| | | QAPGQGLEWMGGIIPIFGTANYAQKFQGRVTITADESTS |
| | | TAYMELSSLRSEDTAVYYCARHYYDYWSGYYTNTGIY |
| | | WGQGTLVTVSS |
| SEQ ID NO: 298 | LCDR1 | RASQSVSSNLA |
| (Combined) | | |
| SEQ ID NO: 49 | LCDR2 | GASTRAT |
| (Combined) | | |
| SEQ ID NO: 299 | LCDR3 | QQSGALPIT |
| (Combined) | | |
| SEQ ID NO: 298 (Kabat) | LCDR1 | RASQSVSSNLA |
| SEQ ID NO: 49 (Kabat) | LCDR2 | GASTRAT |
| SEQ ID NO: 299 (Kabat) | LCDR3 | QQSGALPIT |
| SEQ ID NO: 300 (Chothia) | LCDR1 | SQSVSSN |
| SEQ ID NO: 52 (Chothia) | LCDR2 | GAS |
| SEQ ID NO: 301 (Chothia) | LCDR3 | SGALPI |
| SEQ ID NO: 302 (IMGT) | LCDR1 | QSVSSN |
| SEQ ID NO: 52 (IMGT) | LCDR2 | GAS |
| SEQ ID NO: 299 (IMGT) | LCDR3 | QQSGALPIT |
| SEQ ID NO: 298 | LCDR1 | RASQSVSSNLA |
| (Alternative) | | |
| SEQ ID NO: 49 | LCDR2 | GASTRAT |
| (Alternative) | | |
| SEQ ID NO: 299 | LCDR3 | QQSGALPIT |
| (Alternative) | | |
| SEQ ID NO: 303 | VL | EIVMTQSPATLSVSPGERATLSCRASQSVSSNLAWYQQK |
| | | PGQAPRLLIYGASTRATGIPARFSGSGSGTEFTLTISSLQS |
| | | EDFAVYYCQQSGALPITFGGGTKVEIK |

TABLE 26
Consensus CDR sequences for exemplary anti-CD73 antibodies

| Kabat | HCDR1 | HCDR2 | HCDR3 | LCDR1 | LCDR2 | LCDR3 |
|---|---|---|---|---|---|---|
| 913 | GYYWS | EIDHSGST | EGGLGED | RASQSISS | GASSLQS | QQQYPLPP |
| | (SEQ ID | NYNPSLKS | YYYYGMD | YLN SEQ | (SEQ ID | T (SEQ ID |
| | NO: 213) | (SEQ ID | V (SEQ ID | ID NO: 221) | NO: 222) | NO: 223) |
| | | NO: 211) | NO: 212) | | | |
| 908 | SLAIS (SEQ | GIIPIFGTA | EGAGWYY | RASQSISS | GASSLQS | QQAYVKPI |
| | ID NO: 335) | NYAQKFQ | AFDI (SEQ | YLN (SEQ | (SEQ ID | T (SEQ ID |
| | | G (SEQ ID | ID NO: 334) | ID NO: 221) | NO: 222) | NO: 341) |
| | | NO: 288) | | | | |
| 421 | SSSYYWV | SIYYSGGT | VMSGWLD | RASQSINS | AASSLQS | QQAYARP |
| | (SEQ ID | DYNPSLKS | LDV (SEQ | YLN (SEQ | (SEQ ID | RT (SEQ ID |
| | NO: 273) | (SEQ ID | ID NO: 272) | ID NO: 281) | NO: 15) | NO: 282) |
| | | NO: 271) | | | | |
| Consensus | | | | RASQSIXS | XASSLQS, | QQX₁YX₂X₃ |
| | | | | YLN, | wherein X is | PX₄T, |
| | | | | wherein X is | G or A (SEQ | wherein: |
| | | | | S or N (SEQ | ID NO: 345) | X₁ is Q or A; |
| | | | | ID NO: 344) | | X₂ is P, V, |
| | | | | | | or A; |
| | | | | | | X₃ is L, K, |
| | | | | | | or R; and |
| | | | | | | X₄ is P, I, or |
| | | | | | | R (SEQ ID |
| | | | | | | NO: 346) |

TABLE 27
Consensus CDR sequences for exemplary anti-CD73 antibodies

| Kabat | HCDR1 | HCDR2 | HCDR3 | LCDR1 | LCDR2 | LCDR3 |
|---|---|---|---|---|---|---|
| 908 | SLAIS (SEQ | GIIPIFGTA | EGAGWYY | RASQSISS | GASSLQS | QQAYVKPI |
| | ID NO: 335) | NYAQKFQ | AFDI (SEQ | YLN (SEQ | (SEQ ID | T (SEQ ID |
| | | G (SEQ ID | ID NO: 334) | ID NO: 221) | NO: 222) | NO: 341) |
| | | NO: 288) | | | | |
| 939 | SYAIS | GIIPIFGTA | HYYDYWS | RASQSVSS | GASTRAT | QQSGALPI |
| | (SEQ ID | NYAQKFQ | GYYTNTGI | NLA (SEQ | (SEQ ID | T (SEQ ID |
| | NO: 290) | G (SEQ ID | Y (SEQ ID | ID NO: 298) | NO: 49) | NO: 299) |
| | | NO: 288) | NO: 289) | | | |
| Consensus | SXAIS, | GIIPIFGTA | | | | |
| | wherein X is | NYAQKFQ | | | | |
| | L or Y (SEQ | G (SEQ ID | | | | |
| | ID NO: 347) | NO: 288) | | | | |

TABLE 28
Corresponding germline sequences
of exemplary anti-CD73 antibodies

| Antibody | VH germline | VL germline |
|---|---|---|
| 913 | VH4-34 | VK1-39 |
| 908 | VH1-69 | VK1-39 |
| 421 | VH4-39 | VK1-39 |
| 939 | VH1-69 | VK3-15 |

TABLE 2
Consensus CDR sequences for exemplary anti-CD73 antibodies

| | | |
|---|---|---|
| Lineage 3 | | |
| SEQ ID NO: 126 | HCDR1 | GFTFX₁X₂X₃AMS, wherein |
| (Combined) | | X₁ is H, S, R, or K; |
| | | X₂ is R, Y, or S; and |
| | | X₃ is Y or N |
| SEQ ID NO: 89 | HCDR2 | X₁IX₂GX₃GX₄X₅TYYADSVKG, wherein |
| (Combined) | | X₁ is A or S; |
| | | X₂ is S or T; |
| | | X₃ is S or T; |
| | | X₄ is M, G, or S; and |
| | | X₅ is N, S, L, or Y |
| SEQ ID NO: 37 | HCDR3 | GGLYGSGSYLSDFDL |
| (Combined) | | |
| SEQ ID NO: 48 | LCDR1 | RASQSVGSNLA |
| (Combined) | | |
| SEQ ID NO: 49 | LCDR2 | GASTRAT |
| (Combined) | | |
| SEQ ID NO: 50 | LCDR3 | QQHNAFPYT |
| (Combined) | | |
| SEQ ID NO: 88 (Kabat) | HCDR1 | X₁X₂AMS, wherein |
| | | X₁ is R, Y, or S; and |
| | | X₂ is Y or N |
| SEQ ID NO: 89 (Kabat) | HCDR2 | X₁IX₂GX₃GX₄X₅TYYADSVKG, wherein |
| | | X₁ is A or S; |
| | | X₂ is S or T; |
| | | X₃ is S or T; |
| | | X₄ is M, G, or S; and |
| | | X₅ is N, S, L, or Y |
| SEQ ID NO: 37 (Kabat) | HCDR3 | GGLYGSGSYLSDFDL |
| SEQ ID NO: 48 (Kabat) | LCDR1 | RASQSVGSNLA |
| SEQ ID NO: 49 (Kabat) | LCDR2 | GASTRAT |
| SEQ ID NO: 50 (Kabat) | LCDR3 | QQHNAFPYT |
| SEQ ID NO: 122 (Chothia) | HCDR1 | GFTFX₁X₂X₃, wherein |
| | | X₁ is H, S, R, or K; |
| | | X₂ is R, Y, or S; and |
| | | X₃ is Y or N |
| SEQ ID NO: 123 (Chothia) | HCDR2 | X₁GX₂GX₃X₄, wherein |
| | | X₁ is S or T; |
| | | X₂ is S or T; |
| | | X₃ is M, G, or S; and |
| | | X₄ is N, S, L, or Y |
| SEQ ID NO: 37 (Chothia) | HCDR3 | GGLYGSGSYLSDFDL |
| SEQ ID NO: 51 (Chothia) | LCDR1 | SQSVGSN |
| SEQ ID NO: 52 (Chothia) | LCDR2 | GAS |
| SEQ ID NO: 53 (Chothia) | LCDR3 | HNAFPY |
| SEQ ID NO: 128 (IMGT) | HCDR1 | GFTFX₁X₂X₃A, wherein |
| | | X₁ is H, S, R, or K; |
| | | X₂ is R, Y, or S; and |
| | | X₃ is Y or N |
| SEQ ID NO: 129 (IMGT) | HCDR2 | IX₁GX₂GX₃X₄T, wherein |
| | | X₁ is S or T; |
| | | X₂ is S or T; |
| | | X₃ is M, G, or S; and |
| | | X₄ is N, S, L, or Y |
| SEQ ID NO: 43 (IMGT) | HCDR3 | ARGGLYGSGSYLSDFDL |
| SEQ ID NO: 54 (IMGT) | LCDR1 | QSVGSN |
| SEQ ID NO: 52 (IMGT) | LCDR2 | GAS |
| SEQ ID NO: 50 (IMGT) | LCDR3 | QQHNAFPYT |
| SEQ ID NO: 189 | HCDR1 | FTFX₁X₂X₃AMS, wherein |
| (Alternative) | | X₁ is H, S, R, or K; |
| | | X₂ is R, Y, or S; and |
| | | X₃ is Y or N |
| SEQ ID NO: 89 | HCDR2 | X₁IX₂GX₃GX₄X₅TYYADSVKG, wherein |
| (Alternative) | | X₁ is A or S; |
| | | X₂ is S or T; |
| | | X₃ is S or T; |
| | | X₄ is M, G, or S; and |
| | | X₅ is N, S, L, or Y |
| SEQ ID NO: 43 | HCDR3 | ARGGLYGSGSYLSDFDL |
| (Alternative) | | |
| SEQ ID NO: 48 | LCDR1 | RASQSVGSNLA |
| (Alternative) | | |
| SEQ ID NO: 49 | LCDR2 | GASTRAT |
| (Alternative) | | |
| SEQ ID NO: 50 | LCDR3 | QQHNAFPYT |
| (Alternative) | | |
| Lineage 1 | | |
| SEQ ID NO: 127 | HCDR1 | GGSIX₁X₂X₃YWS, wherein |
| (Combined) | | X₁ is E or S; |
| | | X₂ is R, G, or S; and |
| | | X₃ is Y or R |
| SEQ ID NO: 91 | HCDR2 | YIYX₁X₂GSTX₃YNPSLKS, wherein |
| (Combined) | | X₁ is G or S; |
| | | X₂ is R, S, or T; and |
| | | X₃ is N or K |
| SEQ ID NO: 3 (Combined) | HCDR3 | ESQESPYNNWFDP |
| SEQ ID NO: 14 | LCDR1 | RASQGISSWLA |
| (Combined) | | |
| SEQ ID NO: 15 | LCDR2 | AASSLQS |
| (Combined) | | |
| SEQ ID NO: 16 | LCDR3 | QQGNSFPRT |
| (Combined) | | |
| SEQ ID NO: 90 (Kabat) | HCDR1 | X₁X₂YWS, wherein |
| | | X₁ is R, G, or S; and |
| | | X₂ is Y or R |
| SEQ ID NO: 91 (Kabat) | HCDR2 | YIYX₁X₂GSTX₃YNPSLKS, wherein |
| | | X₁ is G or S; |
| | | X₂ is R, S, or T; and |
| | | X₃ is N or K |
| SEQ ID NO: 3 (Kabat) | HCDR3 | ESQESPYNNWFDP |
| SEQ ID NO: 14 (Kabat) | LCDR1 | RASQGISSWLA |
| SEQ ID NO: 15 (Kabat) | LCDR2 | AASSLQS |
| SEQ ID NO: 16 (Kabat) | LCDR3 | QQGNSFPRT |
| SEQ ID NO: 124 (Chothia) | HCDR1 | GGSIX₁X₂X₃, wherein |
| | | X₁ is E or S; |
| | | X₂ is R, G, or S; and |
| | | X₃ is Y or R |
| SEQ ID NO: 125 (Chothia) | HCDR2 | YX₁X₂GS, wherein |
| | | X₁ is G or S; and |
| | | X₂ is R, S, or T |
| SEQ ID NO: 3 (Chothia) | HCDR3 | ESQESPYNNWFDP |
| SEQ ID NO: 17 (Chothia) | LCDR1 | SQGISSW |
| SEQ ID NO: 18 (Chothia) | LCDR2 | AAS |
| SEQ ID NO: 19 (Chothia) | LCDR3 | GNSFPR |
| SEQ ID NO: 130 (IMGT) | HCDR1 | GGSIX₁X₂X₃Y, wherein |
| | | X₁ is E or S; |
| | | X₂ is R, G, or S; and |
| | | X₃ is Y or R |
| SEQ ID NO: 131 (IMGT) | HCDR2 | IYX₁X₂GST, wherein |
| | | X₁ is G or S; and |
| | | X₂ is R, S, or T |
| SEQ ID NO: 9 (IMGT) | HCDR3 | ARESQESPYNNWFDP |
| SEQ ID NO: 20 (IMGT) | LCDR1 | QGISSW |
| SEQ ID NO: 18 (IMGT) | LCDR2 | AAS |
| SEQ ID NO: 16 (IMGT) | LCDR3 | QQGNSFPRT |
| SEQ ID NO: 196 | HCDR1 | GSIX₁X₂X₃YWS, wherein |
| (Alternative) | | X₁ is E or S; |
| | | X₂ is R, G, or S; and |
| | | X₃ is Y or R |
| SEQ ID NO: 91 | HCDR2 | YIYX₁X₂GSTX₃YNPSLKS, wherein |
| (Alternative) | | X₁ is G or S; |
| | | X₂ is R, S, or T; and |
| | | X₃ is N or K |
| SEQ ID NO: 9 | HCDR3 | ARESQESPYNNWFDP |
| (Alternative) | | |
| SEQ ID NO: 14 | LCDR1 | RASQGISSWLA |
| (Alternative) | | |
| SEQ ID NO: 15 | LCDR2 | AASSLQS |
| (Alternative) | | |
| SEQ ID NO: 16 | LCDR3 | QQGNSFPRT |
| (Alternative) | | |

TABLE 3
Constant region amino acid sequences of
human IgG heavy chains and human kappa
light chain

| SEQ | | |
|---|---|---|
| ID | | |
| NO | Description | Sequence |
| 92 | IgG4 | ASTKGPSVFPLAPCSRSTSESTAALGC |
| | (S228P) | LVKDYFPEPVTVSWNSGALTSGVHTFP |
| | | AVLQSSGLYSLSSVVTVPSSSLGTKTY |
| | | TCNVDHKPSNTKVDKRVESKYGPPCPP |
| | | CPAPEFLGGPSVFLFPPKPKDTLMISR |
| | | TPEVTCVVVDVSQEDPEVQFNWYVDGV |
| | | EVHNAKTKPREEQFNSTYRVVSVLTVL |
| | | HQDWLNGKEYKCKVSNKGLPSSIEKTI |
| | | SKAKGQPREPQVYTLPPSQEEMTKNQV |
| | | SLTCLVKGFYPSDIAVEWESNGQPENN |
| | | YKTTPPVLDSDGSFFLYSRLTVDKSRW |
| | | QEGNVFSCSVMHEALHNHYTQKSLSLS |
| | | LGK |
| 93 | IgG4 | ASTKGPSVFPLAPCSRSTSESTAALGC |
| | (S228P) | LVKDYFPEPVTVSWNSGALTSGVHTFP |
| | lacking C- | AVLQSSGLYSLSSVVTVPSSSLGTKTY |
| | terminal | TCNVDHKPSNTKVDKRVESKYGPPCPP |
| | lysine | CPAPEFLGGPSVFLFPPKPKDTLMISR |
| | | TPEVTCVVVDVSQEDPEVQFNWYVDGV |
| | | EVHNAKTKPREEQFNSTYRVVSVLTVL |
| | | HQDWLNGKEYKCKVSNKGLPSSIEKTI |
| | | SKAKGQPREPQVYTLPPSQEEMTKNQV |
| | | SLTCLVKGFYPSDIAVEWESNGQPENN |
| | | YKTTPPVLDSDGSFFLYSRLTVDKSRW |
| | | QEGNVFSCSVMHEALHNHYTQKSLSLS |
| | | LG |
| 94 | IgG4 | ASTKGPSVFPLAPCSRSTSESTAALGC |
| | (S228P/ | LVKDYFPEPVTVSWNSGALTSGVHTFP |
| | L235E) | AVLQSSGLYSLSSVVTVPSSSLGTKTY |
| | | TCNVDHKPSNTKVDKRVESKYGPPCPP |
| | | CPAPEFEGGPSVFLFPPKPKDTLMISR |
| | | TPEVTCVVVDVSQEDPEVQFNWYVDGV |
| | | EVHNAKTKPREEQFNSTYRVVSVLTVL |
| | | HQDWLNGKEYKCKVSNKGLPSSIEKTI |
| | | SKAKGQPREPQVYTLPPSQEEMTKNQV |
| | | SLTCLVKGFYPSD1AVEWESNGQPENN |
| | | YKTTPPVLDSDGSFFLYSRLTVDKSRW |
| | | QEGNVFSCSVMHEALHNHYTQKSLSLS |
| | | LGK |
| 95 | IgG4 | ASTKGPSVFPLAPCSRSTSESTAALGC |
| | (S228P/ | LVKDYFPEPVTVSWNSGALTSGVHTFP |
| | L235E) | AVLQSSGLYSLSSVVTVPSSSLGTKTY |
| | lacking C- | TCNVDHKPSNTKVDKRVESKYGPPCPP |
| | tenninal | CPAPEFEGGPSVFLFPPKPKDTLMISR |
| | lysine | TPEVTCVVVDVSQEDPEVQFNWYVDGV |
| | | EVHNAKTKPREEQFNSTYRVVSVLTVL |
| | | HQDWLNGKEYKCKVSNKGLPSSIEKTI |
| | | SKAKGQPREPQVYTLPPSQEE |
| | | MTKNQVSLTCLVKGFYPSDIAVEWESN |
| | | GQPENNYKTTPPVLDSDGSFFLYSRLT |
| | | VDKSRWQEGNVFSCSVMHEALHNHYTQ |
| | | KSLSLSLG |
| 119 | IgG1 | ASTKGPSVFPLAPSSKSTSGGTAALGC |
| | wild type | LVKDYFPEPVTVSWNSGALTSGVHTFP |
| | | AVLQSSGLYSLSSVVTVPSSSLGTQTY |
| | | ICNVNHKPSNTKVDKKVEPKSCDKTHT |
| | | CPPCPAPELLGGPSVFLFPPKPKDTLM |
| | | ISRTPEVTCVVVDVSHEDPEVKFNWYV |
| | | DGVEVHNAKTKPREEQYNSTYRVVSVL |
| | | TVLHQDWLNGKEYKCKVSNKALPAPIE |
| | | KTISKAKGQPREPQVYTLPPSRDELTK |
| | | NQVSLTCLVKGFYPSDIAVEWESNGQP |
| | | ENNYKTTPPVLDSDGSFFLYSKLTVDK |
| | | SRWQQGNVFSCSVMHEALHNHYTQKSL |
| | | SLSPGK |
| 120 | IgG1 | ASTKGPSVFPLAPSSKSTSGGTAALGC |
| | wild type | LVKDYFPEPVTVSWNSGALTSGVHTFP |
| | lacking C- | AVLQSSGLYSLSSVVTVPSSSLGTQTY |
| | terininal | ICNVNHKPSNTKVDKKVEPKSCDKTHT |
| | lysine | CPPCPAPELLGGPSVFLFPPKPKDTLM |
| | | ISRTPEVTCVVVDVSHEDPEVKFNWYV |
| | | DGVEVHNAKTKPREEQYNSTYRVVSVL |
| | | TVLHQDWLNGKEYKCKVSNKALPAPIE |
| | | KTISKAKGQPREPQVYTLPPSRDELTK |
| | | NQVSLTCLVKGFYPSDIAVEWESNGQP |
| | | ENNYKTTPPVLDSDGSFFLYSKLTVDK |
| | | SRWQQGNVFSCSVMHEALHNHYTQKSL |
| | | SLSPG |
| 96 | IgG1 | ASTKGPSVFPLAPSSKSTSGGTAALGC |
| | wild type | LVKDYFPEPVTVSWNSGALTSGVHTFP |
| | | AVLQSSGLYSLSSVVTVPSSSLGTQTY |
| | | ICNVNHKPSNTKVDKRVEPKSCDKTHT |
| | | CPPCPAPELLGGPSVFLFPPKPKDTLM |
| | | ISRTPEVTCVVVDVSHEDPEVKFNWYV |
| | | DGVEVHNAKTKPREEQYNSTYRVVSVL |
| | | TVLHQDWLNGKEYKCKVSNKALPAPIE |
| | | KTISKAKGQPREPQVYTLPPSREEMTK |
| | | NQVSLTCLVKGFYPSDIAVEWESNGQP |
| | | ENNYKTTPPVLDSDGSFFLYSKLTVDK |
| | | SRWQQGNVFSCSVMHEALHNHYTQKSL |
| | | SLSPGK |
| 97 | IgG1 | ASTKGPSVFPLAPSSKSTSGGTAALGC |
| | wild type | LVKDYFPEPVTVSWNSGALTSGVHTFP |
| | lacking C- | AVLQSSGLYSLSSVVTVPSSSLGTQTY |
| | terminal | ICNVNHKPSNTKVDKRVEPKSCDKTHT |
| | lysine | CPPCPAPELLGGPSVFLFPPKPKDTLM |
| | | ISRTPEVTCVVVDVSHEDPEVKFNWYV |
| | | DGVEVHNAKTKPREEQYNSTYRVVSVL |
| | | TVLHQDWLNGKEYKCKVSNKALPAPIE |
| | | KTISKAKGQPREPQVYTLPPSREEMTK |
| | | NQVSLTCLVKGFYPSDIAVEWESNGQP |
| | | ENNYKTTPPVLDSDGSFFLYSKLTVDK |
| | | SRWQQGNVFSCSVMHEALHNHYTQKSL |
| | | SLSPG |
| 98 | IgG1 | ASTKGPSVFPLAPSSKSTSGGTAALGC |
| | (N297A) | LVKDYFPEPVTVSWNSGALTSGVHTFP |
| | | AVLQSSGLYSLSSVVTVPSSSLGTQTY |
| | | ICNVNHKPSNTKVDKRVEPKSCDKTHT |
| | | CPPCPAPELLGGPSVFLFPPKPKDTLM |
| | | ISRTPEVTCVVVDVSHEDPEVKFNWYV |
| | | DGVEVHNAKTKPREEQYASTYRVVSVL |
| | | TVLHQDWLNGKEYKCKVSNKALPAPIE |
| | | KTISKAKGQPREPQVYTLPPSREEMTK |
| | | NQVSLTCLVKGFYPSDIAVEWESNGQP |
| | | ENNYKTTPPVLDSDGSFFLYSKLTVDK |
| | | SRWQQGNVFSCSVMHEALHNHYTQKSL |
| | | SLSPGK |
| 99 | IgG1 | ASTKGPSVFPLAPSSKSTSGGTAALGC |
| | (N297A) | LVKDYFPEPVTVSWNSGALTSGVHTFP |
| | lacking C- | AVLQSSGLYSLSSVVTVPSSSLGTQTY |
| | tcrininal | ICNVNHKPSNTKVDKRVEPKSCDKTHT |
| | lysine | CPPCPAPELLGGPSVFLFPPKPKDTLM |
| | | ISRTPEVTCVVVDVSHEDPEVKFNWYV |
| | | DGVEVHNAKTKPREEQYASTYRVVSVL |
| | | TVLHQDWLNGKEYKCKVSNKALPAPIE |
| | | KTISKAKGQPREPQVYTLPPSREEMTK |
| | | NQVSLTCLVKGFYPSDIAVEWESNGQP |
| | | ENNYKTTPPVLDSDGSFFLYSKLTVDK |
| | | SRWQQGNVFSCSVMHEALHNHYTQKSL |
| | | SLSPG |
| 100 | IgG1 | ASTKGPSVFPLAPSSKSTSGGTAALGC |
| | (D265A/ | LVKDYFPEPVTVSWNSGALTSGVHTFP |
| | P329A) | AVLQSSGLYSLSSVVTVPSSSLGTQTY |
| | | ICNVNHKPSNTKVDKRVEPKSCDKTHT |
| | | CPPCPAPELLGGPSVFLFPPKPKDTLM |
| | | ISRTPEVTCVVVAVSHEDPEVKFNWYV |
| | | DGVEVHNAKTKPREEQYNSTYRVVSVL |
| | | TVLHQDWLNGKEYKCKVSNKALAAPIE |
| | | KTISKAKGQPREPQVYTLPPSREEMTK |
| | | NQVSLTCLVKGFYPSDIAVEWESNGQP |
| | | ENNYKTTPPVLDSD |
| | | GSFFLYSKLTVDKSRWQQGNVFSCSVM |
| | | HEALHNHYTQKSLSLSPGK |
| 101 | IgG1 | ASTKGPSVFPLAPSSKSTSGGTAALGC |
| | (D265A/ | LVKDYFPEPVTVSWNSGALTSGVHTFP |
| | P329A) | AVLQSSGLYSLSSVVTVPSSSLGTQTY |
| | lacking C- | ICNVNHKPSNTKVDKRVEPKSCDKTHT |
| | terminal | CPPCPAPELLGGPSVFLFPPKPKDTLM |
| | lysine | ISRTPEVTCVVVAVSHEDPEVKFNWYV |
| | | DGVEVHNAKTKPREEQYNSTYRVVSVL |
| | | TVLHQDWLNGKEYKCKVSNKALAAPIE |
| | | KTISKAKGQPREPQVYTLPPSREEMTK |
| | | NQVSLTCLVKGFYPSDIAVEWESNGQP |
| | | ENNYKTTPPVLDSDGSFFLYSKLTVDK |
| | | SRWQQGNVFSCSVMHEALHNHYTQKSL |
| | | SLSPG |
| 102 | IgG1 | ASTKGPSVFPLAPSSKSTSGGTAALGC |
| | (L234A/ | LVKDYFPEPVTVSWNSGALTSGVHTFP |
| | L235A) | AVLQSSGLYSLSSVVTVPSSSLGTQTY |
| | | ICNVNHKPSNTKVDKRVEPKSCDKTHT |
| | | CPPCPAPEAAGGPSVFLFPPKPKDTLM |
| | | ISRTPEVTCVVVDVSHEDPEVKFNWYV |
| | | DGVEVHNAKTKPREEQYNSTYRVVSVL |
| | | TVLHQDWLNGKEYKCKVSNKALPAPIE |
| | | KTISKAKGQPREPQVYTLPPSREEMTK |
| | | NQVSLTCLVKGFYPSDIAVEWESNGQP |
| | | ENNYKTTPPVLDSDGSFFLYSKLTVDK |
| | | SRWQQGNVFSCSVMHEALHNHYTQKSL |
| | | SLSPGK |
| 103 | IgG1 | ASTKGPSVFPLAPSSKSTSGGTAALGC |
| | (L234A/ | LVKDYFPEPVTVSWNSGALTSGVHTFP |
| | L235A) | AVLQSSGLYSLSSVVTVPSSSLGTQTY |
| | lacking C- | ICNVNHKPSNTKVDKRVEPKSCDKTHT |
| | terminal | CPPCPAPEAAGGPSVFLFPPKPKDTLM |
| | lysine | ISRTPEVTCVVVDVSHEDPEVKFNWYV |
| | | DGVEVHNAKTKPREEQYNSTYRVVSVL |
| | | TVLHQDWLNGKEYKCKVSNKALPAPIE |
| | | KTISKAKGQPREPQVYTLPPSREEMTK |
| | | NQVSLTCLVKGFYPSDIAVEWESNGQP |
| | | ENNYKTTPPVLDSDGSFFLYSKLTVDK |
| | | SRWQQGNVFSCSVMHEALHNHYTQKSL |
| | | SLSPG |
| 104 | Human | RTVAAPSVFIFPPSDEQLKSGTASVVC |
| | kappa | LLNNFYPREAKVQWKVDNALQSGNSQE |
| | constant | SVTEQDSKDSTYSLSSTLTLSKADYEK |
| | region | HKVYACEVTHQGLSSPVTKSFNRGEC |

TABLE 4
Exemplary sequences of CD73

| SEQ | | |
|---|---|---|
| ID | | |
| NO | Description | Sequence |
| 105 | Human CD73 | MCPRAARAPATLLLALGAVLWP |
| | (Q6NZX3) | AAGAWELTILHTNDVHSRLEQT |
| | | SEDSSKCVNASRCMGGVARLFT |
| | | KVQQIRRAEPNVLLLDAGDQYQ |
| | | GTIWFTVYKGAEVAHFMNALRY |
| | | DAMALGNHEFDNGVEGLIEPLL |
| | | KEAKFPILSANIKAKGPLASQI |
| | | SGLYLPYKVLPVGDEVVGIVGY |
| | | TSKETPFLSNPGTNLVFEDEIT |
| | | ALQPEVDKLKTLNVNKIIALGH |
| | | SGFEMDKLIAQKVRGVDVVVGG |
| | | HSNTFLYTGNPPSKEVPAGKYP |
| | | FIVTSDDGRKVPVVQAYAFGKY |
| | | LGYLKIEFDERGNVISSHGNPI |
| | | LLNSSIPEDPSIKADINKWRIK |
| | | LDNYSTQELGKTIVYLDGSSQS |
| | | CRFRECNMGNLICDAMINNNLR |
| | | HADETFWNHVSMCILNGGGIRS |
| | | PIDERNNGTITWENLAAVLPFG |
| | | GTFDLVQLKGSTLKKAFEHSVH |
| | | RYGQSTGEFLQVGGIHVVYDLS |
| | | RKPGDRVVKLDVLCTKCRVPSY |
| | | DPLKMDEVYKVILPNFLANGGD |
| | | GFQMIKDELLRHDSGDQDINVV |
| | | STYISKMKVIYPAVEGRIKFST |
| | | GSHCHGSFSLIFLSLWAVIFVL |
| | | YQ |
| 106 | Human CD73 | MCPRAARAPATLLLALGAVLWP |
| | isoform | AAGAWELTILHTNDVHSRLEQT |
| | (P21589-1) | SEDSSKCVNASRCMGGVARLFT |
| | | KVQQIRRAEPNVLLLDAGDQYQ |
| | | GTIWFTVYKGAEVAHFMNALRY |
| | | DAMALGNHEFDNGVEGLIEPLL |
| | | KEAKFPILSANIKAKGPLASQI |
| | | SGLYLPYKVLPVGDEVVGIVGY |
| | | TSKETPFLSNPGTNLVFEDEIT |
| | | ALQPEVD |
| | | KLKTLNVNKIIALGHSGFEMDKLI |
| | | AQKVRGVDVVVGGHSNTFLYTGNP |
| | | PSKEVPAGKYPFIVTSDDGRKVPV |
| | | VQAYAFGKYLGYLKIEFDERGNVI |
| | | SSHGNPILLNSSIPEDPSIKADIN |
| | | KWRIKLDNYSTQELGKTIVYLDGS |
| | | SQSCRFRECNMGNLICDAMINNNL |
| | | RHTDEMFWNHVSMCILNGGGIRSP |
| | | IDERNNGTITWENLAAVLPFGGTF |
| | | DLVQLKGSTLKKAFEHSVHRYGQS |
| | | TGEFLQVGGIHVVYDLSRKPGDRV |
| | | VKLDVLCTKCRVPSYDPLKMDEVY |
| | | KVILPNFLANGGDGFQMIKDELLR |
| | | HDSGDQDINVVSTYISKMKVIYPA |
| | | VEGRIKFSTGSHCHGSFSLIFLSL |
| | | WAVIFVLYQ |
| 107 | Human CD73 | MCPRAARAPATLLLALGAVLWPAA |
| | isoform | GAWELTILHTNDVHSRLEQTSEDS |
| | (P21589-2) | SKCVNASRCMGGVARLFTKVQQIR |
| | | RAEPNVLLLDAGDQYQGTIWFTVY |
| | | KGAEVAHFMNALRYDAMALGNHEF |
| | | DNGVEGLIEPLLKEAKFPILSANI |
| | | KAKGPLASQISGLYLPYKVLPVGD |
| | | EVVGIVGYTSKETPFLSNPGTNLV |
| | | FEDEITALQPEVDKLKTLNVNKII |
| | | ALGHSGFEMDKLIAQKVRGVDVVV |
| | | GGHSNTFLYTGNPPSKEVPAGKYP |
| | | FIVTSDDGRKVPVVQAYAFGKYLG |
| | | YLKIEFDERGNVISSHGNPILLNS |
| | | SIPEDPSIKADINKWRIKLDNYST |
| | | QELGKTIVYLDGSSQSCRFRECNM |
| | | GNLICDAMINNNLRHTDEMFWNHV |
| | | SMCILNGGGIRSPIDERNNGIHVV |
| | | YDLSRKPGDRVVKLDVLCTKCRVP |
| | | SYDPLKMDEVYKVILPNFLANGGD |
| | | GFQMIKDELLRHDSGDQDINVVST |
| | | YISKMKVIYPAVEGRIKFSTGSHC |
| | | HGSFSLIFLSLWAVIFVLYQ |
| 108 | CD73 | YLPYKVLPVGDEVVG |
| | fragment | |
| | (residue | |
| | range | |
| | 158-172 | |
| | of SEQ | |
| | ID NO: | |
| | 105) | |
| 109 | CD73 | KLKTLNVNKI |
| | fragment | |
| | (residue | |
| | range | |
| | 206-215 | |
| | of SEQ | |
| | ID NO: | |
| | 105) | |
| 110 | CD73 | MINNNLRHADETFWNHVSMC |
| | fragment | |
| | (residue | |
| | range | |
| | 368-387 | |
| | of SEQ | |
| | ID NO: | |
| | 105) | |
| 121 | CD73 | MINNNLRHTDEMFWNHVSMC |
| | fragment | |
| | (residue | |
| | range | |
| | 368-387 | |
| | of SEQ | |
| | ID NO: | |
| | 106) | |
| 111 | CD73 | YQGTIWFTVYKGAEVAHF |
| | fragment | |
| | (residue | |
| | range | |
| | 87-104 | |
| | of | |
| | SEQ ID | |
| | NO: 105) | |
| 118 | CD73 | RGNVISSHGNPIL |
| | fragment | |
| | (residue | |
| | range | |
| | 297-309 | |
| | of SEQ | |
| | ID NO: | |
| | 105) | |
| 134 | CD73 | WELTILHTNDVHSRLEQTSED |
| | fragment | SSKCVNASRCMGGVARLFTKV |
| | (residue | QQIRRAEPNVLLLDAGDQYQG |
| | range | TIWFTVYKGAEVAHFMNALRY |
| | 27-547 | DAMALGNHEFDNGVEGLIEPL |
| | of SEQ ID | LKEAKFPILSANIKAKGPLAS |
| | NO: 105) | QISGLYLPYKVLPVGDEVVGI |
| | | VGYTSKETPFLSNPGTNLVFE |
| | | DEITALQPEVDKLKTLNVNKI |
| | | IALGHSGFEMDKLIAQKVRGV |
| | | DVVVGGHSNTFLYTGNPPSKE |
| | | VPAGKYPFIVTSDDGRKVPVV |
| | | QAYAFGKYLGYLKIEFDERGN |
| | | VISSHGNPILLNSSIPEDPSI |
| | | KADINKWRIKLDNYSTQELGK |
| | | TIVYLDGSSQSCRFRECNMGN |
| | | LICDAMINNNLRHADETFWNH |
| | | VSMCILNGGGIRSPIDERNNG |
| | | TITWENLAAVLPFGGTFDLVQ |
| | | LKGSTLKKAFEHSVHR |
| | | YGQSTGEFLQVGGIHVVYDLS |
| | | RKPGDRVVKLDVLCTKCRVPS |
| | | YDPLKMDEVYKVILPNFLANG |
| | | GDGFQMIKDELLRHDSGDQDI |
| | | NVVSTYISKMKVIYPAVEGRI |
| | | K |
| 171 | CD73 | WELTILHTNDVHSRLEQTSED |
| | fragment | SSKCVNASRCMGGVARLFTKV |
| | (residue | QQIRRAEPNVLLLDAGDQYQG |
| | range | TIWFTVYKGAEVAHFMNALRY |
| | 27-547 of | DAMALGNHEFDNGVEGLIEPL |
| | SEQ ID | LKEAKFPILSANIKAKGPLAS |
| | NO: 105) | QISGLYLPYKVLPVGDEVVGI |
| | fused to | VGYTSKETPFLSNPGTNLVFE |
| | a | DEITALQPEVDKLKTLNVNKI |
| | C-terminal | IALGHSGFEMDKLIAQKVRGV |
| | 6-His tag | DVVVGGHSNTFLYTGNPPSKE |
| | | VPAGKYPFIVTSDDGRKVPVV |
| | | QAYAFGKYLGYLKIEFDERGN |
| | | VISSHGNPILLNSSIPEDPSI |
| | | KADINKWRIKLDNYSTQELGK |
| | | TIVYLDGSSQSCRFRECNMGN |
| | | LICDAMINNNLRHADETFWNH |
| | | VSMCILNGGGIRSPIDERNNG |
| | | TITWENLAAVLPFGGTFDLVQ |
| | | LKGSTLKKAFEHSVHRYGQST |
| | | GEFLQVGGIHVVYDLSRKPGD |
| | | RVVKLDVLCTKCRVPSYDPLK |
| | | MDEVYKVILPNFLANGGDGFQ |
| | | MIKDELLRHDSGDQDINVVST |
| | | YISKMKVIYPAVEGRIKHHHH |
| | | HH |

In other embodiments, the aforesaid antibody molecules are capable of binding to human CD73 with a dissociation constant (K_{D}) of less than about 1×10⁻⁴ M, 1×10⁻⁵ M, 1×10⁻⁶ M, 1×10⁻⁷ M, 1×10⁻⁸ M, 1×10⁻⁹ M, e.g., as measured by Biacore, Octet, flow cytometry, or ELISA.

In some embodiments, the antibody molecule binds to a mammalian, e g, human or cynomolgus, CD73. For example, the antibody molecule binds to an epitope, e.g., linear or conformational epitope, (e.g., an epitope as described herein), on CD73. In certain aspects, it is advantageous to identify an antibody that binds with high affinity to the human and cynomolgus homologs of a protein of interest. This desirable cross-reactivity allows the same antibody (or two antibodies with the same CDRs or variable regions) to be tested in an animal model and then administered to human patients as a therapeutic.

In some embodiments, disclosed herein is an isolated antibody molecule that competes for binding to human CD73 with the aforesaid anti-CD73 antibody molecules.

In some embodiments, disclosed herein is an isolated antibody molecule that binds to the same epitope as, substantially the same epitope as, an epitope that overlaps with, or an epitope that substantially overlaps with, the epitope of the aforesaid anti-CD73 antibody molecules.

In some embodiments, the antibody molecule binds to the N-terminal domain of human CD73. In some embodiments, the antibody molecule binds to the A-loop and/or B-loop of human CD73. In some embodiments, the antibody molecule, when bound to human CD73, induces a conformational change in the C-loop of human CD73.

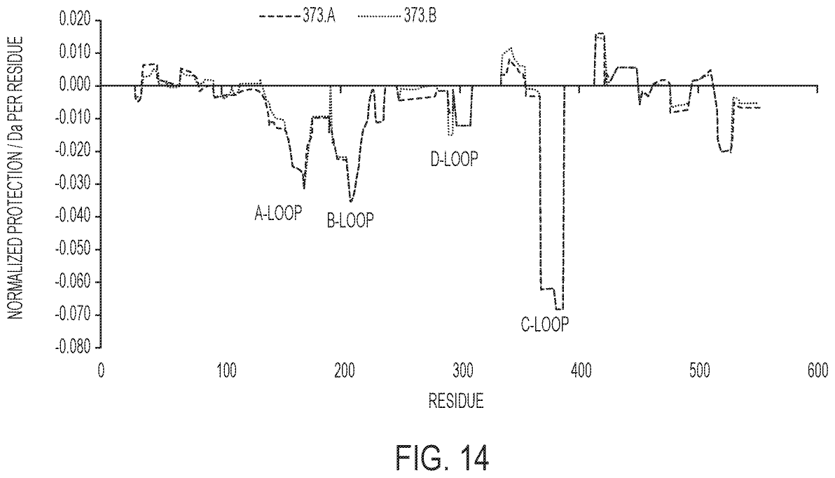

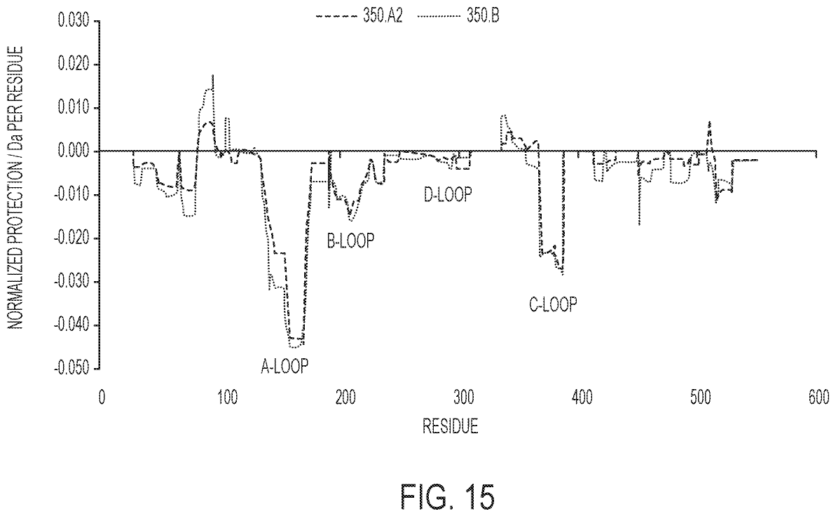

In some embodiments, the antibody molecule reduces hydrogen-deuterium exchange at one or more regions of a protein comprising the amino acid sequence of residues 27-547 of SEQ ID NO: 105 (e.g., a protein consisting of the amino acid sequence of SEQ ID NO: 171) when bound thereto, wherein the one or more regions are selected from the group consisting of residues 158-172, residues 206-215, residues 368-387, and residues 87-104 of SEQ ID NO: 105, e.g., when the antibody molecule is tested as a bivalent antibody molecule using hydrogen deuterium-exchange mass spectrometry, e.g., the method used for FIGS. 14 and 15. Representative data are shown in FIGS. 14 and 15. In some embodiments, hydrogen-deuterium exchange is reduced by at least 10%, 20%, 30%, 40%, 50%, 60%, 70%, 80%, or 90%.

In some embodiments, the antibody molecule reduces hydrogen-deuterium exchange at one or more regions of a protein comprising the amino acid sequence of residues 27-547 of SEQ ID NO: 106 when bound thereto, wherein the one or more regions are selected from the group consisting of residues 158-172, residues 206-215, residues 368-387, and residues 87-104 of SEQ ID NO: 106, e.g., when the antibody molecule is tested as a bivalent antibody molecule using hydrogen deuterium-exchange mass spectrometry.

In some embodiments, the antibody molecule, when bound to a protein comprising the amino acid sequence of residues 27-547 of SEQ ID NO: 105 (e.g., a protein consisting of the amino acid sequence of SEQ ID NO: 171), leads to a greater reduction in hydrogen-deuterium exchange at residues 368-387 of SEQ ID NO: 105 than at residues 297-309 of SEQ ID NO: 105, e.g., as measured by a method described herein, e.g., hydrogen deuterium-exchange mass spectrometry conducted for 1-minute in-exchange at pH 7.5 and room temperature, e.g., the method used for FIGS. 14 and 15. Representative data are shown in FIGS. 14 and 15. In some embodiments, the reduction in hydrogen-deuterium exchange at residues 368-387 of SEQ ID NO: 105 is at least 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, or 4-fold of the reduction in hydrogen-deuterium exchange at residues 297-309 of SEQ ID NO: 105.

In some embodiments, the anti-CD73 antibody molecule, when bound to a protein comprising the amino acid sequence of residues 27-547 of SEQ ID NO: 106, leads to a greater reduction in hydrogen-deuterium exchange at residues 368-387 of SEQ ID NO: 106 than at residues 297-309 of SEQ ID NO: 106, e.g., as measured by a method described herein, e.g., hydrogen deuterium-exchange mass spectrometry conducted for 1-minute in-exchange at pH 7.5 and room temperature.

In some embodiments, the anti-CD73 antibody molecule, when bound to human CD73, reduces hydrogen-deuterium exchange at the C-loop of human CD73 to a greater extent than at the A-loop, B-loop, or D-loop of human CD73, e.g., as measured by a method described herein, e.g., hydrogen deuterium-exchange mass spectrometry conducted for 1-minute in-exchange at pH 7.5 and room temperature, e.g., the method used for FIGS. 14 and 15. Representative data are shown in FIGS. 14 and 15. In some embodiments, the reduction in hydrogen-deuterium exchange at the C-loop of human CD73 is at least 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, or 4-fold of the reduction in hydrogen-deuterium exchange at the A-loop, B-loop, or D-loop of human CD73.

In some embodiments, the anti-CD73 antibody molecule, when bound to a protein comprising the amino acid sequence of residues 27-547 of SEQ ID NO: 105 (e.g., a protein consisting of the amino acid sequence of SEQ ID NO: 171), reduces hydrogen-deuterium exchange at residues 368-387 of SEQ ID NO: 105 to a greater extent than at residues 158-172, residues 206-215, or residues 297-309 of SEQ ID NO: 105, e.g., as measured by a method described herein, e.g., hydrogen deuterium-exchange mass spectrometry conducted for 1-minute in-exchange at pH 7.5 and room temperature, e.g., the method used for FIGS. 14 and 15. Representative data are shown in FIGS. 14 and 15. In some embodiments, the reduction in hydrogen-deuterium exchange at residues 368-387 of SEQ ID NO: 105 is at least 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, or 4-fold of the reduction in hydrogen-deuterium exchange at residues 158-172, residues 206-215, or residues 297-309 of SEQ ID NO: 105.

In some embodiments, the anti-CD73 antibody molecule, when bound to a protein comprising the amino acid sequence of residues 27-547 of SEQ ID NO: 106, reduces hydrogen-deuterium exchange at residues 368-387 of SEQ ID NO: 106 to a greater extent than at residues 158-172, residues 206-215, or residues 297-309 of SEQ ID NO: 106, e.g., as measured by a method described herein, e.g., hydrogen deuterium-exchange mass spectrometry conducted for 1-minute in-exchange at pH 7.5 and room temperature.

In some embodiments, the anti-CD73 antibody molecule, when bound to human CD73, reduces hydrogen-deuterium exchange at the A-loop of human CD73 to a greater extent than at the B-loop, C-loop, or D-loop of human CD73, e.g., as measured by a method described herein, e.g., hydrogen deuterium-exchange mass spectrometry conducted for 1-minute in-exchange at pH 7.5 and room temperature, e.g., the method used for FIGS. 14 and 15. Representative data are shown in FIGS. 14 and 15. In some embodiments, the reduction in hydrogen-deuterium exchange at the A-loop of human CD73 is at least 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, or 4-fold of the reduction in hydrogen-deuterium exchange at the B-loop, C-loop, or D-loop of human CD73.

In some embodiments, the anti-CD73 antibody molecule, when bound to a protein comprising the amino acid sequence of residues 27-547 of SEQ ID NO: 105 (e.g., a protein consisting of the amino acid sequence of SEQ ID NO: 171), reduces hydrogen-deuterium exchange at residues 158-172 of SEQ ID NO: 105 to a greater extent than at residues 206-215, residues 368-387, or residues 297-309 of SEQ ID NO: 105, e.g., as measured by a method described herein, e.g., hydrogen deuterium-exchange mass spectrometry conducted for 1-minute in-exchange at pH 7.5 and room temperature, e.g., the method used for FIGS. 14 and 15. Representative data are shown in FIGS. 14 and 15. In some embodiments, the reduction in hydrogen-deuterium exchange at residues 158-172 of SEQ ID NO: 105 is at least 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, or 4-fold of the reduction in hydrogen-deuterium exchange at residues 206-215, residues 368-387, or residues 297-309 of SEQ ID NO: 105.

In some embodiments, the anti-CD73 antibody molecule, when bound to a protein comprising the amino acid sequence of residues 27-547 of SEQ ID NO: 105 (e.g., a protein consisting of the amino acid sequence of SEQ ID NO: 171), reduces hydrogen-deuterium exchange at residues 158-172 of SEQ ID NO: 106 to a greater extent than at residues 206-215, residues 368-387, or residues 297-309 of SEQ ID NO: 106, e.g., as measured by a method described herein, e.g., hydrogen deuterium-exchange mass spectrometry conducted for 1-minute in-exchange at pH 7.5 and room temperature.

In some embodiments, the anti-CD73 antibody molecule, when bound to a protein comprising the amino acid sequence of residues 27-547 of SEQ ID NO: 105 (e.g., a protein consisting of the amino acid sequence of SEQ ID NO: 171), is capable of reducing hydrogen-deuterium exchange at one or more regions of human CD73, wherein the one or more regions are selected from the group consisting of residues 158-172, residues 206-215, residues 368-387, and residues 87-104 of SEQ ID NO: 105, wherein the region having the greatest reduction among the one or more regions is not residues 206-215 of SEQ ID NO: 105, e.g., as measured by a method described herein, e.g., hydrogen deuterium-exchange mass spectrometry conducted for 1-minute in-exchange at pH 7.5 and room temperature, e.g., the method used for FIGS. 14 and 15. Representative data are shown in FIGS. 14 and 15.

In some embodiments, the anti-CD73 antibody molecule, when bound to a protein comprising the amino acid sequence of residues 27-547 of SEQ ID NO: 106, is capable of reducing hydrogen-deuterium exchange at one or more regions of human CD73, wherein the one or more regions are selected from the group consisting of residues 158-172, residues 206-215, residues 368-387, and residues 87-104 of SEQ ID NO: 106, wherein the region having the greatest reduction among the one or more regions is not residues 206-215 of SEQ ID NO: 106, e.g., as measured by a method described herein, e.g., hydrogen deuterium-exchange mass spectrometry conducted for 1-minute in-exchange at pH 7.5 and room temperature.

In some embodiments, the anti-CD73 antibody molecule, when bound to a protein comprising the amino acid sequence of residues 27-547 of SEQ ID NO: 105 (e.g., a protein consisting of the amino acid sequence of SEQ ID NO: 171), leads to a reduction in hydrogen-deuterium exchange of more than, e.g., 0.02, 0.03, 0.04, 0.05, or 0.06 Da per residue at residues 368-387 of SEQ ID NO: 105, e.g., as measured by a method described herein, e.g., hydrogen deuterium-exchange mass spectrometry conducted for 1-minute in-exchange at pH 7.5 and room temperature, e.g., the method used for FIGS. 14 and 15. Representative data are shown in FIGS. 14 and 15.

In some embodiments, the anti-CD73 antibody molecule, when bound to a protein comprising the amino acid sequence of residues 27-547 of SEQ ID NO: 105 (e.g., a protein consisting of the amino acid sequence of SEQ ID NO: 171), leads to a reduction in hydrogen-deuterium exchange of less than, e.g., 0.05, 0.04, 0.03, or 0.02 Da per residue at residues 206-215 of SEQ ID NO: 105, e.g., as measured by a method described herein, e.g., hydrogen deuterium-exchange mass spectrometry conducted for 1-minute in-exchange at pH 7.5 and room temperature, e.g., the method used for FIGS. 14 and 15. Representative data are shown in FIGS. 14 and 15.

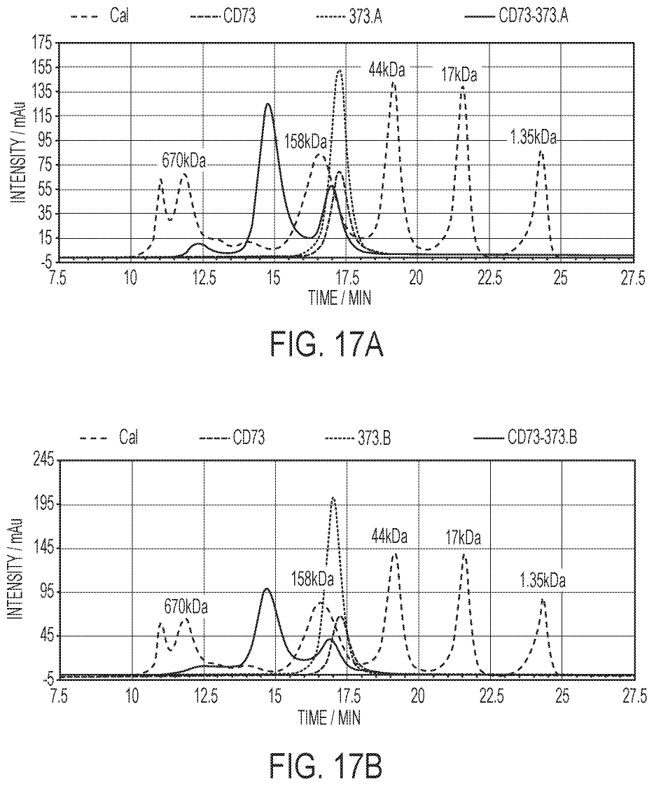

In some embodiments, the antibody molecule that binds to a human CD73 dimer, said dimer consisting of a first CD73 monomer and a second CD73 monomer, wherein when the antibody molecule comprises a first antigen binding domain and a second antigen binding domain, the first antigen binding domain binds to the first CD73 monomer and the second antigen binding domain binds to the second CD73 monomer, e.g., when tested using size exclusion chromatography, e.g., when tested using a method described herein, e.g., the method used for FIGS. 17A and 17B.

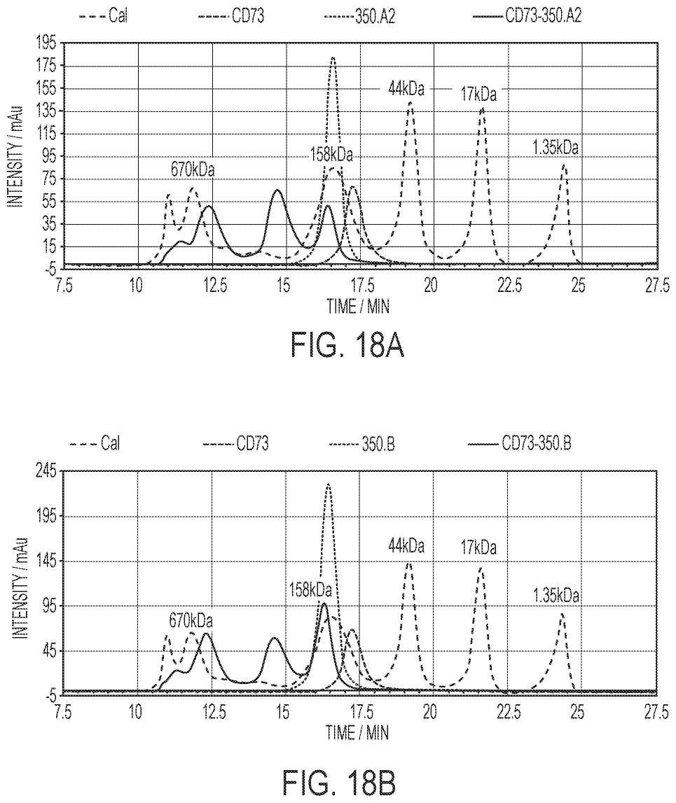

In some embodiments, disclosed herein is a composition comprising a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein when the antibody molecules in the plurality each comprises the same first antigen binding domain and the same second antigen binding domain, at least 30%, 35%, 40%, 45%, 50%, 55%, 60%, 65%, 70%, 75%, or 80% of the antibody molecules in said composition bind to the CD73 dimer to form a complex, wherein each of said complex consists of one antibody molecule and one CD73 dimer, e.g., when measured using size exclusion chromatography and the percentage value is obtained by determining the amount of the antibody molecules in the complex relative to the total amount of the antibody molecules binding to CD73 (excluding unbound antibody molecules), e.g. when tested using a method described herein, e.g., the method used for FIGS. 17A, 17B, 18A, and 18B. Representative data are shown in FIGS. 17A, 17B, 18A, and 18B.

In some embodiments, disclosed herein is a plurality of antibody molecules that bind to a human CD73 dimer, said dimer consisting of a first CD73 monomer and a second CD73 monomer, each monomer comprising the amino acid sequence of residues 27-547 of SEQ ID NO: 105 (e.g., each monomer consisting of the amino acid sequence of SEQ ID NO: 171), wherein when the antibody molecules in the plurality each comprises the same first antigen binding domain and the same second antigen binding domain, at most 20%, 25%, 30%, 35%, 40%, 45%, 50%, 55%, 60%, 65%, or 70% of the antibody molecules in said composition bind to the CD73 dimer to form a complex, wherein each of said complex comprises two or more antibody molecules and two or more CD73 dimers, e.g., when measured using size exclusion chromatography and the percentage value is obtained by determining the amount of the antibody molecules in the complex relative to the total amount of the antibody molecules binding to CD73 (excluding unbound antibody molecules), e.g. when tested using a method described herein, e.g., the method used for FIGS. 17A, 17B, 18A, and 18B. Representative data are shown in FIGS. 17A, 17B, 18A, and 18B.

In some embodiments, the anti-CD73 antibody molecule, upon binding to human CD73, does not lead to oligomerization of human CD73.

In some embodiments, the antibody molecule preferentially binds to an open conformation, e.g., a catalytically inactive conformation, of CD73 over a closed conformation, e.g., a catalytically active conformation, of CD73, e.g., does not bind to or binds to the closed conformation, e.g., the catalytically active conformation, of CD73 with lower affinity, e.g., 50%, 60%, 70%, 80%, 90%, 95%, or 99% lower affinity than when the antibody molecule binds to the open conformation, e.g., the catalytically inactive conformation, of CD73.

In some embodiments, the anti-CD73 antibody molecule prevents or reduces the conversion of human CD73 from a catalytically inactive open conformation to a catalytically active closed conformation. In some embodiments, the conversion of human CD73 from a catalytically inactive open conformation to a catalytically active closed conformation is reduced by at least 1.5-fold, 2-fold, 5-fold, 10-fold, 20-fold, 30-fold, 40-fold, 50-fold, 60-fold, 70-fold, 80-fold, 90-fold, or 100-fold, compared to the conversion in the absence of the antibody molecule.

In some embodiments, the anti-CD73 antibody molecule contacts, e.g., directly or indirectly, at least one residue within residues 158-172 of SEQ ID NO: 105. In some embodiments, the anti-CD73 antibody molecule contacts, e.g., directly or indirectly, at least one residue within residues 206-215 of SEQ ID NO: 105. In some embodiments, the anti-CD73 antibody molecule contacts, e.g., directly or indirectly, at least one residue within residues 368-387 of SEQ ID NO: 105. In some embodiments, the anti-CD73 antibody molecule contacts, e.g., directly or indirectly, at least one residue within residues 87-104 of SEQ ID NO: 105. In some embodiments, the anti-CD73 antibody molecule contacts, e.g., directly or indirectly, at least one residue within residues 368-387 of SEQ ID NO: 106.

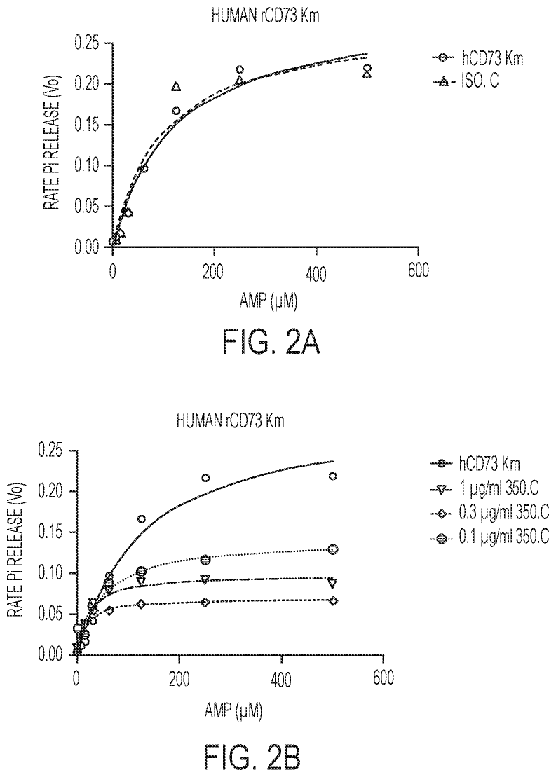

In some embodiments, the anti-CD73 antibody molecule inhibits or reduces the enzymatic activity of CD73 (e.g., soluble human CD73 or membrane-bound human CD73), e.g., human CD73 mediated conversion of adenosine monophosphate (AMP) to adenosine, e.g., as measured by a method described herein, e.g., a malachite green (MG) phosphate assay or a modified Cell Titer Glo (CTG) assay, e.g., the method used in FIG. 2B, 3A, 3B, 3C, 4, 5, 6, 7A, 7B, 7C, 8A, 8B, 9A, 9B, 10, 11A, 11B, or 13. Representative data are shown in FIGS. 2B, 3A, 3B, 3C, 4, 5, 6, 7A, 7B, 7C, 8A, 8B, 9A, 9B, 10, 11A, 11B, and 13. In some embodiments, the enzymatic activity of CD73 (e.g., soluble human CD73 or membrane-bound human CD73) is reduced by at least 10%, 20%, 30%, 40%, 50%, 60%, 70%, 80%, 90%, or 99%.

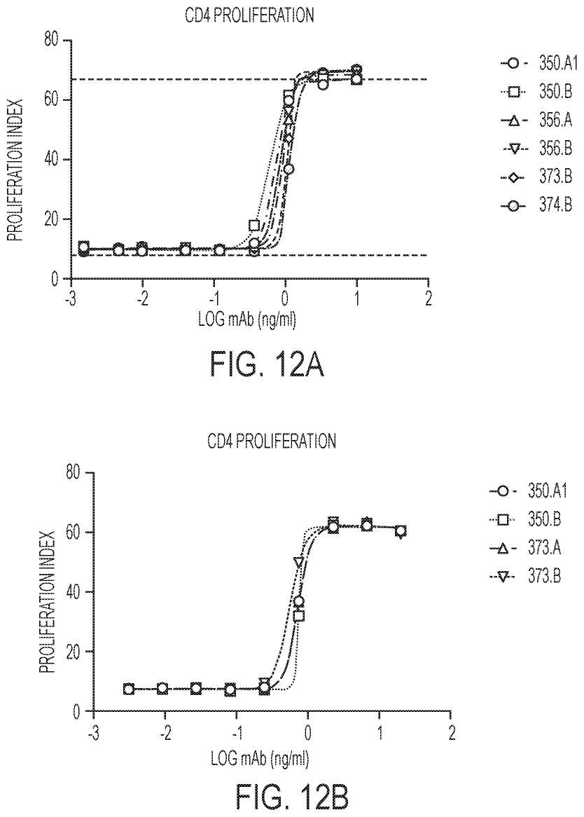

In some embodiments, the anti-CD73 antibody molecule increases proliferation of anti-CD3/anti-CD28 stimulated T cells, e.g., CD4+ T cells, in the presence of adenosine monophosphate (AMP), e.g., as measured by a method described herein, e.g., a CellTrace Violet (CTV) cell proliferation assay, e.g., the method used in FIG. 12A or 12B. Representative data are shown in FIGS. 12A and 12B. In some embodiments, anti-CD3/anti-CD28 stimulated T cell, e.g., CD4+ T cell, proliferation is increased by at least 2-fold, 3-fold, 4-fold, 5-fold, 6-fold, 7-fold, 8-fold, 9-fold, or 10-fold.

In another aspect, the invention provides an isolated nucleic acid encoding any of the aforesaid antibody molecules, vectors and host cells thereof. The nucleic acid molecule includes but is not limited to RNA, genomic DNA and cDNA.

In some embodiments, the isolated nucleic acid encodes the antibody heavy chain variable region, light chain variable region, heavy chain, and/or light chain of any of the aforesaid antibody molecules.

In some embodiments, the isolated nucleic acid encodes a heavy chain variable region, wherein the nucleic acid comprises the nucleotide sequence of SEQ ID NO: 45, 78, 85, 143, 152, 160, 67, 32, 11, or 169, or a nucleotide sequence having at least about 85%, 90%, 95%, or 99% sequence identity to SEQ ID NO: 45, 78, 85, 143, 152, 160, 67, 32, 11, or 169.

In some embodiments, the isolated nucleic acid encodes a heavy chain, wherein the nucleic acid comprises the nucleotide sequence of SEQ ID NO: 47, 80, 87, 69, 34, or 13, or a nucleotide sequence having at least about 85%, 90%, 95%, or 99% sequence identity to SEQ ID NO: 47, 80, 87, 69, 34, or 13.

In some embodiments, the isolated nucleic acid encodes a light chain variable region, wherein the nucleic acid comprises the nucleotide sequence of SEQ ID NO: 56, 144, 22, or 170, or a nucleotide sequence having at least about 85%, 90%, 95%, or 99% sequence identity to SEQ ID NO: 56, 144, 22, or 170.

In some embodiments, the isolated nucleic acid encodes a light chain, wherein the nucleic acid comprises the nucleotide sequence of SEQ ID NO: 58 or 24, or a nucleotide sequence having at least about 85%, 90%, 95%, or 99% sequence identity to SEQ ID NO: 58 or 24.

### Pharmaceutical Compositions and Kits

In some aspects, this disclosure provides compositions, e.g., pharmaceutically acceptable compositions, which include an anti-CD73 antibody molecule described herein, formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g. by injection or infusion).

The compositions set out herein may be in a variety of forms. These include, for example, liquid, semi-solid and solid dosage forms, such as liquid solutions (e.g., injectable and infusible solutions), dispersions or suspensions, liposomes, and suppositories. A suitable form depends on the intended mode of administration and therapeutic application. Typical suitable compositions are in the form of injectable or infusible solutions. One suitable mode of administration is parenteral (e.g., intravenous, subcutaneous, intraperitoneal, intramuscular). In some embodiments, the antibody molecule is administered by intravenous infusion or injection. In certain embodiments, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibody molecules can be administered by a variety of methods. Several are known in the art, and for many therapeutic applications, an appropriate route/mode of administration is intravenous injection or infusion. In an embodiment, the antibody molecules can be administered by intravenous infusion at a rate of more than 20 mg/min, e.g., 20-40 mg/min. In an embodiment, the antibody molecules can be administered by intravenous infusion at a rate of greater than or equal to 40 mg/min to reach a dose of about 35 to 440 mg/m², about 70 to 310 mg/m², or about 110 to 130 mg/m². In an embodiment, the antibody molecules can be administered by intravenous infusion at a rate of less than 10 mg/min, e.g., less than or equal to 5 mg/min to reach a dose of about 1 to 100 mg/m², about 5 to 50 mg/m², about 7 to 25 mg/m², or about 10 mg/m². As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody molecule can be orally administered, for example, with an inert diluent or an assimilable edible carrier. The antibody molecule (and other ingredients, if desired) may also be enclosed in a hard or soft shell gelatin capsule, compressed into tablets, or incorporated directly into the subject's diet. For oral therapeutic administration, the antibody molecule may be incorporated with excipients and used in the form of ingestible tablets, buccal tablets, troches, capsules, elixirs, suspensions, syrups, wafers, and the like. To administer an antibody molecule by other than parenteral administration, it may be necessary to coat the compound with, or co-administer the compound with, a material to prevent its inactivation. Therapeutic compositions can also be administered with medical devices known in the art.

Dosage regimens are adjusted to provide the desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

In some embodiments, an anti-CD73 antibody molecule disclosed herein is administered by injection (e.g., subcutaneously or intravenously) at a dose (e.g., a flat dose) of about 60 mg to 2400 mg, e.g., about 100 mg to 2400 mg, about 100 mg to 2200 mg, about 100 mg to 2000 mg, about 100 mg to 1800 mg, about 100 mg to 1600 mg, about 100 mg to 1400 mg, about 100 mg to 1200 mg, about 100 mg to 1000 mg, about 100 mg to 800 mg, about 100 mg to 600 mg, about 100 mg to 400 mg, about 100 mg to 200 mg, or about 100 mg, about 180 mg, or about 200 mg. The dosing schedule (e.g., flat dosing schedule) can vary from e.g., once a week to once every 2, 3, or 4 weeks. In one embodiment, an anti-CD73 antibody molecule disclosed herein is administered at a dose from about 100 mg to 200 mg once every week, once every two weeks, or once every three weeks. In one embodiment, an anti-CD73 antibody molecule disclosed herein is administered at a dose of at least about 180 mg once every two weeks.

In some embodiments, an anti-CD73 antibody molecule disclosed herein is administered by injection (e.g., subcutaneously or intravenously) at a dose (e.g., a flat dose) of about 5 mg to 100 mg, about 100 mg to 500 mg, about 500 mg to 1000 mg, about 1000 mg to 1500 mg, about 1500 mg to 2000 mg, about 2000 mg to 2500 mg, about 2500 mg to 3000 mg, about 3000 mg to 3500 mg, or about 3500 mg to 4000 mg, e.g., once every week (QW), once every two weeks (Q2W), or once every four weeks (Q4W). In certain embodiments, the antibody molecule is administered, e.g., intravenously, at a dose of about 6 mg, about 20 mg, about 60 mg, about 200 mg, about 600 mg, about 1200 mg, about 2400 mg, about 3000 mg, or about 3600 mg, e.g., QW, Q2W, or Q4W. In certain embodiments, the antibody molecule is administered, e.g., intravenously, at a dose of about 60 mg Q2W. In certain embodiments, the antibody molecule is administered, e.g., intravenously, at a dose of about 200 mg Q2W. In certain embodiments, the antibody molecule is administered, e.g., intravenously, at a dose of about 600 mg Q2W. In certain embodiments, the antibody molecule is administered, e.g., intravenously, at a dose of about 1200 mg Q2W. In certain embodiments, the antibody molecule is administered, e.g., intravenously, at a dose of about 2400 mg Q2W. In certain embodiments, the antibody molecule is administered, e.g., intravenously, at a dose of about 3000 mg Q2W. In certain embodiments, the antibody molecule is administered, e.g., intravenously, at a dose of about 3600 mg Q2W.

In one embodiment, an anti-CD73 antibody molecule disclosed herein is administered, e.g., by infusion, over a period of 30 minutes, a period of 1 hour, or a period of up to 2 hours.

An exemplary, non-limiting range for a therapeutically or prophylactically effective amount of an antibody molecule is 0.1-30 mg/kg, more preferably 1-25 mg/kg. Dosages and therapeutic regimens of the anti-CD73 antibody molecule can be determined by a skilled artisan. In certain embodiments, the anti-CD73 antibody molecule is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks. In one embodiment, the anti-CD73 antibody molecule is administered at a dose from about 10 to 20 mg/kg every other week. The antibody molecule can be administered by intravenous infusion at a rate of more than 20 mg/min, e.g., 20-40 mg/min, e.g., greater than or equal to 40 mg/min to reach a dose of about 35 to 440 mg/m², about 70 to 310 mg/m², or about 110 to 130 mg/m². In embodiments, the infusion rate of about 110 to 130 mg/m² achieves a level of about 3 mg/kg. In other embodiments, the antibody molecule can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², or about 10 mg/m². In some embodiments, the antibody is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include a “therapeutically effective amount” or a “prophylactically effective amount” of an antibody molecule of the invention. A “therapeutically effective amount” refers to an amount effective, at dosages and for periods of time necessary, to achieve the desired therapeutic result. A therapeutically effective amount of the modified antibody or antibody fragment may vary according to factors such as the disease state, age, sex, and weight of the individual, and the ability of the antibody or antibody portion to elicit a desired response in the individual. A therapeutically effective amount is also one in which any toxic or detrimental effects of the antibody molecule are outweighed by the therapeutically beneficial effects. A “therapeutically effective dosage” preferably inhibits a measurable parameter by at least about 20%, more preferably by at least about 40%, even more preferably by at least about 60%, and still more preferably by at least about 80% relative to untreated subjects. The measurable parameter may be, e.g., tumor growth rate or pathogen growth rate. The ability of an antibody molecule to inhibit a measurable parameter can be evaluated in an animal model system predictive of efficacy in the corresponding human disease. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the invention is a kit comprising an antibody molecule described herein. The kit can include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody molecule for administration; pharmaceutically acceptable carriers; and devices or other materials for administration to a subject.

### Uses of Anti-CD73 Antibody Molecules

The anti-CD73 antibody molecules disclosed herein have in vitro and in vivo diagnostic, as well as therapeutic and prophylactic utilities. For example, these molecules can be administered to cells in culture, in vitro or ex vivo, or to a subject, e.g., a human subject, to treat, prevent, and/or diagnose a variety of disorders, such as cancers and infectious disorders.

Accordingly, in one aspect, the invention provides a method of modifying an immune response in a subject comprising administering to the subject the antibody molecule described herein, such that the immune response in the subject is modified. In one embodiment, the immune response is enhanced, stimulated or up-regulated. In one embodiment, the antibody molecules enhance an immune response in a subject by blockade of CD73.

As used herein, the term “subject” is intended to include human and non-human animals. In one embodiment, the subject is a human subject, e.g., a human patient having a disorder or condition characterized by abnormal CD73 functioning. The term “non-human animals” includes mammals and non-mammals, such as non-human primates. In one embodiment, the subject is a human. In one embodiment, the subject is a human patient in need of enhancement of an immune response. In one embodiment, the subject is immunocompromised, e.g., the subject is undergoing, or has undergone a chemotherapeutic or radiation therapy. Alternatively, or in combination, the subject is, or is at risk of being, immunocompromised as a result of an infection. The methods and compositions described herein are suitable for treating human patients having a disorder that can be treated by augmenting the T-cell mediated immune response. For example, the methods and compositions described herein can enhance a number of immune activities. In one embodiment, the subject has increased number or activity of tumour-infiltrating T lymphocytes (TILs).

### Therapeutic Uses

### Cancer

In one aspect, the invention relates to treatment of a subject in vivo using an anti-CD73 antibody molecule such that growth of cancerous tumors is inhibited or reduced. An anti-CD73 antibody may be used alone to inhibit the growth of cancerous tumors. Alternatively, an anti-CD73 antibody may be used in combination with one or more of: a standard of care treatment (e.g., for cancers), another antibody molecule, an immunomodulator (e.g., an activator of a costimulatory molecule or an inhibitor of a coinhibitory molecule); a vaccine, e.g., a therapeutic cancer vaccine; or other forms of cell therapy, as described below.

Accordingly, in one embodiment, the invention provides a method of inhibiting growth of tumor cells in a subject, comprising administering to the subject a therapeutically effective amount of an anti-CD73 antibody molecule described herein.

In one embodiment, the methods are suitable for the treatment of cancer in vivo. To achieve antigen-specific enhancement of immunity, the anti-CD73 antibody molecule can be administered together with an antigen of interest. When antibodies to CD73 are administered in combination with one or more agents, the combination can be administered in either order or simultaneously.

### Types of Cancer

In another aspect, a method of treating a subject, e.g., reducing or ameliorating, a hyperproliferative condition or disorder (e.g., a cancer), e.g., solid tumor, a hematological cancer, soft tissue tumor, or a metastatic lesion, in a subject is provided. The method includes administering to the subject one or more anti-CD73 antibody molecules described herein, alone or in combination with other agents or therapeutic modalities.

As used herein, the term “cancer” is meant to include all types of cancerous growths or oncogenic processes, metastatic tissues or malignantly transformed cells, tissues, or organs, irrespective of histopathologic type or stage of invasiveness. Examples of cancerous disorders include, but are not limited to, solid tumors, hematological cancers, soft tissue tumors, and metastatic lesions. Examples of solid tumors include malignancies, e.g., sarcomas, and carcinomas (including adenocarcinomas and squamous cell carcinomas), of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. Squamous cell carcinomas include malignancies, e.g., in the lung, esophagus, skin, head and neck region, oral cavity, anus, and cervix. In one embodiment, the cancer is a melanoma, e.g., an advanced stage melanoma. Metastatic lesions of the aforementioned cancers can also be treated or prevented using the methods and compositions of the invention.

Exemplary cancers whose growth can be inhibited using the antibodies molecules disclosed herein include cancers typically responsive to immunotherapy. Non-limiting examples of preferred cancers for treatment include melanoma (e.g., metastatic malignant melanoma), renal cancer (e.g., clear cell carcinoma), prostate cancer (e.g., hormone refractory prostate adenocarcinoma), breast cancer, colon cancer and lung cancer (e.g., non-small cell lung cancer). Additionally, refractory or recurrent malignancies can be treated using the antibody molecules described herein.

Examples of other cancers that can be treated include bone cancer, pancreatic cancer, skin cancer, cancer of the head or neck, cutaneous or intraocular malignant melanoma, uterine cancer, ovarian cancer, rectal cancer, anal cancer, gastro-esophageal, stomach cancer, testicular cancer, uterine cancer, carcinoma of the fallopian tubes, carcinoma of the endometrium, carcinoma of the cervix, carcinoma of the vagina, carcinoma of the vulva, Merkel cell cancer, Hodgkin lymphoma, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, multiple myeloma, myelodisplastic syndromes, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos (e.g., mesothelioma), and combinations of said cancers.

In some embodiments, the therapies here can be used to treat a patient that has (or is identified as having) a cancer associated with an infection, e.g., a viral or bacterial infection. Exemplary cancers include cervical cancer, anal cancer, HPV-associated head and neck squamous cell cancer, HPV-associated esophageal papillomas, HHV6-associated lymphomas, EBV-associated lymphomas (including Burkitt lymphoma), Gastric MALT lymphoma, other infection-associated MALT lymphomas, HCC, and Kaposi's sarcoma.

In other embodiments, the cancer is a hematological malignancy or cancer including but is not limited to a leukemia or a lymphoma. For example, the anti-CD73 antibody molecule can be used to treat cancers and malignancies including, but not limited to, e.g., acute leukemias including but not limited to, e.g., B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); additional hematologic cancers or hematologic conditions including, but not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like.

In one embodiment, the cancer is chosen from a lung cancer (e.g., a non-small cell lung cancer

(NSCLC) (e.g., a NSCLC with squamous and/or non-squamous histology, or a NSCLC adenocarcinoma)), a melanoma (e.g., an advanced melanoma), a renal cancer (e.g., a renal cell carcinoma, e.g., clear cell renal cell carcinoma), a liver cancer, a myeloma (e.g., a multiple myeloma), a prostate cancer, a breast cancer (e.g., a breast cancer that does not express one, two or all of estrogen receptor, progesterone receptor, or Her2/neu, e.g., a triple negative breast cancer), a colorectal cancer (e.g., microsatellite stable (MSS) colorectal cancer), ovarian cancer, a pancreatic cancer, a head and neck cancer (e.g., head and neck squamous cell carcinoma (HNSCC), anal cancer, gastro-esophageal cancer, thyroid cancer, cervical cancer, a lymphoproliferative disease (e.g., a post-transplant lymphoproliferative disease) or a hematological cancer, T-cell lymphoma, a non-Hogdkin's lymphoma, or a leukemia (e.g., a myeloid leukemia).

In one embodiment, the cancer is chosen from lung cancer (e.g., non-small cell lung cancer), pancreas cancer (e.g., pancreatic ductal adenocarcinoma), breast cancer (e.g., triple-negative breast cancer), melanoma, head and neck cancer (e.g., squamous head and neck cancer), colorectal cancer (e.g., microsatellite stable (MSS) colorectal cancer), ovarian cancer, or renal cancer (e.g., renal cell carcinoma). In one embodiment, the cancer is chosen from bladder cancer, leukemia, lymphoma, glioma, glioblastoma, ovarian cancer, thyroid cancer, esophageal cancer, prostate cancer, uterine/cervical cancer, testicular cancer, esophageal cancer, gastrointestinal cancer, colon cancer, kidney cancer, stomach cancer, germ cell cancer, bone cancer, liver cancer, skin cancer, neoplasm of the central nervous system, myeloma, sarcoma, and virus-related cancer.

### Combination of Anti-CD73 Antibody Molecules

The anti-CD73 antibody molecules can be used in combination with other therapies. For example, the combination therapy can include a composition of the present invention co-formulated with, and/or co-administered with, one or more additional therapeutic agents, e.g., one or more anti-cancer agents, cytotoxic or cytostatic agents, hormone treatment, vaccines, and/or other immunotherapies. In other embodiments, the antibody molecules are administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

By “in combination with,” it is not intended to imply that the therapy or the therapeutic agents must be administered at the same time and/or formulated for delivery together, although these methods of delivery are within the scope described herein. The anti-CD73 antibody molecules can be administered concurrently with, prior to, or subsequent to, one or more other additional therapies or therapeutic agents. The anti-CD73 antibody molecule and the other agent or therapeutic protocol can be administered in any order. In general, each agent will be administered at a dose and/or on a time schedule determined for that agent. In will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

### Exemplary Adenosine A2A Receptor Antagonists

In certain embodiments, the anti-CD73 molecules described herein are administered in combination with an adenosine A2A receptor (A2AR) antagonist. Exemplary A2AR antagonists include, e.g., PBF509 (Palobiofarma/Novartis), CPI444/V81444 (Corvus/Genentech), AZD4635/HTL-1071 (AstraZeneca/Heptares), Vipadenant (Redox/Juno), GBV-2034 (Globavir), AB928 (Arcus Biosciences), Theophylline, Istradefylline (Kyowa Hakko Kogyo), Tozadenant/SYN-115 (Acorda), KW-6356 (Kyowa Hakko Kogyo), ST-4206 (Leadiant Biosciences), and Preladenant/SCH 420814 (Merck/Schering).

In certain embodiments, the A2AR antagonist is PBF509. PBF509 and other A2AR antagonists are disclosed in U.S. Pat. No. 8,796,284 and WO 2017/025918, herein incorporated by reference in their entirety. PBF509 refers to 5-bromo-2,6-di-(1H-pyrazol-1-yl)pyrimidine-4-amine with the following structure:

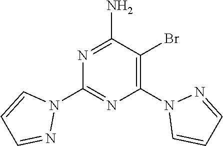 [SMILES: Nc1nc(-n2cccn2)nc(-n2cccn2)c1Br]

In certain embodiments, the A2AR antagonist is CPI444/V81444. CPI-444 and other A2AR antagonists are disclosed in WO 2009/156737, herein incorporated by reference in its entirety. In certain embodiments, the A2AR antagonist is (S)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine In certain embodiments, the A2AR antagonist is (R)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine, or racemate thereof. In certain embodiments, the A2AR antagonist is 7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine In certain embodiments, the A2AR antagonist has the following structure:

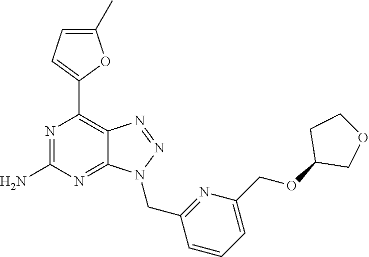 [SMILES: Cc1ccc(-c2nc(N)nc3c2nnn3Cc2cccc(CO[C@H]3CCOC3)n2)o1]

In certain embodiments, the A2AR antagonist is AZD4635/HTL-1071. A2AR antagonists are disclosed in WO 2011/095625, herein incorporated by reference in its entirety. In certain embodiments, the A2AR antagonist is 6-(2-chloro-6-methylpyridin-4-yl)-5-(4-fluorophenyl)-1,2,4-triazin-3-amine In certain embodiments, the A2AR antagonist has the following structure:

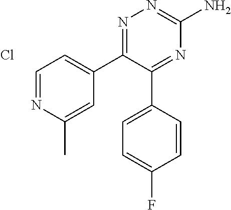 [SMILES: Cl ; Cc1cc(-c2nnc(N)nc2-c2ccc(F)cc2)ccn1]

In certain embodiments, the A2AR antagonist is ST-4206 (Leadiant Biosciences). In certain embodiments, the A2AR antagonist is an A2AR antagonist described in U.S. Pat. No. 9,133,197, herein incorporated by reference in its entirety. In certain embodiments, the A2AR antagonist has the following structure:

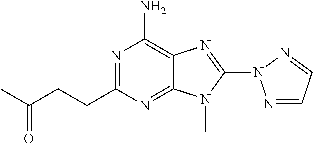 [SMILES: CC(=O)CCc1nc(N)c2nc(-n3nccn3)n(C)c2n1]

In certain embodiments, the A2AR antagonist is an A2AR antagonist described in U.S. Pat. Nos. 8,114,845, 9,029,393, US20170015758, or US20160129108, herein incorporated by reference in their entirety.

In certain embodiments, the A2AR antagonist is istradefylline (CAS Registry Number: 155270-99-8). Istradefylline is also known as KW-6002 or 8-[(E)-2-(3,4-dimethoxyphenyl)vinyl]-1,3-diethyl-7-methyl-3,7-dihydro-1H-purine-2,6-dione. Istradefylline is disclosed, e.g., in LeWitt et al. (2008) Annals of Neurology 63 (3): 295-302).

In certain embodiments, the A2aR antagonist is tozadenant (Biotie). Tozadenant is also known as SYN115 or 4-hydroxy-N-(4-methoxy-7-morpholin-4-yl-1,3-benzothiazol-2-yl)-4-methylpiperidine-1-carboxamide. Tozadenant blocks the effect of endogenous adenosine at the A2a receptors, resulting in the potentiation of the effect of dopamine at the D2 receptor and inhibition of the effect of glutamate at the mGluR5 receptor. In some embodiments, the A2aR antagonist is preladenant (CAS Registry Number: 377727-87-2). Preladenant is also known as SCH 420814 or 2-(2-Furanyl)-7-[2-[4-[4-(2-methoxyethoxy)phenyl]-1-piperazinyl]ethyl]7H-pyrazolo[4,3-e][1,2,4]triazolo[1,5-c]pyrimidine-5-amine Preladenant was developed as a drug that acted as a potent and selective antagonist at the adenosine A2A receptor.

In certain embodiments, the A2aR antagonist is vipadenan. Vipadenan is also known as BIIB014, V2006, or 3-[(4-amino-3-methylphenyl)methyl]-7-(furan-2-yl)triazolo[4,5-d]pyrimidin-5-amine

Other exemplary A2aR antagonists include, e.g., ATL-444, MSX-3, SCH-58261, SCH-412,348, SCH-442,416, VER-6623, VER-6947, VER-7835, CGS-15943, or ZM-241,385.

In some embodiments, the A2aR antagonist is an A2aR pathway antagonist (e.g., a CD-73 inhibitor, e.g., an anti-CD73 antibody) is MEDI9447. MEDI9447 is a monoclonal antibody specific for CD73. Targeting the extracellular production of adenosine by CD73 may reduce the immunosuppressive effects of adenosine. MEDI9447 was reported to have a range of activities, e.g., inhibition of CD73 ectonucleotidase activity, relief from AMP-mediated lymphocyte suppression, and inhibition of syngeneic tumor growth. MEDI9447 can drive changes in both myeloid and lymphoid infiltrating leukocyte populations within the tumor microenvironment. These changes include, e.g., increases in CD8 effector cells and activated macrophages, as well as a reduction in the proportions of myeloid-derived suppressor cells (MDSC) and regulatory T lymphocytes.

### Exemplary PD-1 Inhibitors

In certain embodiments, the anti-CD73 antibody molecule described herein is administered in combination with a PD-1 inhibitor. The PD-1 inhibitor may be an antibody, an antigen binding fragment thereof, an immunoadhesin, a fusion protein, or an oligopeptide. In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune).

### Exemplary Anti-PD-1 Antibody Molecules

In one embodiment, the PD-1 inhibitor is an anti-PD-1 antibody molecule. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety.

In one embodiment, the anti-PD-1 antibody molecule comprises at least one, two, three, four, five or six complementarity determining regions (CDRs) (or collectively all of the CDRs) from a heavy and light chain variable region comprising an amino acid sequence shown in Table 5 (e.g., from the heavy and light chain variable region sequences of BAP049-Clone-E or BAP049-Clone-B disclosed in Table 5), or encoded by a nucleotide sequence shown in Table 5. In some embodiments, the CDRs are according to the Kabat definition (e.g., as set out in Table 5). In some embodiments, the CDRs are according to the Chothia definition (e.g., as set out in Table 5). In some embodiments, the CDRs are according to the combined CDR definitions of both Kabat and Chothia (e.g., as set out in Table 5). In one embodiment, the combination of Kabat and Chothia CDR of VH CDR1 comprises the amino acid sequence GYTFTTYWMH (SEQ ID NO: 541). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 5, or encoded by a nucleotide sequence shown in Table 5.

In one embodiment, the anti-PD-1 antibody molecule comprises a heavy chain variable region (VH) comprising a VHCDR1 amino acid sequence of SEQ ID NO: 501, a VHCDR2 amino acid sequence of SEQ ID NO: 502, and a VHCDR3 amino acid sequence of SEQ ID NO: 503; and a light chain variable region (VL) comprising a VLCDR1 amino acid sequence of SEQ ID NO: 510, a VLCDR2 amino acid sequence of SEQ ID NO: 511, and a VLCDR3 amino acid sequence of SEQ ID NO: 512, each disclosed in Table 5.

In one embodiment, the antibody molecule comprises a VH comprising a VHCDR1 encoded by the nucleotide sequence of SEQ ID NO: 524, a VHCDR2 encoded by the nucleotide sequence of SEQ ID NO: 525, and a VHCDR3 encoded by the nucleotide sequence of SEQ ID NO: 526; and a VL comprising a VLCDR1 encoded by the nucleotide sequence of SEQ ID NO: 529, a VLCDR2 encoded by the nucleotide sequence of SEQ ID NO: 530, and a VLCDR3 encoded by the nucleotide sequence of SEQ ID NO: 531, each disclosed in Table 5.

In one embodiment, the anti-PD-1 antibody molecule comprises a VH comprising the amino acid sequence of SEQ ID NO: 506, or an amino acid sequence having at least about 85%, 90%, 95%, or 99% sequence identity or higher to SEQ ID NO: 506. In one embodiment, the anti-PD-1 antibody molecule comprises a VL comprising the amino acid sequence of SEQ ID NO: 520, or an amino acid sequence having at least about 85%, 90%, 95%, or 99% sequence identity or higher to SEQ ID NO: 520. In one embodiment, the anti-PD-1 antibody molecule comprises a VL comprising the amino acid sequence of SEQ ID NO: 516, or an amino acid sequence having at least about 85%, 90%, 95%, or 99% sequence identity or higher to SEQ ID NO: 516. In one embodiment, the anti-PD-1 antibody molecule comprises a VH comprising the amino acid sequence of SEQ ID NO: 506 and a VL comprising the amino acid sequence of SEQ ID NO: 520. In one embodiment, the anti-PD-1 antibody molecule comprises a VH comprising the amino acid sequence of SEQ ID NO: 506 and a VL comprising the amino acid sequence of SEQ ID NO: 516.

In one embodiment, the antibody molecule comprises a VH encoded by the nucleotide sequence of SEQ ID NO: 507, or a nucleotide sequence having at least about 85%, 90%, 95%, or 99% sequence identity or higher to SEQ ID NO: 507. In one embodiment, the antibody molecule comprises a VL encoded by the nucleotide sequence of SEQ ID NO: 521 or 517, or a nucleotide sequence having at least about 85%, 90%, 95%, or 99% sequence identity or higher to SEQ ID NO: 521 or 517. In one embodiment, the antibody molecule comprises a VH encoded by the nucleotide sequence of SEQ ID NO: 507 and a VL encoded by the nucleotide sequence of SEQ ID NO: 521 or 517.

In one embodiment, the anti-PD-1 antibody molecule comprises a heavy chain comprising the amino acid sequence of SEQ ID NO: 508, or an amino acid sequence having at least about 85%, 90%, 95%, or 99% sequence identity or higher to SEQ ID NO: 508. In one embodiment, the anti-PD-1 antibody molecule comprises a light chain comprising the amino acid sequence of SEQ ID NO: 522, or an amino acid sequence having at least about 85%, 90%, 95%, or 99% sequence identity or higher to SEQ ID NO: 522. In one embodiment, the anti-PD-1 antibody molecule comprises a light chain comprising the amino acid sequence of SEQ ID NO: 518, or an amino acid sequence having at least about 85%, 90%, 95%, or 99% sequence identity or higher to SEQ ID NO: 518. In one embodiment, the anti-PD-1 antibody molecule comprises a heavy chain comprising the amino acid sequence of SEQ ID NO: 508 and a light chain comprising the amino acid sequence of SEQ ID NO: 522. In one embodiment, the anti-PD-1 antibody molecule comprises a heavy chain comprising the amino acid sequence of SEQ ID NO: 508 and a light chain comprising the amino acid sequence of SEQ ID NO: 518.

In one embodiment, the antibody molecule comprises a heavy chain encoded by the nucleotide sequence of SEQ ID NO: 509, or a nucleotide sequence having at least about 85%, 90%, 95%, or 99% sequence identity or higher to SEQ ID NO: 509. In one embodiment, the antibody molecule comprises a light chain encoded by the nucleotide sequence of SEQ ID NO: 523 or 519, or a nucleotide sequence having at least about 85%, 90%, 95%, or 99% sequence identity or higher to SEQ ID NO: 523 or 519. In one embodiment, the antibody molecule comprises a heavy chain encoded by the nucleotide sequence of SEQ ID NO: 509 and a light chain encoded by the nucleotide sequence of SEQ ID NO: 523 or 519.

The antibody molecules described herein can be made by vectors, host cells, and methods described in US 2015/0210769, incorporated by reference in its entirety.

TABLE 5
Amino acid and nucleotide sequences of exemplary anti-PD-1
antibody molecules

| | | |
|---|---|---|
| BAP049-Clone-B HC | | |
| SEQ ID NO: 501 | HCDR1 | TYWMH |
| (Rabat) | | |
| SEQ ID NO: 502 | HCDR2 | NIYPGTGGSNFDERFRN |
| (Rabat) | | |
| SEQ ID NO: 503 | HCDR3 | WTTGTGAY |
| (Rabat) | | |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVRRPGESLRISCRGSGYTFTTYWMHWVRQATGQG |
| | | LEWMGNIYPGTGGSNFDERFRNRVTITADRSTSTAYMELSSLRS |
| | | EDTAVYYCTRWTTGTGAYWGQGTTVTVSS |
| SEQ ID NO: 507 | DNA VH | GAGGTGCAGCTGGTGCAGTCAGGCGCCGAAGTGAAGAAGCCCG |
| | | GCGAGTCACTGAGAATTAGCTGTAAAGGTTCAGGCTACACCTT |
| | | CACTACCTACTGGATGCACTGGGTCCGCCAGGCTACCGGTCAA |
| | | GGCCTCGAGTGGATGGGTAATATCTACCCCGGCACCGGCGGCT |
| | | CTAACTTCGACGAGAAGTTTAAGAATAGAGTGACTATCACCGC |
| | | CGATAAGTCTACTAGCACCGCCTATATGGAACTGTCTAGCCTG |
| | | AGATCAGAGGACACCGCCGTCTACTACTGCACTAGGTGGACTA |
| | | CCGGCACAGGCGCCTACTGGGGTCAAGGCACTACCGTGACCGT |
| | | GTCTAGC |
| SEQ ID NO: 508 | Heavy | EVQLVQSGAEVRRPGESLRISCRGSGYTFTTYWMHWVRQATGQG |
| | chain | LEWMGNIYPGTGGSNFDERFRNRVTITADRSTSTAYMELSSLRSE |
| | | DTAVYYCTRWTTGTGAYWGQGTTVTVSSASTRGPSVFPLAPCSRS |
| | | TSESTAALGCLVRDYFPEPVTVSWNSGALTSGVHTFPAVLQSSGL |
| | | YSLSSVVTVPSSSLGTRTYTCNVDHRPSNTRVDRRVESRYGPPCP |
| | | PCPAPEFLGGPSVFLFPPRPRDTLMISRTPEVTCVVVDVSQEDPE |
| | | VQFNWYVDGVEVHNARTRPREEQFNSTYRVVSVLTVLHQDWLNGR |
| | | EYRCRVSNRGLPSSIERTISRARGQPREPQVYTLPPSQEEMTRNQ |
| | | VSLTCLVRGFYPSDIAVEWESNGQPENNYRTTPPVLDSDGSFFLY |
| | | SRLTVDRSRWQEGNVFSCSVMHEALHNHYTQRSLSLSLG |
| SEQ ID NO: 509 | DNA | GAGGTGCAGCTGGTGCAGTCAGGCGCCGAAGTGAAGAAGCCCG |
| | heavy | GCGAGTCACTGAGAATTAGCTGTAAAGGTTCAGGCTACACCTT |
| | chain | CACTACCTACTGGATGCACTGGGTCCGCCAGGCTACCGGTCAA |
| | | GGCCTCGAGTGGATGGGTAATATCTACCCCGGCACCGGCGGCT |
| | | CTAACTTCGACGAGAAGTTTAAGAATAGAGTGACTATCACCGC |
| | | CGATAAGTCTACTAGCACCGCCTATATGGAACTGTCTAGCCTGA |
| | | GATCAGAGGACACCGCCGTCTACTACTGCACTAGGTGGACTAC |
| | | CGGCACAGGCGCCTACTGGGGTCAAGGCACTACCGTGACCGTG |
| | | TCTAGCGCTAGCACTAAGGGCCCGTCCGTGTTCCCCCTGGCACC |
| | | TTGTAGCCGGAGCACTAGCGAATCCACCGCTGCCCTCGGCTGCC |
| | | TGGTCAAGGATTACTTCCCGGAGCCCGTGACCGTGTCCTGGAAC |
| | | AGCGGAGCCCTGACCTCCGGAGTGCACACCTTCCCCGCTGTGCT |
| | | GCAGAGCTCCGGGCTGTACTCGCTGTCGTCGGTGGTCACGGTGC |
| | | CTTCATCTAGCCTGGGTACCAAGACCTACACTTGCAACGTGGAC |
| | | CACAAGCCTTCCAACACTAAGGTGGACAAGCGCGTCGAATCGA |
| | | AGTACGGCCCACCGTGCCCGCCTTGTCCCGCGCCGGAGTTCCTC |
| | | GGCGGTCCCTCGGTCTTTCTGTTCCCACCGAAGCCCAAGGACAC |
| | | TTTGATGATTTCCCGCACCCCTGAAGTGACATGCGTGGTCGTGG |
| | | ACGTGTCACAGGAAGATCCGGAGGTGCAGTTCAATTGGTACGT |
| | | GGATGGCGTCGAGGTGCACAACGCCAAAACCAAGCCGAGGGA |
| | | GGAGCAGTTCAACTCCACTTACCGCGTCGTGTCCGTGCTGACGG |
| | | TGCTGCATCAGGACTGGCTGAACGGGAAGGAGTACAAGTGCAA |
| | | AGTGTCCAACAAGGGACTTCCTAGCTCAATCGAAAAGACCATC |
| | | TCGAAAGCCAAGGGACAGCCCCGGGAACCCCAAGTGTATACCC |
| | | TGCCACCGAGCCAGGAAGAAATGACTAAGAACCAAGTCTCATT |
| | | GACTTGCCTTGTGAAGGGCTTCTACCCATCGGATATCGCCGTGG |
| | | AATGGGAGTCCAACGGCCAGCCGGAAAACAACTACAAGACCA |
| | | CCCCTCCGGTGCTCGACTCAGACGGATCCTTCTTCCTCTACTCG |
| | | CGGCTGACCGTGGATAAGAGCAGATGGCAGGAGGGAAATGTGT |
| | | TCAGCTGTTCTGTGATGCATGAAGCCCTGCACAACCACTACACT |
| | | CAGAAGTCCCTGTCCCTCTCCCTGGGA |
| BAP049-Clone-B LC | | |
| SEQ ID NO: | LCDR1 | KSSQSLLDSGNQKNFLT |
| 510 (Kabat) | | |
| SEQ ID NO: | LCDR2 | WASTRES |
| 511 (Kabat) | | |
| SEQ ID NO: | LCDR3 | QNDYSYPYT |
| 512 (Kabat) | | |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 516 | VL | EIVLTQSPATLSLSPGERATLSCKSSQSLLDSGNQKNELTWYQQKP |
| | | GKAPKLLIYWASTRESGVPSRFSGSGSGTDFTFTISSLQPEDIATYY |
| | | CQNDYSYPYTFGQGTKVEIK |
| SEQ ID NO: 517 | DNA VL | GAGATCGTCCTGACTCAGTCACCCGCTACCCTGAGCCTGAGCCC |
| | | TGGCGAGCGGGCTACACTGAGCTGTAAATCTAGTCAGTCACTG |
| | | CTGGATAGCGGTAATCAGAAGAACTTCCTGACCTGGTATCAGC |
| | | AGAAGCCCGGTAAAGCCCCTAAGCTGCTGATCTACTGGGCCTC |
| | | TACTAGAGAATCAGGCGTGCCCTCTAGGTTTAGCGGTAGCGGT |
| | | AGTGGCACCGACTTCACCTTCACTATCTCTAGCCTGCAGCCCGA |
| | | GGATATCGCTACCTACTACTGTCAGAACGACTATAGCTACCCCT |
| | | ACACCTTCGGTCAAGGCACTAAGGTCGAGATTAAG |
| SEQ ID NO: 518 | Light | EIVLTQSPATLSLSPGERATLSCRSSQSLLDSGNQRNFLTWYQQRP |
| | chain | GRAPRLLIYWASTRESGVPSRFSGSGSGTDFTFTISSLQPEDIATYY |
| | | CQNDYSYPYTFGQGTKVEIKRTVAAPSVFIFPPSDEQLKSGTASVV |
| | | CLLNNFYPREARVQWRVDNALQSGNSQESVTEQDSRDSTYSLSST |
| | | LTLSRADYERHRVYACEVTHQGLSSPVTRSFNRGEC |
| SEQ ID NO: 519 | DNA | GAGATCGTCCTGACTCAGTCACCCGCTACCCTGAGCCTGAGCCC |
| | light | TGGCGAGCGGGCTACACTGAGCTGTAAATCTAGTCAGTCACTG |
| | chain | CTGGATAGCGGTAATCAGAAGAACTTCCTGACCTGGTATCAGC |
| | | AGAAGCCCGGTAAAGCCCCTAAGCTGCTGATCTACTGGGCCTC |
| | | TACTAGAGAATCAGGCGTGCCCTCTAGGTTTAGCGGTAGCGGT |
| | | AGTGGCACCGACTTCACCTTCACTATCTCTAGCCTGCAGCCCGA |
| | | GGATATCGCTACCTACTACTGTCAGAACGACTATAGCTACCCCT |
| | | ACACCTTCGGTCAAGGCACTAAGGTCGAGATTAAGCGTACGGT |
| | | GGCCGCTCCCAGCGTGTTCATCTTCCCCCCCAGCGACGAGCAGC |
| | | TGAAGAGCGGCACCGCCAGCGTGGTGTGCCTGCTGAACAACTT |
| | | CTACCCCCGGGAGGCCAAGGTGCAGTGGAAGGTGGACAACGCC |
| | | CTGCAGAGCGGCAACAGCCAGGAGAGCGTCACCGAGCAGGAC |
| | | AGCAAGGACTCCACCTACAGCCTGAGCAGCACCCTGACCCTGA |
| | | GCAAGGCCGACTACGAGAAGCATAAGGTGTACGCCTGCGAGGT |
| | | GACCCACCAGGGCCTGTCCAGCCCCGTGACCAAGAGCTTCAAC |
| | | AGGGGCGAGTGC |
| BAP049-Clone-E HC | | |
| SEQ ID NO: 501 | HCDR1 | TYWMH |
| (Rabat) | | |
| SEQ ID NO: 502 | HCDR2 | NIYPGTGGSNFDEKFKN |
| (Rabat) | | |
| SEQ ID NO: 503 | HCDR3 | WTTGTGAY |
| (Rabat) | | |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVRRPGESLRISCKGSGYTFTTYWMHWVRQATGQG |
| | | LEWMGNIYPGTGGSNFDERFRNRVTITADRSTSTAYMELSSLRS |
| | | EDTAVYYCTRWTTGTGAYWGQGTTVTVSS |
| SEQ ID NO: 507 | | GAGGTGCAGCTGGTGCAGTCAGGCGCCGAAGTGAAGAAGCCCG |
| | | GCGAGTCACTGAGAATTAGCTGTAAAGGTTCAGGCTACACCTT |
| | | CACTACCTACTGGATGCACTGGGTCCGCCAGGCTACCGGTCAA |
| | | GGCCTCGAGTGGATGGGTAATATCTACCCCGGCACCGGCGGCT |
| | | CTAACTTCGACGAGAAGTTTAAGAATAGAGTGACTATCACCGC |
| | | CGATAAGTCTACTAGCACCGCCTATATGGAACTGTCTAGCCTG |
| | | AGATCAGAGGACACCGCCGTCTACTACTGCACTAGGTGGACTA |
| | | CCGGCACAGGCGCCTACTGGGGTCAAGGCACTACCGTGACCGT |
| | | G |
| SEQ ID NO: 508 | Heavy | EVQLVQSGAEVKKPGESLR1SCKGSGYTFTTYWMFIWVRQATGQG |
| | chain | LEWMGNIYPGTGGSNFDEKFKNRVTITADKSTSTAYMELSSLRSE |
| | | DTAVYYCTRWTTGTGAYWGQGTTVTVSSASTKGPSVFPLAPCSRS |
| | | TSESTAALGCLVKDYFPEPVTVSWNSGALTSGVHTFPAVLQSSGL |
| | | YSLSSVVTVPSSSLGTKTYTCNVDHKPSNTKVDKRVESKYGPPCPP |
| | | CPAPEFLGGPSVFLFPPKPKDTLMISRTPEVTCVVVDVSQEDPEVQ |
| | | FNWYVDGVEVHNAKTKPREEQFNSTYRVVSVLTVLHQDWLNGKEYK |
| | | CKVSNKGLPSSIEKTISKAKGQPREPQVYTLPPSQEEMTKNQVSLT |
| | | CLVKGFYPSDIAVEWESNGQPENNYKTTPPVLDSDGSFFLYSRLTV |
| | | DKSRWQEGNVFSCSVMHEALHNHYTQKSLSLSLG |
| SEQ ID NO: 509 | DNA | GAGGTGCAGCTGGTGCAGTCAGGCGCCGAAGTGAAGAAGCCCGGCG |
| | heavy | AGTCACTGAGAATTAGCTGTAAAGGTTCAGGCTACACCTTCACTAC |
| | chain | CTACTGGATGCACTGGGTCCGCCAGGCTACCGGTCAAGGCCTCGAG |
| | | TGGATGGGTAATATCTACCCCGGCACCGGCGGCTCTAACTTCGACG |
| | | AGAAGTTTAAGAATAGAGTGACTATCACCGCCGATAAGTCTACTAG |
| | | CACCGCCTATATGGAACTGTCTAGCCTGAGATCAGAGGACACCGCC |
| | | GTCTACTACTGCACTAGGTGGACTACCGGCACAGGCGCCTACTGGG |
| | | GTCAAGGCACTACCGTGACCGTGTCTAGCGCTAGCACTAAGGGCCC |
| | | GTCCGTGTTCCCCCTGGCACCTTGTAGCCGGAGCACTAGCGAATCC |
| | | ACCGCTGCCCTCGGCTGCCTGGTCAAGGATTACTTCCCGGAGCCCG |
| | | TGACCGTGTCCTGGAACAGCGGAGCCCTGACCTCCGGAGTGCACAC |
| | | CTTCCCCGCTGTGCTGCAGAGCTCCGGGCTGTACTCGCTGTCGTCG |
| | | GTGGTCACGGTGCCTTCATCTAGCCTGGGTACCAAGACCTACACTT |
| | | GCAACGTGGACCACAAGCCTTCCAACACTAAGGTGGACAAGCGCGT |
| | | CGAATCGAAGTACGGCCCACCGTGCCCGCCTTGTCCCGCGCCGGAG |
| | | TTCCTCGGCGGTCCCTCGGTCTTTCTGTTCCCACCGAAGCCCAAGG |
| | | ACACTTTGATGATTTCCCGCACCCCTGAAGTGACATGCGTGGTCGT |
| | | GGACGTGTCACAGGAAGATCCGGAGGTGCAGTTCAATTGGTACGTG |
| | | GATGGCGTCGAGGTGCACAACGCCAAAACCAAGCCGAGGGAGGAGC |
| | | AGTTCAACTCCACTTACCGCGTCGTGTCCGTGCTGACGGTGCTGCA |
| | | TCAGGACTGGCTGAACGGGAAGGAGTACAAGTGCAAAGTGTCCAAC |
| | | AAGGGACTTCCTAGCTCAATCGAAAAGACCATCTCGAAAGCCAAGG |
| | | GACAGCCCCGGGAACCCCAAGTGTATACCCTGCCACCGAGCCAGGA |
| | | AGAAATGACTAAGAACCAAGTCTCATTGACTTGCCTTGTGAAGGGC |
| | | TTCTACCCATCGGATATCGCCGTGGAATGGGAGTCCAACGGCCAGC |
| | | CGGAAAACAACTACAAGACCACCCCTCCGGTGCTGGACTCAGACGG |
| | | ATCCTTCTTCCTCTACTCGCGGCTGACCGTGGATAAGAGCAGATGG |
| | | CAGGAGGGAAATGTGTTCAGCTGTTCTGTGATGCATGAAGCCCTGC |
| | | ACAACCACTACACTCAGAAGTCCCTGTCCCTCTCCCTGGGA |
| BAP049-Clone-E LC | | |
| SEQ ID NO: 510 | LCDR1 | KSSQSLLDSGNQKNFLT |
| (Kabat) | | |
| SEQ ID NO: 511 | LCDR2 | WASTRES |
| (Kabat) | | |
| SEQ ID NO: 512 | LCDR3 | QNDYSYPYT |
| (Kabat) | | |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 520 | VL | EIVLTQSPATLSLSPGERATLSCKSSQSLLDSGNQKNFLTWYQQKP |
| | | GQAPRLLIYWASTRESGVPSRFSGSGSGTDFTFTISSLEAEDAATYY |
| | | CQNDYSYPYTFGQGTKVEIK |
| SEQ ID NO: 521 | DNA VL | GAGATCGTCCTGACTCAGTCACCCGCTACCCTGAGCCTGAGCCC |
| | | TGGCGAGCGGGCTACACTGAGCTGTAAATCTAGTCAGTCACTG |
| | | CTGGATAGCGGTAATCAGAAGAACTTCCTGACCTGGTATCAGC |
| | | AGAAGCCCGGTCAAGCCCCTAGACTGCTGATCTACTGGGCCTCT |
| | | ACTAGAGAATCAGGCGTGCCCTCTAGGTTTAGCGGTAGCGGTA |
| | | GTGGCACCGACTTCACCTTCACTATCTCTAGCCTGGAAGCCGAG |
| | | GACGCCGCTACCTACTACTGTCAGAACGACTATAGCTACCCCTA |
| | | CACCTTCGGTCAAGGCACTAAGGTCGAGATTAAG |
| SEQ ID NO: 522 | Light | EIVLTQSPATLSLSPGERATLSCKSSQSLLDSGNQKNFLTWYQQKP |
| | chain | GQAPRLLIYWASTRESGVPSRFSGSGSGTDFTFTISSLEAEDAATYY |
| | | CQNDYSYPYTFGQGTKVEIKRTVAAPSVFIFPPSDEQLKSGTASVV |
| | | CLLNNFYPREAKVQWKVDNALQSGNSQESVTEQDSKDSTYSLSST |
| | | LTLSKADYEKHKVYACEVTHQGLSSPVTKSFNRGEC |
| SEQ ID NO: 523 | DNA | GAGATCGTCCTGACTCAGTCACCCGCTACCCTGAGCCTGAGCCC |
| | light | TGGCGAGCGGGCTACACTGAGCTGTAAATCTAGTCAGTCACTG |
| | chain | CTGGATAGCGGTAATCAGAAGAACTTCCTGACCTGGTATCAGC |
| | | AGAAGCCCGGTCAAGCCCCTAGACTGCTGATCTACTGGGCCTCT |
| | | ACTAGAGAATCAGGCGTGCCCTCTAGGTTTAGCGGTAGCGGTA |
| | | GTGGCACCGACTTCACCTTCACTATCTCTAGCCTGGAAGCCGAG |
| | | GACGCCGCTACCTACTACTGTCAGAACGACTATAGCTACCCCTA |
| | | CACCTTCGGTCAAGGCACTAAGGTCGAGATTAAGCGTACGGTG |
| | | GCCGCTCCCAGCGTGTTCATCTTCCCCCCCAGCGACGAGCAGCT |
| | | GAAGAGCGGCACCGCCAGCGTGGTGTGCCTGCTGAACAACTTC |
| | | TACCCCCGGGAGGCCAAGGTGCAGTGGAAGGTGGACAACGCCC |
| | | TGCAGAGCGGCAACAGCCAGGAGAGCGTCACCGAGCAGGACA |
| | | GCAAGGACTCCACCTACAGCCTGAGCAGCACCCTGACCCTGAG |
| | | CAAGGCCGACTACGAGAAGCATAAGGTGTACGCCTGCGAGGTG |
| | | ACCCACCAGGGCCTGTCCAGCCCCGTGACCAAGAGCTTCAACA |
| | | GGGGCGAGTGC |
| BAP049-Clone-B HC | | |
| SEQ ID NO: 524 | HCDR1 | ACCTACTGGATGCAC |
| (Kabat) | | |
| SEQ ID NO: 525 | HCDR2 | AATATCTACCCCGGCACCGGCGGCTCTAACTTCGACGAGAAGT |
| (Kabat) | | TTAAGAAT |
| SEQ ID NO: 526 | HCDR3 | TGGACTACCGGCACAGGCGCCTAC |
| (Kabat) | | |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | |
| (Chothia) | | TGGACTACCGGCACAGGCGCCTAC |
| BAP049-Clone-B LC | | |
| SEQ ID NO: 529 | LCDR1 | AAATCTAGTCAGTCACTGCTGGATAGCGGTAATCAGAAGAACT |
| (Kabat) | | TCCTGACC |
| SEQ ID NO: 530 | LCDR2 | TGGGCCTCTACTAGAGAATCA |
| (Kabat) | | |
| SEQ ID NO: 531 | LCDR3 | CAGAACGACTATAGCTACCCCTACACC |
| (Kabat) | | |
| SEQ ID NO: 532 | LCDR1 | AGTCAGTCACTGCTGGATAGCGGTAATCAGAAGAACTTC |
| (Chothia) | | |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |
| BAP049-Clone-E HC | | |
| SEQ ID NO: 524 | HCDR1 | ACCTACTGGATGCAC |
| (Kabat) | | |
| SEQ ID NO: 525 | HCDR2 | AATATCTACCCCGGCACCGGCGGCTCTAACTTCGACG |
| (Kabat) | | AGAAGTTTAAGAAT |
| SEQ ID NO: 526 | HCDR3 | TGGACTACCGGCACAGGCGCCTAC |
| (Kabat) | | |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |
| BAP049-Clone-E LC | | |
| SEQ ID NO: 529 | LCDR1 | AAATCTAGTCAGTCACTGCTGGATAGCGGTAATCAGAA |
| (Kabat) | | GAACTTCCTGACC |
| SEQ ID NO: 530 | LCDR2 | TGGGCCTCTACTAGAGAATCA |
| (Kabat) | | |
| SEQ ID NO: 531 | LCDR3 | CAGAACGACTATAGCTACCCCTACACC |
| (Kabat) | | |
| SEQ ID NO: 532 | LCDR1 | AGTCAGTCACTGCTGGATAGCGGTAATCAGAAGAACTTC |
| (Chothia) | | |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |

### Other Exemplary PD-1 Inhibitors

In one embodiment, the anti-PD-1 antibody molecule is Nivolumab (Bristol-Myers Squibb), also known as MDX-1106, MDX-1106-04, ONO-4538, BMS-936558, or OPDIVO®. Nivolumab (clone 5C4) and other anti-PD-1 antibodies are disclosed in U.S. Pat. No. 8,008,449 and WO 2006/121168, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Nivolumab, e.g., as disclosed in Table 6.

In one embodiment, the anti-PD-1 antibody molecule is Pembrolizumab (Merck & Co), also known as Lambrolizumab, MK-3475, MK03475, SCH-900475, or KEYTRUDA®. Pembrolizumab and other anti-PD-1 antibodies are disclosed in Hamid, O. et al. (2013) New England Journal of Medicine 369 (2): 134-44, U.S. Pat. No. 8,354,509, and WO 2009/114335, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pembrolizumab, e.g., as disclosed in Table 6.

In one embodiment, the anti-PD-1 antibody molecule is Pidilizumab (CureTech), also known as CT-011. Pidilizumab and other anti-PD-1 antibodies are disclosed in Rosenblatt, J. et al. (2011) J Immunotherapy 34(5): 409-18, U.S. Pat. Nos. 7,695,715, 7,332,582, and 8,686,119, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pidilizumab, e.g., as disclosed in Table 6.

In one embodiment, the anti-PD-1 antibody molecule is MEDI0680 (Medimmune), also known as AMP-514. MEDI0680 and other anti-PD-1 antibodies are disclosed in U.S. Pat. No. 9,205,148 and WO 2012/145493, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of MEDI0680.

In one embodiment, the anti-PD-1 antibody molecule is REGN2810 (Regeneron). In one embodiment, the anti-PD-1 antibody molecule comprises one or more of the CDR sequences (or collectively all of the CDR sequences), the heavy chain or light chain variable region sequence, or the heavy chain or light chain sequence of REGN2810.

In one embodiment, the anti-PD-1 antibody molecule is PF-06801591 (Pfizer). In one embodiment, the anti-PD-1 antibody molecule comprises one or more of the CDR sequences (or collectively all of the CDR sequences), the heavy chain or light chain variable region sequence, or the heavy chain or light chain sequence of PF-06801591.

In one embodiment, the anti-PD-1 antibody molecule is BGB-A317 or BGB-108 (Beigene). In one embodiment, the anti-PD-1 antibody molecule comprises one or more of the CDR sequences (or collectively all of the CDR sequences), the heavy chain or light chain variable region sequence, or the heavy chain or light chain sequence of BGB-A317 or BGB-108.

In one embodiment, the anti-PD-1 antibody molecule is INCSHR1210 (Incyte), also known as INCSHR01210 or SHR-1210. In one embodiment, the anti-PD-1 antibody molecule comprises one or more of the CDR sequences (or collectively all of the CDR sequences), the heavy chain or light chain variable region sequence, or the heavy chain or light chain sequence of INCSHR1210.

In one embodiment, the anti-PD-1 antibody molecule is TSR-042 (Tesaro), also known as ANB011. In one embodiment, the anti-PD-1 antibody molecule comprises one or more of the CDR sequences (or collectively all of the CDR sequences), the heavy chain or light chain variable region sequence, or the heavy chain or light chain sequence of TSR-042.

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated by reference in their entirety).

TABLE 6
Amino acid sequences of other exemplary
anti-PD-1 antibody molecules

| | | |
|---|---|---|
| Nivolumab | | |
| SEQ ID NO: 535 | Heavy | QVQLVESGGGVVQPGRSLRLDCKA |
| | chain | SGITFSNSGMHWVRQAPGKGLEWV |
| | | AVIWYDGSKRYYADSVKGRFTISR |
| | | DNSKNTLFLQMNSLRAEDTAVYYC |
| | | ATNDDYWGQGTLVTVSSASTKGPS |
| | | VFPLAPCSRSTSESTAALGCLVKD |
| | | YFPEPVTVSWNSGALTSGVHTFPA |
| | | VLQSSGLYSLSSVVTVPSSSLGTK |
| | | TYTCNVDHKPSNTKVDKRVESKYG |
| | | PPCPPCPAPEFLGGPSVFLFPPKP |
| | | KDTLMISRTPEVTCVVVDVSQEDP |
| | | EVQFNWYVDGVEVHNAKTKPREEQ |
| | | FNSTYRVVSVLTVLHQDWLNGKEY |
| | | KCKVSNKGLPSSIEKTISKAKGQP |
| | | REPQVYTLPPSQEEMTKNQVSLTC |
| | | LVKGFYPSDIAVEWESNGQPENNY |
| | | KTTPPVLDSDGSFFLYSRLTVDKS |
| | | RWQEGNVFSCSVMHEALHNHYTQK |
| | | SLSLSLGK |
| SEQ ID NO: 536 | Light | EIVLTQSPATLSLSPGERATLSCR |
| | chain | ASQSVSSYLAWYQQKPGQAPRLLI |
| | | YDASNRATGIPARFSGSGSGTDFT |
| | | LTISSLEPEDFAVYYCQQSSNWPR |
| | | TFGQGTKVEIKRTVAAPSVFIFPP |
| | | SDEQLKSGTASVVCLLNNFYPREA |
| | | KVQWKVDNALQSGNSQESVTEQDS |
| | | KDSTYSLSSTLTLSKADYEKHKVY |
| | | ACEVTHQGLSSPVTKSFNRGEC |
| Pembrolizumab | | |
| SEQ ID NO: 537 | Heavy | QVQLVQSGVEVKKPGASVKVSCKAS |
| | chain | GYTFTNYYMYWVRQAPGQGLEWMGG |
| | | INPSNGGTNFNEKFKNRVTLTTDSS |
| | | TTTAYMELKSLQFDDTAVYYCARRD |
| | | YRFDMGFDYWGQGTTVTVSSASTKG |
| | | PSVFPLAPCSRSTSESTAALGCLVK |
| | | DYFPEPVTVSWNSGALTSGVHTFPA |
| | | VLQSSGLYSLSSVVTVPSSSLGTKT |
| | | YTCNVDHKPSNTKVDKRVESKYGPP |
| | | CPPCPAPEFLGGPSVFLFPPKPKDT |
| | | LMISRTPEVTCVVVDVSQEDPEVQF |
| | | NWYVDGVEVHNAKTKPREEQFNSTY |
| | | RVVSVLTVLHQDWLNGKEYKCKVSN |
| | | KGLPSSIEKTISKAKGQPREPQVYT |
| | | LPPSQEEMTKNQVSLTCLVKGFYPS |
| | | DIAVEWESNGQPENNYKTTPPVLDS |
| | | DGSFFLYSRLTVDKSRWQEGNVFSC |
| | | SVMHEALHNHYTQKSLSLSLGK |
| SEQ ID NO: 538 | Light | EIVLTQSPATLSLSPGERATLS |
| | chain | CRASKGVSTSGYSYLHWYQQKP |
| | | GQAPRLLIYLASYLESGVPARF |
| | | SGSGSGTDFTLTISSLEPEDFA |
| | | VYYCQHSRDLPLTFGGGTKVEI |
| | | KRTVAAPSVFIFPPSDEQLKSG |
| | | TASVVCLLNNFYPREAKVQWKV |
| | | DNALQSGNSQESVTEQDSKDST |
| | | YSLSSTLTLSKADYEKHKVYAC |
| | | EVTHQGLSSPVTKSFNRGEC |
| Pidilizumab | | |
| SEQ ID NO: 539 | Heavy | QVQLVQSGSELKKPGASVKISC |
| | chain | KASGYTFTNYGMNWVRQAPGQG |
| | | LQWMGWINTDSGESTYAEEFKG |
| | | RFVFSLDTSVNTAYLQITSLTA |
| | | EDTGMYFCVRVGYDALDYWGQG |
| | | TLVTVSSASTKGPSVFPLAPSS |
| | | KSTSGGTAALGCLVKDYFPEPV |
| | | TVSWNSGALTSGVHTFPAVLQS |
| | | SGLYSLSSVVTVPSSSLGTQTY |
| | | ICNVNHKPSNTKVDKRVEPKSC |
| | | DKTHTCPPCPAPELLGGPSVFL |
| | | FPPKPKDTLMISRTPEVTCVVV |
| | | DVSHEDPEVKFNWYVDGVEVHN |
| | | AKTKPREEQYNSTYRVVSVLTV |
| | | LHQDWLNGKEYKCKVSNKALPA |
| | | PIEKTISKAKGQPREPQVYTLP |
| | | PSREEMTKNQVSLTCLVKGFYP |
| | | SDIAVEWESNGQPENNYKTTPP |
| | | VLDSDGSFFLYSKLTVDKSRWQ |
| | | QGNVFSCSVMHEALHNHYTQKS |
| | | LSLSPGK |
| SEQ ID NO: 540 | Light | EIVLTQSPSSLSASVGDRVTIT |
| | chain | CSARSSVSYMHWFQQKPGKAPK |
| | | LWIYRTSNLASGVPSRFSGSGS |
| | | GTSYCLTINSLQPEDFATYYCQ |
| | | QRSSFPLTFGGGTKLEIKRTVA |
| | | APSVFTFPPSDEQLKSGTASVV |
| | | CLLNNFYPREAKVQWKVDNALQ |
| | | SGNSQESVTEQDSKDSTYSLSS |
| | | TLTLSKADYEKHKVYACEVTHQ |
| | | GLSSPVTKSFNRGEC |

### Exemplary PD-L1 Inhibitors

In certain embodiments, the anti-CD73 antibody molecule described herein is administered in combination with a PD-L1 inhibitor. The PD-L1 inhibitor may be an antibody, an antigen binding fragment thereof, an immunoadhesin, a fusion protein, or an oligopeptide. In some embodiments, the PD-L1 inhibitor is chosen from FAZ053 (Novartis), Atezolizumab (Genentech/Roche), Avelumab (Merck Serono and Pfizer), Durvalumab (Medlmmune/AstraZeneca), or BMS-936559 (Bristol-Myers Squibb).

### Exemplary Anti-PD-L1 Antibody Molecules

In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety.

In one embodiment, the anti-PD-L1 antibody molecule comprises at least one, two, three, four, five or six complementarity determining regions (CDRs) (or collectively all of the CDRs) from a heavy and light chain variable region comprising an amino acid sequence shown in Table 7 (e.g., from the heavy and light chain variable region sequences of BAP058-Clone 0 or BAP058-Clone N disclosed in Table 7), or encoded by a nucleotide sequence shown in Table 7. In some embodiments, the CDRs are according to the Kabat definition (e.g., as set out in Table 7). In some embodiments, the CDRs are according to the Chothia definition (e.g., as set out in Table 7). In some embodiments, the CDRs are according to the combined CDR definitions of both Kabat and Chothia (e.g., as set out in Table 7). In one embodiment, the combination of Kabat and Chothia CDR of VH CDR1 comprises the amino acid sequence GYTFTSYWMY (SEQ ID NO: 647). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 7, or encoded by a nucleotide sequence shown in Table 7.

In one embodiment, the anti-PD-L1 antibody molecule comprises a heavy chain variable region (VH) comprising a VHCDR1 amino acid sequence of SEQ ID NO: 601, a VHCDR2 amino acid sequence of SEQ ID NO: 602, and a VHCDR3 amino acid sequence of SEQ ID NO: 603; and a light chain variable region (VL) comprising a VLCDR1 amino acid sequence of SEQ ID NO: 609, a VLCDR2 amino acid sequence of SEQ ID NO: 610, and a VLCDR3 amino acid sequence of SEQ ID NO: 611, each disclosed in Table 7.

In one embodiment, the anti-PD-L1 antibody molecule comprises a VH comprising a VHCDR1 encoded by the nucleotide sequence of SEQ ID NO: 628, a VHCDR2 encoded by the nucleotide sequence of SEQ ID NO: 629, and a VHCDR3 encoded by the nucleotide sequence of SEQ ID NO: 630; and a VL comprising a VLCDR1 encoded by the nucleotide sequence of SEQ ID NO: 633, a VLCDR2 encoded by the nucleotide sequence of SEQ ID NO: 634, and a VLCDR3 encoded by the nucleotide sequence of SEQ ID NO: 635, each disclosed in Table 7.

In one embodiment, the anti-PD-L1 antibody molecule comprises a VH comprising the amino acid sequence of SEQ ID NO: 606, or an amino acid sequence having at least about 85%, 90%, 95%, or 99% sequence identity to SEQ ID NO: 606. In one embodiment, the anti-PD-L1 antibody molecule comprises a VL comprising the amino acid sequence of SEQ ID NO: 616, or an amino acid sequence having at least about 85%, 90%, 95%, or 99% sequence identity or higher to SEQ ID NO: 616. In one embodiment, the anti-PD-L1 antibody molecule comprises a VH comprising the amino acid sequence of SEQ ID NO: 620, or an amino acid sequence having at least about 85%, 90%, 95%, or 99% sequence identity to SEQ ID NO: 620. In one embodiment, the anti-PD-L1 antibody molecule comprises a VL comprising the amino acid sequence of SEQ ID NO: 624, or an amino acid sequence having at least about 85%, 90%, 95%, or 99% sequence identity to SEQ ID NO: 624. In one embodiment, the anti-PD-L1 antibody molecule comprises a VH comprising the amino acid sequence of SEQ ID NO: 606 and a VL comprising the amino acid sequence of SEQ ID NO: 616. In one embodiment, the anti-PD-L1 antibody molecule comprises a VH comprising the amino acid sequence of SEQ ID NO: 620 and a VL comprising the amino acid sequence of SEQ ID NO: 624.

In one embodiment, the antibody molecule comprises a VH encoded by the nucleotide sequence of SEQ ID NO: 607, or a nucleotide sequence having at least about 85%, 90%, 95%, or 99% sequence identity to SEQ ID NO: 607. In one embodiment, the antibody molecule comprises a VL encoded by the nucleotide sequence of SEQ ID NO: 617, or a nucleotide sequence having at least about 85%, 90%, 95%, or 99% sequence identity to SEQ ID NO: 617. In one embodiment, the antibody molecule comprises a VH encoded by the nucleotide sequence of SEQ ID NO: 621, or a nucleotide sequence having at least about 85%, 90%, 95%, or 99% sequence identity to SEQ ID NO: 621. In one embodiment, the antibody molecule comprises a VL encoded by the nucleotide sequence of SEQ ID NO: 625, or a nucleotide sequence having at least about 85%, 90%, 95%, or 99% sequence identity to SEQ ID NO: 625. In one embodiment, the antibody molecule comprises a VH encoded by the nucleotide sequence of SEQ ID NO: 607 and a VL encoded by the nucleotide sequence of SEQ ID NO: 617. In one embodiment, the antibody molecule comprises a VH encoded by the nucleotide sequence of SEQ ID NO: 621 and a VL encoded by the nucleotide sequence of SEQ ID NO: 625.

In one embodiment, the anti-PD-L1 antibody molecule comprises a heavy chain comprising the amino acid sequence of SEQ ID NO: 608, or an amino acid sequence having at least about 85%, 90%, 95%, or 99% sequence identity to SEQ ID NO: 608. In one embodiment, the anti-PD-L1 antibody molecule comprises a light chain comprising the amino acid sequence of SEQ ID NO: 618, or an amino acid sequence having at least about 85%, 90%, 95%, or 99% sequence identity to SEQ ID NO: 618. In one embodiment, the anti-PD-L1 antibody molecule comprises a heavy chain comprising the amino acid sequence of SEQ ID NO: 622, or an amino acid sequence having at least about 85%, 90%, 95%, or 99% sequence identity to SEQ ID NO: 622. In one embodiment, the anti-PD-L1 antibody molecule comprises a light chain comprising the amino acid sequence of SEQ ID NO: 626, or an amino acid sequence having at least about 85%, 90%, 95%, or 99% sequence identity to SEQ ID NO: 626. In one embodiment, the anti-PD-L1 antibody molecule comprises a heavy chain comprising the amino acid sequence of SEQ ID NO: 608 and a light chain comprising the amino acid sequence of SEQ ID NO: 618. In one embodiment, the anti-PD-L1 antibody molecule comprises a heavy chain comprising the amino acid sequence of SEQ ID NO: 622 and a light chain comprising the amino acid sequence of SEQ ID NO: 626.

In one embodiment, the antibody molecule comprises a heavy chain encoded by the nucleotide sequence of SEQ ID NO: 615, or a nucleotide sequence having at least about 85%, 90%, 95%, or 99% sequence identity to SEQ ID NO: 615. In one embodiment, the antibody molecule comprises a light chain encoded by the nucleotide sequence of SEQ ID NO: 619, or a nucleotide sequence having at least about 85%, 90%, 95%, or 99% sequence identity to SEQ ID NO: 619. In one embodiment, the antibody molecule comprises a heavy chain encoded by the nucleotide sequence of SEQ ID NO: 623, or a nucleotide sequence having at least about 85%, 90%, 95%, or 99% sequence identity to SEQ ID NO: 623. In one embodiment, the antibody molecule comprises a light chain encoded by the nucleotide sequence of SEQ ID NO: 627, or a nucleotide sequence having at least about 85%, 90%, 95%, or 99% sequence identity to SEQ ID NO: 627. In one embodiment, the antibody molecule comprises a heavy chain encoded by the nucleotide sequence of SEQ ID NO: 615 and a light chain encoded by the nucleotide sequence of SEQ ID NO: 619. In one embodiment, the antibody molecule comprises a heavy chain encoded by the nucleotide sequence of SEQ ID NO: 623 and a light chain encoded by the nucleotide sequence of SEQ ID NO: 627.

The antibody molecules described herein can be made by vectors, host cells, and methods described in US 2016/0108123, incorporated by reference in its entirety.

TABLE 7
Amino acid and nucleotide sequences of exemplary
anti-PD-L1 antibody molecules

| | | |
|---|---|---|
| BAP058-Clone O HC | | |
| SEQ ID NO: 601 (Kabat) | HCDR1 | SYWMY |
| SEQ ID NO: 602 (Kabat) | HCDR2 | RIDPNSGSTKYNEKFKN |
| SEQ ID NO: 603 (Kabat) | HCDR3 | DYRKGLYAMDY |
| SEQ ID NO: 604 | HCDR1 | GYTFTSY |
| (Chothia) | | |
| SEQ ID NO: 605 | HCDR2 | DPNSGS |
| (Chothia) | | |
| SEQ ID NO: 603 | HCDR3 | DYRKGLYAMDY |
| (Chothia) | | |
| SEQ ID NO: 606 | VH | EVQLVQSGAEVKKPGATVKISCKVSGYTFTSYWMYWVRQARGQ |
| | | RLEWIGRIDPNSGSTKYNEKFKNRFTISRDNSKNTLYLQMNSLRA |
| | | EDTAVYYCARDYRKGLYAMDYWGQGTTVTVSS |
| SEQ ID NO: 607 | DNA VH | GAAGTGCAGCTGGTGCAGTCAGGCGCCGAAGTGAAGAAACCC |
| | | GGCGCTACCGTGAAGATTAGCTGTAAAGTCTCAGGCTACACCT |
| | | TCACTAGCTACTGGATGTACTGGGTCCGACAGGCTAGAGGGCA |
| | | AAGACTGGAGTGGATCGGTAGAATCGACCCTAATAGCGGCTC |
| | | TACTAAGTATAACGAGAAGTTTAAGAATAGGTTCACTATTAGT |
| | | AGGGATAACTCTAAGAACACCCTGTACCTGCAGATGAATAGC |
| | | CTGAGAGCCGAGGACACCGCCGTCTACTACTGCGCTAGAGACT |
| | | ATAGAAAGGGCCTGTACGCTATGGACTACTGGGGTCAAGGCA |
| | | CTACCGTGACCGTGTCTTCA |
| SEQ ID NO: 608 | Heavy | EVQLVQSGAEVKKPGATVKISCKVSGYTFTSYWMYWVRQARGQ |
| | chain | RLEWIGRIDPNSGSTKYNEKFKNRFTISRDNSKNTLYLQMNSLRA |
| | | EDTAVYYCARDYRKGLYAMDYWGQGTTVTVSSASTKGPSVFPL |
| | | APCSRSTSESTAALGCLVKDYFPEPVTVSWNSGALTSGVHTFPAV |
| | | LQSSGLYSLSSVVTVPSSSLGTKTYTCNVDHKPSNTKVDKRVESK |
| | | YGPPCPPCPAPEFLGGPSVFLFPPKPKDTLMISRTPEVTCVVVDVS |
| | | QEDPEVQFNWYVDGVEVHNAKTKPREEQFNSTYRVVSVLTVLH |
| | | QDWLNGKEYKCKVSNKGLPSSIEKTISKAKGQPREPQVYTLPPSQ |
| | | EEMTKNQVSLTCLVKGFYPSDIAVEWESNGQPENNYKTTPPVLD |
| | | SDGSFFLYSRLTVDKSRWQEGNVFSCSVMHEALHNHYTQKSLSL |
| | | SLG |
| SEQ ID NO: 615 | DNA | GAAGTGCAGCTGGTGCAGTCAGGCGCCGAAGTGAAGAAACCC |
| | heavy | GGCGCTACCGTGAAGATTAGCTGTAAAGTCTCAGGCTACACCT |
| | chain | TCACTAGCTACTGGATGTACTGGGTCCGACAGGCTAGAGGGCA |
| | | AAGACTGGAGTGGATCGGTAGAATCGACCCTAATAGCGGCTC |
| | | TACTAAGTATAACGAGAAGTTTAAGAATAGGTTCACTATTAGT |
| | | AGGGATAACTCTAAGAACACCCTGTACCTGCAGATGAATAGC |
| | | CTGAGAGCCGAGGACACCGCCGTCTACTACTGCGCTAGAGACT |
| | | ATAGAAAGGGCCTGTACGCTATGGACTACTGGGGTCAAGGCA |
| | | CTACCGTGACCGTGTCTTCAGCTAGCACTAAGGGCCCGTCCGT |
| | | GTTCCCCCTGGCACCTTGTAGCCGGAGCACTAGCGAATCCACC |
| | | GCTGCCCTCGGCTGCCTGGTCAAGGATTACTTCCCGGAGCCCG |
| | | TGACCGTGTCCTGGAACAGCGGAGCCCTGACCTCCGGAGTGCA |
| | | CACCTTCCCCGCTGTGCTGCAGAGCTCCGGGCTGTACTCGCTG |
| | | TCGTCGGTGGTCACGGTGCCTTCATCTAGCCTGGGTACCAAGA |
| | | CCTACACTTGCAACGTGGACCACAAGCCTTCCAACACTAAGGT |
| | | GGACAAGCGCGTCGAATCGAAGTACGGCCCACCGTGCCCGCC |
| | | TTGTCCCGCGCCGGAGTTCCTCGGCGGTCCCTCGGTCTTTCTGT |
| | | TCCCACCGAAGCCCAAGGACACTTTGATGATTTCCCGCACCCC |
| | | TGAAGTGACATGCGTGGTCGTGGACGTGTCACAGGAAGATCC |
| | | GGAGGTGCAGTTCAATTGGTACGTGGATGGCGTCGAGGTGCA |
| | | CAACGCCAAAACCAAGCCGAGGGAGGAGCAGTTCAACTCCAC |
| | | TTACCGCGTCGTGTCCGTGCTGACGGTGCTGCATCAGGACTGG |
| | | CTGAACGGGAAGGAGTACAAGTGCAAAGTGTCCAACAAGGGA |
| | | CTTCCTAGCTCAATCGAAAAGACCATCTCGAAAGCCAAGGGA |
| | | CAGCCCCGGGAACCCCAAGTGTATACCCTGCCACCGAGCCAG |
| | | GAAGAAATGACTAAGAACCAAGTCTCATTGACTTGCCTTGTGA |
| | | AGGGCTTCTACCCATCGGATATCGCCGTGGAATGGGAGTCCAA |
| | | CGGCCAGCCGGAAAACAACTACAAGACCACCCCTCCGGTGCT |
| | | GGACTCAGACGGATCCTTCTTCCTCTACTCGCGGCTGACCGTG |
| | | GATAAGAGCAGATGGCAGGAGGGAAATGTGTTCAGCTGTTCT |
| | | GTGATGCATGAAGCCCTGCACAACCACTACACTCAGAAGTCCC |
| | | TGTCCCTCTCCCTGGGA |
| BAP058-Clone O LC | | |
| SEQ ID NO: 609 (Kabat) | LCDR1 | KASQDVGTAVA |
| SEQ ID NO: 610 (Kabat) | LCDR2 | WASTRHT |
| SEQ ID NO: 611 (Kabat) | LCDR3 | QQYNSYPLT |
| SEQ ID NO: 612 | LCDR1 | SQDVGTA |
| (Chothia) | | |
| SEQ ID NO: 613 | LCDR2 | WAS |
| (Chothia) | | |
| SEQ ID NO: 614 | LCDR3 | YNSYPL |
| (Chothia) | | |
| SEQ ID NO: 616 | VL | AIQLTQSPSSLSASVGDRVTITCKASQDVGTAVAWYLQKPGQSPQ |
| | | LLIYWASTRHTGVPSRFSGSGSGTDFTFTISSLEAEDAATYYCQQY |
| | | NSYPLTFGQGTKVEIK |
| SEQ ID NO: 617 | DNA VL | GCTATTCAGCTGACTCAGTCACCTAGTAGCCTGAGCGCTAGTG |
| | | TGGGCGATAGAGTGACTATCACCTGTAAAGCCTCTCAGGACGT |
| | | GGGCACCGCCGTGGCCTGGTATCTGCAGAAGCCTGGTCAATCA |
| | | CCTCAGCTGCTGATCTACTGGGCCTCTACTAGACACACCGGCG |
| | | TGCCCTCTAGGTTTAGCGGTAGCGGTAGTGGCACCGACTTCAC |
| | | CTTCACTATCTCTTCACTGGAAGCCGAGGACGCCGCTACCTAC |
| | | TACTGTCAGCAGTATAATAGCTACCCCCTGACCTTCGGTCAAG |
| | | GCACTAAGGTCGAGATTAAG |
| SEQ ID NO: 618 | Light | AIQLTQSPSSLSASVGDRVTITCKASQDVGTAVAWYLQKPGQSPQ |
| | chain | LLIYWASTRHTGVPSRFSGSGSGTDFTFTISSLEAEDAATYYCQQY |
| | | NSYPLTFGQGTKVEIKRTVAAPSVFIFPPSDEQLKSGTASVVCLLN |
| | | NFYPREAKVQWKVDNALQSGNSQESVTEQDSKDSTYSLSSTLTL |
| | | SKADYEKHKVYACEVTHQGLSSPVTKSFNRGEC |
| SEQ ID NO: 619 | DNA light | GCTATTCAGCTGACTCAGTCACCTAGTAGCCTGAGCGCTAGTG |
| | chain | TGGGCGATAGAGTGACTATCACCTGTAAAGCCTCTCAGGACGT |
| | | GGGCACCGCCGTGGCCTGGTATCTGCAGAAGCCTGGTCAATCA |
| | | CCTCAGCTGCTGATCTACTGGGCCTCTACTAGACACACCGGCG |
| | | TGCCCTCTAGGTTTAGCGGTAGCGGTAGTGGCACCGACTTCAC |
| | | CTTCACTATCTCTTCACTGGAAGCCGAGGACGCCGCTACCTAC |
| | | TACTGTCAGCAGTATAATAGCTACCCCCTGACCTTCGGTCAAG |
| | | GCACTAAGGTCGAGATTAAGCGTACGGTGGCCGCTCCCAGCGT |
| | | GTTCATCTTCCCCCCCAGCGACGAGCAGCTGAAGAGCGGCACC |
| | | GCCAGCGTGGTGTGCCTGCTGAACAACTTCTACCCCCGGGAGG |
| | | CCAAGGTGCAGTGGAAGGTGGACAACGCCCTGCAGAGCGGCA |
| | | ACAGCCAGGAGAGCGTCACCGAGCAGGACAGCAAGGACTCCA |
| | | CCTACAGCCTGAGCAGCACCCTGACCCTGAGCAAGGCCGACT |
| | | ACGAGAAGCATAAGGTGTACGCCTGCGAGGTGACCCACCAGG |
| | | GCCTGTCCAGCCCCGTGACCAAGAGCTTCAACAGGGGCGAGT |
| | | GC |
| BAP058-Clone N HC | | |
| SEQ ID NO: 601 (Kabat) | HCDR1 | SYWMY |
| SEQ ID NO: 602 (Kabat) | HCDR2 | RIDPNSGSTKYNEKFKN |
| SEQ ID NO: 603 (Kabat) | HCDR3 | DYRKGLYAMDY |
| SEQ ID NO: 604 | HCDR1 | GYTFTSY |
| (Chothia) | | |
| SEQ ID NO: 605 | HCDR2 | DPNSGS |
| (Chothia) | | |
| SEQ ID NO: 603 | HCDR3 | DYRKGLYAMDY |
| (Chothia) | | |
| SEQ ID NO: 620 | VH | EVQLVQSGAEVKKPGATVKISCKVSGYTFTSYWMYWVRQATGQ |
| | | GLEWMGRIDPNSGSTKYNEKFKNRVTITADKSTSTAYMELSSLRS |
| | | EDTAVYYCARDYRKGLYAMDYWGQGTTVTVSS |
| SEQ ID NO: 621 | DNA VH | GAAGTGCAGCTGGTGCAGTCAGGCGCCGAAGTGAAGAAACCC |
| | | GGCGCTACCGTGAAGATTAGCTGTAAAGTCTCAGGCTACACCT |
| | | TCACTAGCTACTGGATGTACTGGGTCCGACAGGCTACCGGTCA |
| | | AGGCCTGGAGTGGATGGGTAGAATCGACCCTAATAGCGGCTC |
| | | TACTAAGTATAACGAGAAGTTTAAGAATAGAGTGACTATCACC |
| | | GCCGATAAGTCTACTAGCACCGCCTATATGGAACTGTCTAGCC |
| | | TGAGATCAGAGGACACCGCCGTCTACTACTGCGCTAGAGACTA |
| | | TAGAAAGGGCCTGTACGCTATGGACTACTGGGGTCAAGGCAC |
| | | TACCGTGACCGTGTCTTCA |
| SEQ ID NO: 622 | Heavy | EVQLVQSGAEVKKPGATVKISCKVSGYTFTSYWMYWVRQATGQ |
| | chain | GLEWMGRIDPNSGSTKYNEKFKNRVTITADKSTSTAYMELSSLRS |
| | | EDTAVYYCARDYRKGLYAMDYWGQGTTVTVSSASTKGPSVFPL |
| | | APCSRSTSESTAALGCLVKDYFPEPVTVSWNSGALTSGVHTFPAV |
| | | LQSSGLYSLSSVVTVPSSSLGTKTYTCNVDHKPSNTKVDKRVESK |
| | | YGPPCPPCPAPEFLGGPSVFLFPPKPKDTLMISRTPEVTCVVVDV |
| | | SQEDPEVQFNWYVDGVEVHNAKTKPREEQFNSTYRVVSVLTVLH |
| | | QDWLNGKEYKCKVSNKGLPSSIEKTISKAKGQPREPQVYTLPPSQ |
| | | EEMTKNQVSLTCLVKGFYPSDIAVEWESNGQPENNYKTTPPVLD |
| | | SDGSFFLYSRLTVDKSRWQEGNVFSCSVMHEALHNHYTQKSLSL |
| | | SLG |
| SEQ ID NO: 623 | DNA | GAAGTGCAGCTGGTGCAGTCAGGCGCCGAAGTGAAGAAACCC |
| | heavy | GGCGCTACCGTGAAGATTAGCTGTAAAGTCTCAGGCTACACCT |
| | chain | TCACTAGCTACTGGATGTACTGGGTCCGACAGGCTACCGGTCA |
| | | AGGCCTGGAGTGGATGGGTAGAATCGACCCTAATAGCGGCTC |
| | | TACTAAGTATAACGAGAAGTTTAAGAATAGAGTGACTATCACC |
| | | GCCGATAAGTCTACTAGCACCGCCTATATGGAACTGTCTAGCC |
| | | TGAGATCAGAGGACACCGCCGTCTACTACTGCGCTAGAGACTA |
| | | TAGAAAGGGCCTGTACGCTATGGACTACTGGGGTCAAGGCAC |
| | | TACCGTGACCGTGTCTTCAGCTAGCACTAAGGGCCCGTCCGTG |
| | | TTCCCCCTGGCACCTTGTAGCCGGAGCACTAGCGAATCCACCG |
| | | CTGCCCTCGGCTGCCTGGTCAAGGATTACTTCCCGGAGCCCGT |
| | | GACCGTGTCCTGGAACAGCGGAGCCCTGACCTCCGGAGTGCA |
| | | CACCTTCCCCGCTGTGCTGCAGAGCTCCGGGCTGTACTCGCTG |
| | | TCGTCGGTGGTCACGGTGCCTTCATCTAGCCTGGGTACCAAGA |
| | | CCTACACTTGCAACGTGGACCACAAGCCTTCCAACACTAAGGT |
| | | GGACAAGCGCGTCGAATCGAAGTACGGCCCACCGTGCCCGCC |
| | | TTGTCCCGCGCCGGAGTTCCTCGGCGGTCCCTCGGTCTTTCTGT |
| | | TCCCACCGAAGCCCAAGGACACTTTGATGATTTCCCGCACCCC |
| | | TGAAGTGACATGCGTGGTCGTGGACGTGTCACAGGAAGATCC |
| | | GGAGGTGCAGTTCAATTGGTACGTGGATGGCGTCGAGGTGCA |
| | | CAACGCCAAAACCAAGCCGAGGGAGGAGCAGTTCAACTCCAC |
| | | TTACCGCGTCGTGTCCGTGCTGACGGTGCTGCATCAGGACTGG |
| | | CTGAACGGGAAGGAGTACAAGTGCAAAGTGTCCAACAAGGGA |
| | | CTTCCTAGCTCAATCGAAAAGACCATCTCGAAAGCCAAGGGA |
| | | CAGCCCCGGGAACCCCAAGTGTATACCCTGCCACCGAGCCAG |
| | | GAAGAAATGACTAAGAACCAAGTCTCATTGACTTGCCTTGTGA |
| | | AGGGCTTCTACCCATCGGATATCGCCGTGGAATGGGAGTCCAA |
| | | CGGCCAGCCGGAAAACAACTACAAGACCACCCCTCCGGTGCT |
| | | GGACTCAGACGGATCCTTCTTCCTCTACTCGCGGCTGACCGTG |
| | | GATAAGAGCAGATGGCAGGAGGGAAATGTGTTCAGCTGTTCT |
| | | GTGATGCATGAAGCCCTGCACAACCACTACACTCAGAAGTCCC |
| | | TGTCCCTCTCCCTGGGA |
| BAP058-Clone N LC | | |
| SEQ ID NO: 609 (Kabat) | LCDR1 | KASQDVGTAVA |
| SEQ ID NO: 610 (Kabat) | LCDR2 | WASTRHT |
| SEQ ID NO: 611 (Kabat) | LCDR3 | QQYNSYPLT |
| SEQ ID NO: 612 | LCDR1 | SQDVGTA |
| (Chothia) | | |
| SEQ ID NO: 613 | LCDR2 | WAS |
| (Chothia) | | |
| SEQ ID NO: 614 | LCDR3 | YNSYPL |
| (Chothia) | | |
| SEQ ID NO: 624 | VL | DVVMTQSPLSLPVTLGQPASISCKASQDVGTAVAWYQQKPGQAP |
| | | RLLIYWASTRHTGVPSRFSGSGSGTEFTLTISSLQPDDFATYYC |
| | | QQYNSYPLTFGQGTKVEIK |
| SEQ ID NO: 625 | DNA VL | GACGTCGTGATGACTCAGTCACCCCTGAGCCTGCCCGTGACCC |
| | | TGGGGCAGCCCGCCTCTATTAGCTGTAAAGCCTCTCAGGACGT |
| | | GGGCACCGCCGTGGCCTGGTATCAGCAGAAGCCAGGGCAAGC |
| | | CCCTAGACTGCTGATCTACTGGGCCTCTACTAGACACACCGGC |
| | | GTGCCCTCTAGGTTTAGCGGTAGCGGTAGTGGCACCGAGTTCA |
| | | CCCTGACTATCTCTTCACTGCAGCCCGACGACTTCGCTACCTAC |
| | | TACTGTCAGCAGTATAATAGCTACCCCCTGACCTTCGGTCAAG |
| | | GCACTAAGGTCGAGATTAAG |
| SEQ ID NO: 626 | Light chain | DVVMTQSPLSLPVTLGQPASISCKASQDVGTAVAWYQQKPGQAPR |
| | | LLIYWASTRHTGVPSRFSGSGSGTEFTLTISSLQPDDFATYYCQQ |
| | | YNSYPLTFGQGTKVEIKRTVAAPSVFIFPPSDEQLKSGTASVVCL |
| | | LNNFYPREAKVQWKVDNALQSGNSQESVTEQDSKDSTYSLSSTLT |
| | | LSKADYEKHKVYACEVTHQGLSSPVTKSFNRGEC |
| SEQ ID NO: 627 | DNA light | GACGTCGTGATGACTCAGTCACCCCTGAGCCTGCCCGTGACCC |
| | chain | TGGGGCAGCCCGCCTCTATTAGCTGTAAAGCCTCTCAGGACGT |
| | | GGGCACCGCCGTGGCCTGGTATCAGCAGAAGCCAGGGCAAGC |
| | | CCCTAGACTGCTGATCTACTGGGCCTCTACTAGACACACCGGC |
| | | GTGCCCTCTAGGTTTAGCGGTAGCGGTAGTGGCACCGAGTTCA |
| | | CCCTGACTATCTCTTCACTGCAGCCCGACGACTTCGCTACCTAC |
| | | TACTGTCAGCAGTATAATAGCTACCCCCTGACCTTCGGTCAAG |
| | | GCACTAAGGTCGAGATTAAGCGTACGGTGGCCGCTCCCAGCGT |
| | | GTTCATCTTCCCCCCCAGCGACGAGCAGCTGAAGAGCGGCACC |
| | | GCCAGCGTGGTGTGCCTGCTGAACAACTTCTACCCCCGGGAGG |
| | | CCAAGGTGCAGTGGAAGGTGGACAACGCCCTGCAGAGCGGCA |
| | | ACAGCCAGGAGAGCGTCACCGAGCAGGACAGCAAGGACTCCA |
| | | CCTACAGCCTGAGCAGCACCCTGACCCTGAGCAAGGCCGACT |
| | | ACGAGAAGCATAAGGTGTACGCCTGCGAGGTGACCCACCAGG |
| | | GCCTGTCCAGCCCCGTGACCAAGAGCTTCAACAGGGGCGAGT |
| | | GC |
| BAP058-Clone O HC | | |
| SEQ ID NO: 628 (Kabat) | HCDR1 | agctactggatgtac |
| SEQ ID NO: 629 (Kabat) | HCDR2 | agaatcgaccctaatagcggctctactaagtat |
| | | aacgagaagtttaagaat |
| SEQ ID NO: 630 (Kabat) | HCDR3 | gactatagaaagggcctgtacgctatggactac |
| SEQ ID NO: 631 | HCDR1 | ggctacaccttcactagctac |
| (Chothia) | | |
| SEQ ID NO: 632 | HCDR2 | gaccctaatagcggctct |
| (Chothia) | | |
| SEQ ID NO: 630 | HCDR3 | gactatagaaagggcctgtacgctatggactac |
| (Chothia) | | |
| BAP058-Clone O LC | | |
| SEQ ID NO: 633 (Kabat | LCDR1 | aaagcctctcaggacgtgggcaccgccgtggcc |
| SEQ ID NO: 634 (Kabat) | LCDR2 | tgggcctctactagacacacc |
| SEQ ID NO: 635 (Kabat) | LCDR3 | cagcagtataatagctaccccctgacc |
| SEQ ID NO: 636 | LCDR1 | tctcaggacgtgggcaccgcc |
| (Chothia) | | |
| SEQ ID NO: 637 | LCDR2 | tgggcctct |
| (Chothia) | | |
| SEQ ID NO: 638 | LCDR3 | tataatagctaccccctg |
| (Chothia) | | |
| BAP058-Clone N HC | | |
| SEQ ID NO: 628 (Kabat) | HCDR1 | agctactggatgtac |
| SEQ ID NO: 629 (Kabat) | HCDR2 | agaatcgaccctaatagcggctctactaagta |
| | | taacgagaagtttaagaat |
| SEQ ID NO: 630 (Kabat) | HCDR3 | gactatagaaagggcctgtacgctatggactac |
| SEQ ID NO: 631 | HCDR1 | ggctacaccttcactagctac |
| (Chothia) | | |
| SEQ ID NO: 632 | HCDR2 | gaccctaatagcggctct |
| (Chothia) | | |
| SEQ ID NO: 630 | HCDR3 | gactatagaaagggcctgtacgctatggactac |
| (Chothia) | | |
| BAP058-Clone N LC | | |
| SEQ ID NO: 633 (Kabat) | LCDR1 | aaagcctctcaggacgtgggcaccgccgtggcc |
| SEQ ID NO: 634 (Kabat) | LCDR2 | tgggcctctactagacacacc |
| SEQ ID NO: 635 (Kabat) | LCDR3 | cagcagtataatagctaccccctgacc |
| SEQ ID NO: 636 | LCDR1 | tctcaggacgtgggcaccgcc |
| (Chothia) | | |
| SEQ ID NO: 637 | LCDR2 | tgggcctct |
| (Chothia) | | |
| SEQ ID NO: 638 | LCDR3 | tataatagctaccccctg |
| (Chothia) | | |

### Other Exemplary PD-L1 Inhibitors

In one embodiment, the anti-PD-L1 antibody molecule is Atezolizumab (Genentech/Roche), also known as MPDL3280A, RG7446, R05541267, YW243.55.S70, or TECENTRIQ™. Atezolizumab and other anti-PD-L1 antibodies are disclosed in U.S. Pat. No. 8,217,149, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Atezolizuma, e.g., as disclosed in Table 8.

In one embodiment, the anti-PD-L1 antibody molecule is Avelumab (Merck Serono and Pfizer), also known as MSB0010718C. Avelumab and other anti-PD-L1 antibodies are disclosed in WO 2013/079174, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Avelumab, e.g., as disclosed in Table 8.

In one embodiment, the anti-PD-L1 antibody molecule is Durvalumab (MedImmune/AstraZeneca), also known as MEDI4736. Durvalumab and other anti-PD-L1 antibodies are disclosed in U.S. Pat. No. 8,779,108, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Durvalumab, e.g., as disclosed in Table 8.

In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety. In one embodiment, the anti-PD-L1 antibody molecule comprises one or more of the CDR sequences (or collectively all of the CDR sequences), the heavy chain or light chain variable region sequence, or the heavy chain or light chain sequence of BMS-936559, e.g., as disclosed in Table 8.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

In one embodiment, the anti-PD-L1 antibody is an antibody that competes for binding with, and/or binds to the same epitope on PD-L1 as, one of the anti-PD-L1 antibodies described herein.

TABLE 8
Amino acid sequences of other exemplary
anti-PD-L1 antibody molecules

| | | |
|---|---|---|
| Atezolizumab | | |
| SEQ ID NO: 639 | Heavy | EVQLVESGGGLVQPGGSLRLSCAASG |
| | chain | FTFSDSWIHWVRQAPGKGLEWVAWIS |
| | | PYGGSTYYADSVKGRFTISADTSKNT |
| | | AYLQMNSLRAEDTAVYYCARRHWPGG |
| | | FDYWGQGTLVTVSSASTKGPSVFPLA |
| | | PSSKSTSGGTAALGCLVKDYFPEPVT |
| | | VSWNSGALTSGVHTFPAVLQSSGLYS |
| | | LSSVVTVPSSSLGTQTYICNVNHKPS |
| | | NTKVDKKVEPKSCDKTHTCPPCPAPE |
| | | LLGGPSVFLFPPKPKDTLMISRTPEV |
| | | TCVVVDVSHEDPEVKFNWYVDGVEVH |
| | | NAKTKPREEQYASTYRVVSVLTVLHQ |
| | | DWLNGKEYKCKVSNKALPAPIEKTIS |
| | | KAKGQPREPQVYTLPPSREEMTKNQV |
| | | SLTCLVKGFYPSDIAVEWESNGQPEN |
| | | NYKTTPPVLDSDGSFFLYSKLTVDKS |
| | | RWQQGNVFSCSVMHEALHNHYTQKSL |
| | | SLSPGK |
| SEQ ID NO: 640 | Light | DIQMTQSPSSLSASVGDRVTITCRAS |
| | chain | QDVSTAVAWYQQKPGKAPKLLIYSAS |
| | | FLYSGVPSRFSGSGSGTDFTLTISSL |
| | | QPEDFATYYCQQYLYHPATFGQGTKV |
| | | EIKRTVAAPSVFIFPPSDEQLKSGTA |
| | | SVVCLLNNFYPREAKVQWKVDNALQS |
| | | GNSQESVTEQDSKDSTYSLSSTLTLS |
| | | KADYEKHKVYACEVTHQGLSSPVTKS |
| | | FNRGEC |
| Avelumab | | |
| SEQ ID NO: 641 | Heavy | EVQLLESGGGLVQPGGSLRLSCAASG |
| | chain | FTFSSYIMMWVRQAPGKGLEWVSSIY |
| | | PSGGITFYADTVKGRFTISRDNSKNT |
| | | LYLQMNSLRAEDTAVYYCARIKLGTV |
| | | TTVDYWGQGTLVTVSSASTKGPSVFP |
| | | LAPSSKSTSGGTAALGCLVKDYFPEP |
| | | VTVSWNSGALTSGVHTFPAVLQSSGL |
| | | YSLSSVVTVPSSSLGTQTYICNVNHK |
| | | PSNTKVDKKVEPKSCDKTHTCPPCPA |
| | | PELLGGPSVFLFPPKPKDTLMISRTP |
| | | EVTCVVVDVSHEDPEVKFNWYVDGVE |
| | | VHNAKTKPREEQYNSTYRVVSVLTVL |
| | | HQDWLNGKEYKCKVSNKALPAPIEKT |
| | | ISKAKGQPREPQVYTLPPSRDELTKN |
| | | QVSLTCLVKGFYPSDIAVEWESNGQP |
| | | ENNYKTTPPVLDSDGSFFLYSKLTVD |
| | | KSRWQQGNVFSCSVMHEALHNHYTQK |
| | | SLSLSPGK |
| SEQ ID NO: 642 | Light | QSALTQPASVSGSPGQSITISCTGTS |
| | chain | SDVGGYNYVSWYQQHPGKAPKLMIYD |
| | | VSNRPSGVSNRFSGSKSGNTASLTIS |
| | | GLQAEDEADYYCSSYTSSSTRVFGTG |
| | | TKVTVLGQPKANPTVTLFTPSSEELQ |
| | | ANKATLVCLISDFTPGAVTVAWKADG |
| | | SPVKAGVETTKPSKQSNNKYAASSYL |
| | | SLTPEQWKSHRSYSCQVTHEGSTVEK |
| | | TVAPTECS |
| Durvalumab | | |
| SEQ ID NO: 643 | Heavy | EVQLVESGGGLVQPGGSLRLSCAASG |
| | chain | FTFSRYWMSWVRQAPGKGLEWVANIK |
| | | QDGSEKYYVDSVKGRFTISRDNAKNS |
| | | LYLQMNSLRAEDTAVYYCAREGGWFG |
| | | ELAFDYWGQGTLVTVSSASTKGPSVF |
| | | PLAPSSKSTSGGTAALGCLVKDYFPE |
| | | PVTVSWNSGALTSGVHTFPAVLQSSG |
| | | LYSLSSVVTVPSSSLGTQTYICNVNH |
| | | KPSNTKVDKRVEPKSCDKTHTCPPCP |
| | | APEFEGGPSVFLFPPKPKDTLMISR |
| | | TPEVTCVVVDVSHEDPEVKFNWYVDG |
| | | VEVHNAKTKPREEQYNSTYRVVSVLT |
| | | VLHQDWLNGKEYKCKVSNKALPASIE |
| | | KTISKAKGQPREPQVYTLPPSREMTK |
| | | NQVSLTCLVKGFYPSDIAVEWESNGQ |
| | | PENNYKTTPPVLDSDGSFFLYSLTVD |
| | | KSRWQQGNVFSCSVMHEALHNHYTQK |
| | | SLSLSPGK |
| SEQ ID NO: 644 | Light | EIVLTQSPGTLSLSPGERATLSCRAS |
| | chain | QRVSSSYLAWYQQKPGQAPRLLIYDA |
| | | SRATGIPDRFSGSGSGTDFrLTISRL |
| | | EPEDFAVYYCQQYGSLPWTFGQGTKV |
| | | KRTVAAPSVFIFPPSDEQLKSGTASV |
| | | VCLLNNFYPREAKVQWKVDNALQSGS |
| | | QESVTEQDSKDSTYSLSSTLTLSKAD |
| | | YEKHKVYACEVTHQGLSSPVTKSFNG |
| | | EC |
| BMS-936559 | | |
| SEQ ID NO: 645 | VH | QVQLVQSGAEVKKPGSSVKVSCKTSG |
| | | DTFSTYAISWVRQAPGQGLEWMGGPI |
| | | FGKAHYAQKFQGRVTITADESTSTAY |
| | | MELSSLRSEDTAVYFCARKFHFVSPF |
| | | GMDVWGQGTTVTVSS |
| SEQ ID NO: 646 | VL | EIVLTQSPATLSLSPGERATLSCRAS |
| | | QSVSSYLAWYQQKPGQAPRLLIYDAS |
| | | NRATGIPARFSGSGSGTDFTLTISSL |
| | | EPEDFAVYYCQQRSNWPTFGQGTKVE |
| | | IK |

### Exemplary LAG-3 Inhibitors

In certain embodiments, the anti-CD73 molecule described herein is administered in combination with a LAG-3 inhibitor known in the art. The LAG-3 inhibitor may be an antibody, an antigen binding fragment thereof, an immunoadhesin, a fusion protein, or oligopeptide. In some embodiments, the LAG-3 inhibitor is chosen from LAG525 (Novartis), BMS-986016 (Bristol-Myers Squibb), TSR-033 (Tesaro), MK-4280 (Merck & Co), or REGN3767 (Regeneron).

### Exemplary Anti-LAG-3 Antibody Molecules

In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated by reference in its entirety.

In one embodiment, the anti-LAG-3 antibody molecule comprises at least one, two, three, four, five or six complementarity determining regions (CDRs) (or collectively all of the CDRs) from a heavy and light chain variable region comprising an amino acid sequence shown in Table 9 (e.g., from the heavy and light chain variable region sequences of BAP050-Clone I or BAP050-Clone J disclosed in Table 9), or encoded by a nucleotide sequence shown in Table 9. In some embodiments, the CDRs are according to the Kabat definition (e.g., as set out in Table 9). In some embodiments, the CDRs are according to the Chothia definition (e.g., as set out in Table 5). In some embodiments, the CDRs are according to the combined CDR definitions of both Kabat and Chothia (e.g., as set out in Table 9). In one embodiment, the combination of Kabat and Chothia CDR of VH CDR1 comprises the amino acid sequence GFTLTNYGMN (SEQ ID NO: 766). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 9, or encoded by a nucleotide sequence shown in Table 9.

In one embodiment, the anti-LAG-3 antibody molecule comprises a heavy chain variable region (VH) comprising a VHCDR1 amino acid sequence of SEQ ID NO: 701, a VHCDR2 amino acid sequence of SEQ ID NO: 702, and a VHCDR3 amino acid sequence of SEQ ID NO: 703; and a light chain variable region (VL) comprising a VLCDR1 amino acid sequence of SEQ ID NO: 710, a VLCDR2 amino acid sequence of SEQ ID NO: 711, and a VLCDR3 amino acid sequence of SEQ ID NO: 712, each disclosed in Table 9.

In one embodiment, the anti-LAG-3 antibody molecule comprises a VH comprising a VHCDR1 encoded by the nucleotide sequence of SEQ ID NO: 736 or 737, a VHCDR2 encoded by the nucleotide sequence of SEQ ID NO: 738 or 739, and a VHCDR3 encoded by the nucleotide sequence of SEQ ID NO: 740 or 741; and a VL comprising a VLCDR1 encoded by the nucleotide sequence of SEQ ID NO: 746 or 747, a VLCDR2 encoded by the nucleotide sequence of SEQ ID NO: 748 or 749, and a VLCDR3 encoded by the nucleotide sequence of SEQ ID NO: 750 or 751, each disclosed in Table 9. In one embodiment, the anti-LAG-3 antibody molecule comprises a VH comprising a VHCDR1 encoded by the nucleotide sequence of SEQ ID NO: 758 or 737, a VHCDR2 encoded by the nucleotide sequence of SEQ ID NO: 759 or 739, and a VHCDR3 encoded by the nucleotide sequence of SEQ ID NO: 760 or 741; and a VL comprising a VLCDR1 encoded by the nucleotide sequence of SEQ ID NO: 746 or 747, a VLCDR2 encoded by the nucleotide sequence of SEQ ID NO: 748 or 749, and a VLCDR3 encoded by the nucleotide sequence of SEQ ID NO: 750 or 751, each disclosed in Table 9.

In one embodiment, the anti-LAG-3 antibody molecule comprises a VH comprising the amino acid sequence of SEQ ID NO: 706, or an amino acid sequence having at least about 85%, 90%, 95%, or 99% sequence identity to SEQ ID NO: 706. In one embodiment, the anti-LAG-3 antibody molecule comprises a VL comprising the amino acid sequence of SEQ ID NO: 718, or an amino acid sequence having at least about 85%, 90%, 95%, or 99% sequence identity to SEQ ID NO: 718. In one embodiment, the anti-LAG-3 antibody molecule comprises a VH comprising the amino acid sequence of SEQ ID NO: 724, or an amino acid sequence having at least about 85%, 90%, 95%, or 99% sequence identity to SEQ ID NO: 724. In one embodiment, the anti-LAG-3 antibody molecule comprises a VL comprising the amino acid sequence of SEQ ID NO: 730, or an amino acid sequence having at least about 85%, 90%, 95%, or 99% sequence identity to SEQ ID NO: 730. In one embodiment, the anti-LAG-3 antibody molecule comprises a VH comprising the amino acid sequence of SEQ ID NO: 706 and a VL comprising the amino acid sequence of SEQ ID NO: 718. In one embodiment, the anti-LAG-3 antibody molecule comprises a VH comprising the amino acid sequence of SEQ ID NO: 724 and a VL comprising the amino acid sequence of SEQ ID NO: 730.

In one embodiment, the antibody molecule comprises a VH encoded by the nucleotide sequence of SEQ ID NO: 707 or 708, or a nucleotide sequence having at least about 85%, 90%, 95%, or 99% sequence identity to SEQ ID NO: 707 or 708. In one embodiment, the antibody molecule comprises a VL encoded by the nucleotide sequence of SEQ ID NO: 719 or 720, or a nucleotide sequence having at least about 85%, 90%, 95%, or 99% sequence identity to SEQ ID NO: 719 or 720. In one embodiment, the antibody molecule comprises a VH encoded by the nucleotide sequence of SEQ ID NO: 725 or 726, or a nucleotide sequence having at least about 85%, 90%, 95%, or 99% sequence identity to SEQ ID NO: 725 or 726. In one embodiment, the antibody molecule comprises a VL encoded by the nucleotide sequence of SEQ ID NO: 731 or 732, or a nucleotide sequence having at least about 85%, 90%, 95%, or 99% sequence identity to SEQ ID NO: 731 or 732. In one embodiment, the antibody molecule comprises a VH encoded by the nucleotide sequence of SEQ ID NO: 707 or 708 and a VL encoded by the nucleotide sequence of SEQ ID NO: 719 or 720. In one embodiment, the antibody molecule comprises a VH encoded by the nucleotide sequence of SEQ ID NO: 725 or 726 and a VL encoded by the nucleotide sequence of SEQ ID NO: 731 or 732.

In one embodiment, the anti-LAG-3 antibody molecule comprises a heavy chain comprising the amino acid sequence of SEQ ID NO: 709, or an amino acid sequence having at least about 85%, 90%, 95%, or 99% sequence identity to SEQ ID NO: 709. In one embodiment, the anti-LAG-3 antibody molecule comprises a light chain comprising the amino acid sequence of SEQ ID NO: 721, or an amino acid sequence having at least about 85%, 90%, 95%, or 99% sequence identity to SEQ ID NO: 721. In one embodiment, the anti-LAG-3 antibody molecule comprises a heavy chain comprising the amino acid sequence of SEQ ID NO: 727, or an amino acid sequence having at least about 85%, 90%, 95%, or 99% sequence identity to SEQ ID NO: 727. In one embodiment, the anti-LAG-3 antibody molecule comprises a light chain comprising the amino acid sequence of SEQ ID NO: 733, or an amino acid sequence having at least about 85%, 90%, 95%, or 99% sequence identity to SEQ ID NO: 733. In one embodiment, the anti-LAG-3 antibody molecule comprises a heavy chain comprising the amino acid sequence of SEQ ID NO: 709 and a light chain comprising the amino acid sequence of SEQ ID NO: 721. In one embodiment, the anti-LAG-3 antibody molecule comprises a heavy chain comprising the amino acid sequence of SEQ ID NO: 727 and a light chain comprising the amino acid sequence of SEQ ID NO: 733.

In one embodiment, the antibody molecule comprises a heavy chain encoded by the nucleotide sequence of SEQ ID NO: 716 or 717, or a nucleotide sequence having at least about 85%, 90%, 95%, or 99% sequence identity to SEQ ID NO: 716 or 717. In one embodiment, the antibody molecule comprises a light chain encoded by the nucleotide sequence of SEQ ID NO: 722 or 723, or a nucleotide sequence having at least about 85%, 90%, 95%, or 99% sequence identity to SEQ ID NO: 722 or 723. In one embodiment, the antibody molecule comprises a heavy chain encoded by the nucleotide sequence of SEQ ID NO: 728 or 729, or a nucleotide sequence having at least about 85%, 90%, 95%, or 99% sequence identity to SEQ ID NO: 728 or 729. In one embodiment, the antibody molecule comprises a light chain encoded by the nucleotide sequence of SEQ ID NO: 734 or 735, or a nucleotide sequence having at least about 85%, 90%, 95%, or 99% sequence identity to SEQ ID NO: 734 or 735. In one embodiment, the antibody molecule comprises a heavy chain encoded by the nucleotide sequence of SEQ ID NO: 716 or 717 and a light chain encoded by the nucleotide sequence of SEQ ID NO: 722 or 723. In one embodiment, the antibody molecule comprises a heavy chain encoded by the nucleotide sequence of SEQ ID NO: 728 or 729 and a light chain encoded by the nucleotide sequence of SEQ ID NO: 734 or 735.

The antibody molecules described herein can be made by vectors, host cells, and methods described in US 2015/0259420, incorporated by reference in its entirety.

TABLE 9
Amino acid and nucleotide sequences of exemplary
anti-LAG-3 antibody molecules

| | | |
|---|---|---|
| BAP050-Clone I HC | | |
| SEQ ID NO: 701 | HCDR1 | NYGMN |
| (Kabat) | | |
| SEQ ID NO: 702 | HCDR2 | WINTDTGEPTYADDFKG |
| (Kabat) | | |
| SEQ ID NO: 703 | HCDR3 | NPPYYYGTNNAEAMDY |
| (Kabat) | | |
| | HCDR1 | GFTLTNY |
| SEQ ID NO: 704 | | |
| (Chothia) | | |
| SEQ ID NO: 705 | HCDR2 | NTDTGE |
| (Chothia) | | |
| SEQ ID NO: 703 | HCDR3 | NPPYYYGTNNAEAMDY |
| (Chothia) | | |
| SEQ ID NO: 706 | VH | QVQLVQSGAEVKKPGASVKVSCKASGITLTNYGMNWVRQARGQ |
| | | RLEWIGWINTDTGEPTYADDFKCRFVFSLDTSVSTAYLQISSLK |
| | | AEDTAVYYCARNPPYYYGTNNAEAMDYWGQGTTVTVSS |
| SEQ ID NO: 707 | DNA VH | CAAGTGCAGCTGGTGCAGTCGGGAGCCGAAGTGAAGAAGCCTG |
| | | GAGCCTCGGTGAAGGTGTCGTGCAAGGCATCCGGATTCACCCT |
| | | CACCAATTACGGGATGAACTGGGTCAGACAGGCCCGGGGTCAA |
| | | CGGCTGGAGTGGATCGGATGGATTAACACCGACACCGGGGAGC |
| | | CTACCTACGCGGACGATTTCAAGGGACGGTTCGTGTTCTCCCT |
| | | CGACACCTCCGTGTCCACCGCCTACCTCCAAATCTCCTCACTG |
| | | AAAGCGGAGGACACCGCCGTGTACTATTGCGCGAGGAACCCGC |
| | | CCTACTACTACGGAACCAACAACGCCGAAGCCATGGACTACTG |
| | | GGGCCAGGGCACCACTGTGACTGTGTCCAGC |
| SEQ ID NO: 708 | DNA VHC | CAGGTGCAGCTGGTGCAGTCTGGCGCCGAAGTGAAGAAACCTG |
| | | GCGCCTCCGTGAAGGTGTCCTGCAAGGCCTCTGGCTTCACCCT |
| | | GACCAACTACGGCATGAACTGGGTGCGACAGGCCAGGGGCCAG |
| | | GGCTGGAATGGATCGGCTGGATCAACACCGACACCGGCGACCT |
| | | ACCTACGCCGACGACTTCAAGGGCAGATTCGTGTTCTCCCTGG |
| | | ACACCTCCGTGTCCACCGCCTACCTGCAGATCTCCAGCCTGAAG |
| | | GCCGAGGATACCGCCGTGTACTACTGCGCCCGGAACCCCCCTT |
| | | ACTACTACGGCACCAACAACGCCGAGGCCATGGACTATTGGGG |
| | | CCAGGGCACCACCGTGACCGTGTCCTCT |
| SEQ ID NO: 709 | Heavy chain | QVQLVQSGAEVKKPGASVKVSCKASGFTLTNYGMNWVRQARGQR |
| | | LEWIGWINTDTGEPTYADDFKGRFVFSLDTSVSTAYLQISSLKA |
| | | EDTAVYYCARNPPYYYGTNNAEAMDYWGQGTTVTVSSASTKGPS |
| | | VFPLAPCSRSTSESTAALGCLVKDYFPEPVTVSWNSGALTSGVHT |
| | | FPAVLQSSGLYSLSSVVTVPSSSLGTKTYTCNVDHKPSNTKVDKR |
| | | VESKYGPPCPPCPAPEFLGGPSVFLFPPKPKDTLMISRTPEVTCV |
| | | VVDVSQEDPEVQFNWYVDGVEVHNAKTKPREEQFNSTYRVVSVLT |
| | | VLHQDWLNGKEYKCKVSNKGLPSSIEKTISKAKGQPREPQVYTLP |
| | | PSQEEMTKNQVSLTCLVKGFYPSDIAVEWESNGQPENNYKTTPPV |
| | | LDSDGSFFLYSRLTVDKSRWQEGNVFSCSVMHEALHNHYTQKSLS |
| | | LSLG |
| SEQ ID NO: 716 | DNA | CAAGTGCAGCTGGTGCAGTCGGGAGCCGAAGTGAAGAAGCCTG |
| | heavy | GAGCCTCGGTGAAGGTGTCGTGCAAGGCATCCGGATTCACCCT |
| | chain | CACCAATTACGGGATGAACTGGGTCAGACAGGCCCGGGGTCAA |
| | | CGGCTGGAGTGGATCGGATGGATTAACACCGACACCGGGGAGC |
| | | CTACCTACGCGGACGATTTCAAGGGACGGTTCGTGTTCTCCCTC |
| | | GACACCTCCGTGTCCACCGCCTACCTCCAAATCTCCTCACTGAA |
| | | AGCGGAGGACACCGCCGTGTACTATTGCGCGAGGAACCCGCCC |
| | | TACTACTACGGAACCAACAACGCCGAAGCCATGGACTACTGGG |
| | | GCCAGGGCACCACTGTGACTGTGTCCAGCGCGTCCACTAAGGG |
| | | CCCGTCCGTGTTCCCCCTGGCACCTTGTAGCCGGAGCACTAGCG |
| | | AATCCACCGCTGCCCTCGGCTGCCTGGTCAAGGATTACTTCCCG |
| | | GAGCCCGTGACCGTGTCCTGGAACAGCGGAGCCCTGACCTCCG |
| | | GAGTGCACACCTTCCCCGCTGTGCTGCAGAGCTCCGGGCTGTAC |
| | | TCGCTGTCGTCGGTGGTCACGGTGCCTTCATCTAGCCTGGGTAC |
| | | CAAGACCTACACTTGCAACGTGGACCACAAGCCTTCCAACACT |
| | | AAGGTGGACAAGCGCGTCGAATCGAAGTACGGCCCACCGTGCC |
| | | CGCCTTGTCCCGCGCCGGAGTTCCTCGGCGGTCCCTCGGTCTTT |
| | | CTGTTCCCACCGAAGCCCAAGGACACTTTGATGATTTCCCGCAC |
| | | CCCTGAAGTGACATGCGTGGTCGTGGACGTGTCACAGGAAGAT |
| | | CCGGAGGTGCAGTTCAATTGGTACGTGGATGGCGTCGAGGTGC |
| | | ACAACGCCAAAACCAAGCCGAGGGAGGAGCAGTTCAACTCCAC |
| | | TTACCGCGTCGTGTCCGTGCTGACGGTGCTGCATCAGGACTGGC |
| | | TGAACGGGAAGGAGTACAAGTGCAAAGTGTCCAACAAGGGAC |
| | | TTCCTAGCTCAATCGAAAAGACCATCTCGAAAGCCAAGGGACA |
| | | GCCCCGGGAACCCCAAGTGTATACCCTGCCACCGAGCCAGGAA |
| | | GAAATGACTAAGAACCAAGTCTCATTGACTTGCCTTGTGAAGG |
| | | GCTTCTACCCATCGGATATCGCCGTGGAATGGGAGTCCAACGG |
| | | CCAGCCGGAAAACAACTACAAGACCACCCCTCCGGTGCTGGAC |
| | | TCAGACGGATCCTTCTTCCTCTACTCGCGGCTGACCGTGGATAA |
| | | GAGCAGATGGCAGGAGGGAAATGTGTTCAGCTGTTCTGTGATG |
| | | CATGAAGCCCTGCACAACCACTACACTCAGAAGTCCCTGTCCCT |
| | | CTCCCTGGGA |
| SEQ ID NO: 717 | DNA | CAGGTGCAGCTGGTGCAGTCTGGCGCCGAAGTGAAGAAACCTG |
| | heavy | GCGCCTCCGTGAAGGTGTCCTGCAAGGCCTCTGGCTTCACCCTG |
| | chain | ACCAACTACGGCATGAACTGGGTGCGACAGGCCAGGGGCCAGC |
| | | GGCTGGAATGGATCGGCTGGATCAACACCGACACCGGCGAGCC |
| | | TACCTACGCCGACGACTTCAAGGGCAGATTCGTGTTCTCCCTGG |
| | | ACACCTCCGTGTCCACCGCCTACCTGCAGATCTCCAGCCTGAAG |
| | | GCCGAGGATACCGCCGTGTACTACTGCGCCCGGAACCCCCCTT |
| | | ACTACTACGGCACCAACAACGCCGAGGCCATGGACTATTGGGG |
| | | CCAGGGCACCACCGTGACCGTGTCCTCTGCTTCTACCAAGGGGC |
| | | CCAGCGTGTTCCCCCTGGCCCCCTGCTCCAGAAGCACCAGCGA |
| | | GAGCACAGCCGCCCTGGGCTGCCTGGTGAAGGACTACTTCCCC |
| | | GAGCCCGTGACCGTGTCCTGGAACAGCGGAGCCCTGACCAGCG |
| | | GCGTGCACACCTTCCCCGCCGTGCTGCAGAGCAGCGGCCTGTA |
| | | CAGCCTGAGCAGCGTGGTGACCGTGCCCAGCAGCAGCCTGGGC |
| | | ACCAAGACCTACACCTGTAACGTGGACCACAAGCCCAGCAACA |
| | | CCAAGGTGGACAAGAGGGTGGAGAGCAAGTACGGCCCACCCT |
| | | GCCCCCCCTGCCCAGCCCCCGAGTTCCTGGGCGGACCCAGCGT |
| | | GTTCCTGTTCCCCCCCAAGCCCAAGGACACCCTGATGATCAGCA |
| | | GAACCCCCGAGGTGACCTGTGTGGTGGTGGACGTGTCCCAGGA |
| | | GGACCCCGAGGTCCAGTTCAACTGGTACGTGGACGGCGTGGAG |
| | | GTGCACAACGCCAAGACCAAGCCCAGAGAGGAGCAGTTTAACA |
| | | GCACCTACCGGGTGGTGTCCGTGCTGACCGTGCTGCACCAGGA |
| | | CTGGCTGAACGGCAAAGAGTACAAGTGTAAGGTCTCCAACAAG |
| | | GGCCTGCCAAGCAGCATCGAAAAGACCATCAGCAAGGCCAAG |
| | | GGCCAGCCTAGAGAGCCCCAGGTCTACACCCTGCCACCCAGCC |
| | | AAGAGGAGATGACCAAGAACCAGGTGTCCCTGACCTGTCTGGT |
| | | GAAGGGCTTCTACCCAAGCGACATCGCCGTGGAGTGGGAGAGC |
| | | AACGGCCAGCCCGAGAACAACTACAAGACCACCCCCCCAGTGC |
| | | TGGACAGCGACGGCAGCTTCTTCCTGTACAGCAGGCTGACCGT |
| | | GGACAAGTCCAGATGGCAGGAGGGCAACGFCTTTAGCTGCTCC |
| | | GTGATGCACGAGGCCCTGCACAACCACTACACCCAGAAGAGCC |
| | | TGAGCCTGTCCCTGGGC |
| BAP050-Clone I LC | | |
| SEQ ID NO: 710 | LCDR1 | SSSQDISNYLN |
| (Kabat) | | |
| SEQ ID NO: 711 | LCDR2 | YTSTLHL |
| (Kabat) | | |
| SEQ ID NO: 712 | LCDR3 | QQYYNLPWT |
| (Kabat) | | |
| SEQ ID NO: 713 | LCDR1 | SQDISNY |
| (Chothia) | | |
| SEQ ID NO: 714 | LCDR2 | YTS |
| (Chothia) | | |
| SEQ ID NO: 715 | LCDR3 | YYNLPW |
| (Chothia) | | |
| SEQ ID NO: 718 | VL DNA | D1QMTQSPSSLSASVGDRVTITCSSSQDISNYLNWYLQKP |
| | | GQSPQLLIYYTSTLHLGVPSRFSGSGSGTEFTLTISSLQP |
| | | DDFATYYCQQYYNLPWTFGQGTKVEIK |
| SEQ ID NO: 719 | VL | GGGCGATAGAGTGACTATCACCTGTAGCTCTAGTCAGGATATCT |
| | | CTAACTACCTGAACTGGTATCTGCAGAAGCCCGGTCAATCACCT |
| | | CAGCTGCTGATCTACTACACTAGCACCCTGCACCTGGGCGTGCC |
| | | CTCTAGGTTTAGCGGTAGCGGTAGTGGCACCGAGTTCACCCTGA |
| | | CTATCTCTAGCCTGCAGCCCGACGACTTCGCTACCTACTACTGT |
| | | CAGCAGTACTATAACCTGCCCTGGACCTTCGGTCAAGGCACTA |
| | | AGGTCGAGATTAAG |
| SEQ ID NO: 720 | DNA VL | GACATCCAGATGACCCAGTCCCCCTCCAGCCTGTCTGCTTCCGT |
| | | GGGCGACAGAGTGACCATCACCTGTTCCTCCAGCCAGGACATC |
| | | TCCAACTACCTGAACTGGTATCTGCAGAAGCCCGGCCAGTCCCC |
| | | TCAGCTGCTGATCTACTACACCTCCACCCTGCACCTGGGCGTGC |
| | | CCTCCAGATTTTCCGGCTCTGGCTCTGGCACCGAGTTTACCCTG |
| | | ACCATCAGCTCCCTGCAGCCCGACGACTTCGCCACCTACTACTG |
| | | CCAGCAGTACTACAACCTGCCCTGGACCTTCGGCCAGGGCACC |
| | | AAGGTGGAAATCAAG |
| SEQ ID NO: 721 | Light chain | DIQMTQSPSSLSASVGDRVTITCSSSQDISNLIYYTST |
| | | LHLGVPSRFSGSGSGTEFTLTISSLQPDDFATYYCQQYYNLPW |
| | | TFGQGTKVEIKRTVAAPSVFIFPPSDEQLKSGTASVVCLLNNF |
| | | YPREAKVQWKVDNALQSGNSQESVTEQDSKDSTYSLSSTLTLS |
| | | KADYEKHKVYACEVTHQGLSSPVTKSFNRGEC |
| SEQ ID NO: 722 | DNA light | GGGCGATAGAGTGACTATCACCTGTAGCTCTAGTCAGGATATCT |
| | chain | CTAACTACCTGAACTGGTATCTGCAGAAGCCCGGTCAATCACCT |
| | | CAGCTGCTGATCTACTACACTAGCACCCTGCACCTGGGCGTGCC |
| | | CTCTAGGTTTAGCGGTAGCGGTAGTGGCACCGAGTTCACCCTGA |
| | | CTATCTCTAGCCTGCAGCCCGACGACTTCGCTACCTACTACTGT |
| | | CAGCAGTACTATAACCTGCCCTGGACCTTCGGTCAAGGCACTA |
| | | AGGTCGAGATTAAGCGTACGGTGGCCGCTCCCAGCGTGTTCAT |
| | | CTTCCCCCCCAGCGACGAGCAGCTGAAGAGCGGCACCGCCAGC |
| | | GTGGTGTGCCTGCTGAACAACTTCTACCCCCGGGAGGCCAAGG |
| | | TGCAGTGGAAGGTGGACAACGCCCTGCAGAGCGGCAACAGCCA |
| | | GGAGAGCGTCACCGAGCAGGACAGCAAGGACTCCACCTACAGC |
| | | CTGAGCAGCACCCTGACCCTGAGCAAGGCCGACTACGAGAAGC |
| | | ATAAGGTGTACGCCTGCGAGGTGACCCACCAGGGCCTGTCCAG |
| | | CCCCGTGACCAAGAGCTTCAACAGGGGCGAGTGC |
| SEQ ID NO: 723 | DNA light | GACATCCAGATGACCCAGTCCCCCTCCAGCCTGTCTGCTTCCGT |
| | chain | GGGCGACAGAGTGACCATCACCTGTTCCTCCAGCCAGGACATC |
| | | TCCAACTACCTGAACTGGTATCTGCAGAAGCCCGGCCAGTCCCC |
| | | TCAGCTGCTGATCTACTACACCTCCACCCTGCACCTGGGCGTGC |
| | | CCTCCAGATTTTCCGGCTCTGGCTCTGGCACCGAGTTTACCCTG |
| | | ACCATCAGCTCCCTGCAGCCCGACGACTTCGCCACCTACTACTG |
| | | CCAGCAGTACTACAACCTGCCCTGGACCTTCGGCCAGGGCACC |
| | | AAGGTGGAAATCAAGCGTACGGTGGCCGCTCCCAGCGTGTTCA |
| | | TCTTCCCCCCAAGCGACGAGCAGCTGAAGAGCGGCACCGCCAG |
| | | CGTGGTGTGTCTGCTGAACAACTTCTACCCCAGGGAGGCCAAG |
| | | GTGCAGTGGAAGGTGGACAACGCCCTGCAGAGCGGCAACAGCC |
| | | AGGAGAGCGTCACCGAGCAGGACAGCAAGGACTCCACCTACA |
| | | GCCTGAGCAGCACCCTGACCCTGAGCAAGGCCGACTACGAGAA |
| | | GCACAAGGTGTACGCCTGTGAGGTGACCCACCAGGGCCTGTCC |
| | | AGCCCCGTGACCAAGAGCTTCAACAGGGGCGAGTGC |
| BAP050-Clone J HC | | |
| SEQ ID NO: 701 | HCDR1 | NYGMN |
| (Kabat) | | |
| SEQ ID NO: 702 | HCDR2 | WINTDTGEPTYADDFKG |
| (Kabat) | | |
| SEQ ID NO: 703 | HCDR3 | NPPYYYGTNNAEAMDY |
| (Kabat) | | |
| SEQ ID NO: 704 | HCDR1 | GFTLTNY |
| (Chothia) | | |
| SEQ ID NO: 705 | HCDR2 | NTDTGE |
| (Chothia) | | |
| SEQ ID NO: 703 | HCDR3 | NPPYYYGTNNAEAMDY |
| (Chothia) | | |
| SEQ ID NO: 724 | VH | QVQLVQSGAEVKKPGASVKVSCKASGFTLTNY |
| | | GMNWVRQAPGQGLEWMGWINTDTGEPTYADDF |
| | | KGRFVFSLDTSVSTAYLQISSLKAEDTAVYYC |
| | | ARNPPYYYGTNNAEAMDYWGQGTTVTVSS |
| SEQ ID NO: 725 | DNA VH | CAGGTGCAGCTGGTGCAGTCAGGCGCCGAAGTGAAGAAACCCG |
| | | GCGCTAGTGTGAAAGTCAGCTGTAAAGCTAGTGGCTTCACCCT |
| | | GACTAACTACGGGATGAACTGGGTCCGCCAGGCCCCAGGTCAA |
| | | GGCCTCGAGTGGATGGGCTGGATTAACACCGACACCGGCGAGC |
| | | CTACCTACGCCGACGACTTTAAGGGCAGATTCGTGTTTAGCCTG |
| | | GACACTAGTGTGTCTACCGCCTACCTGCAGATCTCTAGCCTGAA |
| | | GGCCGAGGACACCGCCGTCTACTACTGCGCTAGAAACCCCCCC |
| | | TACTACTACGGCACTAACAACGCCGAGGCTATGGACTACTGGG |
| | | GTCAAGGCACTACCGTGACCGTGTCTAGC |
| SEQ ID NO: 726 | DNA VH | CAGGTGCAGCTGGTGCAGTCTGGCGCCGAAGTGAAGAAACCTG |
| | | GCGCCTCCGTGAAGGTGTCCTGCAAGGCCTCTGGCTTCACCCTG |
| | | ACCAACTACGGCATGAACTGGGTGCGACAGGCCCCTGGACAGG |
| | | GCCTGGAATGGATGGGCTGGATCAACACCGACACCGGCGAGCC |
| | | TACCTACGCCGACGACTTCAAGGGCAGATTCGTGTTCTCCCTGG |
| | | ACACCTCCGTGTCCACCGCCTACCTGCAGATCTCCAGCCTGAAG |
| | | GCCGAGGATACCGCCGTGTACTACTGCGCCCGGAACCCCCCTT |
| | | ACTACTACGGCACCAACAACGCCGAGGCCATGGACTATTGGGG |
| | | CCAGGGCACCACCGTGACCGTGTCCTCT |
| SEQ ID NO: 727 | Heavy | GLEWMGWINTDTGEPTYADDFKGRFVFSLDTSVSTAYLQISSLKA |
| | chain | EDTAVYYCARNPPYYYGTNNAEAMDYWGQGTTVTVSSASTKGP |
| | | SVFPLAPCSRSTSESTAALGCLVKDYFPEPVTVSWNSGALTSGVHT |
| | | FPAVLQSSGLYSLSSVVTVPSSSLGTKTYTCNVDHKPSNTKVDKRV |
| | | ESKYGPPCPPCPAPEFLGGPSVFLFPPKPKDTLMISRTPEVTCVV |
| | | VDVSQEDPEVQFNWYVDGVEVHNAKTKPREEQFNSTYRVVSVLTVL |
| | | HQDWLNGKEYKCKVSNKGLPSSIEKTISKAKGQPREPQVYTLPPS |
| | | QEEMTKNQVSLTCLVKGFYPSDIAVEWESNGQPENNYKTTPPVLD |
| | | SDGSFFLYSRLTVDKSRWQEGNVFSCSVMHEALHNHYTQKSLSLS |
| | | LG |
| SEQ ID NO: 728 | DNA | CAGGTGCAGCTGGTGCAGTCAGGCGCCGAAGTGAAGAAACCCG |
| | heavy | GCGCTAGTGTGAAAGTCAGCTGTAAAGCTAGTGGCTTCACCCT |
| | chain | GACTAACTACGGGATGAACTGGGTCCGCCAGGCCCCAGGTCAA |
| | | GGCCTCGAGTGGATGGGCTGGATTAACACCGACACCGGCGAGC |
| | | CTACCTACGCCGACGACTTTAAGGGCAGATTCGTGTTTAGCCTG |
| | | GACACTAGTGTGTCTACCGCCTACCTGCAGATCTCTAGCCTGAA |
| | | GGCCGAGGACACCGCCGTCTACTACTGCGCTAGAAACCCCCCC |
| | | TACTACTACGGCACTAACAACGCCGAGGCTATGGACTACTGGG |
| | | GTCAAGGCACTACCGTGACCGTGTCTAGCGCTAGCACTAAGGG |
| | | CCCGTCCGTGTTCCCCCTGGCACCTTGTAGCCGGAGCACTAGCG |
| | | AATCCACCGCTGCCCTCGGCTGCCTGGTCAAGGATTACTTCCCG |
| | | GAGCCCGTGACCGTGTCCTGGAACAGCGGAGCCCTGACCTCCG |
| | | GAGTGCACACCTTCCCCGCTGTGCTGCAGAGCTCCGGGCTGTAC |
| | | TCGCTGTCGTCGGTGGTCACGGTGCCTTCATCTAGCCTGGGTAC |
| | | CAAGACCTACACTTGCAACGTGGACCACAAGCCTTCCAACACT |
| | | AAGGTGGACAAGCGCGTCGAATCGAAGTACGGCCCACCGTGCC |
| | | CGCCTTGTCCCGCGCCGGAGTTCCTCGGCGGTCCCTCGGTCTTT |
| | | CTGTTCCCACCGAAGCCCAAGGACACTTTGATGATTTCCCGCAC |
| | | CCCTGAAGTGACATGCGTGGTCGTGGACGTGTCACAGGAAGAT |
| | | CCGGAGGTGCAGTTCAATTGGTACGTGGATGGCGTCGAGGTGC |
| | | ACAACGCCAAAACCAAGCCGAGGGAGGAGCAGTTCAACTCCAC |
| | | TTACCGCGTCGTGTCCGTGCTGACGGTGCTGCATCAGGACTGGC |
| | | TGAACGGGAAGGAGTACAAGTGCAAAGTGTCCAACAAGGGAC |
| | | TTCCTAGCTCAATCGAAAAGACCATCTCGAAAGCCAAGGGACA |
| | | GCCCCGGGAACCCCAAGTGTATACCCTGCCACCGAGCCAGGAA |
| | | GAAATGACTAAGAACCAAGTCTCATTGACTTGCCTTGTGAAGG |
| | | GCTTCTACCCATCGGATATCGCCGTGGAATGGGAGTCCAACGG |
| | | CCAGCCGGAAAACAACTACAAGACCACCCCTCCGGTGCTGGAC |
| | | TCAGACGGATCCTTCTTCCTCTACTCGCGGCTGACCGTGGATAA |
| | | GAGCAGATGGCAGGAGGGAAATGTGTTCAGCTGTTCTGTGATG |
| | | CATGAAGCCCTGCACAACCACTACACTCAGAAGTCCCTGTCCCT |
| | | CTCCCTGGGA |
| SEQ ID NO: 729 | DNA | CAGGTGCAGCTGGTGCAGTCTGGCGCCGAAGTGAAGAAACCTG |
| | Heavy | GCGCCTCCGTGAAGGTGTCCTGCAAGGCCTCTGGCTTCACCCTG |
| | chain | ACCAACTACGGCATGAACTGGGTGCGACAGGCCCCTGGACAGG |
| | | GCCTGGAATGGATGGGCTGGATCAACACCGACACCGGCGAGCC |
| | | TACCTACGCCGACGACTTCAAGGGCAGATTCGTGTTCTCCCTGG |
| | | ACACCTCCGTGTCCACCGCCTACCTGCAGATCTCCAGCCTGAAG |
| | | GCCGAGGATACCGCCGTGTACTACTGCGCCCGGAACCCCCCTT |
| | | ACTACTACGGCACCAACAACGCCGAGGCCATGGACTATTGGGG |
| | | CCAGGGCACCACCGTGACCGTGTCCTCTGCTTCTACCAAGGGGC |
| | | CCAGCGTGTTCCCCCTGGCCCCCTGCTCCAGAAGCACCAGCGA |
| | | GAGCACAGCCGCCCTGGGCTGCCTGGTGAAGGACTACTTCCCC |
| | | GAGCCCGTGACCGTGTCCTGGAACAGCGGAGCCCTGACCAGCG |
| | | GCGTGCACACCTTCCCCGCCGTGCTGCAGAGCAGCGGCCTGTA |
| | | CAGCCTGAGCAGCGTGGTGACCGTGCCCAGCAGCAGCCTGGGC |
| | | ACCAAGACCTACACCTGTAACGTGGACCACAAGCCCAGCAACA |
| | | CCAAGGTGGACAAGAGGGTGGAGAGCAAGTACGGCCCACCCT |
| | | GCCCCCCCTGCCCAGCCCCCGAGTTCCTGGGCGGACCCAGCGT |
| | | GTTCCTGTTCCCCCCCAAGCCCAAGGACACCCTGATGATCAGCA |
| | | GAACCCCCGAGGTGACCTGTGTGGTGGTGGACGTGTCCCAGGA |
| | | GGACCCCGAGGTCCAGTTCAACTGGTACGTGGACGGCGTGGAG |
| | | GTGCACAACGCCAAGACCAAGCCCAGAGAGGAGCAGTTTAACA |
| | | GCACCTACCGGGTGGTGTCCGTGCTGACCGTGCTGCACCAGGA |
| | | CTGGCTGAACGGCAAAGAGTACAAGTGTAAGGTCTCCAACAAG |
| | | GGCCTGCCAAGCAGCATCGAAAAGACCATCAGCAAGGCCAAG |
| | | GGCCAGCCTAGAGAGCCCCAGGTCTACACCCTGCCACCCAGCC |
| | | AAGAGGAGATGACCAAGAACCAGGTGTCCCTGACCTGTCTGGT |
| | | GAAGGGCTTCTACCCAAGCGACATCGCCGTGGAGTGGGAGAGC |
| | | AACGGCCAGCCCGAGAACAACTACAAGACCACCCCCCCAGTGC |
| | | TGGACAGCGACGGCAGCTTCTTCCTGTACAGCAGGCTGACCGT |
| | | GGACAAGTCCAGATGGCAGGAGGGCAACGTCTTTAGCTGCTCC |
| | | GTGATGCACGAGGCCCTGCACAACCACTACACCCAGAAGAGCC |
| | | TGAGCCTGTCCCTGGGC |
| SEQ ID NO: 710 | LCDR1 | SSSQDISNYLN |
| (Kabat) | | |
| SEQ ID NO: 711 | LCDR2 | YTSTLHL |
| (Kabat) | | |
| SEQ ID NO: 712 | LCDR3 | QQYYNLPWT |
| (Kabat) | | |
| SEQ ID NO: 713 | LCDR1 | SQD1SNY |
| (Chothia) | | |
| SEQ ID NO: 714 | LCDR2 | YTS |
| (Chothia) | | |
| SEQ ID NO: 715 | LCDR3 | YYNLPW |
| (Chothia) | | |
| SEQ ID NO: 730 | VL | DIQMTQSPSSLSASVGDRVTITCSSSQDISNYLNWYQQKPGKA |
| | | PKLLIYYTSTLHLGIPPRFSGSGYGTDFTLTINNIESEDAAYY |
| | | FCQQYYNLPWTFGQGTKVEIK |
| SEQ ID NO: 731 | DNA VL | GATATTCAGATGACTCAGTCACCTAGTAGCCTGAGCGCTAGTGT |
| | | GGGCGATAGAGTGACTATCACCTGTAGCTCTAGTCAGGATATCT |
| | | CTAACTACCTGAACTGGTATCAGCAGAAGCCCGGTAAAGCCCC |
| | | TAAGCTGCTGATCTACTACACTAGCACCCTGCACCTGGGAATCC |
| | | CCCCTAGGTTTAGCGGTAGCGGCTACGGCACCGACTTCACCCTG |
| | | ACTATTAACAATATCGAGTCAGAGGACGCCGCCTACTACTTCTG |
| | | TCAGCAGTACTATAACCTGCCCTGGACCTTCGGTCAAGGCACTA |
| | | AGGTCGAGATTAAG |
| SEQ ID NO: 732 | DNA VL | GACATCCAGATGACCCAGTCCCCCTCCAGCCTGTCTGCTTCCGT |
| | | GGGCGACAGAGTGACCATCACCTGTTCCTCCAGCCAGGACATC |
| | | TCCAACTACCTGAACTGGTATCAGCAGAAGCCCGGCAAGGCCC |
| | | CCAAGCTGCTGATCTACTACACCTCCACCCTGCACCTGGGCATC |
| | | CCCCCTAGATTCTCCGGCTCTGGCTACGGCACCGACTTCACCCT |
| | | GACCATCAACAACATCGAGTCCGAGGACGCCGCCTACTACTTC |
| | | TGCCAGCAGTACTACAACCTGCCCTGGACCTTCGGCCAGGGCA |
| | | CCAAGGTGGAAATCAAG |
| SEQ ID NO: 733 | Light | DIQMTQSPSSLSASVGDRVTITCSSSQDISNYLNWYQQKPGKAP |
| | chain | KLLIYYTSTLHLGIPPRFSGSGYGTDFTLTINNIESEDAAYYFC |
| | | QQYYNLPWTFGQGTKVEIKRTVAAPSVFIFPPSDEQLKSGTASV |
| | | VCLLNNFYPREAKVQWKVDNALQSGNSQESVTEQDSKDSTYSLS |
| | | STLTLSKADYEKHKVYACEVTHQGLSSPVTKSFNRGEC |
| SEQ ID NO: 734 | DNA light | GATATTCAGATGACTCAGTCACCTAGTAGCCTGAGCGCTAGTGT |
| | chain | GGGCGATAGAGTGACTATCACCTGTAGCTCTAGTCAGGATATCT |
| | | CTAACTACCTGAACTGGTATCAGCAGAAGCCCGGTAAAGCCCC |
| | | TAAGCTGCTGATCTACTACACTAGCACCCTGCACCTGGGAATCC |
| | | CCCCTAGGTTTAGCGGTAGCGGCTACGGCACCGACTTCACCCTG |
| | | ACTATTAACAATATCGAGTCAGAGGACGCCGCCTACTACTTCTG |
| | | TCAGCAGTACTATAACCTGCCCTGGACCTTCGGTCAAGGCACTA |
| | | AGGTCGAGATTAAGCGTACGGTGGCCGCTCCCAGCGTGTTCAT |
| | | CTTCCCCCCCAGCGACGAGCAGCTGAAGAGCGGCACCGCCAGC |
| | | GTGGTGTGCCTGCTGAACAACTTCTACCCCCGGGAGGCCAAGG |
| | | TGCAGTGGAAGGTGGACAACGCCCTGCAGAGCGGCAACAGCCA |
| | | GGAGAGCGTCACCGAGCAGGACAGCAAGGACTCCACCTACAGC |
| | | CTGAGCAGCACCCTGACCCTGAGCAAGGCCGACTACGAGAAGC |
| | | ATAAGGTGTACGCCTGCGAGGTGACCCACCAGGGCCTGTCCAG |
| | | CCCCGTGACCAAGAGCTTCAACAGGGGCGAGTGC |
| SEQ ID NO: 735 | DNA light | GACATCCAGATGACCCAGTCCCCCTCCAGCCTGTCTGCTTCCGT |
| | chain | GGGCGACAGAGTGACCATCACCTGTTCCTCCAGCCAGGACATC |
| | | TCCAACTACCTGAACTGGTATCAGCAGAAGCCCGGCAAGGCCC |
| | | CCAAGCTGCTGATCTACTACACCTCCACCCTGCACCTGGGCATC |
| | | CCCCCTAGATTCTCCGGCTCTGGCTACGGCACCGACTTCACCCT |
| | | GACCATCAACAACATCGAGTCCGAGGACGCCGCCTACTACTTC |
| | | TGCCAGCAGTACTACAACCTGCCCTGGACCTTCGGCCAGGGCA |
| | | CCAAGGTGGAAATCAAGCGTACGGTGGCCGCTCCCAGCGTGTT |
| | | CATCTTCCCCCCAAGCGACGAGCAGCTGAAGAGCGGCACCGCC |
| | | AGCGTGGTGTGTCTGCTGAACAACTTCTACCCCAGGGAGGCCA |
| | | AGGTGCAGTGGAAGGTGGACAACGCCCTGCAGAGCGGCAACA |
| | | GCCAGGAGAGCGTCACCGAGCAGGACAGCAAGGACTCCACCTA |
| | | CAGCCTGAGCAGCACCCTGACCCTGAGCAAGGCCGACTACGAG |
| | | AAGCACAAGGTGTACGCCTGTGAGGTGACCCACCAGGGCCTGT |
| | | CCAGCCCCGTGACCAAGAGCTTCAACAGGGGCGAGTGC |
| BAP050-Clone IHC | | |
| SEQ ID NO: 736 | HCDR1 | AATTACGGGATGAAC |
| (Kabat) | | |
| | HCDR1 | |
| SEQ ID NO: 737 | | AACTACGGCATGAAC |
| (Kabat) | | |
| SEQ ID NO: 738 | HCDR2 | TGGATTAACACCGACACCGGGGAGCCTACCTACGCGGACGATT |
| (Kabat) | | TCAAGGGA |
| SEQ ID NO: 739 | HCDR2 | TGGATCAACACCGACACCGGCGAGCCTACCTACGCCGACGACT |
| (Kabat) | | TCAAGGGC |
| SEQ ID NO: 740 | HCDR3 | AACCCGCCCTACTACTACGGAACCAACAACGCCGAAGCCATGG |
| (Kabat) | | ACTAC |
| SEQ ID NO: 741 | HCDR3 | AACCCCCCTTACTACTACGGCACCAACAACGCCGAGGCCATGG |
| (Kabat) | | ACTAT |
| SEQ ID NO: 742 | HCDR1 | GGATTCACCCTCACCAATTAC |
| (Chothia) | | |
| SEQ ID NO: 743 | HCDR1 | |
| (Chothia) | | GGCTTCACCCTGACCAACTAC |
| SEQ ID NO: 744 | HCDR2 | AACACCGACACCGGGGAG |
| (Chothia) | | |
| SEQ ID NO: 745 | HCDR2 | AACACCGACACCGGCGAG |
| (Chothia) | | |
| SEQ ID NO: 740 | HCDR3 | AACCCGCCCTACTACTACGGAACCAACAACGCCGAAGCCATGG |
| (Chothia) | | ACTAC |
| SEQ ID NO: 741 | HCDR3 | AACCCCCCTTACTACTACGGCACCAACAACGCCGAGGCCATGG |
| (Chothia) | | ACTAT |
| BAP050-Clone I LC | | |
| SEQ ID NO: 746 | LCDR1 | AGCTCTAGTCAGGATATCTCTAACTACCTGAAC |
| (Kabat) | | |
| SEQ ID NO: 747 | LCDR1 | TCCTCCAGCCAGGACATCTCCAACTACCTGAAC |
| (Kabat) | | |
| SEQ ID NO: 748 | LCDR2 | TACACTAGCACCCTGCACCTG |
| (Kabat) | | |
| SEQ ID NO: 749 | LCDR2 | TACACCTCCACCCTGCACCTG |
| (Kabat) | | |
| SEQ ID NO: 750 | LCDR3 | CAGCAGTACTATAACCTGCCCTGGACC |
| (Kabat) | | |
| SEQ ID NO: 751 | LCDR3 | CAGCAGTACTACAACCTGCCCTGGACC |
| (Kabat) | | |
| SEQ ID NO: 752 | LCDR1 | AGTCAGGATATCTCTAACTAC |
| (Chothia) | | |
| SEQ ID NO: 753 | LCDR1 | AGCCAGGACATCTCCAACTAC |
| (Chothia) | | |
| SEQ ID NO: 754 | LCDR2 | TACACTAGC |
| (Chothia) | | |
| SEQ ID NO: 755 | LCDR2 | TACACCTCC |
| (Chothia) | | |
| SEQ ID NO: 756 | LCDR3 | TACTATAACCTGCCCTGG |
| (Chothia) | | |
| SEQ ID NO: 757 | LCDR3 | TACTACAACCTGCCCTGG |
| (Chothia) | | |
| BAP050-Clone HC | | |
| SEQ ID NO: 758 | HCDR1 | AACTACGGGATGAAC |
| (Kabat) | | |
| SEQ ID NO: 737 | 1HCDR1 | AACTACGGCATGAAC |
| (Kabat) | | |
| SEQ ID NO: 759 | HCDR2 | TGGATTAACACCGACACCGGCGAGCCTACCTACGCCGACGACT |
| (Kabat) | | TTAAGGGC |
| SEQ ID NO: 739 | | |
| (Kabat) | | |
| | HCDR2 | TGGATCAACACCGACACCGGCGAGCCTACCTACGCCGACGACT |
| | | TCAAGGGC |
| SEQ ID NO: 760 | HCDR3 | AACCCCCCCTACTACTACGGCACTAACAACGCCGAGGCTATGG |
| (Kabat) | | ACTAC |
| SEQ ID NO: 741 | HCDR3 | AACCCCCCTTACTACTACGGCACCAACAACGCCGAGGCCATGG |
| (Kabat) | | ACTAT |
| SEQ ID NO: 761 | HCDR1 | GGCTTCACCCTGACTAACTAC |
| (Chothia) | | |
| SEQ ID NO: 743 | HCDR1 | GGCTTCACCCTGACCAACTAC |
| (Chothia) | | |
| SEQ ID NO: 744 | HCDR2 | AACACCGACACCGGGGAG |
| (Chothia) | | |
| SEQ ID NO: 745 | HCDR2 | AACACCGACACCGGCGAG |
| (Chothia) | | |
| SEQ ID NO: 760 | HCDR3 | AACCCCCCCTACTACTACGGCACTAACAACGCCGAGGCTATGG |
| (Chothia) | | ACTAC |
| SEQ ID NO: 741 | HCDR3 | AACCCCCCTTACTACTACGGCACCAACAACGCCGAGGCCATGG |
| (Chothia) | | ACTAT |
| BAP050-Clone J LC | | |
| SEQ ID NO: 746 | LCDR1 | AGCTCTAGTCAGGATATCTCTAACTACCTGAAC |
| (Kabat) | | |
| SEQ ID NO: 747 | LCDR1 | TCCTCCAGCCAGGACATCTCCAACTACCTGAAC |
| (Kabat) | | |
| SEQ ID NO: 748 | LCDR2 | TACACTAGCACCCTGCACCTG |
| (Kabat) | | |
| SEQ ID NO: 749 | LCDR2 | TACACCTCCACCCTGCACCTG |
| (Kabat) | | |
| | LCDR3 | CAGCAGTACTATAACCTGCCCTGGACC |
| SEQ ID NO: 750 | | |
| (Kabat) | | |
| SEQ ID NO: 751 | LCDR3 | CAGCAGTACTACAACCTGCCCTGGACC |
| (Kabat) | | |
| SEQ ID NO: 752 | LCDR1 | AGTCAGGATATCTCTAACTAC |
| (Chothia) | | |
| SEQ ID NO: 753 | LCDR1 | AGCCAGGACATCTCCAACTAC |
| (Chothia) | | |
| SEQ ID NO: 754 | LCDR2 | TACACTACC |
| (Chothia) | | |
| SEQ ID NO: 755 | LCDR2 | TACACCTCC |
| (Chothia) | | |
| SEQ ID NO: 756 | LCDR3 | TACTATAACCTGCCCTGG |
| (Chothia) | | |
| SEQ ID NO: 757 | LCDR3 | TACTACAACCTGCCCTGG |
| (Chothia) | | |

### Other Exemplary LAG-3 Inhibitors

In one embodiment, the anti-LAG-3 antibody molecule is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of BMS-986016, e.g., as disclosed in Table 10.

In one embodiment, the anti-LAG-3 antibody molecule is TSR-033 (Tesaro). In one embodiment, the anti-LAG-3 antibody molecule comprises one or more of the CDR sequences (or collectively all of the CDR sequences), the heavy chain or light chain variable region sequence, or the heavy chain or light chain sequence of TSR-033.

In one embodiment, the anti-LAG-3 antibody molecule is MK-4280 (Merck & Co). In one embodiment, the anti-LAG-3 antibody molecule comprises one or more of the CDR sequences (or collectively all of the CDR sequences), the heavy chain or light chain variable region sequence, or the heavy chain or light chain sequence of MK-4280.

In one embodiment, the anti-LAG-3 antibody molecule is REGN3767 (Regeneron). In one embodiment, the anti-LAG-3 antibody molecule comprises one or more of the CDR sequences (or collectively all of the CDR sequences), the heavy chain or light chain variable region sequence, or the heavy chain or light chain sequence of REGN3767.

In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of IMP731, e.g., as disclosed in Table 10. In one embodiment, the anti-LAG-3 antibody molecule comprises one or more of the CDR sequences (or collectively all of the CDR sequences), the heavy chain or light chain variable region sequence, or the heavy chain or light chain sequence of GSK2831781.

In one embodiment, the anti-LAG-3 antibody molecule is IMP761 (Prima BioMed). In one embodiment, the anti-LAG-3 antibody molecule comprises one or more of the CDR sequences (or collectively all of the CDR sequences), the heavy chain or light chain variable region sequence, or the heavy chain or light chain sequence of IMP761.

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entirety.

In one embodiment, the anti-LAG-3 antibody is an antibody that competes for binding with, and/or binds to the same epitope on LAG-3 as, one of the anti-LAG-3 antibodies described herein. In one embodiment, the anti-LAG-3 inhibitor is a soluble LAG-3 protein, e.g., IMP321 (Prima BioMed), e.g., as disclosed in WO 2009/044273, incorporated by reference in its entirety.

TABLE 10
Amino acid sequences of other exemplary anti-LAG-3 antibody molecules

| | | |
|---|---|---|
| BMS-986016 | | |
| SEQ ID NO: 762 | Heavy chain | QVQLQQWGAGLLKPSETLSLTCAVYGGSFSDYYWNWIRQPPGKGLE |
| | | WIGEINHRGSTNSNPSLKSRVTLSLDTSKNQFSLKLRSVTAADTAVYYC |
| | | AFGYSDYEYNWFDPWGQGTLVTVSSASTKGPSVFPLAPCSRSTSESTA |
| | | ALGCLVKDYFPEPVTVSWNSGALTSGVHTFPAVLQSSGLYSLSSVVTV |
| | | PSSSLGTKTYTCNVDHKPSNTKVDKRVESKYGPPCPPCPAPEFLGGPSV |
| | | FLFPPKPKDTLMISRTPEVTCVVVDVSQEDPEVQFNWYVDGVEVHNA |
| | | KTKPREEQFNSTYRVVSVLTVLHQDWLNGKEYKCKVSNKGLPSSIEKT |
| | | ISKAKGQPREPQVYTLPPSQEEMTKNQVSLTCLVKGFYPSDIAVEWES |
| | | NGQPENNYKTTPPVLDSDGSFFLYSRLTVDKSRWQEGNVFSCSVMHE |
| | | ALHNHYTQKSLSLSLGK |
| SEQ ID NO: 763 | Light chain | EIVLTQSPATLSLSPGERATLSCRASQSISSYLAWYQQKPGQAPRLLIYD |
| | | ASNRATGIPARFSGSGSGTDFTLTISSLEPEDFAVYYCQQRSNWPLTFG |
| | | QGTNLEIKRTVAAPSVFIFPPSDEQLKSGTASVVCLLNNFYPREAKVQW |
| | | KVDNALQSGNSQESVTEQDSKDSTYSLSSTLTLSKADYEKHKVYACE |
| | | VTHQGLSSPVTKSFNRGEC |
| IMP731 | | |
| SEQ ID NO: 764 | Heavy chain | QVQLKESGPGLVAPSQSLSITCTVSGFSLTAYGVNWVRQPPGKGLEWL |
| | | GMIWDDGSTDYNSALKSRLSISKDNSKSQVFLKMNSLQTDDTARYYC |
| | | AREGDVAFDYWGQGTTLTVSSASTKGPSVFPLAPSSKSTSGGTAALGC |
| | | LVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPELLGGPSVF |
| | | LFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEA |
| | | LHNHYTQKSLSLSPGK |
| SEQ ID NO: 765 | Light chain | DIVMTQSPSSLAVSVGQKVTMSCKSSQSLLNGSNQKNYLAWYQQKPG |
| | | QSPKLLVYFASTRDSGVPDRFIGSGSGTDFTLTISSVQAEDLADYFCLQ |
| | | HFGTPPTFGGGTKLEIKRTVAAPSVFIFPPSDEQLKSGTASVVCLLNNFY |
| | | PREAKVQWKVDNALQSGNSQESVTEQDSKDSTYSLSSTLTLSKADYE |
| | | KHKVYACEVTHQGLSSPVTKSFNRGEC |

### Exemplary TIM-3 Inhibitors

In certain embodiments, the anti-CD73 antibody molecule described herein is administered in combination with a TIM-3 inhibitor. The TIM-3 inhibitor may be an antibody, an antigen binding fragment thereof, an immunoadhesin, a fusion protein, or an oligopeptide. In some embodiments, the TIM-3 inhibitor is chosen from MGB453 (Novartis), TSR-022 (Tesaro), or LY3321367 (Eli Lilly).

### Exemplary Anti-TIM-3 Antibody Molecules

In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof,” incorporated by reference in its entirety.

In one embodiment, the anti-TIM-3 antibody molecule comprises at least one, two, three, four, five or six complementarity determining regions (CDRs) (or collectively all of the CDRs) from a heavy and light chain variable region comprising an amino acid sequence shown in Table 11 (e.g., from the heavy and light chain variable region sequences of ABTIM3-hum11 or ABTIM3-hum03 disclosed in Table 11), or encoded by a nucleotide sequence shown in Table 11. In some embodiments, the CDRs are according to the Kabat definition (e.g., as set out in Table 11). In some embodiments, the CDRs are according to the Chothia definition (e.g., as set out in Table 11). In one embodiment, one or more of the CDRs (or collectively all of the CDRs) have one, two, three, four, five, six or more changes, e g, amino acid substitutions (e.g., conservative amino acid substitutions) or deletions, relative to an amino acid sequence shown in Table 11, or encoded by a nucleotide sequence shown in Table 11.

In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 802, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 11. In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 820, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 11.

In one embodiment, the anti-TIM-3 antibody molecule comprises a VH comprising the amino acid sequence of SEQ ID NO: 806, or an amino acid sequence having at least about 85%, 90%, 95%, or 99% sequence identity to SEQ ID NO: 806. In one embodiment, the anti-TIM-3 antibody molecule comprises a VL comprising the amino acid sequence of SEQ ID NO: 816, or an amino acid sequence having at least about 85%, 90%, 95%, or 99% sequence identity to SEQ ID NO: 816. In one embodiment, the anti-TIM-3 antibody molecule comprises a VH comprising the amino acid sequence of SEQ ID NO: 822, or an amino acid sequence having at least about 85%, 90%, 95%, or 99% sequence identity to SEQ ID NO: 822. In one embodiment, the anti-TIM-3 antibody molecule comprises a VL comprising the amino acid sequence of SEQ ID NO: 826, or an amino acid sequence having at least about 85%, 90%, 95%, or 99% sequence identity to SEQ ID NO: 826. In one embodiment, the anti-TIM-3 antibody molecule comprises a VH comprising the amino acid sequence of SEQ ID NO: 806 and a VL comprising the amino acid sequence of SEQ ID NO: 816. In one embodiment, the anti-TIM-3 antibody molecule comprises a VH comprising the amino acid sequence of SEQ ID NO: 822 and a VL comprising the amino acid sequence of SEQ ID NO: 826.

In one embodiment, the antibody molecule comprises a VH encoded by the nucleotide sequence of SEQ ID NO: 807, or a nucleotide sequence having at least about 85%, 90%, 95%, or 99% sequence identity to SEQ ID NO: 807. In one embodiment, the antibody molecule comprises a VL encoded by the nucleotide sequence of SEQ ID NO: 817, or a nucleotide sequence having at least about 85%, 90%, 95%, or 99% sequence identity to SEQ ID NO: 817. In one embodiment, the antibody molecule comprises a VH encoded by the nucleotide sequence of SEQ ID NO: 823, or a nucleotide sequence having at least about 85%, 90%, 95%, or 99% sequence identity to SEQ ID NO: 823. In one embodiment, the antibody molecule comprises a VL encoded by the nucleotide sequence of SEQ ID NO: 827, or a nucleotide sequence having at least about 85%, 90%, 95%, or 99% sequence identity to SEQ ID NO: 827. In one embodiment, the antibody molecule comprises a VH encoded by the nucleotide sequence of SEQ ID NO: 807 and a VL encoded by the nucleotide sequence of SEQ ID NO: 817. In one embodiment, the antibody molecule comprises a VH encoded by the nucleotide sequence of SEQ ID NO: 823 and a VL encoded by the nucleotide sequence of SEQ ID NO: 827.

In one embodiment, the anti-TIM-3 antibody molecule comprises a heavy chain comprising the amino acid sequence of SEQ ID NO: 808, or an amino acid sequence having at least about 85%, 90%, 95%, or 99% sequence identity to SEQ ID NO: 808. In one embodiment, the anti-TIM-3 antibody molecule comprises a light chain comprising the amino acid sequence of SEQ ID NO: 818, or an amino acid sequence having at least about 85%, 90%, 95%, or 99% sequence identity to SEQ ID NO: 818. In one embodiment, the anti-TIM-3 antibody molecule comprises a heavy chain comprising the amino acid sequence of SEQ ID NO: 824, or an amino acid sequence having at least about 85%, 90%, 95%, or 99% sequence identity to SEQ ID NO: 824. In one embodiment, the anti-TIM-3 antibody molecule comprises a light chain comprising the amino acid sequence of SEQ ID NO: 828, or an amino acid sequence having at least about 85%, 90%, 95%, or 99% sequence identity to SEQ ID NO: 828. In one embodiment, the anti-TIM-3 antibody molecule comprises a heavy chain comprising the amino acid sequence of SEQ ID NO: 808 and a light chain comprising the amino acid sequence of SEQ ID NO: 818. In one embodiment, the anti-TIM-3 antibody molecule comprises a heavy chain comprising the amino acid sequence of SEQ ID NO: 824 and a light chain comprising the amino acid sequence of SEQ ID NO: 828.

In one embodiment, the antibody molecule comprises a heavy chain encoded by the nucleotide sequence of SEQ ID NO: 809, or a nucleotide sequence having at least about 85%, 90%, 95%, or 99% sequence identity to SEQ ID NO: 809. In one embodiment, the antibody molecule comprises a light chain encoded by the nucleotide sequence of SEQ ID NO: 819, or a nucleotide sequence having at least about 85%, 90%, 95%, or 99% sequence identity to SEQ ID NO: 819. In one embodiment, the antibody molecule comprises a heavy chain encoded by the nucleotide sequence of SEQ ID NO: 825, or a nucleotide sequence having at least about 85%, 90%, 95%, or 99% sequence identity to SEQ ID NO: 825. In one embodiment, the antibody molecule comprises a light chain encoded by the nucleotide sequence of SEQ ID NO: 829, or a nucleotide sequence having at least about 85%, 90%, 95%, or 99% sequence identity to SEQ ID NO: 829. In one embodiment, the antibody molecule comprises a heavy chain encoded by the nucleotide sequence of SEQ ID NO: 809 and a light chain encoded by the nucleotide sequence of SEQ ID NO: 819. In one embodiment, the antibody molecule comprises a heavy chain encoded by the nucleotide sequence of SEQ ID NO: 825 and a light chain encoded by the nucleotide sequence of SEQ ID NO: 829.

The antibody molecules described herein can be made by vectors, host cells, and methods described in US 2015/0218274, incorporated by reference in its entirety.

TABLE 11
Amino acid and nucleotide sequences
of exemplary anti-TIM-3 antibody molecules

| | | |
|---|---|---|
| ABTIM3-hum11 | | |
| SEQ ID NO: 801 (Kabat) | HCDR1 | SYNMH |
| SEQ ID NO: 802 (Kabat) | HCDR2 | DIYPGNGDTSYNQKFKG |
| SEQ ID NO: 803 (Kabat) | HCDR3 | VGGAFPMDY |
| SEQ ID NO: 804 (Chothia) | HCDR1 | GYTFTSY |
| SEQ ID NO: 805 (Chothia) | HCDR2 | YPGNGD |
| SEQ ID NO: 803 (Chothia) | HCDR3 | VGGAFPMDY |
| SEQ ID NO: 806 | VH | QVQLVQSGAEVKKPGSSVKVSCKASGYTFTSYNMHWVRQAPG |
| | | QGLEWMGDIYPGNGDTSYNQKFKGRVTITADKSTSTVYMELSS |
| | | LRSEDTAVYYCARVGGAFPMDYWGQGTTVTVSS |
| SEQ ID NO: 807 | DNA VH | CAGGTGCAGCTGGTGCAGTCAGGCGCCGAAGTGAAGAAACC |
| | | CGGCTCTAGCGTGAAAGTTTCTTGTAAAGCTAGTGGCTACAC |
| | | CTTCACTAGCTATAATATGCACTGGGTTCGCCAGGCCCCAGG |
| | | GCAAGGCCTCGAGTGGATGGGCGATATCTACCCCGGGAACGG |
| | | CGACACTAGTTATAATCAGAAGTTTAAGGGTAGAGTCACTAT |
| | | CACCGCCGATAAGTCTACTAGCACCGTCTATATGGAACTGAG |
| | | TTCCCTGAGGTCTGAGGACACCGCCGTCTACTACTGCGCTAG |
| | | AGTGGGCGGAGCCTTCCCTATGGACTACTGGGGTCAAGGCAC |
| | | TACCGTGACCGTGTCTAGC |
| SEQ ID NO: 808 | Heavy | QVQLVQSGAEVKKPGSSVKVSCKASGYTFTSYNMHWVRQAPG |
| | chain | QGLEWMGDIYPGNGDTSYNQKFKGRVTITADKSTSTVYMELSS |
| | | LRSEDTAVYYCARVGGAFPMDYWGQGTTVTVSSASTKGPSVFP |
| | | LAPCSRSTSESTAALGCLVKDYFPEPVTVSWNSGALTSGVHTFP |
| | | AVLQSSGLYSLSSVVTVPSSSLGTKTYTCNVDHKPSNTKVDKRV |
| | | ESKYGPPCPPCPAPEFLGGPSVFLFPPKPKDTLMISRTPEVTCVVV |
| | | DVSQEDPEVQFNWYVDGVEVHNAKTKPREEQFNSTYRVVSVLT |
| | | VLHQDWLNGKEYKCKVSNKGLPSSIEKTISKAKGQPREPQVYTL |
| | | PPSQEEMTKNQVSLTCLVKGFYPSDIAVEWESNGQPENNYKTTP |
| | | PVLDSDGSFFLYSRLTVDKSRWQEGNVFSCSVMHEALHNHYTQ |
| | | KSLSLSLG |
| SEQ ID NO: 809 | DNA | CAGGTGCAGCTGGTGCAGTCAGGCGCCGAAGTGAAGAAACC |
| | heavy | CGGCTCTAGCGTGAAAGTTTCTTGTAAAGCTAGTGGCTACAC |
| | chain | CTTCACTAGCTATAATATGCACTGGGTTCGCCAGGCCCCAGG |
| | | GCAAGGCCTCGAGTGGATGGGCGATATCTACCCCGGGAACGG |
| | | CGACACTAGTTATAATCAGAAGTTTAAGGGTAGAGTCACTAT |
| | | CACCGCCGATAAGTCTACTAGCACCGTCTATATGGAACTGAG |
| | | TTCCCTGAGGTCTGAGGACACCGCCGTCTACTACTGCGCTAG |
| | | AGTGGGCGGAGCCTTCCCTATGGACTACTGGGGTCAAGGCAC |
| | | TACCGTGACCGTGTCTAGCGCTAGCACTAAGGGCCCGTCCGT |
| | | GTTCCCCCTGGCACCTTGTAGCCGGAGCACTAGCGAATCCAC |
| | | CGCTGCCCTCGGCTGCCTGGTCAAGGATTACTTCCCGGAGCC |
| | | CGTGACCGTGTCCTGGAACAGCGGAGCCCTGACCTCCGGAGT |
| | | GCACACCTTCCCCGCTGTGCTGCAGAGCTCCGGGCTGTACTC |
| | | GCTGTCGTCGGTGGTCACGGTGCCTTCATCTAGCCTGGGTACC |
| | | AAGACCTACACTTGCAACGTGGACCACAAGCCTTCCAACACT |
| | | AAGGTGGACAAGCGCGTCGAATCGAAGTACGGCCCACCGTG |
| | | CCCGCCTTGTCCCGCGCCGGAGTTCCTCGGCGGTCCCTCGGTC |
| | | TTTCTGTTCCCACCGAAGCCCAAGGACACTTTGATGATTTCCC |
| | | GCACCCCTGAAGTGACATGCGTGGTCGTGGACGTGTCACAGG |
| | | AAGATCCGGAGGTGCAGTTCAATTGGTACGTGGATGGCGTCG |
| | | AGGTGCACAACGCCAAAACCAAGCCGAGGGAGGAGCAGTTC |
| | | AACTCCACTTACCGCGTCGTGTCCGTGCTGACGGTGCTGCATC |
| | | AGGACTGGCTGAACGGGAAGGAGTACAAGTGCAAAGTGTCC |
| | | AACAAGGGACTTCCTAGCTCAATCGAAAAGACCATCTCGAAA |
| | | GCCAAGGGACAGCCCCGGGAACCCCAAGTGTATACCCTGCCA |
| | | CCGAGCCAGGAAGAAATGACTAAGAACCAAGTCTCATTGACT |
| | | TGCCTTGTGAAGGGCTTCTACCCATCGGATATCGCCGTGGAA |
| | | TGGGAGTCCAACGGCCAGCCGGAAAACAACTACAAGACCAC |
| | | CCCTCCGGTGCTGGACTCAGACGGATCCTTCTTCCTCTACTCG |
| | | CGGCTGACCGTGGATAAGAGCAGATGGCAGGAGGGAAATGT |
| | | GTTCAGCTGTTCTGTGATGCATGAAGCCCTGCACAACCACTA |
| | | CACTCAGAAGTCCCTGTCCCTCTCCCTGGGA |
| SEQ ID NO: 810 (Kabat) | LCDR1 | RASESVEYYGTSLMQ |
| SEQ ID NO: 811 (Kabat) | LCDR2 | AASNVES |
| SEQ ID NO: 812 (Kabat) | LCDR3 | QQSRKDPST |
| SEQ ID NO: 813 (Chothia) | LCDR1 | SESVEYYGTSL |
| SEQ ID NO: 814 (Chothia) | LCDR2 | AAS |
| SEQ ID NO: 815 (Chothia) | LCDR3 | SRKDPS |
| SEQ ID NO: 816 | VL | AIQLTQSPSSLSASVGDRVTITCRASESVEYYGTSLMQYQQKP |
| | | GKAPKLLIYAASNVESGVPSRFSGSGSGTDFTLTISSLQPEDFATY |
| | | FCQQSRKDPSTFGGGTKVEIK |
| SEQ ID NO: 817 | DNA VL | GCTATTCAGCTGACTCAGTCACCTAGTAGCCTGAGCGCTAGT |
| | | GTGGGCGATAGAGTGACTATCACCTGTAGAGCTAGTGAATCA |
| | | GTCGAGTACTACGGCACTAGCCTGATGCAGTGGTATCAGCAG |
| | | AAGCCCGGGAAAGCCCCTAAGCTGCTGATCTACGCCGCCTCT |
| | | AACGTGGAATCAGGCGTGCCCTCTAGGTTTAGCGGTAGCGGT |
| | | AGTGGCACCGACTTCACCCTGACTATCTCTAGCCTGCAGCCC |
| | | GAGGACTTCGCTACCTACTTCTGTCAGCAGTCTAGGAAGGAC |
| | | CCTAGCACCTTCGGCGGAGGCACTAAGGTCGAGATTAAG |
| SEQ ID NO: 818 | Light | AIQLTQSPSSLSASVGDRVTITCRASESVEYYGTSLMQWYQQKP |
| | chain | GKAPKLLIYAASNVESGVPSRFSGSGSGTDFTLTISSLQPEDFATY |
| | | FCQQSRKDPSTFGGGTKVEIKRTVAAPSVFIFPPSDEQLKSGTAS |
| | | VVCLLNNFYPREAKVQWKVDNALQSGNSQESVTEQDSKDSTYS |
| | | LSSTLTLSKADYEKHKVYACEVTNGLSSPVTKSFNRGEC |
| SEQ ID NO: 819 | DNA light | GCTATTAGCTGACTCAGTCACCTAGTAGCCTGAGCGCTAGT |
| | chain | GTGGGCGATAGAGTGACTATCACCTGTAGAGCTAGTGAATCA |
| | | GTCGAGTACTACGGCACTAGCCTGATGCAGTGGTATCAGCAG |
| | | AAGCCCGGGAAAGCCCCTAAGCTGCTGATCTACGCCGCCTCT |
| | | AACGTGGAATCAGGCGTGCCCTCTAGGTTTAGCGGTAGCGGT |
| | | AGTGGCACCGACTTCACCCTGACTATCTCTAGCCTGCAGCCC |
| | | GAGGACTTCGCTACCTACTTCTGTCAGCAGTCTAGGAAGGAC |
| | | CCTAGCACCTTCGGCGGAGGCACTAAGGTCGAGATTAAGCGT |
| | | ACGGTGGCCGCTCCCAGCGTGTTCATCTTCCCCCCCAGCGAC |
| | | GAGCAGCTGAAGAGCGGCACCGCCAGCGTGGTGTGCCTGCTG |
| | | AACAACTTCTACCCCCGGGAGGCCAAGGTGCAGTGGAAGGTG |
| | | GACAACGCCCTGCAGAGCGGCAACAGCCAGGAGAGCGTCAC |
| | | CGAGCAGGACAGCAAGGACTCCACCTACAGCCTGAGCAGCA |
| | | CCCTGACCCTGAGCAAGGCCGACTACGAGAAGCATAAGGTGT |
| | | ACGCCTGCGAGGTGACCCACCAGGGCCTGTCCAGCCCCGTGA |
| | | CCAAGAGCTTCAACAGGGGCGAGTGC |
| ABTIM3-hum03 | | |
| SEQ ID NO: 801 (Kabat) | HCDR1 | SYNMH |
| SEQ ID NO: 820 (Kabat) | HCDR2 | DIYPGQGDTSYNQKFKG |
| SEQ ID NO: 803 (Kabat) | HCDR3 | VGGAFPMDY |
| SEQ ID NO: 804 (Chothia) | HCDR1 | GYTFTSY |
| SEQ ID NO: 821 (Chothia) | HCDR2 | YPGQGD |
| SEQ ID NO: 803 (Chothia) | HCDR3 | VGGAFPMDY |
| SEQ ID NO: 822 | VH | QVQLVQSGAEVKKPGASVKVSCKASGYTFTSYNMHWVRQAPG |
| | | QGLEWIGDIYPGQGDTSYNQKFKGRATMTADKSTSTVYMELSS |
| | | LRSEDTAVYYCARVGGAFPMDYWGQGTLVTVSS |
| SEQ ID NO: 823 | DNA VH | CAGGTGCAGCTGGTGCAGTCAGGCGCCGAAGTGAAGAAACC |
| | | CGGCGCTAGTGTGAAAGTTAGCTGTAAAGCTAGTGGCTATAC |
| | | TTTCACTTCTTATAATATGCACTGGGTCCGCCAGGCCCCAGGT |
| | | CAAGGCCTCGAGTGGATCGGCGATATCTACCCCGGTCAAGGC |
| | | GACACTTCCTATAATCAGAAGTTTAAGGGTAGAGCTACTATG |
| | | ACCGCCGATAAGTCTACTTCTACCGTCTATATGGAACTGAGTT |
| | | CCCTGAGGTCTGAGGACACCGCCGTCTACTACTGCGCTAGAG |
| | | TGGGCGGAGCCTTCCCAATGGACTACTGGGGTCAAGGCACCC |
| | | TGGTCACCGTGTCTAGC |
| SEQ ID NO: 824 | Heavy | QVQLVQSGAEVKKPGASVKVSCKASGYTFTSYNMHWVRQAPG |
| | chain | QGLEWIGDIYPGQGDTSYNQKFKGRATMTADKSTSTVYMELSS |
| | | LRSEDTAVYYCARVGGAFPMDYWGQGTLVTVSSASTKGPSVFP |
| | | LAPCSRSTSESTAALGCLVKDYFPEPVTVSWNSGALTSGVHTFP |
| | | AVLQSSGLYSLSSVVTVPSSSLGTKTYTCNVDHKPSNTKVDKRV |
| | | ESKYGPPCPPCPAPEFLGGPSVFLFPPKPKDTLMISRTPEVTCVVV |
| | | DVSQEDPEVQFNWYVDGVEVHNAKTKPREEQFNSTYRVVSVLT |
| | | VLHQDWLNGKEYKCKVSNKGLPSSIEKTISKAKGQPREPQVYTL |
| | | PPSQEEMTKNQVSLTCLVKGFYPSDIAVEWESNGQPENNYKTTP |
| | | PVLDSDGSFFLYSRLTVDKSRWQEGNVFSCSVMHEALHNHYTQ |
| | | KSLSLSLG |
| SEQ ID NO: 825 | DNA | CAGGTGCAGCTGGTGCAGTCAGGCGCCGAAGTGAAGAAACC |
| | heavy | CGGCGCTAGTGTGAAAGTTAGCTGTAAAGCTAGTGGCTATAC |
| | chain | TTTCACTTCTTATAATATGCACTGGGTCCGCCAGGCCCCAGGT |
| | | CAAGGCCTCGAGTGGATCGGCGATATCTACCCCGGTCAAGGC |
| | | GACACTTCCTATAATCAGAAGTTTAAGGGTAGAGCTACTATG |
| | | ACCGCCGATAAGTCTACTTCTACCGTCTATATGGAACTGAGTT |
| | | CCCTGAGGTCTGAGGACACCGCCGTCTACTACTGCGCTAGAG |
| | | TGGGCGGAGCCTTCCCAATGGACTACTGGGGTCAAGGCACCC |
| | | TGGTCACCGTGTCTAGCGCTAGCACTAAGGGCCCGTCCGTGT |
| | | TCCCCCTGGCACCTTGTAGCCGGAGCACTAGCGAATCCACCG |
| | | CTGCCCTCGGCTGCCTGGTCAAGGATTACTTCCCGGAGCCCGT |
| | | GACCGTGTCCTGGAACAGCGGAGCCCTGACCTCCGGAGTGCA |
| | | CACCTTCCCCGCTGTGCTGCAGAGCTCCGGGCTGTACTCGCTG |
| | | TCGTCGGTGGTCACGGTGCCTTCATCTAGCCTGGGTACCAAG |
| | | ACCTACACTTGCAACGTGGACCACAAGCCTTCCAACACTAAG |
| | | GTGGACAAGCGCGTCGAATCGAAGTACGGCCCACCGTGCCCG |
| | | CCTTGTCCCGCGCCGGAGTTCCTCGGCGGTCCCTCGGTCTTTC |
| | | TGTTCCCACCGAAGCCCAAGGACACTTTGATGATTTCCCGCA |
| | | CCCCTGAAGTGACATGCGTGGTCGTGGACGTGTCACAGGAAG |
| | | ATCCGGAGGTGCAGTTCAATTGGTACGTGGATGGCGTCGAGG |
| | | TGCACAACGCCAAAACCAAGCCGAGGGAGGAGCAGTTCAAC |
| | | TCCACTTACCGCGTCGTGTCCGTGCTGACGGTGCTGCATCAGG |
| | | ACTGGCTGAACGGGAAGGAGTACAAGTGCAAAGTGTCCAAC |
| | | AAGGGACTTCCTAGCTCAATCGAAAAGACCATCTCGAAAGCC |
| | | AAGGGACAGCCCCGGGAACCCCAAGTGTATACCCTGCCACCG |
| | | AGCCAGGAAGAAATGACTAAGAACCAAGTCTCATTGACTTGC |
| | | CTTGTGAAGGGCTTCTACCCATCGGATATCGCCGTGGAATGG |
| | | GAGTCCAACGGCCAGCCGGAAAACAACTACAAGACCACCCC |
| | | TCCGGTGCTGGACTCAGACGGATCCTTCTTCCTCTACTCGCGG |
| | | CTGACCGTGGATAAGAGCAGATGGCAGGAGGGAAATGTGTT |
| | | CAGCTGTTCTGTGATGCATGAAGCCCTGCACAACCACTACAC |
| | | TCAGAAGTCCCTGTCCCTCTCCCTGGGA |
| SEQ ID NO: 810 (Kabat) | LCDR1 | RASESVEYYGTSLMQ |
| SEQ ID NO: 811 (Kabat) | LCDR2 | AASNVES |
| SEQ ID NO: 812 (Kabat) | LCDR3 | QQSRKDPST |
| SEQ ID NO: 813 (Chothia) | LCDR1 | SESVEYYGTSL |
| SEQ ID NO: 814 (Chothia) | LCDR2 | AAS |
| SEQ ID NO: 815 (Chothia) | LCDR3 | SRKDPS |
| SEQ ID NO: 826 | VL | DIVLTQSPDSLAVSLGERATINCRASESVEYYGTSLMQWYQQKP |
| | | GQPPKLLIYAASNVESGVPDRFSGSGSGTDFTLTISSLQAEDVAV |
| | | YYCQQSRKDPSTFGGGTKVEIK |
| SEQ ID NO: 827 | DNA VL | GATATCGTCCTGACTCAGTCACCCGATAGCCTGGCCGTCAGC |
| | | CTGGGCGAGCGGGCTACTATTAACTGTAGAGCTAGTGAATCA |
| | | GTCGAGTACTACGGCACTAGCCTGATGCAGTGGTATCAGCAG |
| | | AAGCCCGGTCAACCCCCTAAGCTGCTGATCTACGCCGCCTCT |
| | | AACGTGGAATCAGGCGTGCCCGATAGGTTTAGCGGTAGCGGT |
| | | AGTGGCACCGACTTCACCCTGACTATTAGTAGCCTGCAGGCC |
| | | GAGGACGTGGCCGTCTACTACTGTCAGCAGTCTAGGAAGGAC |
| | | CCTAGCACCTTCGGCGGAGGCACTAAGGTCGAGATTAAG |
| SEQ ID NO: 828 | Light | DIVLTQSPDSLAVSLGERATINCRASESVEYYGTSLMQWYQQKP |
| | chain | GQPPKLLIYAASNVESGVPDRFSGSGSGTDFTLTISSLQAEDVAV |
| | | YYCQQSRKDPSTFGGGTKVEIKRTVAAPSVFIFPPSDEQLKSGTA |
| | | SVVCLLNNFYPREAKVQWKVDNALQSGNSQESVTEQDSKDSTY |
| | | SLSSTLTLSKADYEKHKVYACEVTHQGLSSPVTKSFNRGEC |
| SEQ ID NO: 829 | DNA light | GATATCGTCCTGACTCAGTCACCCGATAGCCTGGCCGTCAGC |
| | chain | CTGGGCGAGCGGGCTACTATTAACTGTAGAGCTAGTGAATCA |
| | | GTCGAGTACTACGGCACTAGCCTGATGCAGTGGTATCAGCAG |
| | | AAGCCCGGTCAACCCCCTAAGCTGCTGATCTACGCCGCCTCT |
| | | AACGTGGAATCAGGCGTGCCCGATAGGTTTAGCGGTAGCGGT |
| | | AGTGGCACCGACTTCACCCTGACTATTAGTAGCCTGCAGGCC |
| | | GAGGACGTGGCCGTCTACTACTGTCAGCAGTCTAGGAAGGAC |
| | | CCTAGCACCTTCGGCGGAGGCACTAAGGTCGAGATTAAGCGT |
| | | ACGGTGGCCGCTCCCAGCGTGTTCATCTTCCCCCCCAGCGAC |
| | | GAGCAGCTGAAGAGCGGCACCGCCAGCGTGGTGTGCCTGCTG |
| | | AACAACTTCTACCCCCGGGAGGCCAAGGTGCAGTGGAAGGTG |
| | | GACAACGCCCTGCAGAGCGGCAACAGCCAGGAGAGCGTCAC |
| | | CGAGCAGGACAGCAAGGACTCCACCTACAGCCTGAGCAGCA |
| | | CCCTGACCCTGAGCAAGGCCGACTACGAGAAGCATAAGGTGT |
| | | ACGCCTGCGAGGTGACCCACCAGGGCCTGTCCAGCCCCGTGA |
| | | CCAAGAGCTTCAACAGGGGCGAGTGC |

### Other Exemplary TIM-3 Inhibitors

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of TSR-022. In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121, e.g., as disclosed in Table 12. APE5137, APE5121, and other anti-TIM-3 antibodies are disclosed in WO 2016/161270, incorporated by reference in its entirety.

In one embodiment, the anti-TIM-3 antibody molecule is LY3321367 (Eli Lilly). In one embodiment, the anti-TIM-3 antibody molecule comprises one or more of the CDR sequences (or collectively all of the CDR sequences), the heavy chain or light chain variable region sequence, or the heavy chain or light chain sequence of LY3321367.

In one embodiment, the anti-TIM-3 antibody molecule is the antibody clone F38-2E2. In one embodiment, the anti-TIM-3 antibody molecule comprises one or more of the CDR sequences (or collectively all of the CDR sequences), the heavy chain or light chain variable region sequence, or the heavy chain or light chain sequence of F38-2E2.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In one embodiment, the anti-TIM-3 antibody is an antibody that competes for binding with, and/or binds to the same epitope on TIM-3 as, one of the anti-TIM-3 antibodies described herein.

TABLE 12
Amino acid sequences of other exemplary anti-TIM-3 antibody molecules

| | | |
|---|---|---|
| APE5137 | | |
| SEQ ID NO: 830 | VH | EVQLLESGGGLVQPGGSLRLSCAAASGFTFSSYDMSWVRQAPGKGLDWVS |
| | | TISGGGTYTYYQDSVKGRFTISRDNSKNTLYLQMNSLRAEDTAVYYCASMD |
| | | YWGQGTTVTVSSA |
| SEQ ID NO: 831 | VL | DIQMTQSPSSLSASVGDRVTITCRASQSIRRYLNWYHQKPGKAPKLLIYGAS |
| | | TLQSGVPSRFSGSGSGTDFTLTISSLQPEDFAVYYCQQSHSAPLTFGGGTKVE |
| | | IKR |
| APE5121 | | |
| SEQ ID NO: 832 | VH | EVQVLESGGGLVQPGGSLRLYCVASGFTFSGSYAMSWVRQAPGKGLEWVS |
| | | AISGSGGSTYYADSVKGRFTISRDNSKNTLYLQMNSLRAEDTAVYYCAKKY |
| | | YVGPADYWGQGTLVTVSSG |
| SEQ ID NO: 833 | VL | DIVMTQSPDSLAVSLGERATINCKSSQSVLYSSNNKNYLAWYQHKPGQPPK |
| | | LLIYWASTRESGVPDRFSGSGSGTDFTLTISSLQAEDVAVYYCQQYYSSPLTF |
| | | GGGTKIEVK |

### Exemplary CTLA-4 Inhibitors

In certain embodiments, the anti-CD73 antibody molecule described herein is administered in combination with a CTLA-4 inhibitor. The CTLA-4 inhibitor may be an antibody, an antigen binding fragment thereof, an immunoadhesin, a fusion protein, or an oligopeptide. In some embodiments, the CTLA-4 inhibitor is Ipilimumab (Yervoy®, Bristol-Myers Squibb) or Tremelimumab (Pfizer). The antibody Ipilimumab and other anti-CTLA-4 antibodies are disclosed in U.S. Pat. No. 6,984,720, herein incorporated by reference. The antibody Tremelimumab and other anti-CTLA-4 antibodies are disclosed in U.S. Pat. No. 7,411,057, herein incorporated by reference.

### Exemplary GITR Agonists

In certain embodiments, the anti-CD73 antibody molecule described herein is administered in combination with a GITR agonist. The GITR agonist may be an antibody, an antigen binding fragment thereof, an immunoadhesin, a fusion protein, or an oligopeptide. In some embodiments, the GITR agonist is GWN323 (Novartis), BMS-986156 (BMS), MK-4166 or MK-1248 (Merck), TRX518 (Leap Therapeutics), INCAGN1876 (Incyte/Agenus), AMG 228 (Amgen), or INBRX-110 (Inhibrx).

### Exemplary Anti-GITR Antibody Molecules

In one embodiment, the GITR agonist is an anti-GITR antibody molecule. In one embodiment, the GITR agonist is an anti-GITR antibody molecule as described in WO 2016/057846, published on Apr. 14, 2016, entitled “Compositions and Methods of Use for Augmented Immune Response and Cancer Therapy,” incorporated by reference in its entirety.

In one embodiment, the anti-GITR antibody molecule comprises at least one, two, three, four, five or six complementarity determining regions (CDRs) (or collectively all of the CDRs) from a heavy and light chain variable region comprising an amino acid sequence shown in Table 13 (e.g., from the heavy and light chain variable region sequences of MAB7 disclosed in Table 13), or encoded by a nucleotide sequence shown in Table 13. In some embodiments, the CDRs are according to the Kabat definition (e.g., as set out in Table 13). In some embodiments, the CDRs are according to the Chothia definition (e.g., as set out in Table 13). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 13, or encoded by a nucleotide sequence shown in Table 13.

In one embodiment, the anti-GITR antibody molecule comprises a heavy chain variable region (VH) comprising a VHCDR1 amino acid sequence of SEQ ID NO: 909, a VHCDR2 amino acid sequence of SEQ ID NO: 911, and a VHCDR3 amino acid sequence of SEQ ID NO: 913; and a light chain variable region (VL) comprising a VLCDR1 amino acid sequence of SEQ ID NO: 914, a VLCDR2 amino acid sequence of SEQ ID NO: 916, and a VLCDR3 amino acid sequence of SEQ ID NO: 918, each disclosed in Table 13.

In one embodiment, the anti-GITR antibody molecule comprises a VH comprising the amino acid sequence of SEQ ID NO: 901, or an amino acid sequence at least 85%, 90%, 95%, or 99% identical or higher to SEQ ID NO: 901. In one embodiment, the anti-GITR antibody molecule comprises a VL comprising the amino acid sequence of SEQ ID NO: 902, or an amino acid sequence at least 85%, 90%, 95%, or 99% identical or higher to SEQ ID NO: 902. In one embodiment, the anti-GITR antibody molecule comprises a VH comprising the amino acid sequence of SEQ ID NO: 901 and a VL comprising the amino acid sequence of SEQ ID NO: 902.

In one embodiment, the antibody molecule comprises a VH encoded by the nucleotide sequence of SEQ ID NO: 905, or a nucleotide sequence at least 85%, 90%, 95%, or 99% identical or higher to SEQ ID NO: 905. In one embodiment, the antibody molecule comprises a VL encoded by the nucleotide sequence of SEQ ID NO: 906, or a nucleotide sequence at least 85%, 90%, 95%, or 99% identical or higher to SEQ ID NO: 906. In one embodiment, the antibody molecule comprises a VH encoded by the nucleotide sequence of SEQ ID NO: 905 and a VL encoded by the nucleotide sequence of SEQ ID NO: 906.

In one embodiment, the anti-GITR antibody molecule comprises a heavy chain comprising the amino acid sequence of SEQ ID NO: 903, or an amino acid sequence at least 85%, 90%, 95%, or 99% identical or higher to SEQ ID NO: 903. In one embodiment, the anti-GITR antibody molecule comprises a light chain comprising the amino acid sequence of SEQ ID NO: 904, or an amino acid sequence at least 85%, 90%, 95%, or 99% identical or higher to SEQ ID NO: 904. In one embodiment, the anti-GITR antibody molecule comprises a heavy chain comprising the amino acid sequence of SEQ ID NO: 903 and a light chain comprising the amino acid sequence of SEQ ID NO: 904.

In one embodiment, the antibody molecule comprises a heavy chain encoded by the nucleotide sequence of SEQ ID NO: 907, or a nucleotide sequence at least 85%, 90%, 95%, or 99% identical or higher to SEQ ID NO: 907. In one embodiment, the antibody molecule comprises a light chain encoded by the nucleotide sequence of SEQ ID NO: 908, or a nucleotide sequence at least 85%, 90%, 95%, or 99% identical or higher to SEQ ID NO: 908. In one embodiment, the antibody molecule comprises a heavy chain encoded by the nucleotide sequence of SEQ ID NO: 907 and a light chain encoded by the nucleotide sequence of SEQ ID NO: 908.

The antibody molecules described herein can be made by vectors, host cells, and methods described in WO 2016/057846, incorporated by reference in its entirety.

TABLE 13
Amino acid and nucleotide sequences of exemplary anti-GITR antibody molecule

| MAB7 | | |
|---|---|---|
| SEQ ID NO: 901 | VH | EVQLVESGGGLVQSGGSLRLSCAASGFSLSSYGVDWVRQAP |
| | | GKGLEWVGVIWGGGGTYYASSLMGRFTISRDNSKNTLYLQ |
| | | MNSLRAEDTAVYYCARHAYGHDGGFAMDYWGQGTLVTVS |
| | | S |
| SEQ ID NO: 902 | VL | EIVMTQSPATLSVSPGERATLSCRASESVSSNVAWYQQRPGQ |
| | | APRLLIYGASNRATGIPARFSGSGSGTDFTLTISRLEPEDFAVY |
| | | YCGQSYSYPFTFGQGTKLEIK |
| SEQ ID NO: 903 | Heavy | EVQLVESGGGLVQSGGSLRLSCAASGFSLSSYGVDWVRQAP |
| | Chain | GKGLEWVGVIWGGGGTYYASSLMGRFTISRDNSKNTLYLQ |
| | | MNSLRAEDTAVYYCARHAYGHDGGFAMDYWGQGTLVTVS |
| | | SASTKGPSVFPLAPSSKSTSGGTAALGCLVKDYFPEPVTVSW |
| | | NSGALTSGVHTFPAVLQSSGLYSLSSVVTVPSSSLGTQTYICN |
| | | VNHKPSNTKVDKRVEPKSCDKTHTCPPCPAPELLGGPSVFLF |
| | | PPKPKDTLMISRTPEVTCVVVDVSHEDPEVKFNWYVDGVEV |
| | | HNAKTKPREEQYNSTYRVVSVLTVLHQDWLNGKEYKCKVS |
| | | NKALPAPIEKTISKAKGQPREPQVYTLPPSREEMTKNQVSLTC |
| | | LVKGFYPSDIAVEWESNGQPENNYKTTPPVLDSDGSFFLYSK |
| | | LTVDKSRWQQGNVFSCSVMHEALHNHYTQKSLSLSPGK |
| SEQ ID NO: 904 | Light | EIVMTQSPATLSVSPGERATLSCRASESVSSNVAWYQQRPGQ |
| | Chain | APRLLIYGASNRATGIPARFSGSGSGTDFTLTISRLEPEDFAVY |
| | | YCGQSYSYPFTFGQGTKLEIKRTVAAPSVFIFPPSDEQLKSGT |
| | | ASVVCLLNNFYPREAKVQWKVDNALQSGNSQESVTEQDSK |
| | | DSTYSLSSTLTLSKADYEKHKVYACEVTHQGLSSPVTKSFNR |
| | | GEC |
| SEQ ID NO: 905 | DNA VH | GAGGTGCAGCTGGTGGAATCTGGCGGCGGACTGGTGCAG |
| | | TCCGGCGGCTCTCTGAGACTGTCTTGCGCTGCCTCCGGCTT |
| | | CTCCCTGTCCTCTTACGGCGTGGACTGGGTGCGACAGGCC |
| | | CCTGGCAAGGGCCTGGAATGGGTGGGAGTGATCTGGGGC |
| | | GGAGGCGGCACCTACTACGCCTCTTCCCTGATGGGCCGGT |
| | | TCACCATCTCCCGGGACAACTCCAAGAACACCCTGTACCT |
| | | GCAGATGAACTCCCTGCGGGCCGAGGACACCGCCGTGTAC |
| | | TACTGCGCCAGACACGCCTACGGCCACGACGGCGGCTTCG |
| | | CCATGGATTATTGGGGCCAGGGCACCCTGGTGACAGTGTC |
| | | CTCC |
| SEQ ID NO: 906 | DNA VL | GAGATCGTGATGACCCAGTCCCCCGCCACCCTGTCTGTGT |
| | | CTCCCGGCGAGAGAGCCACCCTGAGCTGCAGAGCCTCCGA |
| | | GTCCGTGTCCTCCAACGTGGCCTGGTATCAGCAGAGACCT |
| | | GGTCAGGCCCCTCGGCTGCTGATCTACGGCGCCTCTAACC |
| | | GGGCCACCGGCATCCCTGCCAGATTCTCCGGCTCCGGCAG |
| | | CGGCACCGACTTCACCCTGACCATCTCCCGGCTGGAACCC |
| | | GAGGACTTCGCCGTGTACTACTGCGGCCAGTCCTACTCAT |
| | | ACCCCTTCACCTTCGGCCAGGGCACCAAGCTGGAAATCAA |
| | | G |
| SEQ ID NO: 907 | DNA | GAGGTGCAGCTGGTGGAATCTGGCGGCGGACTGGTGCAG |
| | Heavy | TCCGGCGGCTCTCTGAGACTGTCTTGCGCTGCCTCCGGCTT |
| | Chain | CTCCCTGTCCTCTTACGGCGTGGACTGGGTGCGACAGGCC |
| | | CCTGGCAAGGGCCTGGAATGGGTGGGAGTGATCTGGGGC |
| | | GGAGGCGGCACCTACTACGCCTCTTCCCTGATGGGCCGGT |
| | | TCACCATCTCCCGGGACAACTCCAAGAACACCCTGTACCT |
| | | GCAGATGAACTCCCTGCGGGCCGAGGACACCGCCGTGTAC |
| | | TACTGCGCCAGACACGCCTACGGCCACGACGGCGGCTTCG |
| | | CCATGGATTATTGGGGCCAGGGCACCCTGGTGACAGTGTC |
| | | CTCCGCTAGCACCAAGGGCCCAAGTGTGTTTCCCCTGGCC |
| | | CCCAGCAGCAAGTCTACTTCCGGCGGAACTGCTGCCCTGG |
| | | GTTGCCTGGTGAAGGACTACTTCCCCGAGCCCGTGACAGT |
| | | GTCCTGGAACTCTGGGGCTCTGACTTCCGGCGTGCACACC |
| | | TTCCCCGCCGTGCTGCAGAGCAGCGGCCTGTACAGCCTGA |
| | | GCAGCGTGGTGACAGTGCCCTCCAGCTCTCTGGGAACCCA |
| | | GACCTATATCTGCAACGTGAACCACAAGCCCAGCAACACC |
| | | AAGGTGGACAAGAGAGTGGAGCCCAAGAGCTGCGACAAG |
| | | ACCCACACCTGCCCCCCCTGCCCAGCTCCAGAACTGCTGG |
| | | GAGGGCCTTCCGTGTTCCTGTTCCCCCCCAAGCCCAAGGA |
| | | CACCCTGATGATCAGCAGGACCCCCGAGGTGACCTGCGTG |
| | | GTGGTGGACGTGTCCCACGAGGACCCAGAGGTGAAGTTC |
| | | AACTGGTACGTGGACGGCGTGGAGGTGCACAACGCCAAG |
| | | ACCAAGCCCAGAGAGGAGCAGTACAACAGCACCTACAGG |
| | | GTGGTGTCCGTGCTGACCGTGCTGCACCAGGACTGGCTGA |
| | | ACGGCAAAGAATACAAGTGCAAAGTCTCCAACAAGGCCC |
| | | TGCCAGCCCCAATCGAAAAGACAATCAGCAAGGCCAAGG |
| | | GCCAGCCACGGGAGCCCCAGGTGTACACCCTGCCCCCCAG |
| | | CCGGGAGGAGATGACCAAGAACCAGGTGTCCCTGACCTG |
| | | TCTGGTGAAGGGCTTCTACCCCAGCGATATCGCCGTGGAG |
| | | TGGGAGAGCAACGGCCAGCCCGAGAACAACTACAAGACC |
| | | ACCCCCCCAGTGCTGGACAGCGACGGCAGCTTCTTCCTGT |
| | | ACAGCAAGCTGACCGTGGACAAGTCCAGGTGGCAGCAGG |
| | | GCAACGTGTTCAGCTGCAGCGTGATGCACGAGGCCCTGCA |
| | | CAACCACTACACCCAGAAGTCCCTGAGCCTGAGCCCCGGC |
| | | AAG |
| SEQ ID NO: 908 | DNA | GAGATCGTGATGACCCAGTCCCCCGCCACCCTGTCTGTGT |
| | Light | CTCCCGGCGAGAGAGCCACCCTGAGCTGCAGAGCCTCCGA |
| | Chain | GTCCGTGTCCTCCAACGTGGCCTGGTATCAGCAGAGACCT |
| | | GGTCAGGCCCCTCGGCTGCTGATCTACGGCGCCTCTAACC |
| | | GGGCCACCGGCATCCCTGCCAGATTCTCCGGCTCCGGCAG |
| | | CGGCACCGACTTCACCCTGACCATCTCCCGGCTGGAACCC |
| | | GAGGACTTCGCCGTGTACTACTGCGGCCAGTCCTACTCAT |
| | | ACCCCTTCACCTTCGGCCAGGGCACCAAGCTGGAAATCAA |
| | | GCGTACGGTGGCCGCTCCCAGCGTGTTCATCTTCCCCCCC |
| | | AGCGACGAGCAGCTGAAGAGCGGCACCGCCAGCGTGGTG |
| | | TGCCTGCTGAACAACTTCTACCCCCGGGAGGCCAAGGTGC |
| | | AGTGGAAGGTGGACAACGCCCTGCAGAGCGGCAACAGCC |
| | | AGGAGAGCGTCACCGAGCAGGACAGCAAGGACTCCACCT |
| | | ACAGCCTGAGCAGCACCCTGACCCTGAGCAAGGCCGACT |
| | | ACGAGAAGCATAAGGTGTACGCCTGCGAGGTGACCCACC |
| | | AGGGCCTGTCCAGCCCCGTGACCAAGAGCTTCAACAGGG |
| | | GCGAGTGC |
| SEQ ID NO: 909 (KABAT) | HCDR1 | SYGVD |
| SEQ ID NO: 910 (CHOTHIA) | HCDR1 | GFSLSSY |
| SEQ ID NO: 911 (KABAT) | HCDR2 | VIWGGGGTYYASSLMG |
| SEQ ID NO: 912 (CHOTHIA) | HCDR2 | WGGGG |
| SEQ ID NO: 913 (KABAT) | HCDR3 | HAYGHDGGFAMDY |
| SEQ ID NO: 913 (CHOTHIA) | HCDR3 | HAYGHDGGFAMDY |
| SEQ ID NO: 914 (KABAT) | LCDR1 | RASESVSSNVA |
| SEQ ID NO: 915 (CHOTHIA) | LCDR1 | SESVSSN |
| SEQ ID NO: 916 (KABAT) | LCDR2 | GASNRAT |
| SEQ ID NO: 917 (CHOTHIA) | LCDR2 | GAS |
| SEQ ID NO: 918 (KABAT) | LCDR3 | GQSYSYPFT |
| SEQ ID NO: 919 (CHOTHIA) | LCDR3 | SYSYPF |

### Other Exemplary GITR Agonists

In one embodiment, the anti-GITR antibody molecule is BMS-986156 (Bristol-Myers Squibb), also known as BMS 986156 or BMS986156. BMS-986156 and other anti-GITR antibodies are disclosed, e.g., in U.S. Pat. No. 9,228,016 and WO 2016/196792, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of BMS-986156, e.g., as disclosed in Table 14.

In one embodiment, the anti-GITR antibody molecule is MK-4166 or MK-1248 (Merck). MK-4166, MK-1248, and other anti-GITR antibodies are disclosed, e.g., in U.S. Pat. No. 8,709,424, WO 2011/028683, WO 2015/026684, and Mahne et al. Cancer Res. 2017; 77(5):1108-1118, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of MK-4166 or MK-1248.

In one embodiment, the anti-GITR antibody molecule is TRX518 (Leap Therapeutics). TRX518 and other anti-GITR antibodies are disclosed, e.g., in U.S. Pat. Nos. 7,812,135, 8,388,967, 9,028,823, WO 2006/105021, and Ponte J et al. (2010) Clinical Immunology; 135:S96, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of TRX518.

In one embodiment, the anti-GITR antibody molecule is INCAGN1876 (Incyte/Agenus). INCAGN1876 and other anti-GITR antibodies are disclosed, e.g., in US 2015/0368349 and WO 2015/184099, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of INCAGN1876.

In one embodiment, the anti-GITR antibody molecule is AMG 228 (Amgen). AMG 228 and other anti-GITR antibodies are disclosed, e.g., in U.S. Pat. No. 9,464,139 and WO 2015/031667, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of AMG 228.

In one embodiment, the anti-GITR antibody molecule is INBRX-110 (Inhibrx). INBRX-110 and other anti-GITR antibodies are disclosed, e.g., in US 2017/0022284 and WO 2017/015623, incorporated by reference in their entirety. In one embodiment, the GITR agonist comprises one or more of the CDR sequences (or collectively all of the CDR sequences), the heavy chain or light chain variable region sequence, or the heavy chain or light chain sequence of INBRX-110.

In one embodiment, the GITR agonist (e.g., a fusion protein) is MEDI 1873 (MedImmune), also known as MEDI1873. MEDI 1873 and other GITR agonists are disclosed, e.g., in US 2017/0073386, WO 2017/025610, and Ross et al. Cancer Res 2016; 76 (14 Suppl): Abstract nr 561, incorporated by reference in their entirety. In one embodiment, the GITR agonist comprises one or more of an IgG Fc domain, a functional multimerization domain, and a receptor binding domain of a glucocorticoid-induced TNF receptor ligand (GITRL) of MEDI 1873.

Further known GITR agonists (e.g., anti-GITR antibodies) include those described, e.g., in WO 2016/054638, incorporated by reference in its entirety.

In one embodiment, the anti-GITR antibody is an antibody that competes for binding with, and/or binds to the same epitope on GITR as, one of the anti-GITR antibodies described herein.

In one embodiment, the GITR agonist is a peptide that activates the GITR signaling pathway. In one embodiment, the GITR agonist is an immunoadhesin binding fragment (e.g., an immunoadhesin binding fragment comprising an extracellular or GITR binding portion of GITRL) fused to a constant region (e.g., an Fc region of an immunoglobulin sequence).

TABLE 14
Amino acid sequences of other exemplary
anti-GITR antibody molecules

| BMS-986156 | | |
|---|---|---|
| SEQ ID NO: 920 | VH | QVQLVESGGGVVQPGRSLRLSCAASGFTFSSYGMHWVRQAPGKGL |
| | | EWVAVIWYEGSNKYYADSVKGRFTISRDNSKNTLYLQMNSLRAED |
| | | TAVYYCARGGSMVRGDYYYGMDVWGQGTTVTVSS |
| SEQ ID NO: 921 | VL | AIQLTQSPSSLSASVGDRVTITCRASQGISSALAWYQQKPGKAPKLLI |
| | | YDASSLESGVPSRFSGSGSGTDFTLTISSLQPEDFATYYCQQFNSYPY |
| | | TFGQGTKLEIK |

### Exemplary Anti-CD3 Multispecific Antibody Molecules

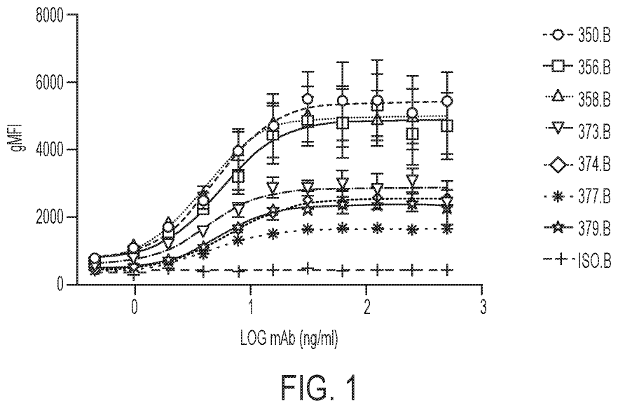

In certain embodiments, the anti-CD73 antibody molecule described herein is administered in combination with an anti-CD3 multispecific antibody molecule (e.g., an anti-CD3 bispecific antibody molecule). In one embodiment, the anti-CD3 multispecific antibody molecule binds to CD3 and a target tumor antigen (TTA). In one embodiment, the TTA is chosen from CD19, CD20, CD38, or CD123. In one embodiment, the anti-CD3 multispecific antibody molecule is in a format disclosed in FIGS. 1A, 1B, 1C, and 125 of WO 2016/182751, herein incorporated by reference in its entirety.

In one embodiment, the anti-CD3 multispecific antibody molecule is an anti-CD3×anti-CD123 bispecific antibody molecule, e.g., XENP14045 (e.g., as set out in Table 15) or an anti-CD3×anti-CD123 bispecific antibody molecule disclosed in WO 2016/086189 or WO 2016/182751, herein incorporated by reference in their entirety. In one embodiment, the anti-CD3×anti-CD123 bispecific antibody molecule comprises one or more of the CDR sequences (or collectively all of the CDR sequences), the heavy chain or light chain variable region sequence, or the heavy chain or light chain sequence of XENP14045, or an amino acid sequence substantially identical thereto (e.g., a sequence having at least about 85%, 90%, or 95% sequence identity thereto).

In one embodiment, the anti-CD3 multispecific antibody is an anti-CD3×anti-CD20 bispecific antibody molecule, e.g., XENP13676 (e.g., as set out in Table 15) or an anti-CD3×anti-CD20 bispecific antibody molecule disclosed in WO 2016/086189 or WO 2016/182751, herein incorporated by reference in their entirety. In one embodiment, the anti-CD3×anti-CD20 bispecific antibody molecule comprises one or more of the CDR sequences (or collectively all of the CDR sequences), the heavy chain or light chain variable region sequence, or the heavy chain or light chain sequence of XENP13676, or an amino acid sequence substantially identical thereto (e.g., a sequence having at least about 85%, 90%, or 95% sequence identity thereto).

TABLE 15
Amino acid sequences of exemplary anti-CD3 bispecific antibody molecules

| | | |
|---|---|---|
| XENP14045 (anti- | | |
| CD123 x anti-CD3 | | |
| Fab-scFv-Fc) | | |
| SEQ ID NO: 177 | Heavy chain | QVQLQQSGAEVKKPGASVKVSCKASGYTFTDYYMKWVKQSHGKS |
| | 1 (anti- | LEWMGDIIPSNGATFYNQKFKGKATLTVDRSTSTAYMELSSLRSED |
| | CD123 Fab- | TAVYYCARSHLLRASWFAYWGQGTLVTVSSASTKGPSVFPLAPSSK |
| | Fc) | STSGGTAALGCLVKDYFPEPVTVSWNSGALTSGVHTFPAVLQSSGL |
| | | YSLSSVVTVPSSSLGTQTYICNVNHKPSDTKVDKKVEPKSCDKTHT |
| | | CPPCPAPPVAGPSVFLFPPKPKDTLMISRTPEVTCVVVDVKHEDPEV |
| | | KFNWYVDGVEVHNAKTKPREEEYNSTYRVVSVLTVLHQDWLNGK |
| | | EYKCKVSNKALPAPIEKTISKAKGQPREPQVYTLPPSREEMTKNQVS |
| | | LTCDVSGFYPSDIAVEWESDGQPENNYKTTPPVLDSDGSFFLYSKLT |
| | | VDKSRWEQGDVFSCSVMHEALHNHYTQKSLSLSPGK |
| SEQ ID NO: 178 | Heavy chain | EVQLVESGGGLVQPGGSLRLSCAASGFTFSTYAMNWVRQAPGKGL |
| | 2 (anti-CD3 | EWVGRIRSKYNNYATYYADSVKGRFTISRDDSKNTLYLQMNSLRA |
| | scFv-Fc) | EDTAVYYCVRHGNFGDSYVSWFAYWGQGTLVTVSSGKPGSGKPG |
| | | SGKPGSGKPGSQAVVTQEPSLTVSPGGTVTLTCGSSTGAVTTSNYA |
| | | NWVQQKPGKSPRGLIGGTNKRAPGVPARFSGSLLGGKAALTISGAQ |
| | | PEDEADYYCALWYSNHWVFGGGTKLTVLEPKSSDKTHTCPPCPAP |
| | | PVAGPSVFLFPPKPKDTLMISRTPEVTCVVVDVKHEDPEVKFNWYV |
| | | DGVEVHNAKTKPREEQYNSTYRVVSVLTVLHQDWLNGKEYKCKV |
| | | SNKALPAPIEKTISKAKGQPREPQVYTLPPSREQMTKNQVKLTCLVK |
| | | GFYPSDIAVEWESNGQPENNYKTTPPVLDSDGSFFLYSKLTVDKSR |
| | | WQQGNVFSCSVMHEALHNHYTQKSLSLSPGK |
| SEQ ID NO: 179 | Light chain | DFVMTQSPDSLAVSLGERATINCKSSQSLLNTGNQKNYLTWYQQKP |
| | | GQPPKLLIYWASTRESGVPDRFTGSGSGTDFTLTISSLQAEDVAVYY |
| | | CQNDYSYPYTFGGGTKLEIKRTVAAPSVFIFPPSDEQLKSGTASVVC |
| | | LLNNFYPREAKVQWKVDNALQSGNSQESVTEQDSKDSTYSLSSTLT |
| | | LSKADYEKHKVYACEVTHQGLSSPVTKSFNRGEC |
| XENP13676 (anti- | | |
| CD20 x anti-CD3 | | |
| Fab-scFv-Fc) | | |
| SEQ ID NO: 180 | Heavy chain | QVQLVQSGAEVKKPGASVKVSCKASGYTFTSYNMHWVRQAPGQG |
| | 1 (Fab-Fc) | LEWMGAIYPGNGDTSYNQKFQGRVTITADKSISTAYMELSSLRSED |
| | | TAVYYCARSTYYGGDWYFNVWGAGTLVTVSSASTKGPSVFPLAPS |
| | | SKSTSGGTAALGCLVKDYFPEPVTVSWNSGALTSGVHTFPAVLQSS |
| | | GLYSLSSVVTVPSSSLGTQTYICNVNHKPSDTKVDKKVEPKSCDKT |
| | | HTCPPCPAPPVAGPSVFLFPPKPKDTLMISRTPEVTCVVVDVKHEDP |
| | | EVKFNWYVDGVEVHNAKTKPREEEYNSTYRVVSVLTVLHQDWLN |
| | | GKEYKCKVSNKALPAPIEKTISKAKGQPREPQVYTLPPSREEMTKN |
| | | QVSLTCDVSGFYPSDIAVEWESDGQPENNYKTTPPVLDSDGSFFLYS |
| | | KLTVDKSRWEQGDVFSCSVMHEALHNHYTQKSLSLSPGK |
| SEQ ID NO: 181 | Heavy chain | EVQLVESGGGLVQPGGSLRLSCAASGFTFSTYAMNWVRQAPGKGL |
| | 2 (scFv-Fc) | EWVGRIRSKYNNYATYYADSVKGRFTISRDDSKNTLYLQMNSLRA |
| | | EDTAVYYCVRHGNFGDSYVSWFAYWGQGTLVTVSSGKPGSGKPG |
| | | SGKPGSGKPGSQAVVTQEPSLTVSPGGTVTLTCGSSTGAVTTSNYA |
| | | NWVQQKPGKSPRGLIGGTNKRAPGVPARFSGSLLGGKAALTISGAQ |
| | | PEDEADYYCALWYSNHWVFGGGTKLTVLEPKSSDKTHTCPPCPAP |
| | | PVAGPSVFLFPPKPKDTLMISRTPEVTCVVVDVKHEDPEVKFNWYV |
| | | DGVEVHNAKTKPREEQYNSTYRVVSVLTVLHQDWLNGKEYKCKV |
| | | SNKALPAPIEKTISKAKGQPREPQVYTLPPSREQMTKNQVKLTCLVK |
| | | GFYPSDIAVEWESNGQPENNYKTTPPVLDSDGSFFLYSKLTVDKSR |
| | | WQQGNVFSCSVMHEALHNHYTQKSLSLSPGK |
| SEQ ID NO: 182 | Light chain | QIVLTQSPSSLSASVGDRVTITCRASSSVSYIHWFQQKPGKSPKPLIY |
| | | ATSNLASGVPVRFSGSGSGTDYTLTISSLQPEDFATYYCQQWTSNPP |
| | | TFGGGTKVEIKRTVAAPSVFIFPPSDEQLKSGTASVVCLLNNFYPRE |
| | | AKVQWKVDNALQSGNSQESVTEQDSKDSTYSLSSTLTLSKADYEK |
| | | HKVYACEVTHQGLSSPVTKSFNRGEC |

### Exemplary IL15/IL-15Ra Complexes

In certain embodiments, the anti-CD73 antibody molecule described herein is administered in combination with an IL-15/IL-15Ra complex. In some embodiments, the IL-15/IL-15Ra complex is chosen from NIZ985 (Novartis), ATL-803 (Altor) or CYP0150 (Cytune).

### Exemplary IL-15/IL-15Ra Complexes

In one embodiment, the IL-15/IL-15Ra complex comprises human IL-15 complexed with a soluble form of human IL-15Ra. The complex may comprise IL-15 covalently or noncovalently bound to a soluble form of IL-15Ra. In a particular embodiment, the human IL-15 is noncovalently bonded to a soluble form of IL-15Ra. In a particular embodiment, the human IL-15 of the composition comprises an amino acid sequence of SEQ ID NO: 183 in Table 16 and the soluble form of human IL-15Ra comprises an amino acid sequence of SEQ ID NO: 184 in Table 16, as described in WO 2014/066527, incorporated by reference in its entirety. The molecules described herein can be made by vectors, host cells, and methods described in WO 2007/084342, incorporated by reference in its entirety.

TABLE 16
Amino acid and nucleotide
sequences of exemplary IL-15/IL-15Ra complexes
NIZ985

| | | |
|---|---|---|
| SEQ ID NO: | Human IL-15 | NWVNVISDLKKIEDLIQSMHIDATLYTESDVHPSCKVTAMKCFLLEL |
| 183 | | QVISLESGDASIHDTVENLIILANNSLSSNGNVTESGCKECEELEEKNI |
| | | KEFLQSFVHIVQMFINTS |
| SEQ ID NO: | Human Soluble | ITCPPPMSVEHADIWVKSYSLYSRERYICNSGFKRKAGTSSLTECVLN |
| 184 | IL-15Ra | KATNVAHWTTPSLKCIRDPALVHQRPAPPSTVTTAGVTPQPESLSPSG |
| | | KEPAASSPSSNNTAATTAAIVPGSQLMPSKSPSTGTTEISSHESSHGTPS |
| | | QTTAKNWELTASASHQPPGVYPQG |

### Other Exemplary IL-15/IL-15Ra Complexes

In one embodiment, the IL-15/IL-15Ra complex is ALT-803, an IL-15/IL-15Ra Fc fusion protein (IL-15N72D:IL-15RaSu/Fc soluble complex). ALT-803 is disclosed in WO 2008/143794, incorporated by reference in its entirety. In one embodiment, the IL-15/IL-15Ra Fc fusion protein comprises the sequences as disclosed in Table 17.

In one embodiment, the IL-15/IL-15Ra complex comprises IL-15 fused to the sushi domain of IL-15Ra (CYP0150, Cytune). The sushi domain of IL-15Ra refers to a domain beginning at the first cysteine residue after the signal peptide of IL-15Ra, and ending at the fourth cysteine residue after said signal peptide. The complex of IL-15 fused to the sushi domain of IL-15Ra is disclosed in WO 2007/04606 and WO 2012/175222, incorporated by reference in their entirety. In one embodiment, the IL-15/IL-15Ra sushi domain fusion comprises the sequences as disclosed in Table 17.

TABLE 17
Amino acid sequences of other exemplary IL-15/IL-15Ra complexes
ALT-803 (Altor)

| | | |
|---|---|---|
| SEQ ID NO: | IL-15N72D | NWVNVISDLKKIEDLIQSMHIDATLYTESDVHPSCKVTAMKCFLLEL |
| 185 | | QVISLESGDASIHDTVENLIILANDSLSSNGNVTESGCKECEELEEKNI |
| | | KEFLQSFVHIVQMFINTS |
| SEQ ID NO: | IL-15RaSu/Fc | ITCPPPMSVEHADIWVKSYSLYSRERYICNSGFKRKAGTSSLTECVLN |
| 186 | | KATNVAHWTTPSLKCIREPKSCDKTHTCPPCPAPELLGGPSVFLFPPK |
| | | PKDTLMISRTPEVTCVVVDVSHEDPEVKFNWYVDGVEVHNAKTKPR |
| | | EEQYNSTYRVVSVLTVLHQDWLNGKEYKCKVSNKALPAPIEKTISKA |
| | | KGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEAL |
| | | HNHYTQKSLSLSPGK |

IL-15/IL-15Ra sushi domain fusion (Cytune)

| | | |
|---|---|---|
| SEQ ID NO: | Human IL-is | NWVNVISDLKKIEDLIQSMHIDATLYTESDVHPSCKVTAMKCPLLEL |
| 187 | | QVISLESGDASIHDTVENLIILANNSLSSNGNVTESGCKECEELEXKNI |
| | | KEFLQSFVHIVQMFINTS |
| | | Where X is E or K |
| SEQ ID NO: | Human IL- | ITCPPPMSVEHADIWVKSYSLYSRERYICNSGFKRKAGTSSLTECVLN |
| 188 | 15Ra sushi and | KATNVAHWTTPSLKCIRDPALVHQRPAPP |
| | hinge domains | |

### Exemplary STING Agonists

In certain embodiments, the anti-CD73 antibody molecule described herein is administered in combination with a STING agonist. In some embodiments, the combination is used to treat a cancer, e.g., a cancer described herein e.g., a solid tumor (e.g., a breast cancer, a squamous cell carcinoma, a melanoma, an ovarian cancer, a fallopian tube carcinoma, a peritoneal carcinoma, a soft tissue sarcoma, an esophageal cancer, a head and neck cancer, an endometrial cancer, a cervical cancer, or a basal cell carcinoma), e.g., a hematologic malignancy (e.g., a leukemia (e.g., a chronic lymphocytic leukemia (CLL), or a lymphoma (e.g., a marginal zone B-cell lymphoma, a small lymphocytic lymphoma, a follicular lymphoma, Hodgkin lymphoma, non-Hodgkin lymphoma)). In some embodiments, the cancer is chosen from a head and neck cancer (e.g., a head and neck squamous cell carcinoma (HNSCC), a skin cancer (e.g., melanoma), a lung cancer (e.g., a non-small cell lung cancer (NSCLC)), or a breast cancer (e.g., a triple negative breast cancer (TNBC)).

In some embodiments, the STING agonist is cyclic dinucleotide, e.g., a cyclic dinucleotide comprising purine or pyrimidine nucleobases (e.g., adenosine, guanine, uracil, thymine, or cytosine nucleobases). In some embodiments, the nucleobases of the cyclic dinucleotide comprise the same nucleobase or different nucleobases.

In some embodiments, the STING agonist comprises an adenosine or a guanosine nucleobase. In some embodiments, the STING agonist comprises one adenosine nucleobase and one guanosine nucleobase. In some embodiments, the STING agonist comprises two adenosine nucleobases or two guanosine nucleobases.

In some embodiments, the STING agonist comprises a modified cyclic dinucleotide, e.g., comprising a modified nucleobase, a modified ribose, or a modified phosphate linkage. In some embodiments, the modified cyclic dinucleotide comprises a modified phosphate linkage, e.g., a thiophosphate.

In some embodiments, the STING agonist comprises a cyclic dinucleotide (e.g., a modified cyclic dinucleotide) with 2′,5′ or 3′,5′ phosphate linkages. In some embodiments, the STING agonist comprises a cyclic dinucleotide (e.g., a modified cyclic dinucleotide) with Rp or Sp stereochemistry around the phosphate linkages.

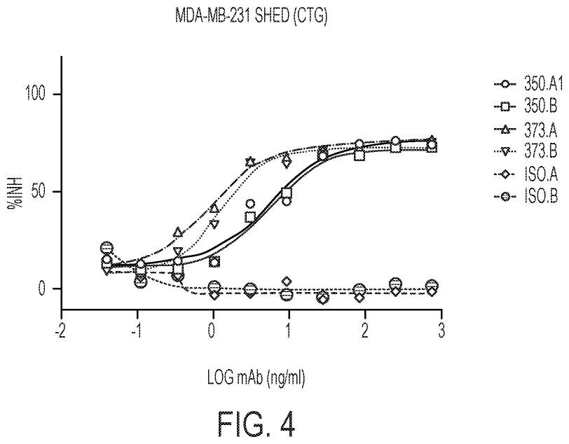

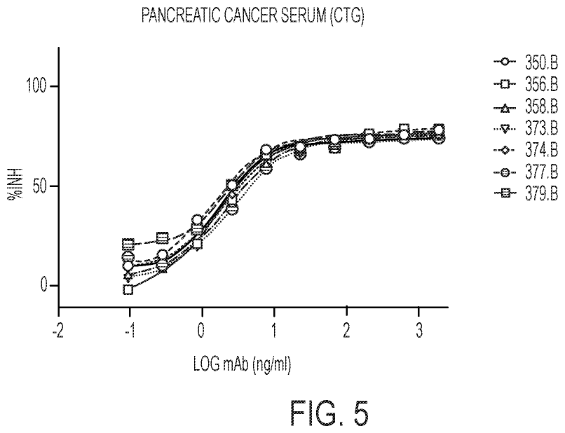

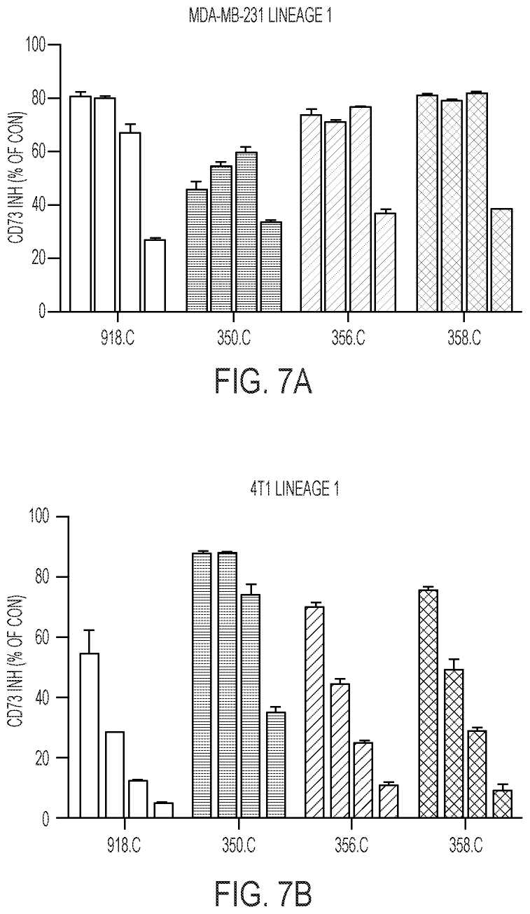

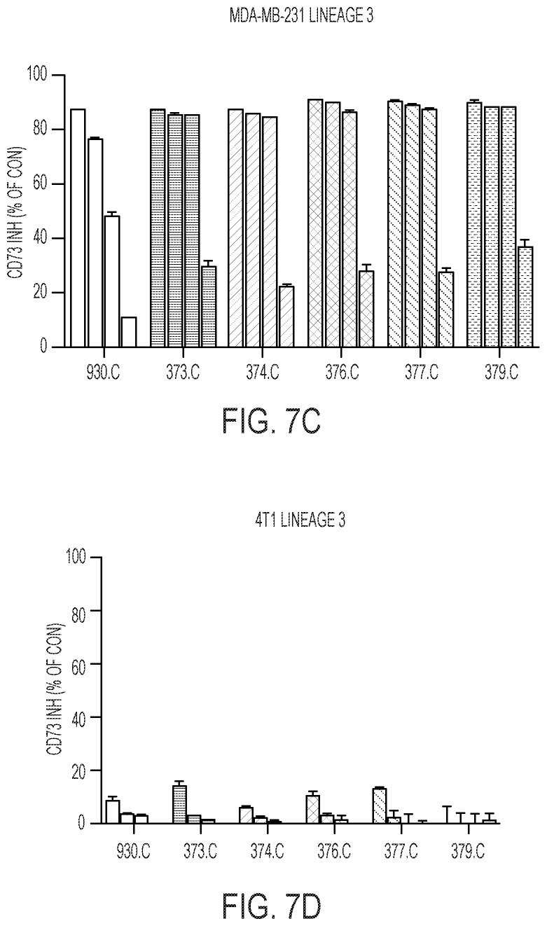

In some embodiments, the STING agonist is Rp,Rp dithio 2′,3′ c-di-AMP (e.g., Rp,Rp-dithio c-[A(2′,5′)pA(3′,5′)p]), or a cyclic dinucleotide analog thereof. In some embodiments, the STING agonist is a compound depicted in U.S. Patent Publication No. US2015/0056224 (e.g., a compound in FIG. 2c, e.g., compound 21 or compound 22). In some embodiments, the STING agonist is c-[G(2′,5′)pG(3′,5′)p], a dithio ribose O-substituted derivative thereof, or a compound depicted in FIG. 4 of PCT Publication Nos. WO 2014/189805 and WO 2014/189806. In some embodiments, the STING agonist is c-[A(2′,5′)pA(3′,5′)p] or a dithio ribose O-substituted derivative thereof, or is a compound depicted in FIG. 5 of PCT Publication Nos. WO 2014/189805 and WO 2014/189806. In some embodiments, the STING agonist is c4[G(2′,5′)pA(3′,5′)p], or a dithio ribose O-substituted derivative thereof, or is a compound depicted in FIG. 5 of PCT Publication Nos. WO 2014/189805 and WO 2014/189806. In some embodiments, the STING agonist is 2′-O-propargyl-cyclic[A(2′,5′)pA(3′,5′)p] (2′-O-propargyl-ML-CDA) or a compound depicted in FIG. 7 of PCT Publication No. WO 2014/189806.

Other exemplary STING agonists are disclosed, e.g., in PCT Publication Nos. WO 2014/189805 and WO 2014/189806, and U.S. Publication No. 2015/0056225.

### Exemplary CSF-1/1R Binding Agents

In certain embodiments, the anti-CD73 antibody molecule described herein is administered in combination with a CSF-1/1R binding agent. In some embodiments, the combination is used to treat a cancer, e.g., a cancer described herein, e.g., a solid tumor (e.g., a prostate cancer, a breast cancer, or pigmented villonodular synovitis (PVNS)).

In some embodiments, the CSF-1/1R binding agent is an inhibitor of macrophage colony-stimulating factor (M-CSF). M-CSF is also sometimes known as CSF-1.

In another embodiment, the CSF-1/1R binding agent is a CSF-1R tyrosine kinase inhibitor, 4-((2-(((1R,2R)-2-hydroxycyclohexyl)amino)benzo[d]thiazol-6-yl)oxy)-N-methylpicolinamide (Compound A15), or a compound disclosed in PCT Publication No. WO 2005/073224. In some embodiments, the cancer is chosen from a brain cancer (e.g., glioblastoma multiforme (GBM)), a pancreatic cancer, or a breast cancer (e.g., a triple-negative breast cancer (TNBC)).

In some embodiments, the CSF-1/1R binding agent (e.g., a CSF-1R tyrosine kinase inhibitor), 4-((2-(((1R,2R)-2-hydroxycyclohexyl)amino)benzo[d]thiazol-6-yl)oxy)-N-methylpicolinamide (Compound A15), or a compound disclosed in PCT Publication No. WO 2005/073224, is administered in combination with a CD73 inhibitor (e.g., an anti-CD73 antibody molecule).

In certain embodiments, the CSF-1/1R binding agent (e.g., a CSF-1R tyrosine kinase inhibitor), 4-((2-(((1R,2R)-2-hydroxycyclohexyl)amino)benzo[d]thiazol-6-yl)oxy)-N-methylpicolinamide (Compound A15), or a compound disclosed in PCT Publication No. WO 2005/073224, is administered in combination with the CD73 inhibitor (e.g., the anti-CD73 antibody molecule) to treat a caner, e.g., a solid tumor (e.g., an advanced solid tumor), e.g., a brain cancer (e.g., glioblastoma multiforme (GBM), e.g., recurrent glioblastoma), a breast cancer (e.g., a triple-negative breast cancer (e.g., NTBC)), or a pancreatic cancer (e.g., advanced pancreatic cancer).

In some embodiments, the CSF-1/1R binding agent is an M-CSF inhibitor, Compound A33, or a binding agent to CSF-1 disclosed in PCT Publication No. WO 2004/045532 or PCT Publication No WO 2005/068503 including RX1 or 5H4 (e.g., an antibody molecule or Fab fragment against M-CSF). In some embodiments, the cancer is chosen from an endometrial cancer, a skin cancer (e.g., melanoma), a pancreatic cancer, or a breast cancer (e.g., a triple negative breast cancer (TNBC)).

In some embodiments, the CSF-1/1R binding agent is a CSF1R inhibitor or 4-(2-((1R, 2R)-2-hydroxycyclohexylamino)benzothiazol-6-yloxy)-N-methylpicolinamide. 4-(2-((1R, 2R)-2-hydroxycyclohexylamino)benzothiazol-6-yloxy)-N-methylpicolinamide is disclosed as example 157 at page 117 of PCT Publication No. WO 2007/121484.

In some embodiments, the CSF-1/1R binding agent is pexidartinib (CAS Registry Number 1029044-16-3). Pexidrtinib is also known as PLX3397 or 5-((5-chloro-1H-pyrrolo[2,3-b]pyridin-3-yl)methyl)-N-((6-(trifluoromethyl)pyridin-3-yl)methyl)pyridin-2-amine Pexidartinib is a small-molecule receptor tyrosine kinase (RTK) inhibitor of KIT, CSF1R and FLT3. In some embodiments, the CSF-1/1R binding agent, e.g., pexidartinib, is used in combination with a CD73 inhibitor, e.g., an anti-CD73 antibody molecule described herein.

In some embodiments, the CSF-1/1R binding agent is emactuzumab. Emactuzumab is also known as RG7155 or R05509554. Emactuzumab is a humanized IgG1 mAb targeting CSF1R. In some embodiments, the CSF-1/1R binding agent, e.g., pexidartinib, is used in combination with a CD73 inhibitor, e.g., an anti-CD73 antibody molecule described herein.

In some embodiments, the CSF-1/1R binding agent is FPA008. FPA008 is a humanized mAb that inhibits CSF1R. In some embodiments, the CSF-1/1R binding agent, e.g., FPA008, is used in combination with a CD73 inhibitor, e.g., an anti-CD73 antibody molecule described herein.

### Exemplary IDO/TDO Inhibitors

In certain embodiments, the anti-CD73 antibody molecule described herein is administered in combination with an inhibitor of indoleamine 2,3-dioxygenase (IDO) and/or tryptophan 2,3-dioxygenase (TDO). In some embodiments, the combination is used to treat a cancer, e.g., a cancer described herein, e.g., a solid tumor (e.g., melanoma, non-small cell lung cancer, colon cancer, squamous cell head and neck cancer, ovarian cancer, peritoneal cancer, fallopian tube cancer, breast cancer (e.g., metastatic or HER2-negative breast cancer)), e.g., a hematologic malignancy (e.g., a lymphoma, e.g., a non-Hodgkin's lymphoma or a Hodgkin's lymphoma (e.g., a diffuse large B-cell lymphoma (DLBCL))).

In some embodiments, the IDO/TDO inhibitor is chosen from (4E)-4-[(3-chloro-4-fluoroanilino)-nitrosomethylidene]-1,2,5-oxadiazol-3-amine (also known as INCB24360), indoximod (1-methyl-D-tryptophan), or a-cyclohexyl-5H-Imidazo[5,1-a]isoindole-5-ethanol (also known as NLG919).

In some embodiments, the IDO/TDO inhibitor is epacadostat (CAS Registry Number: 1204669-58-8). Epacadostat is also known as INCB24360 or INCB024360 (Incyte). Epacadostat is a potent and selective indoleamine 2,3-dioxygenase (IDO1) inhibitor with IC50 of 10 nM, highly selective over other related enzymes such as IDO2 or tryptophan 2,3-dioxygenase (TDO).

In some embodiments, the IDO/TDO inhibitor is indoximod (New Link Genetics). Indoximod, the D isomer of 1-methyl-tryptophan, is an orally administered small-molecule indoleamine 2,3-dioxygenase (IDO) pathway inhibitor that disrupts the mechanisms by which tumors evade immune-mediated destruction.

In some embodiments, the IDO/TDO inhibitor is NLG919 (New Link Genetics). NLG919 is a potent IDO (indoleamine-(2,3)-dioxygenase) pathway inhibitor with Ki/EC50 of 7 nM/75 nM in cell-free assays.

In some embodiments, the IDO/TDO inhibitor is F001287 (Flexus/BMS). F001287 is a small molecule inhibitor of indoleamine 2,3-dioxygenase 1 (IDO1).

### Exemplary TGF-β Inhibitors

In certain embodiments, the anti-CD73 antibody molecule described herein is administered in combination with a transforming growth factor beta (TGF-β) inhibitor. In some embodiments, the combination is used to treat a cancer, e.g., a cancer described herein, e.g., a solid tumor (e.g., a brain cancer (e.g., a glioma), a melanoma, a kidney cancer (e.g., a renal cell carcinoma), a pleural malignant mesothelioma (e.g., a relapsed pleural malignant mesothelioma), or a breast cancer (e.g., a metastatic breast cancer)). In certain embodiments, the cancer is chosen from a colorectal cancer (e.g., a microsatelliate stable colorectal cancer (MSS CRC), a liver cancer (e.g., a hepatocellular carcinoma), a lung cancer (e.g., a non-small cell lung cancer (HSCLC)), a breast cancer (e.g., a triple negative breast cancer (TNBC)), a TGF-β-expressing cancer, a pancreatic cancer, a prostate cancer, or a renal cancer (e.g., a renal cell carcinoma).

TGF-β belongs to a large family of structurally-related cytokines including, e.g., bone morphogenetic proteins (BMPs), growth and differentiation factors, activins and inhibins. In some embodiments, the TGF-β inhibitors described herein can bind and/or inhibit one or more isoforms of TGF-β (e.g., one, two, or all of TGF-β1, TGF-β2, or TGF-β3).

In some embodiments, the TGF-β inhibitor is fresolimumab (CAS Registry Number: 948564-73-6). Fresolimumab is also known as GC1008. Fresolimumab is a human monoclonal antibody that binds to and inhibits TGF-beta isoforms 1, 2 and 3.

The heavy chain of fresolimumab has the amino acid sequence of:

(SEQ ID NO: 172)
QVQLVQSGAEVKKPGSSVKVSCKASGYTFSSNVISWVRQAPGQGLEWMGG
VIPIVDIANYAQRFKGRVTITADESTSTTYMELSSLRSEDTAVYYCASTL
GLVLDAMDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPSCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK.

The light chain of fresolimumab has the amino acid sequence of:

(SEQ ID NO: 173)
ETVLTQSPGTLSLSPGERATLSCRASQSLGSSYLAWYQQKPGQAPRLLIY
GASSRAPGIPDRFSGSGSGTDFTLTISRLEPEDFAVYYCQQYADSPITFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

Fresolimumab is disclosed, e.g., in WO 2006/086469, U.S. Pat. Nos. 8,383,780, and 8,591,901.

In some embodiments, the TGF-β inhibitor is XOMA 089. XOMA 089 is also known as XPA.42.089. XOMA 089 is a fully human monoclonal antibody that binds and neutralizes TGF-beta 1 and 2 ligands.

The heavy chain variable region of XOMA 089 has the amino acid sequence of:

(SEQ ID NO: 174)
QVQLVQSGAEVKKPGSSVKVSCKASGGTFSSYAISWVRQAPGQGLEWMGG
IIPIFGTANYAQKFQGRVTITADESTSTAYMELSSLRSEDTAVYYCARGL
WEVRALPSVYWGQGTLVTVSS
(disclosed as SEQ ID NO: 6 in WO 2012/167143).

The light chain variable region of XOMA 089 has the amino acid sequence of:

(SEQ ID NO: 175)
SYELTQPPSVSVAPGQTARITCGANDIGSKSVHWYQQKAGQAPVLVVSED
IIRPSGIPERISGSNSGNTATLTISRVEAGDEADYYCQVWDRDSDQYVFG
TGTKVTVLG
(disclosed as SEQ ID NO: 8 in WO 2012/167143).

In certain embodiments, the combination includes an inhibitor of CD73 (e.g., an anti-CD73 antibody molecule described herein) and a TGF-β inhibitor (e.g., a TGF-β inhibitor described herein).

In one embodiment, the combination includes a TGF-β inhibitor, XOMA 089, or a compound disclosed in PCT Publication No. WO 2012/167143, and an inhibitor of CD73 (e.g., an anti-CD73 antibody described herein).

In one embodiment, the TGF-β inhibitor, XOMA 089, or a compound disclosed in PCT Publication No. WO 2012/167143, is administered in combination with an inhibitor of CD73 (e.g., an anti-CD73 antibody molecule) to treat a pancreatic cancer, a colorectal cancer (e.g., a microsatellite stable colorectal cancer (MSS-CRC)), a lung cancer (e.g., a non-small cell lung cancer), a breast cancer (e.g., a triple negative breast cancer), a liver cancer (e.g., a hepatocellular carcinoma), a prostate cancer, or a renal cancer (e.g., a clear cell renal cell carcinoma).

### Exemplary VEGFR Inhibitors

In certain embodiments, the anti-CD73 antibody molecule described herein is administered in combination with a vascular endothelial growth factor (VEGF) receptor inhibitor (e.g., an inhibitor of one or more of VEGFR (e.g., VEGFR-1, VEGFR-2, or VEGFR-3) or VEGF). In some embodiments, the combination is used to treat a cancer, e.g., a cancer described herein, e.g., a solid tumor (e.g., a melanoma, a breast cancer, a colon cancer, an esophageal cancer, a gastrointestinal stromal tumor (GIST), a kidney cancer (e.g., a renal cell cancer), a liver cancer, a non-small cell lung cancer (NSCLC), an ovarian cancer, a pancreatic cancer, a prostate cancer, or a stomach cancer), e.g., a hematologic malignancy (e.g., a lymphoma).

In some embodiments, the VEGFR inhibitor is vatalanib succinate (Compound A47) or a compound disclosed in EP 296122.

In some embodiment, the VEGFR inhibitor is an inhibitor of one or more of VEGFR-2, PDGFRbeta, KIT or Raf kinase C, 1-methyl-5-((2-(5-(trifluoromethyl)-1H-imidazol-2-yl)pyridin-4-yl)oxy)-N-(4-(trifluoromethyl)phenyl)-1H-benzo[d]imidazol-2-amine (Compound A37) or a compound disclosed in PCT Publication No. WO 2007/030377.

Other exemplary VEGFR pathway inhibitors that can be used in the combinations disclosed herein include, e.g., bevacizumab (AVASTIN®), axitinib (INLYTA®); brivanib alaninate (BMS-582664, (S)-((R)-1-(4-(4-Fluoro-2-methyl-1H-indol-5-yloxy)-5-methylpyrrolo[2,1-f][1,2,4]triazin-6-yloxy)propan-2-yl)2-aminopropanoate); sorafenib (NEXAVAR®); pazopanib (VOTRIENT®); sunitinib malate (SUTENT®); cediranib (AZD2171, CAS 288383-20-1); vargatef (BIBF1120, CAS 928326-83-4); Foretinib (GSK1363089); telatinib (BAY57-9352, CAS 332012-40-5); apatinib (YN968D1, CAS 811803-05-1); imatinib (GLEEVEC®); ponatinib (AP24534, CAS 943319-70-8); tivozanib (AV951, CAS 475108-18-0); regorafenib (BAY73-4506, CAS 755037-03-7); vatalanib dihydrochloride (PTK787, CAS 212141-51-0); brivanib (BMS-540215, CAS 649735-46-6); vandetanib (CAPRELSA® or AZD6474); motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); linfanib (ABT869, CAS 796967-16-3); cabozantinib (XL184, CAS 849217-68-1); lestaurtinib (CAS 111358-88-4); N-[5-[[[5-(1,1-dimethylethyl)-2-oxazolyl]methyl]thio]-2-thiazolyl]-4-piperidinecarboxamide (BMS38703, CAS 345627-80-7); (3R,4R)-4-amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); 4-methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); aflibercept (EYLEA®), and endostatin (ENDOSTAR®).

Exemplary anti-VEGF antibodies that can be used in the combinations disclosed herein include, e.g., a monoclonal antibody that binds to the same epitope as the monoclonal anti-VEGF antibody A4.6.1 produced by hybridoma ATCC HB 10709; a recombinant humanized anti-VEGF monoclonal antibody generated according to Presta et al. (1997) Cancer Res. 57:4593-4599. In one embodiment, the anti-VEGF antibody is Bevacizumab (BV), also known as rhuMAb VEGF or AVASTIN®. It comprises mutated human IgG1 framework regions and antigen-binding complementarity-determining regions from the murine anti-hVEGF monoclonal antibody A.4.6.1 that blocks binding of human VEGF to its receptors. Bevacizumab and other humanized anti-VEGF antibodies are further described in U.S. Pat. No. 6,884,879 issued Feb. 26, 2005. Additional antibodies include the G6 or B20 series antibodies (e.g., G6-31, B20-4.1), as described in PCT Publication No. WO2005/012359, PCT Publication No. WO2005/044853, the contents of these patent applications are expressly incorporated herein by reference. For additional antibodies, see U.S. Pat. Nos. 7,060,269, 6,582,959, 6,703,020, 6,054,297, WO98/45332, WO 96/30046, WO94/10202, EP 0666868B1, U.S. Patent Application Publication Nos. 2006/009360, 2005/0186208, 2003/0206899, 2003/0190317, 2003/0203409, and 2005/0112126; and Popkov et al, Journal of Immunological Methods 288: 149-164 (2004). Other antibodies include those that bind to a functional epitope on human VEGF comprising of residues F17, M18, D19, Y21, Y25, Q89, 191, Kl 01, El 03, and C104 or, alternatively, comprising residues F17, Y21, Q22, Y25, D63, 183 and Q89.

Exemplary c-MET Inhibitors

In certain embodiments, the anti-CD73 antibody molecule described herein is administered in combination with an inhibitor of c-MET. In some embodiments, the combination is used to treat a cancer, e.g., a cancer described herein, e.g., a solid tumor (e.g., a non-small cell lung cancer, a pancreatic cancer, a liver cancer, a thyroid cancer (e.g., anaplastic thyroid carcinoma), a brain tumor (e.g., a glioblastoma), a kidney cancer (e.g., a renal cell carcinoma), or a head and neck cancer (e.g., a head and neck squamous cell carcinoma). In certain embodiments, the cancer is a liver cancer, e.g., a hepatocellular carcinoma (HCC) (e.g., a c-MET-expressing HCC).

In some embodiments, the c-MET inhibitor is Compound A17 or a compound described in U.S. Pat. Nos. 7,767,675 and 8,420,645).

In some embodiments, the c-MET inhibitor is JNJ-38877605. JNJ-38877605 is an orally available, small molecule inhibitor of c-Met. JNJ-38877605 selectively binds to c-MET, thereby inhibiting c-MET phosphorylation and disrupting c-Met signal transduction pathways.

In some embodiments, the c-Met inhibitor is AMG 208. AMG 208 is a selective small-molecule inhibitor of c-MET. AMG 208 inhibits the ligand-dependent and ligand-independent activation of c-MET, inhibiting its tyrosine kinase activity, which may result in cell growth inhibition in tumors that overexpress c-Met.

In some embodiments, the c-Met inhibitor is AMG 337. AMG 337 is an orally bioavailable inhibitor of c-Met. AMG 337 selectively binds to c-MET, thereby disrupting c-MET signal transduction pathways.

In some embodiments, the c-Met inhibitor is LY2801653. LY2801653 is an orally available, small molecule inhibitor of c-Met. LY2801653 selectively binds to c-MET, thereby inhibiting c-MET phosphorylation and disrupting c-Met signal transduction pathways.

In some embodiments, c-Met inhibitor is MSC2156119J. MSC2156119J is an orally bioavailable inhibitor of c-Met. MSC2156119J selectively binds to c-MET, which inhibits c-MET phosphorylation and disrupts c-Met-mediated signal transduction pathways.

In some embodiments, the c-MET inhibitor is capmatinib. Capmatinib is also known as INCB028060. Capmatinib is an orally bioavailable inhibitor of c-MET. Capmatinib selectively binds to c-Met, thereby inhibiting c-Met phosphorylation and disrupting c-Met signal transduction pathways.

In some embodiments, the c-MET inhibitor is crizotinib. Crizotinib is also known as PF-02341066. Crizotinib is an orally available aminopyridine-based inhibitor of the receptor tyrosine kinase anaplastic lymphoma kinase (ALK) and the c-Met/hepatocyte growth factor receptor (HGFR). Crizotinib, in an ATP-competitive manner, binds to and inhibits ALK kinase and ALK fusion proteins. In addition, crizotinib inhibits c-Met kinase, and disrupts the c-Met signaling pathway. Altogether, this agent inhibits tumor cell growth.

In some embodiments, the c-MET inhibitor is golvatinib. Golvatinib is an orally bioavailable dual kinase inhibitor of c-MET and VEGFR-2 with potential antineoplastic activity. Golvatinib binds to and inhibits the activities of both c-MET and VEGFR-2, which may inhibit tumor cell growth and survival of tumor cells that overexpress these receptor tyrosine kinases.

In some embodiments, the c-MET inhibitor is tivantinib. Tivantinib is also known as ARQ 197. Tivantinib is an orally bioavailable small molecule inhibitor of c-MET. Tivantinib binds to the c-MET protein and disrupts c-Met signal transduction pathways, which may induce cell death in tumor cells overexpressing c-MET protein or expressing constitutively activated c-Met protein.

### Exemplary IAP Inhibitors

In certain embodiments, the anti-CD73 antibody molecule described herein is administered in combination with an inhibitor of Inhibitor of Apoptosis Protein (IAP). In some embodiments, the combination is used to treat a cancer, e.g., a cancer described herein, e.g., a solid tumor (e.g., a colorectal cancer (CRC), a lung cancer (e.g., a non-small cell lung cancer (NSCLC)), a breast cancer (e.g., a triple negative breast cancer (TNBC)), an ovarian cancer, or a pancreatic cancer), e.g., a hematologic malignancy (e.g., a multiple myeloma).

In some embodiments, the IAP inhibitor is (S)—N—((S)-1-cyclohexyl-2-((S)-2-(4-(4-fluorobenzoyl)thiazol-2-yl)pyrrolidin-1-yl)-2-oxoethyl)-2-(methylamino)propanamide (Compound A21) or a compound disclosed in U.S. Pat. No. 8,552,003.

In some embodiments, the combination described herein includes an IAP inhibitor, (S)—N—((S)-1-cyclohexyl-2-((S)-2-(4-(4-fluorobenzoyl)thiazol-2-yl)pyrrolidin-1-yl)-2-oxoethyl)-2-(methylamino)propanamide (Compound A21), or a compound disclosed in U.S. Pat. No. 8,552,003, and an inhibitor of an immune checkpoint molecule, e.g., an inhibitor of CD73 (e.g., an anti-CD73 antibody molecule).

### Exemplary EGFR Inhibitors

In certain embodiments, the anti-CD73 antibody molecule described herein is administered in combination with an inhibitor of Epidermal Growth Factor Receptor (EGFR). In some embodiments, the combination is used to treat a cancer, e.g., a cancer described herein, e.g., a solid tumor (e.g., a lung cancer (e.g., a non-small cell lung cancer), a pancreatic cancer, a breast cancer (e.g., a triple negative breast cancer (TNBC)), or a colon cancer). In certain embodiments, the cancer is chosen from a colorectal cancer (e.g., a microsatellite stable colorectal cancer (MSS CRC)), a lung cancer (e.g., a non-small cell lung cancer), or a breast cancer (e.g., a triple negative lung cancer (TNBC)).

In some embodiments, the EGFR inhibitor is (R,E)-N-(7-chloro-1-(1-(4-(dimethylamino)but-2-enoyl)azepan-3-yl)-1H-benzo[d]imidazol-2-yl)-2-methylisonicotinamide (Compound A40) or a compound disclosed in PCT Publication No. WO 2013/184757.

In some embodiments, the combination described herein includes an EGFR inhibitor, (R,E)-N-(7-chloro-1-(1-(4-(dimethylamino)but-2-enoyl)azepan-3-yl)-1H-benzo[d]imidazol-2-yl)-2-methylisonicotinamide (Compound A40), or a compound disclosed in PCT Publication No. WO 2013/184757, and an inhibitor of an immune checkpoint molecule, e.g., an inhibitor of CD73 (e.g., an anti-CD73 antibody molecule).

In some embodiments, the EGFR inhibitor, (R,E)-N-(7-chloro-1-(1-(4-(dimethylamino)but-2-enoyl)azepan-3-yl)-1H-benzo[d]imidazol-2-yl)-2-methylisonicotinamide (Compound A40), or a compound disclosed in PCT Publication No. WO 2013/184757, is administered in combination with an inhibitor of CD73 (e.g., an anti-CD73 antibody molecule) to treat a colorectal cancer (CRC) (e.g., an MSS-CRC), a lung cancer (e.g., a non-small cell lung cancer (NSCLC)), or a breast cancer (e.g., a triple negative breast cancer (TNBC)).

In some embodiments, the EGFR inhibitor is chosen from one of more of erlotinib, gefitinib, cetuximab, panitumumab, necitumumab, PF-00299804, nimotuzumab, or R05083945.

Exemplary mTOR Inhibitors

In certain embodiments, the anti-CD73 antibody molecule described herein is administered in combination with an inhibitor of target of rapamycin (mTOR). In some embodiments, the combination is used to treat a cancer, e.g., a cancer described herein, e.g., a solid tumor (e.g., a prostate cancer, a breast cancer, a brain cancer, a bladder cancer, a pancreatic cancer, a renal cancer, or a liver cancer, a lung cancer (e.g., a small cell lung cancer or a non-small cell lung cancer), a respiratory/thoracic cancer, a sarcoma, a bone cancer, a non-small cell lung cancer, an endocrine cancer, an astrocytoma, a cervical cancer, a neurologic cancer, a gastric cancer, or a melanoma), e.g., a hematologic malignancy (e.g., a leukemia (e.g., lymphocytic leukemia), e.g., a lymphoma, or e.g., a multiple myeloma). In certain embodiments, the cancer is chosen from a colorectal cancer (e.g., a microsatellite stable colorectal cancer (MSS CRC)), a lung cancer (e.g., a non-small cell lung cancer), or a breast cancer (e.g., a triple negative lung cancer (TNBC)).

In some embodiments, the mTOR inhibitor is 8-(6-Methoxy-pyridin-3-yl)-3-methyl-1-(4-piperazin-1-yl-3-trifluoromethyl-phenyl)-1,3-dihydro-imidazo[4,5-c]quinolin-2-one (Compound A41).

In some embodiments, the mTOR inhibitor is everolimus (also known as AFINITOR®; Compound A36) or a compound disclosed in PCT Publication No. WO 2014/085318.

In some embodiments, the combination described herein includes the mTOR inhibitor, everolimus (Compound A36), or a compound disclosed in PCT Publication No. WO 2014/085318, and an inhibitor of an immune checkpoint molecule, e.g., an inhibitor of CD73 (e.g., an anti-CD73 antibody molecule).

In some embodiments, the mTOR inhibitor, Everolimus (Compound A36), or a compound disclosed in PCT Publication No. WO 2014/085318, is administered in combination with the CD73 inhibitor (e.g., the anti-CD73 antibody molecule) to treat a colorectal cancer, a lung cancer (e.g., a non-small cell lung cancer (NSCLC)), or a breast cancer (e.g., a triple negative breast cancer (NTBC)).

In some embodiments, the mTOR inhibitor is chosen from one or more of rapamycin, temsirolimus (TORISEL®), AZD8055, BGT226, XL765, PF-4691502, GDC0980, SF1126, OSI-027, GSK1059615, KU-0063794, WYE-354, Palomid 529 (P529), PF-04691502, or PKI-587. ridaforolimus (formally known as deferolimus, (1R,2R,4S)-4-[(2R)-2 [(1R,9S,12S,15R,16E,18R,19R,21R, 23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.04,9] hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (AFINITOR® or RAD001); rapamycin (AY22989, SIROLIMUS®); simapimod (CAS Registry Number: 164301-51-3); (5-12,4-Bis[(3S)-3-methylmorpholin-4-yl]pyrido[2,3-d]pyrimidin-7-yl1-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS Registry Number: 1013101-36-4); N2-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine (SEQ ID NO: 176) inner salt (SF1126, CAS Registry Number: 936487-67-1), or XL765 (SAR245409).

Other exemplary mTOR Inhibitors include, but are not limited to, temsirolimus; ridaforolimus (1R,2R,4S)-4-[(2R)-2 [(1R,9S,12S,15R,16E,18R,19R,21R, 23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}] hexatriaconta-16,24,26,28-tetraen-12-yl]propyl]-2-methoxycyclohexyl dimethylphosphinate, also known as AP23573 and MK8669; everolimus (RAD001); rapamycin (AY22989); simapimod; (5-{2,4-bis[(3S)-3-methylmorpholin-4-yl]pyrido[2,3-d]pyrimidin-7-yl}-2-methoxyphenyl)methanol (AZD8055); 2-amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine- (SEQ ID NO: 176), inner salt (SF1126); and XL765.

### Exemplary PI3K-γ, -δ Inhibitors

In certain embodiments, the anti-CD73 antibody molecule described herein is administered in combination with an inhibitor of phosphatidylinositol-4,5-bisphosphate 3-kinase (PI3K), e.g., phosphatidylinositol-4,5-bisphosphate 3-kinase gamma and/or delta (PI3K-y,6). In some embodiments, the combination is used to treat a cancer, e.g., a cancer described herein, e.g., a solid tumor (e.g., a prostate cancer, a breast cancer, a brain cancer, a bladder cancer, a pancreatic cancer, a renal cancer, a solid tumor, a liver cancer, a non-small cell lung cancer, an endocrine cancer, an ovarian cancer, a melanoma, a female reproductive system cancer, a digestive/gastrointestinal cancer, a glioblastoma multiforme, a head and neck cancer, or a colon cancer), e.g., a hematologic malignancy (e.g., a leukemia (e.g., a lymphocytic leukemia, e.g., chronic lymphocytic leukemia (CLL) (e.g., relapsed CLL)),e.g., a lymphoma (e.g., non-Hodgkin lymphoma (e.g., relapsed follicular B-cell non-Hodgkin lymphoma (FL) or relapsed small lymphocytic lymphoma (SLL)), or e.g., a multiple myeloma).

In some embodiments, the PI3K inhibitor is an inhibitor of delta and gamma isoforms of PI3K. Exemplary PI3K inhibitors that can be used in combination are described in, e.g., WO 2010/036380, WO 2010/006086, WO 09/114870, WO 05/113556, GSK 2126458, GDC-0980, GDC-0941, Sanofi XL147, XL756, XL147, PF-46915032, BKM 120, CAL-101, CAL 263, SF1126, PX-886, and a dual PI3K inhibitor.

In some embodiments, the PI3K-γ,δ inhibitor is idelalisib (CAS Registry Number: 870281-82-6). Idelalisib is also known as ZYDELIG®, GS-1101, CAL-101, or 5-Fluoro-3-phenyl-2-[(1S)-1-(7H-purin-6-ylamino)propyl]-4(3H)-quinazolinone. Idelalisib blocks P1106, the delta isoform of PI3K. Idelalisib is disclosed, e.g., in Wu et al. Journal of Hematology & Oncology (2013) 6: 36.

In some embodiments, the PI3K-γ,δ inhibitor is 8-(6-Methoxy-pyridin-3-yl)-3-methyl-1-(4-piperazin-1-yl-3-trifluoromethyl-phenyl)-1,3-dihydro-imidazo[4,5-c]quinolin-2-one (Compound A41).

In some embodiments, the PI3K-γ,δ inhibitor is buparlisib (Compound A6) or a compound disclosed in PCT Publication No. WO 2007/084786.

Other exemplary PI3K-γ,δ inhibitors that can be used in the combination include, e.g., pictilisib (GDC-0941), LY294002, pilaralisib (XL147), PI-3065, PI-103, VS-5584 (SB2343), CZC24832, duvelisib (IPI-145, INK1197), TG100-115, CAY10505, GSK1059615, PF-04691502, AS-605240, voxtalisib (SAR245409, XL765), IC-87114, omipalisib (GSK2126458, GSK458), TG100713, gedatolisib (PF-05212384, PKI-587), PKI-402, XL147 analogue, PIK-90, PIK-293, PIK-294, 3-Methyladenine (3-MA), AS-252424, AS-604850, or apitolisib (GDC-0980, RG7422).

In some embodiments, the PI3K inhibitor is Compound A8 or a compound described in PCT Publication No. WO2010/029082.

In some embodiments, the PI3K inhibitor is a pan-PI3K inhibitor, (4S,5R)-3-(2′-amino-2-morpholino-4′-(trifluoromethyl)-[4,5′-bipyrimidin]-6-yl)-4-(hydroxymethyl)-5-methyloxazolidin-2-one (Compound A13) or a compound disclosed in PCT Publication No. WO2013/124826.

Exemplary PI3K-γ, -δ inhibitors include, but are not limited to, duvelisib and idelalisib. Idelalisib (also called GS-1101 or CAL-101; Gilead) is a small molecule that blocks the delta isoform of PI3K. The structure of idelalisib (5-Fluoro-3-phenyl-2-[(1S)-1-(7H-purin-6-ylamino)propyl]-4(3H)-quinazolinone) is shown below.

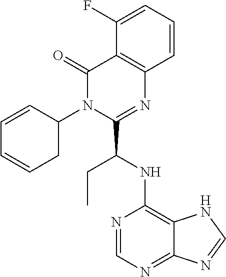 [SMILES: CC[C@H](Nc1ncnc2nc[nH]c12)c1nc2cccc(F)c2c(=O)n1C1C=CC=CC1]

Duvelisib (also called IPI-145; Infinity Pharmaceuticals and Abbvie) is a small molecule that blocks PI3K-δ,γ. The structure of duvelisib (8-Chloro-2-phenyl-3-[(1S)-1-(9H-purin-6-ylamino)ethyl]-1(2H)-isoquinolinone) is shown below.

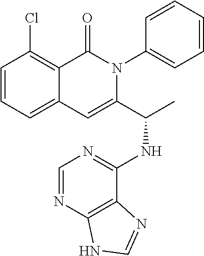 [SMILES: C[C@H](Nc1ncnc2[nH]cnc12)c1cc2cccc(Cl)c2c(=O)n1-c1ccccc1]

In one embodiment, the inhibitor is a dual phosphatidylinositol 3-kinase (PI3K) and mTOR inhibitor selected from 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF-04691502); N-[4-[[4-(Dimethylamino)-1-piperidinyl]carbonyl]phenyl]-N-[4-(4,6-di-4-morpholinyl-1,3,5-triazin-2-yl)phenyl]urea (PF-05212384, PKI-587); apitolisib (GDC-0980, RG7422); 2,4-Difluoro-N-{2-(methyloxy)-5-[4-(4-pyridazinyl)-6-quinolinyl]-3-pyridinyl}benzenesulfonamide (GSK2126458); 8-(6-methoxypyridin-3-yl)-3-methyl-1-(4-(piperazin-1-yl)-3-(trifluoromethyl)phenyl)-1H-imidazo[4,5-c] quinolin-2(3H)-one Maleic acid (NVP-BGT226); 3-[4-(4-Morpholinylpyrido[3′,2′:4,5]furo[3,2-d]pyrimidin-2-yl]phenol (PI-103); 5-(9-isopropyl-8-methyl-2-morpholino-9H-purin-6-yl)pyrimidin-2-amine (VS-5584, SB2343); or N-[2-[(3,5-Dimethoxyphenyl)amino]quinoxalin-3-yl]-[4(4-methyl-3-methoxyphenyl)carbonyl]aminophenylsulfonamide (XL765).

### Exemplary JAK Inhibitors

In certain embodiments, the anti-CD73 antibody molecule described herein is administered in combination with an inhibitor of Janus kinase (JAK). In some embodiments, the combination is used to treat a cancer, e.g., a cancer described herein, e.g., a solid tumor (e.g., a colon cancer, a prostate cancer, a lung cancer, a breast cancer, or a pancreatic cancer), e.g., a hematologic malignancy (e.g., a leukemia (e.g., a myeloid leukemia or a lymphocytic leukemia), e.g., a lymphoma (e.g., a non-Hodgkin lymphoma), or a multiple myeloma.

In some embodiments, the JAK inhibitor is 2-fluoro-N-methyl-4-(7-(quinolin-6-ylmethyl)imidazo[1,2-b][1,2,4]triazin-2-yl)benzamide (Compound A17), or a dihydrochloric salt thereof, or a compound disclosed in PCT Publication No. WO 2007/070514.

In some embodiment, the JAK inhibitor is ruxolitinib phosphate (also known as JAKAFI; Compound A18) or a compound disclosed in PCT Publication No. WO 2007/070514.

### Exemplary Cell Therapies

Anti-CD73 antibody molecules can also be combined with a cell therapy, e.g., a chimeric antigen receptor (CAR) therapy, a T cell therapy, a natural killer (NK) cell therapy, or a dendritic cell therapy.

Combinations with CAR Therapies

The anti-CD73 antibody molecules described herein can be administered in combination with a second therapeutic, e.g., a cell comprising a chimeric antigen receptor (CAR). The CAR may comprise i) an extracellular antigen binding domain, ii) a transmembrane domain, and iii) an intracellular signaling domain (which may comprise one or both of a primary signaling domain and a costimulatory domain). The CAR may further comprise a leader sequence and/or a hinge sequence. In specific embodiments, the CAR construct comprises a scFv domain, wherein the scFv may be preceded by an optional leader sequence, and followed by an optional hinge sequence, a transmembrane region, and an intracellular signaling domain, e.g., wherein the domains are contiguous with and in the same reading frame to form a single fusion protein.

In some embodiments, the CAR molecule comprises a CD19 CAR molecule described herein, e.g., a CD19 CAR molecule described in US 2015/0283178, e.g., CTL019. In embodiments, the CD19 CAR comprises an amino acid, or has a nucleotide sequence shown in US 2015/0283178, incorporated herein by reference in its entirety, or a sequence substantially identical thereto (e.g., a sequence having at least about 85%, 90%, or 95% sequence identity thereto).

In one embodiment, the CAR T cell that binds to CD19 has the USAN designation TISAGENLECLEUCEL-T. CTL019 is made by a gene modification of T cells mediated by stable insertion via transduction with a self-inactivating, replication deficient lentiviral (LV) vector containing the CTL019 transgene under the control of the EF-1 alpha promoter. CTL019 can be a mixture of transgene positive and negative T cells that are delivered to the subject on the basis of percent transgene positive T cells.

In one embodiment, the CD19 CAR comprises an amino acid sequence provided as SEQ ID NO: 12 in PCT publication WO2012/079000. In embodiment, the amino acid sequence is:

(SEQ ID NO: 132)
MALPVTALLLPLALLLHAARPdiqmtqttsslsaslgdrvtiscrasqdi
skylnwyqqkpdgtvklliyhtsrlhsgvpsrfsgsgsgtdysitisnle
qediatyfcqqgntlpytfgggtkleitggggsggggsggggsevklqes
gpglvapsqs1svtctvsgvslpdygvswirqpprkglewlgviwgsett
yynsalksrltiikdnsksqvflkmnslqtddtaiyycakhyyyggsyam
dywgqgtsvtvsstapaprpptpaptiasqp1s1rpeacrpaaggavhtr
gldfacdiyiwaplagtcgvillslvitlyckrgrkkllyiflcqpfmrp
vqttqeedgcscrfpeeeeggcelrvkfsrsadapaykqgqnqlynelnl
grreeydvldkrrgrdpemggkprrknpqeglynelqkdkmaeayseigm
kgerrrgkghdglyqglstatkdtydalhmqalppr,

or a sequence substantially identical thereto (e.g., a sequence having at least about 85%, 90%, or 95% sequence identity thereto), with or without the signal peptide sequence indicated in capital letters.

In one embodiment, the amino acid sequence is:

(SEQ ID NO: 133)
diqmtqttsslsaslgdrvtiscrasqdiskylnwyqqkpdgtvklliyh
tsrlhsgvpsrfsgsgsgtdysltisnleqediatyfcqqgntlpytfgg
gtkleitggggsggggsggggsevklqesgpglvapsqs1svtctvsgvs
lpdygvswirqpprkglewlgviwgsettyynsalksritiikdnsksqv
flkmnslqtddtaiyycakhyyyggsyamdywgqgtsvtvsstapaprpp
tpaptiasqp1s1rpeacrpaaggavhtrgldfacdiyiwaplagtcgvi
llslvitlycicrgrkkllyiflcqpfmrpvqttqeedgcscrfpeeeeg
gcelrvkfsrsadapaykqgqnqlynelnlgrreeydvldkrrgrdpemg
gkprrknpqeglynelqkdkmaeayseigmkgeragkghdglyqglstat
kdtydalhmqalppr,

or a sequence substantially homologous thereto (e.g., a sequence having at least about 85%, 90%, or 95% sequence identity thereto).

### Antigen Binding Domain of a Chimeric Antigen Receptor (CAR)

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment.

In some embodiments, the antigen binding domain of the CAR is a scFv antibody fragment that is humanized compared to the murine sequence of the scFv from which it is derived.

In some embodiments, the antigen binding domain binds a tumor antigen described herein. In embodiments, the tumor antigen is chosen from: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDG1cp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDG1cp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAXS); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRLS); and immunoglobulin lambda-like polypeptide 1 (IGLL1).

In one embodiment, the CAR molecule comprises a BCMA CAR molecule, e.g., a BCMA CAR described in US 2016/0046724 or WO 2016/014565, incorporated herein by reference. In embodiments, the BCMA CAR comprises an amino acid, or has a nucleotide sequence of a CAR molecule, or an antigen binding domain according to US 2016/0046724, or Table 1 or 16, SEQ ID NO: 271 or SEQ ID NO: 273 of WO 2016/014565, incorporated herein by reference, or a sequence substantially identical to any of the aforesaid sequences (e.g., having at least about 85%, 90%, or 95% sequence identity to any of the aforesaid BCMA CAR sequences). The amino acid and nucleotide sequences encoding the BCMA CAR molecules and antigen binding domains (e.g., including one, two, three VH CDRs; and one, two, three VL CDRs according to Kabat or Chothia), are specified in WO 2016/014565.

### Transmembrane Domain of a Chimeric Antigen Receptor (CAR)

With respect to the transmembrane domain, in various embodiments, a CAR can be designed to comprise a transmembrane domain that is attached to the extracellular domain of the CAR.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the CAR has bound to a target. A transmembrane domain may include at least the transmembrane region(s) of e.g., the alpha, beta or zeta chain of the T-cell receptor, CD28, CD27, CD3 epsilon, CD45, CD4, CDS, CD8, CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, or NKG2C.

In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge (e.g., an IgG4 hinge, an IgD hinge), a GS linker (e.g., a GS linker described herein), a KIR2DS2 hinge or a CD8a hinge.

### Intracellular Signaling Domain of a Chimeric Antigen Receptor (CAR)

The cytoplasmic domain or region of the CAR includes an intracellular signaling domain. An intracellular signaling domain is generally responsible for activation of at least one of the normal effector functions of the immune cell in which the CAR has been introduced.

Examples of intracellular signaling domains for use in the CAR include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains include those of CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12. In one embodiment, a CAR comprises an intracellular signaling domain, e.g., a primary signaling domain of CD3-zeta.

The intracellular signaling domain of the CAR can comprise the CD3-zeta signaling domain by itself or it can be combined with any other desired intracellular signaling domain(s) useful in the context of a CAR of the invention. For example, the intracellular signaling domain of the CAR can comprise a CD3 zeta chain portion and a costimulatory signaling domain. The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that binds to CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a.

### Activation and Expansion of Immune Effector Cells (e.g., T Cells)

Immune effector cells such as T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 2006/0121005, incorporated herein by reference.

Examples of immune effector cells include T cells, e.g., alpha/beta T cells and gamma/delta T cells, B cells, natural killer (NK) cells, natural killer T (NKT) cells, mast cells, and myeloid-derived phagocytes.

Methods of making CAR-expressing cells are described, e.g., in US 2016/0185861, incorporated herein by reference.

### Exemplary Cancer Vaccines

Anti-CD73 antibody molecules can be combined with an immunogenic agent, such as cancerous cells, purified tumor antigens (including recombinant proteins, peptides, and carbohydrate molecules), cells, and cells transfected with genes encoding immune stimulating cytokines (He et al. (2004) J. Immunol. 173:4919-28). Non-limiting examples of tumor vaccines that can be used include peptides of melanoma antigens, such as peptides of gp100, MAGE antigens, Trp-2, MART1 and/or tyrosinase, tumor cells transfected to express the cytokine GM-CSF, DNA-based vaccines, RNA-based vaccines, and viral transduction-based vaccines. The cancer vaccine may be prophylactic or therapeutic.

CD73 blockade can be used in conjunction with a collection of recombinant proteins and/or peptides expressed in a tumor in order to generate an immune response to these proteins. Other tumor vaccines may include the proteins from viruses implicated in human cancers such a

Human Papilloma Viruses (HPV), Hepatitis Viruses (HBV and HCV), Kaposi's Herpes Sarcoma Virus (KHSV), and Epstein-Barr virus (EBV). Another form of tumor specific antigen which may be used in conjunction with CD73 blockade is purified heat shock proteins (HSP) isolated from the tumor tissue itself. These heat shock proteins contain fragments of proteins from the tumor cells and these HSPs are highly efficient at delivery to antigen presenting cells for eliciting tumor immunity (Suot, R & Srivastava, P (1995) Science 269:1585-1588; Tamura, Y. et al. (1997) Science 278:117-120).

Dendritic cells (DC) are potent antigen presenting cells that can be used to prime antigen-specific responses. DC's can be produced ex vivo and loaded with various protein and peptide antigens as well as tumor cell extracts (Nestle, F. et al. (1998) Nature Medicine 4: 328-332). DCs may also be transduced by genetic means to express these tumor antigens as well. DCs have also been fused directly to tumor cells for the purposes of immunization (Kugler, A. et al. (2000) Nature Medicine 6:332-336). As a method of vaccination, DC immunization may be effectively combined with CD73 blockade to activate more potent anti-tumor responses.

### Exemplary Oncolytic Viruses

Anti-CD73 antibody molecules can be administered in combination with oncolytic viruses. In embodiments, oncolytic viruses are capable of selectively replicating in and triggering the death of or slowing the growth of a cancer cell. In some cases, oncolytic viruses have no effect or a minimal effect on non-cancer cells. In some embodiments, the combination is used to treat a cancer, e.g., a cancer described herein. In certain embodiments, the cancer is a brain cancer, e.g., a glioblastoma (GBM). An oncolytic virus includes, but is not limited to, an oncolytic adenovirus, oncolytic Herpes Simplex Viruses, oncolytic retrovirus, oncolytic parvovirus, oncolytic vaccinia virus, oncolytic Sindbis virus, oncolytic influenza virus, or oncolytic RNA virus (e.g., oncolytic reovirus, oncolytic Newcastle Disease Virus (NDV), oncolytic measles virus, or oncolytic vesicular stomatitis virus (VSV)).

Exemplary oncolytic viruses include but are not limited to the following:

Group B Oncolytic Adenovirus (ColoAdl) (PsiOxus Therapeutics Ltd.) (see, e.g., Clinical Trial Identifier: NCT02053220);

ONCOS-102 (previously called CGTG-102), which is an adenovirus comprising granulocyte-macrophage colony stimulating factor (GM-CSF) (Oncos Therapeutics) (see, e.g., Clinical Trial Identifier: NCT01598129);

VCN-01, which is a genetically modified oncolytic human adenovirus encoding human PH2O hyaluronidase (VCN Biosciences, S.L.) (see, e.g., Clinical Trial Identifiers: NCT02045602 and NCT02045589);

Conditionally Replicative Adenovirus ICOVIR-5, which is a virus derived from wild-type human adenovirus serotype 5 (Had5) that has been modified to selectively replicate in cancer cells with a deregulated retinoblastoma/E2F pathway (Institut Catala d'Oncologia) (see, e.g., Clinical Trial Identifier: NCT01864759);

Celyvir, which comprises bone marrow-derived autologous mesenchymal stem cells (MSCs) infected with ICOVIR5, an oncolytic adenovirus (Hospital Infantil Universitario Niño Jesús, Madrid, Spain/Ramon Alemany) (see, e.g., Clinical Trial Identifier: NCT01844661);

CG0070, which is a conditionally replicating oncolytic serotype 5 adenovirus (Ad5) in which human E2F-1 promoter drives expression of the essential Ela viral genes, thereby restricting viral replication and cytotoxicity to Rb pathway-defective tumor cells (Cold Genesys, Inc.) (see, e.g., Clinical Trial Identifier: NCT02143804); or

DNX-2401 (formerly named Delta-24-RGD), which is an adenovirus that has been engineered to replicate selectively in retinoblastoma (Rb)-pathway deficient cells and to infect cells that express certain RGD-binding integrins more efficiently (Clinica Universidad de Navarra, Universidad de Navarra/DNAtrix, Inc.) (see, e.g., Clinical Trial Identifier: NCT01956734).

In some embodiments, an oncolytic virus described herein is administering by injection, e.g., subcutaneous, intra-arterial, intravenous, intramuscular, intrathecal, or intraperitoneal injection. In embodiments, an oncolytic virus described herein is administered intratumorally, transdermally, transmucosally, orally, intranasally, or via pulmonary administration.

### Additional Exemplary Cancer Therapies

Exemplary combinations of anti-CD73 antibody molecules (alone or in combination with other stimulatory agents) and standard of care for cancer, include at least the following. In certain embodiments, the anti-CD73 antibody molecule, e.g., the anti-CD73 antibody molecule described herein, is used in combination with a standard of cancer care chemotherapeutic agent including, but not limited to, anastrozole (Arimidex®), bicalutamide (Casodex®), bleomycin sulfate (Blenoxane®), busulfan (Myleran®), busulfan injection (Busulfex), capecitabine (Xeloda), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome), dexamethasone, docetaxel (Taxotere®), doxorubicin hydrochloride (Adriamycin®, Rubex®), etoposide (Vepesid), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), vinorelbine (Navelbine®), Ibrutinib, idelalisib, and brentuximab vedotin.

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin. Exemplary vinca alkaloids that can be used in combination with the anti-CD73 antibody molecules, include, but are not limited to, vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteosome inhibitors that can be used in combination with the anti-CD73 antibody molecules include, but are not limited to, bortezomib (Velcade®); carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); and O-Methyl-N-[2-methyl-5-thiazolyl)carbonyl]-L-seryl-O-methyl-N—[1S)-2-[(2R)-2-methyl-2-oxiranyl]-2-oxo-1-(phenylmethyl)ethyl]-L-serinamide (ONX-0912).

In some embodiments, the anti-CD73 antibody molecule, e.g., the anti-CD73 antibody molecule described herein, is used, in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-B inhibitor)), a RAF-1 inhibitor, a KIT inhibitor and a RET inhibitor. In some embodiments, the anti-cancer agent used in combination with the hedgehog inhibitor is selected from the group consisting of: axitinib (AG013736), bosutinib (SKI-606), cediranib (RECENTIN™, AZD2171), dasatinib (SPRYCELO, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951(tivozanib), axitinib, BAY 73-4506 (regorafenib), brivanib alaninate (BMS-582664), brivanib (BMS-540215), cediranib (AZD2171), CP 673451, CYC116, E7080, Ki8751, masitinib (AB1010), MGCD-265, motesanib diphosphate (AMG-706), MP-470, OSI-930, Pazopanib Hydrochloride, PD173074, Sorafenib Tosylate(Bay 43-9006), SU 5402, TSU-68(SU6668), vatalanib, XL880 (GSK1363089, EXEL-2880). Selected tyrosine kinase inhibitors are chosen from sunitinib, erlotinib, gefitinib, or sorafenib.

Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” refers to radiation therapy delivered by a spatially confined radioactive material inserted into the body at or near a tumor or other proliferative tissue disease site. The term is intended without limitation to include exposure to radioactive isotopes (e.g., At-211, 1-131, 1-125, Y-90, Re-186, Re-188, Sm-153, Bi-212, P-32, and radioactive isotopes of Lu). Suitable radiation sources for use as a cell conditioner of the present invention include both solids and liquids. By way of non-limiting example, the radiation source can be a radionuclide, such as 1-125, 1-131, Yb-169, Ir-192 as a solid source, 1-125 as a solid source, or other radionuclides that emit photons, beta particles, gamma radiation, or other therapeutic rays. The radioactive material can also be a fluid made from any solution of radionuclide(s), e.g., a solution of 1-125 or 1-131, or a radioactive fluid can be produced using a slurry of a suitable fluid containing small particles of solid radionuclides, such as Au-198, Y-90.

CD73 blockade may also be effectively combined with chemotherapeutic regimes. In these instances, it may be possible to reduce the dose of chemotherapeutic reagent administered.

Exemplary cytotoxic agents that can be administered in combination with an anti-CD73 antibody molecule include antimicrotubule agents, topoisomerase inhibitors, anti-metabolites, mitotic inhibitors, alkylating agents, anthracyclines, vinca alkaloids, intercalating agents, agents capable of interfering with a signal transduction pathway, agents that promote apoptosis, proteosome inhibitors, and radiation (e.g., local or whole body irradiation).

In certain embodiments, any of the combinations disclosed herein, alternatively or in combination, further includes one or more of the agents described in Table 18.

TABLE 18
Selected therapeutic agents that can be administered in combination with the anti-CD73 antibody molecules, e.g., as a single
agent or in combination with other immunomodulators described herein. Each publication listed in this Table is herein incorporated
by reference in its entirety, including all structural formulae therein.

| | Generic | | Patents/Patent |
|---|---|---|---|
| Compound | Name | | Application |
| Designation | Tradename | Compound Structure | Publications |
| A1 | Sotrastaurin | 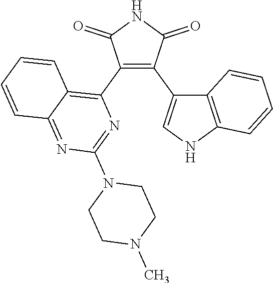 [SMILES: CN1CCN(c2nc(C3=C(c4c[nH]c5ccccc45)C(=O)NC3=O)c3ccccc3n2)CC1] | EP 1682103 US 2007/142401 WO 2005/039549 |
| A2 | Nilotinib HCl monohydrate TASIGNA ® | 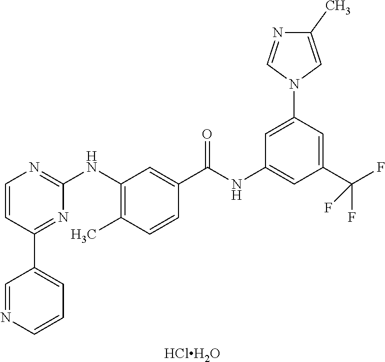 [SMILES: Cc1cn(-c2cc(NC(=O)c3ccc(C)c(Nc4nccc(-c5cccnc5)n4)c3)cc(C(F)(F)F)c2)cn1 ; "HCl·H2O"] | WO 2004/005281 U.S. Pat. No. 7,169,791 |
| A7 | | 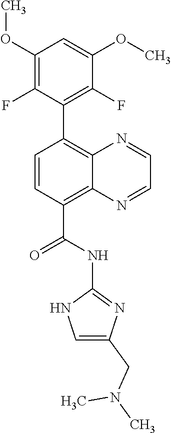 [SMILES: COc1cc(OC)c(F)c(-c2ccc(C(=O)Nc3nc(CN(C)C)c[nH]3)c3nccnc23)c1F] | WO 2009/141386 US 2010/0105667 |
| A8 | | 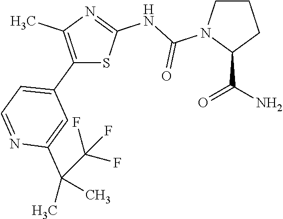 [SMILES: Cc1nc(NC(=O)N2CCC[C@H]2C(N)=O)sc1-c1ccnc(C(C)(C)C(F)(F)F)c1] | WO 2010/029082 |
| A10 | | 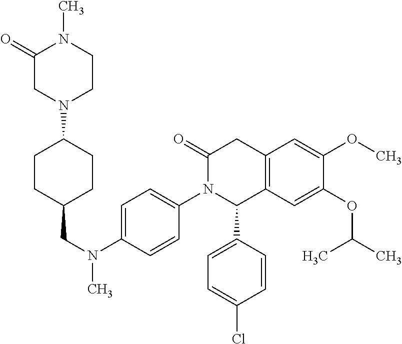 [SMILES: COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2] | WO 2011/076786 |
| A11 | Deferasirox EXJADE ® | 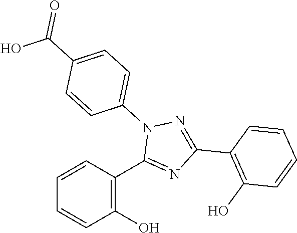 [SMILES: O=C(O)c1ccc(-n2nc(-c3ccccc3O)nc2-c2ccccc2O)cc1] | WO 1997/049395 |
| A12 | Letrozole FEMARA ® | 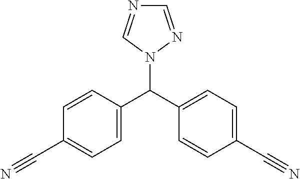 [SMILES: N#Cc1ccc(C(c2ccc(C#N)cc2)n2cncn2)cc1] | U.S. Pat. No. 4,976,672 |
| A13 | | 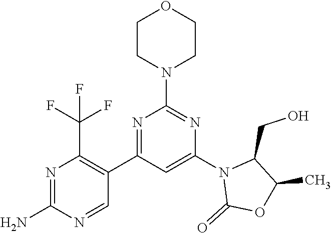 [SMILES: C[C@H]1OC(=O)N(c2cc(-c3cnc(N)nc3C(F)(F)F)nc(N3CCOCC3)n2)[C@H]1CO] | WO 2013/124826 US 2013/0225574 |
| A14 | | 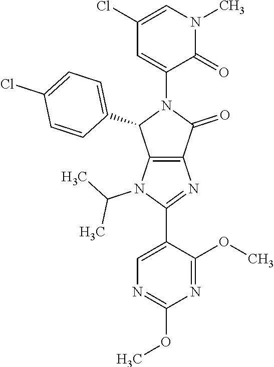 [SMILES: COc1ncc(-c2nc3c(n2C(C)C)[C@H](c2ccc(Cl)cc2)N(c2cc(Cl)cn(C)c2=O)C3=O)c(OC)n1] | WO 2013/111105 |
| A15 | | 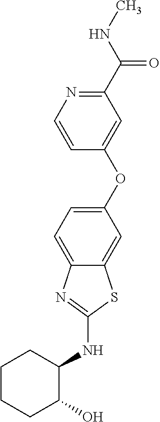 [SMILES: CNC(=O)c1cc(Oc2ccc3nc(N[C@@H]4CCCC[C@H]4O)sc3c2)ccn1] | WO 2005/073224 |
| A16 | Imatinib mesylate GLEEVEC ® | 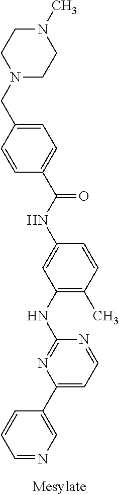 [SMILES: Cc1ccc(NC(=O)c2ccc(CN3CCN(C)CC3)cc2)cc1Nc1nccc(-c2cccnc2)n1] | WO 1999/003854 |
| A17 | | 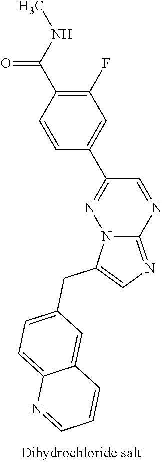 [SMILES: CNC(=O)c1ccc(-c2cnc3ncc(Cc4ccc5ncccc5c4)n3n2)cc1F] | EP 2099447 U.S. Pat. No. 7,767,675 U.S. Pat. No. 8,420,645 |
| A18 | Ruxolitinib Phosphate JAKAFI ® | 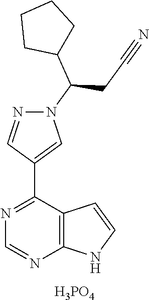 | WO 2007/070514 EP 2474545 U.S. Pat. No. 7,598,257 WO 2014/018632 |
| A19 | Panobinostat | 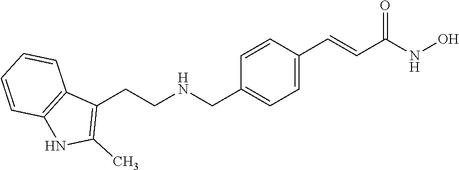 [SMILES: Cc1[nH]c2ccccc2c1CCNCc1ccc(/C=C/C(=O)NO)cc1] | WO 2014/072493 WO 2002/022577 EP 1870399 |
| A20 | Osilodrostat | 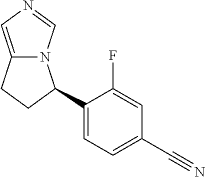 [SMILES: N#Cc1ccc([C@H]2CCc3cncn32)c(F)c1] | WO 2007/024945 |
| A21 | | 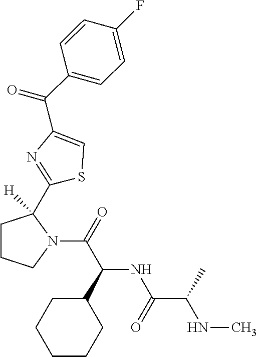 [SMILES: CN[C@@H](C)C(=O)N[C@H](C(=O)N1CCC[C@H]1c1nc(C(=O)c2ccc(F)cc2)cs1)C1CCCCC1] | WO 2008/016893 EP 2051990 U.S. Pat. No. 8,546,336 |
| A23 | ceritinib ZYKADIA ™ | 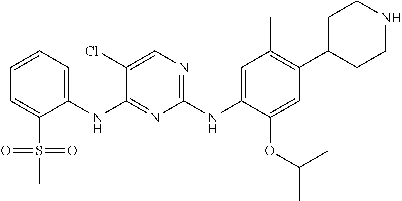 [SMILES: Cc1cc(Nc2ncc(Cl)c(Nc3ccccc3S(C)(=O)=O)n2)c(OC(C)C)cc1C1CCNCC1] | WO 2008/073687 U.S. Pat. No. 8,039,479 |
| A24 | | 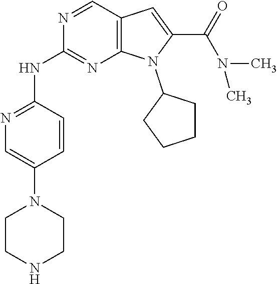 [SMILES: CN(C)C(=O)c1cc2cnc(Nc3ccc(N4CCNCC4)cn3)nc2n1C1CCCC1] | U.S. Pat. No. 8,415,355 U.S. Pat. No. 8,685,980 |
| A25 | | 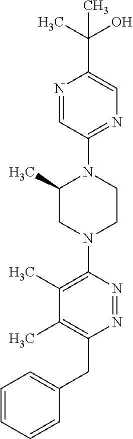 [SMILES: Cc1c(Cc2ccccc2)nnc(N2CCN(c3cnc(C(C)(C)O)cn3)[C@H](C)C2)c1C] | WO 2010/007120 |
| A26 | | Human monoclonal antibody to PRLR | U.S. Pat. No. 7,867,493 |
| A27 | | 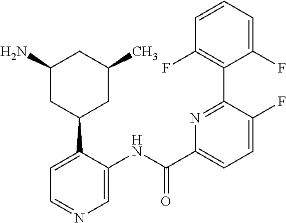 [SMILES: C[C@@H]1C[C@H](N)C[C@H](c2ccncc2NC(=O)c2ccc(F)c(-c3c(F)cccc3F)n2)C1] | WO 2010/026124 EP 2344474 US 2010/0056576 WO 2008/106692 |
| A28 | | 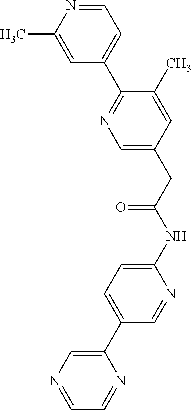 [SMILES: Cc1cc(-c2ncc(CC(=O)Nc3ccc(-c4cnccn4)cn3)cc2C)ccn1] | WO 2010/101849 |
| A30 | | 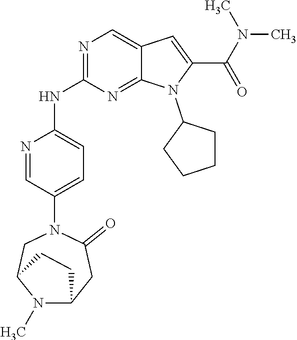 [SMILES: CN(C)C(=O)c1cc2cnc(Nc3ccc(N4C[C@H]5CC[C@@H](CC4=O)N5C)cn3)nc2n1C1CCCC1] | WO 2011/101409 |
| A31 | | Human monoclonal antibody to HER3 | WO 2012/022814 |
| | | | EP 2606070 |
| | | | U.S. Pat. No. 8,735,551 |
| A32 | | Antibody Drug Conjugate (ADC) | WO 2014/160160 |
| | | | Ab: 12425 (see |
| | | | Table 1, paragraph |
| | | | [00191]) |
| | | | Linker: SMCC (see |
| | | | paragraph [00117] |
| | | | Payload: DM1 (see |
| | | | paragraph [00111] |
| | | | See also Claim 29 |
| A33 | | Monoclonal antibody or Fab to M-CSF | WO 2004/045532 |
| A35 | Midostaurin | 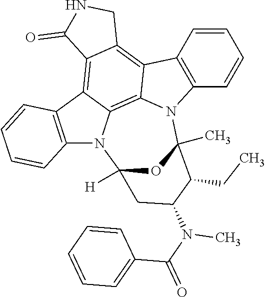 [SMILES: CC[C@@H]1[C@H](N(C)C(=O)c2ccccc2)C[C@H]2O[C@]1(C)n1c3ccccc3c3c4c(c5c6ccccc6n2c5c31)C(=O)NC4] | WO 2003/037347 EP 1441737 US 2012/252785 |
| A36 | Everolimus AFINITOR ® | 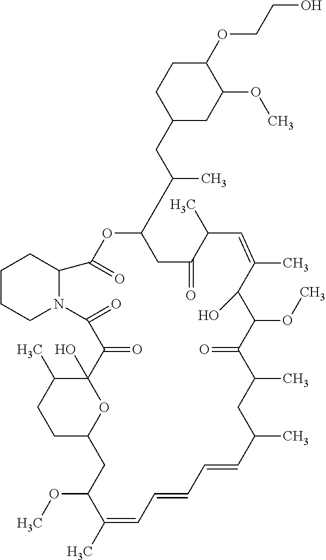 [SMILES: COC1CC2CCC(C)C(O)(O2)C(=O)C(=O)N2CCCCC2C(=O)OC(C(C)CC2CCC(OCCO)C(OC)C2)CC(=O)C(C)/C=C(/C)C(O)C(OC)C(=O)C(C)CC(C)/C=C/C=C/C=C\1C] | WO 2014/085318 |
| A37 | | 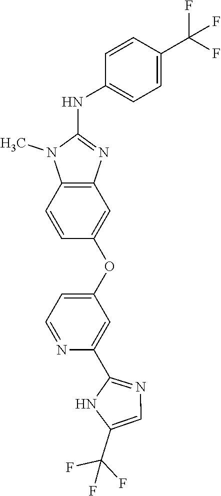 [SMILES: Cn1c(Nc2ccc(C(F)(F)F)cc2)nc2cc(Oc3ccnc(-c4ncc(C(F)(F)F)[nH]4)c3)ccc21] | WO 2007/030377 U.S. Pat. No. 7,482,367 |
| A38 | Pasireotide diaspartate SIGNIFOR ® | 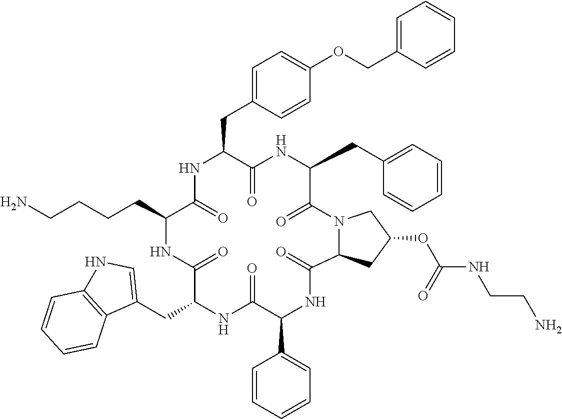 [SMILES: NCCCC[C@@H]1NC(=O)[C@@H](Cc2c[nH]c3ccccc23)NC(=O)[C@H](c2ccccc2)NC(=O)[C@@H]2C[C@@H](OC(=O)NCCN)CN2C(=O)[C@H](Cc2ccccc2)NC(=O)[C@H](Cc2ccc(OCc3ccccc3)cc2)NC1=O] | WO 2002/010192 U.S. Pat. No. 7,473,761 |
| A40 | | 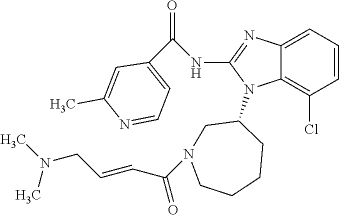 [SMILES: Cc1cc(C(=O)Nc2nc3cccc(Cl)c3n2[C@@H]2CCCCN(C(=O)/C=C/CN(C)C)C2)ccn1] | WO 2013/184757 |
| A41 | | 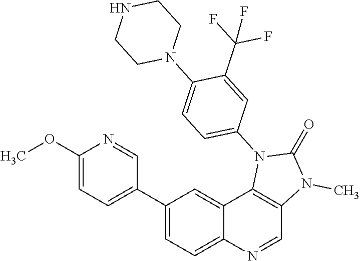 | WO 2006/122806 |
| A42 | | 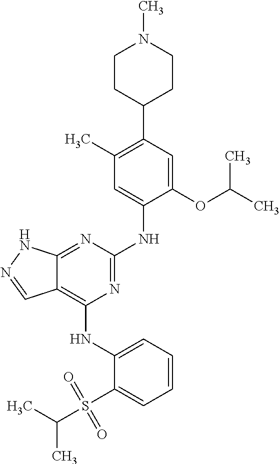 [SMILES: Cc1cc(Nc2nc(Nc3ccccc3S(=O)(=O)C(C)C)c3cn[nH]c3n2)c(OC(C)C)cc1C1CCN(C)CC1] | WO 2008/073687 U.S. Pat. No. 8,372,858 |
| A43 | | 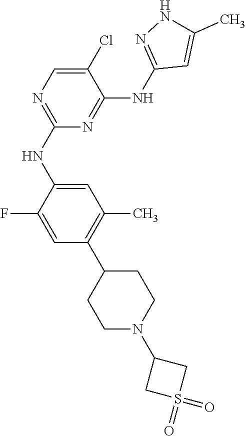 [SMILES: Cc1cc(Nc2nc(Nc3cc(C)c(C4CCN(C5CS(=O)(=O)C5)CC4)cc3F)ncc2Cl)n[nH]1] | WO 2010/002655 U.S. Pat. No. 8,519,129 |
| A44 | | 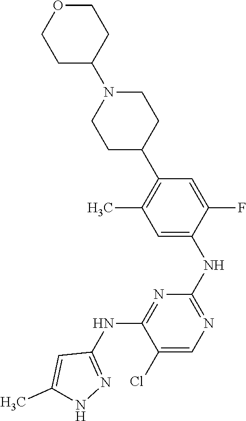 [SMILES: Cc1cc(Nc2nc(Nc3cc(C)c(C4CCN(C5CCOCC5)CC4)cc3F)ncc2Cl)n[nH]1] | WO 2010/002655 U.S. Pat. No. 8,519,129 |
| A45 | | 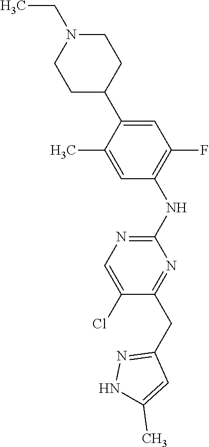 [SMILES: CCN1CCC(c2cc(F)c(Nc3ncc(Cl)c(Cc4cc(C)[nH]n4)n3)cc2C)CC1] | WO 2010/002655 |
| A46 | Valspodar AMDRAY ™ | 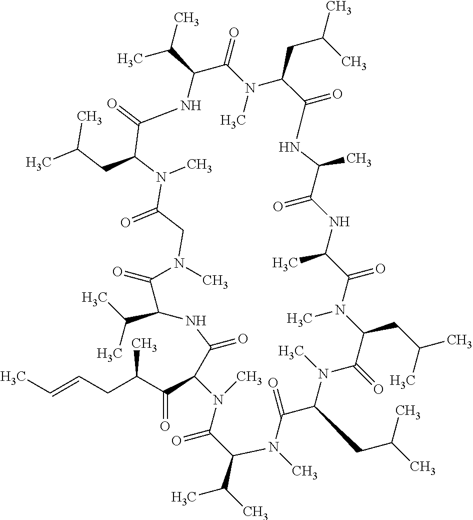 [SMILES: C/C=C/C[C@@H](C)C(=O)[C@H]1C(=O)N[C@@H](C(C)C)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C] | EP 296122 |
| A47 | Vatalanib succinate | 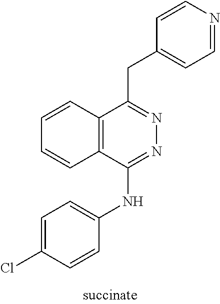 [SMILES: Clc1ccc(Nc2nnc(Cc3ccncc3)c3ccccc23)cc1] | WO 98/35958 |
| A48 | | IDH inhibitor | WO 2014/141104 |
| A49 | | 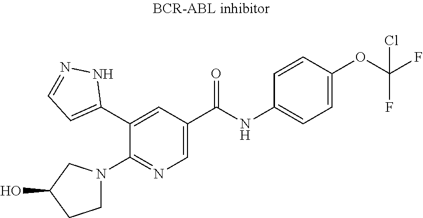 [SMILES: O=C(Nc1ccc(OC(F)(F)Cl)cc1)c1cnc(N2CC[C@@H](O)C2)c(-c2ccn[nH]2)c1] | WO 2013/171639 WO 2013/171640 WO 2013/171641 WO 2013/171642 |
| A50 | | cRAF inhibitor | WO 2014/151616 |
| A51 | | ERK 1/2 ATP | WO 2015/066188 |
| | | competitive | |
| | | inhibitor | |

In some embodiments, the additional therapeutic agent is chosen from one or more of: 1) a protein kinase C (PKC) inhibitor; 2) a heat shock protein 90 (HSP90) inhibitor; 3) an inhibitor of a phosphoinositide 3-kinase (PI3K) and/or target of rapamycin (mTOR); 4) an inhibitor of cytochrome P450 (e.g., a CYP17 inhibitor or a 17alpha-Hydroxylase/C17-20 Lyase inhibitor); 5) an iron chelating agent; 6) an aromatase inhibitor; 7) an inhibitor of p53, e.g., an inhibitor of a p53/Mdm2 interaction; 8) an apoptosis inducer; 9) an angiogenesis inhibitor; 10) an aldosterone synthase inhibitor; 11) a smoothened (SMO) receptor inhibitor; 12) a prolactin receptor (PRLR) inhibitor; 13) a Wnt signaling inhibitor; 14) a CDK4/6 inhibitor; 15) a fibroblast growth factor receptor 2 (FGFR2)/fibroblast growth factor receptor 4 (FGFR4) inhibitor; 16) an inhibitor of macrophage colony-stimulating factor (M-CSF); 17) an inhibitor of one or more of c-KIT, histamine release, Flt3 (e.g., FLK2/STK1) or PKC; 18) an inhibitor of one or more of VEGFR-2 (e.g., FLK-1/KDR), PDGFRbeta, c-KIT or Raf kinase C; 19) a somatostatin agonist and/or a growth hormone release inhibitor; 20) an anaplastic lymphoma kinase (ALK) inhibitor; 21) an insulin-like growth factor 1 receptor (IGF-1R) inhibitor; 22) a P-Glycoprotein 1 inhibitor; 23) a vascular endothelial growth factor receptor (VEGFR) inhibitor; 24) a BCR-ABL kinase inhibitor; 25) an FGFR inhibitor; 26) an inhibitor of CYP11B2; 27) a HDM2 inhibitor, e.g., an inhibitor of the HDM2-p53 interaction; 28) an inhibitor of a tyrosine kinase; 29) an inhibitor of c-MET; 30) an inhibitor of JAK; 31) an inhibitor of DAC; 32) an inhibitor of 11(3-hydroxylase; 33) an inhibitor of IAP; 34) an inhibitor of PIM kinase; 35) an inhibitor of Porcupine; 36) an inhibitor of BRAF, e.g., BRAF V600E or wild-type BRAF; 37) an inhibitor of HER3; 38) an inhibitor of MEK; or 39) an inhibitor of a lipid kinase, e.g., as described herein and in Table 18.

Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-ß inhibitor)), a RAF-1 inhibitor, a KIT inhibitor and a RET inhibitor.

In one embodiment, the combination, e.g., a combination comprising an anti-CD73 antibody molecule as described herein, includes or is used in combination with a PKC inhibitor, Sotrastaurin (Compound A1), or a compound disclosed in PCT Publication No. WO 2005/039549, to treat a disorder, e.g., a disorder described herein. In one embodiment, the PKC inhibitor is Sotrastaurin (Compound Al) or a compound disclosed in PCT Publication No. WO 2005/039549. In one embodiment, an anti-CD73 antibody molecule is used in combination with Sotrastaurin (Compound A1), or a compound as described in PCT Publication No. WO 2005/039549, to treat a disorder such as a cancer, a melanoma, a non-Hodgkin lymphoma, an inflammatory bowel disease, transplant rejection, an ophthalmic disorder, or psoriasis.

In one embodiment, the combination, e.g., a combination comprising an anti-CD73 antibody molecule as described herein, includes or is used in combination with a BCR-ABL inhibitor, TASIGNA (Compound A2), or a compound disclosed in PCT Publication No. WO 2004/005281, to treat a disorder, e.g., a disorder described herein. In one embodiment, the BCR-ABL inhibitor is TASIGNA, or a compound disclosed in PCT Publication No. WO 2004/005281. In one embodiment, an anti-CD73 antibody molecule is used in combination with TASIGNA (Compound A2), or a compound as described in PCT Publication No. WO 2004/005281, to treat a disorder such as a lymphocytic leukemia, Parkinson's Disease, a neurologic cancer, a melanoma, a digestive/gastrointestinal cancer, a colorectal cancer, a myeloid leukemia, a head and neck cancer, or pulmonary hypertension.

In another embodiment, the combination, e.g., a combination comprising an anti-CD73 antibody molecule as described herein, includes or is used in combination with an HSP90 inhibitor, to treat a disorder, e.g., a disorder described herein, e.g., a cancer, a multiple myeloma, a non-small cell lung cancer, a lymphoma, a gastric cancer, a breast cancer, a digestive/gastrointestinal cancer, a pancreatic cancer, a colorectal cancer, a solid tumor, or a hematopoiesis disorder.

In another embodiment, the combination, e.g., a combination comprising an anti-CD73 antibody molecule as described herein, is used in combination with an inhibitor of PI3K and/or mTOR, 8-(6-Methoxy-pyridin-3-yl)-3-methyl-1-(4-piperazin-1-yl-3-trifluoromethyl-phenyl)-1,3-dihydro-imidazo[4,5-c]quinolin-2-one (Compound A41), to treat a disorder, e.g., a disorder described herein. In one embodiment, the PI3K and/or mTOR inhibitor is 8-(6-Methoxy-pyridin-3-yl)-3-methyl-1-(4-piperazin-1-yl-3-trifluoromethyl-phenyl)-1,3-dihydro-imidazo[4,5-c]quinolin-2-one (Compound A41). In one embodiment, an anti-CD73 antibody molecule is used in combination with 8-(6-Methoxy-pyridin-3-yl)-3-methyl-1-(4-piperazin-1-yl-3-trifluoromethyl-phenyl)-1,3-dihydro-imidazo[4,5-c] quinolin-2-one (Compound A41), to treat a disorder such as a cancer, a prostate cancer, a leukemia (e.g., lymphocytic leukemia), a breast cancer, a brain cancer, a bladder cancer, a pancreatic cancer, a renal cancer, a solid tumor, or a liver cancer.

In another embodiment, the combination, e.g., a combination comprising an anti-CD73 antibody molecule as described herein, includes or is used in combination with an FGFR inhibitor, 3-(2,6-dichloro-3,5-dimethoxyphenyl)-1-(6-((4-(4-ethylpiperazin-1-yl)phenyl)amino)pyrimidin-4-yl)-1-methylurea (Compound A5) or a compound disclosed in U.S. Pat. No. 8,552,002, to treat a disorder, e.g., a disorder described herein. In one embodiment, the FGFR inhibitor is 3-(2,6-dichloro-3,5-dimethoxyphenyl)-1-(6-((4-(4-ethylpiperazin-1-yl)phenyl)amino)pyrimidin-4-yl)-1-methylurea (Compound A5) or a compound disclosed in U.S. Pat. No. 8,552,002. In one embodiment, an anti-CD73 antibody molecule is used in combination with Compound A5, or a compound as described in U.S. Pat. No. 8,552,002, to treat a disorder such as a digestive/gastrointestinal cancer, a hematological cancer, or a solid tumor. Compound A5 has the following structure:

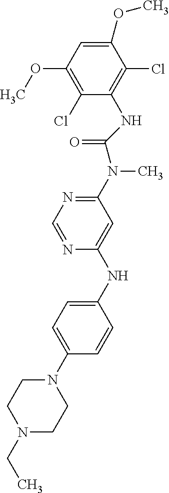 [SMILES: CCN1CCN(c2ccc(Nc3cc(N(C)C(=O)Nc4c(Cl)c(OC)cc(OC)c4Cl)ncn3)cc2)CC1]

In another embodiment, the combination, e.g., a combination comprising an anti-CD73 antibody molecule as described herein, includes or is used in combination with a PI3K inhibitor, Buparlisib (Compound A6), or a compound disclosed in PCT Publication No. WO 2007/084786, to treat a disorder, e.g., a disorder described herein. In one embodiment, the PI3K inhibitor is Buparlisib (Compound A6) or a compound disclosed in PCT Publication No. WO 2007/084786. In one embodiment, an anti-CD73 antibody molecule is used in combination with Buparlisib (Compound A6), or a compound disclosed in PCT Publication No. WO 2007/084786, to treat a disorder such as, a prostate cancer, a non-small cell lung cancer, an endocrine cancer, a leukemia, an ovarian cancer, a melanoma, a bladder cancer, a breast cancer, a female reproductive system cancer, a digestive/gastrointestinal cancer, a colorectal cancer, a glioblastoma multiforme, a solid tumor, a non-Hodgkin lymphoma, a hematopoiesis disorder, or a head and neck cancer. Compound A6 has the following structure:

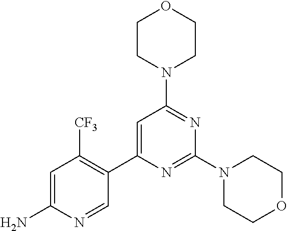 [SMILES: Nc1cc(C(F)(F)F)c(-c2cc(N3CCOCC3)nc(N3CCOCC3)n2)cn1]

In another embodiment, the combination, e.g., a combination comprising an anti-CD73 antibody molecule as described herein, includes or is used in combination with an FGFR inhibitor, 8-(2,6-difluoro-3,5-dimethoxyphenyl)-N-(4-((dimethylamino)methyl)-1H-imidazol-2-yl)quinoxaline-5-carboxamide (Compound A7) or a compound disclosed in PCT Publication No. WO 2009/141386 to treat a disorder, e.g., a disorder described herein. In one embodiment, the FGFR inhibitor is 8-(2,6-difluoro-3,5-dimethoxyphenyl)-N-(4-((dimethylamino)methyl)-1H-imidazol-2-yl)quinoxaline-5-carboxamide(Compound A7) or a compound disclosed in a PCT Publication No. WO 2009/141386. In one embodiment, the FGFR inhibitor is 8-(2,6-difluoro-3,5-dimethoxyphenyl)-N-(4-((dimethylamino)methyl)-1H-imidazol-2-yl)quinoxaline-5-carboxamide(Compound A7). In one embodiment, an anti-CD73 antibody molecule is used in combination with 8-(2,6-difluoro-3,5-dimethoxyphenyl)-N-(4-((dimethylamino)methyl)-1H-imidazol-2-yl)quinoxaline-5-carboxamide(Compound A7), or a compound disclosed in PCT Publication No. WO 2009/141386, to treat a disorder such as a cancer characterized by angiogenesis.

In another embodiment the combination, e.g., a combination comprising an anti-CD73 antibody molecule as described herein, includes or is used in combination with a PI3K inhibitor, (S)—N1-(4-methyl-5-(2-(1,1,1-trifluoro-2-methylpropan-2-yl)pyridin-4-yl)thiazol-2-yl)pyrrolidine-1,2-dicarboxamide (Compound A8) or a compound disclosed PCT Publication No. WO 2010/029082 to treat a disorder, e.g., a disorder described herein. In one embodiment, the PI3K inhibitor is (S)—N1-(4-methyl-5-(2-(1,1,1-trifluoro-2-methylpropan-2-yl)pyridin-4-yl)thiazol-2-yl)pyrrolidine-1,2-dicarboxamide (Compound A8) or a compound disclosed PCT Publication No. WO 2010/029082. In one embodiment, an anti-CD73 antibody molecule is used in combination with (S)—N1-(4-methyl-5-(2-(1,1,1-trifluoro-2-methylpropan-2-yl)pyridin-4-yl)thiazol-2-yl)pyrrolidine-1,2-dicarboxamide (Compound A8), or a compound disclosed PCT Publication No. WO 2010/029082, to treat a disorder such as a gastric cancer, a breast cancer, a pancreatic cancer, a digestive/gastrointestinal cancer, a solid tumor, and a head and neck cancer.

In another embodiment, the combination, e.g., a combination comprising an anti-CD73 antibody molecule as described herein, includes or is used in combination with an inhibitor of cytochrome P450 (e.g., a CYP17 inhibitor) or a compound disclosed in PCT Publication No. WO 2010/149755, to treat a disorder, e.g., a disorder described herein. In one embodiment, the cytochrome P450 inhibitor (e.g., the CYP17 inhibitor) is CFG920 or a compound disclosed in PCT Publication No. WO 2010/149755; U.S. Pat. No. 8,263,635 B2; or EP 2445903 B1. In one embodiment, an anti-CD73 antibody molecule is used in combination with a compound disclosed in PCT Publication No. WO 2010/149755, to treat prostate cancer.

In another embodiment, the combination, e.g., a combination comprising an anti-CD73 antibody molecule as described herein, includes or is used in combination with an HDM2 inhibitor, (S)-1-(4-chlorophenyl)-7-isopropoxy-6-methoxy-2-(4-(methyl(((1r,4S)-4-(4-methyl-3-oxopiperazin-1-yl)cyclohexyl)methyl)amino)phenyl)-1,2-dihydroisoquinolin-3(4H)-one(Compound A10) or a compound disclosed in PCT Publication No. WO 2011/076786 to treat a disorder, e.g., a disorder described herein). In one embodiment, the HDM2 inhibitor is (S)-1-(4-chlorophenyl)-7-isopropoxy-6-methoxy-2-(4-(methyl(((1r,4S)-4-(4-methyl-3-oxopiperazin-1-yl)cyclohexyl)methyl)amino)phenyl)-1,2-dihydroisoquinolin-3(4H)-one (Compound A10) or a compound disclosed in PCT Publication No. WO 2011/076786. In one embodiment, an anti-CD73 antibody molecule is used in combination with (S)-1-(4-chlorophenyl)-7-isopropoxy-6-methoxy-2-(4-(methyl(((1r,4S)-4-(4-methyl-3-oxopiperazin-1-yl)cyclohexyl)methyl)amino)phenyl)-1,2-dihydroisoquinolin-3(4H)-one (Compound A10), or a compound disclosed in PCTPublication No. WO 2011/076786, to treat a disorder such as a solid tumor.

In another embodiment, the combination, e.g., a combination comprising an anti-CD73 antibody molecule as described herein, includes or is used in combination with an iron chelating agent, Deferasirox (also known as EXJADE; Compound A11), or a compound disclosed in PCT Publication No. WO 1997/049395 to treat a disorder, e.g., a disorder described herein. In one embodiment, the iron chelating agent is Deferasirox or a compound disclosed in PCT Publication No. WO 1997/049395. In one embodiment, the iron chelating agent is Deferasirox (Compound A11). In one embodiment, an anti-CD73 antibody molecule is used in combination with Deferasirox (Compound A11), or a compound disclosed in PCT Publication No. WO 1997/049395, to treat iron overload, hemochromatosis, or myelodysplasia.

In another embodiment, the combination, e.g., a combination comprising an anti-CD73 antibody molecule as described herein, includes or is used in combination with an aromatase inhibitor, Letrozole (also known as FEMARA; Compound A12), or a compound disclosed in U.S. Pat. No. 4,978,672 to treat a disorder, e.g., a disorder described herein. In one embodiment, the aromatase inhibitor is Letrozole (Compound A12) or a compound disclosed in U.S. Pat. No. 4,978,672. In one embodiment, an anti-CD73 antibody molecule is used in combination with Letrozole (Compound A12), or a compound disclosed in U.S. Pat. No. 4,978,672, to treat a disorder such as a cancer, a leiomyosarcoma, an endometrium cancer, a breast cancer, a female reproductive system cancer, or a hormone deficiency.

In another embodiment, the combination, e.g., a combination comprising an anti-CD73 antibody molecule as described herein, includes or is used in combination with a PI3K inhibitor, e.g., a pan-PI3K inhibitor, (45,5R)-3-(2′-amino-2-morpholino-4′-(trifluoromethyl)-[4,5′-bipyrimidin]-6-yl)-4-(hydroxymethyl)-5-methyloxazolidin-2-one (Compound A13) or a compound disclosed in PCT Publication No. WO2013/124826 to treat a disorder, e.g., a disorder described herein. In one embodiment, the PI3K inhibitor is (4S,5R)-3-(2′-amino-2-morpholino-4′-(trifluoromethyl)-[4,5′-bipyrimidin]-6-yl)-4-(hydroxymethyl)-5-methyloxazolidin-2-one (Compound A13) or a compound disclosed in PCT Publication No. WO2013/124826. In one embodiment, an anti-CD73 antibody molecule is used in combination with (4S,5R)-3-(2′-amino-2-morpholino-4′-(trifluoromethyl)-[4,5′-bipyrimidin]-6-yl)-4-(hydroxymethyl)-5-methyloxazolidin-2-one (Compound A13), or a compound disclosed in PCT Publication No. WO2013/124826, to treat a disorder such as a cancer or an advanced solid tumor.

In another embodiment, the combination, e.g., a combination comprising an anti-CD73 antibody molecule as described herein, includes or is used in combination with an inhibitor of p53 and/or a p53/Mdm2 interaction, (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one (Compound A14), or a compound disclosed in PCT Publication No. WO2013/111105 to treat a disorder, e.g., a disorder described herein. In one embodiment, the p53 and/or a p53/Mdm2 interaction inhibitor is (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one (Compound A14) or a compound disclosed in PCT Publication No. WO2013/111105. In one embodiment, an anti-CD73 antibody molecule is used in combination with (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one (Compound A14), or a compound disclosed in PCT Publication No. WO2013/111105, to treat a disorder such as a cancer or a soft tissue sarcoma.

In another embodiment, the combination, e.g., a combination comprising an anti-CD73 antibody molecule as described herein, includes or is used in combination with a CSF-1R tyrosine kinase inhibitor, 4-((2-(((1R,2R)-2-hydroxycyclohexyl)amino)benzo[d]thiazol-6-yl)oxy)-N-methylpicolinamide (Compound A15), or a compound disclosed in PCT Publication No. WO 2005/073224 to treat a disorder, e.g., a disorder described herein. In one embodiment, the CSF-1R tyrosine kinase inhibitor is 4-((2-(((1R,2R)-2-hydroxycyclohexyl)amino)benzo[d]thiazol-6-yl)oxy)-N-methylpicolinamide (Compound A15) or a compound disclosed in PCT Publication No. WO 2005/073224. In one embodiment, an anti-CD73 antibody molecule is used in combination with 4-((2-(((1R,2R)-2-hydroxycyclohexyl)amino)benzo[d]thiazol-6-yl)oxy)-N-methylpicolinamide (Compound A15) or a compound disclosed in PCT Publication No. WO 2005/073224, to treat a disorder such as cancer.

In certain embodiments, the CSF-1R tyrosine kinase inhibitor, 4-((2-(((1R,2R)-2-hydroxycyclohexyl)amino)benzo[d]thiazol-6-yl)oxy)-N-methylpicolinamide (Compound A15), or a compound disclosed in PCT Publication No. WO 2005/073224, is administered in combination with the CD73 inhibitor (e.g., the anti-CD73 antibody molecule) to treat a cancer, e.g., a solid tumor (e.g., an advanced solid tumor). Exemplary cancers that can be treated by the combination include, but are not limited to, a brain cancer (e.g., glioblastoma multiforme (GBM), e.g., recurrent glioblastoma), a breast cancer (e.g., a triple-negative breast cancer (e.g., NTBC)), or a pancreatic cancer (e.g., advanced pancreatic cancer). The common features of these cancers include, e.g., a tumor biology characterized by high levels of TAMs in the tumor microenvironment that may contribute to immune evasion and immune suppression. In some embodiments, blockade of CSF-1R in conjunction with an anti-CD73 therapy can, e.g., promote re-programming of TAMs and/or remove immune suppression of tumor infiltrating lymphocytes (TIL).

In another embodiment, the combination, e.g., a combination comprising an anti-CD73 antibody molecule as described herein, includes or is used in combination with an apoptosis inducer and/or an angiogenesis inhibitor, such as Imatinib mesylate (also known as GLEEVEC®; Compound A16) or a compound disclosed in PCT Publication No. WO1999/003854 to treat a disorder, e.g., a disorder described. In one embodiment, the apoptosis inducer and/or an angiogenesis inhibitor is Imatinib mesylate (Compound A16) or a compound disclosed in PCT Publication No. WO1999/003854. In one embodiment, an anti-CD73 antibody molecule is used in combination with Imatinib mesylate (Compound A16), or a compound disclosed in PCT Publication No. WO1999/003854, to treat a disorder such as a cancer, a multiple myeloma, a prostate cancer, a non-small cell lung cancer, a lymphoma, a gastric cancer, a melanoma, a breast cancer, a pancreatic cancer, a digestive/gastrointestinal cancer, a colorectal cancer, a glioblastoma multiforme, a liver cancer, a head and neck cancer, asthma, multiple sclerosis, allergy, Alzheimer's dementia, amyotrophic lateral sclerosis, or rheumatoid arthritis.

In another embodiment, the combination, e.g., a combination comprising an anti-CD73 antibody molecule as described herein, includes or is used in combination with a JAK inhibitor, 2-fluoro-N-methyl-4-(7-(quinolin-6-ylmethyl)imidazo[1,2-b][1,2,4]triazin-2-yl)benzamide (Compound A17), or a dihydrochloric salt thereof, or a compound disclosed in PCT Publication No. WO 2007/070514, to treat a disorder, e.g., a disorder described herein. In one embodiment, the JAK inhibitor is 2-fluoro-N-methyl-4-(7-(quinolin-6-ylmethyl)imidazo[1,2-b][1,2,4]triazin-2-yl)benzamide (Compound A17), or a dihydrochloric salt thereof, or a compound disclosed in PCT Publication No. WO 2007/070514. In one embodiment, an anti-CD73 antibody molecule is used in combination with 2-fluoro-N-methyl-4-(7-(quinolin-6-ylmethyl)imidazo[1,2-b][1,2,4]triazin-2-yl)benzamide (Compound A17), or a dihydrochloric salt thereof, or a compound disclosed in PCT Publication No. WO 2007/070514, to treat a disorder such as colorectal cancer, myeloid leukemia, hematological cancer, autoimmune disease, non-Hodgkin lymphoma, or thrombocythemia.

In another embodiment, the combination, e.g., a combination comprising an anti-CD73 antibody molecule as described herein, includes or is used in combination with a JAK inhibitor, Ruxolitinib Phosphate (also known as JAKAFI; Compound A18) or a compound disclosed in PCT Publication No. WO 2007/070514 to treat a disorder, e.g., a disorder described herein. In one embodiment, the JAK inhibitor is Ruxolitinib Phosphate (Compound A18) or a compound disclosed in PCT Publication No. WO 2007/070514. In one embodiment, an anti-CD73 antibody molecule is used in combination with Ruxolitinib Phosphate (Compound A18), or a compound disclosed in PCT Publication No. WO 2007/070514, to treat a disorder such as a prostate cancer, a lymphocytic leukemia, a multiple myeloma, a lymphoma, a lung cancer, a leukemia, cachexia, a breast cancer, a pancreatic cancer, rheumatoid arthritis, psoriasis, a colorectal cancer, a myeloid leukemia, a hematological cancer, an autoimmune disease, a non-Hodgkin lymphoma, or thrombocythemia.

In another embodiment, the combination, e.g., a combination comprising an anti-CD73 antibody molecule as described herein, includes or is used in combination with a deacetylase (DAC) inhibitor, Panobinostat (Compound A19), or a compound disclosed in PCT Publication No. WO 2014/072493 to treat a disorder, e.g., a disorder described herein. In one embodiment, the DAC inhibitor is Panobinostat (Compound A19) or a compound disclosed in PCT Publication No. WO 2014/072493. In one embodiment, an anti-CD73 antibody molecule is used in combination with Panobinostat (Compound A19), a compound disclosed in PCT Publication No. WO 2014/072493, to treat a disorder such as a colorectal cancer, a small cell lung cancer, a respiratory/thoracic cancer, a prostate cancer, a multiple myeloma, myelodysplastic syndrome, a bone cancer, a non-small cell lung cancer, an endocrine cancer, a lymphoma, a neurologic cancer, a leukemia, HIV/AIDS, an immune disorder, transplant rejection, a gastric cancer, a melanoma, a breast cancer (e.g., a triple negative breast cancer (TNBC)), a pancreatic cancer, a colorectal cancer, a glioblastoma multiforme, a myeloid leukemia, a hematological cancer, a renal cancer, a non-Hodgkin lymphoma, a head and neck cancer, a hematopoiesis disorders, or a liver cancer. In some embodiments, the cancer is chosen from a colorectal cancer (e.g., a microsatellite stable colorectal cancer (MSS CRC), a lung cancer (e.g., a non-small cell lung cancer), or a breast cancer (e.g., a triple negative lung cancer (TNBC)).

In some embodiments, the combination described herein includes a deacetylase (DAC) inhibitor, Panobinostat (Compound A19), or a compound disclosed in PCT Publication No. WO 2014/072493, and an inhibitor of an immune checkpoint molecule, e.g., an inhibitor of CD73 (e.g., an anti-CD73 antibody molecule).

In one embodiment, the DAC inhibitor, Panobinostat (Compound A19), or a compound disclosed in PCT Publication No. WO 2014/072493, is administered in combination with the CD73 inhibitor (e.g., the anti-CD73 antibody molecule) to treat a colorectal cancer (e.g., an MSS CRC), a lung cancer (e.g., a non-small cell lung cancer (NSCLC), or a breast cancer (e.g., a triple negative breast cancer (TNBC)).

In another embodiment, the combination, e.g., a combination comprising an anti-CD73 antibody molecule as described herein, includes or is used in combination with an inhibitor of one or more of cytochrome P450 (e.g., 11B2), aldosterone or angiogenesis, Osilodrostat (Compound A20), or a compound disclosed in PCT Publication No. WO2007/024945 to treat a disorder, e.g., a disorder described herein. In one embodiment, the inhibitor of one or more of cytochrome P450 (e.g., 11B2), aldosterone or angiogenesis is Osilodrostat (Compound A20) or a compound disclosed in PCT Publication No. WO2007/024945. In one embodiment, an anti-CD73 antibody molecule is used in combination with Osilodrostat (Compound A20), or a compound disclosed in PCT Publication No. WO2007/024945, to treat a disorder such as Cushing's syndrome, hypertension, or heart failure therapy.

In another embodiment, the combination, e.g., a combination comprising an anti-CD73 antibody molecule as described herein, includes or is used in combination with a IAP inhibitor, (S)—N—((S)-1-cyclohexyl-2-((S)-2-(4-(4-fluorobenzoyl)thiazol-2-yl)pyrrolidin-1-yl)-2-oxoethyl)-2-(methylamino)propanamide (Compound A21) or a compound disclosed in U.S. Pat. No. 8,552,003 to treat a disorder, e.g., a disorder described herein. In one embodiment, the IAP inhibitor is (S)—N—((S)-1-cyclohexyl-2-((S)-2-(4-(4-fluorobenzoyl)thiazol-2-yl)pyrrolidin-1-yl)-2-oxoethyl)-2-(methylamino)propanamide (Compound A21) or a compound disclosed in U.S. Pat. No. 8,552,003. In one embodiment, an anti-CD73 antibody molecule is used in combination with (S)—N—((S)-1-cyclohexyl-2-((S)-2-(4-(4-fluorobenzoyl)thiazol-2-yl)pyrrolidin-1-yl)-2-oxoethyl)-2-(methylamino)propanamide (Compound A21), or a compound disclosed in U.S. Pat. No. 8,552,003, to treat a disorder such as a multiple myeloma, a colorectal cancer (CLC), a lung cancer (e.g., non-small cell lung cancer (NSCLC), a breast cancer (e.g., a triple-negative breast cancer (TNBC)), an ovarian cancer, a pancreatic cancer, or a hematopoiesis disorder. In some embodiments, the cancer is chosen from a colorectal cancer (e.g., a microsatellite stable colorectal cancer (MSS CRC), a lung cancer (e.g., a non-small cell lung cancer), or a breast cancer (e.g., a triple negative lung cancer (TNBC)).

In another embodiment, the combination, e.g., a combination comprising an anti-CD73 antibody molecule as described herein, includes or is used in combination a Smoothened (SMO) inhibitor, (R)-2-(5-(4-(6-benzyl-4,5-dimethylpyridazin-3-yl)-2-methylpiperazin-1-yl)pyrazin-2-yl)propan-2-ol (Compound A25), or a compound disclosed in PCT Publication No. WO 2010/007120 to treat a disorder, e.g., a disorder described herein. In one embodiment, the SMO inhibitor is (R)-2-(5-(4-(6-benzyl-4,5-dimethylpyridazin-3-yl)-2-methylpiperazin-1-yl)pyrazin-2-yl)propan-2-ol (Compound A25), or a compound disclosed in PCT Publication No. WO 2010/007120. In one embodiment, an anti-CD73 antibody molecule is used in combination with (R)-2-(5-(4-(6-benzyl-4,5-dimethylpyridazin-3-yl)-2-methylpiperazin-1-yl)pyrazin-2-yl)propan-2-ol (Compound A25), or a compound disclosed in PCT Publication No. WO 2010/007120 to treat a disorder such as a cancer, a medulloblastoma, a small cell lung cancer, a prostate cancer, a basal cell carcinoma, a pancreatic cancer, or an inflammation.

In another embodiment, the combination, e.g., a combination comprising an anti-CD73 antibody molecule as described herein, includes or is used in combination with an Alk inhibitor, cen dal (also known as ZYKADIA: Compound A23) to treat a disorder, e.g., a disorder described herein. In one embodiment, the Alk inhibitor is ceritinib (Compound A23). In one embodiment, an anti-CD73 antibody molecule is used in combination with ceritinib (Compound A23), to treat a disorder such as non-small cell lung cancer or solid tumors.

In another embodiment, the combination, e.g., a combination comprising an anti-CD73 antibody molecule as described herein, includes or is used in combination with a JAK and/or CDK4/6 inhibitor, 7-cyclopentyl-N,N-dimethyl-2-((5-(piperazin-1-yl)pyridin-2-yl)amino)-7H-pyrrolo[2,3-d]pyrimidine-6-carboxamide (Compound A24), or a compound disclosed in U.S. Pat. No. 8,415,355 or 8,685,980 to treat a disorder, e.g., a disorder described herein. In one embodiment, the JAK and/or CDK4/6 inhibitor is 7-cyclopentyl-N,N-dimethyl-2-((5-(piperazin-1-yl)pyridin-2-yl)amino)-7H-pyrrolo[2,3-d]pyrimidine-6-carboxamide (Compound A24) or a compound disclosed in U.S. Pat. No. 8,415,355 or 8,685,980. In one embodiment, an anti-CD73 antibody molecule is used in combination with 7-cyclopentyl-N,N-dimethyl-2-((5-(piperazin-1-yl)pyridin-2-yl)amino)-7H-pyrrolo[2,3-d]pyrimidine-6-carboxamide (Compound A24), or a compound disclosed in U.S. Pat. No. 8,415,355 or 8,685,980, to treat a disorder such as a lymphoma, a neurologic cancer, a melanoma, a breast cancer, or a solid tumor.

In another embodiment, the combination, e.g., a combination comprising an anti-CD73 antibody molecule as described herein, includes or is used in combination a prolactin receptor (PRLR) inhibitor, a human monoclonal antibody molecule (Compound A26) as disclosed in U.S. Pat. No. 7,867,493), to treat a disorder, e.g., a disorder described herein. In one embodiment, the PRLR inhibitor is a human monoclonal antibody (Compound A26) disclosed in U.S. Pat. No. 7,867,493. In one embodiment, an anti-CD73 antibody molecule is used in combination with human monoclonal antibody molecule (Compound A26) described in U.S. Pat. No. 7,867,493 to treat a disorder such as, a cancer, a prostate cancer, or a breast cancer.

In another embodiment, the combination, e.g., a combination comprising an anti-CD73 antibody molecule as described herein, includes or is used in combination with a PIM Kinase inhibitor, N-(4-((1R,3S,5S)-3-amino-5-methylcyclohexyl)pyridin-3-yl)-6-(2,6-difluorophenyl)-5-fluoropicolinamide (Compound A27) or a compound disclosed in PCT Publication No. WO 2010/026124 to treat a disorder, e.g., a disorder described herein. In one embodiment, the PIM Kinase inhibitor is N-(4-((1R,35,55)-3-amino-5-methylcyclohexyl)pyridin-3-yl)-6-(2,6-difluorophenyl)-5-fluoropicolinamide (Compound A27) or a compound disclosed in PCT Publication No. WO 2010/026124. In one embodiment, an anti-CD73 antibody molecule is used in combination with N-(4-((1R,3S,5S)-3-amino-5-methylcyclohexyl)pyridin-3-yl)-6-(2,6-difluorophenyl)-5-fluoropicolinamide (Compound A27), or a compound disclosed in PCT Publication No. WO 2010/026124, to treat a disorder such as a multiple myeloma, myelodysplastic syndrome, a myeloid leukemia, or a non-Hodgkin lymphoma.

In another embodiment, the combination, e.g., a combination comprising an anti-CD73 antibody molecule as described herein, includes or is used in combination a Wnt signaling inhibitor, 2-(2′,3-dimethyl-[2,4′-bipyridin]-5-yl)-N-(5-(pyrazin-2-yl)pyridin-2-yl)acetamide (Compound A28) or a compound disclosed in PCT publication No. WO 2010/101849 to treat a disorder, e.g., a disorder described herein. In one embodiment, the Wnt signaling inhibitor is 2-(2′,3-dimethyl-[2,4′-bipyridin]-5-yl)-N-(5-(pyrazin-2-yl)pyridin-2-yl)acetamide (Compound A28) or a compound disclosed in PCT publication No. WO 2010/101849. In one embodiment, the Wnt signaling inhibitor is 2-(2′,3-dimethyl-[2,4′-bipyridin]-5-yl)-N-(5-(pyrazin-2-yl)pyridin-2-yl)acetamide (Compound A28). In one embodiment, an anti-CD73 antibody molecule is used in combination with 2-(2′,3-dimethyl-[2,4′-bipyridin]-5-yl)-N-(5-(pyrazin-2-yl)pyridin-2-yl)acetamide (Compound A28), or a compound disclosed in PCT publication No. WO 2010/101849, to treat a disorder such as a solid tumor (e.g., a head and neck cancer, a squamous cell carcinoma, a breast cancer, a pancreatic cancer, or a colon cancer). In certain embodiments, the cancer is chosen from a skin cancer (e.g., a melanoma), a microsatellite instability-high (MSI-high) solid tumor, a pancreatic cancer, or a breast cancer (e.g., a triple negative breast cancer (TNBC)).

In another embodiment, the combination, e.g., a combination comprising an anti-CD73 antibody molecule as described herein, includes or is used in combination with a BRAF inhibitor, to treat a disorder, e.g., a disorder described herein, e.g., a non-small cell lung cancer, a melanoma, or a colorectal cancer.

In another embodiment, the combination, e.g., a combination comprising an anti-CD73 antibody molecule as described herein, includes or is used in combination a CDK4/6 inhibitor, 7-cyclopentyl-N,N-dimethyl-2-((5-((1R,6S)-9-methyl-4-oxo-3,9-diazabicyclo[4.2.1]nonan-3-yl)pyridin-2-yl)amino)-7H-pyrrolo[2,3-d]pyrimidine-6-carboxamide (Compound A30), or a compound disclosed in PCT publication No. WO 2011/101409 to treat a disorder, e.g., a disorder described herein. In one embodiment, the CDK4/6 inhibitor is 7-cyclopentyl-N,N-dimethyl-2-((5-((1R,6S)-9-methyl-4-oxo-3,9-diazabicyclo[4.2.1]nonan-3-yl)pyridin-2-yl)amino)-7H-pyrrolo[2,3-d]pyrimidine-6-carboxamide (Compound A30) or a compound disclosed in PCT publication No. WO 2011/101409. In one embodiment, an anti-CD73 antibody molecule is used in combination with 7-cyclopentyl-N,N-dimethyl-2-((5-((1R,6S)-9-methyl-4-oxo-3,9-diazabicyclo[4.2.1]nonan-3-yl)pyridin-2-yl)amino)-7H-pyrrolo[2,3-d]pyrimidine-6-carboxamide (Compound A30), or a compound disclosed in PCT publication No. WO 2011/101409, to treat a disorder such as a cancer, a mantle cell lymphoma, a liposarcoma, a non-small cell lung cancer, a melanoma, a squamous cell esophageal cancer, or a breast cancer.

In another embodiment, the combination, e.g., a combination comprising an anti-CD73 antibody molecule as described herein, includes or is used in combination with a HER3 inhibitor, Compound A31, or a compound disclosed in PCT Publication No. WO 2012/022814, to treat a disorder, e.g., a disorder described herein. In one embodiment, the HER3 inhibitor is Compound A31 or a compound disclosed in PCT Publication WO 2012/022814. In one embodiment, an anti-CD73 antibody molecule is used in combination with Compound A31, or a compound disclosed in PCT Publication WO 2012/022814, to treat a disorder such as a gastric cancer, an esophageal cancer, a head and neck cancer, a squamous cell carcinoma, a stomach cancer, a breast cancer (e.g., metastatic breast cancer), or a digestive/gastrointestinal cancer.

In another embodiment, the combination, e.g., a combination comprising an anti-CD73 antibody molecule as described herein, includes or is used in combination an FGFR2 and/or FGFR4 inhibitor, Compound A32, or a compound disclosed in a publication PCT Publication No. WO 2014/160160 (e.g., an antibody molecule drug conjugate against an FGFR2 and/or FGFR4, e.g., mAb 12425), to treat a disorder, e.g., a disorder described herein. In one embodiment, the FGFR2 and/or FGFR4 inhibitor is Compound A32 or a compound disclosed in a publication PCT Publication No. WO 2014/160160. In one embodiment, an anti-CD73 antibody molecule is used in combination with Compound A32, or a compound as described in Table 18, to treat a disorder such as a cancer, a gastric cancer, a breast cancer, a rhabdomyosarcoma, a liver cancer, an adrenal cancer, a lung cancer, an esophageal cancer, a colon cancer, or an endometrial cancer.

In some embodiments, Compound A32 is an antibody molecule drug conjugate against an FGFR2 and/or FGFR4, e.g., mAb 12425.

In another embodiment, the combination, e.g., a combination comprising an anti-CD73 antibody molecule as described herein, includes or is used in combination an M-CSF inhibitor, Compound A33, or a compound disclosed in PCT Publication No. WO 2004/045532 (e.g., an antibody molecule or Fab fragment against M-CSF), to treat a disorder, e.g., a disorder described herein. In one embodiment, the M-CSF inhibitor is Compound A33 or a compound disclosed in PCT Publication No. WO 2004/045532. In one embodiment, an anti-CD73 antibody molecule is used in combination with Compound A33, or a compound as described in PCT Publication No. WO 2004/045532, to treat a disorder such as a cancer, a prostate cancer, a breast cancer, or pigmented villonodular synovitis (PVNS).

In another embodiment, the combination, e.g., a combination comprising an anti-CD73 antibody molecule as described herein, includes or is used in combination with a MEK inhibitor, to treat a disorder such as a non-small cell lung cancer, a multisystem genetic disorder, a melanoma, an ovarian cancer, a digestive/gastrointestinal cancer, a rheumatoid arthritis, or a colorectal cancer.

In another embodiment, the combination, e.g., a combination comprising an anti-CD73 antibody molecule as described herein, includes or is used in combination an inhibitor of one or more of c-KIT, histamine release, Flt3 (e.g., FLK2/STK1) or PKC, Midostaurin (Compound A35) or a compound disclosed in PCT Publication No. WO 2003/037347 to treat a disorder, e.g., a disorder described herein. In one embodiment, the inhibitor is Midostaurin (Compound A35) or compound disclosed in PCT Publication No. WO 2003/037347. In one embodiment, the inhibitor of one or more of c-KIT, histamine release, Flt3 (e.g., FLK2/STK1) or PKC is Midostaurin. In one embodiment, an anti-CD73 antibody molecule is used in combination with Midostaurin (Compound A35), or compound disclosed in PCT Publication No. WO 2003/037347, to treat a disorder such as a cancer, a colorectal cancer, a myeloid leukemia, myelodysplastic syndrome, an age-related macular degeneration, a diabetic complication, or a dermatologic disorder.

In another embodiment, the combination, e.g., a combination comprising an anti-CD73 antibody molecule as described herein, includes or is used in combination with a TOR inhibitor (e.g., mTOR inhibitor), Everolimus (also known as AFINITOR; Compound A36) or a Compound disclosed in PCT Publication No. WO 2014/085318 to treat a disorder, e.g., a disorder described herein). In one embodiment, the TOR inhibitor is Everolimus (Compound A36) or a Compound disclosed in PCT Publication No. WO 2014/085318. In one embodiment, an anti-CD73 antibody molecule is used in combination with Everolimus (Compound A36) to treat a disorder such as a colorectal cancer, an interstitial lung disease, a small cell lung cancer, a respiratory/thoracic cancer, a prostate cancer, a multiple myeloma, a sarcoma, an age-related macular degeneration, a bone cancer, tuberous sclerosis, a non-small cell lung cancer, an endocrine cancer, a lymphoma, a neurologic disorders, an astrocytoma, a cervical cancer, a neurologic cancer, a leukemia, an immune disorders, transplant rejection, a gastric cancer, a melanoma, epilepsy, a breast cancer (e.g., a triple-negative breast cancer (TNBC), or a bladder cancer. In some embodiments, the cancer is chosen from a colorectal cancer (e.g., a microsatellite stable colorectal cancer (MSS CRC), a lung cancer (e.g., a non-small cell lung cancer), or a breast cancer (e.g., a triple negative lung cancer (TNBC)).

In some embodiments, the combination described herein includes the mTOR inhibitor, everolimus (Compound A36), or a compound disclosed in PCT Publication No. WO 2014/085318, and an inhibitor of an immune checkpoint molecule, e.g., an inhibitor of CD73 (e.g., an anti-CD73 antibody molecule).

In another embodiment, the combination, e.g., a combination comprising an anti-CD73 antibody molecule as described herein, includes or is used in combination an inhibitor of one or more of VEGFR-2, PDGFRbeta, KIT or Raf kinase C, 1-methyl-5-((2-(5-(trifluoromethyl)-1H-imidazol-2-yl)pyridin-4-yl)oxy)-N-(4-(trifluoromethyl)phenyl)-1H-benzo[d]imidazol-2-amine (Compound A37) or a compound disclosed in PCT Publication No. WO 2007/030377 to treat a disorder, e.g., a disorder described herein. In one embodiment, the inhibitor of one or more of VEGFR-2, PDGFRbeta, KIT or Raf kinase C is 1-methyl-54(2-(5-(trifluoromethyl)-1H-imidazol-2-yl)pyridin-4-yl)oxy)-N-(4-(trifluoromethyl)phenyl)-1H-benzo[d]imidazol-2-amine (Compound A37) or a compound disclosed in PCT Publication No. WO 2007/030377. In one embodiment, an anti-CD73 antibody molecule is used in combination with 1-methyl-5-(2-(5-(trifluoromethyl)-1H-imidazol-2-yl)pyridin-4-yl)oxy)-N-(4-(trifluoromethyl)phenyl)-1H-benzo[d]imidazol-2-amine (Compound A37), or a compound disclosed in PCT Publication No. WO 2007/030377, to treat a disorder such as a cancer, a melanoma, or a solid tumor.

In another embodiment, the combination, e.g., a combination comprising an anti-CD73 antibody molecule as described herein, includes or is used in combination a somatostatin agonist and/or growth hormone release inhibitor, Pasireotide diaspartate (also known as SIGNIFOR; Compound A38) or a compound disclosed in PCT Publication No. WO2002/010192 or U.S. Pat. No. 7,473,761 to treat a disorder, e.g., a disorder described herein. In one embodiment, the somatostatin agonist and/or growth hormone release inhibitor is Pasireotide diaspartate (Compound A38) or a compound disclosed in PCT Publication No. WO2002/010192 or U.S. Pat. No. 7,473,761. In one embodiment, an anti-CD73 antibody molecule is used in combination with Pasireotide diaspartate (Compound A38), or a compound disclosed in PCT Publication No. WO2002/010192 or U.S. Pat. No. 7,473,761, to treat a disorder such as a prostate cancer, an endocrine cancer, a nurologic cancer, a neuroendocrine tumor (NET) (e.g., an atypical pulmonary carcinoid tumor), a skin cancer (e.g., a melanoma or Merkel cell carcinoma), a pancreatic cancer, a liver cancer, Cushing's syndrome, a gastrointestinal disorder, acromegaly, a liver and biliary tract disorder, or liver cirrhosis.

In another embodiment, the combination, e.g., a combination comprising an anti-CD73 antibody molecule as described herein, includes or is used in combination a signal transduction modulator and/or angiogenesis inhibitor, e.g., to treat a disorder such as a cancer, a respiratory/thoracic cancer, a multiple myeloma, a prostate cancer, a non-small cell lung cancer, an endocrine cancer, or a neurological genetic disorder.

In another embodiment, the combination, e.g., a combination comprising an anti-CD73 antibody molecule as described herein, includes or is used in combination with an EGFR inhibitor, (R,E)-N-(7-chloro-1-(1-(4-(dimethylamino)but-2-enoyl)azepan-3-yl)-1H-benzo[d]imidazol-2-yl)-2-methylisonicotinamide (Compound A40) or a compound disclosed in PCT Publication No. WO 2013/184757 to treat a disorder, e.g., a disorder described herein. In one embodiment, the EGFR inhibitor is (R,E)-N-(7-chloro-1-(1-(4-(dimethylamino) but-2-enoyl)azepan-3-yl)-1H-benzo[d]imidazol-2-yl)-2-methylisonicotinamide (Compound A40) or a compound disclosed in PCT Publication No. WO 2013/184757. In one embodiment, an anti-CD73 antibody molecule is used in combination with (R,E)-N-(7-chloro-1-(1-(4-(dimethylamino)but-2-enoyl)azepan-3-yl)-1H-benzo[d]imidazol-2-yl)-2-methylisonicotinamide (Compound A40), or a compound disclosed in PCT Publication No. WO 2013/184757, to treat a disorder such as a cancer, e.g., a solid tumor.

In some embodiments, the EGFR inhibitor, (R,E)-N-(7-chloro-1-(1-(4-(dimethylamino)but-2-enoyl)azepan-3-yl)-1H-benzo[d]imidazol-2-yl)-2-methylisonicotinamide (Compound A40), or a compound disclosed in PCT Publication No. WO 2013/184757, is administered in combination with an inhibitor of CD73 (e.g., an anti-CD73 antibody molecule) to treat a colorectal cancer (CRC), a lung cancer (e.g., a non-small cell lung cancer (NSCLC)), or a breast cancer (e.g., a triple negative breast cancer (TNBC)).

In another embodiment, the combination, e.g., a combination comprising an anti-CD73 antibody molecule as described herein, includes or is used in combination an ALK inhibitor, N⁶-(2-isopropoxy-5-methyl-4-(1-methylpiperidin-4-yl)phenyl)-N⁴-(2-(isopropylsulfonyl)phenyl)-1H-pyrazolo[3,4-d]pyrimidine-4,6-diamine (Compound A42) or a compound disclosed in PCT Publication No. WO 2008/073687 to treat a disorder, e.g., a disorder described herein. In one embodiment, the ALK inhibitor is N⁶-(2-isopropoxy-5-methyl-4-(1-methylpiperidin-4-yl)phenyl)-N⁴-(2-(isopropylsulfonyl)phenyl)-1H-pyrazolo[3,4-d]pyrimidine-4,6-diamine (Compound A42) or a compound disclosed in PCT Publication No. WO 2008/073687. In one embodiment, an anti-CD73 antibody molecule is used in combination with N⁶-(2-isopropoxy-5-methyl-4-(1-methylpiperidin-4-yl)phenyl)-N⁴-(2-(isopropylsulfonyl)phenyl)-1H-pyrazolo[3,4-d]pyrimidine-4,6-diamine (Compound A42), or a compound disclosed in PCT Publication No. WO 2008/073687, to treat a disorder such as a cancer, an anaplastic large-cell lymphoma (ALCL), a non-small cell lung carcinoma (NSCLC), or a neuroblastoma.

In another embodiment, the combination, e.g., a combination comprising an anti-CD73 antibody molecule as described herein, includes or is used in combination an IGF-1R inhibitor, 3-(4-(4-((5-chloro-4-((5-methyl-1H-pyrazol-3-yl)amino)pyrimidin-2-yl)amino)-5-fluoro-2-methylphenyl)piperidin-1-yl)thietane 1,1-dioxide (Compound A43), 5-chloro-N²-(2-fluoro-5-methyl-4-(1-(tetrahydro-2H-pyran-4-yl)piperidin-4-yl)phenyl)-N⁴-(5-methyl-1H-pyrazol-3-yl)pyrimidine-2,4-diamine (Compound A44), or 5-chloro-N2-(4-(1-ethylpiperidin-4-yl)-2-fluoro-5-methylphenyl)-N⁴-(5-methyl-1H-pyrazol-3-yl)pyrimidine-2,4-diamine (Compound A45) or a compound disclosed in PCT Publication No. WO 2010/002655 to treat a disorder, e.g., a disorder described. In one embodiment, the IGF-1R inhibitor is 3-(4-(4-((5-chloro-4-((5-methyl-1H-pyrazol-3-yl)amino)pyrimidin-2-yl)amino)-5-fluoro-2-methylphenyl)piperidin-1-yl)thietane 1,1-dioxide (Compound A43), 5-chloro-N²-(2-fluoro-5-methyl-4-(1-(tetrahydro-2H-pyran-4-yl)piperidin-4-yl)phenyl)-N⁴-(5-methyl-1H-pyrazol-3-yl)pyrimidine-2,4-diamine (Compound A44), 5-chloro-N2-(4-(1-ethylpiperidin-4-yl)-2-fluoro-5-methylphenyl)-N⁴-(5-methyl-1H-pyrazol-3-yl)pyrimidine-2,4-diamine (Compound A45), or a compound disclosed in PCT Publication No. WO 2010/002655. In one embodiment, an anti-CD73 antibody molecule is used in combination with 3-(4-(4-((5-chloro-4-((5-methyl-1H-pyrazol-3-yl)amino)pyrimidin-2-yl)amino)-5-fluoro-2-methylphenyl)piperidin-1-yl)thietane 1,1-dioxide (Compound A43), 5-chloro-N²-(2-fluoro-5-methyl-4-(1-(tetrahydro-2H-pyran-4-yl)piperidin-4-yl)phenyl)-N⁴-(5-methyl-1H-pyrazol-3-yl)pyrimidine-2,4-diamine (Compound A44), 5-chloro-N2-(4-(1-ethylpiperidin-4-yl)-2-fluoro-5-methylphenyl)-N⁴-(5-methyl-1H-pyrazol-3-yl)pyrimidine-2,4-diamine (Compound A45), or a compound disclosed in PCT Publication No. WO 2010/002655, to treat a disorder such as a cancer or a sarcoma.

In another embodiment, the combination, e.g., a combination comprising an anti-CD73 antibody molecule as described herein, includes or is used in combination a P-Glycoprotein 1 inhibitor, Valspodar (also known as AMDRAY; Compound A46) or a compound disclosed in EP 296122 to treat a disorder, e.g., a disorder described herein. In one embodiment, the P-Glycoprotein 1 inhibitor is Valspodar (Compound A46) or a compound disclosed in EP 296122. In one embodiment, an anti-CD73 antibody molecule is used in combination with Valspodar (Compound A46), or a compound disclosed in EP 296122, to treat a disorder such as a cancer or a drug-resistant tumor.

In another embodiment, the combination, e.g., a combination comprising an anti-CD73 antibody molecule as described herein, includes or is used in combination one or more of a VEGFR inhibitor, Vatalanib succinate (Compound A47) or a compound disclosed in EP 296122 to treat a disorder, e.g., a disorder described herein. In one embodiment, the VEGFR inhibitor is Vatalanib succinate (Compound A47) or a compound disclosed in EP 296122. In one embodiment, an anti-CD73 antibody molecule is used in combination with Vatalanib succinate (Compound A47), or a compound disclosed in EP 296122, to treat cancer.

In another embodiment, the combination, e.g., a combination comprising an anti-CD73 antibody molecule as described herein, includes or is used in combination with an IDH inhibitor or a compound disclosed in WO2014/141104 to treat a disorder, e.g., a disorder described herein. In one embodiment, the IDH inhibitor is a compound disclosed in PCT Publication No. WO2014/141104. In one embodiment, an anti-CD73 antibody molecule is used in combination with a compound disclosed in WO2014/141104 to treat a disorder such as a cancer.

In another embodiment, the combination, e.g., a combination comprising an anti-CD73 antibody molecule as described herein, includes or is used in combination with a BCL-ABL inhibitor or a compound disclosed in PCT Publication No. WO2013/171639, WO2013/171640, WO2013/171641, or WO2013/171642 to treat a disorder, e.g., a disorder described herein. In one embodiment, the BCL-ABL inhibitor is a compound disclosed in PCT Publication No. WO2013/171639, WO2013/171640, WO2013/171641, or WO2013/171642. In one embodiment, an anti-CD73 antibody molecule is used in combination with a compound disclosed in PCT Publication No. WO2013/171639, WO2013/171640, WO2013/171641, or WO2013/171642 to treat a disorder such as a cancer.

In another embodiment, the combination, e.g., a combination comprising an anti-CD73 antibody molecule as described herein, includes or is used in combination with a c-RAF inhibitor or a compound disclosed in PCT Publication No. WO2014/151616 to treat a disorder, e.g., a disorder described herein. In one embodiment, the c-RAF inhibitor is Compound A50 or a compound disclosed in PCT Publication No. WO2014/151616. In some embodiments, the c-RAF inhibitor or Compound A50 is a compound of formula (I):

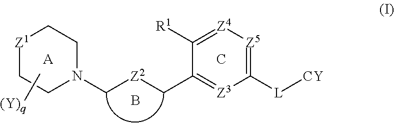

or a pharmaceutically acceptable salt thereof, wherein:

Z¹ is O, S, S(═O) or SO₂;

Z² is N, S or CRa, where Ra is H, halo, C₁₋₄ alkyl or C₁₋₄ haloalkyl;

R¹ is CN, halo, OH, C₁₋₄ alkoxy, or C₁₋₄ alkyl that is optionally substituted with one to three groups selected from halo, C₁₋₄ alkoxy, CN, and hydroxyl;

Ring B is selected from phenyl, pyridine, pyrimidine, pyrazine, pyridazine, pyridone, pyrimidone, pyrazinone, pyridazinone, and thiazole, each of which is optionally substituted with up to two groups selected from halo, OH, ON, C₁₋₄ alkyl, C₂₋₄ alkenyl, —O—(C₁₋₄ alkyl), NH₂, NH—(C₁₋₄ alkyl), —N(C₁₋₄ alkyl)₂, —SO₂R², NHSO₂R², NHC(O)R², NHCO₂R², C₃₋₆ cycloalkyl, 5-6 membered heteroaryl, —O-C3-6 cycloalkyl, —O-(5-6-membered heteroaryl), C₄₋₈ heterocycloalkyi, and —O-(4-8 membered heterocycloalkyi), where each heterocycloalkyi and heteroaryl contains up to three heteroatoms selected from N, O and S as ring members,
where each C₁₋₄ alkyl, C₂₋₄ alkenyl, C₃₋₆ cycloalkyl, 5-6 membered heteroaryl, and 4-8 membered heterocycloalkyl is each optionally substituted with up to three groups selected from oxo, hydroxyl, halo, C₁₋₄ alkyl, C₁₋₄ haloalkyl, C₁₋₄ alkoxy, and —(CH₂)₁₋₂Q where Q is OH, C₁₋₄ alkoxy, —CN, NH₂, —NHR³, —N(R³)₂, —SO₂R³, NHSO₂R³, NHC(O)OR³, or NHC(O)R³; each R² and R³ is independently C14 alkyl; and
Ring B is optionally fused to a 5-6 membered aromatic or nonaromatic ring containing up to two heteroatoms selected from N, O and S, where the 5-6 membered ring can be substituted with halo, C₁₋₄ alkyl, C₁₋₄ haloalkyl, or C₁₋₄ alkoxy, and if the fused ring is non-aromatic the substituent options can further include oxo;

each Y is independently selected from C₁₋₄ alkyl, C₁₋₄ alkoxy, CN, halo, oxo, —(CH₂)ₚOR⁴, —(CH₂)p N(R⁴)₂, —(CH₂)pNHC(O)R⁴, —(CH₂)ₚNHCOO(C₁₋₄ alkyl), and imidazole,

or two Y groups on Ring A are optionally taken together to form a ring fused to or bridging Ring A, where said fused or bridging ring optionally contains a heteroatom selected from N, O and S as a ring member, and is optionally substituted with up to two groups selected from C₁₋₄ alkyl, C₁₋₄ alkoxy, CN, halo, oxo, —(CH₂)ₚOR⁴, —(CH₂)p N(R⁴)₂, —(CH₂)ₚNHC(O)R⁴, and —(CH₂)ₚNHCOO(C₁₋₄ alkyl);

each R⁴ is independently H or C₁₋₄ alkyl;

each p is independently 0, 1, or 2;

q is 0, 1 or 2;

Z³, Z⁴, and Z⁵ are independently selected from CH and N and optionally NO;

L is —C(═O)—NR⁴-[CY] or —NR⁴—C(═O)—[CY], where [CY] indicates which atom of L is attached to CY; and

CY is an aromatic ring selected from phenyl, pyridine, pyrimidine, pyrazine, pyridazine, pyridone, thiazole, isothiazole, oxazole, pyrazole, and isoxazole, wherein the ring is optionally fused to a thiophene, imidazole, oxazolone, or pyrrole ring;

and CY is substituted with up to two groups selected from halo, CN, R⁵, OR^{S}, SO₂R⁵, —S(═NH)(═O)R⁵, OH, NH₂, NHR⁵, and —N(R⁵)₂,
wherein each R⁵ is independently C₁₋₄ alkyl, C₂₋₄. alkenyl, C₂₋₆ heterocyclyl, 5-membered heteroaryl containing up to three heteroatoms selected from N, O and S as ring members, or C₃₋₈ cycloalkyl, and R⁵ is optionally substituted with up to four groups selected from oxo, halo, CN, R⁶, OH, OR⁶, SO₂R⁶, NH₂, NHR⁶, N(R⁶)₂, NHSO₂R⁶, NHCOOR⁶, NHC(═O)R⁶, —CH₂OR⁷, —CH₂N(R⁷)₂, wherein each

R⁶ is independently C₁₋₄ alkyl, and each R⁷ is independently H or C₁₋₄ alkyl;

and two R⁴, R⁵, R⁶, or R⁷ on the same nitrogen atom can be taken together to form a 5-6 membered heterocyclic ring optionally containing an additional N, O or S as a ring member and optionally substituted with up to two groups selected from C₁₋₄ alkyl, oxo, halo, OH, and C₁₋₄ alkoxy.

Methods of administering the antibody molecules are known in the art and are described below. Suitable dosages of the molecules used will depend on the age and weight of the subject and the particular drug used. Dosages and therapeutic regimens of the anti-CD73 antibody molecule can be determined by a skilled artisan.

In certain embodiments, the anti-CD73 antibody molecule is administered by injection (e.g., intravenously) at a dose (e.g., a flat dose) of about 60 mg to 2400 mg, e.g., about 100 mg to 2400 mg, about 100 mg to 2200 mg, about 100 mg to 2000 mg, about 100 mg to 1800 mg, about 100 mg to 1600 mg, about 100 mg to 1400 mg, about 100 mg to 1200 mg, about 100 mg to 1000 mg, about 100 mg to 800 mg, about 100 mg to 600 mg, about 100 mg to 400 mg, about 100 mg to 200 mg, or about 100 mg, about 180 mg, or about 200 mg. The dosing schedule can vary from e.g., once a week to once every 2, 3, or 4 weeks. In some embodiments, the anti-CD73 antibody molecule is administered at a dose of about 100 mg once every two weeks by intravenous infusion. In some embodiments, the anti-CD73 antibody molecule is administered at a dose of at least about 180 mg once every two weeks by intravenous infusion. In some embodiments, the anti-CD73 antibody molecule is administered at a dose of about 200 mg once every two weeks by intravenous infusion.

In some embodiments, an anti-CD73 antibody molecule disclosed herein is administered by injection (e.g., subcutaneously or intravenously) at a dose (e.g., a flat dose) of about 5 mg to 100 mg, about 100 mg to 500 mg, about 500 mg to 1000 mg, about 1000 mg to 1500 mg, about 1500 mg to 2000 mg, about 2000 mg to 2500 mg, about 2500 mg to 3000 mg, about 3000 mg to 3500 mg, or about 3500 mg to 4000 mg, e.g., once every week (QW), once every two weeks (Q2W), or once every four weeks (Q4W). In certain embodiments, the antibody molecule is administered, e.g., intravenously, at a dose of about 6 mg, about 20 mg, about 60 mg, about 200 mg, about 600 mg, about 1200 mg, about 2400 mg, about 3000 mg, or about 3600 mg, e.g., QW, Q2W, or Q4W. In certain embodiments, the antibody molecule is administered, e.g., intravenously, at a dose of about 60 mg Q2W. In certain embodiments, the antibody molecule is administered, e.g., intravenously, at a dose of about 200 mg Q2W. In certain embodiments, the antibody molecule is administered, e.g., intravenously, at a dose of about 600 mg Q2W. In certain embodiments, the antibody molecule is administered, e.g., intravenously, at a dose of about 1200 mg Q2W. In certain embodiments, the antibody molecule is administered, e.g., intravenously, at a dose of about 2400 mg Q2W. In certain embodiments, the antibody molecule is administered, e.g., intravenously, at a dose of about 3000 mg Q2W. In certain embodiments, the antibody molecule is administered, e.g., intravenously, at a dose of about 3600 mg Q2W.

In certain embodiments, the anti-CD73 antibody molecule is administered by injection (e.g., intravenously) at a dose of about 1 to 30 mg/kg, e.g., about 5 to 25 mg/kg, about 10 to 20 mg/kg, about 1 to 5 mg/kg, or about 3 mg/kg. In some embodiments, the anti-CD73 antibody molecule is administered at a dose of about 1 mg/kg, about 3 mg/kg, or 10 mg/kg, about 20 mg/kg, about 30 mg/kg, or about 40 mg/kg. In some embodiments, the anti-CD73 antibody molecule is administered at a dose of about 1-3 mg/kg, or about 3-10 mg/kg. In some embodiments, the anti-CD73 antibody molecule is administered at a dose of about 0.5-2, 2-4, 2-5, 5-15, or 5-20 mg/kg. The dosing schedule can vary from e.g., once a week to once every 2, 3, or 4 weeks. In one embodiment, the anti-CD73 antibody molecule is administered at a dose of about 10 to 20 mg/kg every other week.

The antibody molecules can be used in unconjugated forms or conjugated to a second agent, e.g., a cytotoxic drug, radioisotope, or a protein, e.g., a protein toxin or a viral protein. This method includes: administering the antibody molecule, alone or conjugated to a cytotoxic drug, to a subject requiring such treatment. The antibody molecules can be used to deliver a variety of therapeutic agents, e.g., a cytotoxic moiety, e.g., a therapeutic drug, a radioisotope, molecules of plant, fungal, or bacterial origin, or biological proteins (e.g., protein toxins) or particles (e.g., a recombinant viral particles, e.g., via a viral coat protein), or mixtures thereof.

### Diagnostic Uses

In one aspect, the present invention provides a diagnostic method for detecting the presence of a

CD73 protein in vitro (e.g., in a biological sample, such as a tissue biopsy, e.g., from a cancerous tissue) or in vivo (e.g., in vivo imaging in a subject). The method includes: (i) contacting the sample with an antibody molecule described herein, or administering to the subject, the antibody molecule; (optionally) (ii) contacting a reference sample, e.g., a control sample (e.g., a control biological sample, such as plasma, tissue, biopsy) or a control subject)); and (iii) detecting formation of a complex between the antibody molecule, and the sample or subject, or the control sample or subject, wherein a change, e.g., a statistically significant change, in the formation of the complex in the sample or subject relative to the control sample or subject is indicative of the presence of CD74 in the sample. The antibody molecule can be directly or indirectly labeled with a detectable substance to facilitate detection of the bound or unbound antibody. Suitable detectable substances include various enzymes, prosthetic groups, fluorescent materials, luminescent materials and radioactive materials, as described above and described in more detail below.

The term “sample,” as it refers to samples used for detecting polypeptides includes, but is not limited to, cells, cell lysates, proteins or membrane extracts of cells, body fluids, or tissue samples.

Complex formation between the antibody molecule and CD73 can be detected by measuring or visualizing either the binding molecule bound to the CD73 antigen or unbound binding molecule. Conventional detection assays can be used, e.g., an enzyme-linked immunosorbent assays (ELISA), a radioimmunoassay (RIA) or tissue immunohistochemistry. Alternative to labeling the antibody molecule, the presence of CD73 can be assayed in a sample by a competition immunoassay utilizing standards labeled with a detectable substance and an unlabeled antibody molecule. In this assay, the biological sample, the labeled standards and the antibody molecule are combined and the amount of labeled standard bound to the unlabeled binding molecule is determined. The amount of CD73 in the sample is inversely proportional to the amount of labeled standard bound to the antibody molecule.

### Nucleic Acids

The invention also features nucleic acids comprising nucleotide sequences that encode heavy and light chain variable regions and CDRs or hypervariable loops of the anti-CD73 antibody molecules, as described herein. For example, the invention features a first and second nucleic acid encoding heavy and light chain variable regions, respectively, of an anti-CD73 antibody molecule chosen from one or more of the antibody molecules disclosed herein. The nucleic acid can comprise a nucleotide sequence as set forth in Table 1, or a sequence substantially identical thereto (e.g., a sequence having at least about 85%, 90%, 95%, or 99% sequence identity thereto, or which differs by no more than 3, 6, 15, 30, or 45 nucleotides from the sequences shown in Table 1).

In certain embodiments, the nucleic acid can comprise a nucleotide sequence encoding at least one, two, or three CDRs or hypervariable loops from a heavy chain variable region having an amino acid sequence as set forth in Table 1, or a sequence substantially homologous thereto (e.g., a sequence having at least about 85%, 90%, 95%, or 99% sequence identity thereto, and/or having one or more substitutions, e.g., conserved substitutions). In other embodiments, the nucleic acid can comprise a nucleotide sequence encoding at least one, two, or three CDRs or hypervariable loops from a light chain variable region having an amino acid sequence as set forth in Table 1, or a sequence substantially homologous thereto (e.g., a sequence having at least about 85%, 90%, 95%, or 99% sequence identity thereto, and/or having one or more substitutions, e.g., conserved substitutions). In yet another embodiment, the nucleic acid can comprise a nucleotide sequence encoding at least one, two, three, four, five, or six CDRs or hypervariable loops from heavy and light chain variable regions having an amino acid sequence as set forth in Table 1, or a sequence substantially homologous thereto (e.g., a sequence having at least about 85%, 90%, 95%, or 99% sequence identity thereto, and/or having one or more substitutions, e.g., conserved substitutions).

In certain embodiments, the nucleic acid can comprise a nucleotide sequence encoding at least one, two, or three CDRs or hypervariable loops from a heavy chain variable region having the nucleotide sequence as set forth in Table 1, a sequence substantially homologous thereto (e.g., a sequence having at least about 85%, 90%, 95%, or 99% sequence identity thereto, and/or capable of hybridizing under the stringency conditions described herein). In another embodiment, the nucleic acid can comprise a nucleotide sequence encoding at least one, two, or three CDRs or hypervariable loops from a light chain variable region having the nucleotide sequence as set forth in Table 1, or a sequence substantially homologous thereto (e.g., a sequence having at least about 85%, 90%, 95%, or 99% sequence identity thereto, and/or capable of hybridizing under the stringency conditions described herein). In yet another embodiment, the nucleic acid can comprise a nucleotide sequence encoding at least one, two, three, four, five, or six CDRs or hypervariable loops from heavy and light chain variable regions having the nucleotide sequence as set forth in Table 1, or a sequence substantially homologous thereto (e.g., a sequence having at least about 85%, 90%, 95%, or 99% sequence identity thereto, and/or capable of hybridizing under the stringency conditions described herein).

In another aspect, the application features host cells and vectors containing the nucleic acids described herein. The nucleic acids may be present in a single vector or separate vectors present in the same host cell or separate host cell, as described in more detail herein below.

### Vectors

Further provided herein are vectors comprising nucleotide sequences encoding an antibody molecule described herein. In one embodiment, the vectors comprise nucleotides encoding an antibody molecule described herein. In one embodiment, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity. Methods and conditions for culturing the resulting transfected cells and for recovering the antibody molecule produced are known to those skilled in the art, and may be varied or optimized depending upon the specific expression vector and mammalian host cell employed, based upon the present description.

### Cells

The invention also provides host cells comprising a nucleic acid encoding an antibody molecule as described herein.

In one embodiment, the host cells are genetically engineered to comprise nucleic acids encoding the antibody molecule.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The invention also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells (e.g., CHO-C₈TD cells), HEK293 cells, BHK cells and MDCKII cells. Suitable insect cells include, but are not limited to, Sf9 cells.

### EXAMPLES

The Examples below are set forth to aid in the understanding of the inventions but are not intended to, and should not be construed to limit its scope in any way.

### Example 1: Generation and Characterization of Anti-CD73 Antibodies

Selection and Optimization of Anti-CD73 Antibodies from Synthetic Yeast Antibody Libraries

Anti-CD73 monoclonal antibodies representing five distinct epitope bins were selected from eight naïve human synthetic yeast libraries using methods described below.

### Materials and Methods

Antigens were biotinylated using the EZ-Link Sulfo-NHS-Biotinylation Kit from Pierce. Goat F(ab′)₂ anti-human kappa-FITC (LC-FITC). ExtrAvidin.-PE (EA-PE) and Streptavidin-AF633 (SA-633) were obtained from Southern Biotech, Sigma, and Molecular Probes, respectively. Streptavidin MicroBeads and MACS LC separation columns were purchased from Miltenyi Biotec. Goat anti-human IgG-PE (Human-PE) was obtained from Southern Biotech.

### Primary Discovery

Eight naïve human synthetic yeast libraries each of ˜10⁹ diversity were propagated as previously described (see, e.g., Y. Xu et al, Addressing polyspecificity of antibodies selected from an in vitro yeast presentation system: a FACS-based, high-throughput selection and analytical tool. PEDS 26.10, 663-70 (2013); WO2009036379; WO2010105256; and WO2012009568, incorporated by reference herein in their entireties). For the first two rounds of selection, a magnetic bead sorting technique utilizing the Miltenyi MACS system was performed, as previously described (see, e.g., Siegel et al, h efficiency recovery and epitope-specific sorting of an scFv yeast display library. J Immunol Methods 286(1-2), 141-153 (2004), herein incorporated by reference in its entirety). Briefly, yeast cells (˜10¹⁰ cells/library) were incubated with 3 ml of 100 nM biotinylated antigen for 30 min at 30° C. in wash buffer (phosphate-buffered saline (PBS)/0.1% bovine serum albumin (BSA)). After washing once with 40 ml ice-cold wash buffer, the cell pellet was resuspended in 20 mL, wash buffer, and Streptavidin MicroBeads (500 μl) were added to the yeast and incubated for 15 min at 4° C. Next, the yeast cells were pelleted, resuspended in 20 mL wash buffer, and loaded onto a Miltenyi LS column. After the 20 mL was loaded, the column was washed 3 times with 3 ml wash buffer. The column was then removed from the magnetic field, and the yeast cells were eluted with 5 mL of growth media and then grown overnight. The following rounds of selection were performed using flow cytometry. Approximately 2×10⁷ yeast cells were pelleted, washed three times with wash buffer, and incubated at 30° C. with either decreasing concentrations of biotinylated antigen (100 to 1 nM) under equilibrium conditions, 30 nM biotinylated antigens of different species in order to obtain species cross-reactivity, or with a poly-specificity depletion reagent (PSR) to remove non-specific antibodies from the selection. For the PSR depletion, the libraries were incubated with a 1:10 dilution of biotinylated PSR reagent as previously described (see, e.g., Y. Xu et al, Addressing polyspecificity of antibodies selected from an in vitro yeast presentation system: a FACS-based, high-throughput selection and analytical tool. PEDS 26.10, 663-70 (2013), herein incorporated by reference in its entirety). Yeast cells were then washed twice with wash buffer and stained with LC-FITC (diluted 1:100) and either SA-633 (diluted 1:500) or EAPE (diluted 1:50) secondary reagents for 15 min at 4° C. After washing twice with wash buffer, the cell pellets were resuspended in 0.3 mL wash buffer and transferred to strainer-capped sort tubes. Sorting was performed using a FACS ARIA sorter (BD Biosciences) and sort gates were determined to select for antibodies with desired characteristics. Selection rounds were repeated until a population with all of the desired characteristics was obtained. After the final round of sorting, yeast cells were plated and individual colonies were picked for characterization.

Light chain diversification protocol was used during the primary discovery phase for further discovery and improvement of antibodies.

Light chain batch diversification protocol: Heavy chain plasmids from a naïve selection output were extracted from the yeast via smash and grab, propagated in and subsequently purified from E. coli, and transformed into a light chain library with a diversity of 5×10⁶. Selections were performed with one round of MACS and four rounds of FACS employing the same conditions as the naïve discovery.

### Antibody Optimization

Optimization of antibodies was performed by introducing diversities into the heavy chain and light chain variable regions as described below.

CDRH1 and CDRH2 selection: The CDRH3 of a single antibody was recombined into a premade library with CDRH1 and CDRH2 variants of a diversity of 1×10⁸ and selections were performed with one round of MACS and four rounds of FACS as described in the naïve discovery. In the different FACS rounds the libraries were looked, at for PSR binding, species cross-reactivity, and affinity pressure by titration or parental Fab pre-complexing, and sorting was performed in order to obtain a population with the desired characteristics.

### Antibody Production and Purification

Yeast clones were grown to saturation and then induced for 48 h at 30° C. with shaking. After induction, yeast cells were pelletal and the supernatants were harvested for purification. IgGs were purified using a Protein A column and eluted with acetic acid, pH 2.0. Fab fragments were generated by papain digestion and purified over KappaSelect (GE Healthcare LifeSciences).

### ForteBio K_{D} Measurements

ForteBio affinity measurements were performed on an Octet RED384 generally as previously described (see, e.g., Estep et al, High throughput solution-based measurement of antibody-antigen affinity and epitope binning Mabs 5(2), 270-278 (2013), herein incorporated by reference in its entirety). Briefly, ForteBio affinity measurements were performed by loading IgGs on-line onto AHQ sensors. Sensors were equilibrated off-line in assay buffer for 30 min and then monitored on-line for 60 seconds for baseline establishment. Sensors with loaded IgGs were exposed to 100 nM antigen for 3 minutes, and afterwards were transferred to assay buffer for 3 min for off-rate measurement. All kinetics were analyzed using the 1:1 binding model. Antigens used were:
Human CD73-His: Recombinant Human 5′-Nucleotidase/CD73 Protein, CF from R&D Systems Cat: 5795-EN
Mouse CD73-His: Recombinant Mouse 5′-Nucleotidase/CD73 Protein, CF from R&D Systems

Cat: 4488-EN
Cynomolgus CD73-His: Cynomolgus CD73/NTSE Protein (His Tag) from Sino Biological Cat: 90192-CO8H-50

### ForteBio Epitope Binning/Ligand Blocking

Epitope binning/ligand blocking was performed using a standard sandwich format cross-blocking assay. Control anti-target IgG was loaded onto AHQ sensors and unoccupied Fc-binding sites on the sensor were blocked with an irrelevant human IgG1 antibody. The sensors were then exposed to 100 nM target antigen followed by a second anti-target antibody or ligand. Additional binding by the second antibody or ligand after antigen association indicates an unoccupied epitope (non-competitor), while no binding indicates epitope blocking (competitor or ligand blocking).

### MSD-SET Kinetic Assay

Equilibrium affinity measurements performed as previously described (Estep et at, 2013). Solution equilibrium titrations (SET) were performed in PBS+0.1% IgG-Free BSA (PBSF) with antigen held constant at 10-100 pM and incubated with 3- to 5-fold serial dilutions of antibody starting at 5-100 nM (experimental condition is sample dependent). Antibodies (20 nM in PBS) were coated onto standard bind MSD-ECL plates overnight at 4° C. or at room temperature for 30 min. Plates were then blocked for 30 min with shaking at 700 rpm, followed by three washes with wash buffer (PBSF+0.05% Tween 20). SET samples were applied and incubated on the plates for 150s with shaking at 700 rpm followed by one wash. Antigen captured on a plate was detected with 250 ng/mL sulfotag-labeled streptavidin in PBSF by incubation on the plate for 3 min. The plates were washed three times with wash buffer and then read on the MSD Sector Imager 2400 instrument using 1× Read Buffer T with surfactant. The percent free antigen was plotted as a function of titrated antibody in Prism and fit to a quadratic equation to extract the K_{D}. To improve throughput, liquid handling robots were used throughout MSD-SET experiments, including SET sample preparation.

### Cell Binding Analysis

Approximately 100,000 cells overexpressing the antigen were washed with wash buffer and incubated with 100 μl 100 nM IgG for 5 minutes at room temperature. Cells were then washed twice with wash buffer and incubated with 100 μl of 1:100 Human-PE for 15 minutes on ice. Cells were then washed twice with wash buffer and analyzed on a FACS Canto II analyzer (BD Biosciences).

### Results

Yeast cells expressing a library of human antibodies on their surface were screened for binding to human CD73. Two antibodies from epitope bin 4, 918 and 930, bound well to CD73 and inhibited the enzymatic activity of CD73 (data not shown). These two antibodies were subjected to affinity maturation which produced two lineages of related antibodies, referred to as lineage 1 and lineage 3, respectively (Table 19). These anti-CD73 antibodies were expressed in three different formats: IgG1 antibodies (referred to as 0.0 constructs, e.g., 350.C), IgG4 antibodies comprising an S228P mutation in the Fc region (IgG4 S228P, referred to as .A constructs, e.g., 350.A), or IgG4 antibodies comprising S228P and L235E mutations in the Fc region (IgG4 S228P/L235E, referred to as .B constructs, e.g., 350.B), numbered according to Eu numbering. The sequences of these antibodies are disclosed in Table 1. For the antibody 350.A, two lots of antibodies were produced, referred to hereafter as 350.A1 and 350.A2.

TABLE 19
Two lineages of anti-CD73 antibodies

| | | | IgG4 S228P | IgG4 S228P/ |
|---|---|---|---|---|
| | Antibody | IgG1 format | format | L235E format |
| Lineage 1 | | | | |
| Parent | 918 | 918.C | 918.A | 918.B |
| Child | 350 | 350.C | 350.A1, 350.A2 | 350.B |
| Child | 356 | 356.C | 356.A | 356.B |
| Child | 358 | 358.C | 358.A | 358.B |
| Lineage 3 | | | | |
| Parent | 930 | 930.C | 930.A | 930.B |
| Child | 373 | 373.C | 373.A | 373.B |
| Child | 374 | 374.C | 374.A | 374.B |
| Child | 376 | 376.C | 376.A | 376.B |
| Child | 377 | 377.C | 377.A | 377.B |
| Child | 379 | 379.C | 379.A | 379.B |

All the anti-CD73 antibodies tested bind to human and cynomolgus CD73. The Lineage 1 antibodies also bind to murine CD73. Table 20 provides K_{d} values of these antibodies measured using Octet as described above.

TABLE 20
Affinities of anti-CD73 antibodies

| | Human ForteBio | Cyno ForteBio | Murine ForteBio |
|---|---|---|---|
| | Whole | Whole | Whole |
| mAb | IgG Kd (M) | IgG Kd (M) | IgG Kd (M) |
| 918.C | 1.53E−09 | Not Determined | 8.88E−09 |
| 350.C | 2.48E−10* | 6.35E−10 | 4.09E−10* |
| 356.C | 2.43E−10* | 6.93E−10 | 4.01E−10 |
| 358.C | 3.17E−10 | 8.14E−10 | 3.51E−09 |
| 930.C | 1.83E−09 | Not Determined | Not Bound |
| 373.C | 6.21E−10 | 9.14E−10 | Not Bound |
| 374.C | 3.58E−10 | 1.03E−10 | Not Bound |
| 376.C | 3.01E−10 | Not Determined | Not Bound |
| 377.C | 5.72E−10 | 1.42E−09 | Not Bound |
| 379.C | 5.50E−10 | 1.62E−09 | Not Bound |

*Value approaching upper limit for K_{d} measurement

Next, using epitope binning/ligand blocking studies, it was shown that the parental antibody 918 competed for binding to CD73 with the progeny antibodies 350, 356, and 358. Similarly, the parental antibody 930 competed for binding to CD73 with the progeny antibodies 373, 374, 376, 377, and 379. Both 918 and 930 were shown to compete with an internal reference anti-CD73 antibody, suggesting that these antibodies share the same epitope bin.

### Fab and Antibody Affinity Measurement Using Surface Plasmon Resonance

Fabs of mAbs 350 and 373 were generated by engineering a stop between the two proline residues above the core hinge region of the heavy chain of 350 and 373. Both were expressed in Expi293F (ThermoFisher) cells and purified using CaptureSelect IgG CH₁ Affinity resin (ThermoFisher).

Biacore was used to measure cross-species affinity for the Fab materials of mAbs 350 and 373. Proteins used were as follows: recombinant human CD73 (R&D Systems 5795-EN); recombinant cynomolgus monkey CD73 (Sino Biological 90912-C08H); recombinant mouse CD73 (R&D Systems 4488-EN); and recombinant rat CD73 (Sino Biological 80375-R08H). Anti-human Fab (GE Healthcare Life Sciences) was immobilized on all 4 flow cells (Fc) on a CMS chip (GE). Fabs 350 and 373 were captured on Fc2 and Fc4, at ˜20 RU. 0.01 nM to 90 nM CD73 (3-fold dilution series) was flown over all 4 Fcs. All samples were diluted in running buffer HBS-EP+ (pH 7.4, 0.01 M HEPES, 150 mM NaCl, 3 mM EDTA and 0.05% (v/v) P20).

Shown in Table 21 are results for K_{d} (M) affinity for cross-species binding of 350 and 373 Fabs.

TABLE 21
Affinities of anti-CD73 Fabs

| Fab | Antigen | K_{d} (M) |
|---|---|---|
| Fab 350 | hCD73 | ≤1E−10 |
| Fab 350 | cCD73 | ≤1E−10 |
| Fab 350 | mCD73 | 1.728E−8 |
| Fab 350 | rCD73 | 2.829E−8 |
| Fab 373 | hCD73 | 1.304E−8 |
| Fab 373 | cCD73 | 9.465E−9 |
| Fab 373 | mCD73 | No Binding |
| Fab 373 | rCD73 | No Binding |

In a separate study, the affinity of the full-length antibody 373.A or Fab fragments of 373.A to human, cynomolgus monkey, mouse and rat CD73 was determined using an anti-histidine (His) antibody capture Biacore method utilizing surface plasmon resonance (SPR). The anti-His Ab was directly immobilized onto a CM5 chip surface by amine coupling. The His-tagged human CD73/His, cynomolgus monkey CD73/His, mouse CD73/His or rat CD73/His was flowed over and captured at a desired resonance unit (RU) for an Rmax of 20. Antibody analyte concentrations in serial dilutions of IgG or Fab were flowed over at 60 μL/min. The sensorgrams were analyzed using the manufacturer's software for a 1:1 binding model. Binding to mouse CD73/His protein and rat CD73/His protein was undetectable for 373.A and 373.A Fab, demonstrating that 373.A is not rodent cross-reactive.

Affinities were established for human and cynomolgus monkey CD73 with both 373.A and 373.A Fab. Hydrogen-deuterium mass spectrometry and size exclusion chromatography studies support a model of conformational locking of the CD73-dimer by 373.A into the open-open (inactive-inactive) conformation, supporting a 1:1 bidentate binding of 1 Ab:1 CD73 dimer (see Example 2). Therefore, given that 1:1 bidentate binding will favor avidity, the whole Ab affinities were used rather than Fab measurements. The full length antibody 373.A binds recombinant human CD73 with a K_{d} of 0.991±0.267 nM and cross-reacts with recombinant cynomolgus monkey CD73 with a K_{d} of 0.068±0.009 nM as determined by Biacore kinetic binding studies.

### Whole Blood Target Engagement by Anti-CD73 Antibodies

Whole blood target engagement was assessed by flow cytometry using whole blood from healthy human donors. Briefly, biotinylated antibodies were incubated with whole blood for 30 minutes prior to red blood cell lysis and fixation. Fixed cells were stained for CD3 and CD8 to identify CD8+ T cells, and streptavidin-APC to detect biotin. After staining, the cell were washed and subjected to flow cytometry analysis.

Dose dependent binding, as measured by median fluorescence intensity (MFI) of APC signal, was observed for the anti-CD73 antibodies tested (FIG. 1). A biotinylated isotype control antibody did not show binding to CD8+ T cells (FIG. 1).

### CD73 Target Occupancy on Human Whole Blood Samples

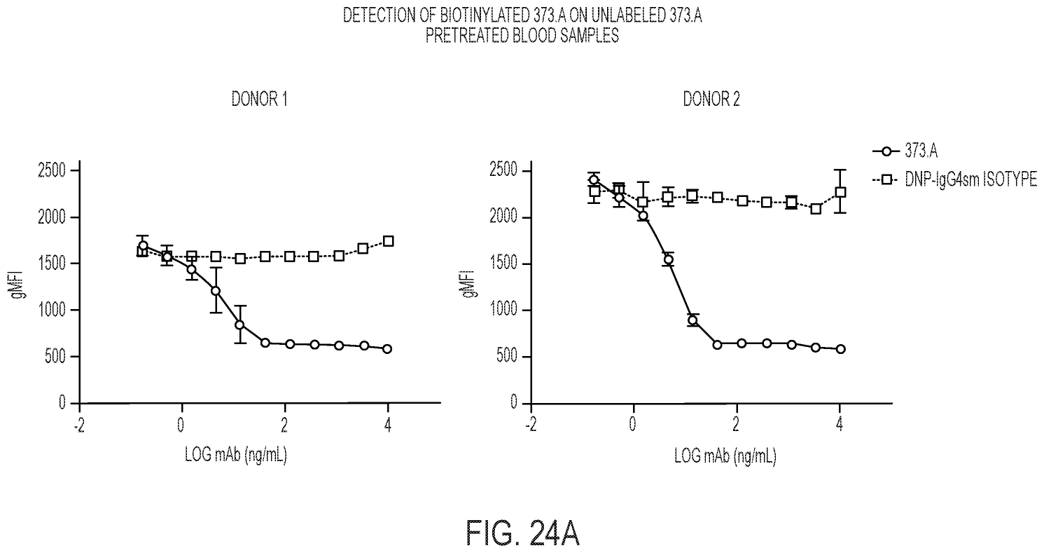

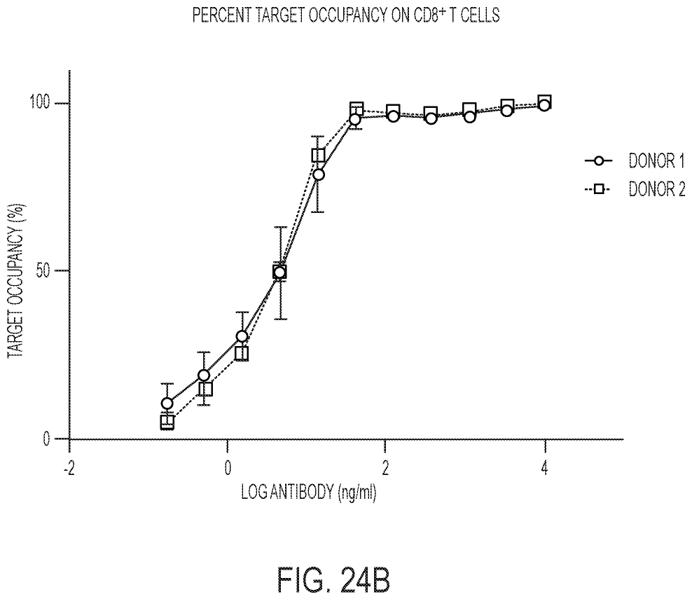

Conceptual demonstration of the target occupancy (TO) of CD73 on human whole blood samples was performed by treating donor blood ex vivo with unlabeled 373.A. A titration of 373.A or DNP-IgG4sm isotype control from 10 μg/mL to 0.17 ng/mL was performed. As shown in FIG. 24A, samples from the two donors that were treated with unlabeled 373.A at higher doses (10 μg/mL to ˜0.1 μg/mL) prevented biotinylated 373.A from binding to the cells, reducing the geometric mean fluorescence intensity (gMFI) values to a plateau at the background level of fluorescence (˜-550 gMFI for both donors). This is indicative of full CD73 target occupancy. In contrast, gMFI values for cells that were pretreated with lower amounts of unlabeled 373.A (0.17 ng/mL and 0.51 ng/mL) were similar to samples that were pretreated with DNP-IgG4sm isotype control (˜1600 gMFI for donor 1 and ˜2200 gMFI for donor 2). Isotype control treated samples mimicked blood that had zero target occupancy. The resultant % TO values are shown in FIG. 24B.

### Inhibition of the Enzymatic Activity of Soluble Recombinant CD73

5′ ectonucleotidase CD73 is the rate limiting step in the conversion of AMP to adenosine. The ability of anti-CD73 antibodies to inhibit the enzymatic activity of CD73 was measured using a malachite green phosphate assay. Briefly, 25 ng/ml recombinant human CD73 was incubated with a dose titration of the substrate adenosine monophosphate (AMP) (0-500 μM) with buffer alone, or in the presence of an isotype control antibody at 1 μg/ml or the anti-CD73 antibody 350.0 at 1, 0.3, or 0.1 μg/ml. Release of inorganic phosphate (Pi) was measured using a malachite green phosphate assay kit (Enzo Life Sciences, Catalog #BML-AK111).

As shown in FIG. 2A, the control antibody at the tested concentration had no effect on the Michaelis constant (Kₘ) of recombinant human CD73. In contrast, the anti-CD73 antibody 350.0 caused dose-dependent reduction of Vₘₐₓ on Kₘ curves (FIG. 2B), indicating that the antibody 350.0 is a non-competitive inhibitor of human CD73.

Next, the anti-CD73 antibodies 350, 356, 373, and 374, expressed in either the .A or .B format, were tested for their ability to inhibit the enzymatic activity of recombinant human and cynomolgus monkey CD73 using a similar malachite green phosphate assay as described above. In brief, anti-CD73 antibodies were incubated for 10 minutes with 25 ng/ml recombinant human or cynomolgus CD73 in the presence of 25 μM AMP. Release of inorganic phosphate (Pi) was measured using a malachite green phosphate assay kit (Enzo Life Sciences, Catalog #BML-AK111). Normalized percent inhibition (% INH) was determined using time zero control as 100% INH and no antibody control as 0% INH.

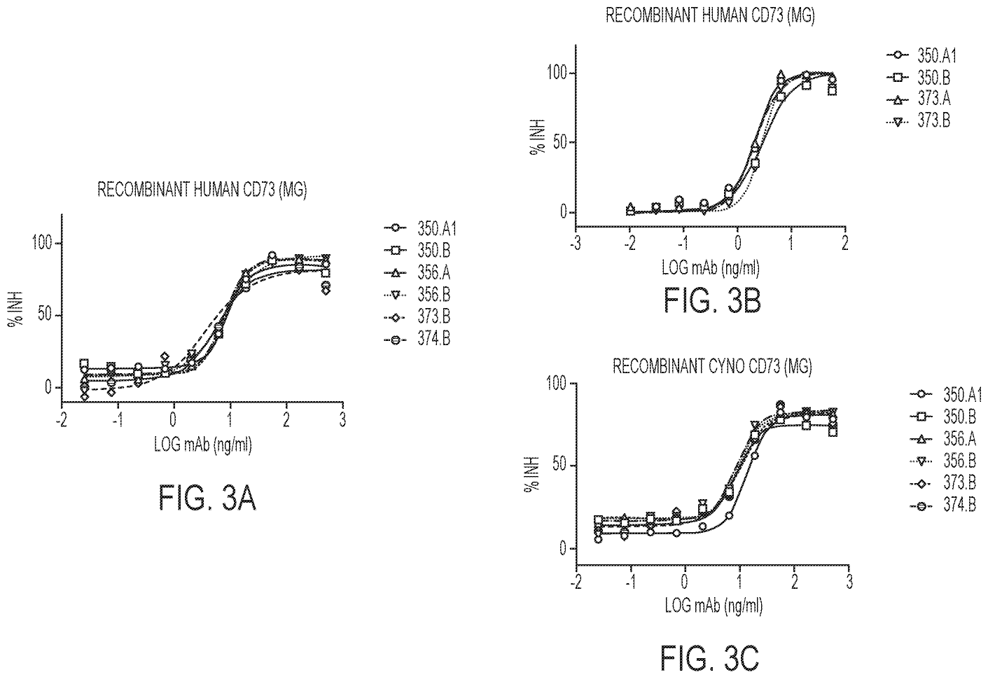

As shown in FIGS. 3A-3C, all the anti-CD73 antibodies tested inhibited the enzymatic activity of soluble recombinant human and cynomolgus CD73.

### Inhibition of the Enzymatic Activity of Soluble Endogenous CD73

Further, the enzyme inhibition activity of anti-CD73 antibodies was tested against soluble endogenous CD73, for example, CD73 shed from the cell surface.

In a first study, anti-CD73 antibodies 350 and 373, expressed in either the .A or .B format, or isotype control antibodies were incubated for 240 minutes with MDA-MB-231 (a human breast cancer cell line) conditioned serum free media in the presence of 100 μM AMP. Disappearance of AMP was measured by a modified Cell Titer Glo (CTG) assay (Promega, Cat #G9242/3). AMP inhibits the luciferase signal in the CTG kit. The luciferase signal increases as the added AMP is enzymatically consumed by CD73. Normalized percent inhibition (% INH) was determined using time zero control as 100% INH and no antibody control as 0% INH.

As shown in FIG. 4, the anti-CD73 antibodies dose-dependently inhibited the enzymatic activity of CD73 shed from the breast cancer cell line MDA-MB-231.

In a second study, anti-CD73 antibodies 350, 356, 358, 373, 374, 377, and 379, all expressed in the .B format, were incubated for 60 minutes with diluted (12.5% v:v in PBS) serum from a pancreatic cancer patient in the presence of 100 μM AMP. Similar to the first study, disappearance of AMP was measured by the modified Cell Titer Glo (CTG) assay and normalized percent inhibition (% INH) was determined using time zero control as 100% INH and no antibody control as 0% INH.

Anti-CD73 antibodies also inhibited CD73 enzymatic activity in the serum from the pancreatic cancer patient in a dose-dependent manner (FIG. 5).

### Inhibition of the Enzymatic Activity of CD73 Expressed on the Cell Surface

First, a malachite green phosphate assay was used to examine the ability of anti-CD73 antibodies 350, 356, 358, 373, 374, 377, and 379 (all in the .B format) to inhibit CD73 expressed on a breast cancer cell line MDA-MB-231. Briefly, antibodies were incubated for 180 minutes with cells in the presence of 100 μM AMP. Release of inorganic phosphate from AMP was measured using a malachite green phosphate assay kit (Enzo Life Sciences, Catalog #BML-AK111). Normalized percent inhibition (% INH) was determined using time zero control as 100% INH and no antibody control as 0% INH.

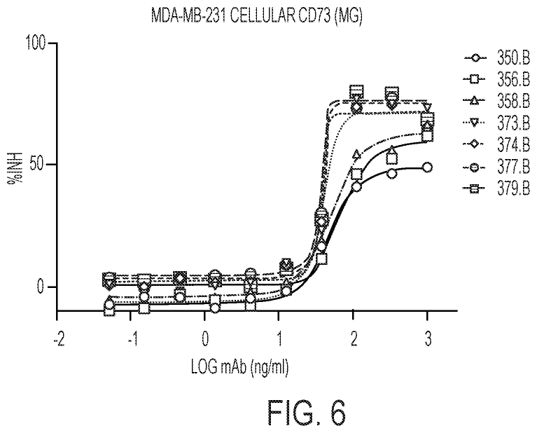

As shown in FIG. 6, all the anti-CD73 antibodies tested inhibited CD73 enzymatic activity expressed on the surface of the breast cancer cell line MDA-MB-231.

Next, since the Lineage 1 antibodies cross-react with mouse CD73 whereas the Lineage 3 antibodies do not, antibodies from both lineages were tested against CD73 expressed on the surface of a human or murine breast cancer cell line. Anti-CD73 antibodies were incubated for 240 minutes with a human breast cancer cell line MDA-MB-231 or a murine breast cancer cell line 4T1 in the presence of 100 μM AMP. Disappearance of AMP was measured by the modified Cell Titer Glo (CTG) assay described above and normalized percent inhibition (% INH) was determined using time zero control as 100% INH and no antibody control as 0% INH.

Consistent with their binding profiles, the Lineage 1 antibodies 918, 350, 356, and 358 inhibited both human and murine CD73 (FIGS. 7A and 7B), whereas the Lineage 3 antibodies 930, 373, 374, 376, 377, and 379 inhibited human, but not murine, CD73 (FIGS. 7C and 7D).

Furthermore, two modified Cell Titer Glo (CTG) assays were conducted to test the enzyme inhibition activity of anti-CD73 antibodies against CD73 expressed on a human breast cancer cell line

MDA-MB-231 or a human ovarian cancer cell line SKOV3. In both studies, 1000 ng/ml anti-CD73 antibodies were incubated for 240 minutes with 20,000 cells/ml cells in the presence of 100 μM AMP at 37° C. Disappearance of AMP was measured by the modified Cell Titer Glo (CTG) assay described above and normalized percent inhibition (% INH) was determined using time zero control as 100% INH and no antibody control as 0% INH.

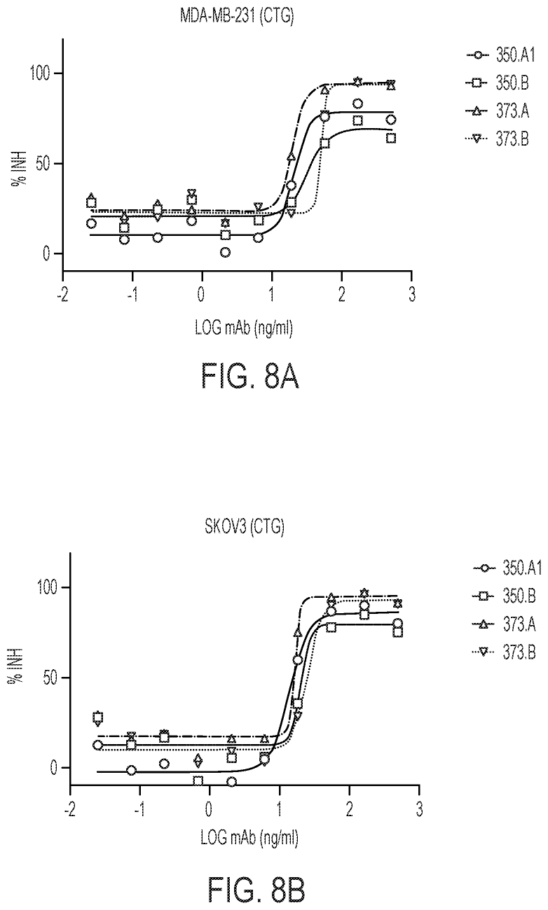

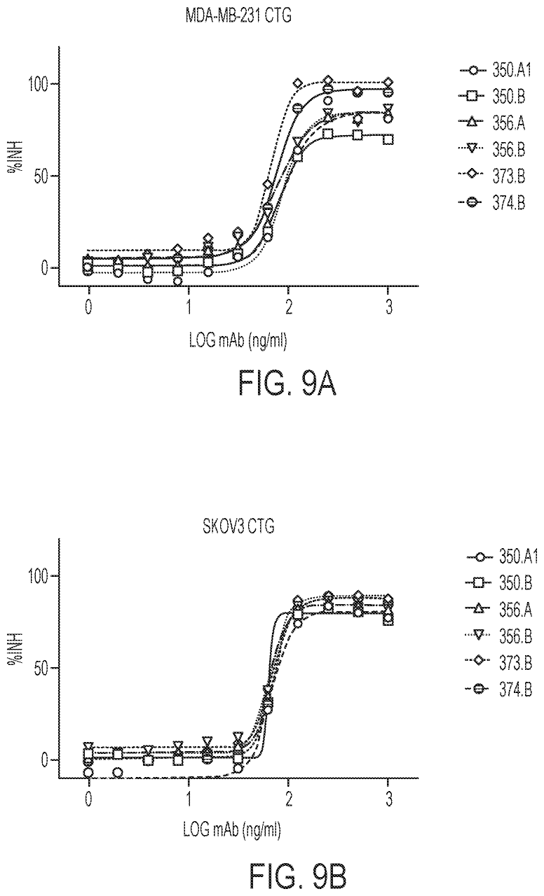

In both studies, all the anti-CD73 antibodies tested were able to inhibit surface CD73 expressed on the human breast cancer cell line MDA-MB-231 or the human ovarian cancer cell line SKOV3 (FIGS. 8A, 8B, 9A, and 9B).

Next, a similar Cell Titer Glo (CTG) assay was performed to examine the ability of anti-CD73 antibodies to inhibit human CD73 expressed on a HEK 293 cell line. Briefly, a HEK 293 cell line was engineered to stably overexpress human CD73 and incubated with anti-CD73 antibodies for 150 minutes in the presence of 100 μM AMP. Disappearance of AMP was measured by the modified Cell Titer Glo (CTG) assay described above and normalized percent inhibition (% INH) was determined using time zero control as 100% INH and no antibody control as 0% INH.

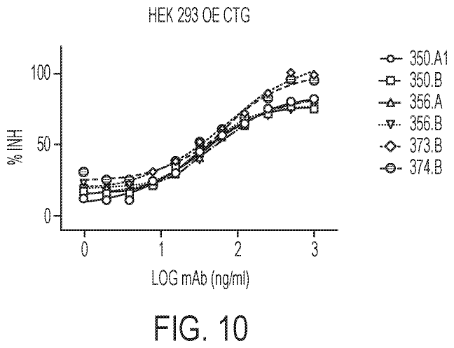

As shown in FIG. 10, the anti-CD73 antibodies 350, 356, 373, and 374, in the .A or .B format, inhibited membrane-bound human CD73 in a dose-dependent manner

In addition, the enzyme inhibition activity of anti-CD73 antibodies were also examined using human PBMCs. In brief, primary human PBMCs were isolated from two separate donors and incubated with anti-CD73 antibodies for 480 minutes in the presence of 25 μM AMP. Disappearance of AMP was measured by the modified Cell Titer Glo (CTG) assay described above and normalized percent inhibition (% INH) was determined using time zero control as 100% INH and no antibody control as 0% INH.

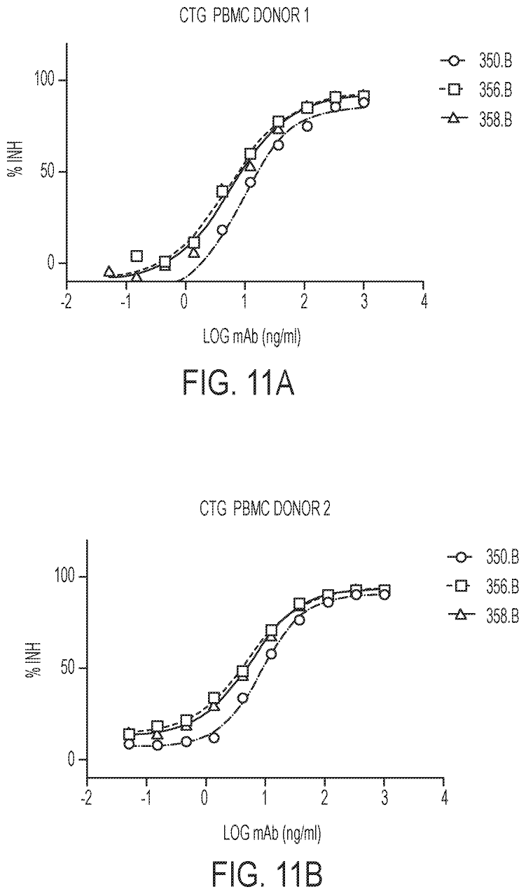

As shown in FIGS. 11A and 11B, the anti-CD73 antibodies tested inhibited the enzymatic activity of CD73 expressed on primary human PBMCs from both donors.

### Restoration of CD4+ and CD8+ T Cell Proliferation in the Presence of AMP

Next, anti-CD73 antibodies were tested for their ability to relieve AMP-mediated inhibition of CD4+ T cells. Briefly, CD4+ T cells were isolated from healthy human donor pooled Peripheral Blood Mononuclear Cells (PBMC). Prior to stimulation with anti-CD3/28 beads in the presence of 800 μM AMP, CD4+ T cells were stained with CellTrace Violet (CTV) (Thermo Fisher Scientific, Cat #C34557) to track cell division. On day 4, proliferation was determined by CTV dilution using flow cytometry. Cells stained with CTV lose approximately half of their fluorescence signal as measured on the flow cytometer with each division. Proliferation index was calculated as a measure of the level of T cell division for each condition where 100 represents maximal proliferation and 0 represents no proliferation.

As shown in FIGS. 12A and 12B, all the anti-CD73 antibodies tested were able to restore CD4+ T cell proliferation in the presence of AMP.

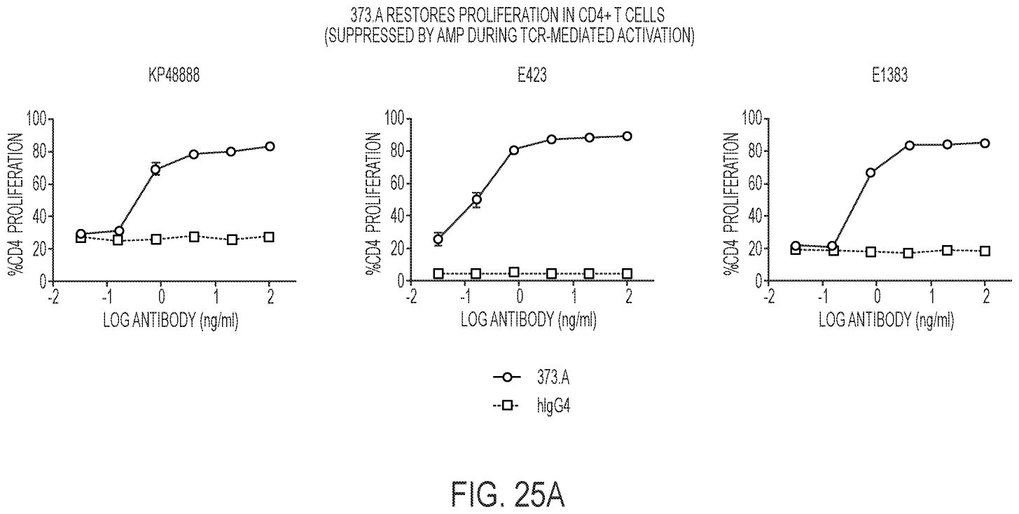

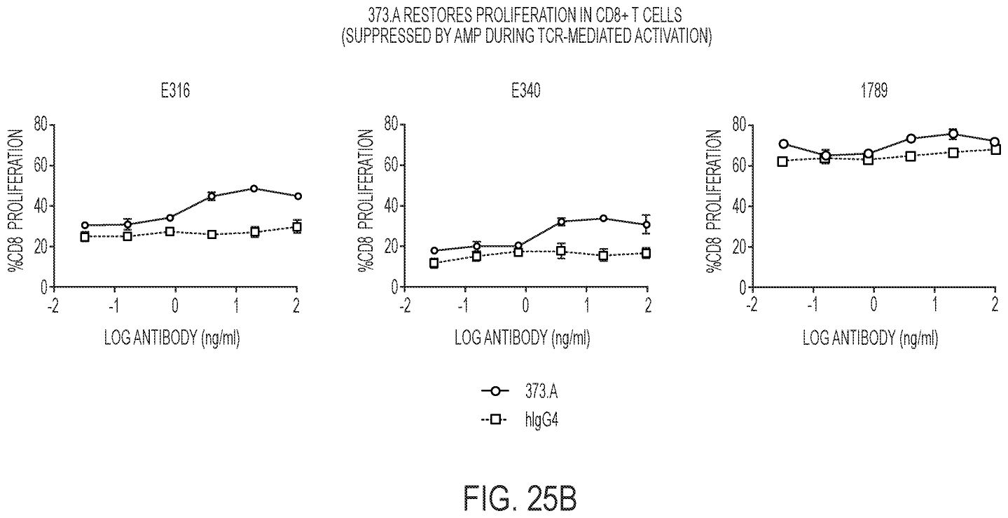

In a separate study, both CD4+ and CD8+ T cells were tested. Briefly, CD4+ and CD8+ T cells were purified from peripheral blood mononuclear cells (PBMC) isolated from healthy human donors and labeled with carboxyfluorescein succinimidyl ester (CFSE). CFSE-labeled CD4+ or CD8+ T cells were activated for 4 days with anti-CD3/CD28 T cell activator beads (1 bead/16 cells) in the presence of 800 □M AMP and serial dilutions of either isotype control hIgG4 or 373.A. Representative data from three donors each are shown for CD4+ T cells (FIG. 25A) and CD8+ T cells (FIG. 25B). Data are represented as percent CD4+ (FIG. 25A) or CD8+ (FIG. 25B) T cell proliferation where isotype control demonstrates that maximal proliferation is suppressed in the presence of AMP, and the presence of 373.A restores proliferation in a dose-dependent manner. While the magnitude of the response varies across donors, in all three CD4+ T cell donors represented in (FIG. 25A) and 2/3 donors shown for CD8+ T cells (FIG. 25B), 373.A demonstrates in vitro activity in restoring T cell proliferation.

### Inhibition of the Enzymatic Activity of CD73 In Vivo

Furthermore, anti-CD73 antibodies were examined for their enzyme inhibition activity in vivo. Athymic nude, female mice (6-8 weeks of age) were implanted with high CD73-expressing MDA-MB231 breast cancer cell line (ATCC HTB-26) at 10×10⁶ cells/mouse/200 μl. Five mice per group were randomized when tumors were 200 mm³ and treated intraperitoneally with either 20 or 200 μg/mouse of control polyclonal human IgG or a panel of anti-CD73 mAbs. The antibodies tested are the anti-CD73 antibodies 350, 356, 373, and 374, expressed in either the .A or .B format.

Plasma was collected three days post-dose at a ratio of one portion of plasma into five portions of methanol by volume. The methanol quenched samples were stored at −80° C. before use, at which time the samples were centrifuged. The precipitations were discarded and the supernatants were transferred to new Eppendorf tubes. Stock solutions of internal standards (IS, C-13 labeled adenosine and N-15 labeled Inosine, Cambridge Isotope Laboratories, MA) were added to the final concentration of 50 nM. The prepared samples were then analyzed using an LC/MS system of API-6500 QTrap (AB Sciex, US) coupled with a Shimadzu LC pump (LC-20AD) and a CTC auto sampler with DLW wash. For each sample, 5 μL was injected and separated using a SeQuant ZIC-pHILIC column (5 μm, 150×2.1 mm, Millipore, Mass.) maintained at 40° C. A binary gradient was used for the elution, where mobile phase B is 100% acetonitrile with no additives and mobile phase A is 12 mM ammonium formate and 12 mM formic acid in 1:1 (v/v) mix of water and acetonitrile. The elution was programed as (0, 85, 0.6), (0.5, 85, 0.4), (2, 10, 0.4), (4.5, 10, 0.4), (5, 85, 0.4), (5.5, 85, 0.6), where values in the parentheses are time in minutes, percent of mobile phase B and flow rate in mL/min in order. Adenosine and C13-Adenosine were monitored from 0.5 to 4.5 minutes at ESI positive mode and mass transitions 268->136 and 273->136 respectively. Inosine and N15-Inosine were monitored from 0.5 to 4.5 minutes at ESI negative mode and mass transitions 267->135 and 271->139 respectively. Results were reported as nM adenosine or inosine.

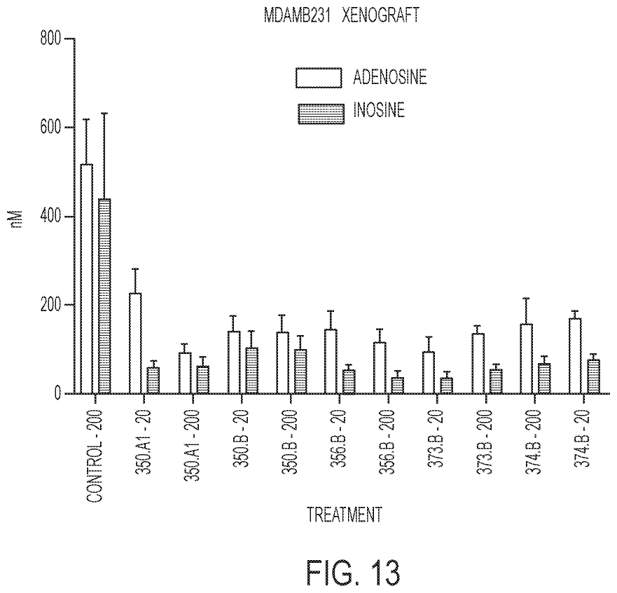

All the anti-CD73 antibodies tested effectively reduced the accumulation of adenosine and inosine in the serum of immunocompromised mice implanted with the high CD73-expressing MDA-MB231 breast cancer cell line (FIG. 13).

### Example 2: Determination of the Epitope and Binding Mode of Anti-CD73 Antibodies

In this example, the epitope and binding mode of anti-CD73 antibodies 350.A2, 350.B, 373.A, and 373.B were determined by fragmentation amide hydrogen/deuterium exchange (HDx) and size exclusion chromatography (SEC).

### Methods

Fragmentation Hydrogen Deuterium-Exchange Mass Spectrometry (HDx-MS)

HDx-MS experiments were performed as previously described (Park I H, et al., J. Chem. Inf. Model; 55(9): 1914-1925 (2015); Chalmers M J, et al., Anal. Chem.; 78(4): 1005-14 (2006)). Antibody antigen complexes were prepared and used in a 1:1 molar ratio by overnight incubation at 4° C. It is important to note that this means that there is a 2-fold excess of Fab binding sites present in the complex mixtures.

Room temperature on-exchange experiments were performed by manual addition of 50 μL of in-exchange buffer (50 mM phosphate, 150 mM NaCl, pH 7.1, in D₂O) to 10 μL of 0.5 mg/ml rhCD73 protein (R&D systems, Catalog #5795, Trp27-Lys547 of AAH65937 fused to a C-terminal 6-His tag) or a molar equivalent amount of CD73-mAb complex (50 mM phosphate, 150 mM NaCl, pH 7.1). Samples were quenched after 60s of in-exchange at room temperature by addition of 250 μL quench buffer (4M guanidinium hydrochloride, 0.5M Tris(2-carboxyethyl)phosphine hydrochloride (TCEP-HCl), 0.2M phosphate, pH 2.5, 0° C.) and after 30s further diluted with 300 μL of ice cold storage buffer (20% glycerol, 0.25% formic acid in water) before flash freezing with liquid nitrogen and storage at −80° C. until use or transfer into a −70° C. drawer attached to the rail of a liquid handler (PAL HTS, LEAP Technologies, Carrboro, N.C.) located inside a 0° C. enclosure. The frozen samples were thawed for 120s by flowing N2 gas over the sample vial (facilitated by the liquid handler in conjunction with a thaw fixture) and loaded into the sample loop of the injection valve (Valco, Houston, Tex.). Loading of samples (load buffer 0.05% Trifluoroacetic acid (TFA), 500 μL/min) onto the pre-column of the chromatographic system and online pepsin digestion (immobilized pepsin on Poros AL20, 2.1×150 mm, maintained at 15° C.) was performed with a dual pump system (2×, Accela 1250, Thermo Scientific, Waltham Mass.) that allowed admixing of a second flow of 550 μL/min (0.05% TFA, 500 μL/min) of load buffer post digestion through a mixing tee. The combined flow was directed into the chromatographic system maintained at 0° C. for desalting and gradient LC separation at a flow rate of 15 μL/min (Dionex UltiMate 3000, Thermo Scientific, Waltham, Mass.) and concurrent mass spectrometric analysis (QExactive, Thermo Scientific, Waltham, Mass.) of the chromatographic effluent. The chromatographic system consisted of a valve (15kPSI Valco, Houston, Tex.), a 4 μL EXP Halo C₁₈ reversed-phase trap cartridge (Optimize Technologies Inc., Oregon City, Oreg.), and an analytical column (2.1×10 mm ID, Prozap 1.5 μm C18, Grace). Gradient separation was from 0% to 40% B over 20 min followed by 40% to 75% B over 5 min using buffer compositions A: 99.75:0.25% v/v (H20: formic acid) and B: 99.75:0.25% v/v (acetonitrile:formic acid).

MS scans were acquired at a resolution of 70,000 over the m/z range of 350-2000 for MS, and 35,000 for MS/MS. The instrument parameters used for all experiments including spray voltage of 2.5 kV, a maximum injection time of 120 ms, AGC target for MS of 500,000 ions were maintained the same for all runs. Sample were analyzed in triplicate.

Peptide identification was performed by converting raw data to .mgf format using Proteome Discoverer 1.4, searched against the construct sequence using MASCOT 2.4 (Matrix Science, London, UK), and filtered using Scaffold 1.4. Filtered results were imported into HDExaminer (v1.3, Sierra Analytics, Modesto, Calif.) together with raw data files for quantitation of deuteration. Deuteration values were exported into Microsoft Excel for calculation of deuteration differences, and normalization of deuterium incorporation by the number of observable amides (number of residues minus 2, further subtracting the number of prolines in the sequence excluding the N-terminus and penultimate residues) in a peptide. For compression of peptide difference data onto the sequence the normalized difference deuteration values were averaged for each primary sequence residue over the peptide observations in which the respective amide was observable.

Water, deuterium oxide, guanidine hydrochloride, sodium chloride, glycerol, formic acid, trifluoroacetic acid (TFA), trifluoroethanol (TFE), acetonitrile (ACN) were from Sigma Chemical Company (St. Louis, Mo.). Tris-(2-carboxyethyl)phosphine) TCEP was from Gold Biotechnology Inc (St. Louis, Mo.).

### Size Exclusion Chromatography (SEC)

An equimolar amount of rhCD73 (3.2 μL, 1.6 mg/mL) was mixed with mAb (2.6 μL, 5 mg/mL) and allowed to complex overnight at 4° C. Samples were run using an Agilent Autosampler connected to an Agilent 1200 series pump with an Agilent UV detector. The system was controlled by Agilent Chemstation software. Column used was Shodex Protein KW-803 column (8×300 mm ID). Mobile Phase: 90% 2×PBS, 10% isopropanol by volume. Flow rate: 500 μL/min. Injection volume: 8 μL. Detection wavelength: 220 nm. Run time: 30 min.

Antibody antigen complexes were prepared and used in a 1:1 ratio using supplied concentration values by overnight incubation at 4° C. It is important to note that this means that there is a nominally 2-fold excess of Fab binding sites in the complex mixtures.

### Structure Data

Structural data used for interpretation was retrieved from the Protein Data Bank (www.rcsb.org) and consisted of the entries 4H2F, 4H21, and 4H1S. Alignments of the various models and visualization was performed with PyMOL (PyMOL Molecular Graphics System, Version 1.8 Schrödinger, LLC).

### Results

### Hydrogen Exchange Data

The HDx MS protection profiles of 373.A and 373.B as shown in FIG. 14 overlap over the full sequence of CD73. This demonstrates the equivalence of the antibodies in terms of their epitope.

The HDx MS protection profiles of 350.A2 and 350.B as shown in FIG. 15 are in excellent agreement with each other over the full sequence of CD73. This demonstrates the equivalence of the antibodies in terms of their epitope.

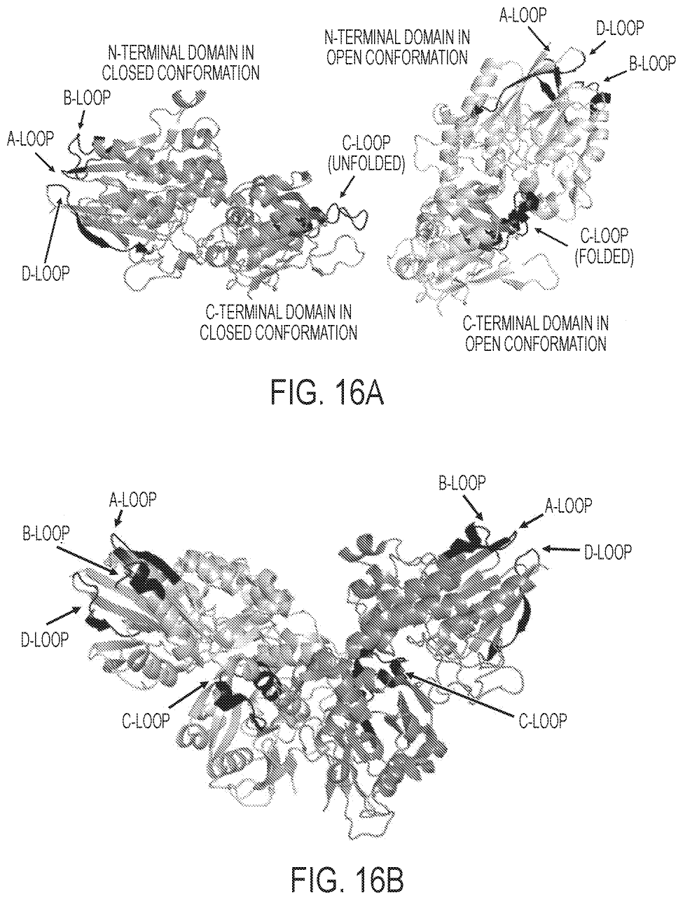

The sequences on which the strongest protection is observed are annotated in the figures and mostly correspond to loop regions of CD73. Their relative spatial arrangement is depicted in the structure models shown in FIGS. 16A and 16B. Whereas protection observed for the sequences of A-, B-, and D-loop is attributable to antibody-CD73 interactions, the protective effects observed for the sequence annotated as C-loop likely arise from conformational locking. The C-loop is folded in the open/inactive conformation of CD73 and unfolded in the closed/active conformation (FIG. 16A). Soluble as well as membrane bound CD73 forms a dimer through the C-terminal domains (FIG. 16B). An equilibrium between open and closed conformations exists as the N-terminal domain rotates by about 90 degrees to convert from open to closed form. The C-loop reports back on that equilibrium. If upon antibody binding to CD73, the equilibrium is shifted more toward the open conformation, a larger fraction of the C-loops will be folded, which is a protected state. On the other hand, if the equilibrium is shifted more toward the closed conformation on antibody binding to CD73, then a deprotective effect is expected. The unusually strong protection observed for 373.A and 373.B at the C-loop (FIG. 14) suggests that these are most efficient in maintaining CD73 in the inactive conformation and/or do so by a distinct mechanism.

From the profiles it is inferred that the maximally protected sequences are residue range 158-172, YLPYKVLPVGDEVVG (SEQ ID NO: 108) assigned as A-loop, residue range 206-215, KLKTLNVNKI (SEQ ID NO: 109) assigned as B-loop, and residue range 368-387, MINNNLRHADETFWNHVSMC (SEQ ID NO: 110) assigned as C-loop in all the figures, numbered according to SEQ ID NO: 105.

For the antibodies 373.A and 373.B, residues in the A- and B-loop regions seem to be equally important for the epitope as indicated by comparable amounts of protection observed in FIG. 14. In the case of the antibodies 350.A2 and 350.B, mostly A-loop residues appear important for the epitope even so some B-loop protection is observed but it is not clear if this should be interpreted as direct engagement of the antibody with residues in that range or is just an indirect effect due to the proximity to the A-loop.

### Size Exclusion Data

The size exclusion data correlate well with the HDx data discussed above. The equivalence of the antibodies 373.A and 373.B is seen in the similarity of their size exclusion chromatography (SEC) profiles, which reflects the oligomerization state distribution (FIGS. 17A and 17B). The same assessment holds for the antibodies 350.A2 and 350.B (FIGS. 18A and 18B).

Features around 17 min correspond to CD73 dimer and free antibody in agreement with the molecular weight scale based on the calibrants, which are shown for reference. The feature at about 14.8 min or about 300 kDa is consistent with a CD73 dimer/mAb complex. Around 12.4 min a feature of approximately 600 kDa consistent with a composition of two CD73 dimers+2 mAbs (2xCD73 dimer/2xmAb) is observed. Additional features above 600 kDa are affected by the exclusion limit of the column. Nevertheless, they can be assigned to higher oligomeric states meaning complexes consisting of 3 or more CD73 dimers in addition to 3 or more mAbs (3⁺xCD73 dimer/3⁺xmAb).

The dominant species in the CD73-373.A and CD73-373.B profiles shown in FIGS. 17A and 17B is the CD73 dimer/mAb complex. Only a minor amount of 2xCD73 dimer/2xmAb species is observed. This is consistent with a preferred bidentate binding mode of one antibody with a CD73 dimer.

The SEC profiles for CD73-350.A2 and CD73-350.B shown in FIGS. 18A and 18B show comparable abundances for the CD73 dimer/mAb species and 2xCD73 dimer/2xmAb species. A shoulder at shorter elution times indicates the presence of some higher oligomers. This shows that 350.A2 and 350.B are capable of binding CD73 like 373.A and 373.B in bidentate fashion but this mode is not necessarily the thermodynamically favored one at least at high concentrations as used in the experiment.

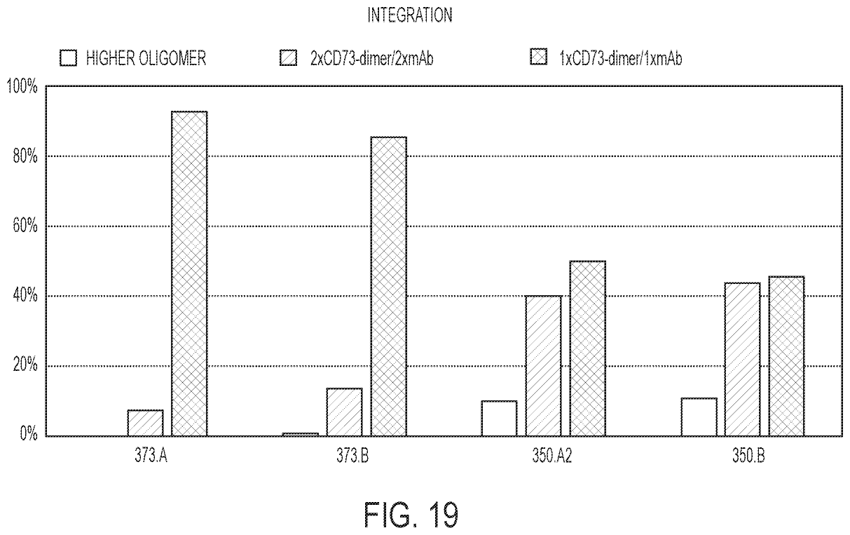

The percentages of anti-CD73 antibodies that formed a 1xCD73 dimer/1xmAb complex, a 2xCD73 dimer/2xmAb complex, or a higher oligomer were estimated by integration of the SEC profiles over the distinguishable species (FIG. 19). A zero baseline was used for all integrations with integration intervals from 10.5 min to 11.65 min for higher oligomers and 11.65 min to 13.5 min for 2xCD73 dimer/2xmAb complex consistently used for all samples. The integration intervals for the 1xCD73 dimer/1xmAb complex species varied slightly and were chosen as 13.5 min to 16.2 min for CD73-373.A, 13.5 min to 16 min for CD73-373.B, 13.5 min to 15.75 min for CD73-350. A2, and 13.5 min to 15.75 min for CD73-350.B to obtain optimal separation from the free antibody signal that was not integrated as it is not relevant for the analysis (antibodies were used in pseudo-excess as about twice the number of Fab binding sites to CD73 binding sites were present in the complex mixtures).

### Conclusion and Discussion

The above HDx and SEC data provide convincing evidence for the equivalence of 373.A and 373.B as well as 350.A2 and 350.B in terms of their mode of interaction with CD73. This was expected considering that the only sequence difference between the pairs is a single mutation (L235E to ablate residual FcR interactions) in the constant region.

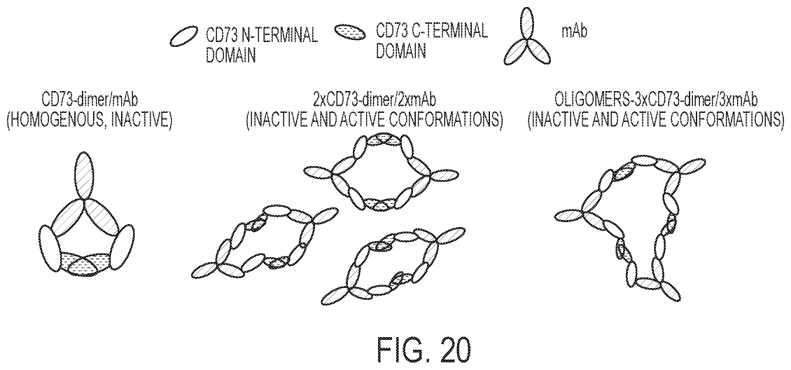

The data from HDx and SEC experiments are consistent and fully support the model presented in FIG. 20. According to the model, the epitopes for all the antibodies are on the N-terminal domains. The antibodies 373.A and 373.B interact with the CD73 dimer to form almost exclusively the homogeneous species illustrated on the left of FIG. 20. In that species, both active sites of the CD73 dimer are locked into the inactive conformation. Further, in this conformation both N-terminal domains of the CD73 dimer are directed away from the cell surface in the case of membrane bound CD73, allowing 373.A and 373.B to inhibit soluble and membrane bound versions of the target with equal efficiency.

The antibodies 350.A2 and 350.B can form the bidentate homogeneous complex on the left but do not show preference for that form over the 2xCD73 dimer/2xmAb species shown in the middle of FIG. 20. Higher oligomeric species illustrated on the right of FIG. 20 are also observed. Presumably the distribution of species will shift toward the homogeneous bidentate complex in the case of membrane bound CD73 or at low concentrations of antibody and antigen, which is the likely scenario in vivo. The antibodies 350.A2 and 350.B are expected to be able to efficiently inhibit all forms of CD73 by the inactive site locking mechanism. It is understood that even in the active conformation, the target could still be catalytically inactive due to antibody binding due to other allosteric inhibition mechanisms.

Based on the model, one would expect that an anti-CD73 antibody that preferentially forms oligomeric species illustrated in the middle and right of FIG. 20 would be less efficient in inhibiting soluble and membrane bound CD73, especially at low concentrations. The reason for this is a large fraction of CD73 will be always in the active conformation and even though the antibody might inhibit catalysis under those circumstances by an indirect mechanism, any detachment of the antibody will immediately produce catalytically active CD73. Further, as total concentration of antigen and antibody go toward zero, two antibody molecules will be required to inhibit a single CD73 dimer, whereas for the 373 and 350 antibodies, a single molecule would still suffice.

### Summary

The putative binding sites of antibodies 373.A, 373.B, 350.A2, and 350.B on rhCD73, as well as their modes of interaction with the rhCD73 dimer were determined. The epitopes were characterized as likely discontinuous and consisting of residues found in the sequence ranges 158-172 YLPYKVLPVGDEVVG (SEQ ID NO: 108) and 206-215 KLKTLNVNKI (SEQ ID NO: 109) of human CD73, numbered according to SEQ ID NO: 105. If one considers a model that requires locking of CD73 into the inactive conformation for inhibition, then based on the experimental observations from HDx MS and SEC, one would predict that 373.A, 373.B, 350.A2, and 350.B are able to inhibit soluble and membrane bound forms of CD73.

### Example 3: Up-Regulation of CD73 in Stromal Cells Upon TGFβ Blockade In Vivo

CD73 expression in cancer has been reported not only in cancer cells but also in the stromal elements that make up the tumor microenvironment. Additionally, several stromal factors, including TGFβ, have been suggested to provide potential amplification mechanisms for adenosine generation in tissue microenvironment. In this example, a study was conducted to determine whether TGFβ blockade in vivo may prompt changes in expression of CD73 in tumor stromal cells.

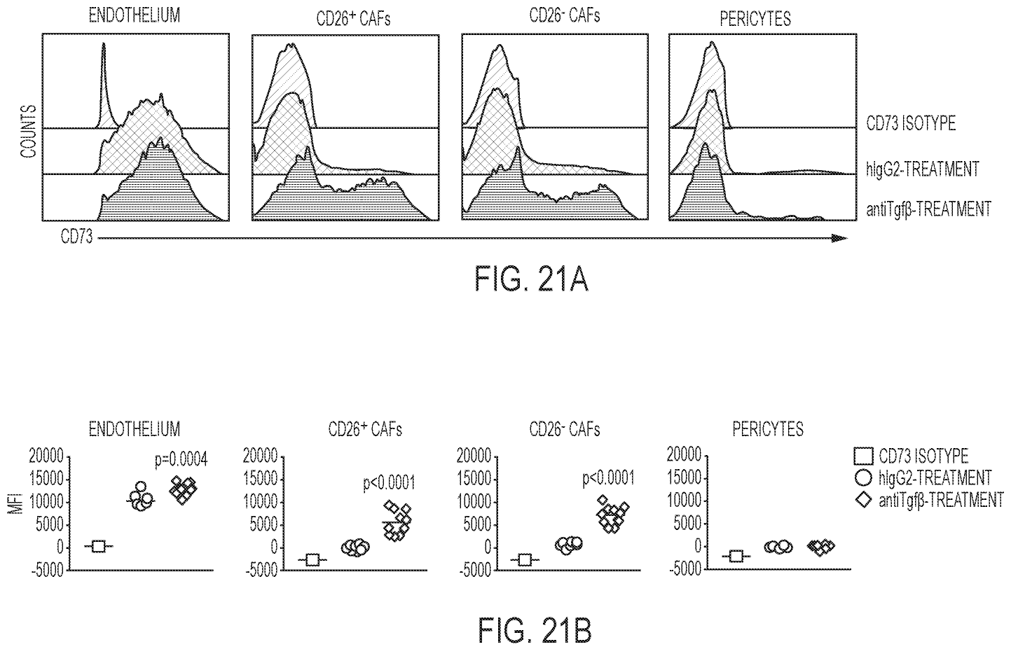

To this end, a humanized IgG2 monoclonal antibody that binds to human and murine TGFβ1 and TGFβ2 with high affinity and to TGFβ with lower affinity was utilized. This antibody has been demonstrated to possess neutralizing activity against TGFβ isoforms in vitro and in vivo. Immunocompetent, BALB/c, female mice (6-8 weeks of age) were implanted in the right flank with 4T1 murine triple-negative breast cancer cell line at 10×10⁴ cells/mouse/100 μl. Ten mice per group were treated intraperitoneally, every other day with either a pan TGFβ-blocking antibody or isotype control (hIgG2), both at a final concentration of 10 mg/kg of body weight. Tumors were allowed to grow for 14 days, after which animals were euthanized and tumors collected and subjected to flow cytometric analysis. Briefly, tumors were excised from mice and digested using a combination of physical and enzymatic dissociation (DNasel, CollagenaseP, Dispase). Single cell suspensions were Fc-blocked for 30 minutes to prevent nonspecific antibody binding, and stained with a panel of antibodies that bind to specific cell surface markers including CD45 (to exclude hematopoietic cells), CD31 (endothelial cells), CD90 (pan mesenchyrnal marker), CD26 and podoplanin, to allow identification of stromal cell populations. CD73 expression was assessed using an eFluor450-conjugated anti-CD73 antibody (eBioscience #484)731-82), as compared to isotype-controlled stained samples (eFluor 450-conjugated Rat IgG1, eBioscience #48-4301-82). After staining for 40 minutes at 4° C., samples were washed with FACS buffer (PBS, 5 mM EDTA, 2% Fetal Bovine Serum), fixed with Fix/Perm solution (eBioscience #00-5523) following the manufacturer's instructions, and analyzed on a BD LSRFortessa™ flow cytometer. CD73 was highly expressed in endothelial cells in isotype control-treated mice, while minimal expression was detected in mesenchymal stromal cells (CD26+ and CD26-CAFs, and pericytes) (FIGS. 21A and 21B). Strikingly, treatment of mice with TGFβ-neutralizing antibodies prompted a significant up-regulation of CD73 in cancer associated fibroblasts (CAFs), independently of CD26 expression (FIGS. 21A and 21B). Up-regulation of CD73 was also noticeable in endothelial cells, although to a lesser extent, while no up-regulation was observed in pericytes (FIGS. 21A and 21B). Altogether, these data indicate that neutralizing TGFβ in the tumor microenvironment elicits expression of CD73 in stromal cells, suggesting that up-regulation of CD73 and adenosine production by tumor fibroblasts may provide an alternative mechanism co-opted by tumors to maintain immune quiescent upon TGFβ blockade.

### Example 4: Impact of Anti-CD73 Antibodies on B Cells

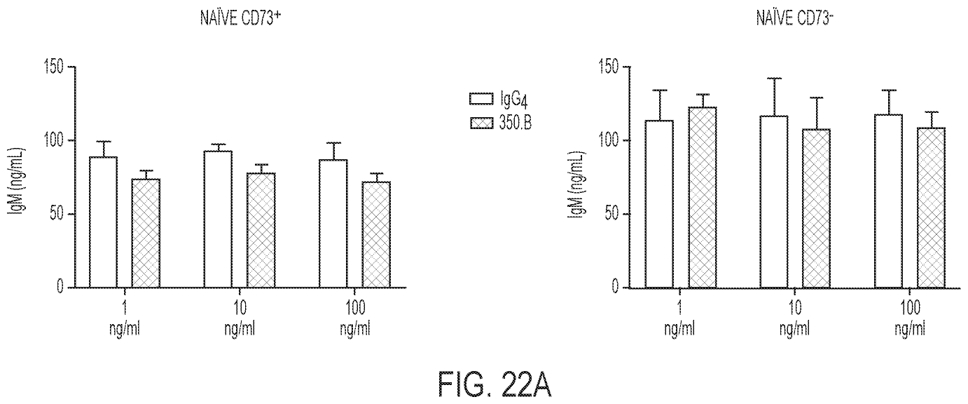

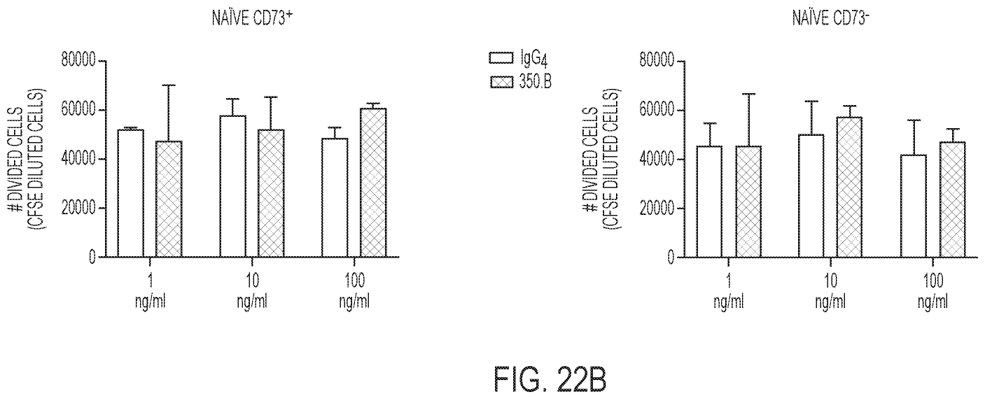

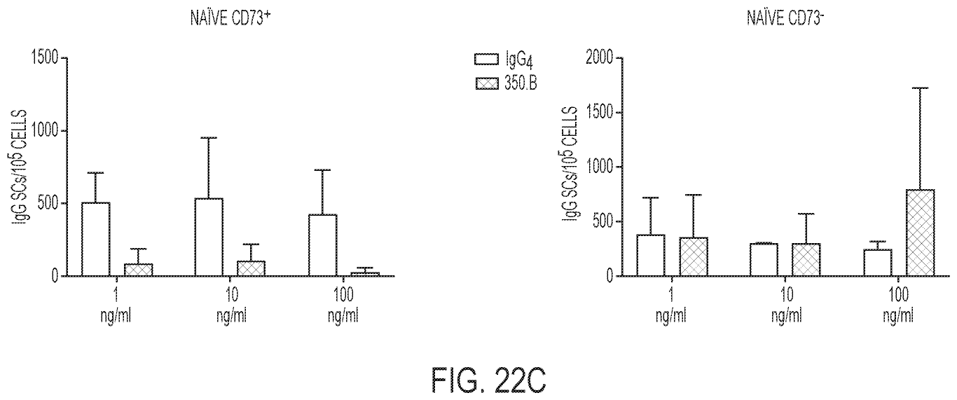

To assess whether blockade of CD73-dependent adenosine generation could specifically inhibit class switch recombination (CSR) without impacting B cell response efficiency, the anti-CD73 antibody 350.B was tested. Naive B (CD19+CD27-IgM+IgD+) cells were purified from peripheral blood of two healthy donors, based on CD73 expression, and stimulated with a TLR9 agonist, an anti-CD40 antibody, and cytokines (IL-2 and IL-21) in the presence of the antibody 350.B. Three different concentrations of the antibody were tested ˜1 ng/ml, 10 ng/ml and 100 ng/ml—along with an IgG4 isotype control. IgM secretion was measured at day 7 in culture supernatants, and no differences were observed compared to the isotype control (FIG. 22A). Notably, inhibition of CD73-enzymatic activity with 350.B treatment resulted in decreased differentiation of IgG secreting cells, as measured by an ELISPOT assay to detect IgG secreting plasma cells (Millipore MSIPS4510 plates, coated with 10 μg/ml goat anti-human IgG, Southern Biotechnologies), with a strong effect at the concentration of 100 ng/ml (FIG. 22C). The number of viable cells was also evaluated, and no differences were observed (FIG. 22B), ruling out the link between low survival rate and poor CSR efficiency observed in the naive CD73+ B cells after treatment with 350.B. As a control, naive CD73− cells were equally stimulated, and no differences were observed (FIGS. 22A-C). Finally, the evaluation of CSR in the isotype control conditions confirmed increased number of IgG ISCs in the naive CD73+ compartment (FIG. 22C). Taken together, these results demonstrated that blockade of CD73 enzymatic activity has an impact on class switch recombination without altering the IgM response.

### Example 5: Anti-CD73 and Anti-PD-1 Co-Blockade in Syngeneic Mouse Tumor Models

To determine the role of co-blockade of the CD73 and PD-1 pathways in tumor-bearing mice, anti-PD-1 and anti-CD73 (350.B) antibodies were administered to immuno-competent mice inoculated with a syngeneic colon carcinoma cell line, CT-26. BALB/c mice aged 6-10 weeks each were inoculated on the flank with 1×10⁵ CT-26 tumor cells. On day 2 after inoculation, mice (n=10/group) were treated intraperitoneally with either isotype control antibody, 350.B 600 μg for 1 dose followed by 400 μg for 4 doses; anti-PD-1 300 μg for 5 doses (low dose); anti-PD-1 600 μg for 5 doses (high dose); a combination of high-dose anti-PD-1+350.B; or a combination of low-dose anti-PD-1+350.B. Tumor dimensions and body weights were recorded three times in a week with a gap of 2 to 3 days in between measurements, and each animal was euthanized when the tumor size reached >2000 mm³, if the mouse was moribund, or if the tumor was ulcerated. Percent tumor growth inhibition through day 25 was calculated (because the control group was euthanized on day 26).

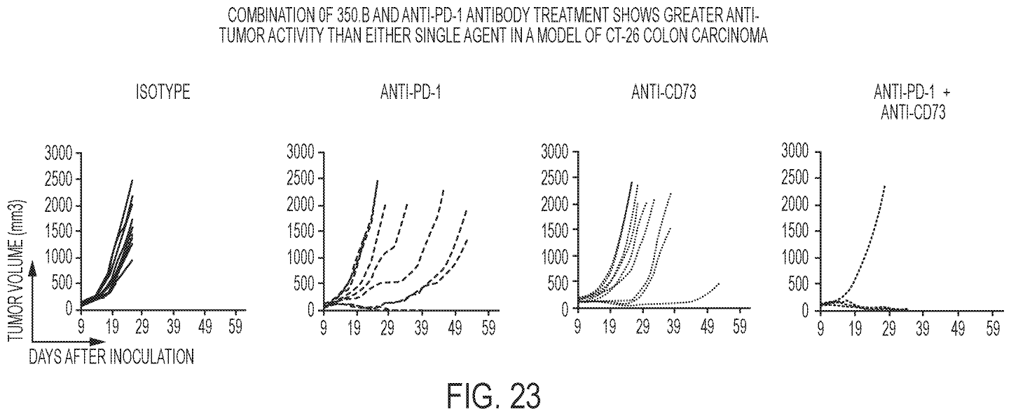

Treatment with 350.B or low-dose anti-PD-1 alone (300 μg) showed partial tumor growth inhibition (30.96% and 52.04%, respectively) on day 25. In contrast, low-dose anti-PD-1+350.B in combination resulted in 88.41% tumor growth inhibition (FIG. 23). Tumor control with a high dose of anti-PD-1 alone was sufficient such that additional benefit with 350.B was not observed. No notable loss in body weights was observed in any of the groups, and there were no significant clinical observations for the animals on study. These data demonstrate that combination treatment of sub-optimal doses of anti-CD73 and anti-PD-1 antibodies can lead to enhanced tumor growth inhibition in the CT-26 syngeneic tumor model.

### Example 6: Pharmacokinetics and Toxicology Study of the Anti-CD73 Antibody 373.a in Cynomolgus Monkeys

As a single agent, the anti-CD73 antibody 373.A was well tolerated when intravenously administered once weekly over four weeks to cynomolgus monkeys at doses as high as 100 mg/kg. There were no effects on cardiac function as assessed by electrocardiogram nor were there any toxicologically meaningful effects on clinical pathology, immunophenotyping or histopathological findings. Therefore, the no observed adverse effect level (NOAEL) for 373.A in cynomolgus monkeys is 100 mg/kg, whereas the highest non-severely toxic dose (HNSTD) is >100 mg/kg.

In a Non GLP single dose toxicology study, cynomolgous monkeys were administered single IV doses of control, 3, 30 or 100 mg/kg of the antibody 373.A. After IV injection, 373.A declined rapidly in the 3 mg/kg group likely due to target-mediated drug disposition. In the 30 mg/kg and 100 mg/kg group decline was generally in a multi-exponential manner No sex differences in 373.A Cmax and AUC₀₋₁₆₈ₕᵣ values were observed. Exposure, assessed by 373.A mean Cmax and AUC₀₋₁₆₈ₕᵣ values, increased with the increase in dose levels from 3 to 100 mg/kg. The increases in 373.A mean Cmax and AUC₀₋₁₆₈ₕᵣ values were generally dose proportional, with the exception of the AUC₀₋₁₆₈ₕᵣ between dose levels of 3 and 30 mg/kg. In the 100 mg/kg dose cohort, mean Cmax and AUC₀₋₁₆₈ₕᵣ were 2540 μg/mL and 173,000 μg·hr/mL, respectively. Anti-drug antibody (ADA) incidence in this study did not affect the PK.

In a GLP toxicology study, monkeys were administered four weekly IV doses of control, 10, 30 or 100 mg/kg of 373.A. Sex differences in 373.A Cmax and AUC₀₋₁₆₈ₕᵣ were minimal (between 0.7 to 1.2 fold). Exposure, as assessed by mean Cmax and AUC₀₋₁₆₈ₕᵣ, increased proportionally from 10 to 100 mg/kg. After IV bolus injection, 373.A PK declined in a bi-exponential manner, with a mean elimination phase t_{1/2} value of 266 hours following the recovery phase on Day 22 in the 100 mg/kg group. In the 100 mg/kg dose cohort, mean Cmax and AUC₀₋₁₆₈ hr after the day 22 dose, were 5030 μg/mL and 365,000 μg·hr/mL, respectively. ADA incidence decreased exposure in one animal in the 30 mg/kg group.

In a separate study, the cytokine release was assessed in an in-vitro soluble assay format following a 24-hour stimulation of human whole blood with 373.A, at 50, 100, 500 and 1000 μg/mL.

Blood samples were collected from ten healthy human donors. For each sample, negative (whole blood and whole blood with medium) and positive (anti-human CD3) controls were included. Cytokine levels were measured using a Luminex multiplex method for IL-1β, IL-2, IL-6, IL-8, IL-10, IFNγ, and TNFα. Results obtained with the Buffer control were similar to the results obtained with the negative control (blood only) and no trend towards a decrease or an increase in cytokine release was noted with the diluent (0.5% Dextrose). For all donors, there was cytokine release induction with the anti-CD3 positive controls for all cytokines, indicating that the stimulation was appropriate and cytokine release could be induced in all samples. Cytokine levels obtained following stimulation with 373.A were similar to the levels measured in the negative controls. In conclusion, in-vitro stimulation of human whole blood with 373.A did not induce cytokine release at concentrations as high as 1000 μg/mL.

### Example 7: A Phase I/Ib Study of the Anti-CD73 Antibody 373.A as a Single Agent and in Combination with BAP049-Clone-E and/or PBF509 in Patients with Advanced Malignancies

During the last decade, immunotherapies that target different immune checkpoints (e.g., PD-1, PD-L1 and CTLA-4) have shown efficacy in a numbers of cancer indications. However, while some patients achieve objective and long lasting responses to checkpoint blockade, the majority of patients show modest or no clinical benefit, indicating that tumors use alternative immunosuppressive mechanisms to achieve immune escape (Allard et al., Clin Cancer Res. 2013; 19(20):5626-35; Vesely et al., Annu Rev Immunol 2011; 29:235-271). Thus, concomitant blockade of multiple immune suppressive pathways may be required to induce clinically meaningful responses in a larger number of patients.

Over the past years, adenosine generation and signaling have emerged as potential therapeutic targets in cancer treatment. Adenosine creates an immunosuppressive tumor microenvironment by reducing the cytotoxic anti-tumor immune response, enhancing the proliferation and polarization of immune suppressive cells, and by increasing neovascularization (Young et al., Cancer Discovery 2014; 4(8):879-88). Preclinical data demonstrate that CD73 blockade can significantly delay primary tumor growth and inhibit the development of lung metastases in an immune-competent syngeneic mouse model (Stagg et al 2010). Similar results were observed in a study where genetic deletion of A2aR in the host resulted in rejection of the established immunogenic tumors in A2aR deficient mice with no rejection seen in control wild type mice (Ohta et al., PNAS 2006; 103(35):13132-37).

A phase I/Ib, open-label, multi-center study has been designed to evaluate the safety, tolerability, preliminary anti-tumor activity, pharmacokinetics (PK) and pharmacodynamics (PD) of the anti-CD73 antibody 373.A as a single agent and in combination with the A2a_{A}R antagonist PBF509 and/or the anti-PD-1 antibody BAP049-Clone-E in patients with advanced malignancies. The primary objectives are to characterize the safety and tolerability, and to determine the recommended dose (RD) for 373.A as a single agent and in combination with PBF509 and/or BAP049-Clone-E. The secondary objectives are to assess the preliminary anti-tumor activity and PK of 373.A as a single agent and in combination with PBF509 and/or BAP049-Clone-E, assess the immunogenicity of 373.A and BAP049-Clone-E, and to characterize changes in the immune infiltrate in tumors following treatment, e.g., change from baseline in tumor infiltrating lymphocytes (TILs), tumor associated macrophages (TAMs), CD8+ T-cells, and PDL-1 expression.

BAP049-Clone-E is a high-affinity, ligand-blocking, humanized anti-programmed death-1 (PD-1) IgG4 antibody that blocks the binding of PD-L1 and PD-L2 to PD-1. BAP049-Clone-E is being tested in a phase I/II study in advanced malignancies. PBF509, a new, non-xanthine-based compound, is a potent oral adenosine A2a_{A}R antagonist. The sequence of BAP049-Clone-E is disclosed in Table 5.

Two ongoing Phase Mb and Phase II studies evaluate PBF509 as a single agent and/or in combination with BAP049-Clone-E in patients with advanced non-small cell lung cancer (NSCLC) and solid tumors and non-Hodgkin lymphoma, respectively.

This I/Ib study will initially enroll adult patients with advanced malignancies that have progressed or are intolerant to standard therapy in indications where moderate to high CD73 expression has been associated with poorer outcome, indicating adenosine-mediated immune escape (Wu et al., Journal of Surgical Oncology 2012, 106(2): 130-137; Gaudreau et al., Oncoimmunology; 2016, 5(5): e1127496; Inoue et al., Oncotarget.; 2017, 8(5):8738-8751). These indications include non-small cell lung cancer (NSCLC), triple negative breast cancer (TNBC), pancreatic cancer (PDAC), renal cell carcinoma (RCC), ovarian cancer, and micro-satellite stable (MSS) colorectal cancer, though additional indications may be enrolled based on emerging clinical data (e.g. efficacy data or proven pathway activation).

The study consists of two parts: (1) a dose escalation part for single agent 373.A, doublet combinations 373.A/PBF509 and 373.A/BAP049-Clone-E, or a triplet combination 373.A/PBF509/BAP049-Clone-E, which leads to declaration of recommended doses (RDs) for each treatment, and (2) a dose expansion part where patients will be treated at the RDs for single agent, doublet combinations, and triplet combination. The escalation part will enroll patients with advanced NSCLC, TNBC, PDAC, RCC, ovarian cancer, and colorectal cancer (MSS); and there is no restriction on the number of prior treatments. The expansion part will enroll patients with advanced malignancies having received up to 3 lines of prior treatment.

In the expansion part, patients in each indication will be equally randomized to the combination treatment arms. Randomization will be performed per indication, and further stratified within certain indications by prior PD-1/PD-L1 treatment (naive or resistant).

### Dose and Regimen Selection

### 373.A Monotherapy

The starting dose of 60 mg flat dose 373.A, administered intravenously every 2 weeks (Q2W), was selected based upon preclinical safety, tolerability, and PK data observed in cynomolgus monkey as well as published case histories of CD73 deficient patients.

The 60 mg dose is considered to be a minimally pharmacological active dose (mPAD) as it is predicted to provide (1) approximately 20 hrs of >90% CD8+ T-cell CD73 occupancy, (2) approximately 22 hrs of >90% adenosine inhibition, (3) approximately 17 hrs of >90% imputed overall CD73 occupancy.

Based on modeling of TK data from the cynomolgus monkey Toxicology studies, ex-vivo CD8+ T cell CD73 occupancy data and in-vitro data on inhibition of adenosine formation, a dose >1200 mg Q2W is predicted to achieve >90% target occupancy on CD8+ T cells throughout the dosing interval and a dose ≥600 mg Q2W is predicted to achieve >90% inhibition of adenosine production.

The dose of 373.A will be escalated in sequential cohorts, guided by a Bayesian Logistic Regression model (BLRM) coupled with overdose control (EWOC) criterion, until a maximum tolerated dose (MTD) or recommended dose (RD) for expansion is identified. Preclinical data and modeling suggest that there may be a high antigen sink and that high doses (e.g. ≥1200 mg Q2W) may be needed to achieve continuous target occupancy throughout the dose interval. Dose escalation will primarily be performed with a Q2W regimen. However, if this regimen shows rapid 373.A elimination and lack of target saturation within the dosing interval, a more frequent QW regimen may be tested. If on the other hand, a Q4W regimen is predicted to have no rapid elimination within the dosing interval, Q4W regimen may be explored instead.

### 373.A/PBF509 Combination

The maximum starting doses for the 373.A/PBF509 doublet combination will be 200 mg Q2W 373.A and 80 mg BID PBF509.

200 mg Q2W 373.A is a low dose of 373.A that is predicted to achieve ˜2.3 days of >90% target occupancy on CD8+ T cells. The 200 mg Q2W 373.A dose is 16% of the 1200 mg Q2W dose that is anticipated to achieve >90% CD8+ T cell target occupancy throughout the dosing interval.

PBF509 has been tested as a single-agent up to 640 mg BID with no safety issues in advanced/metastatic NSCLC (only 1 DLT out of 5 evaluable patients at the 640 mg BID dose). In the same study, PBF509 has shown single-agent activity in the range of 80 mg BID to 480 mg BID, with 2 partial responses (PRs) and 6 with stable disease (SD), in a total of 18 dosed patients.

A dose-escalation approach of 373.A and PBF509 will be undertaken in order to determine the appropriate dose of each drug in combination, guided by Bayesian Logistic Regression modeling (BLRM) coupled with overdose control (EWOC) principle criteria.

### 373.A/BAP049-Clone-E Combination

The maximum starting dose for the 373.A/BAP049-Clone-E doublet combination will be 200 mg Q2W 373.A and 400 mg Q4W BAP049-Clone-E.

The rationale for 200 mg Q2W 373.A has been described above. The 200 mg Q2W 373.A will be combined with the RD for BAP049-Clone-E which is 400 mg Q4W, which has been shown to be safe and efficacious.

373.A dose level will be escalated sequentially with a fixed dose of BAP049-Clone-E, guided by Bayesian Logistic Regression modeling (BLRM) coupled with overdose control (EWOC) principle criteria.

### 373.A/BAP049-Clone-E/PBF509 Combination

The maximum starting dose for the 373.A/BAP049-Clone-E/PBF509 triplet combination will be 200 mg Q2W 373.A, 400 mg Q4W BAP049-Clone-E and 80 mg BID PBF509.

PBF509 has been safely administered up to 160 mg BID in combination with 400 mg Q4W BAP049-Clone-E (only 1 DLT out of 6 evaluable patients), with combination activity on efficacy: 2 PRs and 6 SDs, out of a total of 10 patients. The 160 mg BID PBF509 and 400 mg Q4W BAP049-Clone-E dose is currently being used in the Ph2 study.

A dose-escalation approach for 373.A/BAP049-Clone-E/PBF509 with a fixed dose of BAP049-Clone-E will be undertaken in order to determine the appropriate dose of 373.A and PBF509 in the triplet combination, guided by a Bayesian Logistic Regression model (BLRM) coupled with overdose control (EWOC) criterion.

The antibody 373.A (100 mg powder for solution for infusion) will be administered intravenously as a 1 hr infusion (up to 2 hours if clinically indicated). BAP049-Clone-E (100 mg powder for solution for infusion) will be administered intravenously as a 30 minute infusion (up to 2 hours, if clinically indicated). When given in combination, 373.A and BAP049-Clone-E are to be administered on the same day using separate infusion materials (bag, lines, filters) for each infusion. The same access site may be used for both infusions. 373.A will be infused first followed by a 30 minute break before infusing BAP049-Clone-E. PBF509 (40 mg and/or 80 mg and/or 160 mg capsule for oral use) will be taken orally twice daily (BID) continuously. On the visits where 373.A and/or BAP049-Clone-E will be administered, the dose of PBF509 will be taken first followed by the 373.A infusion. A break between PBF509 administration and the 373.A infusion is not required. The 373.A infusion should be started as soon as possible and not more than 60 minutes after PBF509 is given.

Tables 22-25 describe the starting dose and the dose levels that may be evaluated during this trial.

Patients treated with 373.A single agent or 373.A in combination with BAP049-Clone-E and/or PBF509 will begin study treatment on Cycle 1 Day 1. Each cycle will consist of 28 days. 373.A Q2W will be administered on Day 1 and 15 of a cycle. 373.A QW will be administered on Day 1, 8, 15, and 22 of a cycle. 373.A Q4W will be administered on Day 1 of a cycle. BAP049-Clone-E Q4W will be administered on Day 1 of a cycle. PBF509 BID will be taken every day of a cycle.

TABLE 22
Provisional dose levels for 373.A

| | Proposed dosing | Increment from |
|---|---|---|
| Dose level | regimen* | previous dose |
| −2** | 6 mg Q2W | −233% |
| −1** | 20 mg Q2W | −200% |
| 1 | 60 mg Q2W | (starting dose) |
| 2 | 200 mg Q2W | 233% |
| 3 | 600 mg Q2W | 200% |
| 4 | 1200 mg Q2W | 100% |
| 5 | 2400 mg Q2W | 100% |
| 6 | 3600 mg Q2W | 50% |

*It is possible for additional and/or intermediate dose levels to be added during the course of the study Cohorts may be added at any dose level below the MTD in order to better understand safety, PK or PD.
**Dose level −1 and −2 represent treatment doses for patients requiring a dose reduction from the starting dose level. No dose reduction below dose level −2 is permitted for this study.

TABLE 23
Provisional dose levels for 373.A in combination with PBF509

| | Proposed | Increment from | Proposed | Increment from |
|---|---|---|---|---|
| Dose | dose | previous dose | dose | previous dose |
| level | 373.A* | 373.A | PBF509* | PBF509 |
| −2** | 20 mg Q2W | −200% | 40 mg BID | 0% |
| −1** | 60 mg Q2W | −233% | 40 mg BID | −100% |
| 1 | 200 mg Q2W | (starting dose) | 80 mg BID | (starting dose) |
| 2 | 200 mg Q2W | 0% | 160 mg BID | 100% |
| 3 | 600 mg Q2W | 200% | 160 mg BID | 0% |
| 4 | 1200 mg Q2W | 100% | 160 mg BID | 0% |
| 5 | 2400 mg Q2W | 100% | 160 mg BID | 0% |
| 6 | 2400 mg Q2W | 100% | 320 mg BID | 100% |
| 7 | 2400 mg Q2W | | 480 mg BID | |
| 8 | 2400 mg Q2W | | 620 mg BID | |
| 9 | 3600 mg Q2W | | 320 mg BID | |
| 10 | 3600 mg Q2W | | 480 mg BID | |
| 11 | 3600 mg Q2W | | 620 mg BID | |

*It is possible for additional and/or intermediate dose levels to be added during the course of the study Cohorts may be added at any dose level below the MTD in order to better understand safety, PK or PD.
**Dose level −1 and −2 represent treatment doses for patients requiring a dose reduction from the starting dose level. No dose reduction below dose level −2 is permitted for this study.

TABLE 24
Provisional dose levels for 373.A
in combination with BAP049-Clone-E

| | | Increment | Proposed | Increment |
|---|---|---|---|---|
| | Proposed | from | dose | from previous |
| Dose | dose | previous | BAP049- | dose BAP049- |
| level | 373.A* | dose 373.A | Clone-E | Clone-E |
| −2** | 20 mg Q2W | −200% | 400 mg Q4w | 0% |
| −1** | 60 mg Q2W | −233% | 400 mg Q4w | 0% |
| 1 | 200 mg Q2W | (starting | 400 mg Q4w | (starting |
| | | dose) | | dose) |
| 2 | 600 mg Q2W | 200% | 400 mg Q4w | 0% |
| 3 | 1200 mg Q2W | 100% | 400 mg Q4w | 0% |
| 4 | 2400 mg Q2W | 100% | 400 mg Q4w | 0% |
| 5 | 3600 mg Q2W | 50% | 400 mg Q4W | 0% |

*It is possible for additional and/or intermediate dose levels to be added during the course of the study Cohorts may be added at any dose level below the MTD in order to better understand safety, PK or PD.
**Dose level −1 and −2 represent treatment doses for patients requiring a dose reduction from the starting dose level. No dose reduction below dose level −2 is permitted for this study.

TABLE 25
Provisional dose levels for PBF509 in combination with 373.A and BAP049-Clone-E

| | | | | Increment | | |
|---|---|---|---|---|---|---|
| | | Increment | Proposed | from previous | | Increment |
| Dose | Proposed | from previous | dose BAP049- | dose BAP049- | Proposed | from previous |
| level | dose 373.A* | dose 373.A | Clone-E | Clone-E | dose PBF509* | dose PBF509 |
| −2** | 20 mg Q2W | −200% | 400 mg Q4W | 0% | 40 mg BID | 0% |
| −1** | 60 mg Q2W | −233% | 400 mg Q4W | 0% | 40 mg BID | −100% |
| 1 | 200 mg Q2W | (starting | 400 mg Q4W | (starting | 80 mg BID | (starting |
| | | dose) | | dose) | | dose) |
| 2 | 200 mg Q2W | 0% | 400 mg Q4W | 0% | 160 mg BID | 100% |
| 3 | 600 mg Q2W | 200% | 400 mg Q4W | 0% | 160 mg BID | 0% |
| 4 | 1200 mg Q2W | 100% | 400 mg Q4W | 0% | 160 mg BID | 0% |
| 5 | 2400 mg Q2W | 100% | 400 mg Q4W | 0% | 160 mg BID | 0% |
| 6 | 2400 mg Q2W | | 400 mg Q4W | | 320 mg BID | |
| 7 | 2400 mg Q2W | | 400 mg Q4W | | 480 mg BID | |
| 8 | 2400 mg Q2W | | 400 mg Q4W | | 620 mg BID | |
| 9 | 3600 mg Q2W | | 400 mg Q4W | | 320 mg BID | |
| 10 | 3600 mg Q2W | | 400 mg Q4W | | 480 mg BID | |
| 11 | 3600 mg Q2W | | 400 mg Q4W | | 620 mg BID | |

*It is possible for additional and/or intermediate dose levels to be added during the course of the study Cohorts may be added at any dose level below the MTD in order to better understand safety, PK or PD.
**Dose level −1 and −2 represent treatment doses for patients requiring a dose reduction from the starting dose level. No dose reduction below dose level −2 is permitted for this study.

### Example 8: Detection of Soluble CD73 (sCD73)

Detection of sCD73 can be undertaken in patient sera or plasma to determine circulating levels of sCD73 protein both pre- and post-treatment with a therapeutic anti-CD73 antibody molecule.

A sandwich ELISA-type assay has been developed using the MesoScale Discovery platform to detect sCD73 in the presence of treatment of patients with a therapeutic anti-CD73 antibody molecule. Plates are coated with capture anti-CD73 antibody 913. The sequences of antibody 913 are disclosed in Table 1. In assay optimization, recombinant CD73 protein (R&D Systems 5795-EN-010), in the presence or absence of a therapeutic anti-CD73 antibody molecule, is added and then detected with a sulfo-tagged anti-CD73 monoclonal antibody, 7G2 (Abcam ab54217). Plates are washed, a surfactant read buffer is added (MSD R32AD-1) and electrochemiluminescence signal is measured by MA600 Reader (MesoScale Discovery).

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. An antibody molecule that binds to human CD73, comprising:
a light chain variable region (VL) comprising a light chain complementarity determining region 1 (VLCDR1), a VLCDR2, and a VLCDR3 comprising the amino acid sequences of SEQ ID NOs: 344, 345, and 346, respectively; and
a heavy chain variable region (VH) comprising a heavy chain complementarity determining region 1 (VHCDR1), a VHCDR2, and a VHCDR3 comprising the amino acid sequences of:
(i) SEQ ID NOs: 213, 211, and 212, respectively;
(ii) SEQ ID NOs: 335, 288, and 334, respectively; or
(iii) SEQ ID NOs: 273, 271, and 272, respectively.

2. The antibody molecule of claim 1, wherein the VHCDR1, VHCDR2, and VHCDR3 comprise the amino acid sequences of SEQ ID NOs: 213, 211, and 212, respectively.

3. The antibody molecule of claim 1 or 2, wherein the VLCDR1, VLCDR2, and VLCDR3 comprise the amino acid sequences of:
(i) SEQ ID NOs: 221, 222, and 223, respectively;
(ii) SEQ ID NOs: 221, 222, and 341, respectively; or
(iii) SEQ ID NOs: 281, 15, and 282, respectively.

4. The antibody molecule of any one of claims 1-3, wherein the VHCDR1, VHCDR2, VHCDR3, VLCDR1, VLCDR2, and VLCDR3 comprise the amino acid sequences of:
(i) SEQ ID NOs: 213, 211, 212, 221, 222, and 223, respectively;
(ii) SEQ ID NOs: 335, 288, 334, 221, 222, and 341, respectively; or
(iii) SEQ ID NOs: 273, 271, 272, 281, 15, and 282, respectively.

5. The antibody molecule of any one of claims 1-4, wherein the VHCDR1, VHCDR2, VHCDR3, VLCDR1, VLCDR2, and VLCDR3 comprise the amino acid sequences of SEQ ID NOs: 213, 211, 212, 221, 222, and 223, respectively.

6. The antibody molecule of any one of claims 1-5, comprising a VH comprising the amino acid sequence of SEQ ID NO: 220, 340, or 280, or an amino acid sequence having at least about 85%, 90%, 95%, or 99% sequence identity thereof.

7. The antibody molecule of any one of claims 1-6, comprising a VL comprising the amino acid sequence of SEQ ID NO: 227, 343, or 286, or an amino acid sequence having at least about 85%, 90%, 95%, or 99% sequence identity thereof.

8. The antibody molecule of any one of claims 1-7, comprising a VH and a VL comprising the amino acid sequences of:
(i) SEQ ID NOs: 220 and 227, respectively;
(ii) SEQ ID NOs: 340 and 343, respectively; or
(iii) SEQ ID NOs: 280 and 286, respectively.

9. The antibody molecule of any one of claims 1-8, comprising a VH and a VL comprising the amino acid sequences of SEQ ID NOs: 220 and 227, respectively.

10. The antibody molecule of any one of claims 1-9, comprising a VH having an amino acid sequence derived from a human VH4-34, VH1-69, or VH4-39 germline sequence.

11. The antibody molecule of any one of claims 1-10, comprising a VL having an amino acid sequence derived from a human VK1-39 germline sequence.

12. The antibody molecule of any one of claims 1-11, comprising a VH having an amino acid sequence derived from a human VH4-34 germline sequence and a VL having an amino acid sequence derived from a human VK1-39 germline sequence.

13. An antibody molecule that binds to human CD73, comprising:
a VH comprising a VHCDR1, a VHCDR2, and a VHCDR3, wherein:
(i) the VHCDR1 comprises the amino acid sequence of SEQ ID NO: 347;
(ii) the VHCDR2 comprises the amino acid sequence of SEQ ID NO: 288; and
(iii) the VHCDR3 comprises the amino acid sequence of SEQ ID NO: 334 or 289; and
a VL comprising a VLCDR1, a VLCDR2, and a VLCDR3 comprising the amino acid sequences of:
(i) SEQ ID NOs: 221, 222, and 341, respectively; or
(ii) SEQ ID NOs: 298, 49, and 299, respectively.

14. The antibody molecule of claim 13, wherein the VHCDR1, VHCDR2, and VHCDR3 comprise the amino acid sequences of:
(i) SEQ ID NOs: 335, 288, and 334, respectively; or
(ii) SEQ ID NOs: 290, 288, and 289, respectively.

15. The antibody molecule of claim 13 or 14 wherein the VHCDR1, VHCDR2, VHCDR3, VLCDR1, VLCDR2, and VLCDR3 comprise the amino acid sequences of:
(i) SEQ ID NOs: 335, 288, 334, 221, 222, and 341, respectively; or
(ii) SEQ ID NOs: 290, 288, 289, 298, 49, and 299, respectively.

16. The antibody molecule of any one of claims 13-15, comprising a VH comprising the amino acid sequence of SEQ ID NO: 340 or 297, or an amino acid sequence having at least about 85%, 90%, 95%, or 99% sequence identity thereof.

17. The antibody molecule of any one of claims 13-16, comprising a VL comprising the amino acid sequence of SEQ ID NO: 343 or 303, or an amino acid sequence having at least about 85%, 90%, 95%, or 99% sequence identity thereof.

18. The antibody molecule of any one of claims 13-17, comprising a VH and a VL comprising the amino acid sequences of:
(i) SEQ ID NOs: 340 and 343, respectively; or
(ii) SEQ ID NOs: 297 and 303, respectively.

19. The antibody molecule of any one of claims 13-18, comprising a VH having an amino acid sequence derived from a human VH1-69 germline sequence.

20. The antibody molecule of any one of claims 13-19, comprising a VL having an amino acid sequence derived from a human VK1-39 or VK3-15 germline sequence.

21. The antibody molecule of any one of claims 1-20, which is a human antibody, a full length antibody, a bispecific antibody, Fab, F(ab′)2, Fv, or a single chain Fv fragment (scFv).

22. The antibody molecule of any one of claims 1-21, comprising a heavy chain constant region selected from IgG1, IgG2, IgG3, and IgG4, and a light chain constant region chosen from the light chain constant regions of kappa or lambda.

23. The antibody molecule of any one of claims 1-22, comprising a heavy chain constant region comprising an amino acid sequence selected from the group consisting of SEQ ID NOs: 92-103, 119, and 120, and/or a light chain constant region comprising the amino acid sequence of SEQ ID NO: 104.

24. An antibody molecule that competes for binding to human CD73 with the antibody molecule of any one of claims 1-23.

25. An antibody molecule that binds to the same epitope as, substantially the same epitope as, an epitope that overlaps with, or an epitope that substantially overlaps with, the epitope of the antibody molecule of any one of claims 1-24.

26. A pharmaceutical composition comprising the antibody molecule of any one of claims 1-25 and a pharmaceutically acceptable carrier, excipient or stabilizer.

27. A nucleic acid encoding the antibody heavy or light chain variable region of the antibody molecule of any one of claims 1-25.

28. An expression vector comprising the nucleic acid of claim 27.

29. A host cell comprising the nucleic acid of claim 27 or the expression vector of claim 28.

30. A method of producing an antibody molecule, the method comprising culturing the host cell of claim 29 under conditions suitable for gene expression.

31. A method of detecting CD73 in a biological sample or in a subject, comprising (i) contacting the sample or the subject (and optionally, a reference sample or subject) with the antibody molecule of any one of claims 1-25 under conditions that allow interaction of the antibody molecule and CD73 to occur, and (ii) detecting formation of a complex between the antibody molecule and CD73.

32. A method of detecting soluble CD73 in a biological sample (e.g., serum or plasma) from a subject, comprising (i) contacting the biological sample (e.g., serum or plasma) with the antibody molecule of any one of claims 1-25 under conditions that allow interaction of the antibody molecule and soluble CD73 to occur, and (ii) detecting formation of a complex between the antibody molecule and soluble CD73.

33. The method of claim 32, wherein the subject has received or is going to receive administration of a therapeutic agent, e.g., one, two or all of a therapeutic anti-CD73 antibody molecule, a PD-1 inhibitor and an adenosine A2AR antagonist.

34. A method of evaluating responsive of a subject to a therapeutic agent, wherein the subject has received or is going to receive administration of the therapeutic agent (e.g., one, two or all of a therapeutic anti-CD73 antibody molecule, a PD-1 inhibitor and an adenosine A2AR antagonist), comprising acquiring the level of soluble CD73 in the subject, e.g., at one or more time points prior to, during, or after the administration of the therapeutic agent, wherein acquiring the level of soluble CD73 comprises, e.g., contacting the subject, e.g., a biological sample (e.g., serum or plasma) from the subject, with the antibody molecule of any one of claims 1-25 under conditions that allow interaction of the antibody molecule and soluble CD73 to occur, and detecting formation of a complex between the antibody molecule and soluble CD73,
wherein the level of soluble CD73 is indicative of the responsiveness of the subject to the therapeutic agent.

35. A method of evaluating effectiveness of a therapeutic agent in a subject, wherein the subject has received or is going to receive administration of the therapeutic agent (e.g., one, two or all of a therapeutic anti-CD73 antibody molecule, a PD-1 inhibitor and an adenosine A2AR antagonist), comprising acquiring the level of soluble CD73 in the subject, e.g., at one or more time points prior to, during, or after the administration of the therapeutic agent, wherein acquiring the level of soluble CD73 comprises, e.g., contacting the subject, e.g., a biological sample (e.g., serum or plasma) from the subject, with the antibody molecule of any one of claims 1-25 under conditions that allow interaction of the antibody molecule and soluble CD73 to occur, and detecting formation of a complex between the antibody molecule and soluble CD73,
wherein the level of soluble CD73 is indicative of the effectiveness of the therapeutic agent in the subject.

36. The method of any one of claims 33-35, wherein the therapeutic anti-CD73 antibody molecule comprises:
(i) a VH comprising a VHCDR1 amino acid sequence of SEQ ID NO: 38, a VHCDR2 amino acid sequence of SEQ ID NO: 36, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;
(ii) a VH comprising a VHCDR1 amino acid sequence of SEQ ID NO: 72, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;
(iii) a VH comprising a VHCDR1 amino acid sequence of SEQ ID NO: 38, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;
(iv) a VH comprising a VHCDR1 amino acid sequence of SEQ ID NO: 137, a VHCDR2 amino acid sequence of SEQ ID NO: 136, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;
(v) a VH comprising a VHCDR1 amino acid sequence of SEQ ID NO: 137, a VHCDR2 amino acid sequence of SEQ ID NO: 146, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;
(vi) a VH comprising a VHCDR1 amino acid sequence of SEQ ID NO: 137, a VHCDR2 amino acid sequence of SEQ ID NO: 154, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50;
(vii) a VH comprising a VHCDR1 amino acid sequence of SEQ ID NO: 61, a VHCDR2 amino acid sequence of SEQ ID NO: 60, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;
(viii) a VH comprising a VHCDR1 amino acid sequence of SEQ ID NO: 4, a VHCDR2 amino acid sequence of SEQ ID NO: 26, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16;
(ix) a VH comprising a VHCDR1 amino acid sequence of SEQ ID NO: 4, a VHCDR2 amino acid sequence of SEQ ID NO: 2, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16, or
(x) a VH comprising a VHCDR1 amino acid sequence of SEQ ID NO: 163, a VHCDR2 amino acid sequence of SEQ ID NO: 162, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16.

37. The method of any one of claims 33-36, wherein the therapeutic anti-CD73 antibody molecule comprises:
(i) a heavy chain variable region comprising the amino acid sequence of SEQ ID NO: 44 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto);
(ii) a heavy chain variable region comprising the amino acid sequence of SEQ ID NO: 77 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto);
(iii) a heavy chain variable region comprising the amino acid sequence of SEQ ID NO: 84 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto);
(iv) a heavy chain variable region comprising the amino acid sequence of SEQ ID NO: 142 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto);
(v) a heavy chain variable region comprising the amino acid sequence of SEQ ID NO: 151 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto);
(vi) a heavy chain variable region comprising the amino acid sequence of SEQ ID NO: 159 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto);
(vii) a heavy chain variable region comprising the amino acid sequence of SEQ ID NO: 66 (or a sequence having at least about 85%, 90%, 95%, or 99% sequence identity thereto) and a light chain variable region comprising the amino acid sequence of SEQ ID NO: 21 (or a sequence having at least about 85%, 90%, 95%, or 99% sequence identity thereto);
(viii) a heavy chain variable region comprising the amino acid sequence of SEQ ID NO: 31 (or a sequence having at least about 85%, 90%, 95%, or 99% sequence identity thereto) and a light chain variable region comprising the amino acid sequence of SEQ ID NO: 21 (or a sequence having at least about 85%, 90%, 95%, or 99% sequence identity thereto);
(ix) a heavy chain variable region comprising the amino acid sequence of SEQ ID NO: 10 (or a sequence having at least about 85%, 90%, 95%, or 99% sequence identity thereto) and a light chain variable region comprising the amino acid sequence of SEQ ID NO: 21 (or a sequence having at least about 85%, 90%, 95%, or 99% sequence identity thereto); or
(x) a heavy chain variable region comprising the amino acid sequence of SEQ ID NO: 168 (or a sequence having at least about 85%, 90%, 95%, or 99% sequence identity thereto) and a light chain variable region comprising the amino acid sequence of SEQ ID NO: 21 (or a sequence having at least about 85%, 90%, 95%, or 99% sequence identity thereto).

38. The method of any one of claims 33-37, wherein the therapeutic anti-CD73 antibody molecule comprises:
(i) a heavy chain comprising the amino acid sequence of SEQ ID NO: 46 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto);
(ii) a heavy chain comprising the amino acid sequence of SEQ ID NO: 114 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto);
(iii) a heavy chain comprising the amino acid sequence of SEQ ID NO: 79 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto);
(iv) a heavy chain comprising the amino acid sequence of SEQ ID NO: 116 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto);
(v) a heavy chain comprising the amino acid sequence of SEQ ID NO: 86 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto);
(vi) a heavy chain comprising the amino acid sequence of SEQ ID NO: 117 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto);
(vii) a heavy chain comprising the amino acid sequence of SEQ ID NO: 68 (or a sequence having at least about 85%, 90%, 95%, or 99% sequence identity thereto) and a light chain comprising the amino acid sequence of SEQ ID NO: 23 (or a sequence having at least about 85%, 90%, 95%, or 99% sequence identity thereto);
(viii) a heavy chain comprising the amino acid sequence of SEQ ID NO: 115 (or a sequence having at least about 85%, 90%, 95%, or 99% sequence identity thereto) and a light chain comprising the amino acid sequence of SEQ ID NO: 23 (or a sequence having at least about 85%, 90%, 95%, or 99% sequence identity thereto);
(ix) a heavy chain comprising the amino acid sequence of SEQ ID NO: 33 (or a sequence having at least about 85%, 90%, 95%, or 99% sequence identity thereto) and a light chain comprising the amino acid sequence of SEQ ID NO: 23 (or a sequence having at least about 85%, 90%, 95%, or 99% sequence identity thereto);
(x) a heavy chain comprising the amino acid sequence of SEQ ID NO: 113 (or a sequence having at least about 85%, 90%, 95%, or 99% sequence identity thereto) and a light chain comprising the amino acid sequence of SEQ ID NO: 23 (or a sequence having at least about 85%, 90%, 95%, or 99% sequence identity thereto);
(xi) a heavy chain comprising the amino acid sequence of SEQ ID NO: 12 (or a sequence having at least about 85%, 90%, 95%, or 99% sequence identity thereto) and a light chain comprising the amino acid sequence of SEQ ID NO: 23 (or a sequence having at least about 85%, 90%, 95%, or 99% sequence identity thereto); or
(xii) a heavy chain comprising the amino acid sequence of SEQ ID NO: 112 (or a sequence having at least about 85%, 90%, 95%, or 99% sequence identity thereto) and a light chain comprising the amino acid sequence of SEQ ID NO: 23 (or a sequence having at least about 85%, 90%, 95%, or 99% sequence identity thereto).

39. The method of any one of claims 33-38, wherein the PD-1 inhibitor is a PD-1 inhibitor described herein.

40. The method of any one of claims 33-39, wherein the adenosine A2AR antagonist is an adenosine A2AR antagonist described herein.

41. A system for detecting soluble CD73 in a biological sample (e.g., serum or plasma) from a subject, comprising:
(i) the antibody molecule of any one of claims 1-25, e g, immobilized on a solid support, for contacting the biological sample to form a complex between the antibody molecule and soluble CD73 in the biological sample, and
(ii) a detecting reagent, e.g., a labeled anti-CD73 antibody molecule, for contacting the complex thereby detecting the soluble CD73 in the biological sample.

42. The method of any one of claims 33-40, wherein in addition to administration of said therapeutic agent, (e.g., one, two or all of a therapeutic anti-CD73 antibody molecule, a PD-1 inhibitor and an adenosine A2AR antagonist), one or more of the following are administered:
(i) a CTLA-4 inhibitor, optionally wherein the CTLA-4 inhibitor is Ipilimumab or Tremelimumab;
(ii) a TIM-3 inhibitor, optionally wherein the TIM-3 inhibitor is selected from the group consisting of MGB453, TSR-022, and LY3321367;
(iii) a LAG-3 inhibitor, optionally wherein the LAG-3 inhibitor is selected from the group consisting of LAG525, BMS-986016, TSR-033, MK-4280 and REGN3767;
(iv) a GITR agonist, optionally wherein the GITR agonist is selected from the group consisting of GWN323, BMS-986156, MK-4166, MK-1248, TRX518, INCAGN1876, AMG 228, and INBRX-110;
(v) an anti-CD3 multispecific antibody molecule, optionally wherein the anti-CD3 multispecific antibody molecule is an anti-CD3×anti-CD123 bispecific antibody molecule (e.g., XENP14045), or an anti-CD3×anti-CD20 bispecific antibody molecule (e.g., XENP13676);
(vi) a cytokine molecule, optionally wherein the cytokine molecule is IL-15 complexed with a soluble form of IL-15 receptor alpha (IL-15Ra);
(vii) a STING agonist;
(viii) a macrophage colony-stimulating factor (M-CSF) inhibitor, optionally wherein the M-CSF inhibitor is MCS110;
(ix) a CSF-1R inhibitor, optionally wherein the CSF-1R inhibitor is BLZ945;
(x) an inhibitor of indoleamine 2,3-dioxygenase (IDO) and/or tryptophan 2,3-dioxygenase (TDO);
(xi) a TGF-β inhibitor;
(xii) an oncolytic vaccine;
(xiii) a chimeric antigen receptor (CAR) T-cell therapy, optionally wherein the CAR T-cell therapy is CTL019; or
(xiv) a compound disclosed in Table 18.

43. The method of any one of claim 33-40 or 42, wherein the therapeutic anti-CD73 antibody molecule is administered at a dose of about 100 mg to 1600 mg, about 100 mg to 1400 mg, about 100 mg to 1200 mg, about 100 mg to 1000 mg, about 100 mg to 800 mg, about 100 mg to 600 mg, about 100 mg to 400 mg, about 100 mg to 200 mg, or about 100 mg, about 180 mg, or about 200 mg, once every two weeks (Q2W), optionally wherein the antibody molecule is administered at a dose of at least about 180 mg Q2W.

44. The method of any one of claim 33-40 or 42-43, wherein the therapeutic anti-CD73 antibody molecule is administered, e.g., intravenously, at a dose of about 5 mg to 100 mg, about 100 mg to 500 mg, about 500 mg to 1000 mg, about 1000 mg to 1500 mg, about 1500 mg to 2000 mg, about 2000 mg to 2500 mg, about 2500 mg to 3000 mg, about 3000 mg to 3500 mg, or about 3500 mg to 4000 mg, e.g., at a dose of about 6 mg, about 20 mg, about 60 mg, about 200 mg, about 600 mg, about 1200 mg, about 2400 mg, about 3000 mg, or about 3600 mg, e.g., once every week (QW), once every two weeks (Q2W), or once every four weeks (Q4W), e.g., Q2W.

45. The method of any one of claim 33-40 or 42-44, wherein the therapeutic anti-CD73 antibody molecule is administered in combination with a PD-1 inhibitor, optionally wherein the PD-1 inhibitor is selected from the group consisting of PDR001, Nivolumab, Pembrolizumab, Pidilizumab, MEDI0680, REGN2810, TSR-042, PF-06801591, and AMP-224, optionally wherein:
the PD-1 inhibitor is an anti-PD-1 antibody molecule, wherein the anti-PD-1 antibody molecule is administered, e.g., intravenously, at a dose of about 250 mg to 350 mg, about 350 mg to 450 mg, or about 450 mg to 550 mg, e.g., at a dose of about 300 mg or about 400 mg, e.g., once every three weeks (Q3W) or once every four weeks (Q4W), e.g., at a dose of about 300 mg Q3W, or at a dose of about 400 mg Q4W, optionally wherein:
the anti-CD73 antibody molecule is administered, e.g., intravenously, at a dose of about 5 mg to 100 mg, e.g., 20 mg, Q2W, and the anti-PD-1 antibody molecule is administered, e.g., intravenously, at a dose of about 350 mg to 450 mg, e.g., 400 mg, Q4W,
the anti-CD73 antibody molecule is administered, e.g., intravenously, at a dose of about 5 mg to 100 mg, e.g., 60 mg, Q2W, and the anti-PD-1 antibody molecule is administered, e.g., intravenously, at a dose of about 350 mg to 450 mg, e.g., 400 mg, Q4W,
the anti-CD73 antibody molecule is administered, e.g., intravenously, at a dose of about 100 mg to 500 mg, e.g., 200 mg, Q2W, and the anti-PD-1 antibody molecule is administered, e.g., intravenously, at a dose of about 350 mg to 450 mg, e.g., 400 mg, Q4W,
the anti-CD73 antibody molecule is administered, e.g., intravenously, at a dose of about 500 mg to 1000 mg, e.g., 600 mg, Q2W, and the anti-PD-1 antibody molecule is administered, e.g., intravenously, at a dose of about 350 mg to 450 mg, e.g., 400 mg, Q4W,
the anti-CD73 antibody molecule is administered, e.g., intravenously, at a dose of about 1000 mg to 1500 mg, e.g., 1200 mg, Q2W, and the anti-PD-1 antibody molecule is administered, e.g., intravenously, at a dose of about 350 mg to 450 mg, e.g., 400 mg, Q4W,
the anti-CD73 antibody molecule is administered, e.g., intravenously, at a dose of about 2000 mg to 2500 mg, e.g., 2400 mg, Q2W, and the anti-PD-1 antibody molecule is administered, e.g., intravenously, at a dose of about 350 mg to 450 mg, e.g., 400 mg, Q4W,
the anti-CD73 antibody molecule is administered, e.g., intravenously, at a dose of about 3000 mg to 3500 mg, e.g., 3000 mg, Q2W, and the anti-PD-1 antibody molecule is administered, e.g., intravenously, at a dose of about 350 mg to 450 mg, e.g., 400 mg, Q4W, or
the anti-CD73 antibody molecule is administered, e.g., intravenously, at a dose of about 3500 mg to 4000 mg, e.g., 3600 mg, Q2W, and the anti-PD-1 antibody molecule is administered, e.g., intravenously, at a dose of about 350 mg to 450 mg, e.g., 400 mg, Q4W.

46. The method of any one of claim 33-40 or 42-45, wherein the therapeutic anti-CD73 antibody molecule is administered in combination with an adenosine A2AR antagonist, optionally wherein:
(i) the adenosine A2AR antagonist is selected from the group consisting of PBF509, CPI444, AZD4635, Vipadenant, GBV-2034, and AB928; or
(ii) the adenosine A2AR antagonist is selected from the group consisting of 5-bromo-2,6-di-(1H-pyrazol-1-yl)pyrimidine-4-amine; (S)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; (R)-7-(5-methylfuran-2-yl)-3-((6-(((tetrahydrofuran-3-yl)oxy)methyl)pyridin-2-yl)methyl)-3H-[1,2,3]triazolo[4,5-c]pyrimidin-5-amine, or racemate thereof; 7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; and 6-(2-chloro-6-methylpyridin-4-yl)-5-(4-fluorophenyl)-1,2,4-triazin-3-amine, optionally wherein:
the adenosine A2AR antagonist is administered, e.g., orally, at a dose of about 20 mg to 60 mg, about 60 mg to 100 mg, about 100 mg to 140 mg, about 140 mg to 180 mg, about 180 mg to 220 mg, about 220 mg to 260 mg, about 260 mg to 300 mg, about 300 mg to 340 mg, about 340 mg to 380 mg, about 380 mg to 480 mg, about 480 mg to 580 mg, or about 580 mg to 680 mg, e.g., at a dose of about 40 mg, about 80 mg, about 160 mg, about 320 mg, about 480 mg, or about 620 mg, e.g., once a day (QD), twice a day (BID), or three times a day (TID), e.g., BID, optionally wherein:
the anti-CD73 antibody molecule is administered, e.g., intravenously, at a dose of about 5 mg to 100 mg, e.g., 20 mg, Q2W, and the adenosine A2AR antagonist is administered, e.g., orally, at a dose of about 20 mg to 60 mg, e.g., 40 mg, BID,
the anti-CD73 antibody molecule is administered, e.g., intravenously, at a dose of about 5 mg to 100 mg, e.g., 60 mg, Q2W, and the adenosine A2AR antagonist is administered, e.g., orally, at a dose of about 20 mg to 60 mg, e.g., 40 mg, BID,
the anti-CD73 antibody molecule is administered, e.g., intravenously, at a dose of about 100 mg to 500 mg, e.g., 200 mg, Q2W, and the adenosine A2AR antagonist is administered, e.g., orally, at a dose of about 60 mg to 100 mg, e.g., 80 mg, BID,
the anti-CD73 antibody molecule is administered, e.g., intravenously, at a dose of about 100 mg to 500 mg, e.g., 200 mg, Q2W, and the adenosine A2AR antagonist is administered, e.g., orally, at a dose of about 140 mg to 180 mg, e.g., 160 mg, BID,
the anti-CD73 antibody molecule is administered, e.g., intravenously, at a dose of about 500 mg to 1000 mg, e.g., 600 mg, Q2W, and the adenosine A2AR antagonist is administered, e.g., orally, at a dose of about 140 mg to 180 mg, e.g., 160 mg, BID,
the anti-CD73 antibody molecule is administered, e.g., intravenously, at a dose of about 1000 mg to 1500 mg, e.g., 1200 mg, Q2W, and the adenosine A2AR antagonist is administered, e.g., orally, at a dose of about 140 mg to 180 mg, e.g., 160 mg, BID,
the anti-CD73 antibody molecule is administered, e.g., intravenously, at a dose of about 2000 mg to 2500 mg, e.g., 2400 mg, Q2W, and the adenosine A2AR antagonist is administered, e.g., orally, at a dose of about 140 mg to 180 mg, e.g., 160 mg, BID,
the anti-CD73 antibody molecule is administered, e.g., intravenously, at a dose of about 2000 mg to 2500 mg, e.g., 2400 mg, Q2W, and the adenosine A2AR antagonist is administered, e.g., orally, at a dose of about 300 mg to 340 mg, e.g., 320 mg, BID,
the anti-CD73 antibody molecule is administered, e.g., intravenously, at a dose of about 2000 mg to 2500 mg, e.g., 2400 mg, Q2W, and the adenosine A2AR antagonist is administered, e.g., orally, at a dose of about 480 mg to 580 mg, e.g., 480 mg, BID,
the anti-CD73 antibody molecule is administered, e.g., intravenously, at a dose of about 2000 mg to 2500 mg, e.g., 2400 mg, Q2W, and the adenosine A2AR antagonist is administered, e.g., orally, at a dose of about 580 mg to 680 mg, e.g., 620 mg, BID,
the anti-CD73 antibody molecule is administered, e.g., intravenously, at a dose of about 3000 mg to 3500 mg, e.g., 3000 mg, Q2W, and the adenosine A2AR antagonist is administered, e.g., orally, at a dose of about 300 mg to 340 mg, e.g., 320 mg, BID,
the anti-CD73 antibody molecule is administered, e.g., intravenously, at a dose of about 3000 mg to 3500 mg, e.g., 3000 mg, Q2W, and the adenosine A2AR antagonist is administered, e.g., orally, at a dose of about 480 mg to 580 mg, e.g., 480 mg, BID,
the anti-CD73 antibody molecule is administered, e.g., intravenously, at a dose of about 3000 mg to 3500 mg, e.g., 3000 mg, Q2W, and the adenosine A2AR antagonist is administered, e.g., orally, at a dose of about 580 mg to 680 mg, e.g., 620 mg, BID,
the anti-CD73 antibody molecule is administered, e.g., intravenously, at a dose of about 3500 mg to 4000 mg, e.g., 3600 mg, Q2W, and the adenosine A2AR antagonist is administered, e.g., orally, at a dose of about 300 mg to 340 mg, e.g., 320 mg, BID,
the anti-CD73 antibody molecule is administered, e.g., intravenously, at a dose of about 3500 mg to 4000 mg, e.g., 3600 mg, Q2W, and the adenosine A2AR antagonist is administered, e.g., orally, at a dose of about 480 mg to 580 mg, e.g., 480 mg, BID, or
the anti-CD73 antibody molecule is administered, e.g., intravenously, at a dose of about 3500 mg to 4000 mg, e.g., 3600 mg, Q2W, and the adenosine A2AR antagonist is administered, e.g., orally, at a dose of about 580 mg to 680 mg, e.g., 620 mg, BID.

47. The method of any one of claim 33-40 or 42-46, wherein the therapeutic anti-CD73 antibody molecule is administered in combination with a PD-1 inhibitor and an adenosine A2AR antagonist, optionally wherein:
the PD-1 inhibitor is an anti-PD-1 antibody molecule, wherein the anti-PD-1 antibody molecule is administered, e.g., intravenously, at a dose of about 250 mg to 350 mg, about 350 mg to 450 mg, or about 450 mg to 550 mg, e.g., at a dose of about 300 mg or about 400 mg, e.g., once every three weeks (Q3W) or once every four weeks (Q4W), e.g., at a dose of about 300 mg Q3W, or at a dose of about 400 mg Q4W, optionally wherein:
the adenosine A2AR antagonist is administered, e.g., orally, at a dose of about 20 mg to 60 mg, about 60 mg to 100 mg, about 100 mg to 140 mg, about 140 mg to 180 mg, about 180 mg to 220 mg, about 220 mg to 260 mg, about 260 mg to 300 mg, about 300 mg to 340 mg, or about 340 mg to 380 mg, e.g., at a dose of about 40 mg, about 80 mg, about 160 mg, or about 320 mg, e.g., once a day (QD), twice a day (BID), or three times a day (TID), e.g., BID, optionally wherein:
the anti-CD73 antibody molecule is administered, e.g., intravenously, at a dose of about 5 mg to 100 mg, e.g., 20 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 20 mg to 60 mg, e.g., 40 mg, BID,
the anti-CD73 antibody molecule is administered, e.g., intravenously, at a dose of about 5 mg to 100 mg, e.g., 60 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 20 mg to 60 mg, e.g., 40 mg, BID,
the anti-CD73 antibody molecule is administered, e.g., intravenously, at a dose of about 100 mg to 500 mg, e.g., 200 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 60 mg to 100 mg, e.g., 80 mg, BID,
the anti-CD73 antibody molecule is administered, e.g., intravenously, at a dose of about 100 mg to 500 mg, e.g., 200 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 140 mg to 180 mg, e.g., 160 mg, BID,
the anti-CD73 antibody molecule is administered, e.g., intravenously, at a dose of about 500 mg to 1000 mg, e.g., 600 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 140 mg to 180 mg, e.g., 160 mg, BID,
the anti-CD73 antibody molecule is administered, e.g., intravenously, at a dose of about 1000 mg to 1500 mg, e.g., 1200 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 140 mg to 180 mg, e.g., 160 mg, BID,
the anti-CD73 antibody molecule is administered, e.g., intravenously, at a dose of about 2000 mg to 2500 mg, e.g., 2400 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 140 mg to 180 mg, e.g., 160 mg, BID,
the anti-CD73 antibody molecule is administered, e.g., intravenously, at a dose of about 2000 mg to 2500 mg, e.g., 2400 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 300 mg to 340 mg, e.g., 320 mg, BID,
the anti-CD73 antibody molecule is administered, e.g., intravenously, at a dose of about 2000 mg to 2500 mg, e.g., 2400 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 480 mg to 580 mg, e.g., 480 mg, BID,
the anti-CD73 antibody molecule is administered, e.g., intravenously, at a dose of about 2000 mg to 2500 mg, e.g., 2400 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 580 mg to 680 mg, e.g., 620 mg, BID,
the anti-CD73 antibody molecule is administered, e.g., intravenously, at a dose of about 3000 mg to 3500 mg, e.g., 3000 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 300 mg to 340 mg, e.g., 320 mg, BID,
the anti-CD73 antibody molecule is administered, e.g., intravenously, at a dose of about 3000 mg to 3500 mg, e.g., 3000 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 480 mg to 580 mg, e.g., 480 mg, BID,
the anti-CD73 antibody molecule is administered, e.g., intravenously, at a dose of about 3000 mg to 3500 mg, e.g., 3000 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 580 mg to 680 mg, e.g., 620 mg, BID,
the anti-CD73 antibody molecule is administered, e.g., intravenously, at a dose of about 3500 mg to 4000 mg, e.g., 3600 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 300 mg to 340 mg, e.g., 320 mg, BID,
the anti-CD73 antibody molecule is administered, e.g., intravenously, at a dose of about 3500 mg to 4000 mg, e.g., 3600 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 480 mg to 580 mg, e.g., 480 mg, BID, or
the anti-CD73 antibody molecule is administered, e.g., intravenously, at a dose of about 3500 mg to 4000 mg, e.g., 3600 mg, Q2W, the anti-PD-1 antibody molecule is administered, e.g., intravenously, at a dose of about 350 mg to 450 mg, e.g., 400 mg, Q4W, and the adenosine A2AR antagonist is administered, e.g., orally, at a dose of about 580 mg to 680 mg, e.g., 620 mg, BID.


SEQUENCE LISTING (922 sequences)

SEQ ID NO: 1 - protein, 10 aa, Artificial Sequence
  source: /note=“Description of Artificial Sequence: Synthetic peptide”
GGSISGRYWS

SEQ ID NO: 2 - protein, 16 aa, Artificial Sequence
  source: /note=“Description of Artificial Sequence: Synthetic peptide”
YIYGTGSTNYNPSLKS

SEQ ID NO: 3 - protein, 13 aa, Artificial Sequence
  source: /note=“Description of Artificial Sequence: Synthetic peptide”
ESQESPYNNWFDP

SEQ ID NO: 4 - protein, 5 aa, Artificial Sequence
  source: /note=“Description of Artificial Sequence: Synthetic peptide”
GRYWS

SEQ ID NO: 5 - protein, 7 aa, Artificial Sequence
  source: /note=“Description of Artificial Sequence: Synthetic peptide”
GGSISGR

SEQ ID NO: 6 - protein, 5 aa, Artificial Sequence
  source: /note=“Description of Artificial Sequence: Synthetic peptide”
YGTGS

SEQ ID NO: 7 - protein, 8 aa, Artificial Sequence
  source: /note=“Description of Artificial Sequence: Synthetic peptide”
GGSISGRY

SEQ ID NO: 8 - protein, 7 aa, Artificial Sequence
  source: /note=“Description of Artificial Sequence: Synthetic peptide”
IYGTGST

SEQ ID NO: 9 - protein, 15 aa, Artificial Sequence
  source: /note=“Description of Artificial Sequence: Synthetic peptide”
ARESQESPYNNWFDP

SEQ ID NO: 10 - protein, 121 aa, Artificial Sequence
  source: /note=“Description of Artificial Sequence: Synthetic polypeptide”
QVQLQESGPGLVKPSETLSLTCTVSGGSISGRYWSWIRQPPGKGLEWIGYIYGTGSTNYNPSLKSRVTISVDTSKNQFSLKLSSVTAADTAVYYCARESQESPYNNWFDPWGQGTLVTVSS

SEQ ID NO: 11 - DNA, 363 bp, Artificial Sequence
  source: /note=“Description of Artificial Sequence: Synthetic polynucleotide”
caagtgcagctgcaggaatctggccctggcctggtcaagccctccgagacactgtccctgacctgcaccgtgtccggcggctccatctccggccggtactggtcttggatccggcagcctcccggcaagggcctggaatggatcggctacatctacggcaccggctccaccaactacaaccccagcctgaagtccagagtgaccatctccgtggacacctccaagaaccagttctccctgaagctgtcctccgtgaccgccgctgacaccgccgtgtactactgcgccagagagtcccaggaatccccttacaacaattggttcgacccctggggccagggcaccctggtcaccgtgtcctct

SEQ ID NO: 12 - protein, 448 aa, Artificial Sequence
  source: /note=“Description of Artificial Sequence: Synthetic polypeptide”
  VARIANT (448)..(448): /replace=“ ”
  SITE (1)..(448): /note=“Variant residues given in the sequence have no preference with respect to those in the annotations for variant positions”
QVQLQESGPGLVKPSETLSLTCTVSGGSISGRYWSWIRQPPGKGLEWIGYIYGTGSTNYNPSLKSRVTISVDTSKNQFSLKLSSVTAADTAVYYCARESQESPYNNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - DNA, 1344 bp, Artificial Sequence
  source: /note=“Description of Artificial Sequence: Synthetic polynucleotide”
  misc_feature (1342)..(1344): /note=“This region may or may not be present in its entirety”
  source: /note=“See specification as filed for detailed description of substitutions and preferred embodiments”
caagtgcagctgcaggaatctggccctggcctggtcaagccctccgagacactgtccctgacctgcaccgtgtccggcggctccatctccggccggtactggtcttggatccggcagcctcccggcaagggcctggaatggatcggctacatctacggcaccggctccaccaactacaaccccagcctgaagtccagagtgaccatctccgtggacacctccaagaaccagttctccctgaagctgtcctccgtgaccgccgctgacaccgccgtgtactactgcgccagagagtcccaggaatccccttacaacaattggttcgacccctggggccagggcaccctggtcaccgtgtcctctgcttccaccaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgccctccttgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggcaag

SEQ ID NO: 14 - protein, 11 aa, Artificial Sequence
  source: /note=“Description of Artificial Sequence: Synthetic peptide”
RASQGISSWLA

SEQ ID NO: 15 - protein, 7 aa, Artificial Sequence
  source: /note=“Description of Artificial Sequence: Synthetic peptide”
AASSLQS

SEQ ID NO: 16 - protein, 9 aa, Artificial Sequence
  source: /note=“Description of Artificial Sequence: Synthetic peptide”
QQGNSFPRT

SEQ ID NO: 17 - protein, 7 aa, Artificial Sequence
  source: /note=“Description of Artificial Sequence: Synthetic peptide”
SQGISSW

SEQ ID NO: 18 - protein, 3 aa, Artificial Sequence
  source: /note=“Description of Artificial Sequence: Synthetic peptide”
AAS

SEQ ID NO: 19 - protein, 6 aa, Artificial Sequence
  source: /note=“Description of Artificial Sequence: Synthetic peptide”
GNSFPR

SEQ ID NO: 20 - protein, 6 aa, Artificial Sequence
  source: /note=“Description of Artificial Sequence: Synthetic peptide”
QGISSW

SEQ ID NO: 21 - protein, 107 aa, Artificial Sequence
  source: /note=“Description of Artificial Sequence: Synthetic polypeptide”
DIQMTQSPSSVSASVGDRVTITCRASQGISSWLAWYQQKPGKAPKLLIYAASSLQSGVPSRFSGSGSGTDFTLTISSLQPEDFATYYCQQGNSFPRTFGGGTKVEIK

SEQ ID NO: 22 - DNA, 321 bp, Artificial Sequence
  source: /note=“Description of Artificial Sequence: Synthetic polynucleotide”
gacatccagatgacccagagcccctcctccgtgtccgcctccgtgggcgacagagtgaccatcacctgtcgggcctcccagggcatctccagctggctggcctggtatcagcagaagcccggcaaggcccctaagctgctgatctacgccgcctccagcctgcagtccggcgtgccctccagattctccggctctggctccggcaccgacttcaccctgaccatcagctccctgcagcccgaggacttcgccacctactactgccagcagggcaactccttccctcggaccttcggcggaggcaccaaagtggaaatcaag

SEQ ID NO: 23 - protein, 214 aa, Artificial Sequence
  source: /note=“Description of Artificial Sequence: Synthetic polypeptide”
DIQMTQSPSSVSASVGDRVTITCRASQGISSWLAWYQQKPGKAPKLLIYAASSLQSGVPSRFSGSGSGTDFTLTISSLQPEDFATYYCQQGNSFPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - DNA, 642 bp, Artificial Sequence
  source: /note=“Description of Artificial Sequence: Synthetic polynucleotide”
gacatccagatgacccagagcccctcctccgtgtccgcctccgtgggcgacagagtgaccatcacctgtcgggcctcccagggcatctccagctggctggcctggtatcagcagaagcccggcaaggcccctaagctgctgatctacgccgcctccagcctgcagtccggcgtgccctccagattctccggctctggctccggcaccgacttcaccctgaccatcagctccctgcagcccgaggacttcgccacctactactgccagcagggcaactccttccctcggaccttcggcggaggcaccaaagtggaaatcaagcgtacggtggccgctccctccgtgttcatcttcccaccctccgacgagcagctgaagtccggcaccgcctccgtcgtgtgcctgctgaacaacttctaccctcgcgaggccaaagtgcagtggaaagtggacaacgccctgcagtccggcaactcccaggaatccgtcaccgagcaggactccaaggacagcacctactccctgtcctccaccctgaccctgtccaaggccgactacgagaagcacaaagtgtacgcctgcgaagtgacccaccagggcctgtccagccccgtgaccaagtccttcaaccggggcgagtgc

SEQ ID NO: 25 - protein, 10 aa, Artificial Sequence
  source: /note=“Description of Artificial Sequence: Synthetic peptide”
GGSIEGRYWS

SEQ ID NO: 26 - protein, 16 aa, Artificial Sequence
  source: /note=“Description of Artificial Sequence: Synthetic peptide”
YIYGSGSTKYNPSLKS

SEQ ID NO: 27 - protein, 7 aa, Artificial Sequence
  source: /note=“Description of Artificial Sequence: Synthetic peptide”
GGSIEGR

SEQ ID NO: 28 - protein, 5 aa, Artificial Sequence
  source: /note=“Description of Artificial Sequence: Synthetic peptide”
YGSGS

SEQ ID NO: 29 - protein, 8 aa, Artificial Sequence
  source: /note=“Description of Artificial Sequence: Synthetic peptide”
GGSIEGRY

SEQ ID NO: 30 - protein, 7 aa, Artificial Sequence
  source: /note=“Description of Artificial Sequence: Synthetic peptide”
IYGSGST

SEQ ID NO: 31 - protein, 121 aa, Artificial Sequence
  source: /note=“Description of Artificial Sequence: Synthetic polypeptide”
QVQLQESGPGLVKPSETLSLTCTVSGGSIEGRYWSWIRQPPGKGLEWIGYIYGSGSTKYNPSLKSRVTISVDTSKNQFSLKLSSVTAADTAVYYCARESQESPYNNWFDPWGQGTLVTVSS

SEQ ID NO: 32 - DNA, 363 bp, Artificial Sequence
  source: /note=“Description of Artificial Sequence: Synthetic polynucleotide”
caagtgcagctgcaggaatctggccctggcctggtcaagccctccgagacactgtccctgacctgcaccgtgtccggcggctctatcgagggccggtactggtcctggatccggcagcctcctggcaagggcctggaatggatcggctacatctacggctccggctccaccaagtacaaccccagcctgaagtccagagtgaccatctccgtggacacctccaagaaccagttctccctgaagctgtcctccgtgaccgccgctgacaccgccgtgtactactgcgccagagagtcccaggaatccccttacaacaattggttcgacccctggggccagggcaccctggtcaccgtgtcctct

SEQ ID NO: 33 - protein, 448 aa, Artificial Sequence
  source: /note=“Description of Artificial Sequence: Synthetic polypeptide”
  VARIANT (448)..(448): /replace=“ ”
  SITE (1)..(448): /note=“Variant residues given in the sequence have no preference with respect to those in the annotations for variant positions”
QVQLQESGPGLVKPSETLSLTCTVSGGSIEGRYWSWIRQPPGKGLEWIGYIYGSGSTKYNPSLKSRVTISVDTSKNQFSLKLSSVTAADTAVYYCARESQESPYNNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - DNA, 1344 bp, Artificial Sequence
  source: /note=“Description of Artificial Sequence: Synthetic polynucleotide”
  misc_feature (1342)..(1344): /note=“This region may or may not be present in its entirety”
  source: /note=“See specification as filed for detailed description of substitutions and preferred embodiments”
caagtgcagctgcaggaatctggccctggcctggtcaagccctccgagacactgtccctgacctgcaccgtgtccggcggctctatcgagggccggtactggtcctggatccggcagcctcctggcaagggcctggaatggatcggctacatctacggctccggctccaccaagtacaaccccagcctgaagtccagagtgaccatctccgtggacacctccaagaaccagttctccctgaagctgtcctccgtgaccgccgctgacaccgccgtgtactactgcgccagagagtcccaggaatccccttacaacaattggttcgacccctggggccagggcaccctggtcaccgtgtcctctgcctccaccaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgccctccttgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggcaag

SEQ ID NO: 35 - protein, 10 aa, Artificial Sequence
  source: /note=“Description of Artificial Sequence: Synthetic peptide”
GFTFHRYAMS

SEQ ID NO: 36 - protein, 17 aa, Artificial Sequence
  source: /note=“Description of Artificial Sequence: Synthetic peptide”
AISGSGMNTYYADSVKG

SEQ ID NO: 37 - protein, 15 aa, Artificial Sequence
  source: /note=“Description of Artificial Sequence: Synthetic peptide”
GGLYGSGSYLSDFDL

SEQ ID NO: 38 - protein, 5 aa, Artificial Sequence
  source: /note=“Description of Artificial Sequence: Synthetic peptide”
RYAMS

SEQ ID NO: 39 - protein, 7 aa, Artificial Sequence
  source: /note=“Description of Artificial Sequence: Synthetic peptide”
GFTFHRY

SEQ ID NO: 40 - protein, 6 aa, Artificial Sequence
  source: /note=“Description of Artificial Sequence: Synthetic peptide”
SGSGMN

SEQ ID NO: 41 - protein, 8 aa, Artificial Sequence
  source: /note=“Description of Artificial Sequence: Synthetic peptide”
GFTFHRYA

SEQ ID NO: 42 - protein, 8 aa, Artificial Sequence
  source: /note=“Description of Artificial Sequence: Synthetic peptide”
ISGSGMNT

SEQ ID NO: 43 - protein, 17 aa, Artificial Sequence
  source: /note=“Description of Artificial Sequence: Synthetic peptide”
ARGGLYGSGSYLSDFDL

SEQ ID NO: 44 - protein, 124 aa, Artificial Sequence
  source: /note=“Description of Artificial Sequence: Synthetic polypeptide”
EVQLLESGGGLVQPGGSLRLSCAASGFTFHRYAMSWVRQAPGKGLEWVSAISGSGMNTYYADSVKGRFTISRDNSKNTLYLQMNSLRAEDTAVYYCARGGLYGSGSYLSDFDLWGRGTLVTVSS

SEQ ID NO: 45 - DNA, 372 bp, Artificial Sequence
  source: /note=“Description of Artificial Sequence: Synthetic polynucleotide”
gaagtgcagctgctggaatctggcggcggactggtgcagcctggcggctccctgagactgtcttgcgccgcctccggcttcaccttccacagatacgccatgtcctgggtccgacaggcccctggcaagggcctggagtgggtgtccgccatctccggctccggcatgaacacctactacgccgactccgtgaagggccggttcaccatctcccgggacaactccaagaacaccctgtacctgcagatgaactccctgcgggccgaggacaccgccgtgtactactgtgccagaggcggcctgtacggctccggctcctacctgtccgacttcgacctgtggggcagaggcaccctggtcaccgtgtcctcc

SEQ ID NO: 46 - protein, 451 aa, Artificial Sequence
  source: /note=“Description of Artificial Sequence: Synthetic polypeptide”
  VARIANT (451)..(451): /replace=“ ”
  SITE (1)..(451): /note=“Variant residues given in the sequence have no preference with respect to those in the annotations for variant positions”
EVQLLESGGGLVQPGGSLRLSCAASGFTFHRYAMSWVRQAPGKGLEWVSAISGSGMNTYYADSVKGRFTISRDNSKNTLYLQMNSLRAEDTAVYYCARGGLYGSGSYLSDFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - DNA, 1353 bp, Artificial Sequence
  source: /note=“Description of Artificial Sequence: Synthetic polynucleotide”
  misc_feature (1351)..(1353): /note=“This region may or may not be present in its entirety”
  source: /note=“See specification as filed for detailed description of substitutions and preferred embodiments”
gaagtgcagctgctggaatctggcggcggactggtgcagcctggcggctccctgagactgtcttgcgccgcctccggcttcaccttccacagatacgccatgtcctgggtccgacaggcccctggcaagggcctggagtgggtgtccgccatctccggctccggcatgaacacctactacgccgactccgtgaagggccggttcaccatctcccgggacaactccaagaacaccctgtacctgcagatgaactccctgcgggccgaggacaccgccgtgtactactgtgccagaggcggcctgtacggctccggctcctacctgtccgacttcgacctgtggggcagaggcaccctggtcaccgtgtcctccgcctccacaaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgccctccttgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggcaag

SEQ ID NO: 48 - protein, 11 aa, Artificial Sequence
  source: /note=“Description of Artificial Sequence: Synthetic peptide”
RASQSVGSNLA

SEQ ID NO: 49 - protein, 7 aa, Artificial Sequence
  source: /note=“Description of Artificial Sequence: Synthetic peptide”
GASTRAT

SEQ ID NO: 50 - protein, 9 aa, Artificial Sequence
  source: /note=“Description of Artificial Sequence: Synthetic peptide”
QQHNAFPYT

SEQ ID NO: 51 - protein, 7 aa, Artificial Sequence
  source: /note=“Description of Artificial Sequence: Synthetic peptide”
SQSVGSN

SEQ ID NO: 52 - protein, 3 aa, Artificial Sequence
  source: /note=“Description of Artificial Sequence: Synthetic peptide”
GAS

SEQ ID NO: 53 - protein, 6 aa, Artificial Sequence
  source: /note=“Description of Artificial Sequence: Synthetic peptide”
HNAFPY

SEQ ID NO: 54 - protein, 6 aa, Artificial Sequence
  source: /note=“Description of Artificial Sequence: Synthetic peptide”
QSVGSN

SEQ ID NO: 55 - protein, 107 aa, Artificial Sequence
  source: /note=“Description of Artificial Sequence: Synthetic polypeptide”
EIVLTQSPATLSVSPGERATLSCRASQSVGSNLAWYQQKPGQAPRLLIYGASTRATGIPARFSGSGSGTEFTLTISSLQSEDFAVYYCQQHNAFPYTFGGGTKVEIK

SEQ ID NO: 56 - DNA, 321 bp, Artificial Sequence
  source: /note=“Description of Artificial Sequence: Synthetic polynucleotide”
gagatcgtgctgacacagtcccctgccaccctgtctgtgtctcccggcgagagagccaccctgagctgcagagcctcccagtccgtgggctccaacctggcctggtatcagcagaagcccggccaggcccctcggctgctgatctacggcgcctctaccagagccaccggcatccctgccagattctccggctctggctccggcaccgagttcaccctgaccatctccagcctgcagtccgaggacttcgccgtgtactactgccagcagcacaacgccttcccttacaccttcggcggaggcaccaaagtggaaatcaag

SEQ ID NO: 57 - protein, 214 aa, Artificial Sequence
  source: /note=“Description of Artificial Sequence: Synthetic polypeptide”
EIVLTQSPATLSVSPGERATLSCRASQSVGSNLAWYQQKPGQAPRLLIYGASTRATGIPARFSGSGSGTEFTLTISSLQSEDFAVYYCQQHNAFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - DNA, 642 bp, Artificial Sequence
  source: /note=“Description of Artificial Sequence: Synthetic polynucleotide”
gagatcgtgctgacacagtcccctgccaccctgtctgtgtctcccggcgagagagccaccctgagctgcagagcctcccagtccgtgggctccaacctggcctggtatcagcagaagcccggccaggcccctcggctgctgatctacggcgcctctaccagagccaccggcatccctgccagattctccggctctggctccggcaccgagttcaccctgaccatctccagcctgcagtccgaggacttcgccgtgtactactgccagcagcacaacgccttcccttacaccttcggcggaggcaccaaagtggaaatcaagcgtacggtggccgctccctccgtgttcatcttcccaccctccgacgagcagctgaagtccggcaccgcctccgtcgtgtgcctgctgaacaacttctaccctcgcgaggccaaagtgcagtggaaagtggacaacgccctgcagtccggcaactcccaggaatccgtcaccgagcaggactccaaggacagcacctactccctgtcctccaccctgaccctgtccaaggccgactacgagaagcacaaagtgtacgcctgcgaagtgacccaccagggcctgtccagccccgtgaccaagtccttcaaccggggcgagtgc

SEQ ID NO: 59 - protein, 10 aa, Artificial Sequence
  source: /note=“Description of Artificial Sequence: Synthetic peptide”
GGSIERYYWS

SEQ ID NO: 60 - protein, 16 aa, Artificial Sequence
  source: /note=“Description of Artificial Sequence: Synthetic peptide”
YIYGRGSTNYNPSLKS

SEQ ID NO: 61 - protein, 5 aa, Artificial Sequence
  source: /note=“Description of Artificial Sequence: Synthetic peptide”
RYYWS

SEQ ID NO: 62 - protein, 7 aa, Artificial Sequence
  source: /note=“Description of Artificial Sequence: Synthetic peptide”
GGSIERY

SEQ ID NO: 63 - protein, 5 aa, Artificial Sequence
  source: /note=“Description of Artificial Sequence: Synthetic peptide”
YGRGS

SEQ ID NO: 64 - protein, 8 aa, Artificial Sequence
  source: /note=“Description of Artificial Sequence: Synthetic peptide”
GGSIERYY

SEQ ID NO: 65 - protein, 7 aa, Artificial Sequence
  source: /note=“Description of Artificial Sequence: Synthetic peptide”
IYGRGST

SEQ ID NO: 66 - protein, 121 aa, Artificial Sequence
  source: /note=“Description of Artificial Sequence: Synthetic polypeptide”
QVQLQESGPGLVKPSETLSLTCTVSGGSIERYYWSWIRQPPGKGLEWIGYIYGRGSTNYNPSLKSRVTISVDTSKNQFSLKLSSVTAADTAVYYCARESQESPYNNWFDPWGQGTLVTVSS

SEQ ID NO: 67 - DNA, 363 bp, Artificial Sequence
  source: /note=“Description of Artificial Sequence: Synthetic polynucleotide”
caagtgcagctgcaggaatctggccctggcctggtcaagccctccgagacactgtccctgacctgcaccgtgtccggcggctccatcgagcggtactactggtcctggatccggcagcctcccggcaagggcctggaatggatcggctacatctacggcagaggctccaccaactacaaccccagcctgaagtccagagtgaccatctccgtggacacctccaagaaccagttctccctgaagctgtcctccgtgaccgccgctgacaccgccgtgtactactgcgccagagagtcccaggaatccccttacaacaattggttcgacccctggggccagggcaccctggtcaccgtgtcctct

SEQ ID NO: 68 - protein, 448 aa, Artificial Sequence
  source: /note=“Description of Artificial Sequence: Synthetic polypeptide”
  VARIANT (448)..(448): /replace=“ ”
  SITE (1)..(448): /note=“Variant residues given in the sequence have no preference with respect to those in the annotations for variant positions”
QVQLQESGPGLVKPSETLSLTCTVSGGSIERYYWSWIRQPPGKGLEWIGYIYGRGSTNYNPSLKSRVTISVDTSKNQFSLKLSSVTAADTAVYYCARESQESPYNNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 69 - DNA, 1344 bp, Artificial Sequence
  source: /note=“Description of Artificial Sequence: Synthetic polynucleotide”
  misc_feature (1342)..(1344): /note=“This region may or may not be present in its entirety”
  source: /note=“See specification as filed for detailed description of substitutions and preferred embodiments”
caagtgcagctgcaggaatctggccctggcctggtcaagccctccgagacactgtccctgacctgcaccgtgtccggcggctccatcgagcggtactactggtcctggatccggcagcctcccggcaagggcctggaatggatcggctacatctacggcagaggctccaccaactacaaccccagcctgaagtccagagtgaccatctccgtggacacctccaagaaccagttctccctgaagctgtcctccgtgaccgccgctgacaccgccgtgtactactgcgccagagagtcccaggaatccccttacaacaattggttcgacccctggggccagggcaccctggtcaccgtgtcctctgcctccaccaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgccctccttgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggcaag

SEQ ID NO: 70 - protein, 10 aa, Artificial Sequence
  source: /note=“Description of Artificial Sequence: Synthetic peptide”
GFTFSYNAMS

SEQ ID NO: 71 - protein, 17 aa, Artificial Sequence
  source: /note=“Description of Artificial Sequence: Synthetic peptide”
SISGTGGSTYYADSVKG

SEQ ID NO: 72 - protein, 5 aa, Artificial Sequence
  source: /note=“Description of Artificial Sequence: Synthetic peptide”
YNAMS

SEQ ID NO: 73 - protein, 7 aa, Artificial Sequence
  source: /note=“Description of Artificial Sequence: Synthetic peptide”
GFTFSYN

SEQ ID NO: 74 - protein, 6 aa, Artificial Sequence
  source: /note=“Description of Artificial Sequence: Synthetic peptide”
SGTGGS

SEQ ID NO: 75 - protein, 8 aa, Artificial Sequence
  source: /note=“Description of Artificial Sequence: Synthetic peptide”
GFTFSYNA

SEQ ID NO: 76 - protein, 8 aa, Artificial Sequence
  source: /note=“Description of Artificial Sequence: Synthetic peptide”
ISGTGGST

SEQ ID NO: 77 - protein, 124 aa, Artificial Sequence
  source: /note=“Description of Artificial Sequence: Synthetic polypeptide”
EVQLLESGGGLVQPGGSLRLSCAASGFTFSYNAMSWVRQAPGKGLEWVSSISGTGGSTYYADSVKGRFTISRDNSKNTLYLQMNSLRAEDTAVYYCARGGLYGSGSYLSDFDLWGRGTLVTVSS

SEQ ID NO: 78 - DNA, 372 bp, Artificial Sequence
  source: /note=“Description of Artificial Sequence: Synthetic polynucleotide”
gaagtgcagctgctggaatctggcggcggactggtgcagcctggcggctccctgagactgtcttgcgccgcctccggcttcaccttctcctacaacgccatgtcctgggtccgacaggcccctggcaagggcctggagtgggtgtcctccatctccggcaccggcggctccacctactacgccgactctgtgaagggccggttcaccatctcccgggacaactccaagaacaccctgtacctgcagatgaactccctgcgggccgaggacaccgccgtgtactactgtgccagaggcggcctgtacggctccggctcctacctgtccgacttcgacctgtggggcagaggcaccctggtcaccgtgtcctcc

SEQ ID NO: 79 - protein, 451 aa, Artificial Sequence
  source: /note=“Description of Artificial Sequence: Synthetic polypeptide”
  VARIANT (451)..(451): /replace=“ ”
  SITE (1)..(451): /note=“Variant residues given in the sequence have no preference with respect to those in the annotations for variant positions”
EVQLLESGGGLVQPGGSLRLSCAASGFTFSYNAMSWVRQAPGKGLEWVSSISGTGGSTYYADSVKGRFTISRDNSKNTLYLQMNSLRAEDTAVYYCARGGLYGSGSYLSDFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 80 - DNA, 1353 bp, Artificial Sequence
  source: /note=“Description of Artificial Sequence: Synthetic polynucleotide”
  misc_feature (1351)..(1353): /note=“This region may or may not be present in its entirety”
  source: /note=“See specification as filed for detailed description of substitutions and preferred embodiments”
gaagtgcagctgctggaatctggcggcggactggtgcagcctggcggctccctgagactgtcttgcgccgcctccggcttcaccttctcctacaacgccatgtcctgggtccgacaggcccctggcaagggcctggagtgggtgtcctccatctccggcaccggcggctccacctactacgccgactctgtgaagggccggttcaccatctcccgggacaactccaagaacaccctgtacctgcagatgaactccctgcgggccgaggacaccgccgtgtactactgtgccagaggcggcctgtacggctccggctcctacctgtccgacttcgacctgtggggcagaggcaccctggtcaccgtgtcctccgcctccacaaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgccctccttgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggcaag

SEQ ID NO: 81 - protein, 10 aa, Artificial Sequence
  source: /note=“Description of Artificial Sequence: Synthetic peptide”
GFTFSRYAMS

SEQ ID NO: 82 - protein, 7 aa, Artificial Sequence
  source: /note=“Description of Artificial Sequence: Synthetic peptide”
GFTFSRY

SEQ ID NO: 83 - protein, 8 aa, Artificial Sequence
  source: /note=“Description of Artificial Sequence: Synthetic peptide”
GFTFSRYA

SEQ ID NO: 84 - protein, 124 aa, Artificial Sequence
  source: /note=“Description of Artificial Sequence: Synthetic polypeptide”
EVQLLESGGGLVQPGGSLRLSCAASGFTFSRYAMSWVRQAPGKGLEWVSSISGTGGSTYYADSVKGRFTISRDNSKNTLYLQMNSLRAEDTAVYYCARGGLYGSGSYLSDFDLWGRGTLVTVSS

SEQ ID NO: 85 - DNA, 372 bp, Artificial Sequence
  source: /note=“Description of Artificial Sequence: Synthetic polynucleotide”
gaagtgcagctgctggaatctggcggcggactggtgcagcctggcggctccctgagactgtcttgcgccgcctccggcttcaccttctccagatacgccatgtcctgggtccgacaggcccctggcaagggcctggagtgggtgtcctccatctccggcaccggcggctccacctactacgccgactctgtgaagggccggttcaccatctcccgggacaactccaagaacaccctgtacctgcagatgaactccctgcgggccgaggacaccgccgtgtactactgtgccagaggcggcctgtacggctccggctcctacctgtccgacttcgacctgtggggcagaggcaccctggtcaccgtgtcctcc

SEQ ID NO: 86 - protein, 451 aa, Artificial Sequence
  source: /note=“Description of Artificial Sequence: Synthetic polypeptide”
  VARIANT (451)..(451): /replace=“ ”
  SITE (1)..(451): /note=“Variant residues given in the sequence have no preference with respect to those in the annotations for variant positions”
EVQLLESGGGLVQPGGSLRLSCAASGFTFSRYAMSWVRQAPGKGLEWVSSISGTGGSTYYADSVKGRFTISRDNSKNTLYLQMNSLRAEDTAVYYCARGGLYGSGSYLSDFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 87 - DNA, 1353 bp, Artificial Sequence
  source: /note=“Description of Artificial Sequence: Synthetic polynucleotide”
  misc_feature (1351)..(1353): /note=“This region may or may not be present in its entirety”
  source: /note=“See specification as filed for detailed description of substitutions and preferred embodiments”
gaagtgcagctgctggaatctggcggcggactggtgcagcctggcggctccctgagactgtcttgcgccgcctccggcttcaccttctccagatacgccatgtcctgggtccgacaggcccctggcaagggcctggagtgggtgtcctccatctccggcaccggcggctccacctactacgccgactctgtgaagggccggttcaccatctcccgggacaactccaagaacaccctgtacctgcagatgaactccctgcgggccgaggacaccgccgtgtactactgtgccagaggcggcctgtacggctccggctcctacctgtccgacttcgacctgtggggcagaggcaccctggtcaccgtgtcctccgcctccacaaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgccctccttgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggcaag

SEQ ID NO: 88 - protein, 5 aa, Artificial Sequence
  source: /note=“Description of Artificial Sequence: Synthetic peptide”
  VARIANT (1)..(1): /replace=“Tyr” or “Ser”
  VARIANT (2)..(2): /replace=“Asn”
  SITE (1)..(5): /note=“Variant residues given in the sequence have no preference with respect to those in the annotations for variant positions”
RYAMS

SEQ ID NO: 89 - protein, 17 aa, Artificial Sequence
  source: /note=“Description of Artificial Sequence: Synthetic peptide”
  VARIANT (1)..(1): /replace=“Ser”
  VARIANT (3)..(3): /replace=“Thr”
  VARIANT (5)..(5): /replace=“Thr”
  VARIANT (7)..(7): /replace=“Gly” or “Ser”
  VARIANT (8)..(8): /replace=“Ser” or “Leu” or “Tyr”
  SITE (1)..(17): /note=“Variant residues given in the sequence have no preference with respect to those in the annotations for variant positions”
AISGSGMNTYYADSVKG

SEQ ID NO: 90 - protein, 5 aa, Artificial Sequence
  source: /note=“Description of Artificial Sequence: Synthetic peptide”
  VARIANT (1)..(1): /replace=“Gly” or “Ser”
  VARIANT (2)..(2): /replace=“Arg”
  SITE (1)..(5): /note=“Variant residues given in the sequence have no preference with respect to those in the annotations for variant positions”
RYYWS

SEQ ID NO: 91 - protein, 16 aa, Artificial Sequence
  source: /note=“Description of Artificial Sequence: Synthetic peptide”
  VARIANT (4)..(4): /replace=“Ser”
  VARIANT (5)..(5): /replace=“Ser” or “Thr”
  VARIANT (9)..(9): /replace=“Lys”
  SITE (1)..(16): /note=“Variant residues given in the sequence have no preference with respect to those in the annotations for variant positions”
YIYGRGSTNYNPSLKS

SEQ ID NO: 92 - protein, 327 aa, Artificial Sequence
  source: /note=“Description of Artificial Sequence: Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 93 - protein, 326 aa, Artificial Sequence
  source: /note=“Description of Artificial Sequence: Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 94 - protein, 327 aa, Artificial Sequence
  source: /note=“Description of Artificial Sequence: Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 95 - protein, 326 aa, Artificial Sequence
  source: /note=“Description of Artificial Sequence: Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 96 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 98 - protein, 330 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 329 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 100 - protein, 330 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 329 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 330 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 329 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 104 - protein, 107 aa, Homo sapiens [sequence omitted]

SEQ ID NO: 105 - protein, 574 aa, Homo sapiens [sequence omitted]

SEQ ID NO: 106 - protein, 574 aa, Homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ

SEQ ID NO: 107 - protein, 524 aa, Homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ

SEQ ID NO: 108 - protein, 15 aa, Homo sapiens
YLPYKVLPVGDEVVG

SEQ ID NO: 109 - protein, 10 aa, Homo sapiens
KLKTLNVNKI

SEQ ID NO: 110 - protein, 20 aa, Homo sapiens
MINNNLRHADETFWNHVSMC

SEQ ID NO: 111 - protein, 18 aa, Homo sapiens
YQGTIWFTVYKGAEVAHF

SEQ ID NO: 112 - protein, 448 aa, Artificial Sequence
  source: /note=“Description of Artificial Sequence: Synthetic polypeptide”
  VARIANT (448)..(448): /replace=“ ”
  SITE (1)..(448): /note=“Variant residues given in the sequence have no preference with respect to those in the annotations for variant positions”
QVQLQESGPGLVKPSETLSLTCTVSGGSISGRYWSWIRQPPGKGLEWIGYIYGTGSTNYNPSLKSRVTISVDTSKNQFSLKLSSVTAADTAVYYCARESQESPYNNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 113 - protein, 448 aa, Artificial Sequence
  source: /note=“Description of Artificial Sequence: Synthetic polypeptide”
  VARIANT (448)..(448): /replace=“ ”
  SITE (1)..(448): /note=“Variant residues given in the sequence have no preference with respect to those in the annotations for variant positions”
QVQLQESGPGLVKPSETLSLTCTVSGGSIEGRYWSWIRQPPGKGLEWIGYIYGSGSTKYNPSLKSRVTISVDTSKNQFSLKLSSVTAADTAVYYCARESQESPYNNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 114 - protein, 451 aa, Artificial Sequence
  source: /note=“Description of Artificial Sequence: Synthetic polypeptide”
  VARIANT (451)..(451): /replace=“ ”
  SITE (1)..(451): /note=“Variant residues given in the sequence have no preference with respect to those in the annotations for variant positions”
EVQLLESGGGLVQPGGSLRLSCAASGFTFHRYAMSWVRQAPGKGLEWVSAISGSGMNTYYADSVKGRFTISRDNSKNTLYLQMNSLRAEDTAVYYCARGGLYGSGSYLSDFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 115 - protein, 448 aa, Artificial Sequence
  source: /note=“Description of Artificial Sequence: Synthetic polypeptide”
  VARIANT (448)..(448): /replace=“ ”
  SITE (1)..(448): /note=“Variant residues given in the sequence have no preference with respect to those in the annotations for variant positions”
QVQLQESGPGLVKPSETLSLTCTVSGGSIERYYWSWIRQPPGKGLEWIGYIYGRGSTNYNPSLKSRVTISVDTSKNQFSLKLSSVTAADTAVYYCARESQESPYNNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 116 - protein, 451 aa, Artificial Sequence
  source: /note=“Description of Artificial Sequence: Synthetic polypeptide”
  VARIANT (451)..(451): /replace=“ ”
  SITE (1)..(451): /note=“Variant residues given in the sequence have no preference with respect to those in the annotations for variant positions”
EVQLLESGGGLVQPGGSLRLSCAASGFTFSYNAMSWVRQAPGKGLEWVSSISGTGGSTYYADSVKGRFTISRDNSKNTLYLQMNSLRAEDTAVYYCARGGLYGSGSYLSDFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 117 - protein, 451 aa, Artificial Sequence
  source: /note=“Description of Artificial Sequence: Synthetic polypeptide”
  VARIANT (451)..(451): /replace=“ ”
  SITE (1)..(451): /note=“Variant residues given in the sequence have no preference with respect to those in the annotations for variant positions”
EVQLLESGGGLVQPGGSLRLSCAASGFTFSRYAMSWVRQAPGKGLEWVSSISGTGGSTYYADSVKGRFTISRDNSKNTLYLQMNSLRAEDTAVYYCARGGLYGSGSYLSDFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 118 - protein, 13 aa, Homo sapiens
RGNVISSHGNPIL

SEQ ID NO: 119 - protein, 330 aa, Homo sapiens [sequence omitted]

SEQ ID NO: 120 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 20 aa, Homo sapiens
MINNNLRHTDEMFWNHVSMC

SEQ ID NO: 122 - protein, 7 aa, Artificial Sequence
  source: /note=“Description of Artificial Sequence: Synthetic peptide”
  VARIANT (5)..(5): /replace=“Ser” or “Arg” or “Lys”
  VARIANT (6)..(6): /replace=“Tyr” or “Ser”
  VARIANT (7)..(7): /replace=“Asn”
  SITE (1)..(7): /note=“Variant residues given in the sequence have no preference with respect to those in the annotations for variant positions”
GFTFHRY

SEQ ID NO: 123 - protein, 6 aa, Artificial Sequence
  source: /note=“Description of Artificial Sequence: Synthetic peptide”
  VARIANT (1)..(1): /replace=“Thr”
  VARIANT (3)..(3): /replace=“Thr”
  VARIANT (5)..(5): /replace=“Gly” or “Ser”
  VARIANT (6)..(6): /replace=“Ser” or “Leu” or “Tyr”
  SITE (1)..(6): /note=“Variant residues given in the sequence have no preference with respect to those in the annotations for variant positions”
SGSGMN

SEQ ID NO: 124 - protein, 7 aa, Artificial Sequence
  source: /note=“Description of Artificial Sequence: Synthetic peptide”
  VARIANT (5)..(5): /replace=“Ser”
  VARIANT (6)..(6): /replace=“Gly” or “Ser”
  VARIANT (7)..(7): /replace=“Arg”
  SITE (1)..(7): /note=“Variant residues given in the sequence have no preference with respect to those in the annotations for variant positions”
GGSIERY

SEQ ID NO: 125 - protein, 5 aa, Artificial Sequence
  source: /note=“Description of Artificial Sequence: Synthetic peptide”
  VARIANT (2)..(2): /replace=“Ser”
  VARIANT (3)..(3): /replace=“Ser” or “Thr”
  SITE (1)..(5): /note=“Variant residues given in the sequence have no preference with respect to those in the annotations for variant positions”
YGRGS

SEQ ID NO: 126 - protein, 10 aa, Artificial Sequence
  source: /note=“Description of Artificial Sequence: Synthetic peptide”
  VARIANT (5)..(5): /replace=“Ser” or “Arg” or “Lys”
  VARIANT (6)..(6): /replace=“Tyr” or “Ser”
  VARIANT (7)..(7): /replace=“Asn”
  SITE (1)..(10): /note=“Variant residues given in the sequence have no preference with respect to those in the annotations for variant positions”
GFTFHRYAMS

SEQ ID NO: 127 - protein, 10 aa, Artificial Sequence
  source: /note=“Description of Artificial Sequence: Synthetic peptide”
  VARIANT (5)..(5): /replace=“Ser”
  VARIANT (6)..(6): /replace=“Gly” or “Ser”
  VARIANT (7)..(7): /replace=“Arg”
  SITE (1)..(10): /note=“Variant residues given in the sequence have no preference with respect to those in the annotations for variant positions”
GGSIERYYWS

SEQ ID NO: 128 - protein, 8 aa, Artificial Sequence
  source: /note=“Description of Artificial Sequence: Synthetic peptide”
  VARIANT (5)..(5): /replace=“Ser” or “Arg” or “Lys”
  VARIANT (6)..(6): /replace=“Tyr” or “Ser”
  VARIANT (7)..(7): /replace=“Asn”
  SITE (1)..(8): /note=“Variant residues given in the sequence have no preference with respect to those in the annotations for variant positions”
GFTFHRYA

SEQ ID NO: 129 - protein, 8 aa, Artificial Sequence
  source: /note=“Description of Artificial Sequence: Synthetic peptide”
  VARIANT (2)..(2): /replace=“Thr”
  VARIANT (4)..(4): /replace=“Thr”
  VARIANT (6)..(6): /replace=“Gly” or “Ser”
  VARIANT (7)..(7): /replace=“Ser” or “Leu” or “Tyr”
  SITE (1)..(8): /note=“Variant residues given in the sequence have no preference with respect to those in the annotations for variant positions”
ISGSGMNT

SEQ ID NO: 130 - protein, 8 aa, Artificial Sequence
  source: /note=“Description of Artificial Sequence: Synthetic peptide”
  VARIANT (5)..(5): /replace=“Ser”
  VARIANT (6)..(6): /replace=“Gly” or “Ser”
  VARIANT (7)..(7): /replace=“Arg”
  SITE (1)..(8): /note=“Variant residues given in the sequence have no preference with respect to those in the annotations for variant positions”
GGSIERYY

SEQ ID NO: 131 - protein, 7 aa, Artificial Sequence
  source: /note=“Description of Artificial Sequence: Synthetic peptide”
  VARIANT (3)..(3): /replace=“Ser”
  VARIANT (4)..(4): /replace=“Ser” or “Thr”
  SITE (1)..(7): /note=“Variant residues given in the sequence have no preference with respect to those in the annotations for variant positions”
IYGRGST

SEQ ID NO: 132 - protein, 486 aa, Artificial Sequence
  source: /note=“Description of Artificial Sequence: Synthetic polypeptide”
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 465 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 521 aa, Homo sapiens
WELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHADETFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIK

SEQ ID NO: 135 - protein, 10 aa, Artificial Sequence
  source: /note=“Description of Artificial Sequence: Synthetic peptide”
GFTFRSYAMS

SEQ ID NO: 136 - protein, 17 aa, Artificial Sequence
  source: /note=“Description of Artificial Sequence: Synthetic peptide”
AITGSGGLTYYADSVKG

SEQ ID NO: 137 - protein, 5 aa, Artificial Sequence
  source: /note=“Description of Artificial Sequence: Synthetic peptide”
SYAMS

SEQ ID NO: 138 - protein, 7 aa, Artificial Sequence
  source: /note=“Description of Artificial Sequence: Synthetic peptide”
GFTFRSY

SEQ ID NO: 139 - protein, 6 aa, Artificial Sequence
  source: /note=“Description of Artificial Sequence: Synthetic peptide”
TGSGGL

SEQ ID NO: 140 - protein, 8 aa, Artificial Sequence
  source: /note=“Description of Artificial Sequence: Synthetic peptide”
GFTFRSYA

SEQ ID NO: 141 - protein, 8 aa, Artificial Sequence
  source: /note=“Description of Artificial Sequence: Synthetic peptide”
ITGSGGLT

SEQ ID NO: 142 - protein, 124 aa, Artificial Sequence
  source: /note=“Description of Artificial Sequence: Synthetic polypeptide”
EVQLLESGGGLVQPGGSLRLSCAASGFTFRSYAMSWVRQAPGKGLEWVSAITGSGGLTYYADSVKGRFTISRDNSKNTLYLQMNSLRAEDTAVYYCARGGLYGSGSYLSDFDLWGRGTLVTVSS

SEQ ID NO: 143 - DNA, 372 bp, Artificial Sequence
  source: /note=“Description of Artificial Sequence: Synthetic polynucleotide”
gaggtgcagctgttggagtctgggggaggcttggtacagcctggggggtccctgagactctcctgtgcagcctctggattcacctttcgtagctatgccatgagctgggtccgccaggctccagggaaggggctggagtgggtctcagctattacgggaagtggtggtttgacatactacgcagactccgtgaagggccggttcaccatctccagagacaattccaagaacacgctgtatctgcaaatgaacagcctgagagccgaggacacggcggtgtactactgcgccagaggtggattgtacggaagcggaagctacttgagtgacttcgacctatgggggagaggtaccttggtcaccgtctcctca

SEQ ID NO: 144 - DNA, 321 bp, Artificial Sequence
  source: /note=“Description of Artificial Sequence: Synthetic polynucleotide”
gaaatagtgttgacgcagtctccagccaccctgtctgtgtctccaggggaaagagccaccctctcctgcagggccagtcagagtgttggcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagcacaatgccttcccttacacttttggcggagggaccaaggttgagatcaaa

SEQ ID NO: 145 - protein, 10 aa, Artificial Sequence
  source: /note=“Description of Artificial Sequence: Synthetic peptide”
GFTFKSYAMS

SEQ ID NO: 146 - protein, 17 aa, Artificial Sequence
  source: /note=“Description of Artificial Sequence: Synthetic peptide”
AISGSGSYTYYADSVKG

SEQ ID NO: 147 - protein, 7 aa, Artificial Sequence
  source: /note=“Description of Artificial Sequence: Synthetic peptide”
GFTFKSY

SEQ ID NO: 148 - protein, 6 aa, Artificial Sequence
  source: /note=“Description of Artificial Sequence: Synthetic peptide”
SGSGSY

SEQ ID NO: 149 - protein, 8 aa, Artificial Sequence
  source: /note=“Description of Artificial Sequence: Synthetic peptide”
GFTFKSYA

SEQ ID NO: 150 - protein, 8 aa, Artificial Sequence
  source: /note=“Description of Artificial Sequence: Synthetic peptide”
ISGSGSYT

SEQ ID NO: 151 - protein, 124 aa, Artificial Sequence
  source: /note=“Description of Artificial Sequence: Synthetic polypeptide”
EVQLLESGGGLVQPGGSLRLSCAASGFTFKSYAMSWVRQAPGKGLEWVSAISGSGSYTYYADSVKGRFTISRDNSKNTLYLQMNSLRAEDTAVYYCARGGLYGSGSYLSDFDLWGRGTLVTVSS

SEQ ID NO: 152 - DNA, 372 bp, Artificial Sequence
  source: /note=“Description of Artificial Sequence: Synthetic polynucleotide”
gaggtgcagctgttggagtctgggggaggcttggtacagcctggggggtccctgagactctcctgtgcagcctctggattcacctttaagagctatgccatgagttgggtccgccaggctccagggaaggggctggagtgggtctcagctattagtggaagtggttcgtatacatactacgcagactccgtgaagggccggttcaccatctccagagacaattccaagaacacgctgtatctgcaaatgaacagcctgagagccgaggacacggcggtgtactactgcgccagaggtggattgtacggaagcggaagctacttgagtgacttcgacctatgggggagaggtaccttggtcaccgtctcctca

SEQ ID NO: 153 - protein, 10 aa, Artificial Sequence
  source: /note=“Description of Artificial Sequence: Synthetic peptide”
GFTFSSYAMS

SEQ ID NO: 154 - protein, 17 aa, Artificial Sequence
  source: /note=“Description of Artificial Sequence: Synthetic peptide”
AISGSGGSTYYADSVKG

SEQ ID NO: 155 - protein, 7 aa, Artificial Sequence
  source: /note=“Description of Artificial Sequence: Synthetic peptide”
GFTFSSY

SEQ ID NO: 156 - protein, 6 aa, Artificial Sequence
  source: /note=“Description of Artificial Sequence: Synthetic peptide”
SGSGGS

SEQ ID NO: 157 - protein, 8 aa, Artificial Sequence
  source: /note=“Description of Artificial Sequence: Synthetic peptide”
GFTFSSYA

SEQ ID NO: 158 - protein, 8 aa, Artificial Sequence
  source: /note=“Description of Artificial Sequence: Synthetic peptide”
ISGSGGST

SEQ ID NO: 159 - protein, 124 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGGLYGSGSYLSDFDLWGRGTLVTVSS

SEQ ID NO: 160 - DNA, 372 bp, Artificial Sequence
  source: /note=“Description of Artificial Sequence: Synthetic polynucleotide”
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggcggtgtactactgcgccagaggtggattgtacggaagcggaagctacttgagtgacttcgacctatgggggagaggtaccttggtcaccgtctcctca

SEQ ID NO: 161 - protein, 10 aa, Artificial Sequence
  source: /note=“Description of Artificial Sequence: Synthetic peptide”
GGSISSYYWS

SEQ ID NO: 162 - protein, 16 aa, Artificial Sequence
  source: /note=“Description of Artificial Sequence: Synthetic peptide”
YIYSSGSTNYNPSLKS

SEQ ID NO: 163 - protein, 5 aa, Artificial Sequence
  source: /note=“Description of Artificial Sequence: Synthetic peptide”
SYYWS

SEQ ID NO: 164 - protein, 7 aa, Artificial Sequence
  source: /note=“Description of Artificial Sequence: Synthetic peptide”
GGSISSY

SEQ ID NO: 165 - protein, 5 aa, Artificial Sequence
  source: /note=“Description of Artificial Sequence: Synthetic peptide”
YSSGS

SEQ ID NO: 166 - protein, 8 aa, Artificial Sequence
  source: /note=“Description of Artificial Sequence: Synthetic peptide”
GGSISSYY

SEQ ID NO: 167 - protein, 7 aa, Artificial Sequence
  source: /note=“Description of Artificial Sequence: Synthetic peptide”
IYSSGST

SEQ ID NO: 168 - protein, 121 aa, Artificial Sequence
  source: /note=“Description of Artificial Sequence: Synthetic polypeptide”
QVQLQESGPGLVKPSETLSLTCTVSGGSISSYYWSWIRQPPGKGLEWIGYIYSSGSTNYNPSLKSRVTISVDTSKNQFSLKLSSVTAADTAVYYCARESQESPYNNWFDPWGQGTLVTVSS

SEQ ID NO: 169 - DNA, 363 bp, Artificial Sequence
  source: /note=“Description of Artificial Sequence: Synthetic polynucleotide”
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctatagtagtgggagcaccaactacaacccctccctcaagagtcgagtcaccatatcagtagacacgtccaagaaccagttctccctgaagctgagttctgtgaccgccgcagacacggcggtgtactactgcgccagagaatctcaggagagtccatacaacaattggttcgacccatggggacagggtacattggtcaccgtctcctca

SEQ ID NO: 170 - DNA, 321 bp, Artificial Sequence
  source: /note=“Description of Artificial Sequence: Synthetic polynucleotide”
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttattactgtcagcagggaaacagtttccctaggacttttggcggagggaccaaggttgagatcaaa

SEQ ID NO: 171 - protein, 527 aa, Artificial Sequence
  source: /note=“Description of Artificial Sequence: Synthetic polypeptide”
WELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHADETFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKHHHHHH

SEQ ID NO: 172 - protein, 447 aa, Artificial Sequence
  source: /note=“Description of Artificial Sequence: Synthetic polypeptide”
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 173 - protein, 215 aa, Artificial Sequence
  source: /note=“Description of Artificial Sequence: Synthetic polypeptide”
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - protein, 121 aa, Artificial Sequence
  source: /note=“Description of Artificial Sequence: Synthetic polypeptide”
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLWEVRALPSVYWGQGTLVTVSS

SEQ ID NO: 175 - protein, 109 aa, Artificial Sequence
  source: /note=“Description of Artificial Sequence: Synthetic polypeptide”
SYELTQPPSVSVAPGQTARITCGANDIGSKSVHWYQQKAGQAPVLVVSEDIIRPSGIPERISGSNSGNTATLTISRVEAGDEADYYCQVWDRDSDQYVFGTGTKVTVLG

SEQ ID NO: 176 - protein, 4 aa, Artificial Sequence
  source: /note=“Description of Artificial Sequence: Synthetic peptide”
RGDS

SEQ ID NO: 177 - protein, 449 aa, Artificial Sequence
  source: /note=“Description of Artificial Sequence: Synthetic polypeptide”
QVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 178 - protein, 485 aa, Artificial Sequence
  source: /note=“Description of Artificial Sequence: Synthetic polypeptide”
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 220 aa, Artificial Sequence
  source: /note=“Description of Artificial Sequence: Synthetic polypeptide”
DFVMTQSPDSLAVSLGERATINCKSSQSLLNTGNQKNYLTWYQQKPGQPPKLLIYWASTRESGVPDRFTGSGSGTDFTLTISSLQAEDVAVYYCQNDYSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - protein, 450 aa, Artificial Sequence
  source: /note=“Description of Artificial Sequence: Synthetic polypeptide”
QVQLVQSGAEVKKPGASVKVSCKASGYTFTSYNMHWVRQAPGQGLEWMGAIYPGNGDTSYNQKFQGRVTITADKSISTAYMELSSLRSEDTAVYYCARSTYYGGDWYFNVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 181 - protein, 485 aa, Artificial Sequence; identical to SEQ ID NO: 178
  source: /note=“Description of Artificial Sequence: Synthetic polypeptide”

SEQ ID NO: 182 - protein, 213 aa, Artificial Sequence
  source: /note=“Description of Artificial Sequence: Synthetic polypeptide”
QIVLTQSPSSLSASVGDRVTITCRASSSVSYIHWFQQKPGKSPKPLIYATSNLASGVPVRFSGSGSGTDYTLTISSLQPEDFATYYCQQWTSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 183 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 184 - protein, 170 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 185 - protein, 114 aa, Artificial Sequence
  source: /note=“Description of Artificial Sequence: Synthetic polypeptide”
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 186 - protein, 297 aa, Artificial Sequence
  source: /note=“Description of Artificial Sequence: Synthetic polypeptide”
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 114 aa, Homo sapiens; identical to SEQ ID NO: 183
  VARIANT (93)..(93): /replace=“Lys”
  SITE (1)..(114): /note=“Variant residues given in the sequence have no preference with respect to those in the annotations for variant positions”

SEQ ID NO: 188 - protein, 77 aa, Homo sapiens
ITCPPPMSVEHADIWVKSYSLYSRERYICNSGFKRKAGTSSLTECVLNKATNVAHWTTPSLKCIRDPALVHQRPAPP

SEQ ID NO: 189 - protein, 9 aa, Artificial Sequence
  source: /note=“Description of Artificial Sequence: Synthetic peptide”
  VARIANT (4)..(4): /replace=“Ser” or “Arg” or “Lys”
  VARIANT (5)..(5): /replace=“Tyr” or “Ser”
  VARIANT (6)..(6): /replace=“Asn”
  SITE (1)..(9): /note=“Variant residues given in the sequence have no preference with respect to those in the annotations for variant positions”
FTFHRYAMS

SEQ ID NO: 190 - protein, 9 aa, Artificial Sequence
  source: /note=“Description of Artificial Sequence: Synthetic peptide”
FTFHRYAMS

SEQ ID NO: 191 - protein, 9 aa, Artificial Sequence
  source: /note=“Description of Artificial Sequence: Synthetic peptide”
FTFSYNAMS

SEQ ID NO: 192 - protein, 9 aa, Artificial Sequence
  source: /note=“Description of Artificial Sequence: Synthetic peptide”
FTFSRYAMS

SEQ ID NO: 193 - protein, 9 aa, Artificial Sequence
  source: /note=“Description of Artificial Sequence: Synthetic peptide”
FTFRSYAMS

SEQ ID NO: 194 - protein, 9 aa, Artificial Sequence
  source: /note=“Description of Artificial Sequence: Synthetic peptide”
FTFKSYAMS

SEQ ID NO: 195 - protein, 9 aa, Artificial Sequence
  source: /note=“Description of Artificial Sequence: Synthetic peptide”
FTFSSYAMS

SEQ ID NO: 196 - protein, 9 aa, Artificial Sequence
  source: /note=“Description of Artificial Sequence: Synthetic peptide”
  VARIANT (4)..(4): /replace=“Ser”
  VARIANT (5)..(5): /replace=“Gly” or “Ser”
  VARIANT (6)..(6): /replace=“Arg”
  SITE (1)..(9): /note=“Variant residues given in the sequence have no preference with respect to those in the annotations for variant positions”
GSIERYYWS

SEQ ID NO: 197 - protein, 9 aa, Artificial Sequence
  source: /note=“Description of Artificial Sequence: Synthetic peptide”
GSIERYYWS

SEQ ID NO: 198 - protein, 9 aa, Artificial Sequence
  source: /note=“Description of Artificial Sequence: Synthetic peptide”
GSIEGRYWS

SEQ ID NO: 199 - protein, 9 aa, Artificial Sequence
  source: /note=“Description of Artificial Sequence: Synthetic peptide”
GSISGRYWS

SEQ ID NO: 200 - protein, 9 aa, Artificial Sequence
  source: /note=“Description of Artificial Sequence: Synthetic peptide”
GSISSYYWS

SEQ ID NO: 201-209 - skipped (9 entries)

SEQ ID NO: 210 - protein, 10 aa, Artificial Sequence
  source: /note=“Description of Artificial Sequence: Synthetic peptide”
GGSFSGYYWS

SEQ ID NO: 211 - protein, 16 aa, Artificial Sequence
  source: /note=“Description of Artificial Sequence: Synthetic peptide”
EIDHSGSTNYNPSLKS

SEQ ID NO: 212 - protein, 15 aa, Artificial Sequence
  source: /note=“Description of Artificial Sequence: Synthetic peptide”
EGGLGEDYYYYGMDV

SEQ ID NO: 213 - protein, 5 aa, Artificial Sequence
  source: /note=“Description of Artificial Sequence: Synthetic peptide”
GYYWS

SEQ ID NO: 214 - protein, 7 aa, Artificial Sequence
  source: /note=“Description of Artificial Sequence: Synthetic peptide”
GGSFSGY

SEQ ID NO: 215 - protein, 5 aa, Artificial Sequence
  source: /note=“Description of Artificial Sequence: Synthetic peptide”
DHSGS

SEQ ID NO: 216 - protein, 8 aa, Artificial Sequence
  source: /note=“Description of Artificial Sequence: Synthetic peptide”
GGSFSGYY

SEQ ID NO: 217 - protein, 7 aa, Artificial Sequence
  source: /note=“Description of Artificial Sequence: Synthetic peptide”
IDHSGST

SEQ ID NO: 218 - protein, 17 aa, Artificial Sequence
  source: /note=“Description of Artificial Sequence: Synthetic peptide”
AREGGLGEDYYYYGMDV

SEQ ID NO: 219 - protein, 9 aa, Artificial Sequence
  source: /note=“Description of Artificial Sequence: Synthetic peptide”
GSFSGYYWS

SEQ ID NO: 220 - protein, 123 aa, Artificial Sequence
  source: /note=“Description of Artificial Sequence: Synthetic polypeptide”
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCAREGGLGEDYYYYGMDVWGQGTTVTVSS

SEQ ID NO: 221 - protein, 11 aa, Artificial Sequence
  source: /note=“Description of Artificial Sequence: Synthetic peptide”
RASQSISSYLN

SEQ ID NO: 222 - protein, 7 aa, Artificial Sequence
  source: /note=“Description of Artificial Sequence: Synthetic peptide”
GASSLQS

SEQ ID NO: 223 - protein, 9 aa, Artificial Sequence
  source: /note=“Description of Artificial Sequence: Synthetic peptide”
QQQYPLPPT

SEQ ID NO: 224 - protein, 7 aa, Artificial Sequence
  source: /note=“Description of Artificial Sequence: Synthetic peptide”
SQSISSY

SEQ ID NO: 225 - protein, 6 aa, Artificial Sequence
  source: /note=“Description of Artificial Sequence: Synthetic peptide”
QYPLPP

SEQ ID NO: 226 - protein, 6 aa, Artificial Sequence
  source: /note=“Description of Artificial Sequence: Synthetic peptide”
QSISSY

SEQ ID NO: 227 - protein, 107 aa, Artificial Sequence
  source: /note=“Description of Artificial Sequence: Synthetic polypeptide”
DIQMTQSPSSLSASVGDRVTITCRASQSISSYLNWYQQKPGKAPKLLIYGASSLQSGVPSRFSGSGSGTDFTLTISSLQPEDFATYYCQQQYPLPPTFGGGTKVEIK

SEQ ID NO: 228-269 - skipped (42 entries)

SEQ ID NO: 270 - protein, 12 aa, Artificial Sequence
  source: /note=“Description of Artificial Sequence: Synthetic peptide”
GGSILSSSYYWV

SEQ ID NO: 271 - protein, 16 aa, Artificial Sequence
  source: /note=“Description of Artificial Sequence: Synthetic peptide”
SIYYSGGTDYNPSLKS

SEQ ID NO: 272 - protein, 10 aa, Artificial Sequence
  source: /note=“Description of Artificial Sequence: Synthetic peptide”
VMSGWLDLDV

SEQ ID NO: 273 - protein, 7 aa, Artificial Sequence
  source: /note=“Description of Artificial Sequence: Synthetic peptide”
SSSYYWV

SEQ ID NO: 274 - protein, 9 aa, Artificial Sequence
  source: /note=“Description of Artificial Sequence: Synthetic peptide”
GGSILSSSY

SEQ ID NO: 275 - protein, 5 aa, Artificial Sequence
  source: /note=“Description of Artificial Sequence: Synthetic peptide”
YYSGG

SEQ ID NO: 276 - protein, 10 aa, Artificial Sequence
  source: /note=“Description of Artificial Sequence: Synthetic peptide”
GGSILSSSYY

SEQ ID NO: 277 - protein, 7 aa, Artificial Sequence
  source: /note=“Description of Artificial Sequence: Synthetic peptide”
IYYSGGT

SEQ ID NO: 278 - protein, 12 aa, Artificial Sequence
  source: /note=“Description of Artificial Sequence: Synthetic peptide”
ARVMSGWLDLDV

SEQ ID NO: 279 - protein, 11 aa, Artificial Sequence
  source: /note=“Description of Artificial Sequence: Synthetic peptide”
GSILSSSYYWV

SEQ ID NO: 280 - protein, 120 aa, Artificial Sequence
  source: /note=“Description of Artificial Sequence: Synthetic polypeptide”
QLQLQESGPGLVKPSETLSLTCTVSGGSILSSSYYWVWIRQPPGKGLEWIGSIYYSGGTDYNPSLKSRVTISVDTSKNQFSLKLSSVTAADTAVYYCARVMSGWLDLDVWGQGTMVTVSS

SEQ ID NO: 281 - protein, 11 aa, Artificial Sequence
  source: /note=“Description of Artificial Sequence: Synthetic peptide”
RASQSINSYLN

SEQ ID NO: 282 - protein, 9 aa, Artificial Sequence
  source: /note=“Description of Artificial Sequence: Synthetic peptide”
QQAYARPRT

SEQ ID NO: 283 - protein, 7 aa, Artificial Sequence
  source: /note=“Description of Artificial Sequence: Synthetic peptide”
SQSINSY

SEQ ID NO: 284 - protein, 6 aa, Artificial Sequence
  source: /note=“Description of Artificial Sequence: Synthetic peptide”
AYARPR

SEQ ID NO: 285 - protein, 6 aa, Artificial Sequence
  source: /note=“Description of Artificial Sequence: Synthetic peptide”
QSINSY

SEQ ID NO: 286 - protein, 107 aa, Artificial Sequence
  source: /note=“Description of Artificial Sequence: Synthetic polypeptide”
DIQMTQSPSSLSASVGDRVTITCRASQSINSYLNWYQQKPGKAPKLLIYAASSLQSGVPSRFSGSGSGTDFTLTISSLQPEDFATYYCQQAYARPRTFGGGTKVEIK

SEQ ID NO: 287 - protein, 10 aa, Artificial Sequence
  source: /note=“Description of Artificial Sequence: Synthetic peptide”
GGTFSSYAIS

SEQ ID NO: 288 - protein, 17 aa, Artificial Sequence
  source: /note=“Description of Artificial Sequence: Synthetic peptide”
GIIPIFGTANYAQKFQG

SEQ ID NO: 289 - protein, 16 aa, Artificial Sequence
  source: /note=“Description of Artificial Sequence: Synthetic peptide”
HYYDYWSGYYTNTGIY

SEQ ID NO: 290 - protein, 5 aa, Artificial Sequence
  source: /note=“Description of Artificial Sequence: Synthetic peptide”
SYAIS

SEQ ID NO: 291 - protein, 7 aa, Artificial Sequence
  source: /note=“Description of Artificial Sequence: Synthetic peptide”
GGTFSSY

SEQ ID NO: 292 - protein, 6 aa, Artificial Sequence
  source: /note=“Description of Artificial Sequence: Synthetic peptide”
IPIFGT

SEQ ID NO: 293 - protein, 8 aa, Artificial Sequence
  source: /note=“Description of Artificial Sequence: Synthetic peptide”
GGTFSSYA

SEQ ID NO: 294 - protein, 8 aa, Artificial Sequence
  source: /note=“Description of Artificial Sequence: Synthetic peptide”
IIPIFGTA

SEQ ID NO: 295 - protein, 18 aa, Artificial Sequence
  source: /note=“Description of Artificial Sequence: Synthetic peptide”
ARHYYDYWSGYYTNTGIY

SEQ ID NO: 296 - protein, 9 aa, Artificial Sequence
  source: /note=“Description of Artificial Sequence: Synthetic peptide”
GTFSSYAIS

SEQ ID NO: 297 - protein, 125 aa, Artificial Sequence
  source: /note=“Description of Artificial Sequence: Synthetic polypeptide”
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARHYYDYWSGYYTNTGIYWGQGTLVTVSS

SEQ ID NO: 298 - protein, 11 aa, Artificial Sequence
  source: /note=“Description of Artificial Sequence: Synthetic peptide”
RASQSVSSNLA

SEQ ID NO: 299 - protein, 9 aa, Artificial Sequence
  source: /note=“Description of Artificial Sequence: Synthetic peptide”
QQSGALPIT

SEQ ID NO: 300 - protein, 7 aa, Artificial Sequence
  source: /note=“Description of Artificial Sequence: Synthetic peptide”
SQSVSSN

SEQ ID NO: 301 - protein, 6 aa, Artificial Sequence
  source: /note=“Description of Artificial Sequence: Synthetic peptide”
SGALPI

SEQ ID NO: 302 - protein, 6 aa, Artificial Sequence
  source: /note=“Description of Artificial Sequence: Synthetic peptide”
QSVSSN

SEQ ID NO: 303 - protein, 107 aa, Artificial Sequence
  source: /note=“Description of Artificial Sequence: Synthetic polypeptide”
EIVMTQSPATLSVSPGERATLSCRASQSVSSNLAWYQQKPGQAPRLLIYGASTRATGIPARFSGSGSGTEFTLTISSLQSEDFAVYYCQQSGALPITFGGGTKVEIK

SEQ ID NO: 304-332 - skipped (29 entries)

SEQ ID NO: 333 - protein, 10 aa, Artificial Sequence
  source: /note=“Description of Artificial Sequence: Synthetic peptide”
GGTFSSLAIS

SEQ ID NO: 334 - protein, 11 aa, Artificial Sequence
  source: /note=“Description of Artificial Sequence: Synthetic peptide”
EGAGWYYAFDI

SEQ ID NO: 335 - protein, 5 aa, Artificial Sequence
  source: /note=“Description of Artificial Sequence: Synthetic peptide”
SLAIS

SEQ ID NO: 336 - protein, 7 aa, Artificial Sequence
  source: /note=“Description of Artificial Sequence: Synthetic peptide”
GGTFSSL

SEQ ID NO: 337 - protein, 8 aa, Artificial Sequence
  source: /note=“Description of Artificial Sequence: Synthetic peptide”
GGTFSSLA

SEQ ID NO: 338 - protein, 13 aa, Artificial Sequence
  source: /note=“Description of Artificial Sequence: Synthetic peptide”
AREGAGWYYAFDI

SEQ ID NO: 339 - protein, 9 aa, Artificial Sequence
  source: /note=“Description of Artificial Sequence: Synthetic peptide”
GTFSSLAIS

SEQ ID NO: 340 - protein, 120 aa, Artificial Sequence
  source: /note=“Description of Artificial Sequence: Synthetic polypeptide”
QVQLVQSGAEVKKPGSSVKVSCKASGGTFSSLAISWVRQAPGQGLEWMGGIIPIFGTANYAQKFQGRVTITADESTSTAYMELSSLRSEDTAVYYCAREGAGWYYAFDIWGQGTMVTVSS

SEQ ID NO: 341 - protein, 9 aa, Artificial Sequence
  source: /note=“Description of Artificial Sequence: Synthetic peptide”
QQAYVKPIT

SEQ ID NO: 342 - protein, 6 aa, Artificial Sequence
  source: /note=“Description of Artificial Sequence: Synthetic peptide”
AYVKPI

SEQ ID NO: 343 - protein, 107 aa, Artificial Sequence
  source: /note=“Description of Artificial Sequence: Synthetic polypeptide”
DIQMTQSPSSLSASVGDRVTITCRASQSISSYLNWYQQKPGKAPKLLIYGASSLQSGVPSRFSGSGSGTDFTLTISSLQPEDFATYYCQQAYVKPITFGGGTKVEIK

SEQ ID NO: 344 - protein, 11 aa, Artificial Sequence
  source: /note=“Description of Artificial Sequence: Synthetic peptide”
  VARIANT (7)..(7): /replace=“Asn”
  SITE (1)..(11): /note=“Variant residues given in the sequence have no preference with respect to those in the annotations for variant positions”
RASQSISSYLN

SEQ ID NO: 345 - protein, 7 aa, Artificial Sequence
  source: /note=“Description of Artificial Sequence: Synthetic peptide”
  VARIANT (1)..(1): /replace=“Ala”
  SITE (1)..(7): /note=“Variant residues given in the sequence have no preference with respect to those in the annotations for variant positions”
GASSLQS

SEQ ID NO: 346 - protein, 9 aa, Artificial Sequence
  source: /note=“Description of Artificial Sequence: Synthetic peptide”
  VARIANT (3)..(3): /replace=“Ala”
  VARIANT (5)..(5): /replace=“Val” or “Ala”
  VARIANT (6)..(6): /replace=“Lys” or “Arg”
  VARIANT (8)..(8): /replace=“Ile” or “Arg”
  SITE (1)..(9): /note=“Variant residues given in the sequence have no preference with respect to those in the annotations for variant positions”
QQQYPLPPT

SEQ ID NO: 347 - protein, 5 aa, Artificial Sequence
  source: /note=“Description of Artificial Sequence: Synthetic peptide”
  VARIANT (2)..(2): /replace=“Tyr”
  SITE (1)..(5): /note=“Variant residues given in the sequence have no preference with respect to those in the annotations for variant positions”
SLAIS

SEQ ID NO: 348-500 - skipped (153 entries)

SEQ ID NO: 501 - protein, 5 aa, Artificial Sequence
  source: /note=“Description of Artificial Sequence: Synthetic peptide”
TYWMH

SEQ ID NO: 502 - protein, 17 aa, Artificial Sequence
  source: /note=“Description of Artificial Sequence: Synthetic peptide”
NIYPGTGGSNFDEKFKN

SEQ ID NO: 503 - protein, 8 aa, Artificial Sequence
  source: /note=“Description of Artificial Sequence: Synthetic peptide”
WTTGTGAY

SEQ ID NO: 504 - protein, 7 aa, Artificial Sequence
  source: /note=“Description of Artificial Sequence: Synthetic peptide”
GYTFTTY

SEQ ID NO: 505 - protein, 6 aa, Artificial Sequence
  source: /note=“Description of Artificial Sequence: Synthetic peptide”
YPGTGG

SEQ ID NO: 506 - protein, 117 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 507 - DNA, 351 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagc

SEQ ID NO: 508 - protein, 443 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 509 - DNA, 1329 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 510 - protein, 17 aa, Artificial Sequence
  source: /note=“Description of Artificial Sequence: Synthetic peptide”
KSSQSLLDSGNQKNFLT

SEQ ID NO: 511 - protein, 7 aa, Artificial Sequence
  source: /note=“Description of Artificial Sequence: Synthetic peptide”
WASTRES

SEQ ID NO: 512 - protein, 9 aa, Artificial Sequence
  source: /note=“Description of Artificial Sequence: Synthetic peptide”
QNDYSYPYT

SEQ ID NO: 513 - protein, 13 aa, Artificial Sequence
  source: /note=“Description of Artificial Sequence: Synthetic peptide”
SQSLLDSGNQKNF

SEQ ID NO: 514 - protein, 3 aa, Artificial Sequence
  source: /note=“Description of Artificial Sequence: Synthetic peptide”
WAS

SEQ ID NO: 515 - protein, 6 aa, Artificial Sequence
  source: /note=“Description of Artificial Sequence: Synthetic peptide”
DYSYPY

SEQ ID NO: 516 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 517 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaag

SEQ ID NO: 518 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 519 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 520 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 521 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaag

SEQ ID NO: 522 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 523 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 524 - DNA, 15 bp, Artificial Sequence
  source: /note=“Description of Artificial Sequence: Synthetic oligonucleotide”
acctactggatgcac

SEQ ID NO: 525 - DNA, 51 bp, Artificial Sequence
  source: /note=“Description of Artificial Sequence: Synthetic oligonucleotide”
aatatctaccccggcaccggcggctctaacttcgacgagaagtttaagaat

SEQ ID NO: 526 - DNA, 24 bp, Artificial Sequence
  source: /note=“Description of Artificial Sequence: Synthetic oligonucleotide”
tggactaccggcacaggcgcctac

SEQ ID NO: 527 - DNA, 21 bp, Artificial Sequence
  source: /note=“Description of Artificial Sequence: Synthetic oligonucleotide”
ggctacaccttcactacctac

SEQ ID NO: 528 - DNA, 18 bp, Artificial Sequence
  source: /note=“Description of Artificial Sequence: Synthetic oligonucleotide”
taccccggcaccggcggc

SEQ ID NO: 529 - DNA, 51 bp, Artificial Sequence
  source: /note=“Description of Artificial Sequence: Synthetic oligonucleotide”
aaatctagtcagtcactgctggatagcggtaatcagaagaacttcctgacc

SEQ ID NO: 530 - DNA, 21 bp, Artificial Sequence
  source: /note=“Description of Artificial Sequence: Synthetic oligonucleotide”
tgggcctctactagagaatca

SEQ ID NO: 531 - DNA, 27 bp, Artificial Sequence
  source: /note=“Description of Artificial Sequence: Synthetic oligonucleotide”
cagaacgactatagctacccctacacc

SEQ ID NO: 532 - DNA, 39 bp, Artificial Sequence
  source: /note=“Description of Artificial Sequence: Synthetic oligonucleotide”
agtcagtcactgctggatagcggtaatcagaagaacttc

SEQ ID NO: 533 - DNA, 9 bp, Artificial Sequence
  source: /note=“Description of Artificial Sequence: Synthetic oligonucleotide”
tgggcctct

SEQ ID NO: 534 - DNA, 18 bp, Artificial Sequence
  source: /note=“Description of Artificial Sequence: Synthetic oligonucleotide”
gactatagctacccctac

SEQ ID NO: 535 - protein, 440 aa, Artificial Sequence
  source: /note=“Description of Artificial Sequence: Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 536 - protein, 214 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 537 - protein, 447 aa, Artificial Sequence
  source: /note=“Description of Artificial Sequence: Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 538 - protein, 218 aa, Artificial Sequence
  source: /note=“Description of Artificial Sequence: Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 539 - protein, 447 aa, Artificial Sequence
  source: /note=“Description of Artificial Sequence: Synthetic polypeptide”
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 540 - protein, 213 aa, Artificial Sequence
  source: /note=“Description of Artificial Sequence: Synthetic polypeptide”
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 541 - protein, 10 aa, Artificial Sequence
  source: /note=“Description of Artificial Sequence: Synthetic peptide”
GYTFTTYWMH

SEQ ID NO: 542-600 - skipped (59 entries)

SEQ ID NO: 601 - protein, 5 aa, Artificial Sequence
  source: /note=“Description of Artificial Sequence: Synthetic peptide”
SYWMY

SEQ ID NO: 602 - protein, 17 aa, Artificial Sequence
  source: /note=“Description of Artificial Sequence: Synthetic peptide”
RIDPNSGSTKYNEKFKN

SEQ ID NO: 603 - protein, 11 aa, Artificial Sequence
  source: /note=“Description of Artificial Sequence: Synthetic peptide”
DYRKGLYAMDY

SEQ ID NO: 604 - protein, 7 aa, Artificial Sequence
  source: /note=“Description of Artificial Sequence: Synthetic peptide”
GYTFTSY

SEQ ID NO: 605 - protein, 6 aa, Artificial Sequence
  source: /note=“Description of Artificial Sequence: Synthetic peptide”
DPNSGS

SEQ ID NO: 606 - protein, 120 aa, Artificial Sequence
  source: /note=“Description of Artificial Sequence: Synthetic polypeptide”
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSS

SEQ ID NO: 607 - DNA, 360 bp, Artificial Sequence
  source: /note=“Description of Artificial Sequence: Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttca

SEQ ID NO: 608 - protein, 446 aa, Artificial Sequence
  source: /note=“Description of Artificial Sequence: Synthetic polypeptide”
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 609 - protein, 11 aa, Artificial Sequence
  source: /note=“Description of Artificial Sequence: Synthetic peptide”
KASQDVGTAVA

SEQ ID NO: 610 - protein, 7 aa, Artificial Sequence
  source: /note=“Description of Artificial Sequence: Synthetic peptide”
WASTRHT

SEQ ID NO: 611 - protein, 9 aa, Artificial Sequence
  source: /note=“Description of Artificial Sequence: Synthetic peptide”
QQYNSYPLT

SEQ ID NO: 612 - protein, 7 aa, Artificial Sequence
  source: /note=“Description of Artificial Sequence: Synthetic peptide”
SQDVGTA

SEQ ID NO: 613 - protein, 3 aa, Artificial Sequence
  source: /note=“Description of Artificial Sequence: Synthetic peptide”
WAS

SEQ ID NO: 614 - protein, 6 aa, Artificial Sequence
  source: /note=“Description of Artificial Sequence: Synthetic peptide”
YNSYPL

SEQ ID NO: 615 - DNA, 1338 bp, Artificial Sequence
  source: /note=“Description of Artificial Sequence: Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 616 - protein, 107 aa, Artificial Sequence
  source: /note=“Description of Artificial Sequence: Synthetic polypeptide”
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIK

SEQ ID NO: 617 - DNA, 321 bp, Artificial Sequence
  source: /note=“Description of Artificial Sequence: Synthetic polynucleotide”
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaag

SEQ ID NO: 618 - protein, 214 aa, Artificial Sequence
  source: /note=“Description of Artificial Sequence: Synthetic polypeptide”
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 619 - DNA, 642 bp, Artificial Sequence
  source: /note=“Description of Artificial Sequence: Synthetic polynucleotide”
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 620 - protein, 120 aa, Artificial Sequence
  source: /note=“Description of Artificial Sequence: Synthetic polypeptide”
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSS

SEQ ID NO: 621 - DNA, 360 bp, Artificial Sequence
  source: /note=“Description of Artificial Sequence: Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttca

SEQ ID NO: 622 - protein, 446 aa, Artificial Sequence
  source: /note=“Description of Artificial Sequence: Synthetic polypeptide”
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 623 - DNA, 1338 bp, Artificial Sequence
  source: /note=“Description of Artificial Sequence: Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 624 - protein, 107 aa, Artificial Sequence
  source: /note=“Description of Artificial Sequence: Synthetic polypeptide”
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIK

SEQ ID NO: 625 - DNA, 321 bp, Artificial Sequence
  source: /note=“Description of Artificial Sequence: Synthetic polynucleotide”
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaag

SEQ ID NO: 626 - protein, 214 aa, Artificial Sequence
  source: /note=“Description of Artificial Sequence: Synthetic polypeptide”
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 627 - DNA, 642 bp, Artificial Sequence
  source: /note=“Description of Artificial Sequence: Synthetic polynucleotide”
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 628 - DNA, 15 bp, Artificial Sequence
  source: /note=“Description of Artificial Sequence: Synthetic oligonucleotide”
agctactggatgtac

SEQ ID NO: 629 - DNA, 51 bp, Artificial Sequence
  source: /note=“Description of Artificial Sequence: Synthetic oligonucleotide”
agaatcgaccctaatagcggctctactaagtataacgagaagtttaagaat

SEQ ID NO: 630 - DNA, 33 bp, Artificial Sequence
  source: /note=“Description of Artificial Sequence: Synthetic oligonucleotide”
gactatagaaagggcctgtacgctatggactac

SEQ ID NO: 631 - DNA, 21 bp, Artificial Sequence
  source: /note=“Description of Artificial Sequence: Synthetic oligonucleotide”
ggctacaccttcactagctac

SEQ ID NO: 632 - DNA, 18 bp, Artificial Sequence
  source: /note=“Description of Artificial Sequence: Synthetic oligonucleotide”
gaccctaatagcggctct

SEQ ID NO: 633 - DNA, 33 bp, Artificial Sequence
  source: /note=“Description of Artificial Sequence: Synthetic oligonucleotide”
aaagcctctcaggacgtgggcaccgccgtggcc

SEQ ID NO: 634 - DNA, 21 bp, Artificial Sequence
  source: /note=“Description of Artificial Sequence: Synthetic oligonucleotide”
tgggcctctactagacacacc

SEQ ID NO: 635 - DNA, 27 bp, Artificial Sequence
  source: /note=“Description of Artificial Sequence: Synthetic oligonucleotide”
cagcagtataatagctaccccctgacc

SEQ ID NO: 636 - DNA, 21 bp, Artificial Sequence
  source: /note=“Description of Artificial Sequence: Synthetic oligonucleotide”
tctcaggacgtgggcaccgcc

SEQ ID NO: 637 - DNA, 9 bp, Artificial Sequence
  source: /note=“Description of Artificial Sequence: Synthetic oligonucleotide”
tgggcctct

SEQ ID NO: 638 - DNA, 18 bp, Artificial Sequence
  source: /note=“Description of Artificial Sequence: Synthetic oligonucleotide”
tataatagctaccccctg

SEQ ID NO: 639 - protein, 448 aa, Artificial Sequence
  source: /note=“Description of Artificial Sequence: Synthetic polypeptide”
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 640 - protein, 214 aa, Artificial Sequence
  source: /note=“Description of Artificial Sequence: Synthetic polypeptide”
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 641 - protein, 450 aa, Artificial Sequence
  source: /note=“Description of Artificial Sequence: Synthetic polypeptide”
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 642 - protein, 216 aa, Artificial Sequence
  source: /note=“Description of Artificial Sequence: Synthetic polypeptide”
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 643 - protein, 451 aa, Artificial Sequence
  source: /note=“Description of Artificial Sequence: Synthetic polypeptide”
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 644 - protein, 215 aa, Artificial Sequence
  source: /note=“Description of Artificial Sequence: Synthetic polypeptide”
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 645 - protein, 123 aa, Artificial Sequence
  source: /note=“Description of Artificial Sequence: Synthetic polypeptide”
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 646 - protein, 106 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 647 - protein, 10 aa, Artificial Sequence
  source: /note=“Description of Artificial Sequence: Synthetic peptide”
GYTFTSYWMY

SEQ ID NO: 648-700 - skipped (53 entries)

SEQ ID NO: 701 - protein, 5 aa, Artificial Sequence
  source: /note=“Description of Artificial Sequence: Synthetic peptide”
NYGMN

SEQ ID NO: 702 - protein, 17 aa, Artificial Sequence
  source: /note=“Description of Artificial Sequence: Synthetic peptide”
WINTDTGEPTYADDFKG

SEQ ID NO: 703 - protein, 16 aa, Artificial Sequence
  source: /note=“Description of Artificial Sequence: Synthetic peptide”
NPPYYYGTNNAEAMDY

SEQ ID NO: 704 - protein, 7 aa, Artificial Sequence
  source: /note=“Description of Artificial Sequence: Synthetic peptide”
GFTLTNY

SEQ ID NO: 705 - protein, 6 aa, Artificial Sequence
  source: /note=“Description of Artificial Sequence: Synthetic peptide”
NTDTGE

SEQ ID NO: 706 - protein, 125 aa, Artificial Sequence
  source: /note=“Description of Artificial Sequence: Synthetic polypeptide”
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSS

SEQ ID NO: 707 - DNA, 375 bp, Artificial Sequence
  source: /note=“Description of Artificial Sequence: Synthetic polynucleotide”
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagc

SEQ ID NO: 708 - DNA, 375 bp, Artificial Sequence
  source: /note=“Description of Artificial Sequence: Synthetic polynucleotide”
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 709 - protein, 451 aa, Artificial Sequence
  source: /note=“Description of Artificial Sequence: Synthetic polypeptide”
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 710 - protein, 11 aa, Artificial Sequence
  source: /note=“Description of Artificial Sequence: Synthetic peptide”
SSSQDISNYLN

SEQ ID NO: 711 - protein, 7 aa, Artificial Sequence
  source: /note=“Description of Artificial Sequence: Synthetic peptide”
YTSTLHL

SEQ ID NO: 712 - protein, 9 aa, Artificial Sequence
  source: /note=“Description of Artificial Sequence: Synthetic peptide”
QQYYNLPWT

SEQ ID NO: 713 - protein, 7 aa, Artificial Sequence
  source: /note=“Description of Artificial Sequence: Synthetic peptide”
SQDISNY

SEQ ID NO: 714 - protein, 3 aa, Artificial Sequence
  source: /note=“Description of Artificial Sequence: Synthetic peptide”
YTS

SEQ ID NO: 715 - protein, 6 aa, Artificial Sequence
  source: /note=“Description of Artificial Sequence: Synthetic peptide”
YYNLPW

SEQ ID NO: 716 - DNA, 1353 bp, Artificial Sequence
  source: /note=“Description of Artificial Sequence: Synthetic polynucleotide”
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagcgcgtccactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 717 - DNA, 1353 bp, Artificial Sequence
  source: /note=“Description of Artificial Sequence: Synthetic polynucleotide”
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 718 - protein, 107 aa, Artificial Sequence
  source: /note=“Description of Artificial Sequence: Synthetic polypeptide”
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIK

SEQ ID NO: 719 - DNA, 321 bp, Artificial Sequence
  source: /note=“Description of Artificial Sequence: Synthetic polynucleotide”
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaag

SEQ ID NO: 720 - DNA, 321 bp, Artificial Sequence
  source: /note=“Description of Artificial Sequence: Synthetic polynucleotide”
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaag

SEQ ID NO: 721 - protein, 214 aa, Artificial Sequence
  source: /note=“Description of Artificial Sequence: Synthetic polypeptide”
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 722 - DNA, 642 bp, Artificial Sequence
  source: /note=“Description of Artificial Sequence: Synthetic polynucleotide”
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 723 - DNA, 642 bp, Artificial Sequence
  source: /note=“Description of Artificial Sequence: Synthetic polynucleotide”
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 724 - protein, 125 aa, Artificial Sequence
  source: /note=“Description of Artificial Sequence: Synthetic polypeptide”
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSS

SEQ ID NO: 725 - DNA, 375 bp, Artificial Sequence
  source: /note=“Description of Artificial Sequence: Synthetic polynucleotide”
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagc

SEQ ID NO: 726 - DNA, 375 bp, Artificial Sequence
  source: /note=“Description of Artificial Sequence: Synthetic polynucleotide”
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 727 - protein, 451 aa, Artificial Sequence
  source: /note=“Description of Artificial Sequence: Synthetic polypeptide”
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 728 - DNA, 1353 bp, Artificial Sequence
  source: /note=“Description of Artificial Sequence: Synthetic polynucleotide”
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 729 - DNA, 1353 bp, Artificial Sequence
  source: /note=“Description of Artificial Sequence: Synthetic polynucleotide”
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 730 - protein, 107 aa, Artificial Sequence
  source: /note=“Description of Artificial Sequence: Synthetic polypeptide”
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIK

SEQ ID NO: 731 - DNA, 321 bp, Artificial Sequence
  source: /note=“Description of Artificial Sequence: Synthetic polynucleotide”
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaag

SEQ ID NO: 732 - DNA, 321 bp, Artificial Sequence
  source: /note=“Description of Artificial Sequence: Synthetic polynucleotide”
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaag

SEQ ID NO: 733 - protein, 214 aa, Artificial Sequence
  source: /note=“Description of Artificial Sequence: Synthetic polypeptide”
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 734 - DNA, 642 bp, Artificial Sequence
  source: /note=“Description of Artificial Sequence: Synthetic polynucleotide”
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 735 - DNA, 642 bp, Artificial Sequence
  source: /note=“Description of Artificial Sequence: Synthetic polynucleotide”
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 736 - DNA, 15 bp, Artificial Sequence
  source: /note=“Description of Artificial Sequence: Synthetic oligonucleotide”
aattacgggatgaac

SEQ ID NO: 737 - DNA, 15 bp, Artificial Sequence
  source: /note=“Description of Artificial Sequence: Synthetic oligonucleotide”
aactacggcatgaac

SEQ ID NO: 738 - DNA, 51 bp, Artificial Sequence
  source: /note=“Description of Artificial Sequence: Synthetic oligonucleotide”
tggattaacaccgacaccggggagcctacctacgcggacgatttcaaggga

SEQ ID NO: 739 - DNA, 51 bp, Artificial Sequence
  source: /note=“Description of Artificial Sequence: Synthetic oligonucleotide”
tggatcaacaccgacaccggcgagcctacctacgccgacgacttcaagggc

SEQ ID NO: 740 - DNA, 48 bp, Artificial Sequence
  source: /note=“Description of Artificial Sequence: Synthetic oligonucleotide”
aacccgccctactactacggaaccaacaacgccgaagccatggactac

SEQ ID NO: 741 - DNA, 48 bp, Artificial Sequence
  source: /note=“Description of Artificial Sequence: Synthetic oligonucleotide”
aaccccccttactactacggcaccaacaacgccgaggccatggactat

SEQ ID NO: 742 - DNA, 21 bp, Artificial Sequence
  source: /note=“Description of Artificial Sequence: Synthetic oligonucleotide”
ggattcaccctcaccaattac

SEQ ID NO: 743 - DNA, 21 bp, Artificial Sequence
  source: /note=“Description of Artificial Sequence: Synthetic oligonucleotide”
ggcttcaccctgaccaactac

SEQ ID NO: 744 - DNA, 18 bp, Artificial Sequence
  source: /note=“Description of Artificial Sequence: Synthetic oligonucleotide”
aacaccgacaccggggag

SEQ ID NO: 745 - DNA, 18 bp, Artificial Sequence
  source: /note=“Description of Artificial Sequence: Synthetic oligonucleotide”
aacaccgacaccggcgag

SEQ ID NO: 746 - DNA, 33 bp, Artificial Sequence
  source: /note=“Description of Artificial Sequence: Synthetic oligonucleotide”
agctctagtcaggatatctctaactacctgaac

SEQ ID NO: 747 - DNA, 33 bp, Artificial Sequence
  source: /note=“Description of Artificial Sequence: Synthetic oligonucleotide”
tcctccagccaggacatctccaactacctgaac

SEQ ID NO: 748 - DNA, 21 bp, Artificial Sequence
  source: /note=“Description of Artificial Sequence: Synthetic oligonucleotide”
tacactagcaccctgcacctg

SEQ ID NO: 749 - DNA, 21 bp, Artificial Sequence
  source: /note=“Description of Artificial Sequence: Synthetic oligonucleotide”
tacacctccaccctgcacctg

SEQ ID NO: 750 - DNA, 27 bp, Artificial Sequence
  source: /note=“Description of Artificial Sequence: Synthetic oligonucleotide”
cagcagtactataacctgccctggacc

SEQ ID NO: 751 - DNA, 27 bp, Artificial Sequence
  source: /note=“Description of Artificial Sequence: Synthetic oligonucleotide”
cagcagtactacaacctgccctggacc

SEQ ID NO: 752 - DNA, 21 bp, Artificial Sequence
  source: /note=“Description of Artificial Sequence: Synthetic oligonucleotide”
agtcaggatatctctaactac

SEQ ID NO: 753 - DNA, 21 bp, Artificial Sequence
  source: /note=“Description of Artificial Sequence: Synthetic oligonucleotide”
agccaggacatctccaactac

SEQ ID NO: 754 - DNA, 9 bp, Artificial Sequence
  source: /note=“Description of Artificial Sequence: Synthetic oligonucleotide”
tacactagc

SEQ ID NO: 755 - DNA, 9 bp, Artificial Sequence
  source: /note=“Description of Artificial Sequence: Synthetic oligonucleotide”
tacacctcc

SEQ ID NO: 756 - DNA, 18 bp, Artificial Sequence
  source: /note=“Description of Artificial Sequence: Synthetic oligonucleotide”
tactataacctgccctgg

SEQ ID NO: 757 - DNA, 18 bp, Artificial Sequence
  source: /note=“Description of Artificial Sequence: Synthetic oligonucleotide”
tactacaacctgccctgg

SEQ ID NO: 758 - DNA, 15 bp, Artificial Sequence
  source: /note=“Description of Artificial Sequence: Synthetic oligonucleotide”
aactacgggatgaac

SEQ ID NO: 759 - DNA, 51 bp, Artificial Sequence
  source: /note=“Description of Artificial Sequence: Synthetic oligonucleotide”
tggattaacaccgacaccggcgagcctacctacgccgacgactttaagggc

SEQ ID NO: 760 - DNA, 48 bp, Artificial Sequence
  source: /note=“Description of Artificial Sequence: Synthetic oligonucleotide”
aaccccccctactactacggcactaacaacgccgaggctatggactac

SEQ ID NO: 761 - DNA, 21 bp, Artificial Sequence
  source: /note=“Description of Artificial Sequence: Synthetic oligonucleotide”
ggcttcaccctgactaactac

SEQ ID NO: 762 - protein, 447 aa, Artificial Sequence
  source: /note=“Description of Artificial Sequence: Synthetic polypeptide”
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 763 - protein, 214 aa, Artificial Sequence
  source: /note=“Description of Artificial Sequence: Synthetic polypeptide”
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 764 - protein, 446 aa, Artificial Sequence
  source: /note=“Description of Artificial Sequence: Synthetic polypeptide”
QVQLKESGPGLVAPSQSLSITCTVSGFSLTAYGVNWVRQPPGKGLEWLGMIWDDGSTDYNSALKSRLSISKDNSKSQVFLKMNSLQTDDTARYYCAREGDVAFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 765 - protein, 220 aa, Artificial Sequence
  source: /note=“Description of Artificial Sequence: Synthetic polypeptide”
DIVMTQSPSSLAVSVGQKVTMSCKSSQSLLNGSNQKNYLAWYQQKPGQSPKLLVYFASTRDSGVPDRFIGSGSGTDFTLTISSVQAEDLADYFCLQHFGTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 766 - protein, 10 aa, Artificial Sequence
  source: /note=“Description of Artificial Sequence: Synthetic peptide”
GFTLTNYGMN

SEQ ID NO: 767-800 - skipped (34 entries)

SEQ ID NO: 801 - protein, 5 aa, Artificial Sequence
  source: /note=“Description of Artificial Sequence: Synthetic peptide”
SYNMH

SEQ ID NO: 802 - protein, 17 aa, Artificial Sequence
  source: /note=“Description of Artificial Sequence: Synthetic peptide”
DIYPGNGDTSYNQKFKG

SEQ ID NO: 803 - protein, 9 aa, Artificial Sequence
  source: /note=“Description of Artificial Sequence: Synthetic peptide”
VGGAFPMDY

SEQ ID NO: 804 - protein, 7 aa, Artificial Sequence
  source: /note=“Description of Artificial Sequence: Synthetic peptide”
GYTFTSY

SEQ ID NO: 805 - protein, 6 aa, Artificial Sequence
  source: /note=“Description of Artificial Sequence: Synthetic peptide”
YPGNGD

SEQ ID NO: 806 - protein, 118 aa, Artificial Sequence
  source: /note=“Description of Artificial Sequence: Synthetic polypeptide”
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSS

SEQ ID NO: 807 - DNA, 354 bp, Artificial Sequence
  source: /note=“Description of Artificial Sequence: Synthetic polynucleotide”
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagc

SEQ ID NO: 808 - protein, 444 aa, Artificial Sequence
  source: /note=“Description of Artificial Sequence: Synthetic polypeptide”
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 809 - DNA, 1332 bp, Artificial Sequence
  source: /note=“Description of Artificial Sequence: Synthetic polynucleotide”
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 810 - protein, 15 aa, Artificial Sequence
  source: /note=“Description of Artificial Sequence: Synthetic peptide”
RASESVEYYGTSLMQ

SEQ ID NO: 811 - protein, 7 aa, Artificial Sequence
  source: /note=“Description of Artificial Sequence: Synthetic peptide”
AASNVES

SEQ ID NO: 812 - protein, 9 aa, Artificial Sequence
  source: /note=“Description of Artificial Sequence: Synthetic peptide”
QQSRKDPST

SEQ ID NO: 813 - protein, 11 aa, Artificial Sequence
  source: /note=“Description of Artificial Sequence: Synthetic peptide”
SESVEYYGTSL

SEQ ID NO: 814 - protein, 3 aa, Artificial Sequence
  source: /note=“Description of Artificial Sequence: Synthetic peptide”
AAS

SEQ ID NO: 815 - protein, 6 aa, Artificial Sequence
  source: /note=“Description of Artificial Sequence: Synthetic peptide”
SRKDPS

SEQ ID NO: 816 - protein, 111 aa, Artificial Sequence
  source: /note=“Description of Artificial Sequence: Synthetic polypeptide”
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIK

SEQ ID NO: 817 - DNA, 333 bp, Artificial Sequence
  source: /note=“Description of Artificial Sequence: Synthetic polynucleotide”
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaag

SEQ ID NO: 818 - protein, 218 aa, Artificial Sequence
  source: /note=“Description of Artificial Sequence: Synthetic polypeptide”
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 819 - DNA, 654 bp, Artificial Sequence
  source: /note=“Description of Artificial Sequence: Synthetic polynucleotide”
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 820 - protein, 17 aa, Artificial Sequence
  source: /note=“Description of Artificial Sequence: Synthetic peptide”
DIYPGQGDTSYNQKFKG

SEQ ID NO: 821 - protein, 6 aa, Artificial Sequence
  source: /note=“Description of Artificial Sequence: Synthetic peptide”
YPGQGD

SEQ ID NO: 822 - protein, 118 aa, Artificial Sequence
  source: /note=“Description of Artificial Sequence: Synthetic polypeptide”
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSS

SEQ ID NO: 823 - DNA, 354 bp, Artificial Sequence
  source: /note=“Description of Artificial Sequence: Synthetic polynucleotide”
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagc

SEQ ID NO: 824 - protein, 444 aa, Artificial Sequence
  source: /note=“Description of Artificial Sequence: Synthetic polypeptide”
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 825 - DNA, 1332 bp, Artificial Sequence
  source: /note=“Description of Artificial Sequence: Synthetic polynucleotide”
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 826 - protein, 111 aa, Artificial Sequence
  source: /note=“Description of Artificial Sequence: Synthetic polypeptide”
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIK

SEQ ID NO: 827 - DNA, 333 bp, Artificial Sequence
  source: /note=“Description of Artificial Sequence: Synthetic polynucleotide”
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaag

SEQ ID NO: 828 - protein, 218 aa, Artificial Sequence
  source: /note=“Description of Artificial Sequence: Synthetic polypeptide”
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 829 - DNA, 654 bp, Artificial Sequence
  source: /note=“Description of Artificial Sequence: Synthetic polynucleotide”
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 830 - protein, 114 aa, Artificial Sequence
  source: /note=“Description of Artificial Sequence: Synthetic polypeptide”
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSA

SEQ ID NO: 831 - protein, 108 aa, Artificial Sequence
  source: /note=“Description of Artificial Sequence: Synthetic polypeptide”
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKR

SEQ ID NO: 832 - protein, 120 aa, Artificial Sequence
  source: /note=“Description of Artificial Sequence: Synthetic polypeptide”
EVQVLESGGGLVQPGGSLRLYCVASGFTFSGSYAMSWVRQAPGKGLEWVSAISGSGGSTYYADSVKGRFTISRDNSKNTLYLQMNSLRAEDTAVYYCAKKYYVGPADYWGQGTLVTVSSG

SEQ ID NO: 833 - protein, 113 aa, Artificial Sequence
  source: /note=“Description of Artificial Sequence: Synthetic polypeptide”
DIVMTQSPDSLAVSLGERATINCKSSQSVLYSSNNKNYLAWYQHKPGQPPKLLIYWASTRESGVPDRFSGSGSGTDFTLTISSLQAEDVAVYYCQQYYSSPLTFGGGTKIEVK

SEQ ID NO: 834-900 - skipped (67 entries)

SEQ ID NO: 901 - protein, 121 aa, Artificial Sequence
  source: /note=“Description of Artificial Sequence: Synthetic polypeptide”
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSS

SEQ ID NO: 902 - protein, 107 aa, Artificial Sequence
  source: /note=“Description of Artificial Sequence: Synthetic polypeptide”
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIK

SEQ ID NO: 903 - protein, 451 aa, Artificial Sequence
  source: /note=“Description of Artificial Sequence: Synthetic polypeptide”
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 904 - protein, 214 aa, Artificial Sequence
  source: /note=“Description of Artificial Sequence: Synthetic polypeptide”
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 905 - DNA, 363 bp, Artificial Sequence
  source: /note=“Description of Artificial Sequence: Synthetic polynucleotide”
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctcc

SEQ ID NO: 906 - DNA, 321 bp, Artificial Sequence
  source: /note=“Description of Artificial Sequence: Synthetic polynucleotide”
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaag

SEQ ID NO: 907 - DNA, 1353 bp, Artificial Sequence
  source: /note=“Description of Artificial Sequence: Synthetic polynucleotide”
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctccgctagcaccaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 908 - DNA, 642 bp, Artificial Sequence
  source: /note=“Description of Artificial Sequence: Synthetic polynucleotide”
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 909 - protein, 5 aa, Artificial Sequence
  source: /note=“Description of Artificial Sequence: Synthetic peptide”
SYGVD

SEQ ID NO: 910 - protein, 7 aa, Artificial Sequence
  source: /note=“Description of Artificial Sequence: Synthetic peptide”
GFSLSSY

SEQ ID NO: 911 - protein, 16 aa, Artificial Sequence
  source: /note=“Description of Artificial Sequence: Synthetic peptide”
VIWGGGGTYYASSLMG

SEQ ID NO: 912 - protein, 5 aa, Artificial Sequence
  source: /note=“Description of Artificial Sequence: Synthetic peptide”
WGGGG

SEQ ID NO: 913 - protein, 13 aa, Artificial Sequence
  source: /note=“Description of Artificial Sequence: Synthetic peptide”
HAYGHDGGFAMDY

SEQ ID NO: 914 - protein, 11 aa, Artificial Sequence
  source: /note=“Description of Artificial Sequence: Synthetic peptide”
RASESVSSNVA

SEQ ID NO: 915 - protein, 7 aa, Artificial Sequence
  source: /note=“Description of Artificial Sequence: Synthetic peptide”
SESVSSN

SEQ ID NO: 916 - protein, 7 aa, Artificial Sequence
  source: /note=“Description of Artificial Sequence: Synthetic peptide”
GASNRAT

SEQ ID NO: 917 - protein, 3 aa, Artificial Sequence
  source: /note=“Description of Artificial Sequence: Synthetic peptide”
GAS

SEQ ID NO: 918 - protein, 9 aa, Artificial Sequence
  source: /note=“Description of Artificial Sequence: Synthetic peptide”
GQSYSYPFT

SEQ ID NO: 919 - protein, 6 aa, Artificial Sequence
  source: /note=“Description of Artificial Sequence: Synthetic peptide”
SYSYPF

SEQ ID NO: 920 - protein, 124 aa, Artificial Sequence
  source: /note=“Description of Artificial Sequence: Synthetic polypeptide”
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSS

SEQ ID NO: 921 - protein, 107 aa, Artificial Sequence
  source: /note=“Description of Artificial Sequence: Synthetic polypeptide”
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 922 - protein, 6 aa, Artificial Sequence
  source: /note=“Description of Artificial Sequence: Synthetic 6xHis tag”
HHHHHH